(19) United States
(12) Patent Application Publication
(10) Pub. No.: US 2005/0009031 A1
(43) Pub. Date: Jan. 13, 2005

(54) Genes and polymorphisms on chromosome 10 associated with Alzheimer's disease and other neurodegenerative diseases

(76) Inventors: Kenneth David Becker, San Diego, CA (US); Gonul Velicelebi, San Diego, CA (US); Kathryn J. Elliott, San Diego, CA (US); Xin Wang, San Diego, CA (US); Rudolph E. Tanzi, Hull, MA (US); Lars Bertram, Boston, MA (US); Aleister J. Saunders, Philadelphia, PA (US); Kristina M. Mullin, Weymouth, MA (US); Andrew Joseph Sampson, Oakwood, OH (US)

Correspondence Address: FISH & RICHARDSON, PC, 12390 EL CAMINO REAL, SAN DIEGO, CA, 92130-2081, US

(21) Appl. No.: 10/600,009

(22) Filed: Jun. 18, 2003

Related U.S. Application Data
(63) Continuation-in-part of application No. 10/282,174, filed on Oct. 25, 2002.
(63) Continuation-in-part of application No. PCT/US02/34679, filed on Oct. 25, 2002.
(60) Provisional application No. 60/339,525, filed on Oct. 25, 2001.
(60) Provisional application No. 60/338,010, filed on Nov. 8, 2001.
(60) Provisional application No. 60/336,929, filed on Nov. 8, 2001.
(60) Provisional application No. 60/338,363, filed on Nov. 9, 2001.
(60) Provisional application No. 60/337,052, filed on Dec. 4, 2001.
(60) Provisional application No. 60/368,919, filed on Mar. 28, 2002.

(51) Int. Cl. C12Q 1/68; C07H 21/04

(52) U.S. Cl. 435/6; 536/24.3

(57) ABSTRACT

Isolated nucleic acid molecules containing polymorphisms in genes involved in neurodegenerative diseases are provided. Probes, primers, kits and methods for detection of polymorphisms in genes involved in neurodegenerative disease are provided. Methods based on detecting such polymorphisms for prognosticating, determining the occurrence, profiling drug response and drug discovery are also provided. Methods of screening for agents that modulate expression and/or activity of genes involved in neurodegenerative diseases, and of screening for agents that modulate a biological event characteristic of a neurodegenerative disease are further provided.

### RELATED APPLICATIONS

This application is a continuation-in-part of U.S. application Ser. NO. 10/282,174, filed Oct. 25, 2002, entitled “Genes and Polymorphisms on Chromosome 10 Associates with Alzheimer's Disease and Other Neurodegenerative Diseases,” and also a continuation-in-part of and International PCT Application US 02/34679 entitled “Genes and Polymorphisms on Chromosome 10 Associated with Alzheimer's Disease and Other Neurodegenerative Diseases,” filed Oct. 25, 2002.

U.S. application Ser. No. 10/282,174, claims benefit of priority under §119(e) to: U.S. Provisional Application Ser. No. 60/339,525, filed Oct. 25, 2001, entitled “Genes and Polymorphisms on Chromosome 10 Associates with Alzheimer's Disease and Other Neurodegenerative Diseases,” to U.S. Provisional Application Ser. No. 60/338,010, filed Nov. 8, 2001, entitled “Genes and Polymorphisms on Chromosome 10 Associated with Alzheimer's Disease and Other Neurodegenerative Diseases,” to U.S. Provisional Application Ser. No. 60/336,929, filed Nov. 8, 2001, entitled “Polymorphic Urokinase Plasminogen Activator Genes as Genetic Markers for Neurodegenerative Disease,” to U.S. Provisional Application Ser. No. 60/338,363, filed Nov. 9, 2001, entitled “Polymorphic Urokinase Plasminogen Activator Genes and Methods Using the Same,” to U.S. Provisional Application Ser. No. 60/337,052, filed Dec. 4, 2001, entitled “Polymorphic Urokinase Plasminogen Activator Genes and Methods Using the Same,” and to U.S. Provisional Application Ser. No. 60/368,919, filed Mar. 28, 2002, entitled “Genes and Polymorphisms on Chromosome 10 Associated with Alzheimer's Disease and Other Neurodegenerative Diseases.” The subject matter of each of these applications is incorporated herein by reference in its entirety.

The subject matter of each of the following applications also is incorporated herein by reference in its entirety: U.S. Provisional Application Ser. No. 60/348,065, filed Oct. 25, 2001, entitled “Genetic Markers for Alzheimer's Disease and Methods of Using the Same”, and U.S. Provisional Application Ser. No. 60/336,983, filed Nov. 2, 2001, entitled “Genetic Markers for Alzheimer's Disease and Methods of Using the Same,” U.S. Patent Application Ser. No. 10/281,456 entitled “Genetic Markers for Alzheimer's Disease and Methods Using the Same,” filed Oct. 25, 2002; and International PCT Application US 02/34679 entitled “Genes and Polymorphisms on Chromosome 10 Associated with Alzheimer's Disease and Other Neurodegenerative Diseases,” filed Oct. 25, 2002.

### RELATED APPLICATIONS

Subject matter of this application was conducted with support from the United States Government under Grant Nos. 1 RO1MH60009 (NIMH) and 5P5OAG05134 (NIA). Thus, the U.S. Government may retain certain rights in such subject matter.

### FIELD OF THE INVENTION

The field of the invention involves genes and polymorphisms that are associated with neurodegenerative diseases. Probes, primers and kits for detection of polymorphisms are provided. Methods based on detecting such polymorphisms for prognosticating, determining the risk for or occurrence of neurodegenerative disease, profiling drug response and drug discovery are also provided. The invention also relates to polymorphisms of the IDE, KNSL1, PLAU, SNCG, LIPA and TNFRSF6 genes and polymorphic proteins encoded by these genes.

### BACKGROUND OF THE INVENTION

Neurodegenerative diseases are genetically complex, heterogeneous disorders that have many different etiologies. Many are hereditary, some are secondary to toxic or metabolic processes, and others result from infections or have no known etiology. Neurodegenerative diseases are often age associated, chronic and progressive without known treatment 37481-3308B modalities. These diseases are characterized by abnormalities of relatively specific regions of the brain and populations of neurons. The affected cell groups in the different diseases determine the clinical phenotype of the illnesses. Examples of neurodegenerative diseases include Motor Neuron diseases such as Amyotrophic Lateral Sclerosis (ALS), Dementing Illnesses including Alzheimer's disease (AD), Parkinsonian syndromes such as Parkinson's disease (PD), Huntington's disease (HD), and Prion diseases.

Individuals with dementing illnesses usually present with gradual loss of memory followed by progressive deterioration of thought, judgement, language skills, visual-spatial perception, mood, and the ability to manage personal affairs. These patients become severely demented and typically die of intercurrent medical illnesses, such as pneumonia. There are many causes of dementia including primary cortical degenerative disorders (AD, Pick's disease, and Lewy body disorders), cerebrovascular disease (multi-infarct dementia), sub-cortical degenerative disorders (Multiple System Atrophy, Huntington's disease and Progressive Supranuclear Palsy), infections (Neurosyphilis, AIDS), prion disorders, toxic and metabolic disorders (alcohol, hypothyroidism), tumors, and brain injury.

Parkinsonian symptoms can be present in several neurodegenerative disorders. The classical Parkinsonian syndrome is Parkinson's disease (PD), which is characterized by slowness of voluntary movement (Bradykinesia), rigidity, and tremor. This disorder generally affects individuals over 60 year of age and affects males and females equally. Cognitive deficits (dementia) are present in only a minority of patients, perhaps up to 10%. Parkinsonian syndromes were also recognized in the early twentieth century following the influenza pandemic and are referred to as Post Encephalitic Parkinsonism. Many survivors of the viral infection developed parkinsonism months to years later and at an earlier age of onset than PD, although the neuropathological changes have some similarities to PD. Experimental parkinsonism has been created by inadvertent exposure to the mitochondrial toxin called MPTP, an analogue of meperidine which was inadvertently made as a designer drug of abuse.

The vast majority of PD patients are sporadic although several autosomal dominant pedigrees of parkinsonism have been identified. Recently, it was shown that a protein called α-synuclein is present in Lewy bodies and that a few families with familial PD have a point mutation in the gene on chromosome 4 which encodes α-synuclein.

The neuropathological changes of PD are loss of the pigmented neurons in the substantia nigra, loss of pigment in the remaining neurons, and the presence of intracytoplasmic inclusions called Lewy bodies. This selective neuronal degeneration results in abnormalities of dopamine in the striatum and loss of dopamine in the basal ganglia motor circuit. Lewy bodies are found in the substantia nigra (midbrain) and locus coeruleus (pons). Lewy bodies are eosinophilic spherical inclusions which often have a clear halo surrounding them, pushing aside the neuromelanin pigment. Ultrastructurally, Lewy bodies are filamentous structures.

While many PD patients with dementia have no morphological abnormalities to account for the dementia, there is a subset of patients with dementia and parkinsonism who have widespread Lewy bodies in the brainstem and the cortex. This disease has been referred to as Diffuse Lewy Body disease. It has been shown that many patients with dementia and the morphological findings of typical Alzheimer's disease also have Lewy bodies in specific neuronal populations.

In monkeys and human intravenous drug users, the systemic administration of MPTP (1-methyl-4-phenyl-1,2,3,6-tetrahydropyridine) produces parkinsonian features. MPTP is converted to the toxic metabolite MPP+ which is taken up by dopamine systems and apparently inhibits the mitochondrial complex I leading to ATP depletion and generation of oxygen free radicals. This has led to theories that oxidative stress (i.e., the generation of free radicals) may play a role in PD. There have also been hypotheses suggesting age, other toxic processes, and other genetic factors to explain the pathogenesis of PD.

Huntington's disease (HD) is an autosomal dominant neurodegenerative disorder which is characterized by involuntary movements (chorea), dementia, and behavioral abnormalities. HD usually occurs in individuals over 40 years of age with abnormal movements and behavioral changes and over time the chorea progresses and rigidity and abnormal eye movements develop. In the end stage, nearly all HD patients develop cognitive impairments (dementia), personality changes, and a variety of psychological symptoms including irritability and depression, and eventually become mute.

Initially, genetic linkage was established between DNA markers and the distal region of the short arm of chromosome 4. This linkage was further defined by the demonstration of the HD gene IT15. The protein that is encoded by this gene is called huntingtin which contains a CAG triplet repeat which is expanded in the disorder. HD patients have greater than 37 CAG triplet repeats whereas normal alleles have less than 37. The extent of expansion is correlated with both age of onset and severity of disease such that juvenile onset HD patient have extremely large CAG expansions (up to 100) and very severe disease. Huntingtin is expressed in nearly all cells of the body, yet HD is primarily a disorder of a specific subset of neurons in the brain.

HD shows selective neuronal vulnerability. Medium spiny neurons in the striatum are lost whereas medium aspiny neurons in the same location are spared. The gross features of HD include marked atrophy of the striatum (caudate and putamen) and generalized cortical atrophy with decreased brain weight. The medium spiny neurons are principally GABAergic. In the neocortex, there is loss of neurons in layers III, V and VI. Recently, it has been shown that neurons in HD patients, both in affected neuronal populations and those not currently understood to be involved in the disease, have intranuclear inclusions which consist of cleaved fragments of mutant huntingtin with the expanded triplet repeat. The mechanism of this intranuclear aggregation of mutant huntingtin is unknown although it appears to be a universal feature in the triplet repeat disorders which are a topic of intense research.

A group of invariably fatal neurodegenerative diseases are caused by pathogenic agents termed prions. Prion diseases take the form of genetic, infectious, or sporadic disorders, all of which are believed to involve mutations in the genes which encode the prion protein and/or prion-like proteins. Prion diseases include the animal diseases such as bovine spongiform encephalopathy (BSE), scrapie of sheep, transmissible mink encephalopathy (TME), chronic wasting disease (CWD), and feline spongiform encephalopathy (FSE). There are also a number on known human prion diseases including iatrogenic (i), variant (v), familial (f), and sporadic (s) Creutzfeldt-Jakob disease (CJD); Kuru; Gerstmann-Straussler-Scheinker disease (GSS); and fatal familial insomnia (FFI).

Prion diseases are characterized by rapidly progressive dementia sometimes combined with cerebellar ataxia. Morphological changes associated with prion diseases include spongiform degeneration and astrocytic gliosis. Most cases of CJD and a few cases of GSS can be classified as sporadic. In these patients, mutations of the PrP gene are not found. How prions causing disease arise in patients with sporadic forms is unknown, however, hypotheses include horizontal transmission of prions from humans or animals, somatic mutation of the PrP gene, and spontaneous conversion of PrP^{c} into PrP^{Sc}. More than 20 mutations of the PrP gene (Prnp) are now known to cause inherited human prion diseases, and significant genetic linkage has been established for five of these mutations. The P102L mutation was the first PrP mutation to be genetically linked to GSS and is found in many GSS families throughout the world.

The particular dementing illness of Alzheimer's disease (AD) is a devastating neurodegenerative progressive disorder, which is the 37481-3308B predominant cause of dementia in people over 65 years of age. Clinical symptoms of the disease typically begin with subtle short term memory problems. As the disease progresses, the difficulty with memory, language and orientation worsen to the point of interfering with the ability of the person to function independently. Other symptoms, which are variable, include myoclonus and seizures. Duration of AD from the first symptoms of memory loss until death is 10 years on average. AD always results in death, often from respiratory-related illness.

The pathology in AD is confined exclusively to the central nervous system (CNS). The AD brain is characterized by the presence of amyloid deposits and neurofibrillary tangles (NFT). Amyloid deposits are also found associated with the vascular system of the CNS and as focal deposits in the parenchyma. The major molecular component of an amyloid deposit is a highly hydrophobic peptide called A-beta peptide. For example, in Alzheimer's disease the β-amyloid precursor protein (APP), a Cu2+ binding protein, undergoes cleavage during oxidative stress. Cleavage of APP can result in the formation of the A-beta peptide fragment which is thought to be responsible for the formation of senile plaques, a pathological hallmark of Alzheimer's disease. This peptide aggregates into filaments in an anti-beta-pleated structure. Aggregated A-beta may be the primary agent responsible for disease progression, as the accumulation of aggregates is toxic to the brain (Small et al. (1 999) J. Neurochem. 73:443-449). Although A-beta is the major component of AD amyloid, other proteins have also been found associated with the amyloid, e.g., alpha-1-anti-chymotrypsin (Abraham et al. (1988) Cell 52:487-501), cathepsin D (Cataldo et al. (1990) Brain Res. 513:181-192), non-amyloid component protein (Ueda et al. (1 993) Proc. Natl. Acad. Sci. U.S.A. 90:11282-11286), apolipoprotein E (apoE) (Namba et al. (1991) Brain Res. 541:163-166; Wisniewski & Frangione (1992) Neurosci. Lett. 135:235-238; Strittmatter et al. (1993) Proc. Natl. Acad. Sci. U.S.A. 90:1977-1 98 1), apolipoprotein J (Choi-Mura et al. (1 992) Acta Neuropathol. 83:260-264; McGeer et al. (1992) Brain Res. 579:337-341), heat shock protein 70 (Hamos et al. (1 991) Neurology 41:345-350), complement components (McGeer & Rogers (1992) Neurology 43:447-449), alpha2-macroglobin (Strauss et al. (1992) Lab. Invest. 66:223-230), interleukin-6 (Strauss et al. (1992) Lab. Invest. 66:223-230), proteoglycans (Snow et al. (1987) Lab. Invest. 58:454-458), and serum amyloid P (Coria et al. (1988) Lab. Invest. 58:454-458).

Plaques are often surrounded by astrocytes and activated microglial cells expressing immune-related proteins, such as the MHC class II glycoproteins HLA-DR, HLA-DP, and HLA-DQ, as well as MHC class I glycoproteins, interleukin-2 (IL-2) receptors, and IL-1. Also surrounding many plaques are dystrophic neurites, which are nerve endings containing abnormal filamentous structures.

The characteristic Alzheimer's NFTs consist of abnormal filaments bundled together in neuronal cell bodies. “Ghost” NFTs are also observed in AD brains, which presumably mark the location of dead neurons. Other neuropathological features include granulovascular changes, neuronal loss, gliosis and the variable presence of Lewy bodies.

The destructive process of the disease is evident on a gross level in the AD brain to the extent that in late-stage AD, ventricular enlargement and shrinkage of the brain can be observed by magnetic resonance imaging. The cells remaining at autopsy are grossly different from those of a normal brain and the brain is characterized by extensive gliosis and neuronal loss. Neurons which were possibly involved in initiating events, are absent; and other cell types, such as the activated microglial cells and astrocytes, have gene expression patterns not observed in the normal brain. Thus, the amyloid plaque structures and NFTs observed at autopsy are most likely the end-products of a lengthy disease process, far removed from the initiating events of AD.

Accordingly, attempts to use biochemical methods to identify key proteins and genes in the initiating steps of the disease are hampered by the fact that it is not possible to actually observe these critical initiating events. Rather, biochemical dissection of the AD brain at autopsy is akin to molecular archeology, attempting to reconstruct the pathogenic pathway by comparing the normal brain to the end-stage disease brain.

Determining the genetic basis of neurodegenerative diseases, such as AD, is also made difficult as these are genetically complex and heterogeneous disorders. Also, because AD is relatively common in the elderly, clustering of cases in a family may occur by chance, representing possible confounding non-allelic genetic heterogeneity, or etiologic heterogeneity with genetic and non-genetic cases co-existing in the same kindred. In addition, the diagnosis of AD is also confounded with other dementing diseases and conditions common in the elderly, including dementia-causing conditions such as strokes, microvascular disease, brain tumors, thyroid dysfunction, drug reactions, severe depression and a host of other conditions that can cause intellectual deficits in the elderly. Furthermore, many of the pathological features of AD are not unique to the disease and also occur in the brains of normal aged individuals and are associated with diseases such as Guam Parkinson disease, dementia pugilistica and progressive supranuclear palsy. For example, the twisted filaments that form NFTs also occur in certain tangles associated with other diseases such as Pick's disease.

Despite these problems, it has been found that roughly 40% of early-onset AD (less than about 65 years) is attributable to missense mutations in three genes (APP, PSEN1 and PSEN2). However, early-onset cases only account for approximately 1-2% of all AD cases. The genetic basis of late onset AD has proven more difficult to disentangle (D. Blacker, R. E. Tanzi (1998) Arch Neurol 55:294). Substantial evidence has suggested that inherited genetic defects are involved in late-onset AD. Families with multiple late-onset AD cases have been described (Bird et al. (1989) Ann. Neurol 25:12-25; Heston & White (1978) Behavior Genet. 8:315-331; Pericak-Vance et al. (1988) Exp. Neurol. 102:271-279).

To date, only one genetic risk factor, a common polymorphism in the apolipoprotein E (APOE) gene, has been replicated in independent samples in late-onset AD (L. A. Farrer et al. (1997) JAMA 278:1349; D. Blacker et al. (1 997) Neurology 48:139). However, approximately half of AD cases do not have the APOE epsilon 4 allele found in several other families with high incidence of AD, including the Volga German (VG) kindreds (Brousseau et al. (1 994) Neurology 342; Kuusisto et al. (1994) Brit. Med. J. 309:363; Tsai et al. (1994) Am. J. Hum. Genet. 54:643; Liddel et al. (1 994) J. Med. Genet. 31:197; Cook et al. (1979) Neurology 29:1402-1412; Bird et al.(1 988) Ann. Neurol. 23:25-31; Bird et al. (1989) Ann. Neurol. supra). The known AD loci have been excluded as possible causes of the discrepancy (Schellenberg et al. (1992) Science 258:668; Lannfelt et al. (1993) Nat. Genet. 4:218-219; Van Duijn et al. (1994) Am. J Hum. Genet. 55:714-727; Schellenberg et al. (1988) Science 241:1507; Schellenberg et al. (1 991) Am. J. Hum. Genet. 48:563; Schellenberg et al. (1991) Am. J. Hum. Genet. 49:511-517 (1991); Kamino et al. (1992) Am. J. Hum. Genet. 51:998; Schellenberg et al. (1 993) Am J. Hum. Genet. 53:619; Schellenberg et al. (1 992) Ann. Neurol. 31:223; Yu et al. (1994) Am. Hum. Genet. 54:631). Also, there is evidence that genetic factors other than APOE contribute to the risk for late onset AD. A study modeling AD as a quantitative trait estimated at least four additional genetic susceptibility loci for the disease (E. Daw et al. (2000) Am J Hum Genet 66:196).

### SUMMARY OF THE INVENTION

An understanding of the genes that are responsible for AD and other neurodegenerative disorders, along with useful genetic markers and mutations in these genes, will allow for methods of detecting an altered level of risk and/or determining the occurrence of AD and other neurodegenerative diseases and the development of therapeutics that target these alterations. Therefore, provided herein are methods for using polymorphic markers to detect a predisposition to, or protection against, the manifestation of or the occurrence of neurodegenerative disease, such as Alzheimer's disease, and the like. The ultimate goals are the elucidation of pathological pathways, developing new diagnostic assays, determining genetic profiles for positive responses to therapeutic drugs, identifying new potential drug targets and identifying new drug candidates. Based on proximity to linkage peaks on chromosome 10 as determined by genetic mapping of DNA samples from Alzheimer's disease patients and their families, positional candidate genes for neurodegenerative disease, including Alzheimer's disease, have been identified. In a particular embodiment, the methods provided herein are useful in diagnosing late-onset Alzheimer's disease (LOAD). These candidate genes include uPA (Urokinase plasminogen activator; also referred to as PLAU), SNCG (human γ-synuclein), IDE (insulin-degrading enzyme), KNSL1 (human Kinesin-like protein 1), TNFRSF6 (Tumor Necrosis Factor Receptor-SF6) and LIPA (lysosomal acid lipase). High throughput DNA sequencing identified polymorphic regions, including single nucleotide polymorphisms, in these genes and surrounding regions in chromosome 10.

In one embodiment, provided herein are isolated nucleic acid molecules, comprising at least 14, 16, 18, 20, 22, 24, 26, 28, 30, 35, 40, 45, 50, 60, 70, 80, 90 or 100 contiguous nucleotides of a KNSL1 gene allele corresponding to a sequence of at least 14 contiguous nucleotides that includes nucleotide position 41014 and/or 41015 of SEQ ID NO:347, or the complement thereof, wherein the sequence of at least 14 contiguous nucleotides comprises one or more nucleotides inserted between positions 41014 and 41015, or the complementary positions thereof. In one embodiment, the at least 14, 16, 18, 20, 22, 24, 26, 28, 30, 35, 40, 45, 50, 60, 70, 80, 90 or 100 contiguous nucleotides comprise the nucleotide sequence AATTT, or the complement thereof, inserted between positions 41014 and 41015, or the complementary positions thereof. In another embodiment, the 14, 1 6, 1 8, 20, 22, 24, 26, 28, 30, 35, 40, 45, 50, 60, 70, 80, 90 or 100 contiguous nucleotides comprise a sequence of 5, 6, 7, 8 or 9 contiguous nucleotides of SEQ ID NO: 347, or the complement thereof, within the sequence of nucleotides from position 41011 to 41018, except that between the nucleotides at positions 41014 and 41015 the sequence AATTT is inserted, or the complement thereof is inserted in the complementary sequence. Also provided is an isolated nucleic acid molecule, wherein the sequence of at least 14 contiguous nucleotides comprises a sequence of at least 14 contiguous nucleotides of SEQ ID NO:347, or the complement thereof, that comprises said nucleotide position 41014 and/or 41015 and the nucleotide sequence AATTT inserted between nucleotide positions 41014 and 41015. For each of these isolated nucleic acids, an other nucleic acids provided herein, can further comprise a coding nucleotide sequence operatively linked to a promoter. Also provided are vectors comprising the nucleic acid molecules provided herein, including the nucleic acids describe above. Also provided are cells comprising any one or more of the nucleic acid molecules provided herein, wherein the nucleic acid molecule is heterologous to the cell. Also provided herein are non-human transgenic animals, comprising any of the nucleic acid molecules provided herein, wherein the nucleic acid molecule is a transgenic element of the animal.

Also provided are isolated nucleic acid molecules, comprising a coding nucleotide sequence operatively linked to a promoter, wherein the coding nucleotide sequence is contained within a nucleotide sequence comprising a portion of a KNSL1 gene allele that comprises a sequence of at least 14, 16, 18, 20, 22, 24, 26, 28, 30, 35, 40, 45, 50, 60, 70, 80, 90 or 1 00 contiguous nucleotides of a KNSL1 gene allele but does not contain a contiguous sequence of a complete KNSL1 gene allele,
wherein the sequence of at least 14 contiguous nucleotides corresponds to a sequence of 14 contiguous nucleotides that includes nucleotide position 41014 and/or 41015 of SEQ ID NO:347, or the complement thereof, wherein the sequence does or does not contain one or more nucleotides inserted between nucleotide positions 41014 and 41015, or the complementary positions thereof. In one embodiment, the sequence of at least 14, 16, 18, 20, 22, 24, 26, 28, 30, 35, 40, 45, 50, 60, 70, 80, 90 or 100 contiguous nucleotides that includes nucleotide position 41014 and/or 41015 of SEQ ID NO:347, or the complement thereof, does or does not contain the nucleotide sequence AATTT, or the complement thereof, inserted between nucleotide positions 41014 and 41015, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:347, or the complement thereof, within the sequence of nucleotides from position 41010 to 41019, wherein the sequence does or does not contain one or more nucleotides inserted between nucleotide positions 41014 and 41015, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:347, or the complement thereof, wherein the sequence does or does not contain one or more nucleotides inserted between nucleotide positions 41014 and 41015, or the complementary positions thereof. For each of these nucleic acid molecules, the coding nucleotide sequence can encode a reporter molecule that is not a KNSL1 protein or the coding nucleotide sequence encodes a KNSL1 protein. For each of these nucleic acid molecules, the promoter can comprise a promoter that is heterologous to a KNSL1 gene, or can comprise a KNSL1 gene promoter.

In another embodiment, provided herein are isolated nucleic acid molecules comprising at least 50, 60, 70, 80, 90 or 100 contiguous nucleotides of a KNSL1 gene allele corresponding to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof. In one embodiment, the sequence of at least 50, 60, 70, 80, 90 or 100 contiguous nucleotides comprises a 6-, 7- or 8-bp poly-T sequence, or the complement thereof, inserted between nucleotides at positions 1 33354 and 133355, or the complementary positions thereof. In another embodiment, the 50, 60, 70, 80, 90 or 100 contiguous nucleotides comprise a sequence of 5, 6, 7, 8 or 9 contiguous nucleotides of SEQ ID NO: 484,.or the complement thereof, within the sequence of nucleotides from position 133351 to 133358, except that between the nucleotides at positions 1 33354 and 133355 a 6-, 7-, or 8-bp polyT sequence is inserted, or the complement thereof is inserted in the complementary sequence. Also provided is an isolated nucleic acid molecule, wherein the sequence of at least 50, 60, 70, 80, 90 or 100 contiguous nucleotides comprises a sequence of at least 50 contiguous nucleotides of SEQ ID NO:484, or the complement thereof, that comprises said nucleotide position 1 33354 and/or 133355 and a 6-, 7-, or 8-bp polyT nucleotide sequence inserted between nucleotide positions 133354 and 133355. In a particular embodiment for each of these isolated nucleic acid molecules, a 7-bp polyT nucleotide sequence, or the complement thereof, is inserted between nucleotide positions 133354 and 133355, or the complementary positions thereof. In other embodiments, each of these nucleic acid. molecules can further comprise a coding nucleotide sequence operatively linked to a promoter.

Also provided herein are isolated nucleic acid molecules, comprising a coding nucleotide sequence operatively linked to a promoter, wherein the coding nucleotide sequence is contained within a nucleotide sequence comprising a portion of a KNSL1 gene allele that comprises a sequence of at least 50, 60, 70, 80, 90 or 100 contiguous nucleotides of a KNSL1 gene allele but does not contain a contiguous sequence of a complete KNSL1 gene allele, wherein the sequence of at least 50 contiguous nucleotides corresponds to a sequence of 50 contiguous nucleotides that includes nucleotide position 133354 and/or 133355 of SEQ ID NO:484, or the complement thereof, wherein the sequence does or does not contain one or more nucleotides inserted between nucleotide positions 133354 and 133355; and wherein the isolated nucleic acid includes sequence that is heterologous to the KNSL1 gene allele. In one embodiment, the sequence of 50, 60, 70, 80, 90 or 100 contiguous nucleotides that includes nucleotide position 133354 and/or 133355 of SEQ ID NO:484, or the complement thereof, does or does not contain a 6-, 7- or 8-bp polyT sequence inserted between nucleotide positions 133354 and 133355. In another embodiment, the sequence of at least 50, 60, 70, 80, 90 or 100 contiguous nucleotides comprises a sequence of 5, 6, 7, 8 or 9 contiguous nucleotides of SEQ ID NO:484, or the. complement thereof, within the sequence of nucleotides from position 1 33350 to 133359, wherein the sequence does or does not contain one or more nucleotides inserted between nucleotide positions 133354 and 133355. In yet another embodiment, the sequence of at least 50, 60, 70, 80, 90 or 1 00 contiguous nucleotides comprises a sequence of at least 50 contiguous nucleotides of SEQ ID NO:484, or the complement thereof, that comprises said nucleotide at position 1 33354 and/or 133355 and does or does not one or more nucleotides inserted between nucleotide positions 1 33354 and 1 33355. For each of these nucleic acid molecules, the coding nucleotide sequence can encode a reporter molecule that is not a KNSL1 protein, or the coding nucleotide sequence can encode a KNSL1 protein. For each of these embodiments, the promoter can comprise a promoter that is heterologous to a KNSL1 gene, or the promoter can comprise a KNSL1 gene promoter. Also provided are vectors comprising the nucleic acid molecules provided herein, including the nucleic acids describe above. Also provided are cells comprising any one or more of the nucleic acid molecules provided herein, wherein the nucleic acid molecule is heterologous to the cell. Also provided herein are non-human transgenic animals, comprising any one of the nucleic acid molecules provided herein, wherein the nucleic acid molecule is a transgenic element of the animal. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO;484 for each of the methods and applications provided herein that assess the polymorphism at position 133354/133355 of SEQ ID NO:484.

Also provided herein, are isolated nucleic acid molecules, comprising at least 50, 60, 70, 80, 90 or 100 contiguous nucleotides of a KNSL1 gene allele corresponding to a sequence of at least 50 contiguous nucleotides that includes nucleotide position 133354 of SEQ ID NO:484, or the complement thereof, except that the nucleotide at position 133354, or the complementary position thereof, is deleted. In one embodiment, the 50, 60, 70, 80, 90 or 100 contiguous nucleotides comprise a sequence of 5, 6, 7, 8 or 9 contiguous nucleotides of SEQ ID NO:484, or the complement thereof, within the sequence of nucleotides from position 133351 to 133357, except that the nucleotide at position 133354, or the complementary position thereof, is deleted. In another embodiment, the sequence of at least 50, 60, 70, 80, 90 or 100 contiguous nucleotides comprises a sequence of at least 50 contiguous nucleotides of SEQ ID NO: 484, or the complement thereof, except that the nucleotide at position 1 33354, or the complementary position thereof, is deleted.

In another embodiment, provided herein are isolated nucleic acid molecules, comprising at least 14, 16, 18, 20, 22, 24, 26, 28, 30, 35, 40, 45, 50, 60, 70, 80, 90 or 100 contiguous nucleotides of an IDE gene allele corresponding to a sequence of at least 14 contiguous nucleotides that includes nucleotide position 122260 of SEQ ID NO:484, or the complement thereof, except that the nucleotide at position 1 22260 is replaced with a G, T or C, or is replaced with a C, A or G in a complementary sequence thereof. In one embodiment, the 14, 16, 18, 20, 22, 24, 26, 28, 30, 35, 40, 45, 50, 60, 70, 80, 90 or 100 contiguous nucleotides comprise a sequence of 5, 6, 7, 8 or 9 contiguous nucleotides of SEQ ID NO:484, or the complement thereof, within the sequence of nucleotides from position 122256 to 1 22264, except that the nucleotide at position 122260 is replaced with a G, T or C, or is replaced with a C, A or G in a complementary sequence thereof. In another embodiment, the sequence of at least 14, 16, 1 8, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in a complementary sequence thereof. In a particular embodiment, the nucleotide at position 122260 is replaced with a G, or is replaced with a C in a complementary position thereof. These neucleic acid molecules, can further comprise a coding nucleotide sequence operatively linked to a promoter.

Also provided herein are isolated nucleic acid molecules, comprising a coding nucleotide sequence operatively linked to a promoter, wherein the coding nucleotide sequence is contained within a nucleotide sequence comprising a portion of an IDE gene allele that comprises a sequence of at least 14, 16, 18, 20, 22, 24, 26, 28, 30, 35, 40, 45, 50, 60, 70, 80, 90 or 100 contiguous nucleotides of an IDE gene allele but does not contain a contiguous sequence of a complete IDE gene allele, wherein the sequence of at least 14 contiguous nucleotides corresponds to a sequence of 14 contiguous nucleotides that includes nucleotide position 122260 of SEQ ID NO:484, or the complement thereof, wherein the nucleotide at position 1 22260 is A, T, C or G; and wherein the isolated nucleic acid includes sequence that is heterologous to the IDE gene allele. In a particular embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 122256 to 122264, wherein the nucleotide at position 122260 is A, T, C or G. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof, that comprises said nucleotide at position 1 22260. For each of these nucleic acid molecules, the coding nucleotide sequence can encode a reporter molecule that is not an IDE protein, or can encode an IDE protein. For each of these embodiments, the promoter can comprise a promoter that is heterologous to an IDE gene, or the promoter can comprise an IDE gene promoter. Also provided are vectors comprising the nucleic acid molecules provided herein, including the nucleic acids describe above. Also provided are cells that can contain one or more of the nucleic acid molecules provided herein, wherein the nucleic acid molecule is heterologous to the cell. Also provided herein are non-human transgenic animals, comprising any one of the nucleic acid molecules provided herein, wherein the nucleic acid molecule is a transgenic element of the animal.

Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260. Each of the polymorphisms listed in Table 14 (as well as any polymorphism in linkage disequilibrium with the polymorphism at position 122260 of SEQ ID NO:484, or in linkage disequilibrium with any polymorphism described herein as being associated with either risk for or protection against diseases, such as AD) is associated with neurodegenerative disease, in particular, AD. The identity of the particular allele or polymorphism at each of the positions listed in Table 14 that associates with neurodegenerative disease can be determined using methods described herein and/or known in the art (e.g., family-based tests for association) for assessing genetic association of a polymorphism with a disease trait. Thus, each of the polymorphisms listed in Table 14 may be assessed in methods of assessing an individual's level of risk for developing a neurodegenerative disease or for determining the occurrence of a neurodegenerative disease in an individual, as described herein, for example, with respect to methods in which the polymorphism at position 122260 is assessed.

Accordingly, also provided herein are isolated nucleic acid molecules, comprising a coding nucleotide sequence operatively linked to a promoter, wherein the coding nucleotide sequence is contained within a nucleotide sequence comprising a portion of an IDE and/or KNSL1 gene allele that comprises a sequence of at least 14, 16, 18, 20, 22, 24, 26, 28, 30, 35, 40, 45, 50, 60, 70, 80, 90 or 100 contiguous nucleotides of an IDE gene allele but does not contain a contiguous sequence of a complete IDE gene allele, wherein the sequence of at least 14 contiguous nucleotides corresponds to a sequence of 14 contiguous nucleotides that includes a nucleotide position selected from 108434; 106995; 98276; 97370; 111972; 110870; 123424; 124692; 130876; 131688; or 134030 of SEQ ID NO:484, or the complement thereof, wherein the nucleotide at the position can be A, T, C or G; and wherein the isolated nucleic acid includes sequence that is heterologous to the IDE and/or KNSL1 gene allele. For each of these nucleic acid molecules, the coding nucleotide sequence can encode a reporter molecule that is not an IDE or KNSL1 protein, or can encode an IDE or KNSL1 protein. For each of these embodiments, the promoter can comprise a promoter that is heterologous to an IDE or KNSL1 gene, or the promoter can comprise an IDE or KNSL1 gene promoter. Also provided are vectors comprising the nucleic acid molecules provided herein, including any of the nucleic acids describe above. Also provided are cells that can contain any one or more of the nucleic acid molecules provided herein, wherein the nucleic acid molecule is heterologous to the cell. Also provided herein are non-human transgenic animals, comprising any one of the nucleic acid molecules provided herein, wherein the nucleic acid molecule is a transgenic element of the animal.

Further provided herein are isolated nucleic acid molecules, comprising a coding nucleotide sequence operatively linked to a promoter, wherein the coding nucleotide sequence is contained within a nucleotide sequence comprising a portion of a KNSL1 gene allele that comprises a sequence of at least 14, 16, 18, 20, 22, 24, 26, 28, 30, 35, 40, 45, 50, 60, 70, 80, 90 or 100 contiguous nucleotides of a KNSL1 gene allele but does not contain a contiguous sequence of a complete KNSL1 gene allele, wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, T, C or G; and wherein the isolated nucleic acid includes sequence that is heterologous to the KNSL1 gene allele. In one embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 132366 to 132374, wherein the nucleotide at position 132370 is A, T, C or G. In another-embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof, that comprises said nucleotide at position 132370. In a particular embodiment, the nucleotide at position 132370 is A, or is T in the complementary position thereof. For each of these embodiments, the coding nucleotide sequence can encode a reporter molecule that is not a KNSL1 protein, or can encode a KNSL1 protein. For each of these embodiments, the promoter can comprise a promoter that is heterologous to a KNSL1 gene, or the promoter can comprise a KNSL1 gene promoter. Also provided are vectors comprising the nucleic acid molecules provided herein, including the nucleic acids describe above. Also provided are cells comprising any one or more of the nucleic acid molecules provided herein, wherein the nucleic acid molecule is heterologous to the cell. Also provided herein are non-human transgenic animals, comprising any one of the nucleic acid molecules provided herein, wherein the nucleic acid molecule is a transgenic element of the animal.

Also provided herein are primers, probes or antisense nucleic acid molecules, comprising a sequence of nucleotides that specifically hybridizes adjacent to, or at, a polymorphic region of a KNSL1 or an IDE gene allele corresponding to: (a) a region that includes position 41014 and/or 41015 of SEQ ID NO:347, or the complementary positions thereof, of a KNSL1 gene allele, or (b) a region that includes position 133354 and/or 133355 of SEQ ID NO:484, or the complementary positions thereof, of a KNSL1 gene allele, or (c) a region that includes position 122260 of SEQ ID NO:484, or the complementary position thereof, of an IDE gene allele. In one embodiment, the sequence of nucleotides specifically hybridizes adjacent to, or at, a sequence of nucleotides, or the complement thereof, of SEQ ID NO:347. In another embodiment, the sequence of nucleotides specifically hybridizes adjacent to, or at, a sequence of nucleotides, or the complement thereof, that includes the nucleotide at position 41014 and/or 41015 of SEQ ID NO:347 and contains the nucleotide sequence AATTT, or the complement thereof, inserted between positions 41014 and 41015, or the complementary positions thereof. In another embodiment, the sequence of nucleotides specifically hybridizes adjacent to, or at, a sequence of nucleotides, or the complement thereof, comprising a sequence of 5, 6, 7, 8 or 9 contiguous nucleotides of SEQ ID NO:347, within the sequence of nucleotides from position 41011 to 41018, wherein the sequence contains the sequence AATTT, or the complement thereof, inserted between the nucleotides at positions 41014 and 41015, or the complementary positions thereof. In another embodiment, the sequence of nucleotides specifically hybridizes adjacent to, or at, a sequence of nucleotides, or the complement thereof, of SEQ ID NO:484. In yet another embodiment, the sequence of nucleotides specifically hybridizes adjacent to, or at, a sequence of nucleotides, or the complement thereof, that includes the nucleotide at position 133354 and/or 133355 of SEQ ID NO:484 and does or does not contain a 6-, 7- or 8-bp polyT sequence, or the complement thereof, inserted between positions 133354 and 133355, or the complementary positions thereof. In another embodiment, the sequence of nucleotides specifically hybridizes adjacent to, or at, a sequence of nucleotides, or the complement thereof, comprising a sequence of 5, 6, 7, 8 or 9 contiguous nucleotides of SEQ ID NO:484,-within the sequence of nucleotides from position 133351 to 133359, wherein the sequence does or does not contain a 6-, 7- or 8-bp polyT sequence, or the complement thereof, inserted between the nucleotides at positions 133354 and 133355, or the complementary positions thereof. In another embodiment, the sequence of nucleotides specifically hybridizes adjacent to, or at, a sequence of nucleotides, or the complement thereof, of SEQ ID NO:484. In yet another embodiment, the sequence of nucleotides specifically hybridizes adjacent to, or at, a sequence of nucleotides, or the complement thereof, that includes a nucleotide at position 122260 of SEQ ID NO:484 wherein the nucleotide is an A or G, or is a T or C in the complementary sequence thereof. In still another embodiment, the sequence of nucleotides specifically hybridizes adjacent to, or at, a sequence of nucleotides, or the complement thereof, comprising a sequence of 5, 6, 7, 8 or 9 contiguous nucleotides of SEQ ID NO:484, within the sequence of nucleotides from position 122256 to 122264, wherein the nucleotide at position 122260 is an A or a G, or is a T or a C in the complementary sequence thereof. In certain embodiments of the primers, probes or antisense molecules provided herein, the sequence of nucleotides contains at least 14 nucleotides but less than 1000 nucleotides. For example, for each of the primers, probes or antisense molecules provided herein, the sequence of nucleotides can contain at least 16, 18, 20, 22, 24, 26, 28, 30, 35, 40, 45, 50, 60, 70, 80, 90, 100, 150, 200, 250, 300, 350, 400, 450, 500, 600, 700, 800, or at least 900 nucleotides.

Also provided herein are kits for use in determining a genotype of a gene allele (such as an IDE or KNSL1 gene allele), comprising at least one container having disposed within at least one primer, probe or antisense nucleic acid molecule provided herein. The kits provided herein can further comprise at least one other container having disposed within at least one primer, probe or antisense nucleic acid molecule which specifically hybridizes adjacent to, or at, a polymorphic region of a gene associated with a disease. In one embodiment, the disease is Alzheimer's disease. In another embodiment, the probe, primer or antisense nucleic acid molecule disposed within the at least one other container specifically hybridizes adjacent to, or at, a polymorphic region of the APOE gene. In other embodiments, the kits provided herein further comprise instructions for genotyping a KNSL1 gene allele.

Also provided herein are solid supports comprising at least one primer, probe or antisense nucleic acid molecule provided herein. The solid supports provided herein can further comprise a probe, primer or antisense nucleic acid molecule that specifically hybridizes adjacent to, or at, a polymorphic region of a gene associated with a disease. In one embodiment, the disease is Alzheimer's disease. In another embodiment, the probe, primer or antisense nucleic acid molecule specifically hybridizes adjacent to, or at, a polymorphic region of the APOE gene. In a particular embodiment, the support is a microarray.

Also provided herein are methods of detecting the presence or absence of a polymorphism of a KNSL1 gene, comprising determining the presence or absence of:
(a) a nucleotide insertion between nucleotides corresponding to nucleotide positions 41014 and 41015 of SEQ ID NO:347, or the complementary positions thereof, or
(b) a nucleotide insertion between nucleotides corresponding to nucleotide positions 133354 and 133355 of SEQ ID NO:484, or the complementary positions thereof, or
(c) a deletion of the nucleotide at a position corresponding to nucleotide position 133354 of SEQ ID NO:484, or at the complementary position thereof. In one embodiment, the method comprises determining the presence or absence of the nucleotide sequence AATTT, or the complement thereof, inserted between nucleotides corresponding to nucleotide positions 41014 and 41015 of SEQ ID NO:347, or the complementary positions thereof. In another embodiment, the method comprises determining the presence or absence of a polyT nucleotide sequence, or the complement thereof, inserted between nucleotides corresponding to nucleotide positions 133354 and 133355 of SEQ ID NO:484, or the complementary positions thereof. In yet another embodiment, the method comprises determining the presence or absence of a 6-, 7-, or 8-bp polyT nucleotide sequence, or the complement thereof, inserted between nucleotides corresponding to nucleotide positions 133354 and 133355 of SEQ ID NO:484, or the complementary positions thereof.

Also provided is a method of detecting the presence or absence of a polymorphism of a gene, comprising determining the identity of a nucleotide at a position corresponding to nucleotide position 132370 of SEQ ID NO:484, or the complement thereof. Similar to the polymorphisms in Table 14 herein that are in linkage disequilibrium with SNP 122260 of SEQ ID NO:484, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484. Also provided is a method of detecting the presence or absence of a polymorphism of a gene, comprising determining the identity of one or more nucleotides at one or more positions corresponding to nucleotide positions 122260, 121239, 120416, 120288, 80752 and 54795 of SEQ ID NO: 484, or the complementary positions thereof. In one embodiment, the identity of a nucleotide at a position corresponding to nucleotide position 122260 of SEQ ID NO:484, or the complement thereof, is determined. In another embodiment, the identity of one or more nucleotides at one or more positions corresponding to nucleotide positions 120416, 120288 and 80752 of SEQ ID NO: 484, or the complementary positions, thereof is (are) determined. In another embodiment, the identities of the nucleotides at each of the positions corresponding to nucleotide positions 120416, 120288 and 80752 of SEQ ID NO: 484, or the complementary positions, thereof are determined. In yet another embodiment, the identity of one or more nucleotides at one or more positions corresponding to nucleotide positions 121239, 120416 and 80752 of SEQ ID NO: 484, or the complementary positions, thereof is (are) determined. In another embodiment, the identities of the nucleotides at each of the positions corresponding to nucleotide positions 121239, 120416 and 80752 of SEQ ID NO: 484, or the complementary positions, thereof are determined. In yet another embodiment, the identity of one or more nucleotides at one or more positions corresponding to nucleotide positions 122260, 120416, 120288, 80752 and 54795 of SEQ ID NO: 484, or the complementary positions thereof, is (are) determined. In a particular embodiment, the identities of the nucleotides at each of the positions corresponding to nucleotide positions 122260, 120416, 120288, 80752 and 54795 of SEQ ID NO: 484, or the complementary positions thereof, are determined. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260. Accordingly, also provided is a method of detecting the presence or absence of a polymorphism of a gene, comprising determining the identity of one or more nucleotides at one or more positions corresponding to nucleotide positions 108434; 106995; 98276; 97370; 111972; 110870; 123424; 124692; 130876; 131688; or 134030 of SEQ ID NO:484, or the complementary positions thereof. In a particular embodiment of the methods provided herein, the identity of the nucleotide(s) is determined in nucleic acid obtained from an individual who has or exhibits a characteristic of a neurodegenerative disease or who has a family member who has a neurodegenerative disease, such as among others Alzheimer's Disease (AD).

Also provided herein are methods for assessing an individual's level of risk for developing a neurodegenerative disease or for determining the occurrence of a neurodegenerative disease in an individual, comprising:
assessing in a nucleic acid sample obtained from an individual the presence of one or more polymorphisms of chromosome 10 selected from the group consisting of:
(a) a nucleotide insertion between nucleotides corresponding to nucleotide positions 41014 and 41015 of SEQ ID NO:347, or the complementary positions thereof;
(b) a nucleotide insertion between nucleotides corresponding to nucleotide positions 133354 and 133355 of SEQ ID NO:484, or the complementary positions thereof; and
(c) a nucleotide at one or more positions corresponding to nucleotide positions 132370, 122260, 121239, 120416, 120288, 80752 and 54795 of SEQ ID NO: 484, or the complementary positions thereof;

wherein the presence of the polymorphism is indicative of risk for or protection against a neurodegenerative disease. The method can further comprise determining if the individual is homozygous for the polymorphism. In one embodiment, the method comprises detecting the presence or absence of a nucleotide insertion between nucleotides corresponding to nucleotide positions 41014 and 41015 of SEQ ID NO:347, or the complementary positions thereof, such as the nucleotide sequence AATTT, or the complement thereof. In this embodiment, the presence of the respective polymorphism(s) is indicative of risk for a neurodegenerative disease, e.g., Alzheimer's Disease. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260. Similar to the polymorphisms in Table 14 herein that are in linkage disequilibrium with SNP 122260 of SEQ ID NO:484, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484. Likewise, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

In another embodiment, the method comprises detecting the presence or absence of a nucleotide insertion between nucleotides corresponding to nucleotide positions 133354 and 133355 of SEQ ID NO:484, or the complementary positions thereof, such as a polyT nucleotide sequence or the nucleotide sequence TTTTTTT, or the complements thereof. In this embodiment, the presence of the respective polymorphism(s) is indicative of risk for a neurodegenerative disease, e.g., Alzheimer's Disease. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

In yet another embodiment, the method comprises assessing the presence of a polymorphism at one or more positions corresponding to nucleotide positions 132370, 122260, 121239, 120416, 120288, 80752 and 54795 of SEQ ID NO: 484, or the complementary positions thereof. In a particular embodiment, the method comprises assessing the presence of a polymorphism at a position corresponding to nucleotide position 122260 of SEQ ID NO:484, or the complementary position thereof. In one embodiment, the presence of a G at a position corresponding to nucleotide position 122260 of SEQ ID NO:484, or a C at a position corresponding to the complementary position thereof, is assessed. In this 37481-3308B embodiment, the presence of the respective polymorphism(s) is indicative of risk for a neurodegenerative disease, e.g., Alzheimer's Disease. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260.

In another embodiment, the presence of an A at a position corresponding to nucleotide position 122260 of SEQ ID NO:484, or a T at a position corresponding to the complementary position thereof, is assessed. In this embodiment, the presence of the respective polymorphism(s) is indicative of protection against a neurodegenerative disease, e.g., Alzheimer's Disease. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 1,22260.

In yet another embodiment, the method comprises assessing the presence of a polymorphism at a position corresponding to nucleotide position 132370 of SEQ ID NO:484, or the complementary position thereof. For example, the presence of an A at a position corresponding to nucleotide position 132370 of SEQ ID NO:484, or a T at a position corresponding to the complementary position thereof, is assessed. In this embodiment, the presence of the respective polymorphism(s) is indicative of risk for a neurodegenerative disease, e.g., Alzheimer's Disease. Similar to the polymorphisms in Table 14 herein that are in linkage disequilibrium with SNP 122260 of SEQ ID NO:484, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484.

In yet another embodiment, the method comprises assessing the presence of one or more of the following nucleotides:
(a) an A at a position corresponding to nucleotide position 121239 of SEQ ID NO:484, or a T at a position corresponding to the complementary position thereof;
(b) an A at a position corresponding to nucleotide position 120416 of SEQ ID NO:484, or a T at a position corresponding to the complementary position thereof;
(c) a G at a position corresponding to nucleotide position 120288 of SEQ ID NO:484, or a C at a position corresponding to the complementary position thereof;
(d) an A at a position corresponding to nucleotide position 80752 of SEQ ID NO:484, or a T at a position corresponding to the complementary position thereof; and
(e) a G at a position corresponding to nucleotide position 54795 of SEQ ID NO:484, or a C at a position corresponding to the complementary position thereof.

In another embodiment, the method comprises assessing the presence of a polymorphism at each of the positions corresponding to nucleotide positions 120416, 120288 and 80752 of SEQ ID NO: 484, or the complementary positions thereof. For example, the method can comprise assessing the presence of an A at a position corresponding to nucleotide position 120416 of SEQ ID NO:484, or a T at a position corresponding to the complementary position thereof; an A at a position corresponding to nucleotide position 120288 of SEQ ID NO:484, or a T at a position corresponding to the complementary position thereof; and an A at a position corresponding to nucleotide position 80752 of SEQ ID NO:484, or a T at a position corresponding to the complementary position thereof. In this embodiment, the presence of the respective polymorphism(s) is indicative of risk for a neurodegenerative disease, e.g., Alzheimer's Disease.

In yet another embodiment, the method comprises assessing the presence of a polymorphism at each of the positions corresponding to nucleotide positions 121239, 120416 and 80752 of SEQ ID NO: 484, or the complementary positions thereof. For example, the method can comprise assessing the-presence of a C at a position corresponding to nucleotide position 121239 of SEQ ID NO:484, or a G at a position corresponding to the complementary position thereof; an A at a position corresponding to nucleotide position 120416 of SEQ ID NO:484, or a T at a position corresponding to the complementary position thereof; and an A at a position corresponding to nucleotide position 80752 of SEQ ID NO:484, or a T at a position corresponding to the complementary position thereof. In this embodiment, the presence of the respective polymorphism(s) is indicative of risk for a neurodegenerative disease, e.g., Alzheimer's Disease.

In yet another embodiment, the method comprises assessing the presence of a polymorphism at each of the corresponding to nucleotide positions 122260, 120416, 120288, 80752 and 54795 of SEQ ID NO: 484, or the complementary positions thereof. For example, the method can comprise assessing the presence of a G at a position corresponding to nucleotide position 122260 of SEQ ID NO:484, or a C at a position corresponding to the complementary position thereof; an A at a position corresponding to nucleotide position 120416 of SEQ ID NO:484, or a T at a position corresponding to the complementary position thereof; a G at a position corresponding to nucleotide position 120288 of SEQ ID NO:484, or a C at a position corresponding to the complementary position thereof; and an A at a position corresponding to nucleotide position 80752 of SEQ ID NO:484, or a T at a position corresponding to the complementary position thereof; and a G at a position corresponding to nucleotide position 54795 of SEQ ID NO:484, or a C at a position corresponding to the complementary position thereof. In this embodiment, the presence of the respective polymorphism(s) is indicative of risk for a neurodegenerative disease, e.g.,-Alzheimer's Disease.

Alternatively, the method can comprise assessing the presence of an A at a position corresponding to nucleotide position 122260 of SEQ ID NO:484, or a T at a position corresponding to the complementary position thereof; an A at a position corresponding to nucleotide position 120416 of SEQ ID NO:484, or a T at a position corresponding to the complementary position thereof; a G at a position corresponding to nucleotide position 120288 of SEQ ID NO:484, or a C at a position corresponding to the complementary position thereof; and an A at a position corresponding to nucleotide position 80752 of SEQ ID NO:484, or a T at a position corresponding to the complementary position thereof; and a G at a position corresponding to nucleotide position 54795 of SEQ ID NO:484, or a C at a position corresponding to the complementary position thereof. In this embodiment, the presence of the respective polymorphism(s) is indicative of protection against a neurodegenerative disease, e.g., Alzheimer's Disease.

Methods of Screening for an Agent that Modulates the Expression and/or Activity of IDE

Also provided herein are methods of screening for an agent that modulates the expression and/or activity of IDE, comprising:
assessing the effect of a test agent on the expression of a coding nucleotide sequence operatively linked to a promoter, wherein the coding nucleotide sequence is contained within a nucleotide sequence comprising at least a portion of the IDE gene that comprises a sequence of at least 14, 16, 18, 20, 22, 24, 26, 28, 30, 35, 40, 45, 50, 60, 70, 80, 90 or 100 contiguous nucleotides of an IDE gene allele,
wherein the sequence of at least 14 contiguous nucleotides corresponds to a sequence of at least 14 contiguous nucleotides that includes the nucleotide at position 122260 of SEQ ID NO:484, or the complement thereof, wherein the nucleotide at position 122260 is replaced with a G, T or C, or is replaced with a C, A or G in a complementary sequence thereof; and
identifying a test agent as an agent that modulates the expression and/or activity of IDE if it has an effect on expression of the coding nucleotide sequence. In one embodiment, the sequence of 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 122256 to 122264, except that the nucleotide at position 122260 is replaced with a G, T or C, or is replaced with a C, A or G in the complement thereof. In another embodiment, the sequence of 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in the complement thereof. In a particular embodiment, the nucleotide at position 122260 is replaced with a G, or is replaced with a C in a complementary sequence thereof. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260.

Also provided are methods of screening for an agent that modulates the expression and/or activity of IDE, comprising:
assessing the effect of a test agent on the expression of a coding nucleotide sequence operatively linked to a promoter, wherein the coding nucleotide sequence is contained within a nucleotide sequence comprising a portion of the IDE gene that comprises a sequence of at least 14, 16, 18, 20, 22, 24, 26, 28, 30, 35, 40, 45, 50, 60, 70, 80, 90 or 100 contiguous nucleotides of an IDE gene allele but that is not a contiguous sequence of a complete IDE allele;
wherein, the sequence of at least 14 contiguous nucleotides corresponds to a sequence of at least 14 contiguous nucleotides that includes the nucleotide at position 122260 of SEQ ID NO:484, or the complement thereof; and
identifying a test agent as an agent that modulates the expression and/or activity of IDE if it has an effect on expression of the coding nucleotide sequence. In one embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO: 484, or the complement thereof, within the sequence of nucleotides from position 122256 to 122264. In another embodiment, the sequence of 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof. The coding nucleotide sequence, promoter and portion of the IDE gene can be contained in a nucleotide sequence that includes sequence that is heterologous to the IDE gene.

For each of these embodiments, the coding sequence of nucleotides can encode an IDE protein or a reporter molecule. Likewise, the assessing step can comprise assessing the effect on expression of the coding sequence of nucleotides in a cell or non-human animal that comprises the coding sequence of nucleotides operatively linked to a promoter and contained within a nucleotide sequence comprising the portion of the IDE gene. The cell can be a recombinant cell and the non-human animal can be a transgenic animal. In certain embodiments, the coding sequence operatively linked to a promoter and contained within a nucleotide sequence comprising the portion of the IDE gene is heterologous to the cell or is a transgenic element in the transgenic animal.

For each of these embodiments, the promoter can comprise an IDE gene promoter. Likewise, a test agent is identified as an agent that modulates the expression and/or activity of IDE if increases or decreases the level of expression of the coding sequence of nucleotides. For example, a test agent can be identified as an agent that modulates the expression and/or activity of IDE if it alters the level of expression of the coding sequence of nucleotides such that it is substantially similar to the level of expression of a coding sequence of nucleotides operatively linked to a promoter and contained within a nucleotide sequence comprising the portion of the IDE gene wherein the nucleotide at position 122260 is an A, or wherein the nucleotide at position 122260 is a T in the complementary sequence thereof. For each of these embodiment, assessing can comprise determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides. In addition, because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260.

Method of Screening for an Agent that Modulates the Expression and/or Activity of KNSL1

Also provided herein are methods of screening for an agent that modulates the-expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 50, 60, 70, 80, 90 or 100 contiguous nucleotides of a KNSL1 allele,
wherein the sequence of at least 50 contiguous nucleotides corresponds to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof; and
identifying a test agent as an agent that modulates the expression and/or activity of KNSL1 if it has an effect on expression of the coding nucleotide sequence. In one embodiment, the sequence of at least 50, 60, 70, 80, 90 or 100 contiguous nucleotides comprises a 6-, 7- or 8-bp polyT sequence, or the complement thereof, inserted between nucleotides at positions 133354 and 133355, or the complementary positions thereof. In another embodiment, the sequence of at least 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133351 to 133358, and one or more nucleotides inserted between positions 133354 and 133355, or the complementary positions thereof. In another embodiment, the sequence of at least 50, 60, 70, 80, 90 or 100 contiguous nucleotides comprises a sequence of at least 50 contiguous nucleotides of SEQ ID NO:484, or the complement thereof, and one or more nucleotides inserted between positions 133354 and 133355, or the complementary positions thereof. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

Also provided herein are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 50, 60, 70, 80, 90 or 100 contiguous nucleotides of a KNSL1 gene allele,
wherein the sequence of at least 50 contiguous nucleotides corresponds to a sequence of at least 50 contiguous nucleotides that includes nucleotide position 133354 of SEQ ID NO:484, or the complement thereof, except that the nucleotide at position 133354, or the complementary position thereof, is deleted; and
identifying a test agent as an agent that modulates the expression and/or activity of KNSL1 if it has an effect on expression of the coding nucleotide sequence. In one embodiment, the sequence of 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133351 to 133357, except that the nucleotide at position 133354, or the complementary position thereof, is deleted. In another embodiment, the sequence of 50, 60, 70, 80, 90 or 100 contiguous nucleotides comprises a sequence of at least 50 contiguous nucleotides of SEQ ID NO:484, or the complement thereof, except that the nucleotide at position 133354, or the complementary position thereof, is deleted.

Also provided herein are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 50, 60, 70, 80, 90 or 100 contiguous nucleotides of a KNSL1 gene allele but that is not a contiguous sequence of a complete KNSL1 allele;
wherein the sequence of at least 50 contiguous nucleotides corresponds to a sequence of-at least 50 contiguous nucleotides that includes nucleotide position 133354 and/or 133355, or the complement thereof, and contains or does not contain one or more nucleotides inserted between nucleotides 133354 and 133355; and
identifying a test agent as an agent that modulates the expression and/or activity of KNSL1 if it has an effect on expression of the coding nucleotide sequence. In one embodiment, the sequence of at least 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133350 to 133359, and contains or does not contain one or more nucleotides inserted between nucleotides 133354 and 133355. In another embodiment, the sequence of at least 50, 60, 70, 80, 90 or 100 contiguous nucleotides comprises a sequence of at least 50 contiguous nucleotides of SEQ ID NO:484, or the complement thereof, and contains or does not contain one or more nucleotides inserted between nucleotides 133354 and 133355. In a particular embodiment, the coding nucleotide sequence, promoter and portion of the KNSL1 gene are contained in a nucleotide sequence that includes sequence that is heterologous to the KNSL1 gene. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

For each of these embodiments, the coding sequence of nucleotides can encode an KNSL1 protein or a reporter molecule. Likewise, the assessing step can comprise assessing the effect on expression of the coding sequence of nucleotides in a cell or non-human animal that comprises the coding sequence of nucleotides operatively linked to a promoter and contained within a nucleotide sequence comprising the portion of the KNSL1 gene. The cell can be a recombinant cell and the non-human animal can be a transgenic animal. In certain embodiments, the coding sequence operatively linked to a promoter and contained within a nucleotide sequence comprising the portion of the KNSL1 gene is heterologous to the cell or is a transgenic element in the transgenic animal. In a particular embodiment, the promoter comprises a KNSL1 gene promoter.

In each of these embodiments, a test agent can be identified as an agent that modulates the expression and/or activity of KNSL1 if increases or decreases the level of expression of the coding sequence of nucleotides. In other embodiments, a test agent can be identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the sequence does not contain one or more nucleotides inserted between nucleotide positions 133354 and 133355. In a particular embodiment, the one or more nucleotides inserted between nucleotide positions 133354 and 133355 is a 7-bp polyT sequence, or the complement thereof inserted between the complementary positions thereof. For each of these embodiment, the assessing can comprise determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

Also provided are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele,
wherein the sequence of at least 14 contiguous nucleotides corresponds to a sequence of at least 14 contiguous nucleotides that includes nucleotide position 41014 and/or 41015 of SEQ ID NO:347, or the complement thereof, and one or more nucleotides inserted between nucleotides at positions 41014 and 41015, or the complementary positions thereof; and
identifying a test agent as an agent that modulates the expression and/or activity of KNSL1 if it has an effect on expression of the coding nucleotide sequence. In one embodiment, the sequence of at least 14, 16, 18, 20, 22, 24, 26, 28, 30, 35, 40, 45, 50, 60, 70, 80, 90 or 100 contiguous nucleotides comprises the nucleotide sequence AATTT, or the complement thereof, inserted between nucleotides at positions 41014 and 41015 of SEQ ID NO:347, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:347, or the complement thereof, within the sequence of nucleotides from position 41011 to 41018, and one or more nucleotides inserted between positions 41011 and 41018, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:347, or the complement thereof, and one or more nucleotides inserted between positions 41011 and 41018, or the complementary positions thereof.

Also provided are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele but that is not a contiguous sequence of a complete KNSL1 allele;
wherein the sequence of at least 14 contiguous nucleotides corresponds to a sequence of at least 14 contiguous nucleotides that includes nucleotide position 41014 and/or 41015, or the complement thereof, and contains or does not contain one or more nucleotides inserted between nucleotides 41014 and 41015; and
identifying a test agent as an agent that modulates the expression and/or activity of KNSL1 if it has an effect on expression of the coding nucleotide sequence. In one embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:347, or the complement thereof, within the sequence of nucleotides from position 41010 to 41019, and contains or does not contain one or more nucleotides inserted between nucleotides 41014 and 41015, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:347, or the complement thereof, and contains or does not contain one or more nucleotides inserted between nucleotides 41014 and 41015, or the complementary positions thereof. In a particular embodiment, the coding nucleotide sequence, promoter and portion of the KNSL1 gene are contained in a nucleotide sequence that includes sequence that is heterologous to the KNSL1 gene.

For each of these embodiments, the coding sequence of nucleotides can encode an KNSL1 protein or a reporter molecule. Likewise, the assessing step can comprise assessing the effect on expression of the coding sequence of nucleotides in a cell or non-human animal that comprises the coding sequence of nucleotides operatively linked to a promoter and contained within a nucleotide sequence comprising the portion of the KNSL1 gene. The cell can be a recombinant cell and the non-human animal can be a transgenic animal. In certain embodiments, the coding sequence operatively linked to a promoter and contained within a nucleotide sequence comprising the portion of the KNSL1 gene is heterologous to the cell or is a transgenic element in the transgenic animal. In a particular embodiment, the promoter comprises a KNSL1 gene promoter.

In each of these embodiments, a test agent can be identified as an agent that modulates the expression and/or activity of KNSL1 if increases or decreases the level of expression of the coding sequence of nucleotides. In other embodiments, a test agent can be identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the sequence does not contain one or more nucleotides inserted between nucleotide positions 41014 and 41015. In a particular embodiment, the one or more nucleotides inserted between nucleotide positions 41014 and 41015 comprises the sequence AATTT, or the complement thereof inserted between the complementary positions thereof. For each of these embodiment, the assessing can comprise determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

Also provided are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele,
wherein, the sequence of at least 14 contiguous nucleotides corresponds to a sequence of 14 contiguous nucleotides that includes nucleotide position 132370 of SEQ ID NO:484, or the complement thereof, except that the nucleotide at position 132370 is replaced with an A or is replaced with a T in the complement thereof; and
identifying a test agent as an agent that modulates the expression and/or activity of KNSL1 if it has an effect on expression of the coding nucleotide sequence. Also provided are methods of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele but that is not a contiguous sequence of a complete KNSL1 allele;
wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, G, T or C; and
identifying a test agent as an agent that modulates the expression and/or activity of KNSL1 if it has an effect on expression of the coding nucleotide sequence. For each of these methods, the sequence of at least 14, 16, 18,-20, 22, 24, 26, 28, 30, 35, 40, 45, 50, 60, 70, 80, 90 or 100contiguous nucleotides can comprise a sequence of 5, 6, 7, 8 or 9 contiguous nucleotides of SEQ ID NO: 484, or the complement thereof, within the sequence of nucleotides from position 132366 to132374, that comprises said nucleotide at position 132370. In other embodiments, the sequence of at least 14, 16, 18, 20, 22, 24, 26, 28, 30, 35, 40, 45, 50, 60, 70, 80, 90 or 100 contiguous nucleotides is a sequence of at least 14 contiguous nucleotides of SEQ ID NO: 484, or the complement thereof, that comprises said nucleotide position at 132370. In a particular embodiment, the coding nucleotide sequence, promoter and portion of the KNSL1 gene are contained in a nucleotide sequence that includes sequence that is heterologous to the KNSL1 gene. Similar to the polymorphisms in Table 14 herein that are in linkage disequilibrium with SNP 122260 of SEQ ID NO:484, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484.

For each of these embodiments, the coding sequence of nucleotides can encode an KNSL1 protein or a reporter molecule. Likewise, the assessing step can comprise assessing the effect on expression of the coding sequence of nucleotides in a cell or non-human animal that comprises the coding sequence of nucleotides operatively linked to a promoter and contained within a nucleotide sequence comprising the portion of the KNSL1 gene. The cell can be a recombinant cell and the non-human animal can be a transgenic animal. In certain embodiments, the coding sequence operatively linked to a promoter and contained within a nucleotide sequence comprising the portion of the KNSL1 gene is heterologous to the cell or is a transgenic element in the transgenic animal. In a particular embodiment, the promoter comprises a KNSL1 gene promoter.

In each of these embodiments, a test agent can be identified as an agent that modulates the expression and/or activity of KNSL1 if increases or decreases the level of expression of the coding sequence of nucleotides. In other embodiments, a test agent is identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the nucleotide at position 132370 is a G or wherein the nucleotide at position 132370 is a C in the complementary sequence thereof. For each of these embodiment, the assessing can comprise determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

Methods of Screening for an Agent that Modulates a Biological Event Characteristic of a Neurodegenerative Disease

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding IDE operatively linked to a promoter,
wherein the coding nucleotide sequence is contained within a nucleotide sequence comprising at least a portion of the IDE gene that comprises a sequence of at least 14, 16, 18, 20, 22, 24, 26, 28, 30, 35, 40, 45, 50, 60, 70, 80, 90 or 100 contiguous nucleotides of an IDE gene allele,
wherein the sequence of at least 14 contiguous nucleotides corresponds to a sequence of at least 14 contiguous nucleotides that includes the nucleotide at position 122260 of SEQ ID NO:484, or the complement thereof, wherein the nucleotide at position 122260 is replaced with a G, T or C, or is replaced with a C, A or G in a complementary sequence thereof; and
identifying a test agent as an agent that modulates a biological event characteristic of a neurodegenerative disease if it has an effect on the biological event characteristic of a neurodegenerative disease. In one embodiment, the sequence of 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 122256 to 122264, except that the nucleotide at position 122260 is replaced with a G, T or C, or is replaced with a C, A or G in the complement thereof. In another embodiment, the sequence of 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in the complement thereof. In a particular embodiment, the nucleotide at position 122260 is replaced with a G, or is replaced with a C in a complementary sequence thereof.

Also provided are methods of screening for an agent that modulates a biological event characteristic of a neurodegenerative disease (e.g., Alzheimer's disease), comprising:
assessing the effect of a test agent on a biological event characteristic of a neurodegenerative disease exhibited by a cell or animal that comprises a sequence of nucleotides encoding IDE operatively linked to a promoter,
wherein the coding nucleotide sequence is contained within a nucleotide sequence comprising a portion of the IDE gene that comprises a sequence of at least 14, 16, 18, 20, 22, 24, 26, 28, 30, 35, 40, 45, 50, 60, 70, 80, 90 or 100 contiguous nucleotides of an IDE gene allele but that is not a contiguous sequence of a complete IDE allele, and
wherein, the sequence of at least 14 contiguous nucleotides corresponds to a sequence of at least 14 contiguous nucleotides that includes the nucleotide at position 122260 of SEQ ID NO:484, or the complement thereof; and
identifying a test agent as an agent that modulates a biological event characteristic of a neurodegenerative disease if it has an effect on the biological event characteristic of a neurodegenerative disease. In one embodiment, the sequence of 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 122256 to 122264. In another embodiment, the sequence of 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:484, or the complement thereof.

For each of these embodiments, the coding nucleotide sequence, promoter and portion of the IDE gene are contained in a nucleotide sequence that includes sequence that is heterologous to the IDE gene. Likewise, the nucleotide sequence comprising a portion of the IDE gene can be heterologous to the cell or animal. The cell can be a recombinant cell, the animal can be a non-human transgenic animal. For each of these embodiments, the promoter can comprise an IDE gene promoter. Likewise, the biological event can be the level of an Aβ peptide in the cell, extracellular medium or animal. For each of these embodiments, because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260.

Also provide are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 50, 60, 70, 80, 90 or 100 contiguous nucleotides of a KNSL1 gene allele, and
wherein the sequence of at least 50 contiguous nucleotides corresponds to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof; and
identifying a test agent as an agent that modulates a biological event characteristic of a neurodegenerative disease if it has an effect on the biological event characteristic of a neurodegenerative disease. In one embodiment, the sequence of at least 50, 60, 70, 80, 90 or 100 contiguous nucleotides comprises a 6-, 7- or 8-bp polyT sequence, or the complement thereof, inserted between nucleotides at positions 133354 and 133355, or the complementary positions thereof. In another embodiment, the sequence of at least 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133351 to 133358, and one or more nucleotides inserted between positions 133354 and 133355, or the complementary positions thereof. In another embodiment, the sequence of at least 50, 60, 70, 80, 90 or 100 contiguous nucleotides comprises a sequence of at least 50 contiguous nucleotides of SEQ ID NO:484, or the complement thereof, and one or more nucleotides inserted between positions 133354 and 133355, or the complementary positions thereof. In yet another embodiment, the sequence of at least 50, 60, 70, 80, 90 or 100 contiguous nucleotides comprises a 7-bp polyT sequence, or the complement thereof, inserted between nucleotides at positions 133354 and 133355, or the complementary positions thereof. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 50, 60, 70, 80, 90 or 100 contiguous nucleotides of a KNSL1 gene allele, and
wherein the sequence of at least 50 contiguous nucleotides corresponds to a sequence of at least 50 contiguous nucleotides that includes nucleotide position 133354 of SEQ ID NO:484, or the complement thereof, except that the nucleotide at position 133354, or the complementary position thereof, is deleted; and
identifying a test agent as an agent that modulates a biological event characteristic of a neurodegenerative disease if it has an effect on the biological event characteristic of a neurodegenerative disease. In one embodiment, the sequence of at least 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133351 to 133357, except that the nucleotide at position 133354, or the complementary position thereof, is deleted. In another embodiment, the sequence of at least 50, 60, 70, 80, 90 or 100 contiguous nucleotides comprises a sequence of at least 50 contiguous nucleotides of SEQ ID NO:484, or the complement thereof, except that the nucleotide at position 133354, or the complementary position thereof, is deleted.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 50, 60, 70, 80, 90 or 100 contiguous nucleotides of a KNSL1 gene allele but that is not a contiguous sequence of a complete KNSL1 allele, and
wherein the sequence of at least 50 contiguous nucleotides corresponds to a sequence of at least 50 contiguous nucleotides that includes nucleotide position 133354 and/or 133355, or the complement thereof, and contains or does not contain one or more nucleotides inserted between nucleotides 133354 and 133355; and
identifying a test agent as an agent that modulates a biological event characteristic of a neurodegenerative disease if it has an effect on the biological event characteristic of a neurodegenerative disease. In one embodiment, the sequence of at least 50, 60, 70, 80, 90 or 100 contiguous nucleotides comprises a sequence of 5, 6, 7, 8 or 9 contiguous nucleotides of SEQ ID NO:484, or the complement thereof, within the sequence of nucleotides from position 133350 to 133359, and contains or does not contain one or more nucleotides inserted between nucleotides 133354 and 133355. In another embodiment, the sequence of at least 50, 60, 70, 80, 90 or 100 contiguous nucleotides comprises a sequence of at least 50 contiguous nucleotides of SEQ ID NO:484, or the complement thereof, and contains or does not contain one or more nucleotides inserted between nucleotides 133354 and 133355. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

For each of these embodiments, the coding nucleotide sequence, promoter and portion of the KNSL1 gene are contained in a nucleotide sequence that includes sequence that is heterologous to the KNSL1 gene. Likewise, the nucleotide sequence comprising a portion of the KNSL1 gene can be heterologous to the cell or animal. The cell can be a recombinant cell, the animal can be a non-human transgenic animal. For each of these embodiments, the promoter can comprise an KNSL1 gene promoter. Likewise, the biological event can be the level of an Aβ peptide in the cell, extracellular medium or animal. In a particular embodiment, the neurodegenerative disease is Alzheimer's disease.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele, and
wherein the sequence of at least 14 contiguous nucleotides corresponds to a sequence of at least 14 contiguous nucleotides that includes nucleotide position 41014 and/or 41015 of SEQ ID NO:347, or the complement thereof, and one or more nucleotides inserted between nucleotides at positions 41014 and 41015, or the complementary positions thereof; and
identifying a test agent as an agent that modulates a biological event characteristic of a neurodegenerative disease if it has an effect on the biological event characteristic of a neurodegenerative disease. In one embodiment, the sequence of at least 14, 16, 18, 20, 22, 24, 26, 28, 30, 35, 40, 45, 50, 60, 70, 80, 90 or 100 contiguous nucleotides comprises the nucleotide sequence AATTT, or the complement thereof, inserted between nucleotides at positions 41014 and 41015, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:347, or the complement thereof, within the sequence of nucleotides from position 41011 to 41018, and one or more nucleotides inserted between positions 41011 and 41018, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:347, or the complement thereof, and one or more nucleotides inserted between positions 41011 and 41018, or the complementary positions thereof.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele but that is not a contiguous sequence of a complete KNSL1 allele, and
wherein the sequence of at least 14 contiguous nucleotides corresponds to a sequence of at least 14 contiguous nucleotides that includes nucleotide position 41014 and/or 41015, or the complement thereof, and contains or does not contain one or more nucleotides inserted between nucleotides 41014 and 41015; and
identifying a test agent as an agent that modulates a biological event characteristic of a neurodegenerative disease if it has an effect on the biological event characteristic of a neurodegenerative disease. In one, embodiment, the sequence of at least 14, 16, 18, 20, 22, 24, 26, 28, 30, 35, 40, 45, 50, 60, 70, 80, 90 or 100 contiguous nucleotides comprises a sequence of 5, 6, 7, 8 or 9 contiguous nucleotides of SEQ ID NO:347, or the complement thereof, within the sequence of nucleotides from position 41010 to 41019, and contains or does not contain one or more nucleotides inserted between nucleotides 41014 and 41015, or the complementary positions thereof. In another embodiment, the sequence of at least 14, 16, 18, 20, 22, 24, 26, 28, 30, 35, 40, 45, 50, 60, 70, 80, 90 or 100 contiguous nucleotides comprises a sequence of at least 14 contiguous nucleotides of SEQ ID NO:347, or the complement thereof, and contains or does not contain one or more nucleotides inserted between nucleotides 41014 and 41015, or the complementary positions thereof.

For each of these embodiments, the coding nucleotide sequence, promoter and portion of the KNSL1 gene are contained in a nucleotide sequence that includes sequence that is heterologous to the KNSL1 gene. Likewise, the nucleotide sequence comprising a portion of the KNSL1 gene can be heterologous to the cell or animal. The cell can be a recombinant cell, the animal can be a non-human transgenic animal. For each of these embodiments, the promoter can comprise an KNSL1 gene promoter. Likewise, the biological event can be the level of an Aβ peptide in the cell, extracellular medium or animal. In a particular embodiment, the neurodegenerative disease is Alzheimer's disease.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele, and
wherein the sequence of at least 14 contiguous nucleotides corresponds to a sequence of at least 14 contiguous nucleotides that includes nucleotide position 132370 of SEQ ID NO:484, or the complement thereof, except that the nucleotide at position 132370 is replaced with an A or is replaced with a T in the complement thereof; and
identifying a test agent as an agent that modulates a biological event characteristic of a neurodegenerative disease if it has an effect on the biological event characteristic of a neurodegenerative disease.

Also provided are methods of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 14, 16, 18, 20, 22, 24, 26, 28, 30, 35, 40, 45, 50, 60, 70, 80, 90 or 100 contiguous nucleotides of a KNSL1 gene allele but that is not a contiguous sequence of a complete KNSL1 allele;

wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, G, T or C; and
identifying a test agent as an agent that modulates a biological event characteristic of a neurodegenerative disease if it has an effect on the biological event characteristic of a neurodegenerative disease. For each of these methods, the sequence of at least 14, 16, 18, 20, 22, 24, 26, 28, 30, 35, 40, 45, 50, 60, 70, 80, 90 or 100 contiguous nucleotides can comprise a sequence of 5, 6, 7, 8 or 9 contiguous nucleotides of SEQ ID NO: 484, or the complement thereof, within the sequence of nucleotides from position 132366 to 132374, that comprises said nucleotide at position 132370. In another embodiment, the sequence of at least 14, 16, 18, 20, 22, 24, 26, 28, 30, 35, 40, 45, 50, 60, 70, 80, 90 or 100 contiguous nucleotides is a sequence of at least 14 contiguous nucleotides of SEQ ID NO: 484, or the complement thereof, that comprises said nucleotide position at 132370. Similar to the polymorphisms in Table 14 herein that are in linkage disequilibrium with SNP 122260 of SEQ ID NO:484, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484.

For each of these embodiments, the coding nucleotide sequence, promoter and portion of the KNSL1 gene are contained in a nucleotide sequence that includes sequence that is heterologous to the KNSL1 gene. Likewise, the nucleotide sequence comprising a portion of the KNSL1 gene can be heterologous to the cell or animal. The cell can be a recombinant cell, the animal can be a non-human transgenic animal. For each of these embodiments, the promoter can comprise an KNSL1 gene promoter. Likewise, the biological event can be the level of an AA8 peptide in the cell, extracellular medium or animal. In a particular embodiment, the neurodegenerative disease is Alzheimer's disease.

Also provided are methods for determining a predisposition for or the occurrence of neurodegenerative disease in a subject, comprising:
the step of detecting in a target nucleic acid obtained from the subject the presence or absence of an allelic variant of an IDE gene corresponding to nucleotide C at a position complementary to position 122260 of SEQ ID NO:484, wherein the presence of said allelic variant is indicative of a predisposition for or the occurrence of neurodegenerative disease. Also provided are methods for determining a level of risk for the occurrence of neurodegenerative disease in a subject, comprising:
the step of detecting in a target nucleic acid obtained from the subject the presence or absence of an allelic variant of an IDE gene corresponding to nucleotide C at a position complementary to position 122260 of SEQ ID NO:484, wherein the presence of said allelic variant is indicative of an increased risk for the occurrence of neurodegenerative disease compared to a subject not having said allelic variant. Because position 122260 of SEQ ID NO:484 is in linkage disequilibrium (LD) with each of the positions set forth in Table 14, the polymorphisms at each of these positions in Table 14 may be assessed in place of assessing the polymorphism at position 122260 for all methods and applications provided herein assessing the polymorphism at position 122260.

Also provided are methods for determining a predisposition for or the occurrence of neurodegenerative disease in a subject, comprising:
the step of detecting in a target nucleic acid obtained from the subject the presence or absence of an allelic variant of a KNSL1 gene corresponding to an insertion of -AATTT- between nucleotides at positions 41014 and 41015 of SEQ ID NO:347, wherein the presence of said allelic variant is indicative of a predisposition for or the occurrence of neurodegenerative disease.

Also provided are methods for determining a level of risk for the occurrence of neurodegenerative disease in a subject, comprising:
the step of detecting in a target nucleic acid obtained from the subject the presence or absence of an allelic variant of a KNSL1 gene corresponding to an insertion of -AATTT- between nucleotides at positions 41014 and 41015 of SEQ ID NO:347, wherein the presence of said allelic variant is indicative of an increased level of risk for the occurrence of neurodegenerative disease compared to a subject not having said allelic variant.

Also provided are methods for determining a predisposition for or the occurrence of neurodegenerative disease in a subject, comprising:
the step of detecting in a target nucleic acid obtained from the subject the presence or absence of an allelic variant of a KNSL1 gene corresponding to an insertion of a 7 base pair poly-T sequence corresponding to -TTTTTTT- between positions 133354-133355 of SEQ ID NO:484, wherein the presence of said allelic variant is indicative of a predisposition for or the occurrence of neurodegenerative disease. Also provided are methods for determining a level of risk for the occurrence of neurodegenerative disease in a subject, comprising:
the step of detecting in a target nucleic acid obtained from the subject the presence or absence of an allelic variant of a KNSL1 gene corresponding to an insertion of a 7 base pair poly-T sequence corresponding to -TTTTTTT- between positions 133354-133355 of SEQ ID NO:484, wherein the presence of said allelic variant is indicative of an increased level of risk for the occurrence of neurodegenerative disease compared to a subject not having said allelic variant. Similar to the polymorphisms in Table 14 herein that have are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484.

Also provided are methods for determining a predisposition for or the occurrence of neurodegenerative disease in a subject, comprising:
the step of detecting in a target nucleic acid obtained from the subject the presence or absence of an allelic variant of a KNSL1 gene corresponding to nucleotide A at position 132370 of SEQ ID NO:484, wherein the presence of said allelic variant is indicative of a predisposition for or the occurrence of neurodegenerative disease.

Also provided are methods for determining a level of risk for the occurrence of neurodegenerative disease in a subject, comprising:
the step of detecting in a target nucleic acid obtained from the subject the presence or absence of an allelic variant of a KNSL1 gene corresponding to nucleotide A at position 132370 of SEQ ID NO:484, wherein the presence of said allelic variant is indicative of an increased level of risk for the occurrence of neurodegenerative disease compared to a subject not having said allelic variant. Similar to the polymorphisms in Table 14 herein that are in linkage disequilibrium with SNP 122260 of SEQ ID NO:484, the polymorphisms that are in linkage disequilibrium with position 132370 of SEQ ID NO-484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484.

Also provided are methods for determining a predisposition for or the occurrence of neurodegenerative disease in a subject, comprising:
the step of detecting in a target nucleic acid obtained from the subject the presence or absence of an allelic variant of polymorphic regions of an IDE gene corresponding to a haplotype comprising particular allelic variants at nucleotide positions complementary to nucleotide positions 122260, 120416, 120288, 80752, and 54795 of SEQ ID NO:484, and wherein the presence of a particular haploptype is indicative of a predisposition for or the occurrence of neurodegenerative disease. In one embodiment, the particular haplotype comprises the nucleotide in IDE at a position complementary to nucleotide 122260 of SEQ ID NO:484 is C, at a position complementary to nucleotide 120416 of SEQ ID NO:484 is T, at a position complementary to nucleotide 120288 of SEQ ID NO:484 is C, at a position complementary to nucleotide 80752 of SEQ ID NO:484 is T, and at position 54795 of SEQ ID NO:484 is C.

Also provided are methods for detecting an altered level of risk for neurodegenerative disease in a subject, comprising:
the step of detecting in a target nucleic acid obtained from the subject the presence of an allelic variant of polymorphic regions of an IDE gene corresponding to a haplotype comprising particular allelic variants at nucleotide positions complementary to nucleotide positions 122260, 120416, 120288, 80752, and 54795 of SEQ ID NO:484, and wherein the presence of a particular haploptype is indicative of an altered level of risk for the neurodegenerative disease compared to a subject not having said allelic variant.

In one embodiment, the particular haplotype comprises the nucleotide in IDE at a position complementary to nucleotide 122260 of SEQ ID NO:484 is C, at a position complementary to nucleotide 120416 of SEQ ID NO:484 is T, at a position complementary to nucleotide 120288 of SEQ ID NO:484 is C, at a position complementary to nucleotide 80752 of SEQ ID NO:484 is T, and at position 54795 of SEQ ID NO:484 is C, and wherein the level of risk is increased compared to a subject that does not have said haplotype. In another embodiment, the particular haplotype comprises the nucleotide in IDE at a position complementary to nucleotide 122260 of SEQ ID NO:484 is T, at a position complementary to nucleotide 120416 of SEQ ID NO:484 is T, at a position complementary to nucleotide 120288 of SEQ ID NO:484 is C, at a position complementary to nucleotide 80752 of SEQ ID NO:484 is T, and at position 54795 of SEQ ID NO:484 is C, and wherein the level of risk is decreased compared to a subject that does not have said haplotype.

In certain embodiments of each of these methods, the neurodegenerative disease is Alzheimer's disease. Likewise, in certain embodiments, each of these methods further comprises detecting the presence or absence of an allelic variant of at least one polymorphic region of at least two different genes associated with neurodegenerative disease, wherein the presence of the allelic variants of two or more genes is indicative of a predisposition for or occurrence of neurodegenerative disease. In one embodiment, one gene of the two different genes is APOE 4. In certain embodiments, the detecting step is selected from the group consisting of sequencing, single-stranded conformation polymorphism, allele specific hybridization, size analysis, primer specific extension, oligonucleotide ligation assay and 5′ nuclease digestion.

Also provided are isolated nucleic acid molecules, comprising at least 14, 16, 18, 20, 22, 24, 26, 28, 30, 35, 40, 45, 50, 60, 70, 80, 90 or at least 100 contiguous nucleotides of a KNSL1 allele derived from SEQ ID NO:348; wherein the contiguous nucleotides include a sequence of 5, 6, 7, 8 or 9 contiguous nucleotides of SEQ ID NO:347 within a group of nucleotide positions corresponding to position 41010 to position 41018, except that between nucleotides at positions 41014 and 41015, a nucleotide sequence corresponding to -AATTT- is inserted.

Also provided are isolated nucleic acid molecules, comprising at least 14, 16, 18, 20, 22, 24, 26, 28, 30, 35, 40, 45, 50, 60, 70, 80, 90 or at least 100 contiguous nucleotides of a KNSL1 allele derived from SEQ ID NO:484; wherein the contiguous nucleotides include a sequence of 5, 6, 7, 8 or 9 contiguous nucleotides of SEQ ID NO:484 position 133351 to position 133359, except that between nucleotides at positions 133354-133355 is either a 6, 7, or 8 base pair poly-T insertion.

Provided herein are polymorphisms of-the IDE, KNSL1, SNCG, TNFRSF6, LIPA and uPA (gene symbol is PLAU) genes and alleles of these genes. In particular embodiments, the polymorphisms are in the human IDE, KNSL1, SNCG, TNFRSF6, LIPA and uPA genes. Polymorphisms of these genes, individually and/or in combination, may be associated with a disease or disorder. Polymorphisms of these genes may be associated with one or more of an IDE-, KNSL1-, SNCG-, TNFRSF6-, LIPA- and/or uPA-mediated disease or disorder. For example, polymorphisms of these genes, individually and/or in combination, may be associated with a disease or disorder involving proteolysis, protein or peptide degradation, and/or interactions between the proteins encoded by these genes and other molecules. Polymorphisms are provided herein that are associated, individually and/or in combination, with a neurodegenerative disease, such as, for example, Alzheimer's disease.

Methods for Determining a Predisposition for or the Occurrence of Neurodegenerative Disease

Methods are provided for determining a predisposition to or occurrence of a neurodegenerative disease in a subject, which include the step of detecting in a target nucleic acid obtained from the subject the presence or absence of an allelic variant of one or more polymorphic regions one or more of a uPA, SNCG, IDE, LIPA, TNFRSF6 or KNSL1 gene, wherein the presence of an allelic variant is indicative of a predisposition for or the occurrence of neurodegenerative disease, such as Alzheimer's disease. The polymorphic region can be a single nucleotide polymorphism (SNP), a single-nucleotide insertion or deletion, a multiple-nucleotide insertion or deletion, a repeat of nucleotides, and the like. A collection of allelic variants at multiple polymorphic regions of one or more genes on a chromosome (haplotype), is often more informative than a single allelic variant in indicating a predisposition to disease. Thus, the present methods for determining a predisposition for or occurrence of neurodegenerative disease, such as Alzheimer's disease, include examination of more than one polymorphic region of a given gene locus. Each allelic variant may be assayed individually or simultaneously using multiplex assay methods.

For each of the methods provided herein, polymorphic regions for the SNCG gene include, but are not limited to, nucleotide positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200 of SEQ ID NO:73, or the complements thereof. In particular embodiments, the nucleotide(s): at position 560 is G or A, at position 590 is A or C, at position 617 is C or T, at position 645 is G or A, at position 915 is T or G, at position 987 is C or A, at position 1723 is A or G, at position 1943 is G or C, at position 1950 is G or A, at position 3151 is A or G, at position 3178 is T or C, at position 3189 is T or C, at position 3284 is G or A, at position 3779 is T or position 3779 is deleted, at position 4156 corresponds to a single nucleotide G that is either inserted or not inserted, at position 4276 is T or A, at position 4311 is C or T, at position 4552 is T or A, at position 4976 is C or position 4976 is deleted, at position 4995 is C or G, at position 5019 is C or T at position 5025 is C or A, at position 5112 is T or A, at position 5136 is T or A, at position 5517 is T or C, at position 2533 is T or G, at position 3371 is A or C, at position 4627 is T or G, at position 4727 is A or G, at position 4813 is A or C, and at position 5200 is G or C.

For each of the methods provided herein, polymorphic regions for the IDE gene include, but are not limited to, nucleotide positions 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565 of SEQ ID NO:187, or the complements thereof. In particular embodiments, the nucleotide(s) in SEQ ID NO:187: at position 2456 is T or G, at position 3279 is T or C, at position 3407 is C or T, at position 42943 is T or C, at position 62498 is T or C, at position 69586 is T or C, at position 107395 is G or A, at position 112114 is G or A, and at position 116662 is T or A.

Additional polymorphic regions for the IDE gene include, but are not limited to, SEQ ID NO:484 nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124, or the complements thereof. In particular embodiments, the complementary nucleotide(s) in SEQ ID NO:484: at position 820 is A or T, at position 7066 is A or G, at position 11758 is T or C, at position 21270 is T or G, at position 22225 is A or T, at position 29294 is C or T, at position 33452 is G or T, at position 33708 is G or A, at position 36982 is C or T, at position 54862 is A or G, at position 77786 is C or A, at position 80594 is G or A, at position 84792 is T or C, at position 84997 is G or T, at position 86682 is C or T, at position 86857 is T or A, at position 88511 is A or G, at position 90437 is G or T, at position 90593 is G or A, at position 91650 is T or C, at position 91870 is G or A, at position 91878 is G or A, at position 92011 is C or T, at position 93618 is T or C, at position 94344 is C or T, at position 94714 is A or G, at position 95671 is A or G, at position 96324 is A or G, at position 97302 is G or A, at position 97370 is G or A, at position 98253 is T or C, at position 98276 is C or T, at position 98385 is A or G, at position 98646 is T or A, at position 98814 is G or A, at position 99597 is C or T, at position 100378 is T or C, at position 101029 is G or A” at position 101265 is C or T, at position 102465 is C or G, at position 103289 is T or G, at position 103967 is C or T at position 105793 is A or G, at position 106076 is G or T, at position 106453 is C or T, at position 106600 is A or G, at position 106995 is G or A, at position 107851 is C or T, at position 108434 is G or C, at position 109096 is C or T, at position 109399 is C or T, at position 109483 is T or G, at position 110870 is G or A, at position 11 1189 is A or G, at position 111972 is G or A, at position 112627 is A or T, at position 112629 is A or T, at position 112631 is T or A, at position 113407 is C or G, at position 114444 is C or G, at position 114482 is G or C, at position 115473 is C or position 115473 is deleted, at position 116681 is G or T, at position 117226 is A or T, at position 117600 is A or G, at position 117802 is C or T, at position 118223 is G or C, at position 120011 is C or T, at position 122260 is A or G, at position 123165 is A or G, at position 123424 is G or A, at position 124352 is A or G, at position 124501 is C or T, at position 124692 is A or G, at position 125113 is T or A, at position 125159 is G or A, at position 126568 is G or C, at position 127166 is C or G, at position 127598 is T or C, at position 127600 is T or C, at position 127609 is T or C, at position 127614 is T or C, at position 127623 is T or C, at position 127662 is G or A, at position 128053 is G or A, at position 128261 is a repeat of -TAAA- occurring 6, 7, or 8 times beginning at position 128261, at position 128289 is A or T, at position 128291 is T or G, at position 128393 is T or G, at position 129444 is C or T.

In a further embodiment, the one or more polymorphic regions of the IDE gene is a haplotype comprising particular allelic variants at nucleotides 2456, 3279, 3407 and 42943 of SEQ ID NO:187. In one embodiment of this haplotype, the nucleotide in IDE at position 2456 of SEQ ID NO:187 is G, at position 3279 of SEQ ID NO:187 is T, at position 3407 of SEQ ID NO:187 is T, and at position 42943 of SEQ ID NO:187 is T. In another embodiment of this haplotype, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is T. In still a further embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is C. In yet another embodiment, the nucleotide in IDE at position 2456 of SEQ ID NO:187 is T, at position 3279 of SEQ ID NO:187 is C, at position 3407 of SEQ ID NO:187 is C, and at position 42943 of SEQ ID NO:187 is C. In another embodiment, the one or more polymorphic regions of the IDE gene is a SNP corresponding to nucleotide 112114 of SEQ ID NO:187, wherein the allelic variant is A.

For each of the methods provided herein, polymorphic regions for the KNSL1 gene include, but are not limited to, nucleotide positions 300, 1152, 14235, 15104, 20815, 35719, 36738-36739, 41015, 42125, 45083, 45887, 56706, 56887, 58524, 62661 and 63802 of SEQ ID NO:348, or the complements thereof. In particular embodiments, the nucleotide(s): at position 300 corresponds to a dinucleotide -CA- that is either inserted or not inserted beginning at position 300, at position 1152 is G or T, at position 14235 corresponds to a single nucleotide T that is either inserted or not inserted, at position 15104 is A or G, at position 20815 is T or C, at position 35719 is T or C, at positions 36738-36739 is a dinucleotide corresponding to CA or AC, at position 41015 corresponds to the oligonucleotide -AATTT- that is either inserted or not inserted beginning at position 41015, at position 42125 is T or G, at position 45083 is C or T, at position 45887 is G or C, at position 56706 is C or T, at position 56887 is A or G, at position 58524 is C or T, at position 62661 is C or T, and at position 63802 is A or C.

Additional polymorphic regions for the KNSL1 gene include, but are not limited to, SEQ ID NO:484 nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153, or the complement thereof. In particular embodiments, the nucleotide(s) in SEQ ID NO:484: at position 130876 is T or C, at position 131378 is G or A, at position 131616 is G or A, at position 131620 is G or A, at position 131688 is T or G, at positions 131998-131203 are CTTTTC- or positions 131998-131203 are deleted, at position 132004 is either a 9, 16, 21, 26, or 29 base pair poly-T repeat beginning at nucleotide 132004, at position 132370 is A or G, at position 132697 is A or G, at position 132968 is C or T, at position 133355 is either a 6, 7 or 8 base pair poly-T repeat beginning at nucleotide 133355, at position 133806 is T or G, at position 134030 is G or A, at position 134291 is A or G, at position 134661 is G or A, at position 137087 is A or G, at position 137142 is G or A, at position 138396 is C or T, at position 140665 is T or G, at position 140736 is A or G, at position 141173 is A or G, at position 142056 is T or C, at position 142777 corresponds to a dinucleotide -AG- that is either inserted or not inserted beginning at position 142777, at position 143025 is G or T, at position 143729 is C or A, at position 144484 is T or A, at position 146181 is T or A, at position 147051 is G or A, at position 147322 is C or T, at position 147707 is G or T, at positions 147842-147845 are -AGTT- or positions 147842-147845 are deleted, at position 148080 is C or T, at position 149026 is either a 17, 18, 19 or 22 base pair -AC-repeat beginning at nucleotide 149026, at position 149044 is either a 22, 24, 28, 30, 32 or 36 base pair -GT- repeat beginning at nucleotide 149044, at position 149389 is A or G, at position 150003 is G or A, at position 150384 is G or T, at position 150454 is C or T, at position 150686 is G or T, at position 151343 is C or T, at position :151961 is C or T, at position 152119 is C or T, at position 153791 is C or G, at position 154328,is A or T, at position 154513 is C or A, at position 154639 is G or A, at position 155049 is T or C, at position 155114 is T or C, at position 158040 is C or A, at position 158895 is G or A, at position 191284 is C or T, at position 192272 is C or T, at position 192698 is A or T, at position 193706 is T or A.

In a further embodiment, the one or more polymorphic regions of the KNSL1 gene is a haplotype comprising particular allelic variants at nucleotides 132370, 133355, 147842 and 178981 of SEQ ID NO:484. In one embodiment, the nucleotide(s) in KNSL1: at position 132370 of SEQ ID NO:484 is A; between positions 133354-133355 of SEQ ID NO:484 is a 6, 7 or 8 base pair poly-T insertion corresponding to -TTTTTT(T)(T)-; at positions 147842-147845 of SEQ ID NO:484 is the 4 base pair insertion corresponding to -AGTT-; and between positions 178980-178981 of SEQ ID NO:484 is the 5 base pair insertion corresponding to -AATTT-. In particular embodiments, the poly-T insertion can be 6 base pairs corresponding to -TTTTTT-; the poly-T insertion can be 7 base pairs corresponding to -TTTTTTT-; or the poly-T insertion can be 8 base pairs corresponding to -TTTTTTTT-. Similar to the polymorphisms in Table 14 herein that are in linkage disequilibrium with SNP 122260 of SEQ ID NO:484, the polymorphisms that are in linkage disequilibrium with position 178980/178981 of SEQ ID NO:484 (a position associated with AD) are also associated with AD, and may therefore be assessed in place of assessing the polymorphism at position 178980/178981 of SEQ ID NO:484 for each of the methods and applications provided herein that assess the polymorphism at position 178980/178981 of SEQ ID NO:484.

For each of the methods provided herein, polymorphic regions for the LIPA gene include, but are not limited to, nucleotide positions 1197, 1307-1309, 1841, 1852, 2075, 6063, 6173, 6194, 7820, 25283, 28453-28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969 of SEQ ID NO:468, or the complements thereof. In particular embodiments, the nucleotide(s): at position 1197 is C or G, the nucleotides at positions 1307-1309 are ATC or positions 1307-1309 are deleted, the nucleotide at position 1841 is A or C, at position 1852 is G or A, at position 2075 is G or A, at position 6063 is G or T, at position 6173 is A or C, at position 6194 is G or A, at position 7820 is C or G, at position 25283 is G or C, the nucleotides at positions 28453-28465 are -TCCGCGAGAGGGC- or positions 28453-28465 are deleted, the nucleotide at position 28543 is C or T, at position 28746 is A or C, at position 29904 is G or A, at position 37861 is C or T, at position 39834 is T or A, and at position 40018 is C or T.

In a further embodiment, the one or more polymorphic regions of the LIPA gene is a haplotype comprising particular allelic variants at nucleotides 1852, 6063 and 7820 of SEQ ID NO:468. In this embodiment, the nucleotide in LIPA at position 1852 of SEQ ID NO:468 is A, at position 6063 of SEQ ID NO:468 is G, and at position 7820 of SEQ ID NO:468 is C.

For each of the methods provided herein, polymorphic regions for the TNFRSF6 gene include, but are not limited to, nucleotide positions 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026 of SEQ ID NO:403, or the complements thereof. In particular embodiments, the nucleotide(s): at position 1530 is T or C, at position 1550 is A or G, at position 14525 is G or A, at position 14714 is C or T, at position 18982 is G or C, at position 19069 is A or G, at position 20412 is A or G, at position 20552 is A or G, at position 23199 is G or A, at position 23416 is T or C, at position 24890 is A or G, at position 26359 is A or T, at position 1926 is G or A, at position 2269 is G or A, at position 18934 is C or T, at position 19227 is C or T, and at position 22026 is C or G.

For each of the methods provided herein, polymorphic regions for the uPA (PLAU) gene include, but are not limited to nucleotide positions selected from the group of uPA nucleotide positions of SEQ ID NO:559 or 560 consisting of 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848, 7908, and the complementary positions thereof; and positions of SEQ ID NO:563 consisting of 79, 93, 256, 385 and 714, and the complementary positions thereof. In particular embodiments, the nucleotide(s) in SEQ ID NO:559 or 560: at position 9 is A or C, at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, and at position 6532 is T or C, and the complements thereof; and the nucleotide in SEQ ID NO:563: at position 79 is T or C, at position 93 is a C or position 93 is deleted, at position 256 is G or T, at position 385 is C or T, at position 714-715 is the dinulceotide -GT- or the -GT- dinucleotide is deleted.

In particular embodiments of the methods provided herein the one or more uPA polymorphisms occur at nucleotide positions corresponding to nucleotide positions selected from the group of nucleotide positions of SEQ ID NO:569 or 560 consisting of 401, 515, 748 and 1752 and the complementary positions thereof; and of SEQ ID NO:563 consisting of 93 and 714-715, and the complementary positions thereof. In other embodiments, the one or more uPA polymorphisms occur at nucleotide positions corresponding to nucleotide positions selected from the group of nucleotide positions of SEQ ID NO:559 or 560 consisting of 9, 401, 464, 515, 748, 1229, 1356, 1752, 1942, 2127, 2543, 3029, 3169, 3799, 3947, 4808, 5287, and 6532 and the complementary positions thereof; and positions of SEQ ID NO:563 consisting of 79, 93, 256, 385 and 714, and the complementary positions thereof. In yet other embodiments, the one or more uPA polymorphisms occur at nucleotide positions corresponding to nucleotide positions selected from the group of nucleotide positions of SEQ ID NO:559 or 560 52 consisting of 401, 464, 515, 748, 1229, 1356, 1752, 1942, 2127, 2543, 3029 and 5287 and the complementary positions thereof; and positions of SEQ ID NO:563 consisting of 79, 93, 256, 385 and 714, and the complementary positions thereof. In another embodiment, the one or more uPA polymorphisms occur at nucleotide positions corresponding to nucleotide positions selected from the group of nucleotide positions of SEQ ID NO:559 or 560 consisting of 9, 178, 401, 464, 515, 748; and positions of SEQ ID NO:563 consisting of 79, 93, 256, 385 and 714; and the complementary positions thereof. In yet other embodiments, the one or more uPA polymorphisms occur at nucleotide positions corresponding to nucleotide positions selected from the group of nucleotide positions of SEQ ID NO:559 or 560 consisting of 401, 515 and 748; and positions of SEQ ID NO:563 consisting of 93 and 714; and the complementary positions thereof. In another embodiment, the uPA polymorphisms occur at nucleotide positions corresponding to nucleotide positions 3169, 3947 and 6532 of SEQ ID NO:559 or 560 and the complementary positions thereof.

Further provided are methods for determining a predisposition for or the occurrence of a neurodegenerative disease, such as Alzheimer's disease, comprising detecting the presence or absence of an allelic variant of at least one polymorphic region of at least two different genes associated with neurodegenerative disease, wherein the presence of the allelic variants of two or more genes is indicative of a predisposition for or occurrence of neurodegenerative disease. In a′ particular embodiment, the method involves detecting the presence or absence of an allelic variant of at least one polymorphic region of one or more of a uPA, SNCG, IDE, LIPA, TNFRSF6 or KNSL1 gene, and at least one polymorphic region of a gene associated with Alzheimer's disease that is different from uPA, SNCG, IDE, LIPA, TNFRSF6 and KNSL1, wherein the presence of the two or more allelic variants is indicative of a predisposition for or the occurrence of Alzheimer's disease. Other or different genes associated with Alzheimer's disease include, but are not limited to, APOE4, and the like.

The detection of the presence or absence of an allelic variant includes, but is not limited to, methods such as sequencing, allele specific hybridization, primer specific extension, oligonucleotide ligation assay, restriction enzyme site analysis, size analysis, 5′ nuclease digestion and single-stranded conformation polymorphism analysis.

Nucleic Acid Molecules

Further provided are isolated nucleic acid molecules encoding the novel polymorphisms of uPA, SNCG, IDE, LIPA, TNFRSF6 and KNSL1 genes. The isolated nucleic acid molecules include the following.

An isolated nucleic acid molecule, comprising at least 14, 16, 18, 20, 22, 24, 26, 28, 30, 35, 40, 45, 50, 60, 70, 80, 90 or 100 contiguous of a SNCG allele; wherein the contiguous nucleotides include a sequence of 5, 6, 7, 8 or 9 contiguous nucleotides of SEQ ID NO:72 selected from the group of nucleotide positions corresponding to one or more of: position 613 to position 621, except that the nucleotide at position 617 is replaced with a nucleotide selected from the group consisting of G, T and A; position 641 to position 649, except that the nucleotide at position 645 is replaced with a nucleotide selected from the group consisting of C, T and A; position 911 to position 919, except that the nucleotide at position 915 is replaced with a nucleotide selected from the group consisting of G, C and A; position 983 to position 991, except that the nucleotide at position 987 is replaced with a nucleotide selected from the group consisting of G, T and A; position 1946 to position 1954, except that the nucleotide at position 1950 is replaced with a nucleotide selected from the group consisting of C, T and A; position 3147 to position 3155, except that the nucleotide at position 3151 is replaced with a nucleotide selected from the group consisting of G, C and T; position 3174 to position 3182, except that the nucleotide at position 3178 is replaced with a nucleotide selected from the group consisting of G, C and A; position 3185 to position 3193, except that the nucleotide at position 3189 is replaced with a nucleotide selected from the group consisting of G, C and A; position 3280 to position 3288, except that the nucleotide at position 3284 is replaced with a nucleotide selected from the group consisting of T, C and A; position 3775 to position 3783, except that the nucleotide at position 3779 is replaced with a nucleotide selected from the group consisting of G, C and A; position 4152 to position 4160, except that between nucleotides at positions 4155 and 4156 a G is inserted; position 4272 to position 4280, except that the nucleotide at position 4276 is replaced with a nucleotide selected from the group consisting of G, C and A; position 4307 to position 4315, except that the nucleotide at position 4311 is replaced with a nucleotide selected from the group consisting of G, T and A; position 4548 to position 4556, except that the nucleotide at position 4552 is replaced with a nucleotide selected from the group consisting of G, C and A; position 4972 to position 4980, except that the C nucleotide at position 4976 is deleted; position 4991 to position 4999, except that the nucleotide at position 4995 is replaced with a nucleotide selected from the group consisting of G, T and A; position 5021 to position 5029, except that the nucleotide at position 5025 is replaced with a nucleotide selected from the group consisting of G, T and A; position 5132 to position 3140, except that the nucleotide at position 5136 is replaced with a nucleotide selected from the group consisting of G, C and A; position 5513 to 5521, except that the nucleotide at position 5517 is replaced with a nucleotide from the group consisting of G, C and A; position 2529 to position 2537, except that the nucleotide at position 2533 is replaced with a nucleotide selected from the group consisting of G, C and A; position 3367 to position 3375, except that the nucleotide at position 3371 is replaced with a nucleotide selected from the group consisting of G, C and T; position 4623 to position 4631, except that the nucleotide at position 4627 is replaced with a nucleotide selected from the group consisting of G, C and A; position 4723 to position 4731, except that the nucleotide at position 4727 is replaced with a nucleotide selected from the group consisting of G, T and C; position 4809 to position 4817, except that the nucleotide at position 4813 is replaced with a nucleotide selected from the group consisting of G, C and T; and position 5196 to position 5204, except that the nucleotide at position 5200 is replaced with a nucleotide from the group consisting of T, C and A.

An isolated nucleic acid molecule, comprising at least 14, 16, 18, 20, 22, 24, 26, 28, 30, 35, 40, 45, 50, 60, 70, 80, 90 or 100 contiguous of an IDE allele; wherein the contiguous nucleotides comprise a sequence of 5, 6, 7, 8 or 9 contiguous nucleotides of SEQ ID NO:186, or the complement thereof, selected from the group of nucleotide ranges consisting of one/or more of: position 2452 to position 2460, except that the nucleotide at position 2456 is replaced with a nucleotide selected from the group consisting of C, G and A; position 3275 to position 3283, except that the nucleotide at position 3279 is replaced with a nucleotide selected from the group consisting of C, G and A; position 3403 to position 3411, except that the nucleotide at position 3407 is replaced with a nucleotide selected from the group consisting of T, G and A; position 42939 to position 42947, except that the nucleotide at position 42943 is replaced with a nucleotide selected from the group consisting of C, G and A; position 62494 to position 62502, except that the nucleotide at position 62498 is replaced with a nucleotide selected from the group consisting of C, G and A; position 69582 to position 69590, except that the nucleotide at position 69586 is replaced with a nucleotide selected from the group consisting of G, C and A; position 107391 to position 107399, except that the nucleotide at position 107395 is replaced with a nucleotide selected from the group consisting of T, C and A; and position 112110 to position 112118, except that the nucleotide at position 112114 is replaced with a nucleotide selected from the group consisting of C, T and A; and/or a sequence of 5, 6, 7, 8 or 9 contiguous nucleotides complementary to SEQ ID NO:484 selected from the group of nucleotide ranges consisting of one or more of:
position 816 to position 824, except that the complementary nucleotide at position 820 is replaced with a nucleotide selected from the group consisting of C, G and T;
position 7062 to position 7070, except that the complementary nucleotide at position 7066 is replaced with a nucleotide selected from the group consisting of T, C and G;
position 21266 to position 21274, except that the complementary nucleotide at position 21270 is replaced with a nucleotide selected from the group consisting of A, C and G.;
position 22221 to position 22229, except that the complementary nucleotide at position 22225 is replaced with a nucleotide selected from the group consisting of C, G and T;
position 29290 to position 29298, except that the complementary nucleotide at position 29294 is replaced with a nucleotide selected from the group consisting of A, G and T;
position 33448 to position 33456, except that the complementary nucleotide at position 33452 is replaced with a nucleotide selected from the group consisting of A, C and G;
position 33703 to position 33712, except that the complementary nucleotide at position 33708 is replaced with a nucleotide selected from the group consisting of A, C and T;
position 36978 to position 36986, except that the complementary nucleotide at position 36982 is replaced with a nucleotide selected from the group consisting of A, G, and T;
position 77782 to position 77790, except that the complementary nucleotide at position 77786 is replaced with a nucleotide selected from the group consisting of A, G and T;
position 80590 to position 80598, except that the complementary nucleotide at position 80594 is replaced with a nucleotide selected from the group consisting of A, C and T;
position 84993 to position 85001, except that the complementary nucleotide at position 84997 is replaced with a nucleotide selected from the group consisting of A, C, and G;
position 86678 to position 86686, except that the complementary nucleotide at position 86682 is replaced with a nucleotide selected from the group consisting of A, G and T;
position 86853 to position 86861, except that the complementary nucleotide at position 86857 is replaced with a nucleotide selected from the group consisting of A, C and G;
position 88507 to position 88515, except that the complementary nucleotide at position 88511 is replaced with a nucleotide selected from the group consisting of C, G and T;
position 90433 to position 90441, except that the complementary nucleotide at position 90437 is replaced with a nucleotide selected from the group consisting of A, C and T;
position 90581 to position 90597, except that the complementary nucleotide at position 90593 is replaced with a nucleotide selected from the group consisting of A, C and T;
position 91546 to position 91654, except that the complementary nucleotide at position 91650 is replaced with a nucleotide selected from the group consisting of A, C and G;
position 91864 to position 91874, except that the complementary nucleotide at position 91870 is replaced with a nucleotide selected from the group consisting of C, G and T;
position 91874 to position 91882, except that the complementary nucleotide at position 91878 is replaced with a nucleotide selected from the group consisting of A, C and T;
position 92007 to position 92015, except that the complementary nucleotide at position 92011 is replaced with a nucleotide selected from the group consisting of A, G and T;
position 93614 to position 93622, except that the complementary nucleotide at position 93618 is replaced with a nucleotide selected from the group consisting of A, C and G;
position 94340 to position 94348, except that the complementary nucleotide at position 94344 is replaced with a nucleotide selected from the group consisting of A, C and G;
position 94710 to position 94718, except that the complementary nucleotide at position 94714 is replaced with a nucleotide selected from the group consisting of C, G and T;
position 95667 to position 95675, except that the complementary nucleotide at position 95671 is replaced with a nucleotide selected from the group consisting of A, C and G;
position 96320 to position 96328, except that the complementary nucleotide at position 96324 is replaced with a nucleotide selected from the group consisting of A, G and T;
position 97298 to position 97306, except that the complementary nucleotide at position 97302 is replaced with a nucleotide selected from the group consisting of A, C and T;
position 37366 to position 97374, except that the complementary nucleotide at position 97370 is replaced with a nucleotide selected from the group consisting of A, C and T;
position 98249 to position 98257, except that-the complementary nucleotide at position 98253 is replaced with a nucleotide selected from the group consisting of A, C and G;
position 98381 to position 98389, except that the complementary nucleotide at position 98385 is replaced with a nucleotide selected from the group consisting of C, G and T;
position 98641 to position 98650, except that the complementary nucleotide at position 98646 is replaced with a nucleotide selected from the group consisting of C, G and T;
position 98810 to position 98818, except that the complementary nucleotide at position 98814 is replaced with a nucleotide selected from the group consisting of A, C and T;
position 99593 to position 99601, except that the complementary nucleotide at position 99597 is replaced with a nucleotide selected from the group consisting of A, C and G;
position 100374 to position 100382, except that the complementary nucleotide at position 100378 is replaced with a nucleotide selected from the group consisting of A, C and G;
position 101025 to position 101033, except that the complementary nucleotide at position 101029 is replaced with a nucleotide selected from the group consisting of C, G and T;
position 101261 to position 101269, except that the complementary nucleotide at position 1Q1265 is replaced with a nucleotide selected from the group consisting of A, C and G;
position 102461 to position 102469, except that the complementary nucleotide at position 102465 is replaced with a nucleotide selected from the group consisting of A, G and T;
position 103285 to position 103293, except that the complementary nucleotide at position, 103289 is replaced with a nucleotide selected from the group consisting of A, C and T;
position 103963 to position 103.971, except that the complementary nucleotide at position 103967 is replaced with a nucleotide selected from the group consisting of A, G and T;
position 105789 to position 105797, except that the complementary nucleotide at position 105793 is replaced with a nucleotide selected from the group consisting of C, G and T;
position 106072 to position 106080, except that the complementary nucleotide at position 106076 is replaced with a nucleotide selected from the group consisting of A, C and T;
position 106991 to position 106999, except that the complementary nucleotide at position 106995 is replaced with a nucleotide selected from the group consisting of A, C and T;
position 107847 to position 107855, except that the complementary nucleotide at position 107851 is replaced with a nucleotide selected from the group consisting of A, G and T;
position 108430 to position 108438, except that the complementary nucleotide at position 108434 is replaced with a nucleotide selected from the group consisting of A, C and T;
position 109092 to position 109100, except that the complementary nucleotide at position 109096 is replaced with a nucleotide selected from the group consisting of A, G and T;
position 109395 to position 1093403, except that the complementary nucleotide at position 109399 is replaced with a nucleotide selected from the group consisting of A, G and T;
position 109479 to position 109487, except that the complementary nucleotide at position 109483 is replaced with a nucleotide selected from the group consisting of A, C And T;
position 110866 to position 110874, except that the complementary nucleotide at position 110870 is replaced with a nucleotide selected from the group consisting of A, C and T;
position 111185 to position 111193, except that the complementary nucleotide at position 111189 is replaced with a nucleotide selected from the group consisting of C, G and T;
position 111968 to position 111976, except that the complementary nucleotide at position 111972 is replaced with a nucleotide selected from the group consisting of A, C and T;
position 112623 to position 112631, except that the complementary nucleotide at position 112627 is replaced with a nucleotide selected from the group consisting of C! G and T;
position 113403 to position 113411, except that the complementary nucleotide at position 113407 is replaced with a nucleotide selected from the group consisting of A, C and T;
position 114478 to position 114486, except that the complementary nucleotide at position 114482 is replaced with a nucleotide selected from the group consisting of A, C and T;
position 115469 to position 115477, except that the complementary nucleotide at position 115473 is deleted;
position 116677 to position 116685, except that the complementary nucleotide at position 116681 is replaced with a -nucleotide selected from the group consisting of A, C and G;
position 117222 to position 117230, except that the complementary nucleotide at position 117226 is replaced with a nucleotide selected from the group consisting of C, G and T;
position 117596 to position 117604, except that the complementary nucleotide at position 117600 is replaced with a nucleotide selected from the group consisting of T, C and G;
position 118219 to position 118227, except that the complementary nucleotide at position 118223 is replaced with a nucleotide selected from the group consisting of T, A and G;
position 120007 to position 120015, except that the complementary nucleotide at position 120011 is replaced with a nucleotide selected from the group consisting of A, G and T;
position 122256 to position 122264, except that the complementary nucleotide at position 122260 is replaced with a nucleotide selected from the group consisting of A, C and G;
position 123161 to position 123169, except that the complementary nucleotide at position 123165 is replaced with a nucleotide selected from the group consisting of A, C and G;
position 123420 to position 123428, except that the complementary nucleotide at position 123424 is replaced with a nucleotide selected from the group consisting of A, G and T;
position 124348 to position 124356, except that the complementary nucleotide at position 124352 is replaced with a nucleotide selected from the group consisting of A, C and T;
position 124497 to position 124505, except that the complementary nucleotide at position 124501 is replaced with a nucleotide selected from the group consisting of A, C and T;
position 124688 to position 124696, except that the complementary nucleotide at position 124692 is replaced with a nucleotide selected from the group consisting of A, C and G;
position 125109 to position 125117, except that the complementary nucleotide at position 125113 is replaced with a nucleotide selected from the group consisting of C, G and T;
position 125154 to position 125163, except that the complementary nucleotide at position 125159 is replaced with a nucleotide selected from the group consisting of A, G and T;
position 126564 to position 126572, except that the complementary nucleotide at position 126568 is replaced with a nucleotide selected from the group consisting of A, G and T;
position 127162 to position 127170, except that the complementary nucleotide at position 127166 is replaced with a nucleotide selected from the group consisting of A, C and T;
position 127594 to position 127602, except that the complementary nucleotide at position 127598 is replaced with a nucleotide selected from the group consisting of C, G and T;
position 127596 to position 127604, except that the complementary nucleotide at position 127600 is replaced with a nucleotide selected from the group consisting of C, G and T;
position 127605 to position 127613, except that the complementary nucleotide at position 127609 is replaced with a nucleotide selected from the group consisting of C, G and T;
position 127610 to position 127618, except that the complementary nucleotide at position 127614 is replaced with a nucleotide selected from the group consisting of C, G and T;
position 127619 to position 127627, except that the complementary nucleotide at position 127623 is replaced with a nucleotide selected from the group consisting of C, G and T;
position 127658 to position 127666, except that the complementary nucleotide at position 127662 is replaced with a nucleotide selected from the group consisting of A, C and G;
position 128049 to position 128057, except that the complementary nucleotide at position 128053 is replaced with a nucleotide selected from the group consisting of A, C and T;
position 128257 to position 128296, except that at complementary nucleotide positions 128261-128292, 1 or 2-TAAA- repeats are deleted;
position 128285 to position 128293, except that the complementary nucleotide at position 128289 is replaced with a nucleotide selected from the group consisting of A, C and G;
position 128287 to position 128295, except that the complementary nucleotide at position 128291 is replaced with a nucleotide selected from the group consisting of C, G and T;
position 128389 to position 128397, except that the complementary nucleotide at position 128393 is replaced with a nucleotide selected from the group consisting of C, G and T; and
position 129440 to position 129448, except that the complementary nucleotide at position 129444 is replaced with a nucleotide selected from the group consisting of C, G and T.

An isolated nucleic acid molecule, comprising at least 14, 16, 18, 20, 22, 24, 26, 28, 30, 35, 40, 45, 50, 60, 70, 80, 90 or 100 contiguous of a KNSL1 allele; wherein the contiguous nucleotides include a sequence of 5, 6, 7, 8 or 9 contiguous nucleotides of SEQ ID NO:347 selected from the group of nucleotide positions corresponding to one or more of: position 295 to position 303, except that between nucleotides 299 and 300 a dinucleotide corresponding to -CA- is inserted; position 1148 to position 1156, except that the nucleotide at position 1152 is replaced with a nucleotide selected from the group consisting of A, C and T; position 14230 to position 14238, except that between nucleotides 14234 and 14235, a nucleotide selected from the group consisting of T, C, G and A is inserted; position 15100 to position 15108, except that the nucleotide at position 15104 is replaced with a nucleotide selected from the group consisting of T, C and G; position 20811 to position 20819, except that the nucleotide at position 20815 is replaced with a nucleotide selected from the group consisting of C, G and A; position 36734 to position 36742, except that the nucleotides at positions 36738 and 36739 are replaced with nucleotides corresponding to -AC-, respectively; position 41010 to position 41018, except that between nucleotides at positions 41014 and 41015, a nucleotide sequence corresponding to -AATTT- is inserted; position 42121 to position 42129, except that the nucleotide at position 42125 is replaced with a nucleotide selected from the group consisting of C, G and A; position 56702 to position 56710, except that the nucleotide at position 56706 is replaced with a nucleotide selected from the group consisting of G, T and A; position 56883 to position 56891, except that the nucleotide at position 56887 is replaced with a nucleotide selected from the group consisting of C, T and G; position 58520 to position 58528, except that the nucleotide at position 58524 is replaced with a nucleotide selected from the group consisting of G, T and A; and/or a sequence of 5, 6, 7, 8 or 9 contiguous nucleotides of SEQ ID NO:484 selected from the group of nucleotide ranges consisting of one or more of:
position 130872 to position 130880, except that the nucleotide at position 130876 is replaced with a nucleotide selected from the group consisting of A, G and T;
position 131374 to position 131382, except that the nucleotide at position 131378 is replaced with a nucleotide 4 selected from the group consisting of C, G and T;
position 131612 to position 131620, except that the nucleotide at position 131616 is replaced with a nucleotide selected from the group consisting of C, G and T;
position 131616 to position 131624, except that the nucleotide at position 131620 is replaced with a nucleotide selected from the group consisting of C, G and T;
position 131684 to position 131692, except that the nucleotide at position 131688 is replaced with a nucleotide selected from the group consisting of A, C and G;
position 131994 to position 132007, except that the nucleotides at positions 131998-132003 are deleted;
position 132000 to position 132036, except that the 29 base pair poly-T repeat at nucleotide positions 132004-132032 is replaced with either a 9, 16, 21 or 26 base pair poly-T repeat;
position 132693 to position 132701, except that the nucleotide at position 132697 is replaced with a nucleotide selected from the group consisting of C, G and T;
position 132964 to position 132972, except that the nucleotide at position 132968 is replaced with a nucleotide selected from the group consisting of A, G and T;
position 133351 to position 133359, except that between nucleotides at positions 133354-133355 is either a 6, 7, or 8 base pair poly-T insertion;
position 133802 to position 133810, except that the nucleotide at position 133806 is replaced with a nucleotide selected from the group consisting of A, C and G;
position 134026 to position 134034, except that the nucleotide at position 134030 is replaced with a nucleotide selected from the group consisting of A, C and T;
position 134287 to position 134295, except that the nucleotide at position 134291 is replaced with a nucleotide selected from the group consisting of A, C and T;
position 134657 to position 134665, except that the nucleotide at position 134661 is replaced with a nucleotide selected from the group consisting of A, C and T;
position 137083 to position 137091, except that the nucleotide at position 137087 is replaced with a nucleotide selected from the group consisting of C, G and T;
position 137138 to position 137146, except that the nucleotide at position 137142 is replaced with a nucleotide selected from the group consisting of A, C and T;
position 138392 to position 138370, except that the nucleotide at position 138396 is replaced with a nucleotide selected from the group consisting of A, G and T;
position 140661 to position 140669, except that the nucleotide at position 140665 is replaced with a nucleotide selected from the group consisting of A, C and G;
position 140732 to position 140740, except that the nucleotide at position 140736 is replaced with a nucleotide selected from the group consisting of C, G and T;
position 141169 to position 141177, except that the nucleotide at position 141173 is replaced with a nucleotide selected from the group consisting of C, G and T;
position 142052 to position 142070, except that the nucleotide at position 142056 is replaced with a nucleotide selected from the group consisting of A, C and G;
position 142773 to position 142781, except that between nucleotides at positions 142776-142777 is an -AG- insertion;
position 143021 to position 143029, except that the nucleotide at position 143025 is replaced with a nucleotide selected from the group consisting of A, C and G;
position 143725 to position 143733, except that the nucleotide at position 143729 is replaced with a nucleotide selected from the group consisting of A, G and T;
position 144480 to position 144488, except that the nucleotide at position 144484 is replaced with a nucleotide selected from the group consisting of C, G and T;
position 146177 to position 146185, except that the nucleotide at position 146181 is replaced with a nucleotide selected from the group consisting of A, C and G;
position 147047 to position 147055, except that the nucleotide at position 147051 is replaced with a nucleotide selected from the group consisting of A, C and T;
position 147318 to position 147326, except that the nucleotide at position 147322 is replaced with a nucleotide selected from the group consisting of A, G and T;
position 147703 to position 147711, except that the nucleotide at position 147707 is replaced with a nucleotide selected from the group consisting of A, C and T;
position 147838 to position 147849, except that the nucleotides at positions 147842-147845 are deleted;
position 148076 to position 148084, except that the nucleotide at position 148080 is replaced with a nucleotide selected from the group consisting of A, G and T;
position 149022 to position 149047, except that the 18 nucleotide -AC- repeat at positions 149026-149043 is replaced with either a 17, 19 or 22 base pair -AC- repeat;
position 149040 to position 149048, except that the 30 nucleotide -GT- repeat at positions 149044-149073 is replaced with either a 22, 24, 28 or 32 base pair -GT- repeat;
position 149385 to position 149393, except that the nucleotide at position 149389 is replaced with a nucleotide selected from the group consisting of A, C and T;
position 149999 to position 150007, except that the nucleotide at position 150003 is replaced with a nucleotide, selected from the group consisting of A, C and T;
position 150380 to position 150388, except that the nucleotide at position 150384 is replaced with a nucleotide selected from the group consisting of A, C and T;
position 150450 to position 150458, except that the nucleotide at position. 150454 is replaced with a nucleotide selected from the group consisting of A, G and T;
position 150682 to position 150690, except that the nucleotide at position 150686 is replaced with a nucleotide selected from the group consisting of A, C and T;
position 151339 to position 151347, except that the nucleotide at position 151343 is replaced with a nucleotide selected from the group consisting of A, G and T;
position 151957 to position 151965, except that the nucleotide at position 151961 is replaced with a nucleotide selected from the group consisting of A, G and T;
position 152115 to position 152123, except that the nucleotide at position 152119 is replaced with a nucleotide selected from the group consisting of A, G and T;
position 153787 to position 153795, except that the nucleotide at position 153791 is replaced with a nucleotide selected from the group consisting of A, G and T;
position 154324 to position 154332, except that the nucleotide at position 154328 is replaced with a nucleotide selected from the group consisting of C, G and T;
position 154509 to position 154517, except that the nucleotide at position 154513 is replaced with a nucleotide selected from the group consisting of A, G and T;
position 154635 to position 154643, except that the nucleotide at position 154639 is replaced with a nucleotide selected from the group consisting of A, C and T;
position 155045 to position 155053, except that the nucleotide at position 155049 is replaced with a nucleotide selected from the group consisting of A, C and G;
position 155110 to position 155118, except that the nucleotide at position 155114 is replaced with a nucleotide selected from the group consisting of A, C and G;
position 158036 to position 158044, except that the nucleotide at position 158040 is replaced with a nucleotide selected from the group consisting of A, G and T.;
position 158891 to position 158899, except that the nucleotide at position 158895 is replaced with a nucleotide selected from the group consisting of A, C and T;
position 191280 to position 191288, except that the nucleotide at position 191284 is replaced with a nucleotide selected from the group consisting of A, G and T;
position 192268 to position 192276, except that the nucleotide at position 192272 is replaced with a nucleotide selected from the group consisting of A, G and T;
position 192694 to position 192702, except that the nucleotide at position 192698 is replaced with a nucleotide selected from the group consisting of C, G and T; and
position 193702 to position 193710, except that the nucleotide at position 193706 is replaced with a nucleotide selected from the group consisting of A, C and G.

An isolated nucleic acid molecule, comprising at least 14, 16, 18, 20, 22, 24, 26, 28, 30, 35, 40, 45, 50, 60, 70, 80, 90 or 100 contiguous of a LIPA allele; wherein the contiguous nucleotides include a sequence of 5, 6, 7, 8 or 9 contiguous nucleotides of SEQ ID NO:467 selected from the group of nucleotide positions corresponding to one or more of: position 1193 to position 1201, except that the nucleotide at position 1197 is replaced with a nucleotide selected from the group consisting of T, G and A; position 1303 to position 1313, except that the nucleotides at positions 1307-1309 are deleted; position 6059 to position 6067, except that the nucleotide at position 6063 is replaced with a nucleotide selected from the group consisting of T, C and A; position 7816 to position 7824, except that the nucleotide at position 7820 is replaced with a nucleotide selected from the group consisting of T, G and A; position 28449 to position 28469, except that the nucleotides at positions 28453-28465 are deleted; position 28539 to position 28547, except that the nucleotide at position 28543 is replaced with a nucleotide selected from the group consisting of G, T and A; position 28742 to position 28750, except that the nucleotide at position 28746 is replaced with a nucleotide selected from the group consisting of C, T and G. In a particular embodiment, at least 16 contiguous nucleotides of a LIPA allele are contained in SEQ ID NO:467.

An isolated nucleic acid molecule, comprising at least 14, 16, 18, 20, 22, 24, 26, 28, 30, 35, 40,.45, 50, 60, 70, 80, 90 or 100 contiguous of a TNFRSF6 allele; wherein the contiguous nucleotides include a sequence of 5, 6, 7, 8 or 9 contiguous nucleotides of SEQ ID NO:402 selected from the group of nucleotide positions corresponding to one or more of: position 1526 to position 1534, except that the nucleotide at position 1530 is replaced with a nucleotide selected from the group consisting of C, G and A; position 14521 to position 14529, except that the nucleotide at position 14525 is replaced with a nucleotide selected from the group consisting of C, T and-A; position 14710 to position 14718, except that the nucleotide at position 14714 is replaced with a nucleotide selected from the group consisting of T, G and A; position 19065 to position 19073, except that the nucleotide at position 19069 is replaced with a nucleotide selected from the group consisting of G, C and T; position 20408 to position 20416, except that the nucleotide at position 20412 is replaced with a nucleotide selected from the group consisting of C, T and G; position 20548 to position 20556, except that the nucleotide at position 20552 is replaced with a nucleotide selected from the group consisting of T, G and C; position 23195 to position 23203, except that the nucleotide at position 23199 is replaced with a nucleotide selected from the group consisting of C, T and A; position 23412 to position 23420, except that the nucleotide at position 23416 is replaced with a nucleotide selected from the group consisting of C, A and G, position 1922 to position 1930, except that the nucleotide at position 1926 is replaced with a nucleotide selected from the group consisting of C, T and A; and position 2265 to position 2273, except that the nucleotide at position 2269 is replaced with a nucleotide selected from the group consisting of C, A and T. In a particular embodiment the at least 14, 16, 18, 20, 22, 24, 26, 28 and 30 contiguous of a TNFRSF6 allele are contained in SEQ ID NO:402.

An isolated nucleic acid molecule, comprising at least 14, 16, 18, 20, 22, 24, 26, 28 or at least 30 contiguous nucleotides of a uPA gene; wherein the contiguous nucleotides include a sequence of 5, 6, 7, 8 or 9 contiguous nucleotides of SEQ ID NO: 559 or 560, or the complement thereof, selected from the group of nucleotide sequences of SEQ ID NO: 559 or 560 consisting of:
position 397 to position 405, wherein the nucleotide at position 401 is selected from the group consisting of A, T and C; position 511 to position 519, wherein the nucleotide at position 515 is selected from the group consisting of T, G and A; position 744 to position 752, wherein the nucleotide at position 748 is selected from the group consisting of T, C and A; and position 1748 to position 1756, wherein the nucleotide at position 1752 is selected from the group consisting of C, G and A; or of SEQ ID NO: 563 consisting of:
position 89 to position 97, wherein the C nucleotide at position 93 is deleted; and position 710 to position 719, wherein the nucleotides at positions 714-715 are deleted.

Also provided are isolated nucleic acid molecules of the above-described alleles comprising at least 20, 30, 40, 50, 60, 70, 80, 90 or 100 contiguous nucleotides of a uPA, SNCG, IDE, LIPA, TNFRSF6 or KNSL1 allele.

Further provided are nucleic acid vectors comprising the nucleic acid molecules, including cDNAs, described herein and cells containing these nucleic acid vectors.

Primers, Probes and Antisense Nucleic Acid

Further provided are primers, probes and antisense nucleic acid molecules capable of specifically hybridizing to uPA, SNCG, IDE, LIPA, TNFRSF6 and KNSL1 genes or cDNA under conditions of moderate or high stringency. Also provided are primer pairs capable of specifically amplifying all, or a portion of, any of the nucleic acid molecules disclosed herein. The primers, probes or antisense molecules can also comprise at least 14, 16, 18, 20, 22, 24, 26, 28, 30, 35, 40, 45, 50, 60, 70, 80, 90 or 100 contiguous nucleotides of a uPA, SNCG, IDE, LIPA, TNFRSF6 or KNSL1 allele, or the complements thereof.

Exemplary primers, probes or antisense nucleic acid molecules comprise a sequence of nucleotides that specifically hybridizes adjacent to, or at a polymorphic region of: a SNCG allele spanning a nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517; an IDE allele spanning a nucleotide position of SEQ ID NO:187, or the complement thereof, selected from the group consisting of nucleotide positions 2456, 69586, 107395, 112114, and 116662; or and IDE allele spanning a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444; a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a KNSL1 allele spanning a nucleotide position of SEQ ID NO:484, or the complement thereof, selected from the group consisting of nucleotide positions 130876, 131378, 131616, 131620, 131688, 131998, 132004, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706; a LIPA allele spanning a nucleotide position of SEQ ID NO:468, or the complement thereof, selected from the group consisting of nucleotide positions 1197, 7820, 28543 and 28746; and/or a TNFRSF6 allele spanning a nucleotide position of SEQ ID NO:403, or the complement thereof, selected from the group consisting of nucleotide positions 1530, 14525, 14714, 19069, 20412, 20552, 23199, 23416, 1926 and 2269.

Methods to Predict Drug Response

The presence or absence of one or more allelic variants of uPA, SNCG, IDE, LIPA, TNFRSF6 and/or KNSL1 may correlate with a subject's response, either positive or negative, to a specific therapeutic drug. One or more allelic variants of these genes are correlated with drug response by obtaining genotype and/or haplotype data from various groups of patients in which the drug has been administered. The genotype or haplotype of the subject can then allow a clinician to take a more individualized approach to preventing the onset or progression of neurodegenerative disease by tailoring the therapy to increase the chance of a favorable effect.

Also provided are methods for predicting a response, either positive or negative, of a subject to a drug used to treat neurodegenerative disease, such as Alzheimer's disease, or another neurodegenerative disease by detecting, in the subject, the presence or absence of an allelic variant of one or more polymorphic regions of one or more genes selected from the group consisting of uPA, SNCG, IDE, KNSL, LIPA and/or TNFRSF6. A collection of polymorphic regions that individually represent allelic variants that are associated with neurodegenerative disease including Alzheimer's disease may often be more informative than a single allelic variant for indicating whether an individual will positively respond to a given drug for neurodegenerative disease. Each allelic variant may be assayed individually or simultaneously using multiplex assay methods. Thus, the provided methods encompass detection of an allelic variant of more than one polymorphic region of one or more of a uPA, SNCG, IDE, LIPA, TNFRSF6 and/or KNSL1 gene.

Further provided is a method for predicting a response of a subject to a drug used to treat Alzheimer's disease, comprising detecting the presence or absence of at least one allelic variant of uPA, SNCG, IDE, LIPA, TNFRSF6 or KNSL1; wherein the presence of at least one allelic variant is indicative of a positive response.

For the above methods of predicting drug response, the allelic variant can be: one or more polymorphic regions of the SNCG gene corresponding to SEQ ID NO:73 nucleotide positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200, or the complement thereof; one or more polymorphic regions of the IDE gene corresponding to SEQ ID NO:187 nucleotide positions 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565, or the complement thereof; or of SEQ ID NO:484 nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671,96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124, or the complement thereof; one or more polymorphic regions of the KNSL1 gene corresponding to SEQ ID NO:348 nucleotide positions 300, 1152, 14235, 15104, 20815, 35719, 36738-36739, 41015, 42125, 45083, 45887, 56706, 56887, 58524, 62661 and 63802, or the complement thereof; or of SEQ ID NO:484 nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153, or the complement thereof; one or more polymorphic regions of the LIPA gene corresponding to SEQ ID NO:468 nucleotide positions 1197, 1307-1309, 1841, 1852, 2075, 6063, 6173, 6194, 7820, 25283, 28453-28465, 28543, 28746, 29904, 37861, 39834, 40018, 7219, 8242, 10114, 10606, 10688, 10729, 11559, 12031, 14497, 14729, 21145, 21329; 21404, 21429, 22246, 22354, 22621, 23802 and 25969, or the complement thereof; and one or more polymorphic regions of the TNFRSF6 gene corresponding to SEQ ID NO:403 nucleotide positions 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026, or the complement thereof.

In a particular embodiment for the SNCG allele, the nucleotide of SEQ ID NO:73 at: position 560 is G or A, at position 590 is A or C, at position 617 is C or T, at position 645 is G or A, at position 915 is T or G, at position 987 is C or A, at position 1723 is A or G, at position 1943 is G or C, at position 1950 is G or A, at position 3151 is A or G, at position 3178 is T or C, at position 3189 is T or C, at position 3284 is G or A, at position 3779 is T or position 3779 is deleted, at position 4156 corresponds to a single nucleotide G that is either inserted or not inserted, at position 4276 is T or A, at position 4311 is C or T, at position 4552 is T or A, at position 4976 is C or position 4976 is deleted, at position 4995 is C or G, at position 5019 is C or T, at position 5025 is C or A, at position 5112 is T or A, at position 5136 is T or A, at position 5517 is T or C, at position 2533 is T or G, at position 3371 is A or C, at position 4627 is T or G, at position 4727 is A or G, at position 4813 is A or C, and at position 5200 is G or C.

In a particular embodiment for the IDE allele, the nucleotide of SEQ ID NO:187 at position 2456 is T or G, at position 3279 is T or C, at position 3407 is C or T, at position 42943 is T or C, at position 62498 is T or C, at position 69586 is T or C, at position 107395 is G or A, at position 112114 is G or A, and at position 116662 is T or A; or the complementary nucleotide(s) in SEQ ID NO:484: at position 820 is A or T, at position 7066 is A or G, at position 11758 is T or C, at position 21270 is T or G, at position 22225 is A or T, at position 29294 is C or T, at position 33452 is G or T, at position 33708 is G or A, at position 36982 is C or T, at position 54862 is A or G, at position 77786 is C or A, at position 80594 is G or A, at position 84792 is T or C, at position 84997 is G or T, at position 86682 is C or T, at position 86857 is T or A, at position 88511 is A or G, at position 90437 is G or T, at position 90593 is G or A, at position 91650 is T or C, at position 91870 is G or A, at position 91878 is G or A, at position 92011 is C or T, at position 93618 is T or C, at position 94344 is C or T, at position 94714 is A or G, at position 95671 is A or G, at position 96324 is A or G, at position 97302 is G or A, at position 97370 is G or A, at position 98253 is T or C, at position 98276 is C or T, at position 98385 is A or G, at position 98646 is T or A, at position 98814 is G or A, at position 99597 is C or T, at position 100378 is T or C, at position 101029 is G or A, at position 101265 is C or T, at position 102465 is C or G, at position 103289 is T or G, at position 103967 is C or T, at position 105793 is A or G, at position 106076 is G or T, at position 106453 is C or T, at position 106600 is A or G, at position 106995 is G or A, at position 107851 is C or T, at position 108434 is G or C, at position 109096 is C or T, at position 109399 is C or T, at position 109483 is T or G, at position 110870 is G or A, at position 111189 is A or G, at position 111972 is G or A, at position 112627 is A or T, at position 112629 is A or T, at position 112631 is T or A, at position 113407 is C or G, at position 114444 is C or G, at position 114482 is G or C, at position 115473 is C or position 115473 is deleted, at position 116681 is G or T; at position 117226 is A or T, at position 117600 is A or G, at position 117802 is C or T, at position 118223 is G or C, at position 120011 is C or T, at position 122260 is A or G, at position 123165 is A or G, at position 123424 is G or A, at position 124352 is A or G, at position 124501 is C or T, at position 124692 is A or G, at position 125113 is T or A, at position 125159 is G or A, at position 126568 is G or C, at position 127166 is C or G, at position 127598 is T or C, at position 127600 is T or C, at position 127609 is T or C, at position 127614 is T or C, at position 127623 is T or C, at position 127662 is G or A, at position 128053 is G or A, at position 128261 is a repeat of -TAAA- occurring 6, 7, or 8 times beginning at position 128261, at position 128289 is A or T, at position 128291 is T or G, at position 128393 is T or G, at position 129444 is C or T.

In a particular embodiment for the KNSL1 allele, the nucleotide of SEQ ID NO:348 at position 300 corresponds to a dinucleotide -CA- that is either inserted or not inserted beginning at position 300, at position 1152 is G or T, at position 14235 corresponds to a single nucleotide T that is either inserted or not inserted, at position 15104 is A or G, at position 20815 is T or C, at position 35719 is T or C, at positions 36738-36739 is a dinucleotide corresponding to CA or AC, at position 41015 corresponds to the oligonucleotide -AATTT- that is either inserted or not inserted beginning at position 41015, at position 42125 is T or G, at position 45083 is C or T, at position 45887 is G or C, at position 56706 is C or T, at position 56887 is A or G, at position 58524 is C or T, at position 62661 is C or T, and at position 63802 is A or C, or the nucleotide(s) in SEQ ID NO:484: at position 130876 is T or C, at position 131378 is G or A, at position 131616 is G or A, at position 131620 is G or A, at position 131688 is T or G, at positions 131998-131203 are -CTTTTC- or positions 131998-131203 are deleted, at position 132004 is either a 9, 16, 21, 26, or 29 base pair poly-T repeat beginning at nucleotide 132004, at position 132370 is A or G, at position 132697 is A or G, at position 132968 is C or T, at position 133355 is either a 6, 7 or 8 base pair poly-T repeat beginning at nucleotide 133355, at position 133806 is T or G, at position 134030 is G or A, at position 134291 is A or G, at position 134661 is G or A, at position 137087 is A or G, at position 137142 is G or A, at position 138396 is C or T, at position 140665 is T or G, at position 140736 is A or G, at position 141173 is A or G, at position 142056 is T or C, at position 142777 corresponds to a dinucleotide -AG- that is either inserted or not inserted beginning at position 142777, at position 143025 is G or T, at position 143729 is C or A, at position 144484 is T or A, at position 146181 is T or A, at position 147051 is G or A, at position 147322 is C or T, at position 147707 is G or T, at positions 147842-147845 are -AGTT- or positions 147842-147845 are deleted, at position 148080 is C or T, at position 149026 is either a 17, 18, 19 or 22 base pair -AC- repeat beginning at nucleotide 149026, at position 149044 is either a 22, 24, 28, 30, 32 or 36 base pair -GT- repeat beginning at nucleotide 149044, at position 149389 is A or G, at position 150003 is G or A, at position 150384 is G or T, at position 150454 is C or T, at position 150686 is G or T, at position 151343 is C or T, at position 151961 is C or T, at position 152119 is C or T, at position 153791 is C or G, at position 154328 is A or T, at position 154513 is C or A, at position 154639 is G or A, at position 155049 is T or C, at position 155114 is T or C, at position 158040 is C or A, at position 158895 is G or A, at position 191284 is C or T, at position 192272 is C or T, at position 192698 is A or T, at position 193706 is T or A.

In a further embodiment for the LIPA allele, the nucleotide of SEQ ID NO:468 at position 1197 is C or G, at positions 1307-1309 are ATC or positions 1307-1309 are deleted, at position 1841 is A or C, at position 1852 is G or A, at position 2075 is G or A, at position 6063 is G or T, at position 6173 is A or C, at position 6194 is G or A, at position 7820 is C or G, at position 25283 is G or C, at positions 28453-28465 are -TCCGCGAGAGGGC- or positions 28453-28465 are deleted, at position 28543 is C or T, at position 28746 is A or C, at position 29904 is G or A, at position 37861 is C or T, at position 39834 is T or A, and at position 40018 is C or T.

In a further embodiment for the TNFRSF6 allele, the nucleotide of SEQ ID NO:403 at position 1530 is T or C, at position 1550 is A or G, at position 14525 is G or A, at position 14714 is C or T, at position 18982 is G or C, at position 19069 is A or G, at position 20412 is A or G, at position 20552 is A or G, at position 23199 is G or A, at position 23416 is T or C, at position 24890 is A or G, at position 26359 is A or T, at position 1926 is G or A, at position 2269 is G or A, at position 18934 is C or T, at position 19227 is C or T, and at position 22026 is C or G.

The above-described methods further comprise detecting the presence or absence of at least one allelic variant of a polymorphic region of another gene associated with Alzheimer's disease or neurodegenerative disease, wherein the presence of the two allelic variants is indicative of a positive or negative response.

Another gene associated with neurodegenerative disease or specifically Alzheimer's disease includes, but is not limited to, APOE4.

Detection can be by any suitable method including, but not limited to, sequencing, allele specific hybridization, primer specific extension, oligonucleotide ligation assay, restriction enzyme site analysis and single-stranded conformation polymorphism analysis.

Kits and Solid Supports

Also provided are kits for determining whether a subject has a predisposition for, protection against, and/or the presence of a neurodegenerative disease such as Alzheimer's disease. An exemplary kit for determining whether a subject has a predisposition for or the presence of a neurodegenerative disease, such as Alzheimer's disease, comprises at least one container means having disposed within at least one probe or primer disclosed herein. The kits can also provide at least one other container means having disposed within at least one probe or primer which specifically hybridizes adjacent to or at a polymorphic region of an APOE4 gene. Further provided are kits with instructions for use and/or other reagents useful for carry out detection.

Also provided is a solid support comprising a nucleic acid comprising at least one polymorphic region of an uPA, SNCG, IDE, LIPA, TNFRSF6 or KNSL1 gene and at least one polymorphic region of another gene associated with Alzheimer's disease. The other gene associated with Alzheimer's disease includes, but is not limited to APOE4. The solid support can be a microarray. Preparation of microarrays is well known in the art (see, e.g., U.S. Pat. Nos. 5,837,832; 5,858,659; 6,043,136; 6,043,031 and 6,156,501). Also provided are solid supports, such as microarrays, comprising one or more of the probes or primers disclosed herein.

Transgenic Animals

Further provided are transgenic animals, such as non-human transgenic animals, comprising heterologous nucleic acid encoding human uPA, SNCG, IDE, LIPA, TNFRSF6 or KNSL1 proteins or portion thereof. The heterologous nucleic acid can be either a genomic sequence containing the complete gene or a portion thereof, or the complete cDNA or portion thereof. Heterologous nucleic acid, also encompasses other genes associated with Alzheimer's disease, including, but not limited to APOE. The transgenic nucleic acid is expressed in the animal. Expression in the animal may result in the production of uPA, SNCG, IDE, LIPA, TNFRSF6 or KNSL1 protein and/or one or more biological events characteristic of neurodegenerative diseases, including Alzheimer's disease. Exemplary transgenic animals include, but are not limited to, mammals, including rodents, such as rats, and mice, Drosophila melanogaster (D. melanogaster), Caenorhabditis elegans (C. elegans) and the like.

Exemplary transgenic animals, comprise heterologous nucleic acid encoding a human protein, or portion thereof, selected from the group of human proteins consisting of uPA, SNCG, IDE, KNSL, LIPA and TNFRSF6, wherein the heterologous nucleic acid comprises an allelic variant of one or more polymorphic regions occurring at a nucleotide position corresponding to one or more nucleotide positions, or complements thereof, selected from the group of consisting of: nucleotide positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200 of SEQ ID NO:73; nucleotide positions 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565 of SEQ ID NO:187 or the complement of SEQ ID NO:484 nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124; nucleotide positions 300, 1152, 14235, 15104, 20815, 35719, 36738-36739, 41015, 42125, 45083, 45887, 56706, 56887, 58524, 62661 and 63802 of SEQ ID NO:348 or SEQ ID NO:484 nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153; nucleotide positions 1197, 1307 to 1309, 1841, 1852, 2075, 6063, 6173, 6194, 7820, 25283, 28453 to 28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969 of SEQ ID NO:468; and nucleotide positions 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026 of SEQ ID NO:403.

Methods to Screen for a Biologically Active Agent

Further provided are in vitro and in vivo methods for screening test compounds to identify therapeutics for treating or preventing the development of a neurodegenerative disease, such as Alzheimer's disease. Also provided are methods to screen for biologically active agents that modulate the expression or activity of a uPA, SNCG, IDE, KNSL, LIPA and/or TNFRSF6 protein.

Further provided is a method of screening for an active agent that modulates a biological event characteristic of Alzheimer's disease (AD) in a subject, comprising (a) combining a candidate agent with a transgenic animal comprising a transgenic nucleotide sequence encoding an allelic variant of a uPA, SNCG, IDE, KNSL, LIPA and/or TNFRSF6 gene associated with the manifestation of Alzheimer's disease, stably integrated into the genome of the animal and operably linked to a promoter, wherein when the transgenic nucleic acid is expressed the transgenic animal develops one or more characteristics of Alzheimer's disease; and (b) determining the effect of the agent upon one or more characteristics of Alzheimer's disease.

Also provided are methods as described above, wherein the SNCG allelic variant comprises one or more polymorphic regions occurring at a nucleotide position corresponding to a nucleotide position selected from the group of nucleotide positions of SEQ ID NO:73 consisting of 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200, or the complement thereof; wherein the IDE allelic variant comprises one or more polymorphic regions occurring at a nucleotide position corresponding to a nucleotide position selected from the group of nucleotide positions of SEQ ID NO:187 consisting of 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565, or the complement thereof; or the IDE allelic variant comprises one or more polymorphic regions occurring at a nucleotide position complemetary to a nucleotide position selected from the group of nucleotide positions of SEQ ID NO:484 consisting of 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124, or the complement thereof; wherein the KNSL1 allelic variant comprises one or more polymorphic regions occurring at a nucleotide position corresponding to a nucleotide position selected from the group of nucleotide positions of SEQ ID NO:348 consisting of 300, 1152, 14235, 15104, 20815, 35719, 36738-36739, 41015, 42125, 45083, 45887, 56706, 56887, 58524, 62661 and 63802, or the complement thereof; or the KNSL1 allelic variant comprises one or more polymorphic regions occurring at a nucleotide position corresponding to a nucleotide position selected from the group of nucleotide positions of SEQ ID NO:484 consisting of 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153, or the complement thereof; wherein the LIPA allelic variant comprises one or more polymorphic regions occurring at a nucleotide position corresponding to a nucleotide position selected from the group of nucleotide positions of SEQ ID NO:468 consisting of 1197, 1307 to 1309, 1841, 1852, 2075, 6063, 6173, 6194, 7820, 25283, 28453 to 28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969, or the complement thereof; wherein the TNFRSF6 allelic variant comprises one or more polymorphic regions occurring at a nucleotide position corresponding to a nucleotide position selected from the group of nucleotide positions of SEQ ID NO:403 consisting of 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026, or the complement thereof.

Also provided is a method of screening for biologically active agents that modulate the expression or activity of a uPA, SNCG, IDE, KNSL, LIPA or TNFRSF6 protein, comprising combining a candidate agent with a cell comprising a nucleotide sequence which contains at least a portion of a uPA, SNCG, IDE, KNSL, LIPA or TNFRSF6 allele which is an allelic variant of the uPA, SNCG, IDE, KNSL, LIPA or TNFRSF6 gene, respectively, wherein said portion comprises one or more polymorphic regions occurring at a nucleotide position corresponding to a nucleotide position selected from: SEQ ID NO:73 nucleotide positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200, or the complement thereof; SEQ ID NO:187 nucleotide positions 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565, or the complement thereof, SEQ ID NO:484 IDE nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124, or the complement thereof; SEQ ID NO:348 nucleotide positions 300, 1152, 14235, 15104, 20815, 35719, 36738-36739, 41015, 42125, 45083, 45887, 56706, 56887, 58524, 62661 and 63802, or the complement thereof, or SEQ ID NO:484 nucleotide positions 130876, 131378, 131616; 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153, or the complement thereof; SEQ ID NO:468 nucleotide positions 1197, 1307 to 1309, 1841, 1852, 2075, 6063, 6173, 6194, 7820, 25283, 28453 to 28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969, or the complement thereof; and SEQ ID NO:403 consisting of 1530, 1550, 14525,14714, 18982, 19069, 20412, 20552, 23199, 23416, 24890, 26359, 199, 213, 843, 2967, 3103, 5335, 5345,6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026, or the complement thereof; and operably linked to a promoter such that the nucleotide sequence is expressed as a uPA, SNCG, IDE, KNSL, LIPA or TNFRSF6 protein in the cell; and determining the effect of the agent upon the expression and/or activity of the respective uPA, SNCG, IDE, KNSL, LIPA or TNFRSF6 protein. cDNAS

Provided herein are cDNAs including, among others, an isolated nucleic acid encoding a polymorphic SNCG protein comprising the coding region or full-length of SEQ ID NO:469 having variant nucleotides corresponding to positions: 30, 57, 85, 243, 250, 377, 512, 531, 555, 561 and 672 of SEQ ID NO:469. In a particular embodiment, the isolated nucleic acid encoding a polymorphic SNCG protein, comprises SEQ ID NO:469, wherein the nucleotide at position 672 is not T. In another embodiment, the nucleotide at position 672 of SEQ ID NO:469 is A.

Also provided herein are cDNAs encoding IDE protein comprising the coding region or full-length of SEQ ID NO:470 having a variant nucleotide corresponding to position 7 of SEQ ID NO:470, wherein the nucleotide at position 7 is not C. In a particular embodiment, the nucleotide at position 7 of SEQ ID NO:470 is T.

Also provided herein are cDNAs encoding a polymorphic KNSL1 protein comprising the coding region or full-length of: SEQ ID NO:471 having a variant nucleotide at position 2747 of SEQ ID NO:471; SEQ ID NO:473 having a variant nucleotide at position 2610 of SEQ ID NO:473; SEQ ID NO:475 having a variant nucleotide at position 2695 of SEQ ID NO:475, wherein the variant nucleotide at each of these positions is not C. In a particular embodiment, the nucleotide at position 2747 of SEQ ID NO:471, at position 2610 of SEQ ID NO:473, and at position 2695 of SEQ ID NO:475 is T, which results in a cysteine at amino acid 869 in the translated protein.

Also provided herein are cDNAs encoding a polymorphic TNFRSF6 protein comprising the coding region or full-length of SEQ ID NO:477 having variant nucleotides corresponding to positions 208 and 420 of SEQ ID NO:477. In a particular embodiment, the isolated nucleic acid encoding a polymorphic TNFRSF6 protein, comprises SEQ ID NO:477, wherein the nucleotide at position 208 is not G. In another embodiment, the nucleotide at position 208 of SEQ ID NO:477 is A.

Also provided herein are cDNAs encoding TNFRSF6 protein comprising the coding region or full-length of SEQ ID NO:478 having variant nucleotides corresponding to positions 377, 416, 836 and 1766 of SEQ ID NO:478. In a particular embodiment, the isolated nucleic acid encoding a polymorphic TNFRSF6 protein, comprises SEQ ID NO:478, wherein the nucleotide at position 377 is not G. In another embodiment, the nucleotide at position 377 of SEQ ID NO:478 is A.

Also provide herein are cDNAs encoding TNFRSF6 protein comprising the coding region or full-length of SEQ ID NO:479 having variant nucleotides corresponding to positions 403, 442, 862 and 1792 of SEQ ID NO:479. In a particular embodiment, the isolated nucleic acid encoding a polymorphic TNFRSF6 protein, comprises SEQ ID NO:479, wherein the nucleotide at position 403 is not G. In another embodiment, the nucleotide at position 403 of SEQ ID NO:479 is A.

Also provide herein are cDNAs encoding TNFRSF6 protein comprising the coding region or full-length of SEQ ID NO:480 having variant nucleotides corresponding to positions 208, 247 and 604 of SEQ ID NO:480. In a particular embodiment, the isolated nucleic acid encoding a polymorphic TNFRSF6 protein, comprises SEQ ID NO:480, wherein the nucleotide at position 208 is not G. In another embodiment, the nucleotide at position 208 of SEQ ID NO:480 is A.

Also provide herein are cDNAs encoding TNFRSF6 protein comprising the coding region or full-length of SEQ ID NO:481 having variant nucleotides corresponding to positions 208 and 247 of SEQ ID NO:481. In a particular embodiment, the isolated nucleic acid encoding a polymorphic TNFRSF6 protein, comprises SEQ ID NO:481, wherein the nucleotide at position 208 is not G. In another embodiment, the nucleotide at position 208 of SEQ ID NO:481 is A.

Also provided herein is an isolated nucleic acid encoding a polymorphic LIPA protein comprising the coding region or full-length of SEQ ID NO:482 having variant nucleotides corresponding to positions: 86, 107, 2149, and 2333 of SEQ ID NO:482. In a particular embodiment, the isolated nucleic acid encoding a polymorphic LIPA protein, comprises SEQ ID NO:482, wherein the nucleotide at position 2333 of SEQ ID NO:482 is not C. In another embodiment, the nucleotide at position 2333 of SEQ ID NO:482 is T.

Methods for Detecting Altered Levels of Risk

Also provided are methods for detecting an altered level of risk for neurodegenerative disease, such as AD, in a subject, comprising:
the step of detecting in a target nucleic acid obtained from the subject the presence of an allelic variant of one or more polymorphic regions of one or more genes selected from the group consisting of uPA, SNCG, IDE, KNSL, LIPA and TNFRSF6, wherein the presence of at least one of the allelic variant of one or more polymorphic regions is indicative of an altered level of risk for the neurodegenerative disease compared to a subject not having the allelic variant.

In one embodiment, the altered level of risk corresponds to a predisposition for a neurodegenerative disease. In another embodiment, the altered level of risk corresponds to protection from the neurodegenerative disease. Exemplary polymorphic regions and particular allelic variants for use in these methods include those set forth in the Examples at Tables 2, 4 and 4-B, 6 and 6-B, 8, 10 12 and 12-B as well as Tables A-F.

Methods for Treating a Subject Manifesting an AD Phenotype

Further provided are methods of treating a subject manifesting an Alzheimer's disease phenotype. Certain ambiguous phenotypes, e.g., dementia, manifested in AD also occur in connection with other diseases and conditions which may be treated using drugs and other treatments that are different from drugs and methods used to treat AD. Genotyping of chromosome 10 polymorphic regions described herein, and optionally other AD-associated markers, in subjects manifesting such an AD phenotype(s) permits confirmation of AD diagnoses and assists in distinguishing between AD and other possible diseases or disorders. Once an individual is genotyped as having or being predisposed to AD, he or she may be treated with any known methods effective in treating AD.

Accordingly, methods provided herein of treating a subject manifesting an Alzheimer's disease phenotype, include steps of (a) detecting in nucleic acid obtained from the subject the presence or absence of an allelic variant of one or more polymorphic regions of one or more genes associated with AD selected from the group consisting of uPA, SNCG, IDE, KNSL1, LIPA and TNFRSF6, wherein the presence of at least one of said allelic variant of one or more polymorphic regions is indicative of the occurrence of AD, and (b) selecting a treatment plan that is effective for treatment of Alzheimer's disease. In particular embodiments of these methods, the presence or absence of a particular allelic variant is detected at one or more of polymorphic regions (e.g., SNPs and the like) listed herein throughout the specification, including in Tables 2, 4 and 4-B, 6 and 6-B, 8, 10, 12 and 12-B and Tables A through F, as well as FIGS. 1 through 10. In further embodiments of these methods, the one or more polymorphic regions of the SNCG occurs at a nucleotide position corresponding to a nucleotide position selected from the group of nucleotide positions of SEQ ID NO:73 consisting of 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200, or the complement thereof; the one or more polymorphic regions of the IDE gene occurs at a nucleotide position corresponding to a nucleotide position selected from the group of nucleotide positions of SEQ ID NO:187 consisting of 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565, or the complement thereof, or of SEQ ID NO:484 IDE consisting of 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124, or the complement thereof; the one or more polymorphic regions of the KNSL1 gene occurs at a nucleotide position corresponding to a nucleotide position selected from the group of nucleotide positions of SEQ ID NO:348 consisting of 300, 1152, 14235, 15104, 20815, 35719, 36738-36739, 41015, 42125, 45083, 45887, 56706, 56887, 58524, 62661 and 63802, or the complement thereof or of SEQ ID NO:484 consisting of 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153, or the complement thereof; the one or more polymorphic regions of the LIPA gene occurs at a nucleotide position corresponding to a nucleotide position selected from the group of nucleotide positions of SEQ ID NO:468 consisting of 1197, 1307 to 1309, 1841, 1852, 2075, 6063, 6173, 6194, 7820, 25283, 28453 to 28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969, or the complement thereof; the one or more polymorphic regions of the TNFRSF6 gene occurs at a nucleotide position corresponding to a nucleotide position selected from the group of nucleotide positions of SEQ ID NO:403 consisting of 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026, or the complement thereof.

Also provided are methods for detecting the presence or absence of a polymorphism of a uPA, SNCG, IDE, KNSL1, TNFRSF6 or LIPA gene. These methods can include a step of determining the presence or absence of an insertion or deletion, and/or determining the identity of the nucleotide at and/or surrounding a position corresponding to a nucleotide position selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600,.127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715.

Further provided are methods for identifying a polymorphism or combination of polymorphisms associated with neurodegenerative disease. Such methods can include a step of testing one or more polymorphisms in a uPA, SNCG, IDE, KNSL1, TNFRSF6 or LIPA gene individually and/or in combinations for genetic association with a neurodegenerative disease.

Methods for identifying a polymorphism or combination of polymorphisms associated with a uPA, SNCG, IDE, KNSL1, TNFRSF6 or LIPA-mediated disease or disorder are also provided. Such methods can include a step of testing one or more polymorphisms in a urokinase plasminogen activator gene, SNCG gene, IDE gene, KNSL1 gene, TNFRSF6 gene and/or LIPA gene individually and/or in combinations for genetic association with a uPA, SNCG, IDE, KNSL1, TNFRSF6 or LIPA-mediated disease or disorder, respectively, wherein the one or more polymorphisms occurs at nucleotide positions corresponding to a nucleotide position selected from the group consisting of:
a SNCG nucleotide position of SEQ ID NO:73, or the complement thereof, selected from the group consisting of nucleotide positions 915,-987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the-group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715.

Also provided are methods for detecting the presence or absence in a subject of one or more polymorphisms associated individually and/or in combination with a neurodegenerative disease or disorder which include a step of detecting in nucleic acid obtained from the subject the presence or absence of one or more polymorphisms in the uPA, SNCG, IDE, KNSL1, TNFRSF6 or LIPA gene, wherein individually and/or in combination the polymorphisms are associated with a neurodegenerative disease or disorder. The neurodegenerative disease or disorder can be Alzheimer's disease. The Alzheimer's disease can be a disease with onset ages of greater than or equal to about 50 years, or greater than or equal to about 60-years, or greater than or equal to about 65 years. The association between the one or more polymorphisms and Alzheimer's disease can be such that it yields a positive result in a family-based test for association. In particular methods, the positive result is a P value less than or equal to 0.05. In one embodiment, the positive result is a P value less than 0.05. In some embodiments, the P value is a value obtained after correction in which the probability value required to give significance is divided by the number of tests conducted.

Methods for detecting the presence or absence in a subject of a polymorphism or a combination of polymorphisms that is associated with a uPA, SNCG, IDE, KNSL1, TNFRSF6 or LIPA-mediated disease or disorder are also provided. These methods can include a step of detecting in nucleic acid obtained from the subject the presence or absence of one or more polymorphisms in a uPA, SNCG, IDE, KNSL1, TNFRSF6 or LIPA gene, wherein the one or more polymorphisms occurs at nucleotide positions corresponding to a nucleotide position selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 1064, 106600, 106995, 107851, 108434, 109096, 109399, 109483, 110870, 111189, 111972, 112627, 112629, 1112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715.

In particular embodiments of these methods, the neurodegenerative disease or disorder is Alzheimer's disease. The Alzheimer's disease can be a disease with onset ages of greater than or equal to about 50 years, or greater than or equal to about 60 years, or greater than or equal to about 65 years. The association between the one or more polymorphisms and Alzheimer's disease can be such that it yields a positive result in a family-based test for association. In particular methods, the positive result is a P value less than or equal to 0.05. In one embodiment, the positive result is a P value less than 0.05. In some embodiments, the P value is a value obtained after correction in which the probability value required to give significance is divided by the number of tests conducted.

Also provided are methods for determining a predisposition to or the occurrence of a uPA, SNCG, IDE, KNSL1, TNFRSF6 or LIPA-mediated disease or disorder in a subject. These methods can include a step of:
detecting in a nucleic acid obtained from the subject the presence or absence of one or more polymorphisms of a uPA, SNCG, IDE, KNSL1, TNFRSF6 or LIPA gene, wherein the one or more polymorphisms occur at nucleotide positions corresponding to nucleotide positions selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758,21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715;
wherein the presence of at least one polymorphism is indicative of a predisposition to or the occurrence of a uPA, SNCG, IDE, KNSL1, TNFRSF6 or LIPA-mediated disease or disorder.

Methods for predicting a response of a subject to an agent used to treat a uPA, SNCG, IDE, KNSL1, TNFRSF6 or LIPA-mediated disease are further provided. These methods can include a step of:
detecting in nucleic acid obtained from the subject the presence or absence of one or more polymorphisms of a uPA, SNCG, IDE, KNSL1, TNFRSF6 or LIPA gene that occur at nucleotide positions corresponding to nucleotide positions selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982,54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93and714-715;
wherein the presence of the one or more polymorphisms, individually and/or in combination, is indicative of an increased or decreased likelihood that the treatment will be effective.

Also provided are methods of screening for an agent that modulates Aβ protein levels which can include a step of:
(a) combining a candidate agent with a cell and/or animal comprising nucleic acid comprising a uPA, SNCG, IDE, KNSL1, TNFRSF6 or LIPA gene and/or a portion or portions thereof, that encodes a uPA, SNCG, IDE, KNSL1, TNFRSF6 or LIPA protein and comprises one or more polymorphisms of a uPA, SNCG, IDE, KNSL1, TNFRSF6 or LIPA gene, wherein the cell and/or animal produces Aβ protein; and
(b) determining the effect of the agent upon Aβ protein levels in the animal and/or cell and/or extracellular medium.

Methods of screening for an agent that modulates the expression and/or activity of a uPA, SNCG, IDE, KNSL1, TNFRSF6 or LIPA protein provided herein can include a step of:
(a) combining a candidate agent with a cell and/or animal comprising nucleic acid that encodes a uPA, SNCG, IDE, KNSL1, TNFRSF6 or LIPA protein or reporter molecule operatively linked to one or more portions of a uPA, SNCG, IDE, KNSL1, TNFRSF6 or LIPA gene comprising one or more polymorphisms of a uPA, SNCG, IDE, KNSL1, TNFRSF6 or LIPA gene, wherein the one or more polymorphisms occur at nucleotide positions corresponding to nucleotide positions selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715; and
(b) determining the effect of the agent on the expression and/or activity of uPA, SNCG, IDE, KNSL1, TNFRSF6 or LIPA mRNA, uPA, SNCG, IDE, KNSL1, TNFRSF6 or LIPA protein or the reporter molecule encoded by the nucleic acid in the cell and/or animal.

Methods of screening for an agent that modulates the expression and/or activity of a uPA, SNCG, IDE, KNSL1, TNFRSF6 or LIPA protein provided herein can include a step of:
(a) combining a candidate agent with a cell and/or animal comprising nucleic acid comprising a uPA, SNCG, IDE, KNSL1, TNFRSF6 or LIPA gene and/or a portion or portions thereof that encodes a-uPA, SNCG, IDE, KNSL1, TNFRSF6 or LIPA protein comprising one or more polymorphisms of a uPA, SNCG, IDE, KNSL1, TNFRSF6 or LIPA gene, wherein the one or more polymorphisms occur at nucleotide positions corresponding to nucleotide positions selected from the group of nucleotide positions of the IDE gene corresponding to nucleotides 2456, 327.9, 3407 and 42943 of SEQ ID NO:187, or the complementary positions thereof; of the KNSL1 gene corresponding to nucleotides 132370, 133355, 147842 and 178981 of SEQ ID NO:484, or the complementary positions thereof; of the LIPA gene corresponding to nucleotides 1852, 6063 and 7820 of SEQ ID NO:468; of a uPA gene or cDNA corresponding to nucleotide positions 3169, 3947, and 6532 of SEQ ID NO:559 or 560; and the complementary positions thereof; and
(b) determining the effect of the agent upon the expression and/or activity of uPA, SNCG, IDE, KNSL1, TNFRSF6 or LIPA mRNA and/or uPA, SNCG, IDE, KNSL1, TNFRSF6 or LIPA protein encoded by the nucleic acid in the cell and/or animal. Similar to the polymorphisms in Table 14 herein that are in linkage disequilibrium with SNP 122260 of SEQ ID NO:484, the polymorphisms that are in linkage disequilibrium with position 178980/178981 of SEQ ID NO:484 (a position associated with AD) are also associated with AD, and may therefore be assessed in place of assessing the polymorphism at position 178980/178981 of SEQ ID NO:484 for each of the methods and applications provided herein that assess the polymorphism at position 178980/178981 of SEQ ID NO:484.

In methods of screening for an agent that modulates the expression of a uPA, SNCG, IDE, KNSL1, TNFRSF6 or LIPA protein, a step can include:
(a) combining a candidate agent with a recombinant cell and/or transgenic animal comprising nucleic acid encoding a reporter molecule operatively linked to one or more portions of a uPA, SNCG, IDE, KNSL1, TNFRSF6 or LIPA gene sufficient to promote expression of the reporter molecule and comprising one or more polymorphisms of a uPA, SNCG, IDE, KNSL1, TNFRSF6 or LIPA gene, wherein the one or more polymorphisms occur at nucleotide positions corresponding to nucleotide positions selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997,86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715; and
(b) determining the effect of the agent on the expression and/or activity of the reporter molecule in the cell and/or animal.

Also provided are methods for confirming a phenotypic diagnosis of Alzheimer's disease in a subject. These methods can include a step of:
detecting in nucleic acid obtained from a subject diagnosed with Alzheimer's disease the presence or absence of one or more polymorphisms of a uPA, SNCG, IDE, KNSL1, TNFRSF6 or LIPA gene, wherein the presence of the one or more polymorphisms, individually and/or in combination, confirms a phenotypic diagnosis of Alzheimer's disease.

Methods for determining a level of risk for a uPA, SNCG, IDE, KNSL1, TNFRSF6 or LIPA-mediated disease or disorder in a subject provided herein can include a step of:
detecting in nucleic acid obtained from the subject the presence or absence of one or more polymorphisms in a uPA, SNCG, IDE, KNSL1, TNFRSF6 or LIPA gene, wherein the one or more polymorphisms occur at nucleotide positions corresponding to nucleotide positions selected from the group consisting of:
a SNCG nucleotide position of SEQ ID NO:73, or the complement thereof, selected from the group consisting of nucleotide positions 915, 987, 2533, 3151, 3178, 3189, 3284, 3371, 3779, 4156, 4276, 4311, 4627, 4727, 4813, 5136, 5200 and 5517;
an IDE nucleotide position of SEQ ID NO:187, or the complement thereof, selected from the group consisting of nucleotide positions 2456, 3279, 3407, 42943, 62498, 69586, 107395 and 112114; or a nucleotide position of SEQ ID NO:484, or the complement thereof, selected from the group consisting of nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444;
a KNSL1 allele spanning a nucleotide position of SEQ ID NO:348, or the complement thereof, selected from the group consisting of nucleotide positions 300, 1152, 14235, 15104, 20815, 36738-36739, 41015, 42125, 56706, 56887 and 58524; or a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706;
a LIPA nucleotide position of SEQ ID NO:468, or the complement thereof, selected from the group consisting of nucleotide positions 1197, 7820, 28543 and 28746;
a TNFRSF6 nucleotide position of SEQ ID NO:403, or the complement thereof, selected from the group consisting of nucleotide positions 1530, 14525, 14714, 19069, 20412, 20552, 23199, 23416, 1926 and 2269; and
a uPA nucleotide position of SEQ ID NO:569 or 560, or the complement thereof, selected from the group consisting of 401, 515, 748 and 1752; and of SEQ ID NO:563, or the complement thereof, consisting of 93 and 714-715.

### BRIEF DESCRIPTION OF THE DRAWINGS

FIG. 1 shows a genomic DNA sequence corresponding to GenBank Accession No. AF037207 (SEQ ID NO:72) containing the SNCG gene therein. Exons are indicated by ˜, and the polymorphic regions set forth in Table 2 are labelled accordingly. Previously identified SNPs are indicated by an “rs” preceding a numerical value.

FIG. 2 shows a genomic DNA sequence corresponding to the reverse complement of NCBI Accession No. AL356128.15 (SEQ ID NO:186) containing the IDE gene therein. Exons are indicated by ˜, and the polymorphic regions set forth in Table 4 are labelled accordingly.

FIG. 3 shows a genomic DNA sequence corresponding to the reverse complement of a 63,834 nucleotide portion (SEQ ID NO:347) of NCBI Accession No. NT_{—}008769.1 starting at nucleotide 1,669,312 and ending at nucleotide 1,733,136, having the human KNSL1 gene therein. Exons are indicated by ˜, and the polymorphic regions set forth in Table 6 are labelled accordingly. Previously identified SNPs are indicated by an “rs” preceding a numerical value.

FIG. 4 shows a genomic DNA sequence corresponding to the reverse complement of a 28,118 nucleotide portion (SEQ ID NO:402) of NCBI Accession No. AL 157394.11 starting at nucleotide 17,215 and ending at nucleotide 45,332, having the human TNFRSF6 gene therein. Exons are indicated by ˜, and the polymorphic regions set forth in Table 8 are labelled accordingly.

FIG. 5 shows a genomic DNA sequence corresponding to the 40,178 nucleotide portion (SEQ ID NO:467) of NCBI Accession No. NT_{—}008679.5 starting at nucleotide 6,017,146 and ending at 6,057,323, having the human LIPA gene therein. Exons are indicated by ˜, and the polymorphic regions set forth in Table 10 are labelled accordingly. Previously identified SNPs are indicated by an “rs” preceding a numerical value.

FIG. 6 shows a genomic DNA sequence corresponding to the IDE/KNSL1 genes taken from the human genome hg12 draft build of chromosome 10:93094801 to 93296900 (SEQ ID NO:484) available from “www.genome.ucsc.edu”. This sequence is also contained in NCBI Contig NT 008769. This sequence (SEQ ID NO:484) has the complement of the human IDE gene in reverse 3′ to 5′ orientation corresponding to approximately nucleotides 1-130,000; the human KNSL gene in 5′ to 3′ orientation, and the respective intergenic region therein. Exons are indicated by ˜, and the IDE and KNSL polymorphic regions set forth in Tables 4, 4-B and 6, 6-B, respectively, are labelled accordingly. Previously identified SNPs are indicated by an “rs” preceding a numerical value.

FIG. 7 shows the primers and cycling conditions used for testing the particular IDE and KNSL1 polymorphic regions indicated as set forth in Example 3.

FIG. 8 shows a genomic DNA sequence corresponding to nucleotides 827 to 9141 of Genbank Accession No. AF377330 (SEQ ID NO:559) containing a human uPA (PLAU) gene therein. Exons are indicated by ˜, and the polymorphic regions set forth in Table 12 are labelled accordingly. Previously identified polymorphisms set forth in Table F are indicated by an “rs” preceding a numerical value.

FIG. 9 shows a cDNA sequence corresponding to Genbank Accession No. NM_{—b 002658} (SEQ ID NO:561) encoding a human uPA protein. Polymorphic regions as set forth in Tables 12 and F that are located in this sequence are indicated.

FIG. 10 shows the reverse complement of the nucleotides 74623356-74624256 on Chromosome 10 (SEQ ID NO:563) from the Human Genome Draft build hg12 which is available at www.genome.ucsc.edu.

### DETAILED DESCRIPTION OF PREFERRED EMBODIMENTS

A. Definitions

Unless defined otherwise, all technical and scientific terms used herein have the same meaning as is commonly understood by one of skill in the art to which the invention(s) belong. All patents, patent applications, published applications and publications, published nucleic acid and amino acid sequences, e.g., NCBI and Genbank sequences, other databases, websites and other published materials referred to throughout the entire disclosure herein, unless noted otherwise, are incorporated by reference in their entirety. Where reference is made to a URL or other such identifier or address, it understood that such identifiers can change and particular information on the internet can come and go, but equivalent information can be found by searching the internet. Reference thereto evidences the availability and public dissemination of such information. In the event that there are a plurality of definitions for terms herein, those in this section prevail.

As used herein, sequencing, with reference to nucleic acids, refers to the process of determining a nucleotide sequence and can be performed using any method known to those of skill in the art. For example, if a polymorphism is identified or known, and it is desired to assess its frequency or presence in nucleic acid samples taken from a subject, the region of interest from the samples can be isolated, such as by PCR or restriction fragments, hybridization or other suitable method known to those of skill in the art, and sequenced. For purposes herein, sequencing may be performed using any known method, such as set forth in U.S. Patent Nos. 5,547,835; 5,622,824; 5,851,765; 5,928,906; 5,503,980; 5,631,134; 5,795,714; 5,525,464; 5,695,940; 5,834,189; 5,869,242; 5,876,934; 5,908,755; 5,912,118; 5,952,174; 5,976,802; 5,981,186; 5,998,143; 6,004,744; 6,017,702; 6,018,041; 6,025,136; 6,046,005; 6,087,095; 6,117,634, 6,013,431, WO/98/30883; WO/98/56954; WO/99/09218; WO/00/58519, and the others.

As used herein, “determining a predisposition for or occurrence of a disease or disorder” means that a subject having a particular genotype and/or haplotype (such as a subject who is homozygous or heterozygous with respect to a particular genotype and/or haplotype of a particular allele) has a higher likelihood or increased risk or a statistically significant higher frequency of occurrence for developing or having a particular disease or disorder than one not having such a genotype and/or haplotype. It is also meant to include subjects that already may have symptoms manifested by the disease and can be used to confirm the diagnosis of the particular disease, along with other factors. This may be especially useful in the differential diagnosis of AD and other diseases and disorders that are characterized by similar symptoms of dementia. A statistically significant higher frequency of occurrence of a disease or condition in an individual carrying an allele, genotype or haplotype is relative to the frequency of occurrence of the disease or condition in a member of the same population not carrying the allele, genotype or haplotype. Those skilled in the art are familiar with various tests used to determine statistical significance.

As used herein, the term “subject” refers to mammals and in particular human beings.

As used herein, “target nucleic acid” refers to a nucleic acid molecule which contains all or a portion of a polymorphic region of a nucleic acid segment, for example, a gene or portion thereof, of interest.

As used herein, “genetic marker” refers-to a segment of DNA with an identifiable location on a chromosome. The DNA segment may contain one or more than one nucleotide. The inheritance of a genetic marker may be followed. Typically, genetic markers useful in genetic analyses are polymorphic such that two or more alternative forms or sequences or alleles exist in a population.

As used herein, “polymorphism” refers to the coexistence of more than one form or allele of a nucleic acid, such as a chromosome, or portion thereof, or a gene or portion thereof. For example, a portion or locus of a gene at which there are at least two different alleles, i.e., two different nucleotide sequences, is referred to as a “polymorphic region of a gene.” A polymorphic region can be a single nucleotide or can be several nucleotides in length. Polymorphism includes substitutions, insertions, duplications and deletions of nucleotides. A polymorphism can also refer to a particular nucleotide(s) or nucleotide sequence occurring at a particular polymorphic site.

As used herein, “allele”, which is used interchangeably herein with “allelic variant” refers to alternative forms of a nucleic acid such as a gene or polymorphic regions thereof. Alleles occupy the same locus or position (referred to herein as a polymorphic region) on homologous chromosomes. When a subject has two identical alleles of a polymorphic region within a gene, the subject is said to be homozygous for the allele. When a subject has two different alleles of a polymorphic region within a gene, the subject is said to be heterozygous for the allele. Alleles of a specific gene can differ from each other at a polymorphic region corresponding to a single nucleotide, or several nucleotides, and can include substitutions, deletions, insertions and duplications of nucleotides. An allele of a gene can also be a form of a gene containing a mutation.

As used herein, “genotype” refers to the identity of the alleles present in an individual or sample. The term “genotyping” a sample or individual refers to determining a specific allele or specific nucleotide(s) carried by an individual at particular region(s).

As used herein, “haplotype” refers to a collection of genetic markers. A haplotype can be a combination of alleles present in an individual or sample. A haplotype can be the alleles of different genes received by an individual from one parent, or the array of polymorphisms on a chromosome or portion thereof.

As used herein, the term gene” or “recombinant gene” refers to the segment of DNA involved in producing a functional gene product, including regions preceding (leader) and following (trailer) the region of a gene that contains coding sequences, and may include intervening sequences (introns) between individual coding segments (exons). Because the boundaries between, for example, an upstream, or leader, region of one gene and the upstream (or downstream) region of another gene may not be well-defined, or may overlap, include intergenic sequence and/or be non-existent, the term “gene” when used herein can include nucleotide sequence in the regions surrounding the clearly identified genomic sequence of a gene.

As used herein, the term “coding sequence” refers to that portion of a gene that encodes an amino acid sequence of a protein. A “coding nucleotide sequence,” in the context of a coding nucleotide sequence operatively linked to a promoter, refers to any sequence that encodes a peptide or polypeptide, such as a sequence that encodes all or a portion of KNSL1 or IDE protein, or a sequence that encodes a reporter molecule.

As used herein, the term “reporter molecule” refers to any molecule whose expression and/or activity can be detected, and optionally quantified, when appended to appropriate expression elements. Examples of reporter molecules include enzymes, drug resistance molecules, fluorescent and bioluminescent molecules, and the like. Specific examples of reporter molecules include green fluorescent protein, beta-lactamase, chloramphenicol acetyltransferase and beta-galactosidase.

As used herein, “indicative of a predisposition to a disease” with reference to a particular polymorphism(s) or allele of one or more polymorphic regions means that an individual who possesses the particular allele(s) is more likely to develop (or has a higher risk of developing) or have the disease, e.g., AD, without detectable symptoms thereof than someone who does not have the particular alleles(s). The allele(s) may be over-represented in frequency in individuals with the disease as compared to individuals who do not have the disease. Thus, the particular allele(s) of one or more polymorphic regions can be used to predict disease even in pre-symptomatic or pre-diseased individuals.

As used herein, “indicative of the occurrence of a disease” with reference to a particular allele of one or more polymorphic regions means that an individual who possesses the allele(s) and manifests one or more symptoms of a disease, e.g., AD, is more likely to have the disease than someone who does not have the particular allele(s) and either does or does not manifest one or more symptoms of the disease. Thus, the particular allele(s) may be used to diagnose disease, in particular, differentially diagnose AD. This may be especially useful in the differential diagnosis of diseases and disorders that are characterized by similar symptoms. For example, the particular allele(s) of one or more polymorphic regions may be used to distinguish an individual with AD-associated dementia from an individual with dementia resulting from a condition unrelated to AD. This is particularly of use in diagnosis of AD in individuals about age 50 or greater, about age 60 or greater or about age 65 or greater. In methods of using an allele of one or more polymorphic regions to diagnose a disease, e.g., AD, determination of the presence or absence of the allele(s) in an individual may be conducted in conjunction with other diagnostic tests for the disease, including a variety of neuropsychological tests known to those of skill in the art and referred to herein. A statistically significant higher frequency of occurrence of a disease or condition in subjects carrying an allele, genotype or haplotype can be relative to the frequency of occurrence of the disease or condition in a member of the same or matched population not carrying the allele, genotype or haplotype. Those skilled in the art are familiar with various tests used to determine statistical significance.

As used herein, a “uPA-mediated disease or disorder” refers to a disease or disorder involving a uPA gene, transcript and/or protein. For example, the disease or disorder may be caused, in whole or in part, or exacerbated by uPA protein activity such as enzymatic, proteolytic and/or binding activity. The disease or disorder may involve an aberrant level of uPA gene expression, gene product synthesis and/or aberrant gene product activity relative to levels and/or activities found in normal individuals who are not affected by the disease or disorder. For example, because a uPA-mediated disease or disorder can involve aberrant uPA protein activity and/or uPA protein levels, the disease or disorder may involve proteolysis and/or interactions between uPA and other molecules, e.g., uPAR and PAIs, and alterations therein.

As used herein, “neurodegenerative disease” refers to diseases or disorders wherein selective neuronal populations are destroyed and include Alzheimer's disease (AD), Parkinsonian syndromes such as Parkinson's disease (PD), Huntington's disease (HD), and Prion diseases.

As used herein, an “Alzheimer's disease” or “AD” refers to a group of visible, detectable or otherwise measurable properties characteristic of AD. Exemplary properties include, but are not limited to, dementia, aphasia (language problems), apraxia (complex movement problems), agnosia (problems in identifying objects), progressive memory impairment, disordered cognitive function, altered behavior, including paranoia, delusions and loss of social appropriateness, progressive decline in language function, slowing of motor functions such as gait and coordination in later stages of AD, amyloid-containing plaques which are foci of extracellular amyloid-β (Aβ) protein deposition with dystrophic neurites and associated axonal and dendritic injury and microglia expressing surface antigens associated with activation (e.g., CD45 and HLA-DR), diffuse (“preamyloid”) plaques and neuronal cytoplasmic inclusions such as neurofibrillary tangles containing hyperphosphorylated tau protein or Lewy bodies (containing α-synuclein). Standardized clinical criteria for the diagnosis of AD have been established by NINCDS/ADRDA (National Institute of Neurological and Communicative Disorders and Stroke/Alzheimer's Disease and Related Disorders Association) (McKhann et al. (1984) Neurology 34:939-944). The clinical manifestations of AD as set forth in these criteria are included within the definition of AD. For example, dementia may be established by clinical exam and documented by any of several neuropsychological tests, including the Mini Mental State Exam (MMSE) (Folstein and McHugh (1975) J. Psychiatr. Res. 12:196-198; Cockrell and Folstein (1988) Psychopharm. Bull. 24:689-692), the Blessed Test (Blessed et al. (1968) Br. J. Psychiatry 114:797-811), and the Alzheimer's Disease Assessment Scale-Cognitive (ADAS-COG) Test (Rosen et al. (1984) Am. J. Psychiatry 141:1356-1364; Weyer et al. (1997) Int. Psychogeriatr. 9:123-138; and Ihl et al. (2000) Neuropsychobiol. 4:102-107). A particular form of AD contemplated for diagnosis by the methods and compositions provided herein is late-onset Alzheimer's disease.

As used herein, “late-onset Alzheimer's disease” refers to a type of AD in which AD-associated symptoms manifest at an age of ≧ about 50 years. In late-onset AD, such symptoms may manifest at any age of about 50 years or older and typically may manifest at > about 60 years or > about 65 years.

As used herein, “detecting” or “assessing” in the context of detecting or assessing the presence or absence of a polymorphism or an allelic variant, refers to any method by which the presence or absence of a particular allelic variant or polymorphism can be assessed or determined. The presence or absence of a particular polymorphism can be, for example, the presence or absence of an insertion of one or more nucleotides, a deletion of one or more nucleotides, or the presence of a particular nucleotide versus one of the other nucleotides at a particular polymorphic region. Assessing the presence of a polymorphism can thus be, for example, an assessment of whether a particular nucleotide is present at a polymorphic site, or an assessment of whether an insertion of a nucleotide(s), a deletion of a nucleotide(s) is present at a particular polymorphic site. Exemplary methods that can be used in assessing the presence of or detecting the presence or absence of a polymorphism include, but are not limited to, sequencing, allele specific hybridization, primer specific extension, oligonucleotide ligation assay, restriction enzyme analysis and single-stranded conformation analysis.

As used herein, “pedigree” refers to a family for which information concerning the ancestral relationships and transmission of genetic traits over several generations is known.

As used herein, “linkage disequilibrium” with reference to the relationship between alleles refers to the deviation from the random occurrence of the alleles in a haplotype in populations. Alleles observed together on a chromosome more often than expected from their frequencies in the population may be referred to as in linkage disequilibrium. Alleles that are physically close are more likely to be inherited together than are alleles that are farther apart. Therefore, variations of several markers that are close to, or within, a particular gene variant on a chromosome are likely to be inherited together with that gene variant when they are in linkage disequilibrium. Thus, genetic markers, e.g., microsatellite markers and SNP variations, that are in linkage disequilibrium and associated with a disease phenotype can mark the position on the chromosome in which a susceptibility gene is located.

Generally, linkage disequilibrium spans chromosome segments ranging in size from about ≦5 kb to about 500 kb or less, such as distances of ≦80 kb or ≦50 kb [Risch (2000) Nature 405:847-856; Abecasis et al. (2001) Am. J. Hum. Genet. 68:191-197; Reich et al. (2001) Nature 41:199-2041. It is common, however, to find some degree of linkage disequilibrium between alleles that are up to about 1-2 cM apart. Significant linkage disequilibrium between microsatellite loci has been reported to extend to ≧4 cM [Huttley et al. (1999) Genetics 152:1711-17221 and as great as ≦˜21 cM [Wilson and Goldstein (2000) Am. J. Hum. Genet. 67:926-935]. The degree of linkage disequilibrium between two alleles can vary based on location within the genome, population distribution, population frequency and demographic history [Reich et al. (2001) Nature 41:199-204; Stephens et al. (2001) Science 293:489-493; Wilson and Goldstein (2000) Am. J. Hum. Genet. 67:926-935].

When a disease-causing allele is in linkage disequilibrium with another allele, the frequency of the other allele will be increased in a disease population as compared to a trait-negative population. This increased frequency is referred to as “genetic association” or “allelic association” between the other allele and the disease. Thus, association between a disease trait and a marker allele can be indicative of linkage disequilibrium between the disease-causing allele and the marker allele. Similarly, when an allele that confers protection against a disease is in linkage disequilibrium with another allele, the frequency of the other allele may be increased in a trait-negative population relative to a disease population. This increased frequency is referred to as genetic association between the other allele and the protective allele.

Studies of genetic association are commonly used to identify genes involved in complex traits. Genetic association studies assess correlations between genetic variants and trait differences on a population scale. In association-based methods of mapping genes that increase susceptibility to disease, evidence is sought for a statistically significant association between an allele and a trait or trait-causing allele. The occurrence of a disease-causing allele may be presumed by the occurrence of the disease trait. In such studies, it may turn out that a significant association is obtained between an allele and a trait-negative population. Such an association may be indicative of linkage disequilibrium between that allele and a protective allele that decreases susceptibility to disease. Association studies focus on population frequencies and explore the relationships among frequencies for sets of alleles between loci. Association may be determined using a number of analytical methods, including but not limited to case-control studies, family-based association techniques and haplotype analyses. Association determinations utilizing alleles that are not transmitted from parents to affected individuals as controls and/or related disease family members (e.g., sib pairs) as affected individuals are particularly useful in accurate determination of association. Such determinations are in contrast to association studies using unrelated populations of subjects and matched controls (e.g., case-control studies), which have the advantage of being relatively simple in terms of the sample sets and statistical analyses involved but may be more susceptible to false positive (type 1) and false negative (type l!) errors. Thus, in case-control studies, it is possible in some instances (e.g., population stratification, insufficient sample size and/or poorly matched control groups) to observe association in the absence of linkage disequilibrium.

The terms “recombination fraction” and “recombination frequency” are used herein interchangeably and refer to the probability of a recombination event between two loci in a genome.

As used herein, “linked” refers to a relationship between two loci in a genome. For example, it may refer to the relationship between a polymorphic or marker site on a chromosome and a gene, such as, for example, a gene associated with a disease, such as, for example AD. The relationship may be defined in a number of ways. For example, the relationship may be defined in terms of the extent to which recombination between the loci occurs. Typically, the transmission of alleles located on different chromosomes occurs in a random fashion through independent assortment. Loci representative of two such alleles are considered to be unlinked. If two loci are situated on the same chromosome, the transmission of alleles of one locus may be affected by the presence of the other locus such that the ratios of alleles are no longer independent, and the loci are referred to as “linked.” Two loci are completely linked when there is no recombination between them; the same alleles or phenotypes are always transmitted together from generation to generation within a family. An intermediate state of linkage, referred to as “incomplete linkage” occurs when the transmission of alleles of two loci deviates consistently and measurably from independent assortment but a consistent recombination fraction nonetheless exists for the loci.

Linkage is commonly assessed by the LOD (logarithm of an odds ratio) score method or other acceptable statistical linkage determination. Positive LOD scores can be considered as evidence of linkage between two loci. The greater the LOD score, the greater the possibility that the loci are linked. LOD scores ≧1 are particularly indicative of linkage. Classification of linkage has been proposed [see, e.g., Lander and Kruglyak (1995) Nature Genet. 11:241-2471 based on the number of times it would be expected to see a result at random in a dense, complete genome scan for linkage. Under such a classification scheme, suggestive linkage is statistical evidence that would be expected to occur one time at random in a genome scan, significant linkage is statistical evidence expected to occur 0.05 times in a genome scan (that is with probability 5%), highly significant linkage is statistical evidence expected to occur 0.001 times in a genome scan and confirmed linkage is significant linkage from one or a combination of initial studies that has subsequently been confirmed in a further sample. In the case of sibling pair-based linkage analysis, for example, suggestive, significant and highly significant linkage may correspond to LOD scores of 2.2, 3.6, and 5.4, respectively.

The relationship between two linked loci may also be defined in terms of the physical or genetic distance between the loci. Thus, two loci may be referred to as linked when they are located relatively close together on the same chromosome. For example, in the case of a polymorphic or marker site on a chromosome linked with a DNA segment associated with a disease or disorder, the marker may be located a particular number of base pairs (bp) or centiMorgans (cM) from the DNA segment. The particular distance, in bp or cM, between two linked loci can vary, but is small enough so that the linkage score, e.g., the LOD score, obtained in linkage analysis of the two loci (e.g., a marker and a trait such as a disease) is at least indicative of linkage (i.e., the loci are “relatively close” to each other) if not at least suggestive, significant or even highly significant linkage. A linked marker may be within the DNA segment associated with a trait (e.g., AD) and, further, may be a causative polymorphism in a disease (e.g., AD) gene, such as, for example, a polymorphism in a disease gene that is responsible for a defect in the disease gene. When the marker is located within a disease gene, it is referred to as coincident with the gene.

As used herein, a “disease or disorder DNA segment” is a gene or other DNA segment that either directly causes a disease or disorder or confers an increased or decreased susceptibility to a disease or disorder. Thus, for example, an Alzheimer's disease (AD) DNA segment is a gene or other DNA segment that either directly causes AD or confers an increased or decreased susceptibility to AD. A gene or DNA segment which causes a disease or disorder may, for example, have an allele that contains an alteration, e.g., a mutation, relative to another allele(s) of the gene or DNA segment, wherein the alteration can cause or give rise to a defect involved in the manifestation of a disease or disorder phenotype.

A gene or DNA segment that confers increased susceptibility to a disease or disorder may have an allele that predisposes an individual to the disease or disorder but is not an invariant cause of the disease or disorder. Thus, an allele that confers increased susceptibility to disease or disorder can increase the likelihood of developing the disease or disorder but is not sufficient alone to cause the disease or disorder. Such an allele may be referred to as a genetic risk factor for the disease or disorder and may be one of several genetic risk factors, which in turn may be one type of several types of risk factors. For example, other possible risk factors could include environmental risk factors. An allele of a gene or DNA segment that confers increased susceptibility to a disease or disorder can be over-represented in cases in case control studies and/or can be associated with affected individuals in a family-based association analysis.

A gene or DNA segment that confers decreased susceptibility to a disease or disorder can be under-represented in cases in case control studies and/or can be associated with unaffected individuals in a family-based association analysis.

As used herein, a “DNA segment associated with a disease or disorder” refers to an allele that either is a disease or disorder gene or DNA segment or is in linkage disequilibrium with a disease or disorder gene or DNA segment. For example, an allele that is a disease or disorder risk factor or disease or disorder susceptibility locus may be in linkage disequilibrium with an allele of a disease gene or DNA segment and thus may be a DNA segment associated with a disease or disorder. In another example, a DNA segment associated with a disease or disorder may be in linkage disequilibrium with a protective allele of a disease gene that confers a decreased susceptibility to a disease or disorder. DNA segments associated with a disease or disorder include genes as well as intergenic regions of DNA.

As used herein, the term “protective” with reference to an allele refers to an allele that is indicative of a decreased risk relative to the general population for a genetic disease, e.g., AD. The decreased risk associated with a protective allele may be identified as under-representation of the allele in cases relative to controls, and/or as a significant association between the allele and unaffected members of a family that contains affected members. A protective allele may be a variant of a DNA segment, such as a gene, that has a risk factor or disease allele. A protective allele may be a variant that is “functional” in that it participates in counteracting a defect that occurs in a genetic disease, e.g., AD, or may confer apparent “protection” against a disease by not conferring risk for the disease.

As used herein, the term “penetrance” refers to the ratio between the number of trait positive carriers of a particular allele and the total number of carriers of the allele in the population. Thus, a highly penetrant gene or allele will have a greater penetrance ratio than a weakly or moderately penetrant gene. Penetrance may also be considered as the percent probability that a carrier of a particular allele will express the corresponding phenotype.

As used herein, “prevalence” refers to the percentage of trait positive individuals that carry a particular allele.

As used herein, the term “effect size” with reference to a disease gene refers to the degree to which mutations or polymorphisms in a DNA segment, e.g., a gene, confer susceptibility to the disease taking into account the magnitude of prevalence and penetrance of the polymorphism.

As used herein, a sequence of nucleotides encoding a uPA protein refers to any sequence of nucleotides that encodes a protein that has a biological activity or functional activity of uPA, such as protease activity. Such sequence of nucleotides can be cDNA or can include introns. In addition, uPA genes, which include a promoter region and optionally additional regulatory regions, are provided.

As used herein, a portion of a uPA gene refers to any segment of a uPA gene that, alone or in combination with one or more other segments of a uPA gene, provides for and/or influence a function of the gene and can be determined empirically. For example, a protein coding segment of a uPA gene functions to yield a mRNA that encodes the amino acid sequence of a uPA protein. Untranslated sequence regions (UTRs), 5′ and 3′ UTRs, can function to regulate gene transcription, e.g., patterns and levels of transcription [see, e.g., Smicun et al. (1998) Eur. J. Biochem. 251:704-715]. Segments in gene-promoters can also function to initiate and regulate transcription, e.g., polymerase binding sites. Segments in other regions of a gene, for example, sequences upstream of promoter elements, can also regulate transcription, such as enhancer, repressor and silencer elements. Introns can also contain elements that can influence gene function, such as transcript generation and splicing.

As used herein, the phrase “level of risk” with respect to a disease or disorder refers to an individual's risk of having or developing a disease or disorder. Level of risk can be based on an individual's genetic makeup or genotype, such as whether an individual possesses one or more particular alleles having one or more particular polymorphisms or variants at a particular polymorphic region(s). For example, an individual's level of risk for a disease can be high or increased if the individual possesses one or more particular alleles that may be associated with risk for the disease, or can be low or decreased if the individual possesses one or more particular alleles that may be associated with protection against the disease. An increased or decreased level of risk can be referred to as an “altered level of risk.”As used herein, the term “protective” with reference to an allele refers to an allele that is indicative of a decreased risk relative to the general population for a genetic disease, e.g., AD. The decreased risk associated with a protective allele may be identified as under-representation of the allele in cases relative to controls, and/or as a significant association between the allele and unaffected members of a family that contains affected members. A protective allele may be a variant of a DNA segment, such as a gene, that has a risk factor or disease allele. A protective allele may be a variant that is “functional” in that it participates in counteracting a defect that occurs in a genetic disease, e.g., AD, or may confer apparent “protection” against a disease by not conferring risk for the disease.

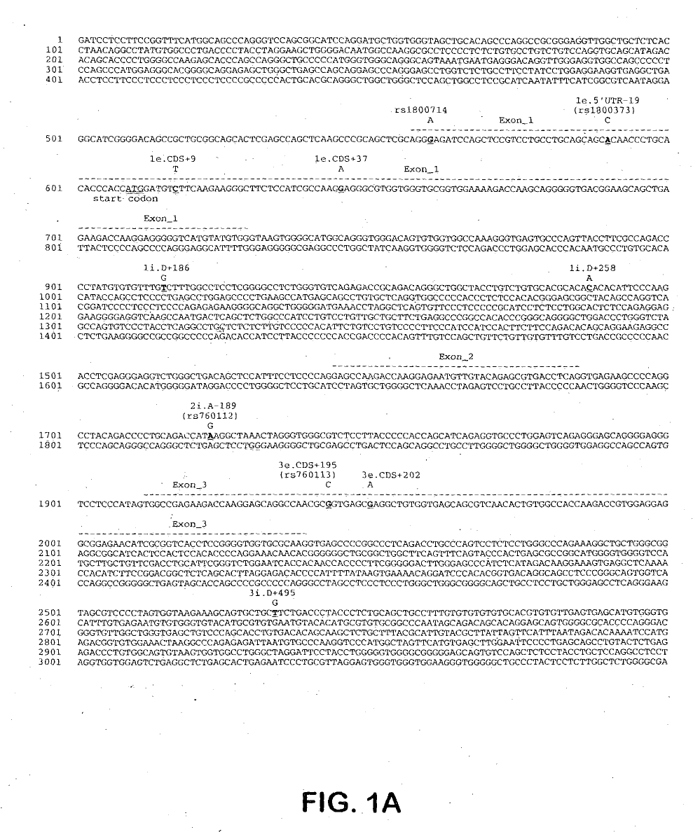

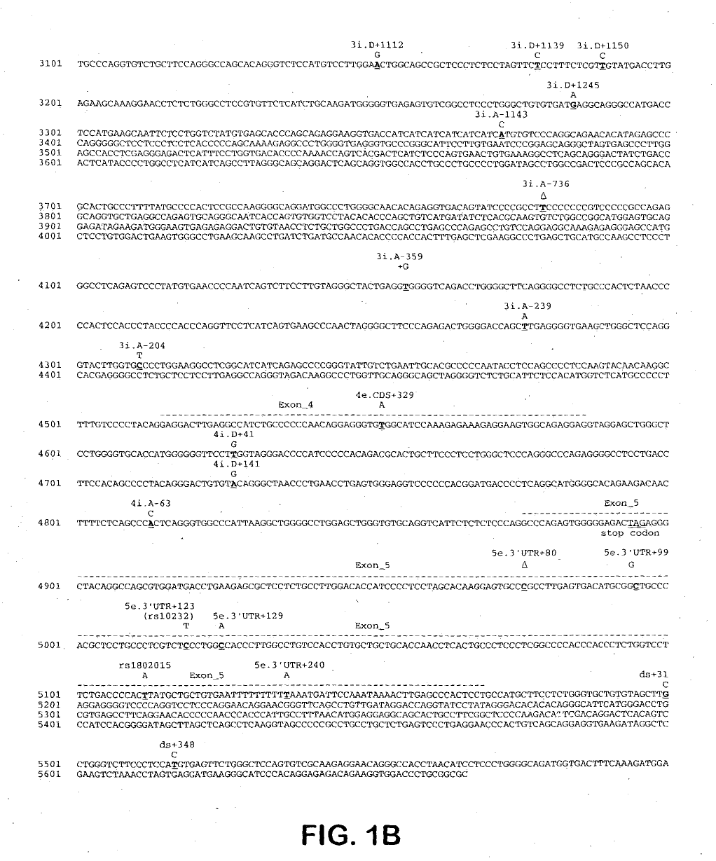

As used herein, “SNCG” refers to y-synuclein gene (a.k.a. persyn and breast cancer-specific gene 1 or BCSG1) such as the human y-synuclein gene set forth herein in FIG. 1 and in SEQ ID NO:72.

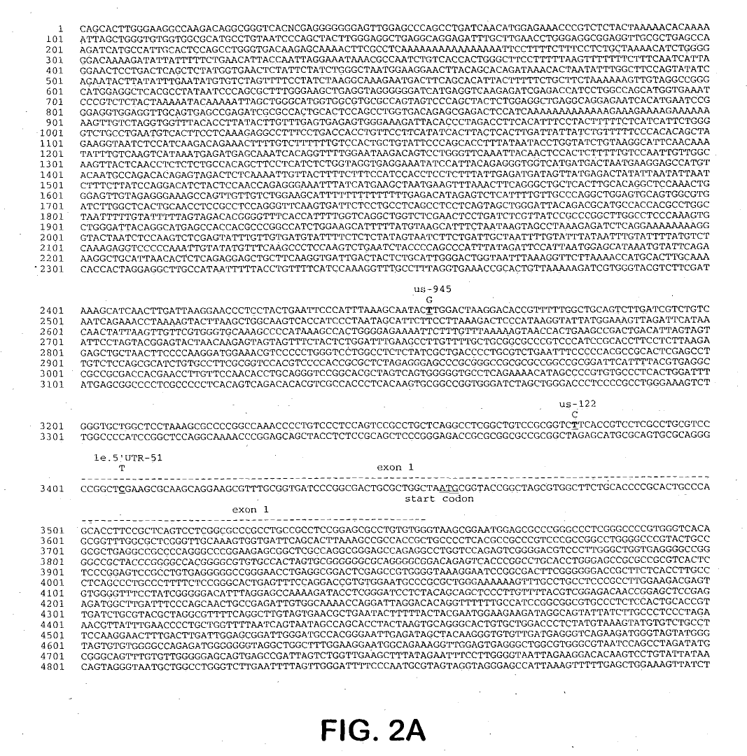

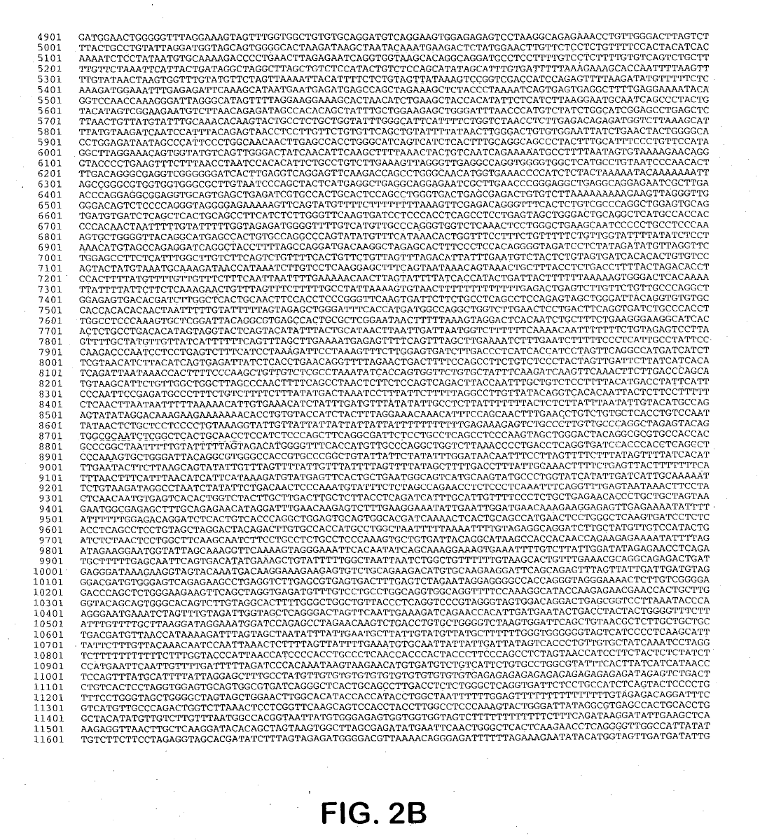

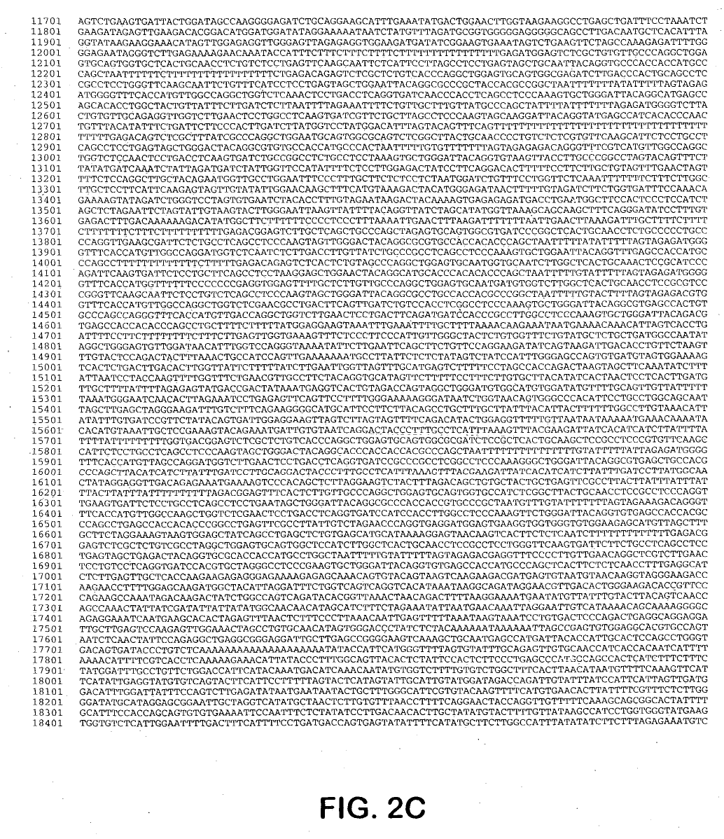

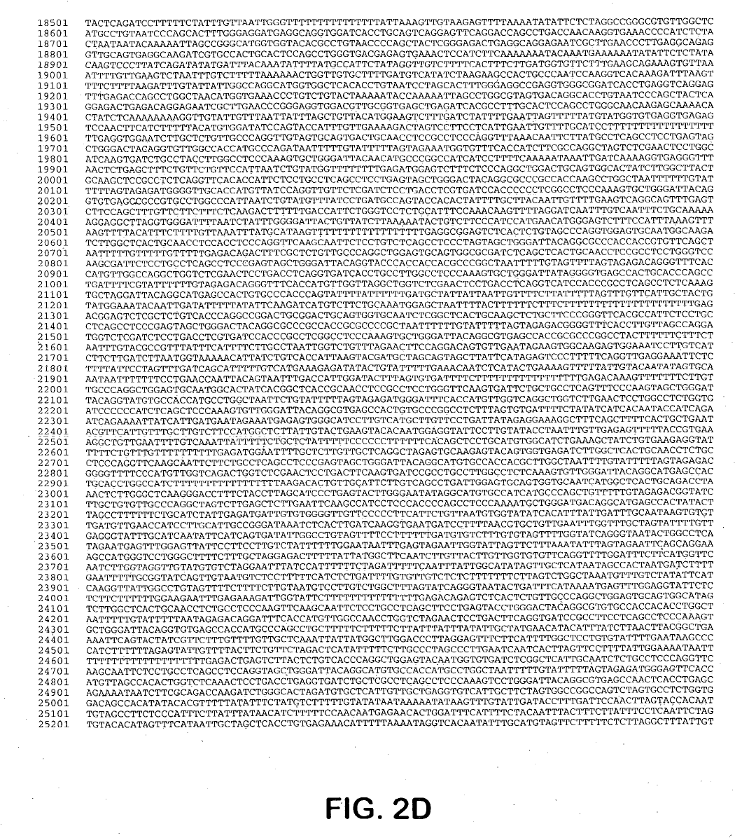

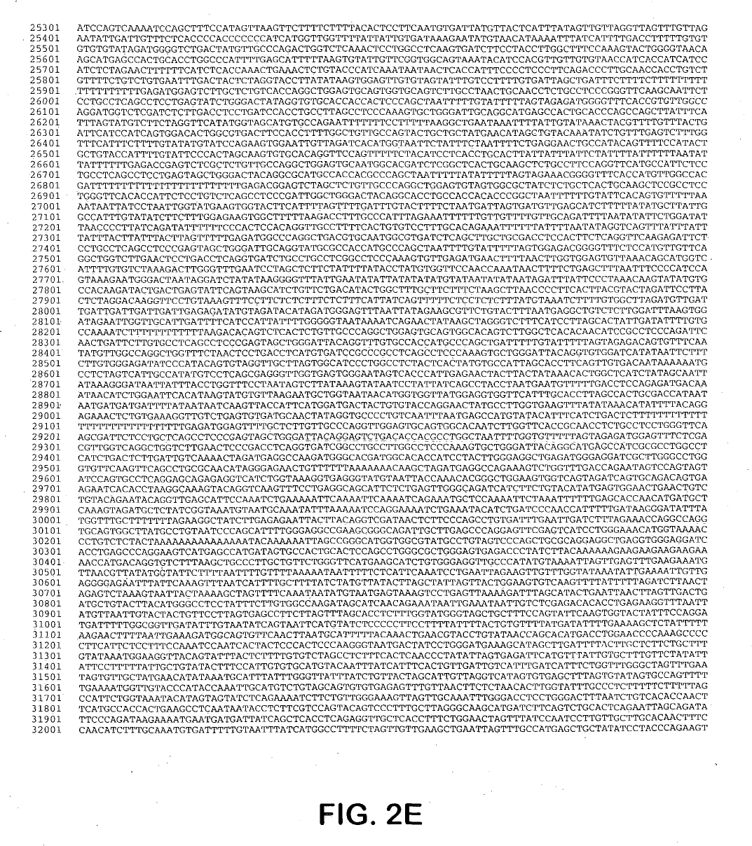

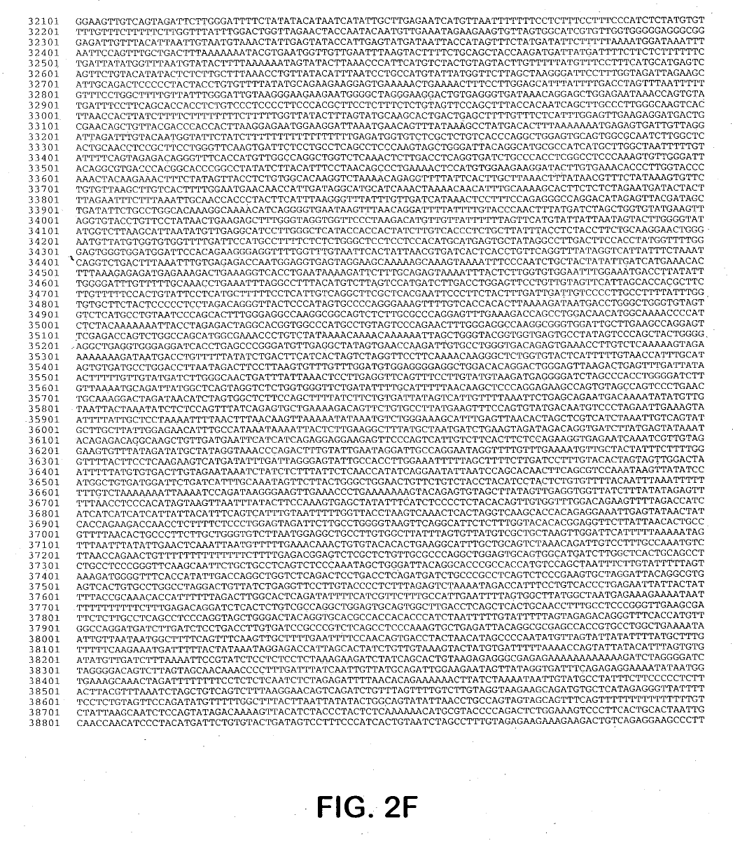

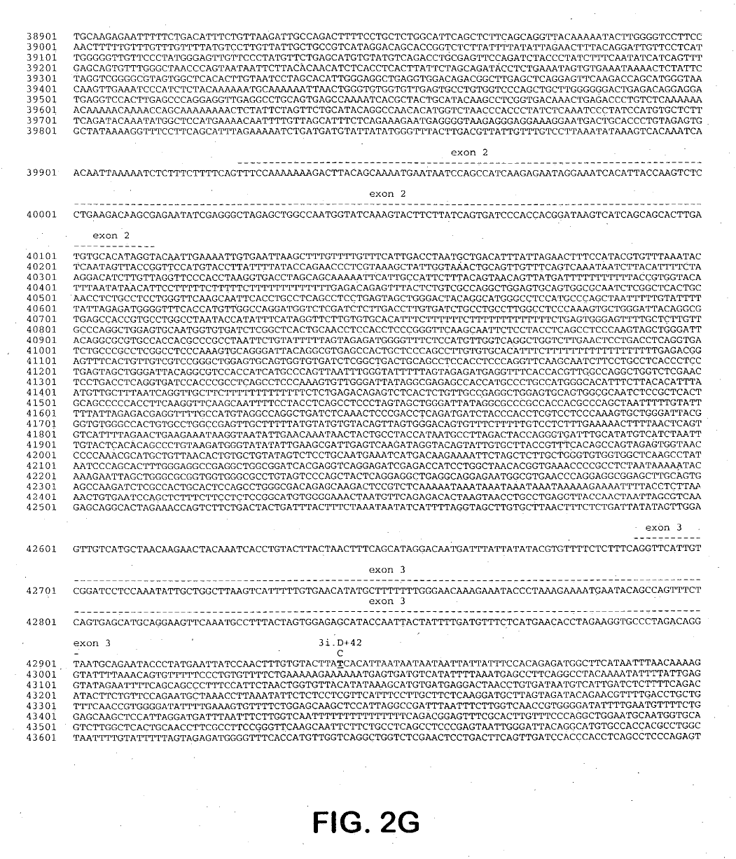

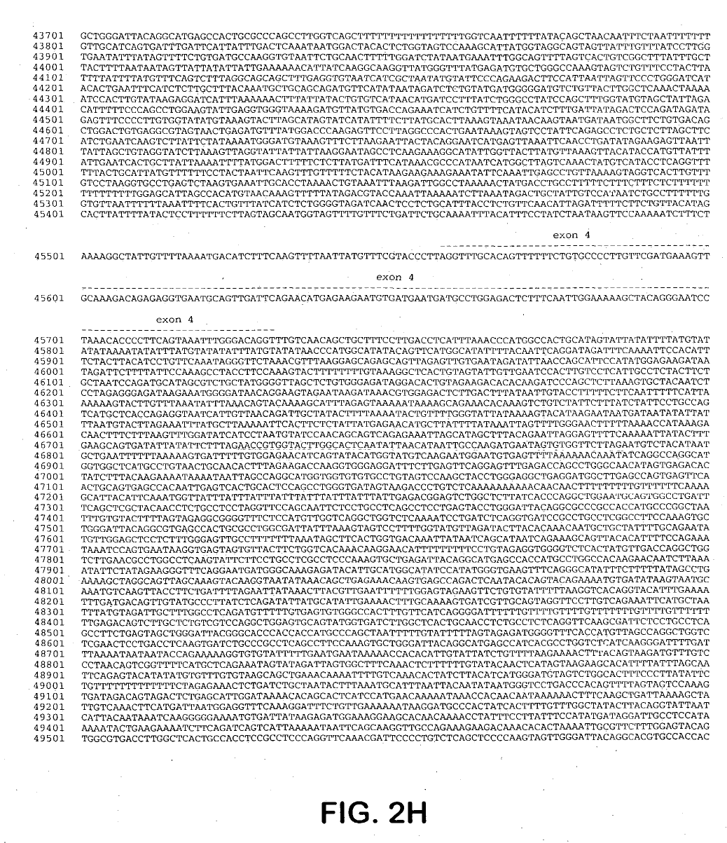

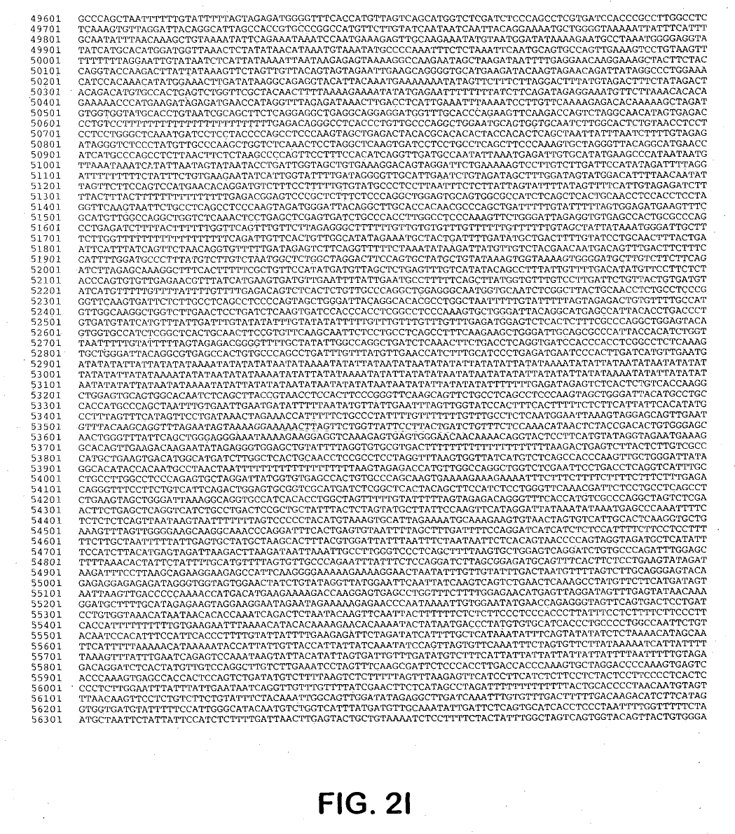

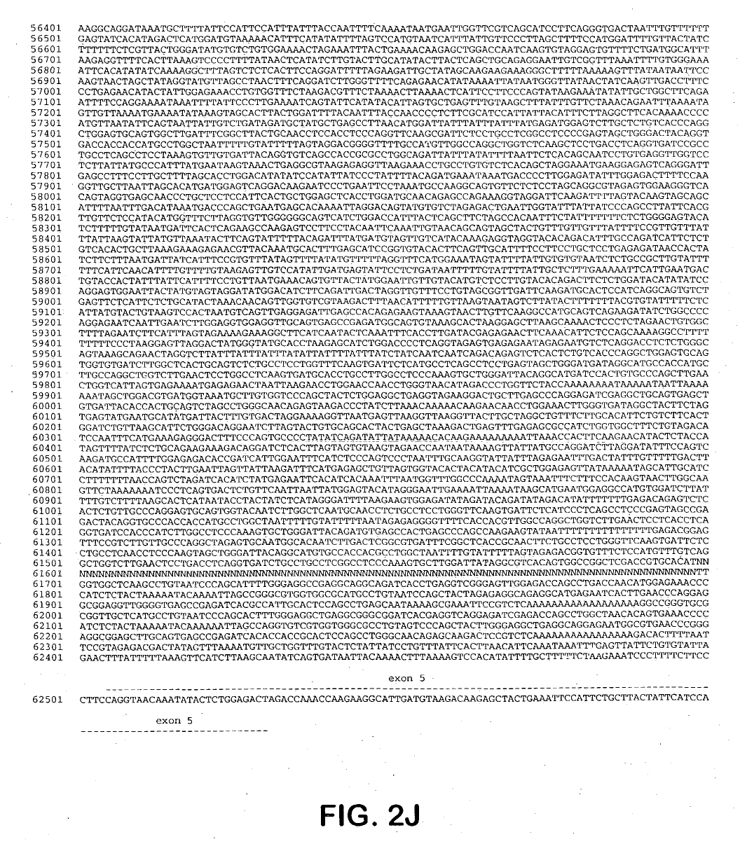

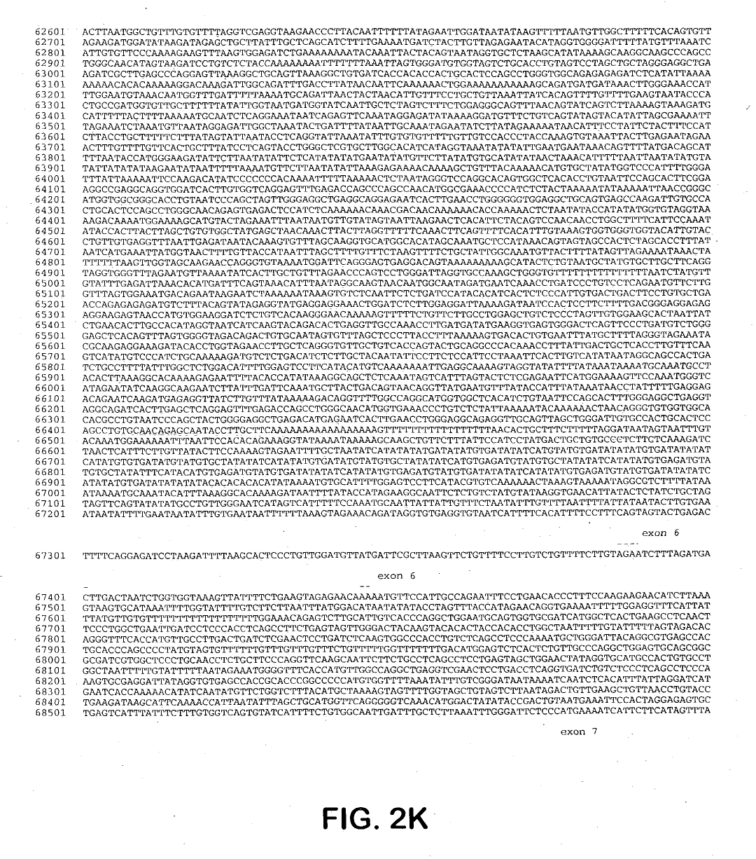

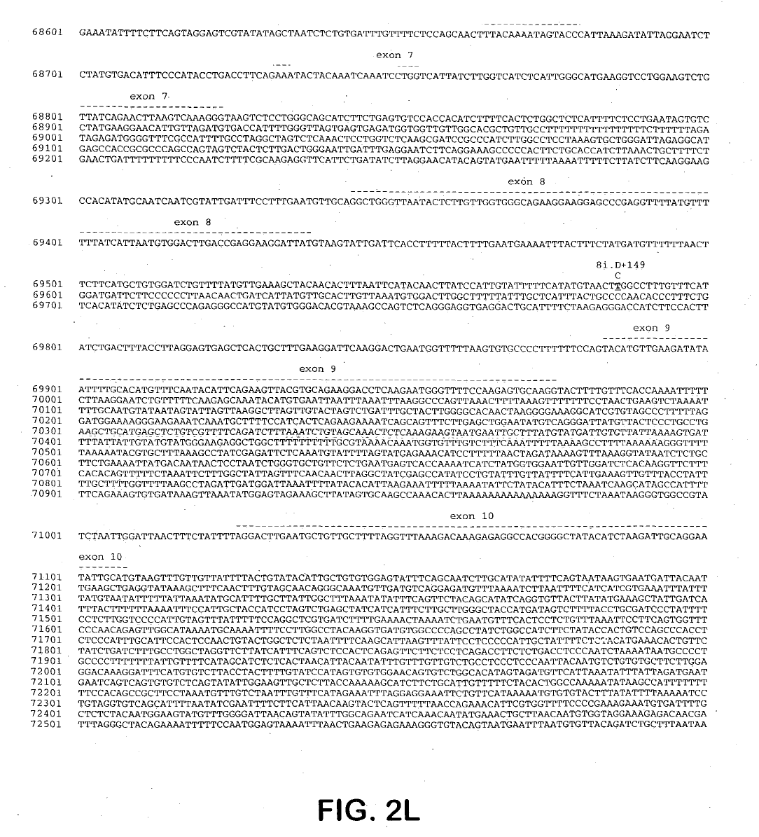

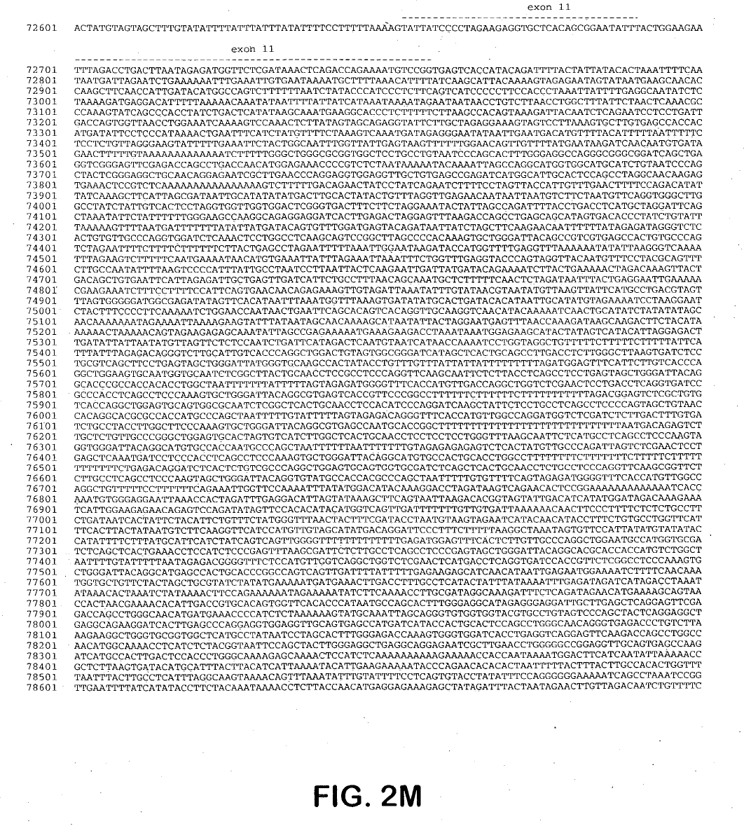

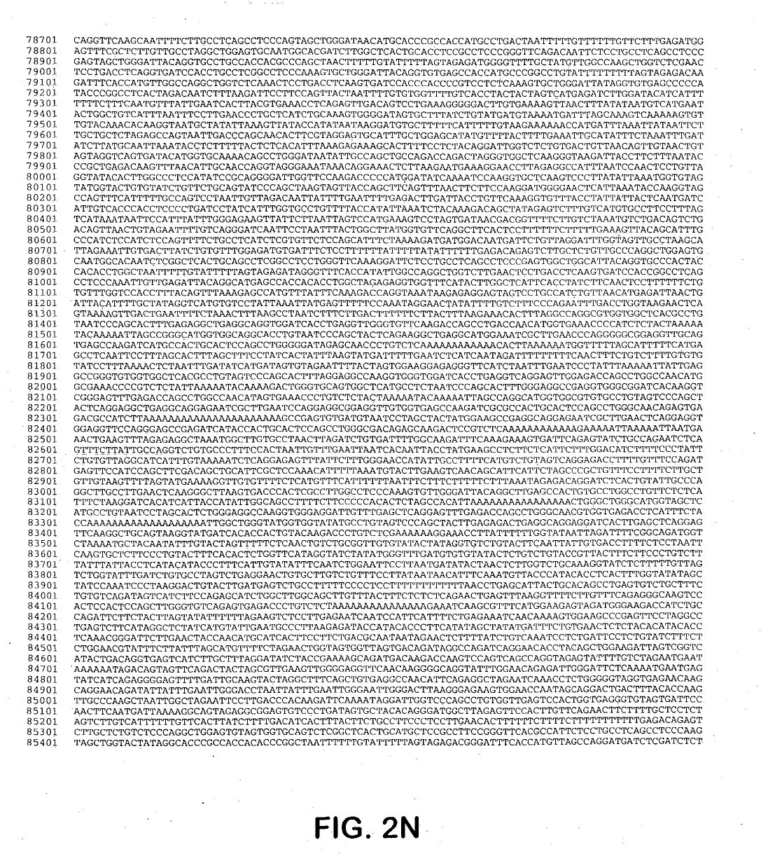

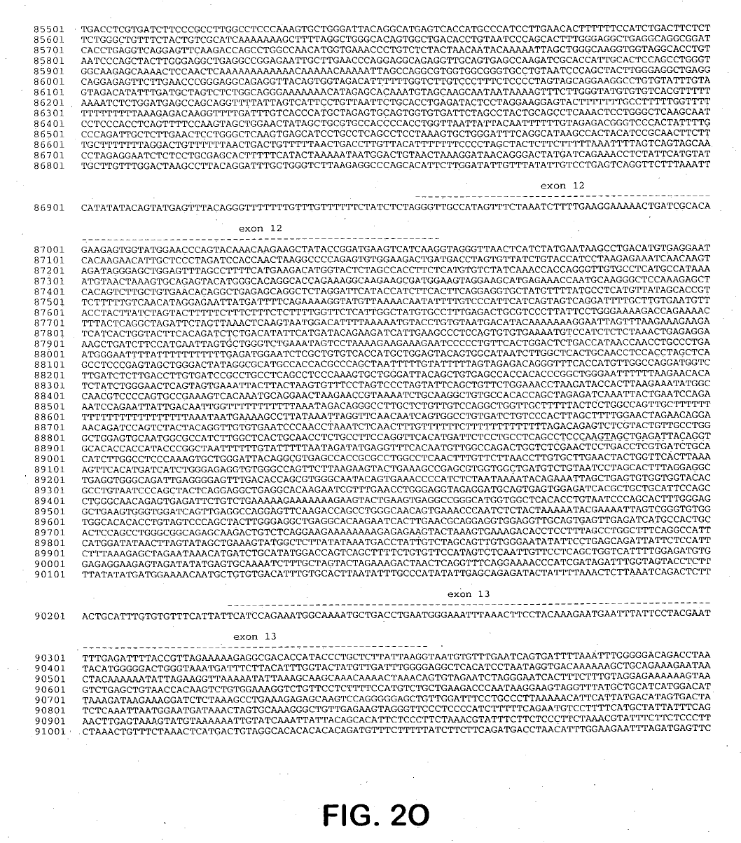

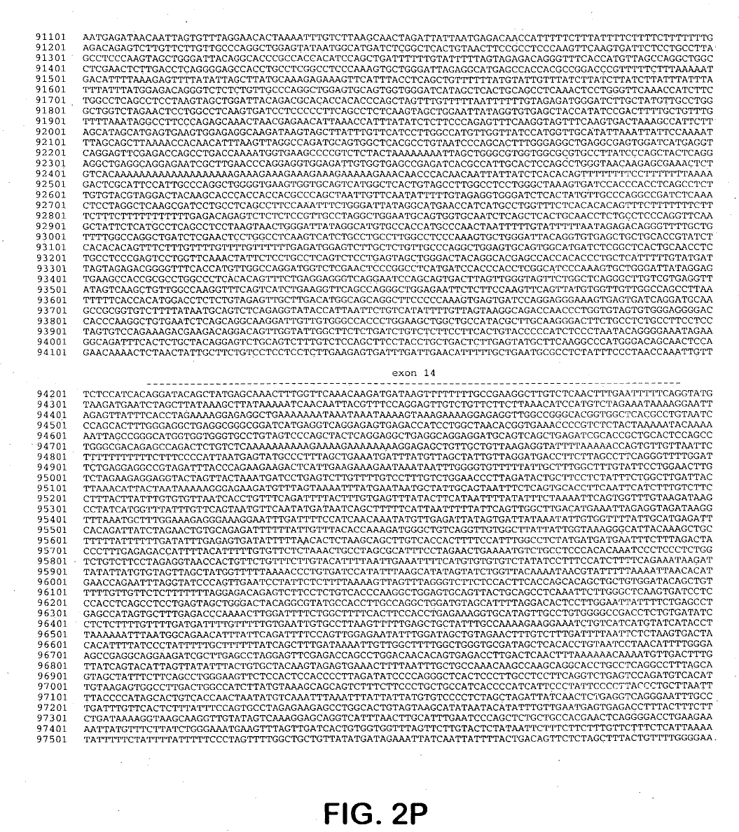

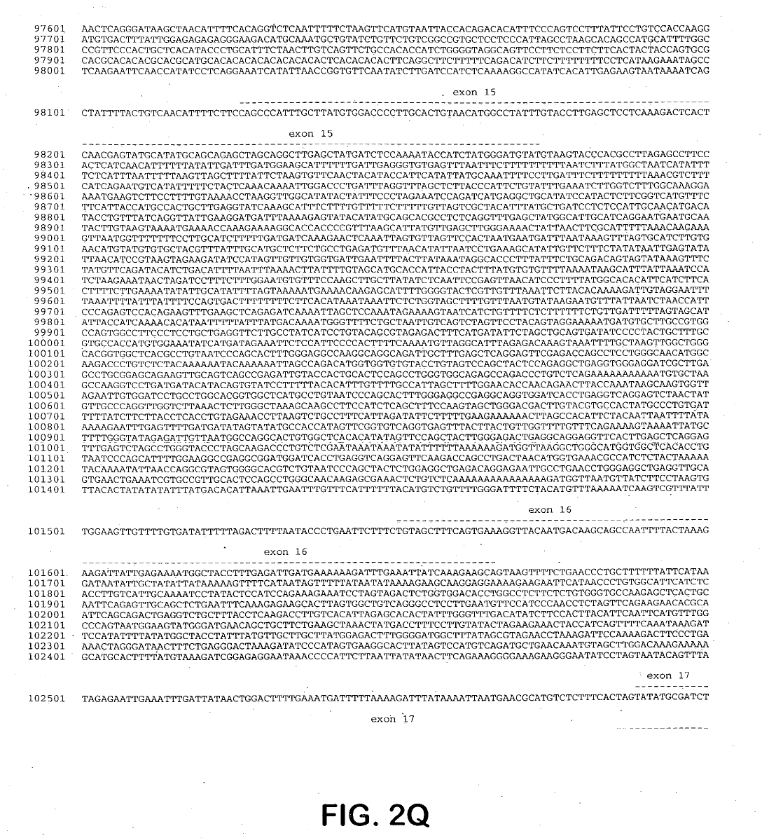

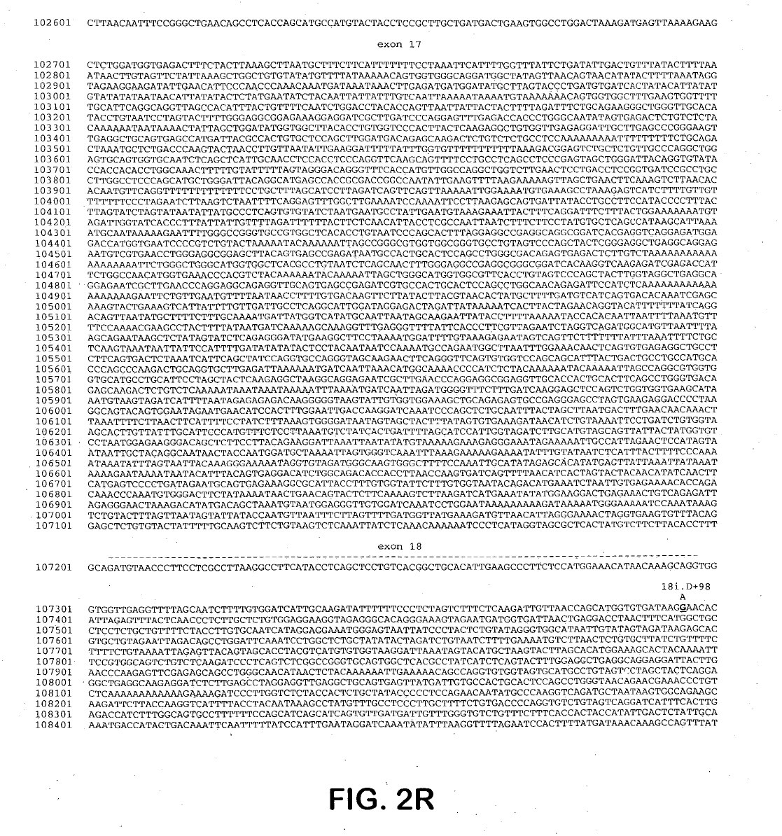

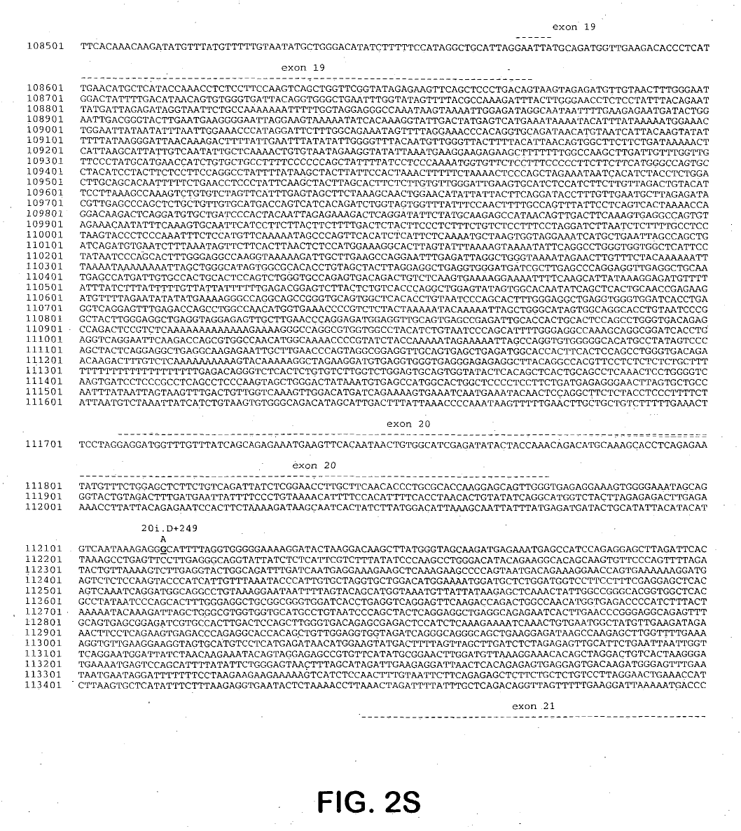

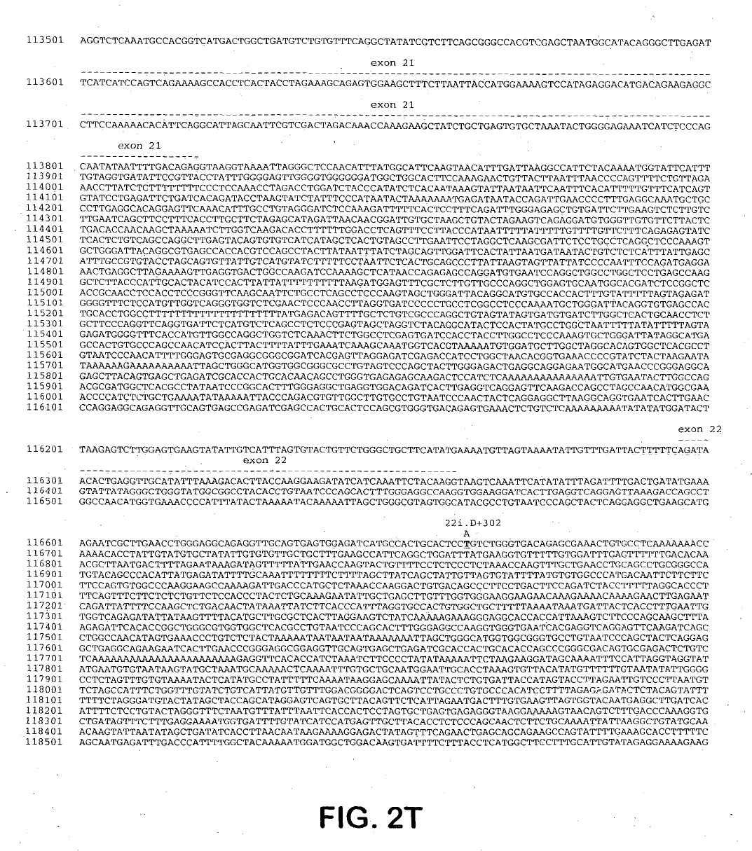

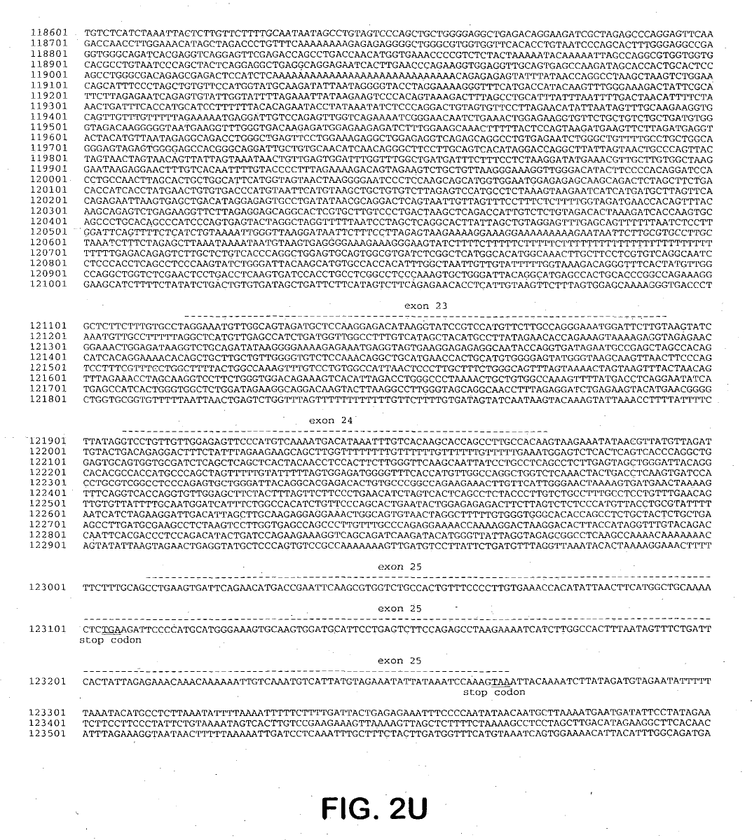

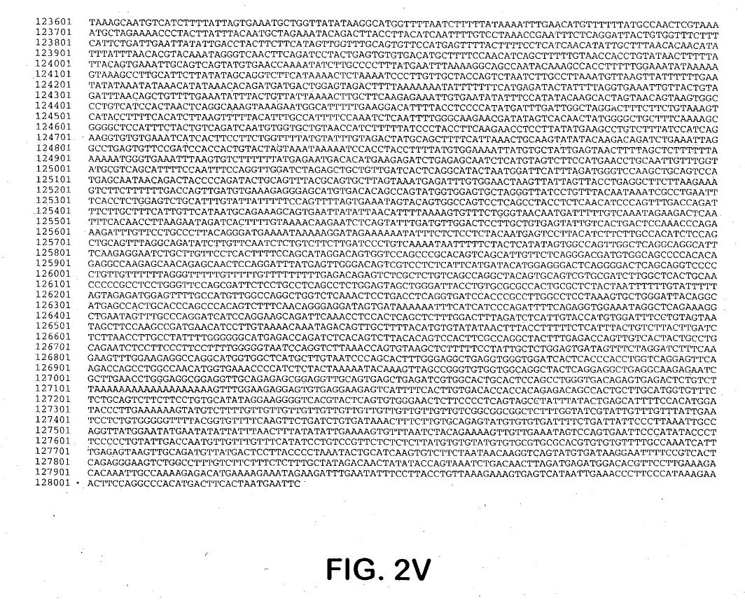

As used herein, “IDE” refers to Insulin Degrading Enzyme gene such as the human insulin-degrading enzyme gene set forth herein in FIG. 2 and in SEQ ID NO:186.

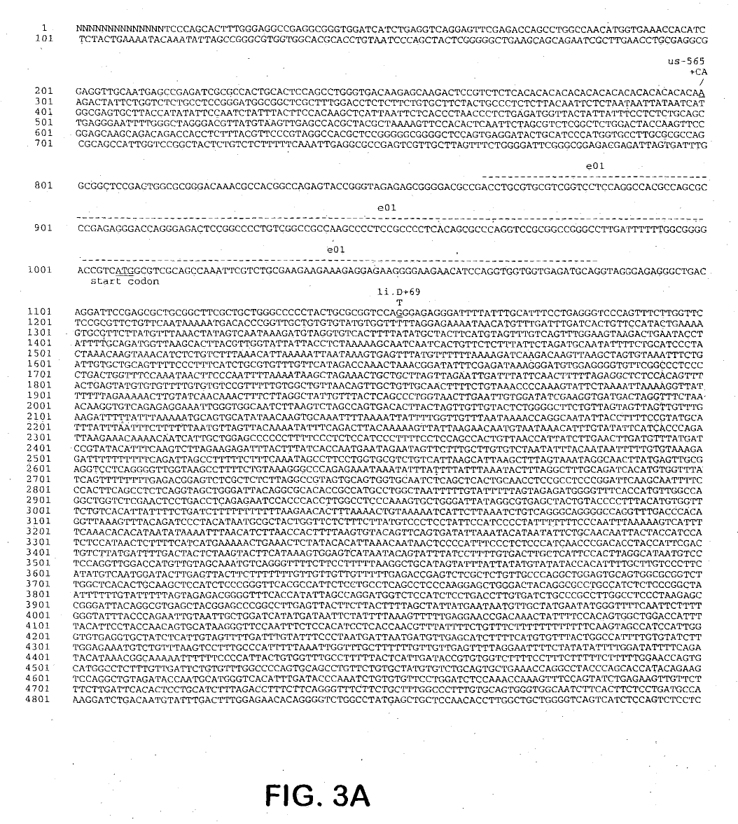

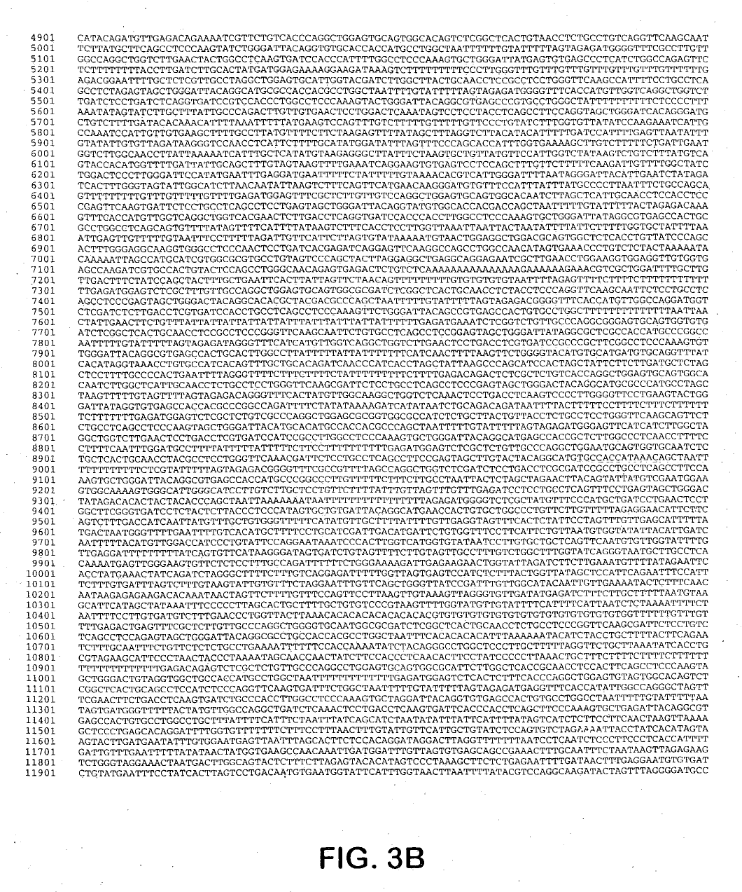

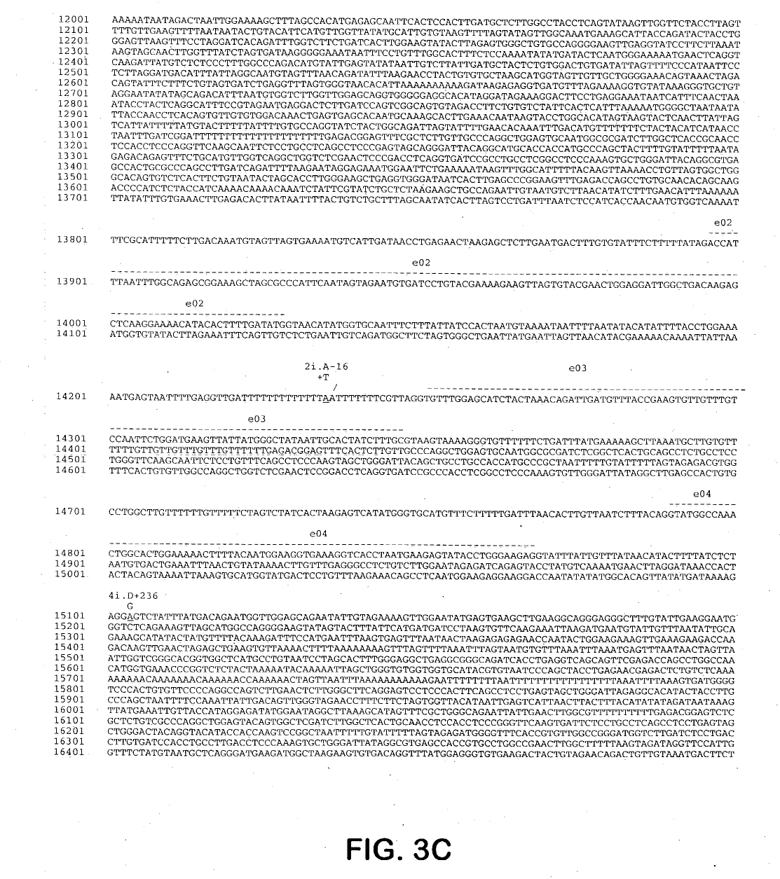

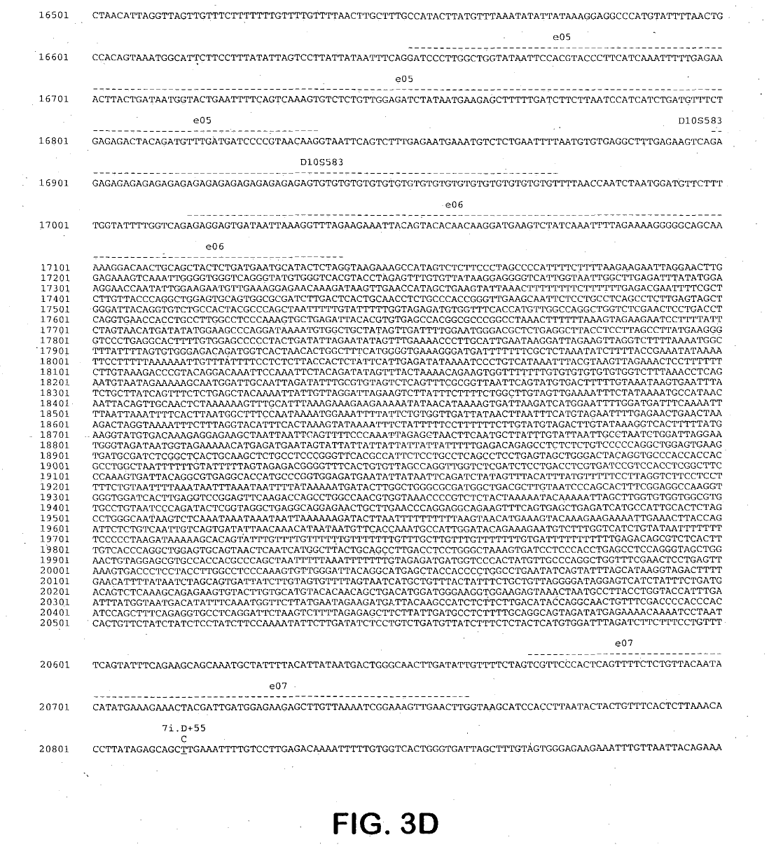

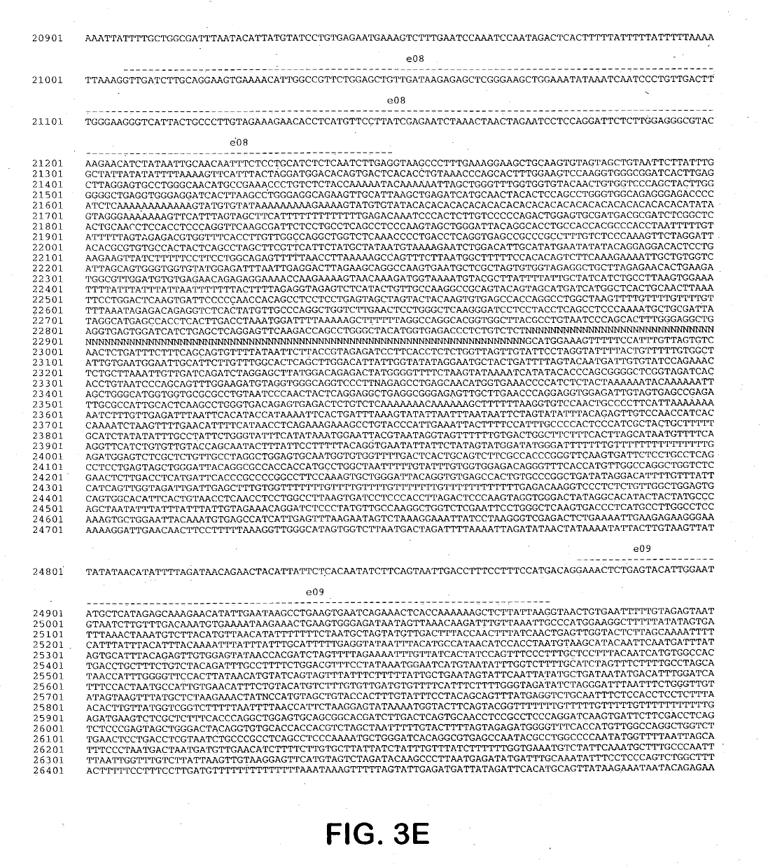

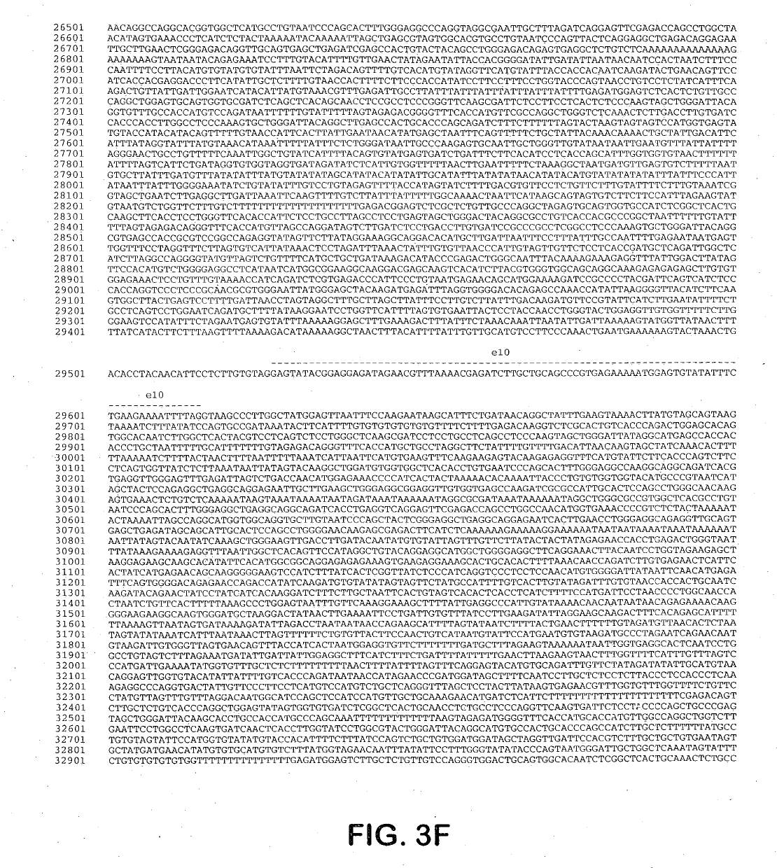

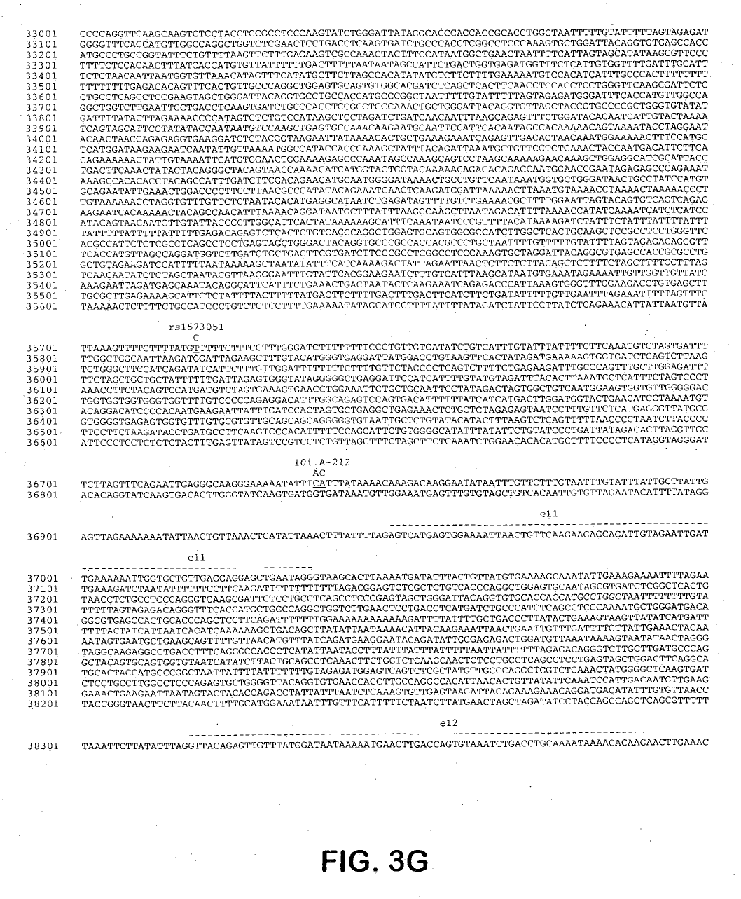

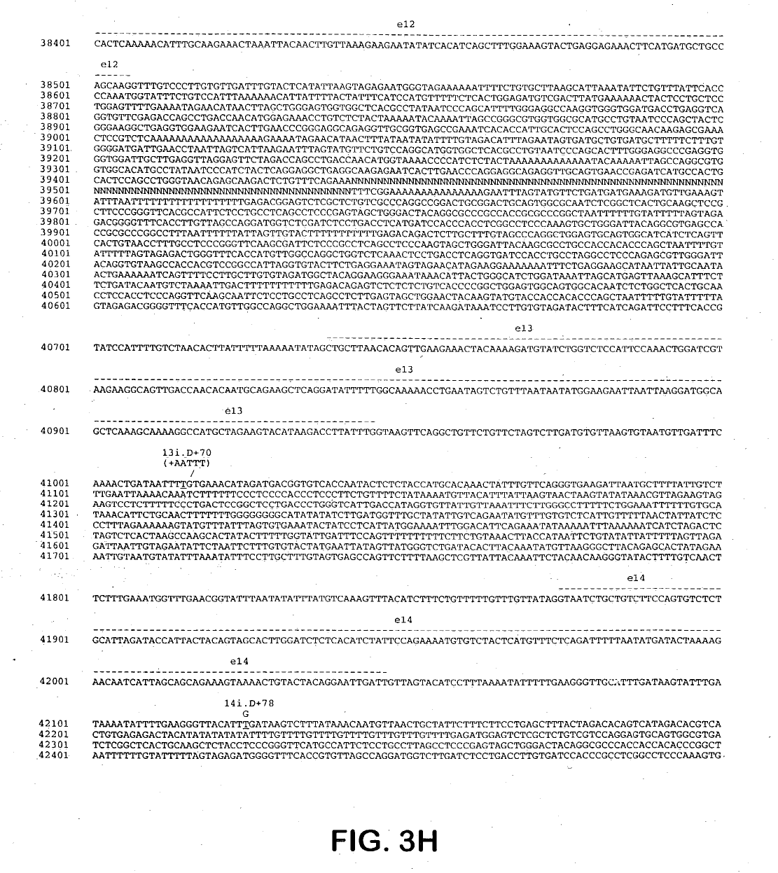

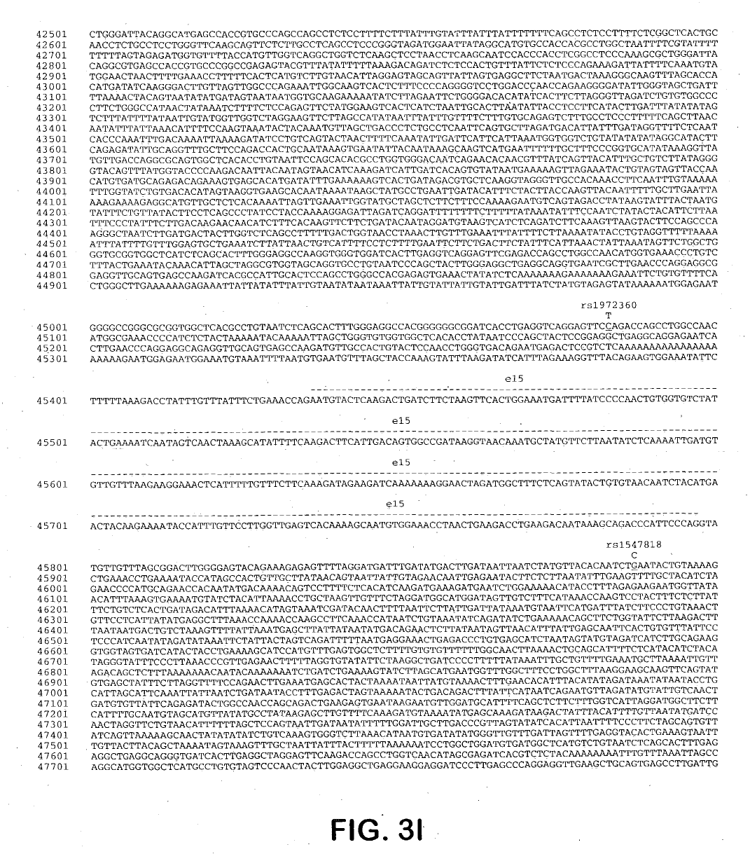

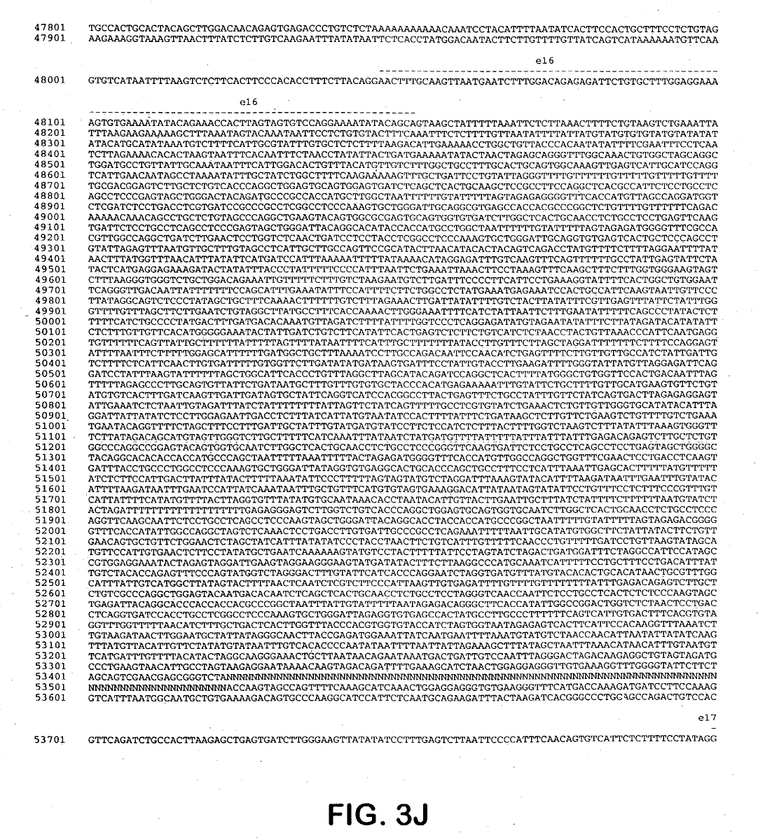

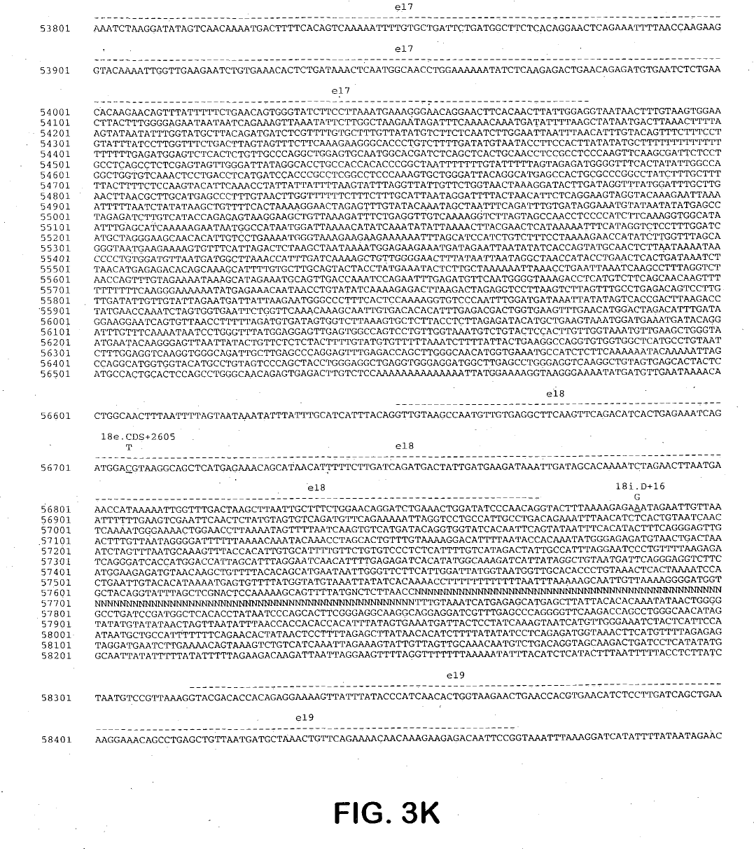

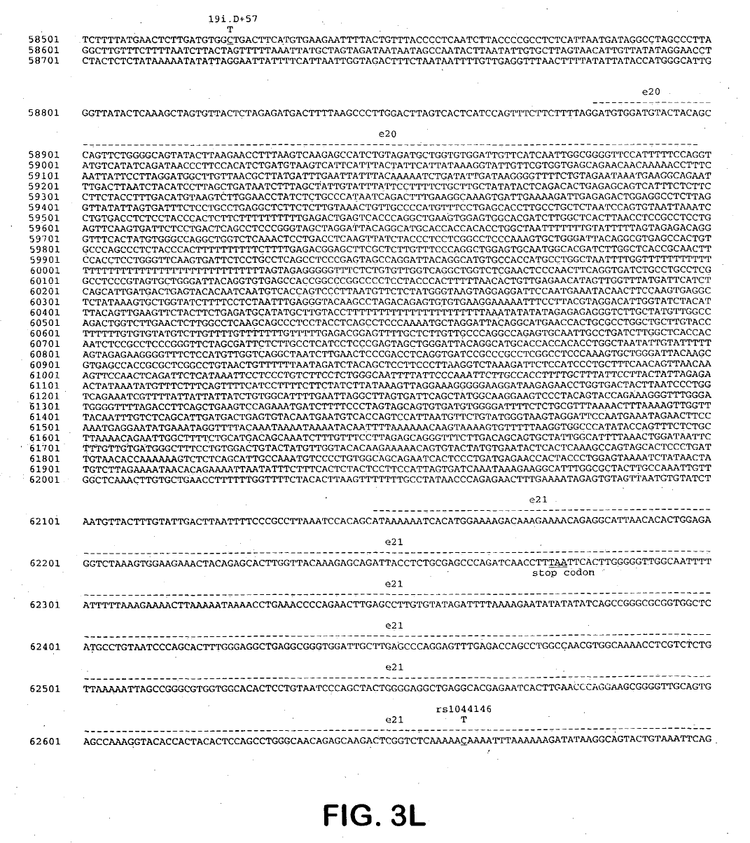

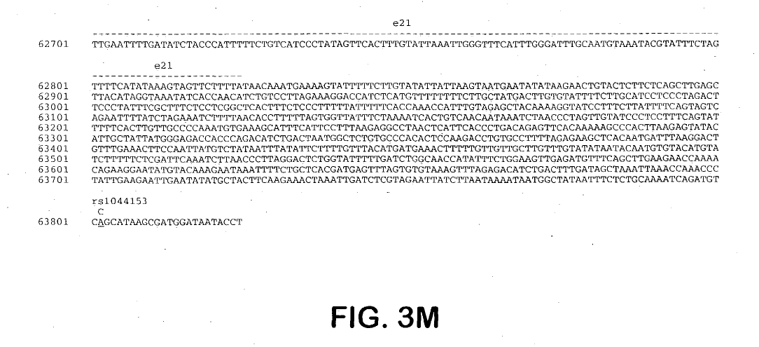

As used herein, “KNSL” refers to Kinesin I-like gene such as the human kinesin 1-like gene set forth herein in FIG. 3 and in SEQ ID NO:347.

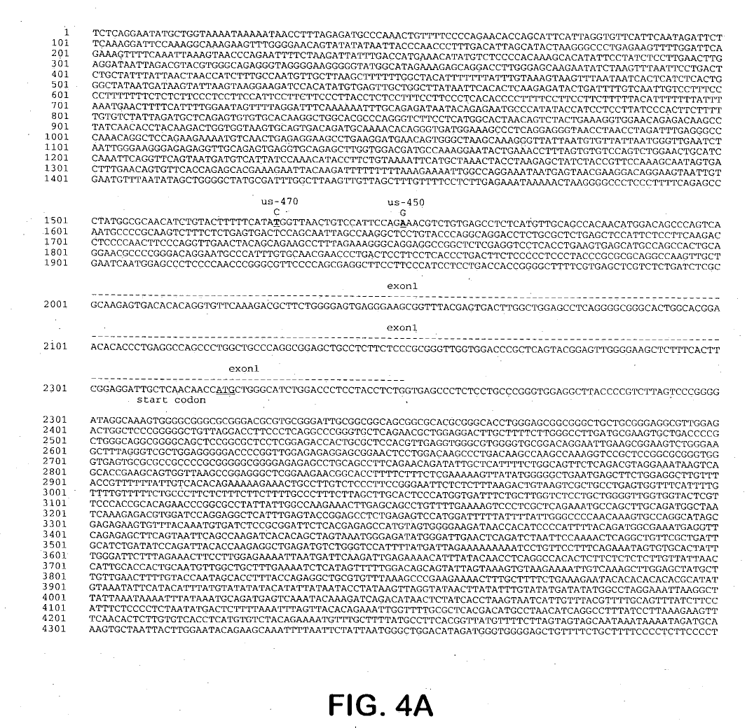

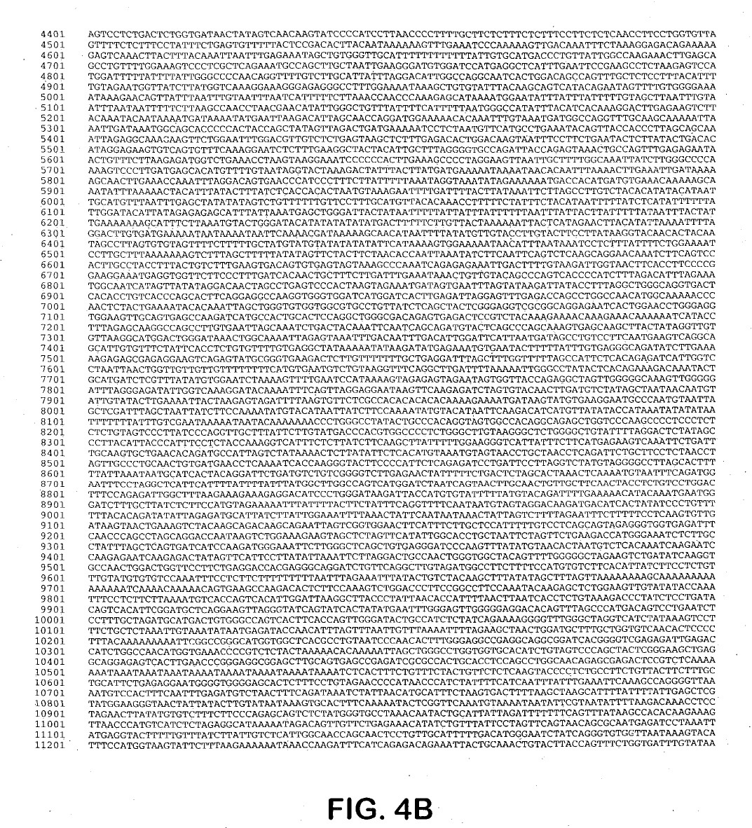

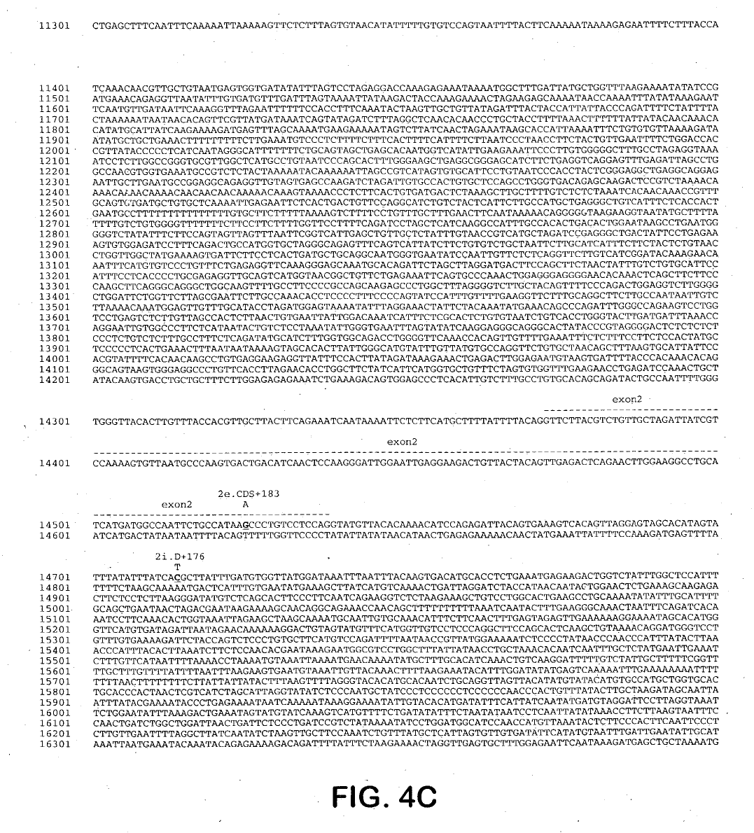

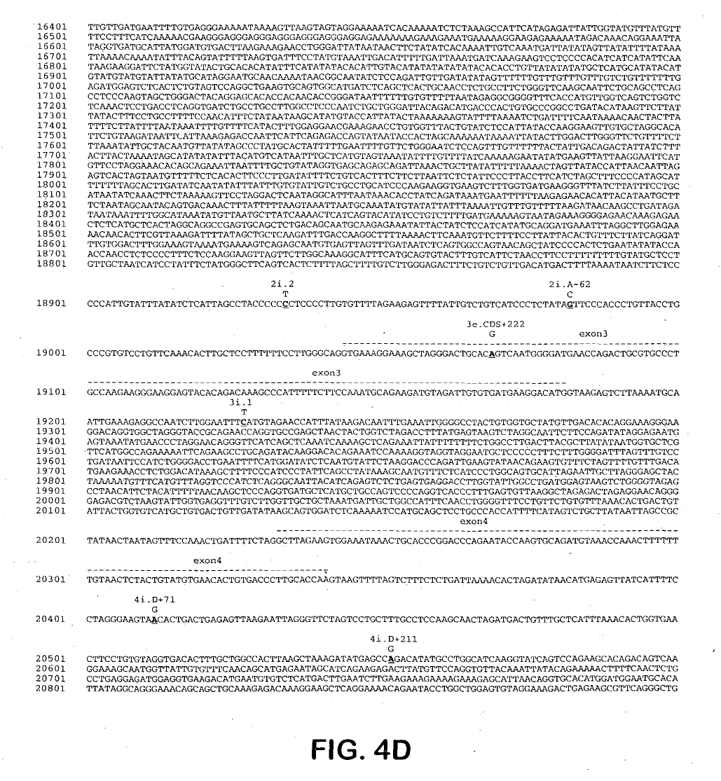

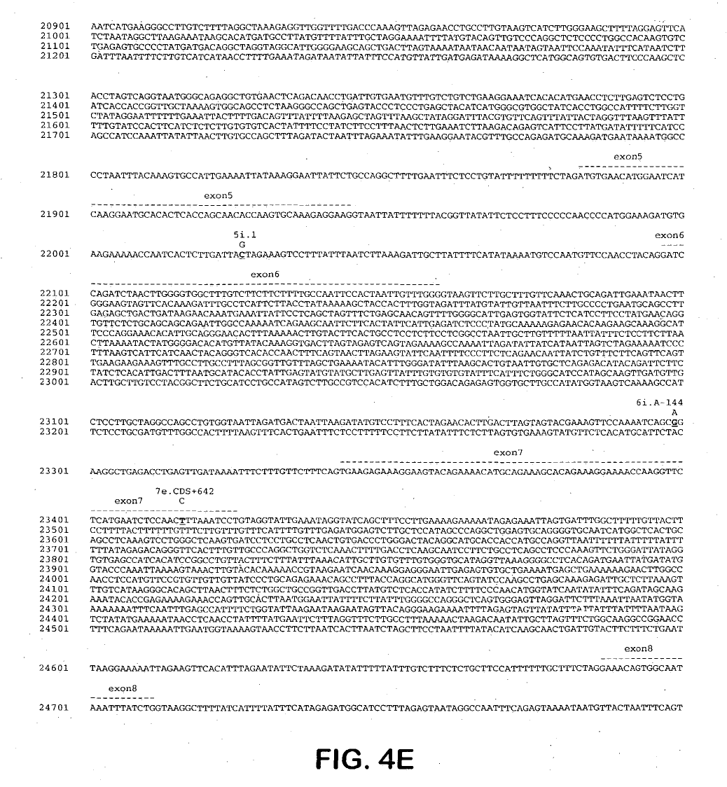

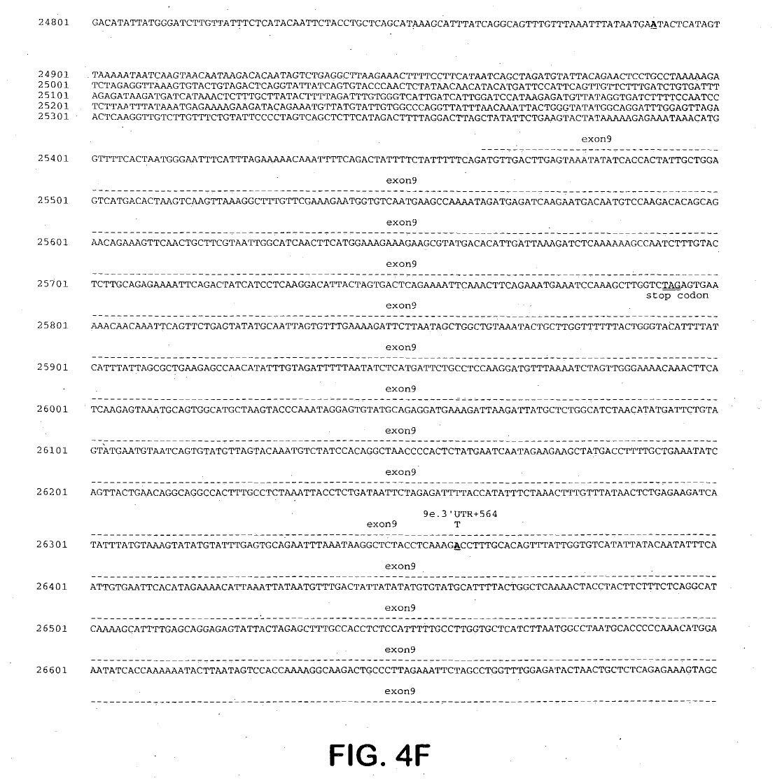

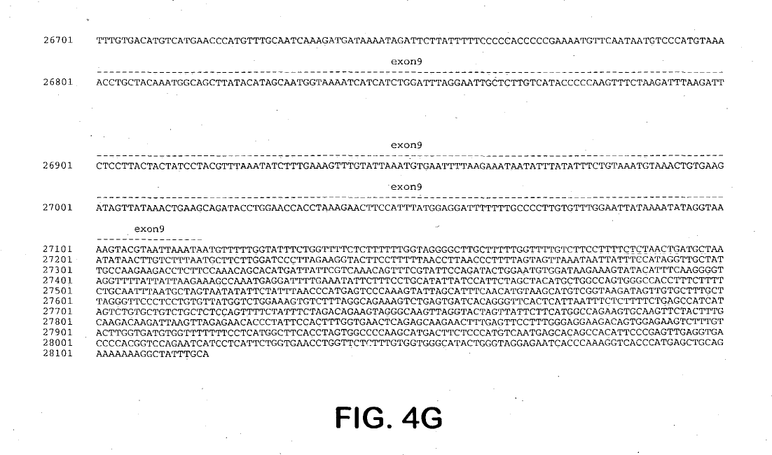

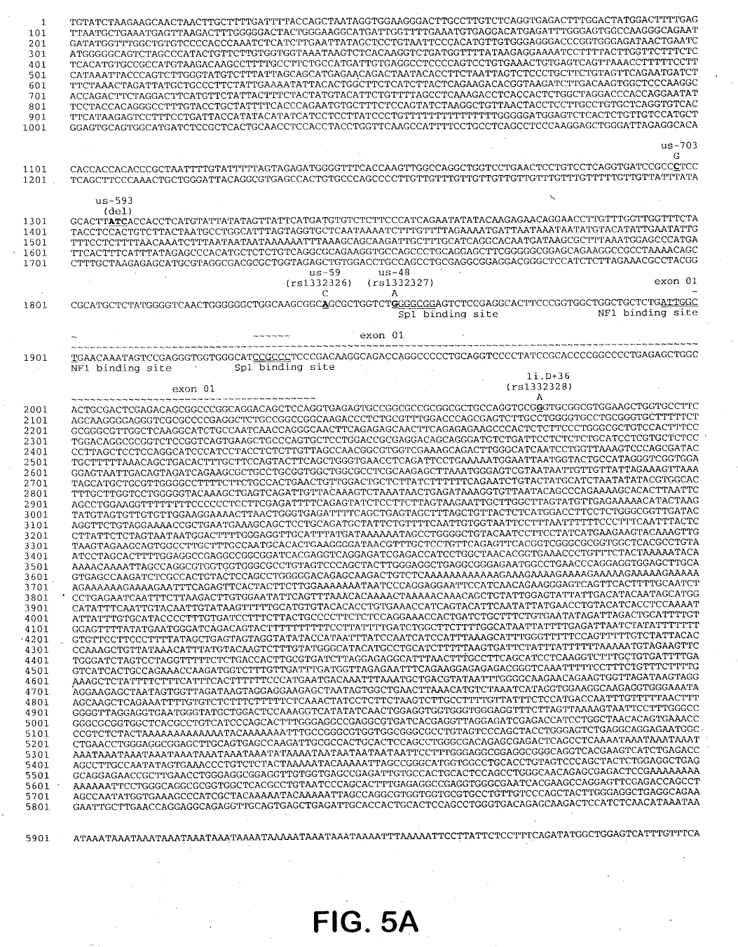

As used herein, “TNFRSF6” refers to a tumor necrosis factor receptor superfamily member 6 gene such as the human tumor necrosis factor receptor superfamily member 6 gene set forth herein in FIG. 4 and in SEQ ID NO:402.

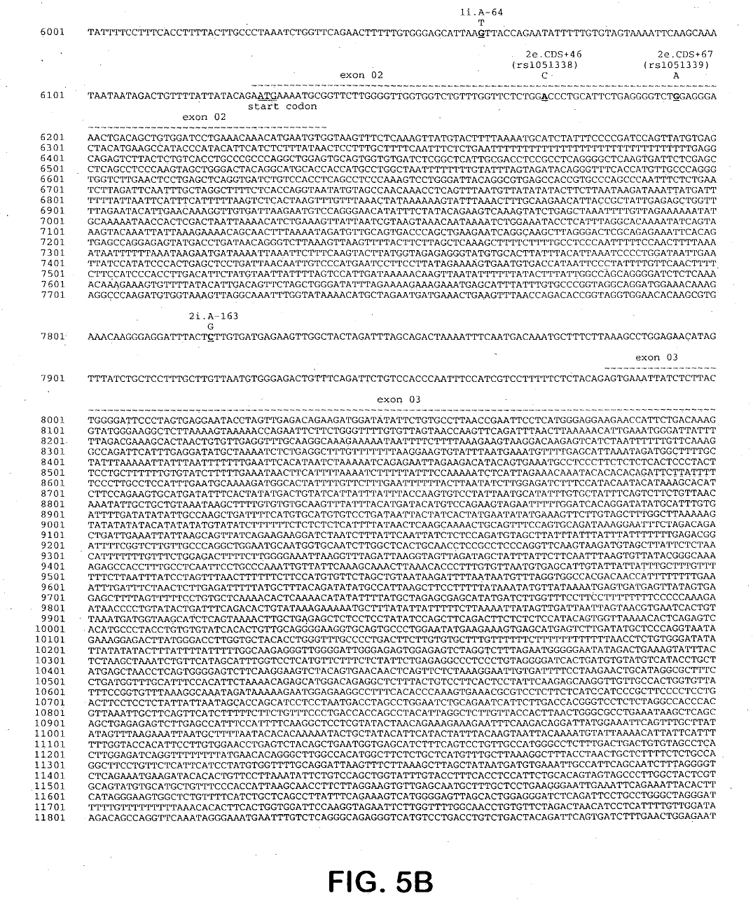

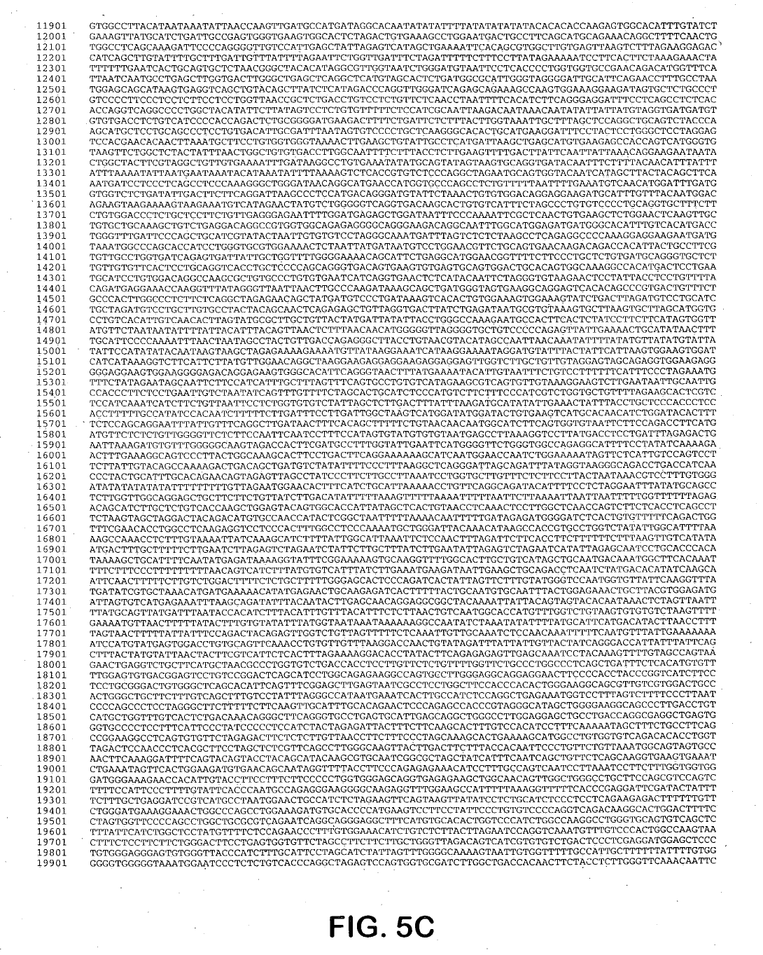

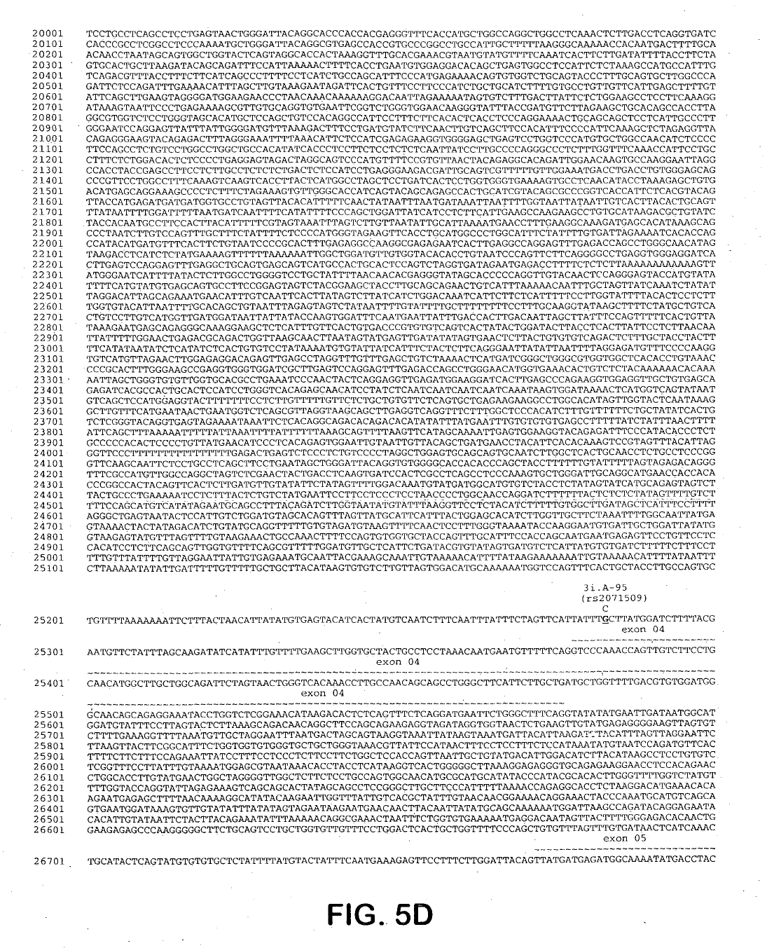

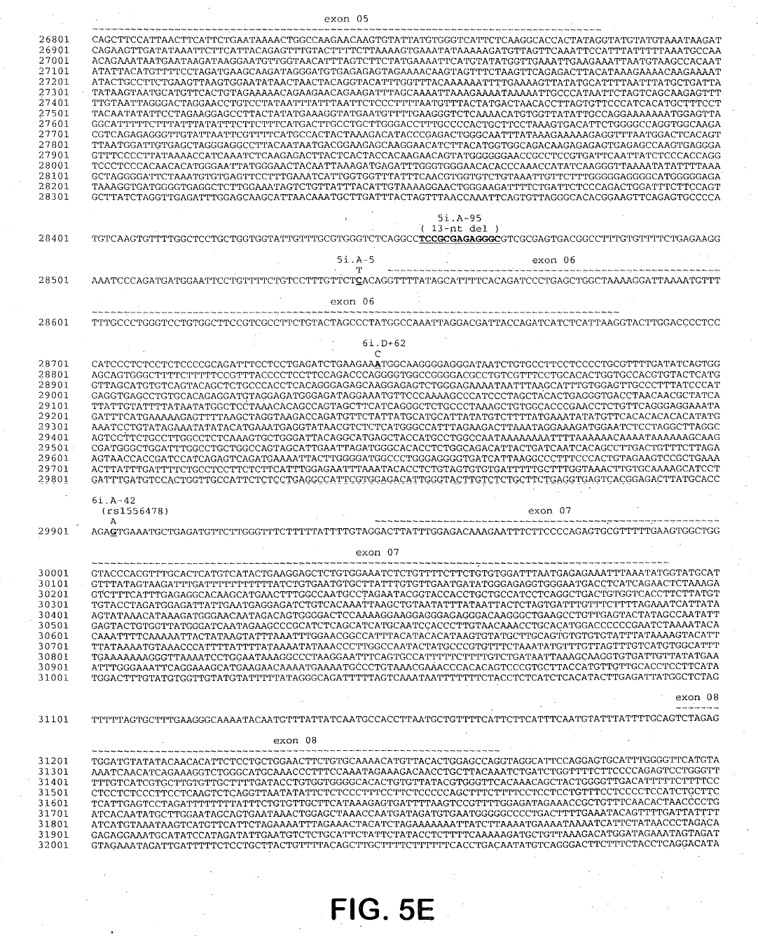

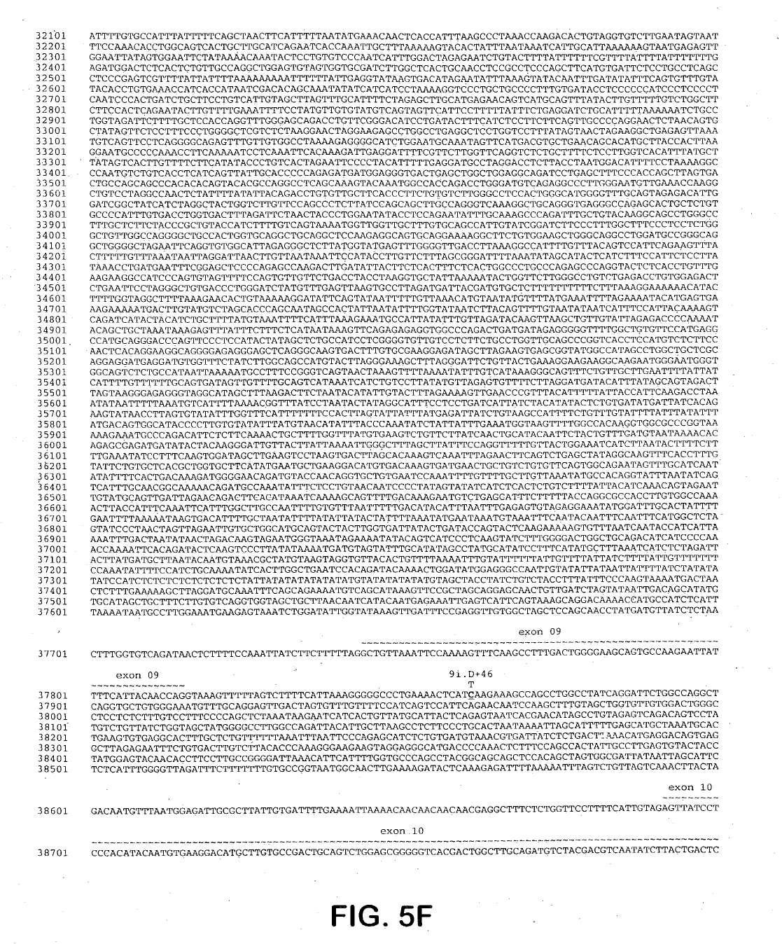

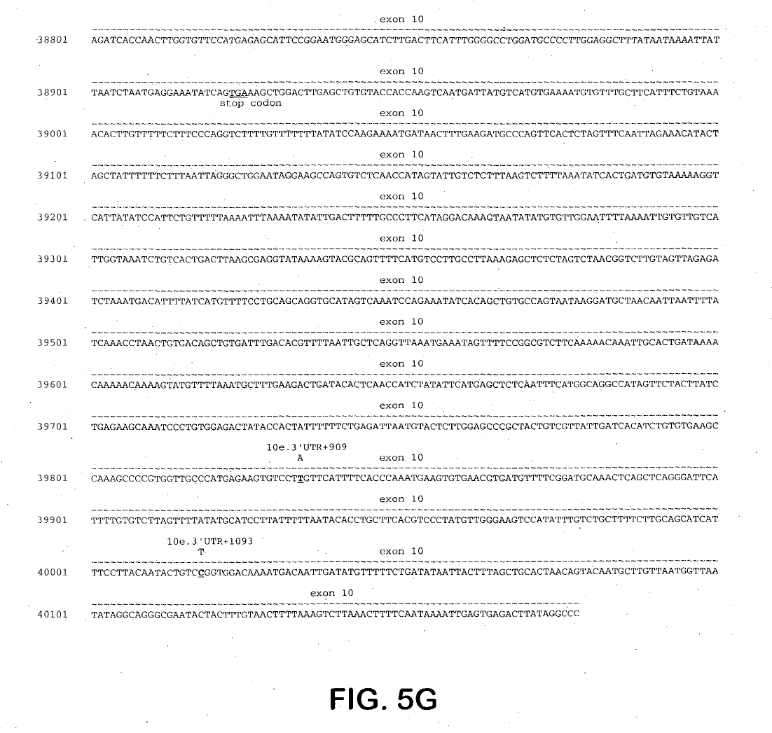

As used herein, “LIPA” refers to a lysosomal acid lipase (a.k.a. acid cholesteryl ester hydrolase and cholesterol ester hydrolase) gene such as the human lysosomal acid lipase gene set forth herein in FIG. 5 and in SEQ ID NO:467.

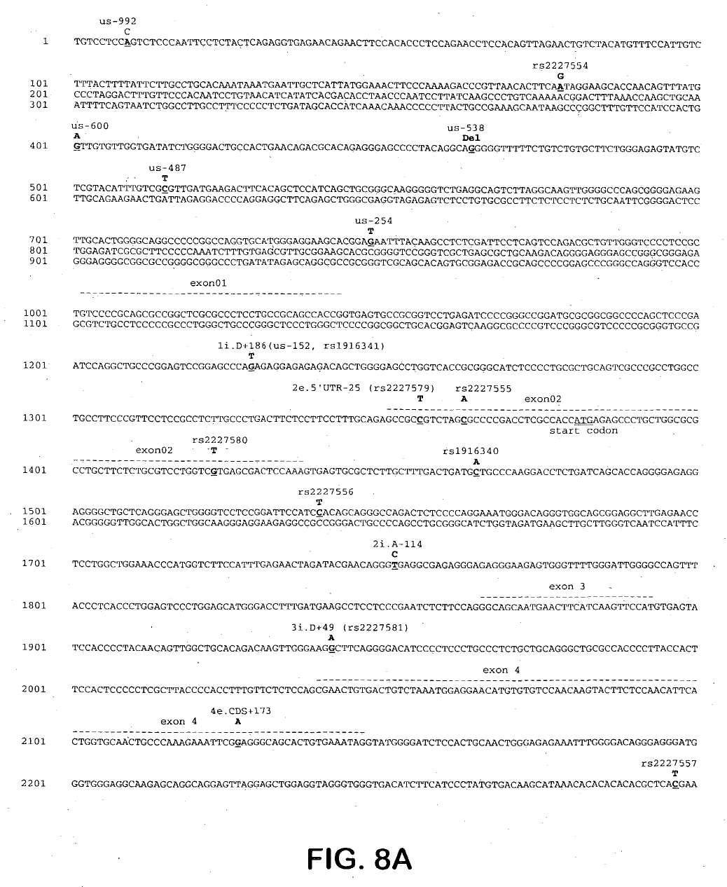

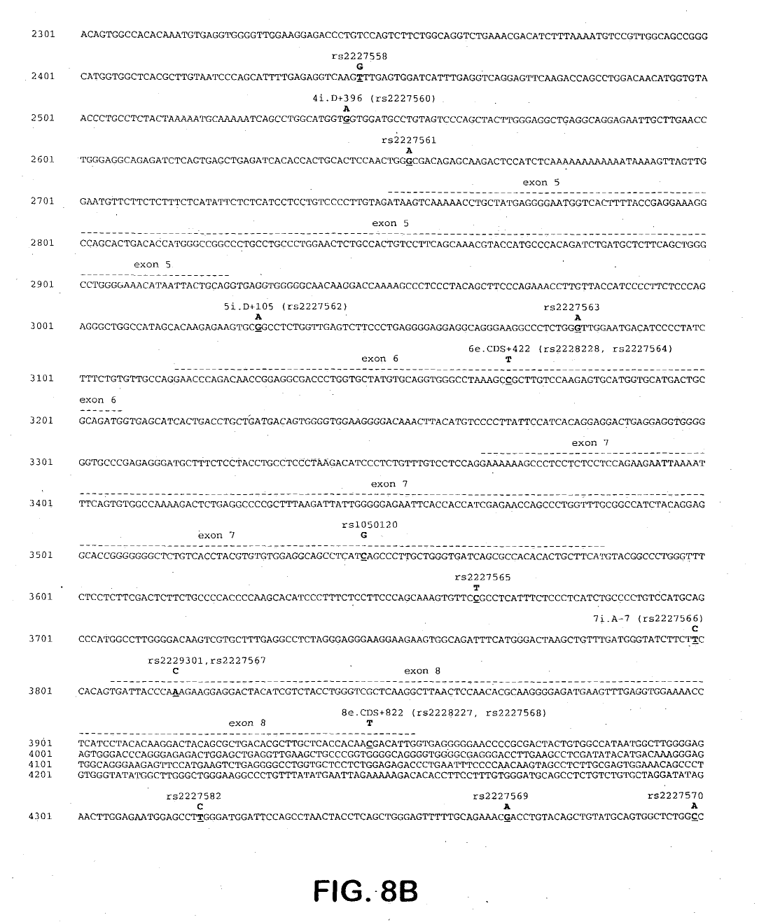

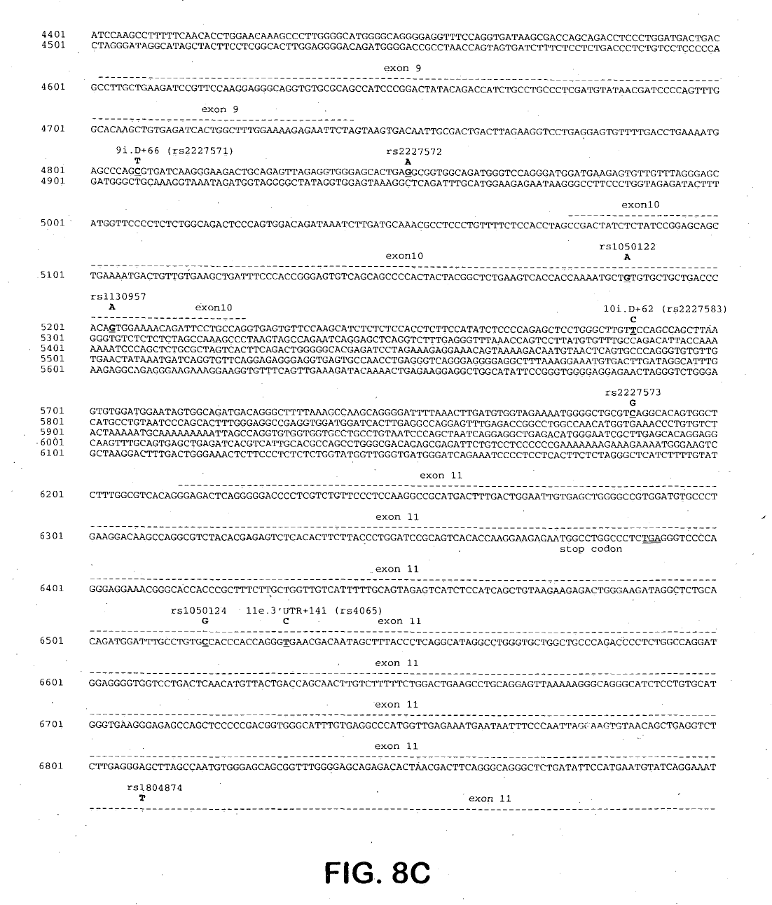

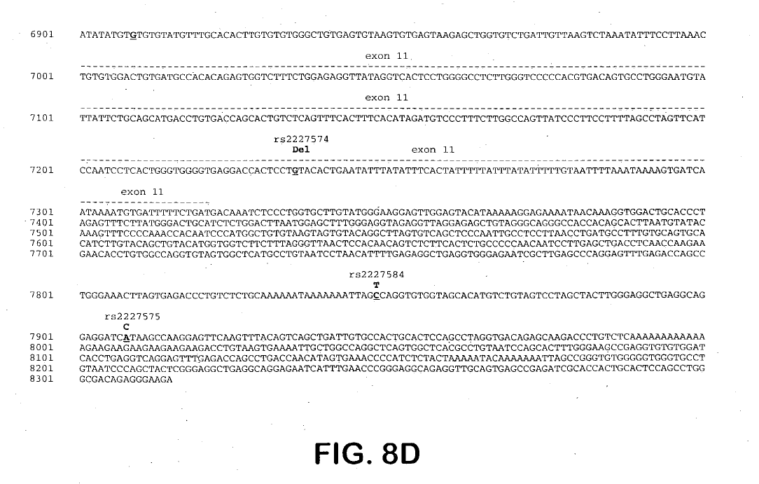

As used herein, “PLAU” refers to a urokinase plasminogen activator (uPA) gene such as the human uPA gene set forth herein in FIG. 8 and in SEQ ID NO:559.

As used herein, a “polymorphism of a gene X allele” (e.g., X=IDE, KNSL1, SNCG, TNFRSF6, LIPA or LIPA) refers to a polymorphism or polymorphic region or site of a genome that has been designated herein as being part of gene X, generally based on a determination of its position in the genome. For convenience of identification herein, polymorphisms or polymorphic regions or sites that may be located between genes (e.g., intergenic) or upstream and/or downstream of one or more genes (relative, for example to a site that is clearly identifiable as part of a particular gene) have been designated as a “gene X allele.” Such designation does not necessarily mean that the polymorphism or polymorphic region or site is a physical part of, or is a physical part only of, the one particular gene, or that it may have a functional relationship with, or role in the functioning of, the one particular gene, or the one particular gene alone. Thus, for example, a polymorphism or polymorphic region or site designated as one of an IDE gene allele herein may be functionally relevant to the IDE gene, a flanking gene, such as KNSL1, or to both genes.

As used herein, “association” with reference to the relationship between alleles and a trait or trait-causing allele refers to the deviation from the random occurrence of the allele and a trait or trait-causing allele in a haplotype in populations. An allele and a trait or trait-causing allele observed together on a chromosome more often than expected from their frequencies in the population may be referred to as associated.

In association-based methods, evidence is sought for a statistically significant association between an allele and a trait or trait-causing allele. For example, the occurrence of a disease-causing allele may be presumed by the occurrence of the disease trait. Association studies focus on population frequencies and explore the relationships among frequencies for sets of alleles and traits or trait-causing alleles. Association may be determined using a number of analytical methods, including but not limited to case-control studies, family-based association tests and haplotype analyses. Association determinations employing, typically, unaffected family members as controls and/or related disease family members (e.g., sib pairs) as affected individuals are particularly useful in accurate determination of association. Such determinations are in contrast to association studies using unrelated populations of subjects and matched controls (e.g., case-control studies), which have the advantage of being relatively simple in terms of the sample sets and statistical analyses involved but may be more susceptible to false positive (type I) and false negative (type II) errors.

As used herein, “at a nucleotide position corresponding to” refers to a position of interest (i.e., base number) in a nucleic acid molecule relative to the position in another reference nucleic acid molecule. Corresponding positions can be determined by comparing and aligning sequences to maximize the number of matching nucleotides, for example, such that identity between the sequences is greater than 95%, greater than 96%, greater than 97%, greater than 98% or greater than 99%. The position of interest is then given the number assigned in the reference nucleic acid molecule. For example, if a particular polymorphism in a gene occurs at nucleotide n of SEQ. ID. NO:X. To identify the corresponding nucleotide in another allele or isolate, the sequences are aligned and then the position that lines up with nucleotide n of SEQ. ID. NO:X is identified. Since various alleles may be of different length, the position designated may not actually be the nth nucleotide, but instead is at a position that “corresponds” to the position in the reference sequence.

As used herein, a nucleotide sequence that is complementary to the nucleotide sequence set forth in SEQ ID NO:X, refers to the nucleotide sequence of the complementary strand of a nucleic acid strand having SEQ ID NO:X, the term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO:X refers to the complementary strand of the strand having SEQ ID NO:X or to any nucleic acid having the nucleotide sequence of the complementary strand of SEQ ID NO:X. When referring to a single stranded nucleic acid having the nucleotide sequence SEQ ID NO:X, the complement of this nucleic acid is a nucleic acid having a nucleotide sequence which is complementary to that of SEQ ID NO:X.

As used herein, “APOE4” refers to the apolipoprotein E type 4 allele found to associate with and be a risk factor for AD. The APOE-4 allele consists of a single base change polymorphism (T to C) at nucleotide position 3932 (GenBank Accession No. M10065) which results in a cysteine to arginine substitution at residue 112 of the protein.

As used herein “nucleic acid” refers to polynucleotides such as deoxyribonucleic acid (DNA) and ribonucleic acid (RNA). The term should also be understood to include, as equivalents, derivatives, variants and analogs of either RNA or DNA made from nucleotide analogs, single-stranded (sense or antisense) and double-stranded polynucleotides. Deoxyribonucleotides include deoxyadenosine, deoxycytidine, deoxyguanosine and deoxythymidine. For RNA, the uracil base is uridine.

As used herein, “isolated” with reference to a nucleic acid molecule means that the nucleic acid has been separated from the genetic environment from which the nucleic acid was obtained. It may also mean altered from the natural state. For example, a polynucleotide naturally present in a living animal is not “isolated,” but the same polynucleotide separated from the coexisting materials of its natural state is “isolated”, as the term is employed herein. Thus, a polynucleotide produced and/or contained within a recombinant host cell is considered isolated. Also intended as an “isolated polynucleotide′ are polynucleotides that have been purified, partially or substantially, from a recombinant host cell-or from a native source. The terms isolated and purified are sometimes used interchangeably.

Thus, “isolated” with reference to a nucleic acid is also meant to include nucleic acid that is free of the coding sequences of those genes that, in the naturally-occurring genome of the organism (if any) immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

As used herein, the term “vector” refers to a nucleic acid molecule capable of transporting another nucleic acid to which it has been linked. One type of vector contemplated herein is an episome, i.e., a nucleic acid capable of extra-chromosomal replication. Vectors capable of autonomous replication and/or expression of nucleic acids to which they are linked are thus included within the term “vector.” Vectors capable of directing the expression of genes to which they are operatively linked are also referred to herein as “expression vectors.” Expression vectors commonly used in recombinant DNA techniques are often in the form of “plasmids” which refer generally to circular double-stranded DNA loops which, in their vector form, are not bound to the chromosome. “Plasmid” and “vector” are often used interchangeably as the plasmid is the most commonly used form of vector. Other vectors contemplated for use herein include other forms of expression vectors that serve equivalent functions and that become known in the art subsequently hereto.

As used herein, “cell” refers to a host cell that has been transformed or transfected with a vector containing one of the nucleic acid molecules described herein. Cells include both prokaryotic and eukaryotic cells including but not limited to human, plant and yeast.

As used herein, “primer” and “probe” refer to a nucleic acid molecule including DNA, RNA and analogs thereof, including protein nucleic acids (PNA), and mixtures thereof. Such molecules are typically of a length such that they are statistically unique (i.e., occur only once) in the genome of interest. Generally, for a probe or primer to be unique in the human genome, it should contain at least about 14, 16 or 18 contiguous nucleotides of a sequence complementary to or identical to a gene of interest. The probes and primers and primers provided herein can be 10, 12, 14, 16, 18, 20, 22, 24, 26, 28, 30, 35, 40, 45, 50, 60, 70, 80, 90, 100 or more nucleotides long, but not the entire length of the gene or cDNA sequence.

As used herein, “antisense nucleic acid molecule” refers to a molecule encoding a sequence complementary to at least a portion of a target nucleic acid molecule, for example RNA or DNA. The sequence is sufficiently complementary to be able to hybridize with the target nucleic acid, preferably under moderate or high stringency conditions to form a stable duplex. The ability to hybridize depends on the degree of complementarity and the length of the antisense nucleic acid. Generally, the longer the hybridizing nucleic acid, the more base mismatches with an RNA it can contain and still form a stable duplex. One skilled in the art can ascertain a tolerable degree of mismatch by use of standard procedures to determine the melting point of the hybridized complex.

As used herein, “specifically hybridizes” refers to hybridization of a probe or primer or antisense nucleic acid only to a target sequence. Those of skill in the art are familiar with parameters that affect hybridization; such as temperature, nucleic acid (probe, primer or antisense molecule) length and composition, buffer composition and salt concentration and can readily adjust these parameters to achieve specific hybridization of a nucleic acid to a target sequence. Stringency conditions include washing conditions for removing the non-specific nucleic acid and conditions that are equivalent to either high, medium, or low stringency as described below:

| | |
|---|---|
| 1) high stringency: | 0.1 × SSPE, 0.1% SDS, 65° C. |
| 2) medium stringency: | 0.2 × SSPE, 0.1% SDS, 50° C. |
| 3) low stringency: | 1.0 × SSPE, 0.1% SDS, 50° C. |

It is understood that equivalent stringencies may be achieved using alternative buffers, salts and temperatures.

As used herein, “adjacent” refers to a position 5′ or 3′ to the site of a polymorphism such that there could be nucleotides between that position and the site of the polymorphism. The term “adjacent” includes “immediately adjacent” which refers to a position 5′ or 3′ to the site of a polymorphism, such that there are no nucleotides between that position and the site of the polymorphism. “Adjacent” in the context of a primer or probe that hybridizes adjacent to a particular polymorphic region refers to a portion of a gene that would serve as an appropriate segment of the gene to which a primer or probe would be hybridized for purposes of specifically targeting the polymorphic region, as in, for example, amplifying, sequencing or conducting primer extension (such a single-base primer extension) of the specific polymorphic region alone (as compared to general random amplification or sequencing of the gene or genome). Such adjacent regions or segments include those that can be located, for example, up to and including 5, 10, 15, 20, 30, 40, 50, 60, 70, 80, 90, 100, 125, 150, 175, 200, 225, 250, 275, 300, 325, 350, 375, 400, 425, 450, 475 or 500 bp 5′ or 3′ of the polymorphic region or site, and are readily determinable and understood by those of skill in the art.

As used herein, a “complete” allele, as used in reference to a KNSL1 or IDE allele, refers to an allele that corresponds in length to the KNSL1 allele set forth as SEQ ID NO:347, or to the IDE allele set forth as SEQ ID NO:186, respectively.

As used herein, “drug used to treat Alzheimer's disease” means that the drug reduces the likelihood of the disease, delays the onset of the disease, lessens the symptoms, halts or delays progression of the disease.

As used herein, “drug used to treat a neurodegenerative disease” means that the drug reduces the likelihood of the disease, delays the onset of the disease, lessens the symptoms, halts or delays progression of the disease.

As used herein, “response” refers to the affect of a drug on a disease or disorder of a subject. A positive response indicates that the drug reduces the likelihood of the disease, delays the onset of the disease, lessens the symptoms, halts or delays progression of the disease. A negative response indicates that there is no therapeutic or a subtherapeutic reduction in the likelihood of the disease, delay in the onset of the disease, lessening of the symptoms, halting or delay of the progression of the disease.

As used herein, “combination” refers to any association between or among two or more items. The combination can be two or more separate items, such as two compositions or two collections, can be a mixture thereof, such as a single mixture of the two or more items, or any variation thereof. Thus, for example, a combination may be a collection of nucleic acids, such as probes or primers or genetic markers.

As used herein, “kit” refers to a package-that contains a combination, such as one or more primers or probes used to amplify or detect an allelic variant of one or more polymorphic regions of one or more of the uPA, SNCG, IDE, KNSL1, LIPA and/or TNFRSF6 genes, optionally including instructions and/or reagents for their use.

As used herein, “solid support” refers to a support substrate or matrix, such as silica, polymeric materials or glass. At least one surface of the support can be partially planar. Regions of the support may be physically separated, for example with trenches, grooves, wells or the like. Some examples of solid supports include slides and beads. Supports are of such composition so as to allow for the immobilization or attachment of nucleic acids and other molecules such that these molecules retain their binding ability.

As used herein, “array” refers to a collection of elements, such as nucleic acids, containing three or more members. An addressable array is one in which the members of the array are identifiable, typically by position on a solid support. Hence, in general the members of the array will be immobilized to discrete identifiable loci on the surface of a solid phase.

As used herein, “heterologous” and “foreign” are used interchangeably with respect to nucleic acid and refer to any nucleic acid, including DNA and RNA, that does not occur naturally as part of the genome in which it is present or which is found in a location or locations or amount in the genome that differ from that in which it occurs in nature or which is the result of genetic manipulation of an endogenous genome which alters the endogenous genome. Thus, heterologous or foreign nucleic acid includes any nucleic acid that is not normally found in the host genome in an identical context. It includes nucleic acid that is not endogenous to the cell and has been exogenously introduced into the cell. Examples of heterologous DNA include, but are not limited to, DNA that encodes a gene product or gene product(s) of interest, introduced for purposes of modification of the endogenous genes or for production of an encoded protein. For example, a heterologous or foreign gene may be isolated from a different species than that of the host genome, or alternatively, may be isolated from the host genome but operably linked to one or more regulatory regions which differ from those found in the unaltered, native gene. Other examples of heterologous DNA include, but are not limited to, DNA that encodes traceable marker proteins. Any nucleic acid that one of skill in the art would recognize or consider as heterologous or foreign to the cell in which it may naturally occur is herein encompassed by heterologous nucleic acid.

As used herein, “recombinant” with reference to a cell refers to a cell in which the endogenous genome has been altered from its original state by genetic manipulation of the endogenous genome. For example, a recombinant cell may be produced by introduction of exogenous nucleic acid into the cell which may integrate into the endogenous DNA or co-exist with the endogenous DNA episomally. A recombinant cell may also be one with a genome that is altered relative to the host cell used in generating the recombinant cell which may be produced, for example, by integration of exogenous nucleic acid into a host cell genome followed by subsequent elimination of all or part of the exogenous nucleic acid thereby resulting in an alteration in the genome of the host cell.

As used herein, “transgenic animal” refers to any animal, such as a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be stably integrated within a chromosome, i.e., replicate as part of the chromosome, or it may be extrachromosomally replicating DNA. In the typical transgenic animals, the transgene causes cells to express a recombinant form of a protein. However, transgenic animals in which the recombinant gene is silent are also contemplated, as for example, using the FLP or CRE recombinase dependent constructs. Moreover, “transgenic animal” also includes those recombinant animals in which gene disruption of one or more genes is caused by human intervention, including recombination and antisense techniques. “Transgenic animals” also include animals expressing one or more transgenes that encode for wild-type protein, but contain altered noncoding regions; e.g., promoter, introns, 5′-untranslated region and 3′-untranslated region.

As used herein, an “agent” or “molecule” that “modulates the expression or activity” of a protein refers to any drug, small molecule, nucleic acid (sense or antisense), siRNA, ribozyme, protein, peptide, lipid, carbohydrate, and the like, or combination thereof, that directly or indirectly changes, alters, abolishes, increases or decreases the expression and/or activity of the protein by affecting nucleic acid encoding the protein or the protein itself. For example, the activity of a uPA protein includes proteolytic activity characteristic of a serine protease, e.g., including the ability to activate plasminogen to form plasmin, and interaction of uPA with other molecules, e.g., uPAR and PAIs.

As used herein, “an agent that modulates a biological event characteristic of a disease” refers to any drug, small molecule, nucleic acid (sense and antisense), siRNA, ribozyme, protein, peptide, lipid, carbohydrate etc., or combination thereof, that directly or indirectly changes, alters, abolishes, increases or decreases a structural, molecular, or physiological event connected with the disease, e.g., AD, particularly an event that is readily assessable in an animal model. For example, with respect to AD, such events include, but are not limited to, amyloid production, amyloid deposition, neuropathological developments, learning and memory deficits and other AD-related characteristics (also see properties or characteristics of AD listed hereinabove.

As used herein, “combining” refers to contacting the candidate agent or biologically active agent with a cell or animal. The agent may be introduced into the cell or animal as a result of the combining. For example, combining a candidate agent with a cell may result in an agent traversing the plasma membrane. For an animal, combining may involve any of the standard routes of administration of an agent, e.g., oral, rectal, transmucosal, intestinal, intravenous, intraperitoneal, intraventricular, subcutaneous, intramuscular, etc., can be used.

As used herein, “transcription control region” refers to a region of a gene that controls transcription of a segment of the gene to which it is operatively linked. A transcription control region can contain specific sequences of DNA that are sufficient for RNA polymerase recognition, binding and transcription initiation. These sequences are typically referred to as promoter or core promoter sequences. Promoters, depending upon the nature of the regulation, may be constitutive or regulated. A transcription control region also refers regions that include sequences that modulate or regulate transcription, including RNA polymerase recognition and binding and transcription initiation. These sequences may be cis acting or may be responsive to trans acting factors. Included among such sequences are enhancer and silencer sequences which serve as specific sites for gene regulatory proteins. For example, such sequences can be found in the 5′ and 3′ untranslated regions (UTR) of genes. Transcription control region sequences that regulate transcription can be located thousands of bases away from an RNA polymerase binding site or transcription initiation site.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression, a DNA sequence and the regulatory sequence(s) are connected in such a way to control or permit gene expression when the appropriate molecules, e.g., transcriptional activator proteins, are bound to the regulatory sequence(s).

As used herein, “biologically active agents that modulate the expression or activity” of uPA, SNCG, IDE, KNSL, LIPA, or TNFRSF6 refers to any drug, small molecule, nucleic acid (sense or antisense), ribozyme, protein, peptide, lipid, carbohydrate, and the like, or combination thereof, that directly or indirectly changes, alters, abolishes, increases or decreases the expression of the uPA, SNCG, IDE, KNSL, LIPA, or TNFRSF6 protein by affecting nucleic acid encoding the protein or the protein itself, or directly or indirectly changes, alters, abolishes, increases or decreases an activity associated with the protein (see section on pages 123-124).

As used herein, the term “sense strand” refers to that strand of a double-stranded nucleic acid molecule associated with a gene that has the sequence of the mRNA that encodes the amino acid sequence encoded by the double-stranded nucleic acid molecule. Thus, the sense strand is the non-template strand of a double-stranded DNA molecule associated with a gene.

As used herein, the term “antisense strand” refers to that strand of a double-stranded nucleic acid molecule associated with a gene that has the complement of the sequence of the mRNA that encodes the amino acid sequence encoded by the double-stranded nucleic acid molecule. Thus, the antisense strand is the strand that contains the template for RNA synthesis.

B. Polymorphisms in Chromosome 10

The occurrence of variant forms of a particular nucleic acid sequence, e.g., a gene, is referred to as polymorphism. A region of a DNA segment in which variation occurs may be referred to as a polymorphic region or site. Provided herein are polymorphisms in chromosome 10, and, in particular, human chromosome 10. In particular embodiments, the polymorphisms are located on chromosome 10q, such as on chromosome 10q22, 10q23, 10q24, or 10q25. The polymorphic regions include, but are not limited to, regions of chromosome 10 surrounding and including genes such as SNCG, IDE, KNSL1, LIPA, TNFRSF6 and PLAU. Thus, the polymorphisms provided herein include polymorphisms in exons, introns or intervening sequences, intergenic regions and gene upstream and downstream regions, such as, for example, gene expression regulatory regions.

1. Polymorphisms

A polymorphic region can be a single nucleotide (e.g., single nucleotide polymorphism or SNP), the identity of which differs, e.g., in different alleles, or can be two or more nucleotides in length. For example, variant forms of a DNA sequence may differ by an insertion or deletion of one or more nucleotides, insertion of a sequence that was duplicated, inversion of a sequence or conversion of a single-nucleotide to a different nucleotide. Each individual can carry two different forms of the specific sequence or two identical forms of the sequence. More than two forms of a polymorphism may exist for a specific DNA marker in the population, but in one family just four forms are possible: two from each parent. Each child inherits one form of the polymorphism from each parent. Thus, the origin of each chromosome can be traced (maternal or paternal origin).

Certain polymorphisms may directly cause disease. For example, it is possible that any polymorphism, such as the uPA, SNCG, IDE, KNSL1, TNFRSF6 and LIPA polymorphisms disclosed herein, located within the primary RNA transcript may affect the processing of that transcript into a mature mRNA. For example, it has been shown that single nucleotide changes can disrupt the function of splicing enhancers located within coding sequences (Liu et al., 2001, Nat. Genetics, January:27(1):55-8). Enhancers can be disrupted by single nonsense, missense or translationally silent point mutations. Although less well understood, SNPs that function as exon splicing silencers also have been described (Fairbrother et al., 2000, Mol. Cell Biol., September;20(18):6816-25). Single nucleotide changes within exon splicing silencers could also alter splicing patterns of primary RNA transcripts. Point mutations in either enhancer or silencer sequences could lead to disease by changing the structure of the normal mRNA transcript via the deletion or inclusion of sequences not normally present. Such mutations would effectively alter the levels of the normal protein found within the cell and may even produce a protein with a new and possibly detrimental function.

Differences between polymorphic forms of a specific DNA sequence may be detected in a variety of ways. For example, if the polymorphism is such that it creates or deletes a restriction enzyme site, such differences may be traced by using restriction enzymes that recognize specific DNA sequences. Restriction enzymes cut (digest) DNA at sites in their specific recognized sequence, resulting in a collection of fragments of the DNA. When a change exists in a DNA sequence that alters a sequence recognized by a restriction enzyme to one not recognized, the fragments of DNA produced by restriction enzyme digestion of the region will be of different sizes. The various possible fragment sizes from a given region therefore depend on the precise sequence of DNA in the region. Variation in the fragments produced is termed “restriction fragment length polymorphism” (RFLP). The different sized-fragments reflecting variant DNA sequences can be visualized by separating the digested DNA according to its size on an agarose gel and visualizing the individual fragments by annealing to a labeled, e.g., radioactively or otherwise labeled, DNA “probe”. RFLPs occur on average every 10 kb.

RFLPs may be somewhat limiting in genetic analyses in that they usually give only two alleles at a locus and not all parents are heterozygous for these alleles and thus informative for linkage [see, e.g., Botstein et al. (1980) Am. J. Hum. Genet. 32:314-331]. Newer analytic methods take advantage of the presence of DNA sequences that are repeated in tandem, for a variable number of repeats, and that are -scattered throughout the human genome. The first of these described were variable number tandem repeats of core sequences (VNTRs) [Jeffreys et al. (1985) Nature 314:67-73; Nakamura et al. (1987) Science 235:1616-1622; Weber (1989) Am. J. Hum. Genet. 44:388-396]. VNTRs may be detected using unique sequences of DNA adjacent to the tandem repeat as marker probes, and digesting the DNA with restriction enzymes that do not recognize sites within the core sequence. VNTRs may also be detected using nucleic acid amplification methods. Highly informative VNTR loci have not been found on all chromosome arms, and those which have been identified are often situated near telomeres [Royle et al. (1998) Genomics 3:352-360], leaving regions of the genome out of reach of these multiallelic marker loci.

Eukaryotic DNA has tandem repeats of very short simple sequences termed SSRs (simple sequence repeat polymorphisms) such as, for example, (dC-dA)ₙ or (dG-dT)ₙ where n=10-60 (termed GT repeat). These are also referred to as short tandem repeat polymorphisms (STRPs) and microsatellite markers. The (dG-dT) repeats occur every 30-60 kb along the genome [Weber et al. (1989) Am. J. Hum. Genet. 44:388-396; Litt et al. (1989) Am. J. Hum. Genet. 44:397-401], and Alu 3′ (A)n repeats occur approximately every 5 kb [Economou (1990) Proc. Natl. Acad. Sci. U.S.A. 87:2951-41. Repeat polymorphisms include dinucleotide, trinucleotide and tetranucleotide repeats. Dinucleotide repeats are informative and fairly prevalent in the genome. The small size of the repeat brings about diversity of its allele sizes and thus there is a greater chance that any one person will be heterozygous for the marker. Trinucleotide and tetranucleotide repeats are repeats of three and four nucleotides.

Oligonucleotides corresponding to flanking regions of these repeats may be used as primers for the polymerase chain reaction (PCR) [Saiki (1988) Science 239:484-491] on a small sample of DNA. By amplifying the DNA with labeled, e.g., radioactive or fluorescent, nucleotides, the sample may be quickly resolved on a sequencing gel and visualized by known methods, e.g., autoradiography or fluorescence detection. Because these polymorphisms are comprised of alleles that may differ in length by only a few base pairs, they generally are not detectable by conventional Southern blotting as used in traditional RFLP analysis. The use of PCR to characterize SSRs such as GT polymorphic markers enables the use of less DNA, typically only ten nanograms of genomic DNA is needed, and is faster than standard RFLP analysis, because it essentially only involves amplification and electrophoresis.

Microsatellites have been used extensively in linkage analysis (see, e.g., carbon .wi.mit.edu:8000/cgi-bin/contig/phys_map; www.chlc.org/; gdb.infobiogen.fr/gdb/contact.html#baltimore). They have many alleles and therefore are highly informative. Although microsatellites may be used in fine mapping and association analysis, they may have one or more features that should be considered in connection with such use. For example, the large number of alleles may become a consideration when using haplotype-based methods, they are not usually intragenic, and they may have relatively high and variable mutation rates which may affect linkage disequilibrium between a marker and disease mutation.

SNP markers may also be used in fine mapping and association analysis, as well as linkage analysis [see, e.g., Kruglyak (1997) Nature Genetics 17:21-24]. Although an SNP may have limited information content, combinations of SNPs (which individually occur about every 100-300 bases) may yield informative haplotypes. SNP databases are available (see, e.g., www.ibc.wustl.edu/SNP/; www.ncbi.nlm.nih.gov/SNP/;www. genome.wi.mit.edu/SNP/human/index.html). Assay systems for determining SNPs include synthetic nucleotide arrays to which labeled, amplified DNA is hybridized [see, e.g., Lipshutz et al. (1999) Nature Genet. 21:2-24; single base primer extension methods [Pastinen et al. (1997) Genome Res. 7:606-6141, mass spectroscopy on tagged beads, and solution assays in which allele-specific oligonucleotides are cleaved or joined at the position of the SNP allele, resulting in activation of a fluorescent reporter system [see, e.g., Landegren et al. (1998) Genome Res. 8:769-776].

There are polymorphisms of chromosome 10 gene regions, in particular, chromosome 10q gene regions (e.g., chromosome 10q22, 10q23, 10q24 or 10q25 gene regions) that provide for allelic variants of the genes. Particular genes include the IDE, SNCG, KNSL1, LIPA, TNFRSF6 and PLAU genes. Some of the variants encode polymorphic proteins. Polymorphisms of the human IDE, SNCG, KNSL1, LIPA, TNFRSF6 and PLAU gene regions, in particular, exist. Provided herein are polymorphisms of IDE, SNCG, KNSL1, LIPA, TNFRSF6 and PLAU gene regions and isolated nucleic acid molecules containing polymorphisms of these gene regions. Included among the polymorphisms provided herein are polymorphisms of an IDE gene and surrounding region of chromosome 10 described in EXAMPLE 3 and included in EXAMPLE 3, Tables 4 and 4-B, polymorphisms of an SNCG gene and surrounding region of chromosome 10 described in EXAMPLE 2 and included in EXAMPLE 2, Table 2, polymorphisms of a KNSL1 gene and surrounding region of chromosome 10 described in EXAMPLE 3 and included in EXAMPLE 3, Tables 6 and 6-B, polymorphisms of a LIPA gene and surrounding region of chromosome 10 described in EXAMPLE 3 and included in EXAMPLE 3, Table 10, polymorphisms of aTNFRSF6 gene and surrounding region of chromosome 10 described in EXAMPLE 3 and included in EXAMPLE 3, Table 8, and polymorphisms of a PLAU gene and surrounding region of chromosome 10 described in EXAMPLE 4 and included in EXAMPLE 4, Tables 12 and 12-B.

Also provided and described herein below are polymorphisms of an IDE, KNSL1, SNCG, LIPA, TNFRSF6 or PLAU gene, or surrounding regions of chromosome 10, that are associated, individually and/or in combination, with a disease or disorder. In a particular example, the disease or disorder may be a neurodegenerative disease or disorder, such as, for example, Alzheimer's disease.

2. Polymorphisms as Genetic Markers

Polymorphisms may be genetic markers. A genetic marker is a DNA segment with an identifiable location in a chromosome. Genetic markers may be used in a variety of genetic studies such as, for example, locating the chromosomal position or locus of a DNA sequence of interest, identifying genetic associations of a disease, and determining if a subject is predisposed to or has a particular disease.

Because DNA sequences that are relatively close together on a chromosome tend to be inherited together, tracking of a genetic marker through generations in a family and comparing its inheritance to the inheritance of another DNA sequence of interest can provide information useful in determining the relative position of the DNA sequence of interest on a chromosome. Genetic markers particularly useful in such genetic studies are polymorphic. Such markers also may have an adequate level of heterozygosity to allow a reasonable probability that a randomly selected person will be heterozygous.

As described in Example 1, microsatellite markers, including D10S583, as well as other markers on chromosome 10q, particularly markers on chromosome 10q22-q26, are linked to AD. The peak linkage occurs on the distal approximately 70-85 cM of the q arm of chromosome 10, from about 85 cM extending distally to qter. In terms of the cytogenetic map of chromosome 10, the peak linkage extends from 10q22 to qter. The strongest linkage is on. 10q23-q25. Linkage analysis reveals cosegregation of a marker with the disease trait within individual families, and thus provides evidence that within each family, a particular allele of a marker, such as an allele of D10S583, is relatively close to at least one DNA segment that causes AD or confers an increased susceptibility to AD. As also described in Example 1, analysis of AD-linked marker D10S583 for genetic association with AD revealed association between an ˜210 bp to 211 bp allele of D10S583 and an allele that is protective against AD. Genetic association of an AD-linked marker and a population within families having AD-affected members, be it association with affected family members or with unaffected members, reveals that there is at least one AD DNA segment or AD gene within linkage disequilibrium distance of D10S583 and that there are AD-associated marker alleles in the thus-defined region of chromosome 10 that may be used in determining a predisposition to or the occurrence of AD in an individual.

Although the markers linked to AD as described in EXAMPLE 1, including D10S583, did not reveal a significant association with risk for AD, association analyses of multiple alleles of these markers revealed a trend toward risk. A disease gene, such as an AD gene, very likely may have several variant forms that place a person at risk for the disease, as well as variant forms that decrease the risk for AD and forms that are neutral (i.e., have a relative risk of 1.0). The data from association analyses of the linked markers described in EXAMPLE 1 are consistent with the possibility of multiple risk alleles of an AD gene on chromosome 10. Thus, the association of an allele of the AD-linked marker D10S583 with unaffected AD family members is consistent with the existence of at least one DNA segment that causes AD or confers increased susceptibility to AD on chromosome 10, and, in particular, chromosome 10q22-q26, such as the region of chromosome 10q23-q25, as well as being indicative of the presence of an allele on chromosome 10 that is protective against AD.

Any other marker found to be in linkage disequilibrium with D10S583 will be associated with an allele protective against AD and thus will also be evidence of the presence of at least one DNA segment that causes AD or confers increased susceptibility to AD on chromosome 10. Therefore, based on the discovery of association between D10S583 and AD, additional markers associated with AD or a protective allele may now be identified using methods as described herein and known in the art. The availability of additional markers is of particular interest in that it will increase the density of markers for this chromosomal region and can provide a basis for identification of an AD DNA segment or gene in the region of chromosome 10q, and in particular, chromosome 10q22-q26. An AD DNA segment or gene may be found in the vicinity of the marker or set of markers showing the highest correlation with AD. Furthermore, the availability of markers associated with AD makes possible genetic analysis-based methods of determining a predisposition to or the occurrence of AD in an individual by detection of a particular allele.

The search for disease-susceptibility genes generally may be conducted using two main analytical methods: linkage analysis, in which evidence is sought for cosegregation between a locus and a putative trait locus within families, and association analysis, in which evidence is sought for a statistically significant association between an allele and a trait or a trait-causing allele [Khoury et al. (1993) Fundamentals of Genetic Epidemiology, Oxford University Press, N.Y.]. These methods can be viewed as tools which may be applied in any of several approaches to disease gene discovery. Two primary approaches to disease gene discovery are genetic localization and candidate gene studies. The candidate gene approach typically takes into account knowledge of biological processes of a disease as a basis for selecting genes that encode proteins that could be envisioned to be involved in the biological processes. For example, reasonable candidate genes for blood pressure disorders could be proteins and enzymes involved in the renin-angiotensin system. Candidate genes can be evaluated genetically as possible disease genes by linkage and/or association studies of markers in the candidate gene region. Genetic localization approaches do not require knowledge of the biological or biochemical nature of the disease. In contrast to a full candidate gene approach, which immediately restricts genetic analysis of a chromosome to a specific gene region determined by a hypothesis based on trait biology, genetic localization approaches first identify a chromosomal region in which a disease gene or DNA segment is located and then gradually reduce the size of the region in order to determine the location of the specific defective DNA segment as precisely as possible. For example, in these methods, the position of an AD DNA segment or gene may be localized by determining LOD scores for different markers on chromosome 10. Candidate gene and genetic localization approaches to disease gene discovery can be combined. For example, once a particular chromosome or chromosomal region has been identified as being linked and/or associated with a disease, candidate genes in the particular chromosome or chromosomal region can be selected and genetically evaluated as possible disease genes.

C. Selection of Candidate Genes and Discovery of Polymorphisms

Human chromosome 10 contains at least 600 genes. As described herein, six gene regions were selected as candidate disease-associated regions of chromosome 10. The genes and surrounding regions are IDE, SNCG, KNSL1, TNFRSF6, LIPA and PLAU. Criteria considered in the selection process included proximity of the genes to regions of chromosome 10 containing markers showing greatest linkage to AD (i.e., linkage peaks), such as D10S583 and D10S1671 (see Examples 1 and 2), e.g., expression in brain and gene products having one or more properties relating to one or more phenomena in neurodegenerative disease.

Discovery of polymorphisms is either by in silico discovery (database mining) or by wet discovery. Various algorithms can be applied to proprietary databases (e.g., those from Incyte Genomics (Palo Alto, Calif.); Celera Genomics (Rockville, Md.)) or public sequence databases (e.g., GenBank etc. from the National Center for Biotechnology Information (Bethesda, Md.)) for in silico discovery. Wet discovery utilizes various methods of manipulating nucleic acids to detect polymorphisms; including sequencing and comparison of sequence data, single-strand conformation polymorphism detection, immobilized mismatch-binding proteins, etc.

High throughput genomic DNA sequencing of candidate genes in DNA samples obtained from the NIMH led to the discovery of novel polymorphisms in the uPA, SNCG, IDE, KNSL1, TNFRSF6 and LIPA genes and surrounding regions in chromosome 10.

D. SNCG

The SNCG gene encodes gamma-synuclein (SNCG, a.k.a. persyn and breast cancer-specific gene 1 or BCSG1); a member of a family of small cytosolic proteins predominantly expressed in the nervous system (Lavedan (1998) Genome Res 8(9):871-880). Although their physiological functions are unknown, the synucleins may play a role in intracellular vesicular trafficking and signaling and also show chaperone-like activity. Gamma-synuclein increases the susceptibility of neurofilament-H to calcium-dependent proteases, and may participate in the regulation of neurofilament network integrity (Buchman et al. (1998) Nat Neurosci 1(2):101-103). The synucleins have been implicated in a variety of neurodegenerative disorders. A fragment of α-synuclein is the non-Aβ component of amyloid plaques in Alzheimer's disease (AD) (George et al. (1995) Neuron 15(2):361-72). Two mutations in the α-synuclein gene have been associated with familial Parkinson's disease (PD) (Polymeropoulos (1997) Science 276:2045-2047; Kruger (1998) (Letter) Nature Genet. 18:106-108; Narhi (1999) J Biol Chem 274(14):9843-6). Although one study was unable to detect β- or γ-synuclein coding sequence mutations associated with PD (Lavedan et al. (1998) DNA Research 5:401-402), all three synucleins have been found in aggregates at the sites of pathological lesions in the brains of patients with PD (Galvin et al. (1999) Proc Natl Acad Sci U.S.A. 96(23):13450-5). The synucleins are also present in brain lesions found in patients with neurodegeneration with brain iron accumulation, type 1 (NBIA 1).

Human SNCG, which maps to chromosome 1q23, is organized in 5 exons and encodes a 127-residue protein (Ninkina et al. (1998) Hum Mol Genet. 7(9):1417-1424); GenBank accession numbers AF044311 and AF037207, respectively). It is highly expressed in adult substantia nigra, thalamus, STN, hippocampus, caudate nucleus and amygdala, at a moderate level in corpus callosum, heart and skeletal muscle, and low levels in pancreas, kidney and lung. High levels of γ-synuclein have been detected in elderly adult cerebral cortex (Ninkina et al. (1998) Hum Mol Genet. 7(9):1417-1424). SNCG is also highly expressed in advanced infiltrating breast carcinoma.

Several polymorphic regions (e.g., SNPs and the like) have been identified herein in the human SNCG gene and/or surrounding region of chromosome 10, as set forth in Example 2, Table 2.

E. IDE

Alzheimer's disease (AD) is characterized by the progressive and severe accumulation in the brain of the amyloid β-protein (Aβ) (Selkoe (1999) Nature 399:A23-A31). However, little is known about how Aβ, after being secreted, is degraded and cleared from tissues. Defective degradation of Aβ would be expected to be a risk factor for the development of AD.

It has been suggested that insulin-degrading enzyme (IDE), a thiol metalloendopeptidase known to cleave insulin, glucagon, and other peptide hormones may be involved in the degradation of endogenous brain-derived Aβ peptides (Kurochkin and Goto (1994) FEBS Lett 345:33-37; McDermott and Gibson (1996) D. NeuroReport 7:2163-2166; Qiu et al. (1998) J Biol. Chem 273:32730-32738). The protease is expressed in a variety of tissues including brain and has a conformational rather than a sequence specificity for its substrates. In mammalian cells, IDE has been principally localized to the cytosol and peroxisomes. (Chesneau et al. (1997) Endocrinology 138:3444-3451). It has also been shown that intact IDE, like Aβ, can be released into the extracellular fluid by healthy cultured microglial (BV-2) cells and is also present in normal CSF (Qui et al. (1998) J Biol Chem 273:32730-32738).

In addition, it has been demonstrated that neuronal-type cells exhibit significant extracellular A,β-degrading activity that is inhibited by competitive IDE substrates and other IDE inhibitors (Vekrellis, et al. (2000) J Neurosci20(5):1657-1665). IDE has been shown to be localized in part on the cell surface of differentiated neurons as well as non-neuronal cells (Seta and Roth (1997) Biochem Biophys Res Commun 231:167-171). It was confirmed that IDE has a major role in Aβ degradation by showing that cellular over expression of wild-type but not active site-mutated IDE markedly decreases the steady-state levels of naturally secreted Aβ₄₀ and Aβ₄₂ in the medium of APP-expressing cells.

Several polymorphisms have been identified herein in the human IDE gene and/or surrounding region of chromosome 10, including the intergenic region between the IDE and KNDL1 genes. These are listed in Tables 4 and 4-B.

F. KNSL1

Numerous studies suggest that aberrant trafficking or processing of APP may play a causative role in AD. Thus, understanding the normal mechanisms of axonal transport and trafficking of APP is essential to elucidating how APP participates in the development of AD.

In neurons, APP is transported within axons by fast anterograde axonal transport from the neuronal cell bodies to the distal nerve terminals. Antisense inhibition experiments using oligonucleotides complementary to kinesin heavy chain coding sequences in hippocampal neurons suggested that axonal transport of APP requires the micro-tubule-dependent motor protein kinesin-I (Ferreira et al. (1993) J. Neurosci. 13:3112-3123; Amaratunga et al. (1995) J. Neurochem. 64:2374-2376; Yamazaki et al. (1995) J. Cell Biol. 129:431-442; Kaether et al. (2000) Mol. Biol. Cell 11:1213-1224).

Kinesin-I was the first member of the kinesin superfamily to be identified (Brady (1985) Nature 317:73-75; Vale et al. (1985) Cell 42:39-50) and is responsible for ATP-dependent movement of vesicular cargoes within cells. Kinesin-I is composed of two kinesin heavy chain (KHC) and two kinesin light chain (KLC) subunits. In mouse, there are three genes encoding KHC (KIF5A, KIF5B, and KIF5C) and three genes encoding KLC (KLC1, KLC2, and KLC3) (Rahman et al. (1998) J. Biol. Chem. 273:15395-15403; Xia et al. (1998) Genomics 52:209-213). These KHC and KLC subunits appear to associate in all possible combinations. KIF4A and KIF5C are neuron-specific isoforms, whereas KLC1 is neuronally enriched. KIF5B and KLC2 are ubiquitously expressed; the expression pattern of KLC3 is unknown.

Both KHC and KLC have distinct conserved domains. KHC has an N-terminal motor domain, a central α-helical coiled-coil stalk domain, and a globular C-terminal tail domain, perhaps involved in cargo binding or motor regulation. KLC has a conserved N-terminal coiled-coil domain that binds KHC, and a C-terminal domain that consists of 6 imperfect repeats of a 34 amino acid tetra-trico peptide repeat (TPR) module. Although the function of the TPR domain in KLC is unknown, TPR domains are involved in protein-protein interactions in a large group of structurally and functionally diverse proteins (Lamb et al. (1995) Trends Biochem. Sci. 20:257-259; Blatch and Lassie (1999) Bioassays 21:932-939) and could thus be involved in linking KLC to receptor proteins in vesicular or organellar cargoes. Since some experiments suggested that “cargo” molecules themselves might interact directly with microtubule-dependent motor proteins (Tai et al. (1999) Cell 97:877-887; Bowman et al. (2000) Cell 103(4):583-594), the association of APP with the KLC subunit of kinesin-I was investigated. The data suggest that APP transport from sites of synthesis in the neuronal cell body to sites of utilization of pathogenesis at the axonal terminus is mediated by direct binding of APP to KLC.

Human Kinesin-like protein 1 (KNSL1) is an isoform of the heavy chain of Kinesin 1. KNSL1 has a role in cytokinesis (Sawin et al. (1992) Nature 359:540-543) and may also be involved in signalling within the cell via interaction with a family of GTP binding proteins referred to as the Arf's (Blangy et al. (1995) Cell 83(7):1159-1169; Boman et al. (1999) Cell Motil Cytoskeleton 44(2): 119-1132; and Deavorus and Walker (1999) Biochem Biophys Res Commun 260(3):605-608).

Several polymorphisms have been identified herein in the human KNSL1 gene and/or surrounding region of chromosome 10, including the intergenic region between the IDE and KNSL1 gene. These are listed in Tables 6 and 6-B. G. TNFRSF6

The TNFRSF6 gene encodes tumor necrosis factor receptor superfamily, member 6, which has been mapped to 10q24.1 in humans. TNFRSF6, also referred to as the Fas antigen, APO-1, CD95, FAS, APT1 and apoptosis antigen is a protein containing 335 amino acids and a single transmembrane domain. It has a calculated molecular weight of 35,000 (Itoh et al. (1991) Cell 66:233-243). TNFRSF6 shows homology with a number of cell-surface receptors, including members of the tumor necrosis factor(TNF)/nerve growth factor receptor superfamily (Oehm et al. (1992) J. Biol. Chem. 267:10709-10715). The protein exhibits several domains important to its function, including a death domain, a ligand-binding domain and a transmembrane domain. TNFRSF6 mediates apoptosis (programmed cell death). TNFRSF6 is expressed in a limited number of tissues, including thymus, liver, ovary and heart.

Mice with the lymphoproliferation (Ipr) mutation have a defect in the Fas antigen gene, a T-to-A transversion resulting in the substitution of asparagine for isoleucine. These mice develop lymphadenopathy and a systemic lupus erythematosus-like autoimmune disease, indicating a role for the Fas antigen in the negative selection of autoreactive T cells in the thymus (Watanabe-Fukunaga et al. (1992) Nature 356:314-317). The Fas antigen is expressed on the surface of a number of normal and malignant cells, including activated human T and B lymphocytes and a variety of human lymphoid cell lines.

TNFRSF6 functions as a receptor for a cytokine ligand known as fas ligand. An adaptor molecule, fadd, recruits caspase-8 to the activated receptor. The resulting aggregate called the death-inducing signaling complex (disc) performs caspase-8 proteolytic activation. Active caspase-8 initiates the subsequent cascade of caspases (aspartate-specific cysteine proteases) mediating apoptosis. Fas-mediated apoptosis may have various roles including; the induction of peripheral tolerance, in the antigen-stimulated suicide of mature T cells, or both. Also, tumor immune escape, mediating apoptosis of inactivated T cells, negative regulation of erythropoiesis through sequential activation of ICE-like (CASP4, CASP5) and CPP32-like (CASP3) caspases, playing a role in lymphoproliferative syndrome, T cell lymphoma and Hodgkin's disease. TNFRSF6 is mutated in the death domain in non-small lung cancer and non lymphoid malignancies. Also, TNFRSF6 may be involved in Churg-Strauss syndrome and the pathogeny of autoimmune diabetes.

An increased rate of cell death occurs in neurodegenerative diseases including AD. Elevated levels of FAS have been observed in the brains of AD patients. It has been suggested that Abeta induces neuronal apoptosis in the brain and that the JNK-c-Jun-Fas ligand-Fas pathway is involved in the Abeta-induced neuronal apoptosis (Morishima et al. (2001) J. Neurosci 21(19):7551-60).

Several polymorphisms have been identified herein in the human TNFRSF6 gene and/or surrounding region of chromosome 10. These are listed in Table 8.

H. LIPA

The LIPA gene encodes lysosomal acid lipase (a.k.a. acid cholesteryl ester hydrolase and cholesterol ester hydrolase). It has been mapped to 10q23.2-q23.3. LIPA is a 399 amino acid protein with a molecular weight of 45 kd. It is crucial for the intracellular hydrolysis of cholesterol esters and triglycerides that have been internalized via receptor-mediated endocytosis of lipoprotein, a process which is central to the supply of cholesterol to cells for growth and membrane function and regulation of processes involving cholesterol flux. LIPA is important in mediating the affect of low density lipoprotein (LDL) on suppression of hydroxymethylglutaryl-CoA reductase and activation of endogenous cellular cholesterol ester formation (Brown et al. (1976) J. Biol. Chem. 251:3277-3286).

Two major human genetic disorders Wolman disease (WD) and cholesterol ester storage disease (CESD) are caused by mutations in different parts of the LIPA gene. These are autosomal recessive conditions which exhibit very low acid lipase/cholesteryl ester hydrolase activities, intralysosomal lipid accumulations and altered regulation of cholesterol production.

It has been proposed that neurodegenerative diseases, such as AD, results from disruption of cholesterol uptake and metabolism, which results in the abnormal trafficking of critical neuronal membrane proteins (Lynch C. and Mobley W. (2000) Ann NY Acad Sci 924:104-11). In addition, alterations in lipid homeostasis have been suggested to be related to both APOE and beta amyloid dysfunctions in AD (Poirier J. (2000) Ann NY Acad Sci 924:81-90).

Several polymorphisms have been identified herein in the human LIPA gene and/or surrounding region of chromosome 10. These are listed in Table 10.

I. Urokinase Plasminogen Activator (uPA)

Urokinase plasminogen activator (uPA; gene symbol=PLAU) is a serine protease which can catalyze the proteolytic cleavage of peptide bonds. For example, proteolytic cleavage of plasminogen catalyzed by uPA converts plasminogen to the active serine protease plasmin.

1. Plasminogen/Plasmin System

Plasmin is a potent trypsin-Iike protease with a wide substrate specificity. For example, plasmin is a key element in the blood fibrinolytic system in which it degrades fibrin into soluble fibrin degradation products. Plasmin is the activated form of plasminogen, which is an inactive proenzyme. Conversion of plasminogen into the active plasmin enzyme occurs via the action of two serine protease plasminogen activators (PA): tissue-type (t-PA) and urokinase-type (u-PA) plasminogen activator. The plasminogen/plasmin system plays an important role in various biological processes involving proteolysis. In addition, through interplay with integrins and the extracellular matrix protein vitronectin, the system is also involved in the regulation of cell migration and proliferation in a manner independent of proteolytic activity [Irigoyen et al. (1999) Cell. Mol. Life Sci. 56:104-132].

Tissue-type PA-mediated plasminogen activation is mainly involved in the dissolution of fibrin in the circulation [see, e.g., Collen and Lijnen (1991) Blood 78:3114-3124]. Tissue-type PA has a high affinity for fibrin, and its enzymatic activity is enhanced by fibrin binding. Urokinase-type PA is recruited to the cell membrane immediately after secretion via binding to a specific cellular receptor, the u-PA receptor (u-PAR). Urokinase-type PA results in enhanced activation of cell-bound plasminogen and plays a role in localized cell-associated proteolysis. For example, u-PA appears to be involved in the induction of pericellular proteolysis via the degradation of matrix components or via activation of latent proteases or growth factors.

Plasmin is a broad-spectrum protease which degrades many substrates in the extracellular matrix. In addition, plasmin can activate matrix metalloproteases which break down the collagen components in the matrix. Urokinase-type PA has activities that play a role in stimulation of cellular proliferation, enhancement of cellular migration, alteration of cellular adhesive properties and activation of growth factors, such as vascular endothelial growth factor (VEGF) and human growth factor (HGF). Binding of uPA to uPAR leads to signal transduction, and possible activation of gene transcription, which may mediate the involvement of uPA in cell proliferation, migration and adhesion [Reuning et al. (1998) Int. J. Oncol. 13:893-906].

A plasmin-mediated proteolytic cascade is primarily responsible for mediating the proteolysis of insoluble fibrin polymers, which constitute the major proteinaceous component of blood clots. Thus, the plasmin proteolytic cascade is also referred to as the fibrinolytic cascade. Although fibrin aggregation is crucial to the suppression of hemorrhaging from injured blood vessels, abnormal deposition of fibrin clots leads to cardiovascular diseases such as thrombosis, arterial neointima formation and atherosclerosis (see, e.g., Bini and Kudryk (1994) Thromb. Res. 75:337-341 and Blomback (1996) Thromb. Res. 83:1-75). Fibrin deposition may be a consequence of low plasminogen activation due to high expression of plasminogen activator inhibitors (PAIs) or low expression of plasminogen activators (PAs). Because plasmin is a potent trypsin-Iike protease with a wide substrate specificity, yet plays a vital role in controlling fibrin clot generation, it's formation is tightly regulated in cells primarily through the availability of plasminogen activators, such as uPA, localized activation and plasminogen activator inhibitors. Therefore, maintaining appropriate amounts of uPA protein and uPA activity levels in cells is of great physiological importance in an organism, and in particular in humans.

Because of the role of uPA in fundamental cellular processes of physiological importance which also may be associated with pathological conditions, there is a need to identify factors that may affect the activity and expression of uPA. There is also a need to identify polymorphic uPA alleles and elucidate the variant phenotypic effects of such alleles in order to identify any involvement of the variants in disease.

2. The uPA Gene and Encoded Protein

The uPA gene has been isolated from several mammalian species, including humans, [see, e.g., Nagamine et al. (1984) Nucl. Acids Res. 12:9525-9541; Riccio et al. (1985) Nucl. Acids Res. 13:2759-2771; Degen et al. (1986) J. Biol. Chem. 261:6972-6985; Degen et al. (1987) Biochemistry 26:8270-82791 and a sequence of the genomic uPA gene is known. Promoter regions of the isolated uPA genes have been studied. Based on those studies, generally, a uPA gene promoter contains a TATA box (a characteristic of regulated genes) and a GC-rich region of about 200 bases immediately upstream of the cap site (a characteristic of housekeeping genes). CAAT and GGGCGG sequences, which are recognized by transcription factors CTF and SP1, respectively, can be found upstream of the cap site. Thus, uPA gene expression may be at relatively low levels in some cells. Expression of a uPA gene may be induced by a variety of signals, such as growth factors, peptide hormones, steroid hormones, UV light and cell morphology. A uPA gene can contain an enhancer about 2 kb upstream of the cap site (from approximately −1875 to −1980) [Verde et al. (1988) Nucl. Acids Res. 16:10699-10716; Cassady et al. (1991) Nucl. Acids Res. 19:6839-6847].

Rapid turnover of uPA mRNA has been reported (Irigoyen et al. (1999) Cell. Mol. Life Sci. 56:104-132), The 3′UTR of uPA mRNA contains about 900 bases and is highly conserved between rat, mouse, cow, pig and human and appears to govern the rapid uPA mRNA turnover (Nagamine et al. (1995) In: Fibrinolysis in Disease, pp. 10-20, Glas-Grenwalt P. (ed.), CRC, Boca Raton). Three regions in the 3′UTR contribute independently to the rapid turnover of mRNA and include a sequence with a stem structure, a region that requires ongoing transcription to destabilize the transcript and an AU-rich element responsible for PKC downregulation-induced uPA mRNA stabilization (Nanbu et al. (1994) Mol. Cell. Biol. 14:4920-4928).

The human uPA gene encodes an approximately 53-kDa protein produced as a single-chain protein (scuPA or pro-uPA) [Gunzler et al. (1982) Hoppe Seylers Z. Physiol. Chem. 363:1155-11651. When secreted, pro-uPA binds to uPAR and is cleaved at the K158-1159 peptide bond by plasmin to yield an active two-chain form uPA (tcuPA or uPA). The active uPA can convert neighboring membrane-bound plasminogen to plasmin. The two peptide chains of uPA are linked by disulfide bridges, and the molecule contains three functional domains: a serine protease domain in the carboxyl terminal region, also called the B chain (approximately residues 144-411), an amino-terminal fragment, referred to as the A chain, containing a kringle domain (triple-disulfide-containing structure that binds protein; approximately residues 47-135) and an epidermal growth factor (EGF)-like domain (approximately residues 4-43) which is responsible for the specific interaction with uPAR. The activity of uPA can also be regulated by binding of PAIs and endocytosis.

Proteolytic cleavage or degradation of molecules involved in interaction of cells with their environment generates rapid and irreversible changes in the cellular microenvironment that may in turn affect the structure and function of the tissues. Thus, extracellular proteolysis can play a determining role in physiological and pathological processes [see, eg., Werb (1997) Cell 91:439-442]. Polymorphisms of the genome can lead to altered gene function, protein function and/or mRNA instability. Because uPA plays an important role in proteolysis-dependent processes in cells, polymorphisms in uPA genes may significantly affect the proper functioning of cells and systems within organisms and may be directly involved in certain diseases or disorders or may predispose an organism to a variety of diseases and disorders, especially those involving alterations in proteolytic processing of proteins, and in particular proteins that tend to form aggregates, and/or alterations in the amount of bound uPA binding partners, such as PAIs, including PAI-1, and uPAR.

3. Pathophysiology Involving the Plasminogen Activation System

There are pathophysiological conditions that involve imbalances in the plasminogen activation system, including, for example, cardiovascular diseases and tumor metastasis. For example, increased uPA protein and mRNA levels can occur in marcrophage-rich areas of necrotic atherosclerotic caps and in intimal smooth muscle cells of active atherosclerotic lesions and may contribute to macrophage and intimal smooth muscle cell migration into and within the lesion [Lupu et al. (1995) Arterioscler. Thromb. Vasc. Biol. 15:1444-1455]. Urokinase PA-mediated plasmin generation is involved in processes that underlie early initiation and progression of atherosclerosis, which include the modulation of intravascular fibrinolysis and hemostasis [Collen and Lijnen (1987) In The Molecular Basis of Blood Diseases, Stamatovannopoulos et al., eds., W. B. Saunders, Philadelphia, pp. 662-688; Lijnen and Collen (1995) Thromb. Haemost. 74:387-390], kinin activation [Habal et al. (1976) Adv. Exp. Med. Biol. 70:23-26], activation of matrix-destructive latent proteinases [Pepper et al. (1993) J. Cell Biol. 122:673-684], including metalloproteinases [Murphy et al. (1992) Matrix Suppl. 1:224-230; Jean-Claude et al. (1994) Surgery 116:472-478] and arterial wall matrix metalloproteinase [Schmitt et al. (1992) Biol. Chem. Hoppe-Seyler 373:611-622] and vascular matrix remodeling [Pedersen et al. (1995) Thromb. Haemost 73:835-8401. Urokinase PA may also play a role in angiogenesis [Bacharach et al. (1992) Proc. Natl. Acad. Sci. U.S.A. 89:19686-19690], tissue remodeling that occurs during the early stage of cardiac morphogenesis and the pericellular proteolysis involved in smooth muscle migration and atheromatous plaque formation [Jackson et al. (1992) Ann. N. Y. Acad. Sci. 667:141-150]. Urokinase PA-mediated conversion of plasminogen to plasmin in the atherosclerotic plaque can result in degradation of matrix components and thus affects plaque stability [Preissner et al. (1999) Basic Res. Cardiol. 94:315-421]. Complications of atherogenesis include myocardial infarction and stroke. In the myocardium, elevated uPA activity and increased expression of uPA mRNA have been demonstrated during ischemia (coronary artery occlusion) [Knoepfler et al. (1995) J. Mol. Cell. Cardiol. 27:1317-1324].

In addition, uPA appears to have a central role in tumor angiogenesis and metastasis [Abbanai and Mazar (2001) Surg. Onc. Clin. North Am. 10:393; Konecny et al. (2001) Clin. Cancer Res. 7:1743-1749]. Prior to metastasis, expansion of a tumor involves angiogenesis, the formation of new blood vessels. Angiogenesis is a multistep process emanating from microvascular endothelial cells. Endothelial cells resting in parent vessels are stimulated to degrade the endothelial basement membrane, migrate into the perivascular stroma, and initiate a capillary sprout. The capillary sprout expands and assumes a tubular structure. Endothelial proliferation leads to extension of the microvascular tubules, which develop into loops and then into a functioning circulatory network. The exit of endothelial cells from the parent vessel involves cell migration and degradation of the extracellular matrix (ECM) in a manner similar to cancer cell invasion of the ECM.

In the process of tumor metastasis, some tumorigenic cells acquire the capacity to leave the place of origin, penetrate blood vessels, travel to remote sites of an organism and settle in different organs. Cancer cell invasion involves interactions of cancer cells with the ECM, a dense latticework of collagen and elastin embedded in a gel-like ground substance composed of proteoglycans and glycoproteins. The ECM contains a basement membrane and its underlying interstitial stroma. Tumor invasion involves: (1) cancer cell detachment from the original location, (2) attachment to the ECM, (3) degradation of the ECM, and (4) locomotion into the ECM (see, e.g., Liotta (1986) Cancer Res. 46:1-7). After detachment, the cancer cells migrate over the ECM and adhere to ECM components such as laminin, type IV collagen and fibronectin via cell surface receptors. Cell adhesion molecules, such as integrin, have been shown to mediate cancer cell attachment to vascular endothelial cells and to matrix proteins (Mundy (1997) Cancer 80:1546-1556). The attached cancer cell then secretes hydrolytic enzymes or induces host cells to secrete enzymes which locally degrade the matrix. Matrix lysis occurs in a highly localized region close to the cancer cell surface where the amount of active enzyme is disproportionately higher than that of proteinase inhibitors in the serum, matrix, or as secreted by nearby normal cells (Liotta et al. (1991) Cell 64:327-336). Tumor aggressiveness may correlate with several classes of degradative enzymes, including heparinases, thiol-proteinases (including cathepsins B and L), metalloproteinases (including collagenases, gelatinases and stromelysins) and serine proteinases (including plasmin and urokinase plasminogen activator).

During the locomotion step of invasion, cancer cells migrate across the basement membrane and stroma through the zone of matrix proteolysis. The cancer cells then enter tumor capillaries (which arise as a consequence of specific angiogenic factors) and reach the general circulation via these capillaries. After traveling to other areas of the organism, the intravasated cancer cells adhere to and extravasate through the vascular endothelium and initiate new tumor formation. During cancer invasion, uPAR binds uPA released from surrounding cancer or stroma cells. Binding of uPA to its receptor focuses proteolytic action at the cancer cell surface. uPA converts inactive plasminogen into plasmin which degrades many ECM proteins such as fibronectin, vitronectin and fibrin thus facilitating ECM degradation, cancer cell proliferation, invasion and metastasis. uPA and uPAR are expressed in numerous tumor types including prostrate, breast, colon, glioblastoma, hepatocellular and renal cell carcinoma (de Witte et al. (1999) Br. J. Cancer 80:286-294; Hsu et al. (1995) Am. J. Pathol. 147:114-123; Mizukami et al. (1994) Clin. Immunol. Immunopathol. 71:96-104).

Inappropriate angiogenesis mediated by plasminogen activation is also involved in diseases such as, for example, diabetic retinopathy, corneal angiogenesis and Kaposi's sarcoma.

The plasminogen activation system appears to be critical in cell invasion processes and chronic inflammation. It operates both directly and in concert with the matrix-metalloproteinase system, and interactions between uPA and uPAR may be involved in eliciting chemotaxis, chemoinvasion and cell multiplication [Del Rosso et al. (1999) Clin. Exp. Rheumatol. 17:485-4981. As such, activity of uPA may affect proliferating and invading cells in inflammatory joint diseases. Thus, the plasminogen activation system may have a role in many aspects of the arthritic and rheumatic diseases, ranging from the infiltration of inflammatory cells into an affected joint, infiltration of synovial cells into underlying cartilage and remodeling of cartilage.

4. Urokinase Plasminogen Activator and Neurodegenerative Disease

Proteolytic enzymes are involved in the catabolism of peptide neurotransmitters and structural cellular proteins in normal brain. Members of the serine protease family, including uPA, may play a role in normal development and/or pathology of the nervous system. Changes in the balance between serine proteases and their inhibitors may lead to pathological states similar to those associated with neurodegenerative diseases [Turgeon and Houenou (1997) Brain Res. Brain Res. Rev. 25:85-95].

A characteristic feature of Alzheimer's disease (AD) brain is the presence of amyloid-containing plaques, a major component of which is the Aβ peptide derived from a carboxy-terminal region of amyloid precursor protein (APP). Little is known about how Aβ, after being secreted, is degraded and cleared from tissues. Defective degradation of Aβ could be a factor in the development of AD. Plasmin is capable of degrading Aβ peptides in vitro with a relative efficiency comparable to its ability to degrade fibrin peptides.

Urokinase PA is expressed in the central nervous system (CNS) in neurons and may play a major role in cell migration during development of the CNS. Expression of the uPA gene is upregulated in transgenic mice containing high levels of Aβ deposits [Tucker et al. (2000) J. Neurosci. 20:3937-39461. Increased expression of uPA may be a physiological response to these lesions. When considering the very high levels of Aβ peptides present in the AD brain, plasmin-mediated degradation of Aβ peptides could play an important role in controlling Aβ deposition and neuritic plaque formation.

Polymorphisms affecting expression of uPA in the CNS in turn affect the extent of plasminogen activation in the CNS. For example, a polymorphism in the promoter region of a uPA gene could effectively alter the level of expression of uPA mRNA in the CNS, resulting in altered levels of plasmin. Abnormal levels of uPA protein and resultant abnormal levels of activated plasmin may affect the levels of Aβ in brain and contribute significantly to the development of an AD phenotype. For example, abnormally low levels of activated plasmin may result in excess Aβ accumulation and predispose an individual to an Alzheimer's disease phenotype.

J. APOE4

Apolipoprotein E (ApoE) performs various functions as a protein constituent of plasma lipoproteins, including a role in cholesterol metabolism. In cerebral spinal fluid the Aβ binding factor was found to be APOE (Strittmatter et al. (1993) Proc. Natl. Acad. Sci. U.S.A. 90:1977-1981). The APOE-4 allele is a well-established susceptibility gene for late-onset AD. The APOE-4 allele is neither necessary or sufficient for AD, but modulates the risk of developing AD (Corder et al. (1993) Science 261:921-923; Corder et al. (1994) Nat Genet 7:180-184). The APOE-4 allele consists of a single base change polymorphism (T to C) at nucleotide position 3932 (GenBank Accession No. M10065) which results in a cysteine to arginine substitution at residue 112 of the protein.

K. Genetic Linkage

Polymorphisms in chromosome 10 located in the IDE, KNSL1, SNCG, LIPA, TNFRSF6 and PLAU genes and surrounding regions can be genetic markers that may be evaluated for linkage with disease. For example, the polymorphisms can be evaluated for linkage with an IDE-, KNSL1-, SNCG-, LIPA-, TNFRSF6- or uPA-mediated disease or a neurodegenerative disease such as Alzheimer's disease. Linkage analysis is based on establishing a correlation between the transmission of genetic markers and a specific trait throughout generations.

Genetic markers that are linked with a disease tend to cosegregate with a DNA segment associated with the disease, such as, for example, an AD gene, in families affected with the disease. The markers can be identified through any linkage assessment methods described herein or known to those of skill in the art, and provide scores or results indicative of linkage to disease when tested by such linkage determination methods. The markers may be used in a variety of methods. For example, a method for detecting the presence or absence in a subject of a polymorphism linked to a DNA segment associated with a disease such as Alzheimer's disease includes a step of analyzing the IDE, KNSL1, SNCG, LIPA, TNFRSF6 or PLAU gene or surrounding regions on chromosome 10 of the subject for a polymorphism linked to a DNA segment associated with the disease. A method for identifying a polymorphism linked to a DNA segment associated with a disease can include a step of analyzing a polymorphism in the IDE, KNSL1, SNCG, LIPA, TNFRSF6 or PLAU genes and surrounding regions on chromosome 10 for linkage to disease, such as a neurodegenerative disease, e.g., AD. A particular method would involve identifying such a linked polymorphism wherein the linkage is characterized by a significant or highly significant LOD score.

1. Basis for Genetic Linkage

The closer together two sequences are on a chromosome, the less likely that a recombination event will occur between them, and the more closely linked they are. Thus, the recombination frequency, i.e., the probability that there is a recombination event between two loci (also referred to as the recombination fraction), can be used as a measure of the genetic distance between two gene loci. A recombination frequency of 1% is equivalent to 1 map unit, or 1 centimorgan (cM), which is roughly equivalent to 1,000 kb of DNA. Loci that segregate independently within a family are unlinked and have a recombination fraction of 50%, whereas linked loci cosegregate within a family and have a recombination fraction of less than about 50%. For example, genetic markers linked to a DNA segment associated with AD on chromosome 10 may have a recombination fraction of less than about 50%, or about 45% or less, or about 40% or less, or about 35% or less, or about 30% or less, or about 25% or less, or about 20% or less, or about 15% or less, or about 10% or less, or about 5% or less or about 2.5% or less, or about 2% or less, or about 1.5% or less or about 1% or less or about 0.5% or less, or about 0.1% or less, or about 0. The particular recombination fraction depends on the particular marker.

For example, in terms of the genetic distance between a linked marker on chromosome 10 and a DNA segment associated with a disease, such as AD, on chromosome 10, the markers may be less than about 85 cM from the DNA segment, or less than about 80 cM from the DNA segment, or less than about 75 cM from the DNA segment, or less than about 70 cM from the DNA segment, or less than about 65 cM from the DNA segment, or less than about 60 cM from the DNA segment, or less than about 55 cM from the DNA segment, or less than about 50 cM from the DNA segment, or less than about 45 cM from the DNA segment, or less than about 40 cM from the DNA segment, or less than about 35 cM from the DNA segment, or less than about 30 cM from the DNA segment, or less than about 25 cM from the DNA segment, or less than about 20 cM from the DNA segment, or less than about 15 cM from the DNA segment, or less than about 10 cM from the DNA segment, or less than about 5 cM from the DNA segment, or less than about 4 cM from the DNA segment, or less than about 3 cM from the DNA segment, or less than about 2 cM from the DNA segment, or less than about 1.5 cM from the DNA segment, or less than about 1.0 cM from the DNA segment, or less than about 0.75 cM from the DNA segment, or less than about 0.5 cM from the DNA segment or less than about 0.25 cM from the DNA segment, or less than about 0.2 cM from the DNA segment or less than about 0.15 cM from the DNA segment or less than about 0.1 cM from the DNA segment. The particular distance depends on the particular marker. A linked marker in the IDE, KNSL1, SNCG, LIPA, TNFRSF6 or PLAU genes or surrounding regions on chromosome 10 may be located within a DNA segment associated with a disease, e.g., AD, and may be a polymorphism in a disease gene, such as, for example, a polymorphism in an AD gene that is responsible for a defect in an AD gene. When a marker is located within a disease gene, it is referred to as coincident with the gene.

If two loci are situated on different chromosomes, the transmission of alleles from generation to generation of each locus will be random and they are said to be “unlinked.” If two loci are situated on the same chromosome, the transmission of alleles of one locus will be affected by the presence of the other locus such that the ratios of alleles are no longer independent, and the loci are referred to as “linked.” Thus, two loci may be said to be linked when they are located relatively close together on the same chromosome. A polymorphism in an IDE, KNSL1, SNCG, LIPA, TNFRSF6 or PLAU gene or surrounding regions on chromosome 10 that is linked to a disease, such as, for example, a neurodegenerative disease such as AD, is located sufficiently close to a DNA segment associated with the disease on chromosome 10 such that the marker and DNA segment are linked.

For example, in terms of the physical distance between a linked marker and a DNA segment associated with a disease such as AD on chromosome 10, the markers may be less than about 72 Mb from the DNA segment, or less than about 65 Mb from the DNA segment, or less than about 63 Mb from the DNA segment, or less than about 59 Mb from the DNA segment, or less than about 55 Mb from the DNA segment, or less than about 50 Mb from the DNA segment, or less than about 45 Mb from the DNA segment, or less than about 40 Mb from the DNA segment, or less than about 35 Mb from the DNA segment, or less than about 30 Mb from the DNA segment, or less than about 25 Mb from the DNA segment, or less than about 20 Mb from the DNA segment, or less than about 15 Mb from the DNA segment, or less than about 10 Mb from the DNA segment, or less than about 5 Mb from the DNA segment, or less than about 2.5 Mb from the DNA segment, or less than about 1 Mb from the DNA segment, or less than about 0.5 Mb from the DNA segment, or less than about 0.1 Mb from the DNA segment, or less than about 0.05 Mb from the DNA segment, or less than about 0.01 Mb from the DNA segment, or less than about 0.005 Mb from the DNA segment, or less than about 0.001 Mb from the DNA segment. The particular distance depends on the particular marker.

Two loci are completely linked when there is no recombination between them; the same alleles or phenotypes are always transmitted together from generation to generation within a family. An intermediate state of linkage, referred to as “incomplete linkage” occurs when the transmission of alleles of two loci deviates consistently and measurably from independent assortment (e.g., random transmission of alleles located on different chromosomes) but a consistent recombination fraction nonetheless exists for the loci [see, e.g., March (1999) Mol. Biotechnol. 13:113-122].

2. Analysis of Genetic Linkage

Linkage analysis is based upon establishing a correlation between the transmission of genetic markers and that of a specific trait or trait gene throughout generations within a family. Thus the aim of linkage analysis is to detect marker loci that show cosegregation with a trait of interest in a pedigree.

a. Procedures

In conducting linkage analysis, two positions on a chromosome are followed from one generation to the next within a family to determine the frequency of recombination between them. This can be accomplished by genotyping DNA from fully informative individuals within pedigrees and counting recombinants and nonrecombinants. In a study of an inherited disease, such as AD, one chromosomal position, or locus, is marked by the disease gene and the other position is marked by a DNA sequence (referred to as genetic marker) that shows natural variation in the population, e.g., variable number of tandem repeats (VNTRs), such as minisatellites and microsatellites, single nucleotide polymorphisms (SNPs) and restriction fragment length polymorphisms (RFLPs). RFLPs are variations that modify the length of a restriction fragment. Minisatellites are tandemly repeated DNA sequences present in units of about 5-50 or more repeats which are distributed along regions of human chromosomes ranging from 0.1-20 kb in length. Microsatellites are tandemly repeated DNA sequences typically present in repeats of lesser units, e.g., up to 4 repeats, than those of minisatellites. Because microsatellites and minisatellites present many possible alleles, their informative content is very high. SNPs are densely spaced in the human genome and represent the most frequent type of variation.

Inheritance of a marker can be determined by analyzing DNA from each individual for the presence or absence of the marker whereas inheritance of the disease gene can be determined by examining whether the individual displays symptoms of the disease or is a parent of an affected individual or not. In every family, the inheritance of the genetic marker is compared to the inheritance of the disease state.

Linkage analysis may be two-point, i.e., comparing-the segregation of a marker and a disease, or multipoint, i.e., simultaneous analysis of linkage between the disease and several genetic markers. Multipoint analysis can be advantageous in mapping a disease gene. For example, the informativeness of the pedigree is usually increased in multipoint analysis. Each pedigree has a certain amount of potential information, dependent on the number of parents heterozygous for the marker loci and the number of affected individuals in the family. However, not all markers are sufficiently polymorphic as to be informative in all those individuals. If multiple markers are considered simultarneously, then the probability of an individual being heterozygous for at least one of the markers is greatly increased. In addition, an indication of the position of the disease gene among the markers may be determined in multipoint analysis. This may allow identification of flanking markers, and thus eventually allows isolation of a small region in which the disease gene resides. Examples of computer software which may be used for multipoint analysis include GENEHUNTER-PLUS [Kruglyak et al. (1996) Am. J. Hum. Genet. 58:1347; Kong and Cox (1997) Am. J. Hum. Genet. 61:11791, ASPEX [see, e.g., Badner et al. (1998) Am. J. Hum. Genet. 63:880-888; Hauser et al. (1996) Genet. Epidemiol. 13:117-137; Davis and Weeks (1997) Am. J. Hum. Genet. 61:1431-14441 and LINKAGE [see Lathrop et al. (1984) Proc. Natl. Acad. Sci. U.S.A. 81:3443-3446].

b. Linkage Measurement

Linkage may be assessed by the LOD (logarithm of an odds ratio) score method [Morton (1955) Am. J. Hum. Genet. 7:277-318; Rice et al. (2001) Adv. Genet. 42:99-113] or other acceptable statistical linkage determination [see also Ott (1991) Analysis of Human Genetic Linkage, Baltimore, London, John Hopkins; Terwilliger and Ott (1994) Handbook of Human Genetic Linkage, Baltimore, John Hopkins University Press; Strachan and Read (1996) Human Molecular Genetics,Oxford. BIOS Scientific Publishers Ltd.; Sudbery (1998) Human Molecular Genetics, Harlow, Addison Wesley Longman; Lander and Schork (1994) Science 265:2037-2048]. In linkage analysis, a series of likelihood ratios (relative odds) at various possible values of Θ, ranging from 0 (no recombination) to 0.50 (random assortment) are calculated. The computed likelihoods are usually expressed as the logarithm of the likelihood ratio (LOD). The use of logarithms allows data collected from different families to be combined by simple addition. Computer programs are available that run the analyses involved in statistical linkage determination [see, e.g., LIPED; MLINK; Lathrop et al. (1984) Proc. Natl. Acad. Sci. U.S.A. 81:3443-3446; Terwilliger and Ott (1994) Handbook of Human Genetic Linkage, Baltimore, John Hopkins University Press and linkage.rockefeller.edu/soft/list.html]. A LOD score is the logarithm of the ratio of the likelihood that two loci are linked at a given distance (or recombination fraction Θ) to the likelihood that they are not linked (recombination fraction Θ=0.5; greater than 50 cM apart). The value of Θ at which the LOD score is the highest is considered to be the best estimate of the recombination fraction, the “maximum likelihood estimate.”Positive LOD scores can be considered as evidence of linkage. Genetic markers in the IDE, KNSL1, SNCG, LIPA, TNFRSF6 or PLAU genes or surrounding regions on chromosome 10 linked to a DNA segment associated with a disease such as a neurodegenerative disease, e.g., AD, yield positive LOD scores when analyzed for linkage to the disease by a LOD score method. The positive LOD score may be greater than or equal to about 1.0, or greater than or equal to about 1.5, or greater than or equal to about 1.9, or greater than or equal to 2.0, or greater than or equal to about 2.2, or greater than or equal to about 2.6, or greater than or equal to about 2.7, or greater than or equal to about 2.8, or greater than or equal to about 3.0, or greater than or equal to about 3.12, or greater than or equal to about 3.2, or greater than or equal to about 3.5, or greater than or equal to about 4.0, or greater than or equal to about 4.5, or greater than or equal to about 5.0, or greater than or equal to about 5.4, or greater than or equal to about 5.5, or greater than or equal to about 6.0, or greater than or equal to about 6.5, or greater than or equal to about 7.0, or greater than -or equal to about 7.5, or greater than or equal to about 8.0, or greater than or equal to about 8.5, or greater than or equal to about 9.0, or greater than or equal to about 9.5, or greater than or equal to about 10.0 or greater than or equal to about 10.5, or greater than or equal to about 11.0, or greater than or equal to about 11.5, or greater than or equal to about 12.0, or greater than or equal to about 12.5, or greater than or equal to about 13.0 or greater than or equal to about 13.5, or greater than or equal to about 14.0 or greater than or equal to about 14.5, or greater than or equal to about 15.0, or greater than or equal to about 15.5, or greater than or equal to about 16.0, or greater than or equal to about 16.5, or greater than or equal to about 17.0. The particular LOD score depends on the particular marker.

Criteria have been proposed for use in categorizing linkage analysis results in terms of the extent to which the results may serve as evidence of linkage between loci. For example, by some criteria [Morton (1955) Am. J. Hum. Genet. 7:277-318], a LOD score of 1.5 or greater is considered to be “suggestive” of linkage whereas a LOD score of 3 is considered as statistically significant evidence for linkage. The significance level, α, is that which is associated with a likelihood ratio test computed to the base e X²=lod(2/n10). For a LOD score of 3, α 0.0001; for a LOD score of 1.5, α<0.004. It has also been proposed that a multipoint LOD score of 5.4 may be considered as “highly significant” evidence of linkage, whereas “significant” evidence of linkage may be viewed as a multipoint LOD score 3.6 or a two-point LOD score 3.3, and “suggestive” evidence of linkage is provided by multipoint LOD scores 2.2 or two-point LOD scores 1.9 [see, e.g., Lander and Kruglyak (1995) Nat. Genet. 11:241].

Linkage analysis methods can be used to screen the entire human genome for one or more chromosomal regions containing loci linked to a disease gene. In genome screening procedures, DNA from individual members of families in which one or more family members are trait positive is typed with respect to a set of genetic markers that includes multiple markers from each human chromosome. The resolution of the screen depends on the number of markers that are typed and the distance between the markers on each chromosome. Generally, the higher the density of the collection of markers, the higher the resolution of the mapping results. Typically, markers separated by an average distance of 10 cM or less are considered to provide for a fairly high resolution genome screen. In particular, an average marker separation of 9 cM or less is used for high-resolution mapping. The results of the typing are compared to the disease status of each individual, and these data are statistically evaluated using one or more of a variety of linkage analysis computer software programs [see, e.g., O'Connell and Weeks (1995) Nat. Genet. 11:402-408 describing the VITESSE algorithm]. Traditional LOD score analysis is a strong method for evaluating linkage in forms of a disorder showing obvious Mendelian inheritance, but weakens when the mode of transmission is complex and genetic parameters cannot be accurately specified. In such cases, statistical evaluation of genotyping data may be strengthened through use of allele sharing linkage methods in pairs of affected siblings or other relative pairs and association studies. Such methods are known in the art and/or described herein.

c. Statistical Methods in Linkage Analysis

Methods of analyzing genetic linkage are termed parametric (i.e., “model-based”) if gene frequency and penetrance must be estimated, and nonparametric otherwise. There are many models within each class.

(1) Parametric Linkage Analysis

Parametric linkage analysis [see, e.g., Ott (1991) Analysis of Human Genetic Linkage, Baltimore, London, John Hopkins and Terwilliger and Ott (1994) Handbook of Human Genetic Linkage, Baltimore, John Hopkins University Press] applied to large pedigrees with many affected individuals can be useful in the identification of highly penetrant genes. A number of computer software programs are available to conduct parametric linkage analysis [see, e.g., FASTLINK; Lathrop et al. (1984) Proc. Natl. Acad. Sci. U.S.A. 81:3443-3446; Cottingham et al. (1993) Am. J. Hum. Genet. 53:252-263; Schaffer et al. (1994) Hum. Heredity 44:225-237].

Parametric linkage analysis can be limited due to its reliance on the choice of a genetic model suitable for a particular trait, and may be difficult when applied to the analysis of complex genetic traits such as those due to the combined action of multiple genes and/or environmental factors. In the mapping of diseases lacking a clear Mendelian inheritance pattern or caused by several genes of low to moderate penetrance, it may be more suitable to utilize nonparametric analysis applied to small sets of affected relatives, such as affected sib pairs.

(2) Nonparametric Linkage Analysis

Nonparametric linkage analysis involves determining whether the inheritance pattern of a chromosomal region is not consistent with random Mendelian segregation by showing that affected relatives inherit identical copies of the region (i.e., allele sharing) more often than expected by chance. Distortions from expected ratios of allele sharing among relatives [usually sibs; see, e.g., Risch (1990) Am. J. Hum. Genet. 46:229-2411 who share a disease phenotype are tested. This form of analysis is independent of the mode of inheritance of the disease and thus is well-suited for cases in which there is not an absolute correlation between phenotype and genotype, such as in many multifactorial traits in which multiple genes may contribute to observed phenotype.

Typically, nonparametric linkage analysis is based in the analysis of the proportion of alleles shared identical by descent (IBD) between two sibs affected with a disease (affected sib pairs). The degree of agreement at a marker locus in two individuals can also be measured by the number of alleles identical by state (IBS). Nonparametric linkage analysis can be used in genome wide scans of multifactorial diseases using linkage maps of genetic markers, e.g., microsatellite markers. A number of computer software programs are available to conduct nonparametric linkage analysis [see, e.g., MAPMAKER/SIBS, Lander and Kruglyak (1995) Am. J. Hum. Genet. 57:439-454; GENEHUNTER-PLUS, Kruglyak et al. (1996) Am. J. Hum. Genet. 58:1347; SIMIBD, Davis et al. (1996) Am. J. Hum. Genet. 58:867-880; ASPEX (MLS), Risch (1990) Am. J. Hum. Genet. 46:222-253].

L. Genetic Association of Polymorphisms in Chromosome 10 Gene Regions with Disease

Genetic analyses described herein led to the discovery of genetic association of polymorphisms of human chromosome 10q with Alzheimer's disease (AD). Polymorphisms (including SNPs) of genes (such as IDE, SNCG, KNSL1, LIPA, TNFRSF6 and PLAU) and surrounding regions on chromosome 10 were analyzed individually and in combination as haplotypes for association with AD using a family-based test method for association. Both individual SNPs and haplotypes that are associated with AD or protection against AD were identified.

For example, a global test for association of a haplotype of 3 SNPs corresponding to nucleotide positions 3169, 3947 and 6532, respectively, of SEQ ID NO:559 or 560 yielded results indicative of association with AD, even after correction of the results which involved dividing the probability value by the number of tests conducted. The results of separate analysis of individual alleles of the haplotype of three SNPs for association with AD indicated a possible nominal association with protection against AD. Thus, a haplotype of polymorphisms of the uPA (i.e., PLAU) gene which is indicative of association with AD has a T, C and T nucleotide at positions 3169, 3947 and 6532, respectively of SEQ ID NO:559 or 560, or the complements thereof at the complementary positions. Similarly, association analysis of polymorphisms of the LIPA gene yielded results indicative of association of a haplotype of 3 SNPs of the LIPA gene (corresponding to nucleotide positions 1852, 6063 and 7820 of SEQ ID NO:468 wherein the nucleotides at the positions are A, G and C, respectively, or the complements in the complementary positions thereof) with protection against AD. Association analysis of polymorphisms of the IDE gene yielded results indicative of association of a haplotype of 5 polymorphisms of the IDE gene (corresponding to nucleotide positions 122260, 120416, 120288, 80752 and 54795 of SEQ ID NO:484 wherein, in a particular embodiment, the nucleotides at the positions are A, A, G, A and G, respectively, or the complements in the complementary positions thereof) with protection against AD.

These findings provide evidence of association of polymorphisms of the human chromosome 10q genes with AD. The results may also be indicative of the possible presence of an allele within the uPA, LIPA and IDE genes or within linkage disequilibrium distance of these genes on chromosome 10 that confers in those who carry the allele protection against AD relative to those who do not carry the allele.

Furthermore, the finding of global association of the haplotype of three SNPs with AD indicates that there is an allele of the uPA gene that is associated with one or more AD DNA segments or AD genes on chromosome 10, and in particular chromosome 10q, that either directly cause or confer an increased susceptibility to AD (e.g., a “risk” or “disease” allele). A protective allele generally has a counterpart disease risk allele. For example, the APOE gene, located in a peak linkage region on chromosome 19 identified in a genetic linkage analysis of late-onset AD families [Pericak-Vance et al. (1991) Am. J. Hum. Genet. 48:1034-1050], has three common alleles designated ε2, ε3 and ε4. The ε3 allele is the most common allele, and the ε2 and ε4 alleles are considered variants which affect genetic susceptibility to AD. The ε4 allele is associated with an increased risk and earlier age-at-onset whereas the ε2 allele confers a decreased risk and older age-at-onset [Corder et al. (1994) Nat. Genet. 7:180-1841. Thus, there may be multiple alleles of the uPA, LIPA and IDE genes associated with AD wherein one or more is associated with risk or increased susceptibility for AD and one or more is associated with protection against AD.

As also described herein, association analysis of polymorphisms of the IDE and KNSL1 genes led to the discovery of association of 4 separate, individual polymorphisms with risk for AD (corresponding to nucleotide positions 122260, 133354/133355 and 132370 of SEQ ID NO:484 and 41014/41015 of SEQ ID NO:347 wherein, in a particular embodiment, the nucleotides at the positions are G, a 7-bp polyT insertion between positions 133354/133355, A and an insertion of AATTT between positions 41014/41015, respectively, or the complements in the complementary positions thereof). The polymorphisms corresponding to nucleotide positions 133354/133355 and 132370 of SEQ ID NO.:484 and 41014/41015 of SEQ ID NO:347 were found to be in linkage disequilibrium. Haplotype analysis of polymorphisms of the IDE and KNSL1 genes also led to the discovery described herein of association of IDE gene haplotypes (a 5-SNP haplotype, a 4-SNP haplotype and two 3-SNP haplotypes) with risk for AD and association of a haplotype of 4 polymorphisms of the KNSL1 gene with risk for AD. Five SNPs in a haplotype of the IDE gene that associates with risk for AD correspond to nucleotide positions 122260, 120416, 120288, 80752 and 54795 of SEQ ID NO:484 wherein, in a particular embodiment, the nucleotides at the positions are G, A, G, A and G, respectively, or the complements thereof in the complementary positions. Four SNPs in a haplotype of the IDE gene that associates with risk for AD correspond to nucleotide positions 121239, 120416, 120288 and 80752 of SEQ ID NO:484 wherein, in a particular embodiment, the nucleotides at the positions are A, A, G and A, respectively, or the complements thereof in the complementary positions. In one 3-SNP haplotype of the IDE gene that associates with risk for AD, the SNPs correspond to nucleotide positions 121239, 120416 and 80752 of SEQ ID NO:484 and, in a particular embodiment, the nucleotides at the positions are C, A and A (or the complements there of in the complementary positions). In another 3-SNP haplotype of the IDE gene that associates with risk for AD, the SNPs correspond to nucleotide positions 120416, 120288 and 80752 of SEQ ID NO:484 and, in a particular embodiment, the nucleotides at the positions are A, A and A. (or the complements thereof in the complementary positions). Four polymorphisms in a haplotype of the KNSL1 gene that associates with risk for AD correspond to nucleotide positions 132370, 133354/133355, 147842 and 178980/178981 of SEQ ID NO:484 wherein, in a particular embodiment, the nucleotides at the positions are A, a 7-bp polyT insertion between positions 133354/133355, the presence of the sequence AGTT at positions 147842-147845, and an insertion of the sequence AATTT between positions 178980/178981, respectively, or the complements thereof in the complementary positions. Similar to the polymorphisms in Table 14 herein that are in linkage disequilibrium with SNP 122260 of SEQ ID NO:484, the polymorphisms that are in linkage disequilibrium with position 178980/178981 of SEQ ID NO:484 (a position associated with AD) are also associated with AD, and may therefore be assessed in place of assessing the polymorphism at position 178980/178981 of SEQ ID NO:484 for each of the methods and applications provided herein that assess the polymorphism at position 178980/178981 of SEQ ID NO:484.

Based on the discovery of association between polymorphisms of genes and surrounding regions of chromosome 10q, as described herein, additional polymorphisms associated with AD located, for example, in the IDE, KNSL1, LIPA, PLAU, SNCG and/or TNFRSF6 genes and surrounding regions, may now be identified using methods as described herein and known in the art. The availability of additional AD-associated polymorphisms is of particular interest in that it will increase the density of markers for this chromosomal region, facilitate the identification of polymorphisms or mutations involved in pathogenesis of disease (in particular neurodegenerative disease such as AD) and can provide a basis for possible genetic analysis-based methods of determining a level of risk for AD and/or a predisposition to or the occurrence of AD in an individual by detection of a particular allele.

The discovery of association between polymorphisms of chromosome 10q, including polymorphisms of the IDE, KNSL1, LIPA and PLAU genes or surrounding regions, and AD thus provides a basis for genetic analysis methods described herein which include: methods for genotyping an individual, methods for identifying polymorphisms associated with a disease, such as a neurodegenerative disease including AD; methods for detecting polymorphisms associated with a disease, such as a neurodegenerative disease including AD; methods for detecting the presence of a DNA segment associated with a disease, such as a neurodegenerative disease including AD, in a subject; methods for determining the level of risk for a disease, such as a neurodegenerative disease including AD, in a subject; methods for determining a predisposition to and/or the occurrence of a disease, such as a neurodegenerative disease including AD, in a subject; methods for identifying a region or regions of the human genome containing a disease DNA segment or gene, such as a neurodegenerative disease DNA segment or gene, including an AD DNA segment or AD gene; methods for predicting response to treatment for a disease, such as a neurodegenerative disease including AD; methods for treating a disease, such as a neurodegenerative disease including AD; and methods for identifying a disease gene, such as a neurodegenerative disease, e.g., AD, gene. Also provided herein are compositions that may be used in methods described herein, such as nucleic acids that may be used as probes or primers for detection of polymorphisms associated with a disease, such as a neurodegenerative disease including AD, and combinations, kits and articles of manufacture containing the nucleic acids. Such compositions may also be used in methods of determining a predisposition to and/or the occurrence of a disease, such as a neurodegenerative disease including AD, in a subject.

Polymorphisms of the IDE, KNSL1, SNCG, LIPA, TNFRSF6 and PLAU genes and surrounding regions may be analyzed individually and in combinations, e.g., haplotypes, for genetic association with diseases or disorders or protection against diseases or disorders. Such diseases and disorders include an IDE-, KNSL1-, SNCG-, TNFRSF6-, LIPA- and/or uPA-mediated disease or disorder. For example, polymorphisms of these genes, individually and/or in combination, may be associated with a disease or disorder involving proteolysis, protein or peptide degradation, and/or interactions between the proteins encoded by these genes and other molecules. Particular diseases and disorders with which the polymorphisms may be associated include neurodegenerative disorders such as Alzheimer's disease and late-onset AD. Other diseases and disorders include insulin degrading enzyme-mediated diseases and disorders such as diseases involving insulin-regulation and peptide signalling, kinesin-Iike protein 1-mediated diseases and disorders such as mitotic disorders and diseases involving cell signalling and membrane transport, tumor necrosis factor receptor superfamily member 6-mediated diseases such as diseases involving apoptosis, lymphoproliferative diseases,. T cell lymphoma, Hodgkin's disease, non-small cell lung cancer, non-lymphoid malignancies, Churg-Strauss syndrome and autoimmune diseases, lysosomal acid lipase-mediated diseases such as lipid and cholesterol regulation diseases, gamma synuclein-mediated diseases such as diseases involving intracellular vesicular trafficking and cell signalling and urokinase plasminogen activator-mediated diseases. Urokinase plasminogen activator-mediated diseases include, for example, a disease or disorder involving proteolysis and/or involving interactions between uPA and other molecules, e.g., uPAR and PAIs. Particular diseases include, but are not limited to, thrombosis, thrombolytic diseases, stroke, atherosclerosis, coronary artery disease, cardiovascular disease, cardiac disorders, myocardial infarction, cardiomyopathies, proliferative diseases, cancer, tumor angiogenesis, tumor metastasis, arthritis, rheumatic diseases or inflammatory diseases, such as inflammatory joint diseases.

Thus, provided herein are methods of identifying polymorphisms associated with diseases and disorders. The methods involve a step of testing polymorphisms of an IDE, SNCG, KNSL1, LIPA, TNFRSF6 or PLAU gene, or surrounding region, and in particular a human gene, individually or in combination, e.g., haplotypes, for association with a disease or disorder. In particular embodiments, the polymorphisms analyzed, individually and/or in combinations, are those listed in the EXAMPLES and Tables herein.

The analysis or testing may involve genotyping DNA from individuals affected with the disease or disorder, and possibly also from related or unrelated individuals, with respect to the polymorphic marker and analyzing the genotyping data for association with the disease or disorder using methods described herein and/or known to those of skill in the art. For example, statistical analysis of the data may involve a chi-squared or Fisher's exact test and may be conducted in conjunction with a number of programs, such as the transmission disequilibrium test (TDT), affected family based control test (AFBAC),and the haplotype relative risk test (HRR). Case-control strategies can be applied to the testing, as can, for example, TDT approaches.

Also provided herein are polymorphisms of chromosome 10q, particularly human chromosome 10q and in the region containing the IDE, KNSL1, LIPA and PLAU genes, associated with AD. In particular embodiments, the AD is late-onset AD. The polymorphisms can be over-represented in cases in case-control studies and/or can be associated with affected individuals in a family-based association analysis. Alternatively, the polymorphisms can be under-represented in cases in case-control studies and/or associated with unaffected individuals in a family-based association analysis. The polymorphisms can be identified through linkage disequilibrium or association assessment methods described herein or known to those of skill in the art, and provide scores or results indicative of linkage disequilibrium with an AD DNA segment or gene or of association with AD when tested by such assessment methods. The polymorphisms are associated with AD as individual markers and/or in combinations, such as haplotypes, that are associated with AD.

Also provided herein are combinations of polymorphisms which are associated with AD. In one embodiment, each polymorphism in a combination is associated with AD. In other embodiments, some of the polymorphisms in the combination are associated with AD and some of the polymorphisms are not or none of the polymorphisms is associated with AD. In such embodiments, the combination of polymorphisms as a whole is associated with AD, such as in the case of a haplotype and in particular a globally associated haplotype.

1. Genetic Association

When two loci are extremely close together, recombination between them is very rare, and the rate at which the two neighboring loci recombine can be so slow as to be unobservable except over many generations. The resulting allelic association is generally referred to as linkage disequilibrium. Linkage disequilibrium can be defined as specific alleles at two or more loci that are observed together on a chromosome more often than expected from their frequencies in the population. As a consequence of linkage disequilibrium, the frequency of all other alleles present in a haplotype carrying a trait-causing allele will also be increased (just as the trait-causing allele is increased in an affected, or trait-positive, population) compared to the frequency in a trait-negative or random control population. Therefore, association between the trait and any allele in linkage disequilibrium with the trait-causing allele will suffice to suggest the presence of a trait-related DNA segment in that particular region of a chromosome. On this basis, association studies are used in methods of locating and discovering disease-susceptibility genes.

A marker locus must be tightly linked to the disease locus in order for linkage disequilibrium to exist between the loci. In particular, loci must be very close in order to have appreciable linkage disequilibrium that may be useful for association studies. Association studies rely on the retention of adjacent DNA variants over many generations in historic ancestries, and, thus, disease-associated regions are theoretically small in outbred random mating populations. In practice, however, it is common to find some degree of linkage disequilibrium between alleles that are up to about 0.1-0.3 cM apart, or about 1 to 2 cM apart, or even 3 to 4 cM apart, and this can be used for disease gene mapping [Jorde (1995) Am. J. Hum. Genet. 56:11-14; Xiong and Guo (1997) Am. J. Hum. Genet. 60:1513-1531; Reich et al. (1995) Am. J. Hum. Genet. 56:11-14]. In contrast, linkage studies, by relying on identification of haplotypes that are inherited intact over several generations (such as in families or pedigrees of known ancestry) focus on recent, usually observable ancestry in which there have been relatively few opportunities for recombination to occur. Thus, disease gene regions identified by linkage will often be large, encompassing many tens of megabases of DNA.

The power of genetic association analysis to detect genetic contributions to complex disease can be much greater than that of linkage studies. Linkage analysis can be limited by a lack of power to exclude regions or to detect loci with modest effects. Association tests can be capable of detecting loci with smaller effects [Risch and Merikangas (1996) Science 273:1516-15171 which may not be detectable by linkage analysis. Studies based on pedigrees may only narrow the location of a trait-causing allele; in such cases, association may be a powerful method for fine-scale mapping which can serve to further refine the location of the allele.

The aim of association studies when used to discover disease-susceptibility genes is to identify particular genetic variants that correlate with the disease phenotype at the population level. The aim of association studies when used to discover genes that are protective against a disease, such as AD, is to identify particular genetic variants that correlate with unaffected individuals at the population level. Association at the population level may be used in the process of identifying a disease-susceptibility gene or DNA segment because it provides an indication that a particular marker is either a functional variant underlying the disease (i.e., a polymorphism that is directly involved in causing a particular trait) or is extremely close to the disease gene on a chromosome. When a marker analyzed for association with a disease is a functional variant, association is the result of the direct effect of the genotype on the phenotypic outcome. When a marker being analyzed for association is an anonymous marker, the occurrence of association is the result of linkage disequilibrium between the marker and a functional variant. Association analysis can also be used to identify an allele that is either a functional variant that is protective against a disease, such as AD, or an allele that is extremely close to a gene that confers protection against the disease.

There are a number of methods typically used in assessing genetic association as an indication of linkage disequilibrium, including the epidemiological case-control study of unrelated subjects and methods using family-based controls. Although the case-control design is relatively simple, it is the most prone to identifying DNA variants that prove to be spuriously associated (i.e., association without linkage) with disease [Cardon and Bell (2001) Nature Rev. Genet. 2:91-99]. Spurious association can be due to the structure of the population studied rather than to linkage disequilibrium. Thus, for example, if cases and controls are not ethnically comparable, then differences in allele frequency can emerge at loci that differentiate the groups whether the alleles are causally related to disease or not (a phenomenon referred to as population stratification). Linkage analysis of such spuriously associated allelic variants, however, would not detect evidence of significant linkage because there would be no familial segregation of the variants. Therefore, putative association between a marker allele and a disease trait identified in a case-control study should be tested for evidence of linkage between the marker and the disease before a conclusion of probable linkage disequilibrium is made. Association tests that avoid some of the problems of the standard case-control study utilize family-based controls in which parental alleles or haplotypes not transmitted to affected offspring are used as controls.

In contrast to genetic linkage, which is a property of loci, genetic association is a property of alleles. Association analysis involves a determination of a correlation between a single, specific allele (or all combinations of particular alleles of a haplotype when performing a global association analysis) and a trait across a population, not only within individual families. Thus, a particular allele found through an association study to be in linkage disequilibrium with a disease allele can form the basis of a method of determining a predisposition to or the occurrence of the disease in any individual.

Generally, detecting an association between a genotype and a phenotype involves the steps of: a) determining the frequency of at least one polymorphism in a trait positive population by genotyping; b) determining the frequency of the related polymorphism in a control population; and c) determining whether a statistically significant association exists between the genotype and the phenotype.

The control population may be a trait negative population, or a random population. Each of the genotyping steps a) and b) may be performed on a pooled biological sample derived from each of the populations or each of the genotyping of steps a) and b) is performed separately on biological samples derived from each individual in the population or a subsample thereof.

The general strategy to perform association studies using polymorphisms derived from a region carrying a candidate gene is to scan two groups of individuals (case-control populations) in order to measure and statistically compare the allele frequencies of the markers in both groups.

If a statistically significant association with a trait is identified for at least one or more of the analyzed polymorphisms, one can assume that either the associated allele is directly responsible for causing the trait (i.e., the associated allele is the trait-causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait-causing allele but is in linkage disequilibrium with the real trait-causing allele, then the trait-causing allele can be found by sequencing the vicinity of the associated marker, and performing further association studies with the polymorphisms that are revealed in an iterative manner.

Association studies are usually run in two successive steps. In a first phase, the frequencies of a reduced number of markers from the candidate gene are determined in the trait positive and control populations. In a second phase of the analysis, the position of the genetic loci responsible for the given trait is further refined using a higher density of markers from the relevant region. However, if the candidate gene under study is relatively small in length, a single phase may be sufficient to establish significant associations.

2. Methods Used in Association Analyses

Association studies explore the relationships among frequencies for sets of alleles between loci. Association studies may be conducted within the general population and are not limited to studies performed on related individuals in affected families. There are several methods for testing for genetic association.

a. Case-control Studies

The simplest form of association analysis is the case-control study in which unrelated populations of affected (or trait-positive) subjects (i.e., case individuals) and unrelated control (unaffected, trait-negative or random) individuals are analyzed. Such population-based association studies do not concern familial inheritance but compare the prevalence of a particular genetic marker or set of markers in case-control populations. Marker allele frequencies in each population may be compared using a chi-squared or Fisher's exact test (see, e.g., linkage.rockefeller.edu/software/utilities).

The control group is typically “matched” as much as possible to the case population, particularly to avoid problems of population stratification. Thus, the control group may be ethnically matched to the case population and matched for the main known confusion factor for the trait under study (e.g., age-matched for an age-dependent trait). An important step in the dissection of complex traits using association studies is the choice of case-control populations [see Lander and Schork (1994) Science 265:2037-2048]. A major step in the choice of case-control populations is the clinical definition of a given trait or phenotype. A genetic trait may be analyzed by association methods by carefully selecting the individuals to be included in the trait-positive and trait-negative phenotypic groups. Several criteria are often useful: clinical phenotype, age at onset, family history and severity. The selection procedure for continuous or quantitative traits (such as blood pressure, for example) involves selecting individuals at opposite ends of the phenotype distribution of the trait under study, so as to include in these trait-positive and trait-negative populations individuals with non-overlapping phenotypes. Preferably, case-control populations contain phenotypically homogeneous populations. Trait-positive and trait-negative populations contain phenotypically uniform populations of individuals representing each between 1 and 98%, or between 1 and 80%, or between 1 and 50%, or between 1 and 30%, or between 1 and 20% of the total population under study, and preferably selected among individuals exhibiting non-overlapping phenotypes. The clearer the difference between the two trait phenotypes, the greater the probability of detecting an association with markers. The selection of those drastically different but relatively uniform phenotypes enables efficient comparisons in association studies and the possible detection of marked differences at the genetic level, provided that the sample sizes of the populations under study are significant enough.

Allelic frequencies of markers in populations can be determined by genotyping pooled DNA samples or individual samples. When each individual is genotyped separately, simple gene counting may be applied to determine the frequency of an allele or of a genotype in a given population. The proportional representation of the allele for the population is then determined.

The allelic frequencies of the marker in case and control populations are analyzed to determine whether a statistically significant association exists between the genotype and phenotype. The statistical significance of a correlation between phenotype and genotype may be determined by any statistical test known in the art and with any accepted threshold of statistical significance being required. The application of particular methods and thresholds of significance are well within the level of skill of one skilled in the art. A commonly used statistical test is a chi-square test with one degree of freedom. A P-value is calculated, which is the probability that a statistic as large or larger than the observed one would occur by chance. If a statistically significant association with a trait is identified for at least one or more of the analyzed markers, it may be assumed that either the associated allele is directly responsible for causing the trait (i.e., the associated allele is the trait-causing allele), or more likely the associated allele is in linkage disequilibrium with the trait-causing allele.

In testing the association of a particular allele against the disease phenotype, it may be useful to correct the results. One such correction method is referred to as the “Bonferroni” correction in which the probability value required to give significance is divided by the number of tests conducted. For example, if five markers are tested, each with five alleles, a probability value of 0.002 would be required to declare significance at the 5% level. A P value can generally be considered statistically significant if it is less than or equal to about 1×10⁻², or less than or equal to 0.05 or less than 0.05. In carrying out association studies with the polymorphisms (polymorphic regions) described herein for the SNCG, KNSL1, IDE, LIPA, TNFRSF6 and PLAU genes and surrounding regions, significant associations between the polymorphic markers or allelic variants and disease, such as a neurodegenerative disease, e.g., AD, can be revealed and used as the basis for many methods employing the variants, for example, diagnostic, pharmacogenomic and drug-screening methods.

Case-control studies can be susceptible to false positive (type I) and false negative (type II) errors. Thus, a negative result may mean a lack of association or a false negative due to insufficient power to detect association. A positive result may mean an allelic association with disease, the presence of an unknown factor such as population stratification between cases and controls or a false positive due to an insufficient sample size for the tests being conducted. Calculators (see, e.g., www.stat.ucla.edu/calculators/powercalc/binomial/case-control/b-case-control-samp.html) are available to estimate required sample size for a given marker frequency, relative risk of interest, power and significance level (corrected if necessary for multiple tests).

Typical association studies based on candidate genes, and in particular, case-control studies, may have a limited ability to discern true medium-sized signals from false positives [see, e.g., Emahazion et al. (2001) Trends Genet. 17:407-413]. Thus, reports of positive association findings frequently cannot be replicated.

b. Case-control Studies Using Family-based Controls

Case-control studies using family-based controls have been developed to address possible errors relating to inadequate matching of unrelated cases and controls. Unlike case-control tests, family-based tests are not affected by population stratification, which can lead to spurious associations of a marker allele with disease susceptibility. Such analytical techniques include the transmission disequilibrium test (TDT) [Spielman et al. (1993) Am. J. Hum. Genet. 52:506-516], affected family based control test (AFBAC) [Thomson (1995) Am. J. Hum. Genet. 57:487-498 and Schaid and Sommer (1994) Am. J. Hum. Genet. 55:402-409] and the haplotype relative risk test (HRR) [Falk and Rubinstein (1987) Ann. Hum. Genet. 51:227-233; Terwilliger and Ott (1992) Hum. Hered. 42:337-3461. In these methods, family members (usually unaffected) can be used as internal controls. In the HRR and AFBAC tests, an affected individual and two parents are typed for a marker hypothesized to have an allele associated with the disease. The number of control alleles are derived from the parental alleles not transmitted to the affected child, and these are compared to the number of alleles transmitted to the affected child by a chi-squared test. In the TDT test, one of the parents must be heterozygous for the marker concerned, and the comparison is made between the alleles that are transmitted to the affected child and those that are not. Deviation from the expected Mendelian 50% transmission is tested by a chi-squared or Fisher's exact test.

The TDT focuses on alleles transmitted to affected offspring, but is formulated to take account of both the linkage and the disequilibrium that underlie the association. Depending on the data structure, TDTs are tests of either linkage or linkage and association. The proposed test statistic is a McNemar's chi-square and tests the null hypothesis that the putative disease-associated alleles transmitted 50% of the time from heterozygous parents; under the alternative hypothesis, the disease-associated allele will be transmitted more often.

The TDT has been extended to take into account multiallelic marker loci [Spielman and Ewens (1996) Am. J. Hum. Genet. 59:983-989; Sham and Curtis (1995) Ann. Hum. Genet. 59:323-336; Bickeboeller and Clerget-Darpoux (1995) Genet. Epidemiol. 12:865-870; and Rice et al. (1995) Genet. Epidemiol. 12:659-6641, the availability of only one parent [Sun et al. (1999) Am. J. Epidemiol. 150:97-104], analysis of affected sibs or trios [Martin et al. (1997) Am. J. Hum. Genet. 61:439-448], multiple analysis of linked alleles in haplotypes [Clayton and Jones (1999) Am. J. Hum. Genet. 65:1161-1169 and Clayton (1999) Am. J. Hum. Genet. 65:1170-11771, pooled genotyping of affected children [Risch and Teng (1998) Genome Res. 8:1273-12881 and transmission from parents homozygous at a tightly linked locus [Lie et al. (1999) Am. J. Hum. Genet. 64:793-800]. Family-based tests, such as TDT, have largely required knowledge of parental marker genotypes; however, for late-onset diseases, parental data are often not available. There are tests of linkage and association that use unaffected siblings as surrogates for untyped parents from which probable parental genotypes may be derived [Spielman and Ewens (1998) Am. J. Hum. Genet. 62:450-458 (also referred to as the sib-TDT or S-TDT); Horvath and Laird (1998) Am. J. Hum. Genet. 63:1886-1897; Boehnke and Langefeld (1998) Am. J. Hum. Genet. 62:950-961]. The discordant unaffected sibling provides information on the alleles not segregating to affected individuals.

The FBAT is a unified approach to family-based association testing that is similar in design to the TDT but can accommodate variations in pedigree structures, arbitrary missing genotype information and various different disease models (Rabinowitz and Laird (2000) Hum. Hered. 50:227-233; Laird et al (2000) Genet. Epi. 19 (Suppl. 1):S36-S42). To account for the presence of linkage when using multiple affected siblings per nuclear family, the FBAT allows robust variance estimation (referred to as EV-FBAT) based on the empirical variance-covariance matrix of the contributions of each family to the score statistic (Lake et al. (2000) Am. J. Hum. Genet. 67:1515-1525).

A number of computer software programs are available for statistical analysis of genotyping data in family-based association tests, including the FBAT program [Rabinowitz and Laird (2000) Hum. Hered. 50:211-223; see also www.biostat.harvard.edu/fbat/default.html], the GASSOC program of statistical methods including an extension of the TDT for multiple marker alleles [Schaid (1996) Genet Epidemiol. 13:423-449; see also www.mayo.edu.statgen/gassoc], the Quantitative (Trait) Transmission/Disequilibrium Test (QTDT) which includes support for the methods of Abecasis et al. [(2000) Am. J. Hum. Genet. 66:279-292], Fulker et al. [(1999) Am. J. Hum. Genet. 64:259-267], Monks et al. [(1998) Am. J. Hum. Genet. 63:1507-1516], Allison [(1997) Am. J. Hum. Genet. 60:676-690; TDTQ5] and Rabinowitz [(1997) Hum. Hered. 47:342-350] [see also www.well.ox.ac.uk/asthma/QTDT], the Transmission Disequilibrium Test and SIB Transmission Disequilibrium Test (TDT/S-TDT) [Spielman et al. (1993) Am. J. Hum. Genet. 52:506-516 and Spielman and Ewens (1998) Am. J. Hum. Genet. 62:450-458; see also spielman07.med.upenn.edu/TDT.htm], the ASSOC program in the Statistical Analysis for Genetic Epidemiology (SAGE) program uses the method of George and Elston [(1987) Genet. Epidemiol. 4:193-201; see also darwin.cwru.edu/pub/sage.html] and TRANSMIT [see www.mrc-bsu.cam.ac.uk/pub/methodology/genetics/]. The TRANSMIT program is a modification of the TDT that can handle multilocus haplotypes even if parental genotype or haplotype phase is missing.

The skilled person can carry out association studies with polymorphisms (polymorphic regions) of the IDE, SNCG, KNSL1, LIPA, TNFRSF6 and PLAU genes and surrounding regions. In doing so, significant associations between the polymorphic markers (allelic variants) can form the basis for a variety of methods employing detection and/or use of the polymorphisms or variants, such as, for example, diagnostic, pharmacogenomic and drug-screening methods.

3. Haplotype Analysis

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: the disease mutation is found only in the presence of a specific set of marker alleles. Through subsequent generations, recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are farther away. When not broken up by recombination, “ancestral” haplotypes and linkage disequilibrium between marker alleles at different loci can be tracked not only through pedigrees but also through populations.

A haplotype can be tracked through populations and its statistical association with a given trait can be analyzed. Complementing single point (allelic) association studies with multi-point association studies, also called haplotype studies, increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of markers can be determined. The haplotype frequency is then compared for distinct populations of trait positive and control individuals. The number of trait positive individuals, which should be subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found, the relative risk for an individual carrying the given haplotype of being affected with the trait under study can be approximated.

Detecting an association between a haplotype and a phenotype generally can involve the steps of: a) estimating the frequency of at least one haplotype in a trait positive population; b) estimating the frequency of this haplotype in a control population (trait negative or random population); and c) determining whether a statistically significant association exists between the haplotype and the phenotype. Methods of detecting an association between a haplotype and a phenotype include any method described herein or known in the art.

Determination of Haplotype Frequencies

When genotypes are determined, it is often not possible to distinguish heterozygotes'so that haplotype frequencies cannot be easily inferred. When the gametic phase is not known, single chromosomes can be studied independently, for example, by asymmetric PCR amplification (see Newton et al. (1989) Nucleic Acids Res. 17:2503-2516; Wu et al. (1989) Proc. Natl. Acad. Sci. U.S.A. 86:2757), or by isolation of single chromosomes by limit dilution followed by PCR amplification (see Ruano et al. (1990) Proc. Natl. Acad. Sci. U.S.A. 86:9079-9083). Further, a sample may be haplotyped for sufficiently close markers by double PCR amplification of specific alleles (Sarkar, G. and Sommer S. S. (1991) Biotechniques). These approaches may not be entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G. (1990) Mol. Biol. Evol 7:111-122 may be used. Briefly, the principle is to start filling a preliminary list of haplotypes present in the sample by, examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site.

Alternatively, haplotype frequencies can be estimated from the multilocus genotypic data. Any method known to person skilled in the art can be used to estimate haplotype frequencies (see, e.g., Lange (1997) “Mathematical and Statistical Methods for Genetic Analysis,” (Springer N.Y.); Weir (1996) “Genetic Data Analysis II: Methods for Discrete Population Genetic Data,” Sinauer Assoc., Inc. (Sunderland, Mass. U.S.A.)). For example, maximum likelihood haplotype frequencies can be computed using an Expectation-Maximization (EM) algorithm (see, e.g., Dempster et al. (1977) J. R. Stat. Soc. 39B:1-38; Excoffier and Slatkin (1995) Mol. Biol. Evol. 12:921-927). This procedure is an iterative process aiming at obtaining maximum likelihood estimates of haplotype frequencies from multi-locus genotype data when the gametic phase is unknown. Haplotype estimations are usually performed by applying the EM algorithm using for example the EM-HAPLO program (Hawley et al. (1994) Am. J. Phys. Anthropol. 18:104) or the Arlequin program (Schneider et al. (1997) “Arlequin: A Software for Population Genetics Data Analysis,” Univ. of Geneva). The EM algorithm is a generalized iterative maximum likelihood approach.

To ensure that the estimation finally obtained is the maximum-likelihood estimation, several values of departures are required. The estimations obtained are compared, and, if they are different, the estimations leading to the best likelihood are kept.

Estimating the frequency of a haplotype for a set of polymorphisms in a population can be carried out by: 1) genotyping at least one polymorphism for each individual in a population; 2) genotyping a second polymorphism by determining the identity of the nucleotides at the location of the polymorphism for both copies of the second polymorphism present in the genome of each individual in the population; and c) applying a haplotype determination method to the identities of the nucleotides determined in steps a) and b) to obtain an estimate of the frequency. Methods of estimating the frequency of a haplotype encompass methods used alone or in any combination and all others methods known to those of skill in the art in addition to those described herein.

Exemplary haplotypes useful in the methods provided herein, including methods for determining a predisposition to or occurrence of neurodegenerative disease, such as Alzheimer's disease include polymorphic regions of chromosome 10q. One exemplary haplotype, which includes combinations of polymorphisms that associate with risk for AD, includes polymorphisms or polymorphic regions of the IDE gene corresponding to nucleotides 2456, 3279, 3407 and 42943 of SEQ ID NO:187, or the complementary positions thereof. In one embodiment, the nucleotide in IDE at position 2456 of SEQ ID NO:187 is G, at position 3279 of SEQ ID NO:187 is T, at position 3407 of SEQ ID NO:187 is T, and at position 42943 of SEQ ID NO:187 is T, or the complementary positions thereof. In another embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is T, or the complementary positions thereof. In still a further embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is C, or the complementary positions thereof. In yet another embodiment, the nucleotide in IDE at position 2456 of SEQ ID NO:187 is T, at position 3279 of SEQ ID NO:187 is C, at position 3407 of SEQ ID NO:187 is C, and at position 42943 of SEQ ID NO:187 is C, or the complementary positions thereof. Two of the polymorphisms of this haplotype of 4 SNPs are in linkage disequilibrium: the polymorphisms located at positions corresponding to positions 2456 and 3407 in SEQ ID NO:186 or 187 (positions 121239 and 120288 in SEQ ID NO:484). Therefore, haplotypes of 3 SNPs of this 4-SNP haplotype in which only one of the two SNPs that are in linkage disequilibrium is included among the three SNPs are also provided herein. These 3-SNP haplotypes, particular combinations of which are associated with risk for AD, can be used in any of the methods provided herein, including methods of assessing level of risk for AD. In one 3-SNP haplotype of the IDE gene that associates with risk for AD, the SNPs correspond to nucleotide positions 2456, 3279 and 42943 of SEQ ID NO:186 (positions 121239, 120416 and 80752 of SEQ ID NO:484) and, in a particular embodiment, the nucleotide at each position with respect to the sequence shown in SEQ ID NO:186 is a G, T, T (or the complements thereof in the complementary positions). In another 3-SNP haplotype of the IDE gene that associates with risk for AD, the SNPs correspond to nucleotide positions 3279, 3407 and 42943 of SEQ ID NO:186 (positions 120416, 120288 and 80752 of SEQ ID NO:484) and, in a particular embodiment, the nucleotides at the positions with respect to the sequence shown in SEQ ID NO:186 are T, T and T (or the complements thereof in the complementary positions).

Another haplotype of the IDE gene, which includes combinations of polymorphisms that associate with risk for or protection against AD, includes polymorphisms or polymorphic regions of the IDE gene corresponding to nucleotide positions 122260, 120416, 120288, 80752 and 54795 of SEQ ID NO:484, or the complementary positions thereof. In a particular embodiment of the haplotype that associates with risk for AD, the nucleotides at the 5 positions are G, A, G, A and G, respectively, or the complements thereof in the complementary positions. In a particular embodiment of the haplotype that associates with protection against AD, the nucleotides at the 5 positions are A, A, G, A and G, respectively, or the complements thereof in the complementary positions.

Another exemplary haplotype includes multiple polymorphic regions of the KNSL1 gene corresponding to nucleotides 132370, 1 33355, 147842 and 178981 of SEQ ID NO:484, or the complementary positions thereof. In one embodiment of this haplotype that associates with risk for AD, the nucleotide(s): at position 132370 of SEQ ID NO:484 is A; between positions 133354-133355 of SEQ ID NO:484 is a 6, 7 or 8 base pair poly-T insertion corresponding to -TTTTTT(T)(T)-;.at positions 147842-147845 of SEQ ID NO:484 is the 4 base pair insertion corresponding to -AGTT-; and between positions 178980-178981 of SEQ ID NO:484 is the 5 base pair insertion corresponding to -AATTT-. In particular embodiments, the poly-T insertion can be 6 base pairs corresponding to -TTTTTT-; the poly-T insertion can be 7 base pairs corresponding to -TTTTTTT-; or the poly-T insertion can be 8 base pairs corresponding to -TTTTTTTT-. In a particular embodiment, the polyT insertion is 7 bp (-TTTTTTT-).

Another exemplary haplotype includes miultiple polymorphic regions of the LIPA gene corresponding to nucleotides 1852, 6063 and 7820 of SEQ ID NO:468. In one embodiment, the nucleotide at position 1852 of SEQ ID NO:468 is A, at position 6063 of SEQ ID NO:468 is G, and at position 7820 of SEQ ID NO:468 is C, or the complementary positions thereof. This haplotype can be used in methods provided herein such as methods of determining a level of risk for a neurodegenerative disease, e.g., AD, and in particular detecting possible protection against AD.

Another exemplary haplotype includes polymorphisms of a uPA gene or cDNA corresponding to nucleotide positions 3169, 3947, and 6532 of SEQ ID NO:559 or 560, or complementary positions thereof. In particular embodiments, the nucleotide identities at each of the three positions are as follows: T, C and T, respectively, or the complements thereof. The haplotype may also be described as including polymorphisms of a uPA gene or cDNA corresponding to nucleotide positions 498, 898 and 1512 of SEQ ID NO:561, or complementary positions thereof. In particular embodiments, the nucleotide identities at each of the three positions are as follows: T, C and T, respectively, or the complements thereof.

3. Calculation of Linkage Disequilibrium

Linkage disequilibrium is the non-random association of alleles at two or more loci and represents a powerful tool for mapping genes involved in disease traits (see, e.g., Ajioka et al. (1997) Am. J. Human Genet. 60:1439-1447). Any genetic markers may be used in genetic analysis based on linkage disequilibrium. SNPs, because they are densely spaced in the human genome and can be genotyped in greater numbers than other types of genetic markers (such as RFLP or VNTR markers), are particularly useful in genetic analysis based on linkage disequilibrium. When not broken up by recombination, “ancestral” haplotypes and linkage disequilibrium between marker alleles at different loci can be tracked not only through pedigrees but also through populations. Direct determination of linkage disequilibrium (as opposed to the obtaining of indirect evidence of linkage disequilibrium as is obtained in association analysis of a marker, or haplotype, and a trait) is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine scale mapping of a disease locus, it is useful to have some knowledge of the patterns of linkage disequilibrium that exist between markers in the studied region. The mapping resolution achieved through the analysis of linkage disequilibrium is much higher than that of linkage studies.

Because direct calculation of linkage disequilibrium requires a comparison of two genetic positions, it is generally used to quantify the extent of linkage disequilibrium in a chromosomal region once a single- or multi-locus disease association has been identified. Methods familiar to those who practice the art can be used to calculate linkage disequilibrium.

4. Interaction Analysis

The polymorphisms disclosed herein may also be used to identify patterns of polymorphisms associated with detectable traits resulting from polygenic interactions. The analysis of genetic interaction between alleles at unlinked loci (for example alleles of chromosome 1 0, such as alleles of the uPA, SNCG, IDE, KNSL1, TNFRSF6 and LIPA genes and APOE4 on chromosome 19) requires individual genotyping using the techniques described herein. The analysis of allelic interaction among a selected set of markers (polymorphisms) with appropriate level of statistical significance can be considered as a haplotype analysis. Interaction analysis consists in stratifying the case-control populations with respect to a given haplotype for the first loci and performing a haplotype analysis with the second loci with each subpopulation.

Genotypes and haplotypes of polymorphisms in the uPA, SNCG, IDE, LIPA, TNFRSF6 and KNSL1 genes and surrounding regions, can be analyzed for association with diseases including Alzheimer's disease and other neurodegenerative diseases, and for association with protection against such diseases utilizing the above-described methods. These genotypes and/or haplotypes are useful in diagnosis of susceptibility, determining a level of risk and in pharmacogenomics.

The choice of an allelic variant to be analyzed for association, individually or as part of a collection of allelic variants (a haplotype) can include the use of one or more of the following criteria. An allelic variant to be analyzed for association with disease should show a concentration in affected individuals vs unaffected individuals and for protection should show a concentration in unaffected vs affected individuals. The prevalence of the allele should not be such that it is considered too rare. Preferably, the prevalence should be about 20% or greater. If there is significant linkage disequilibrium among a group of alleles, typically only one of the group will be chosen to analyze for association, as it is assumed that the other alleles will give the same results. Of particular interest analysis of allelic variants that potentially affect gene or protein function; such as those that cause missense mutations, cause a significant change in an amino acid or those that alter a gene regulatory element, e.g., a promoter element.

Furthermore, individual alleles and haplotypes of uPA, SNCG, IDE, LIPA, TNFRSF6 and KNSL1 genes and surrounding regions can also be examined with alleles at unlinked loci, such as APOE4, to provide combinations useful in, for example, diagnosis, determining level of risk and/or pharmacogenomics.

M. Methods of Detecting Polymorphisms in Chromosome 10 and Genes Contained Therein

Provided herein are methods of genotyping or haplotyping a subject or individual. The methods include a step of determining the identity of a nucleotide or sequence of nucleotides, or determining the length of a sequence of nucleotides, at a polymorphic region or site of chromosome 10, such as chromosome 10q in a nucleic acid sample. In particular embodiments, the identity of a nucleotide or sequence of nucleotides, or the length of a sequence of nucleotides at a polymorphic region or site of one or more of the IDE, KNSL1, SNCG, LIPA, TNFRSF6 and PLAU genes and surrounding regions is determined. In particular embodiments, the polymorphic region or site is one or more of the polymorphisms of the IDE, KNSL1, SNCG, LIPA, TNFRSF6 and PLAU genes and surrounding regions specifically described or provided herein, such as with reference to nucleotide positions in specified sequences and also with reference to particular nucleotides or sequences of nucleotides.

Also provided are methods of detecting in a nucleic acid sample, such as a sample containing nucleic acid from a subject or individual, the presence or absence of a polymorphism or allelic variant in chromosome 10, such as in chromosome 10q, and, in particular, in one or more of the IDE, KNSL1, SNCG, LIPA, TNFRSF6 and PLAU genes and surrounding regions. In particular embodiments, the polymorphic region or site is one or more of the polymorphisms of the IDE, KNSL1, SNCG, LIPA, TNFRSF6 and PLAU genes and surrounding regions specifically described or provided herein, such as with reference to nucleotide positions in specified sequences and also with reference to particular nucleotides or sequences of nucleotides.

The methods of genotyping, haplotyping and of detecting the presence or absence of a polymorphism or allelic variant can be used in a number of processes. For example, the genotyping, haplotyping and polymorphism detection methods can be used in methods of identifying polymorphisms associated with a disease or disorder, methods of detecting the polymorphisms associated with a disease or disorder, methods of identifying a region(s) of the human genome containing a disease DNA segment or gene, methods for determining the level of risk for a disease or disorder, methods for determining a predisposition to or the occurrence of a disease or disorder, methods for predicting a response to a treatment for a disease, methods for treating a disease, and methods for confirming a phenotypic diagnosis of a disease.

Any method known in the art can be used to identify a nucleotide, nucleotide sequence or the length of a nucleotide sequence. Many methods are available for detecting specific alleles at human polymorphic loci. The preferred method for detecting a particular polymorphism, depends on the nature of the polymorphism. Several methods of determining the presence or absence of allelic variants of a human gene are described below. Methods that are useful are not limited to those described below, but include all available methods.

1. Nucleic Acid Detection Methods

Generally, these methods are based in sequence-specific polynucleotides, oligonucleotides, probes and primers. Any method known to those of skill in the art for detecting a specific nucleotide within a nucleic acid sequence or for determining the identity of a specific nucleotide in a nucleic acid sequence is applicable to the methods of determining the presence or absence of an allelic variant of these genes on chromosome 10. Such methods include, but are not limited to, techniques utilizing nucleic acid hybridization of sequence-specific probes, nucleic acid sequencing, selective amplification, analysis of restriction enzyme digests of the nucleic acid, cleavage of mismatched heteroduplexes of nucleic acid and probe, alterations of electrophoretic mobility, primer specific extension, oligonucleotide ligation assay and single-stranded conformation polymorphism analysis. In particular, primer extension reactions that specifically terminate by incorporating a dideoxynucleotide are useful for detection. Several such general nucleic acid detection assays are known (see, e.g., U.S. Pat. No. 6,030,778).

Any cell type or tissue may be utilized to obtain nucleic acid samples, e.g., bodily fluid such as blood or saliva, dry samples such as hair or skin.

a. Primer Extension-based Methods

Several primer extension-based methods for determining the identity of a particular nucleotide in a nucleic acid sequence have been reported (see, e.g., PCT Application Nos. PCT/US96/03651 (WO96/29431), PCT/US97/20444 (WO 98/20166), PCT/US97/20194 (WO 98/20019), PCT/US91/00046 (WO91/13075), and U.S. Pat. Nos. 5,547,835, 5,605,798, 5,622,824, 5,691,141, 5,872,003, 5,851,765, 5,856,092, 5,900,481, 6,043,031, 6,133,436 and 6,197,498.) In general, a primer is prepared that specifically hybridizes adjacent to a polymorphic site in a particular nucleic acid molecule. The primer is then extended in the presence of one or more dideoxynucleotides, typically with at least one of the dideoxynucleotides being the complement of the nucleotide that is polymorphic at the site. The primer and/or the dideoxynucleotides may be labeled to facilitate a determination of primer extension and identity of the extended nucleotide.

A preferred method of genotyping or determining the presence of an allelic variant two-dye fluorescence polarization detected single base extension (FP-SBE (12)) on an LJL-Biosystems Criterion Analyst AD (Molecular Devices, Sunnyvale, Calif.). PCR primers are designed to yield products between 200-400 bp in length, and are used at a final concentration of 100-300 nM (Invitrogen Corp., Carlsbad, Calif.) along with Taq polymerase (0.25 U/reaction; Qiagen, Valencia, Calif. and Roche, Indianapolis, Ind.) and dNTPs (2.5 uM/rxn; Amersham-Pharmacia, Piscataway, N.J.). All PCR reactions are performed from ˜10 ng of DNA. General PCR thermo-cycling conditions are as follows: initial denaturation 3 minutes at 94° C., followed by 30-35 cycles of denaturation at 94° C. for 45 seconds, primer-specific annealing temperature (see below) for 45 seconds, and product extension at 72° C. for 1 minute. Final extension at 72° C. for six minutes. PCR products can be visualized on 2% agarose-gels to confirm a single product of the correct size. PCR primers and unincorporated dNTPs can be degraded by adding exonuclease I (Exol, 0.1-0.15 U/reaction; New England Biolabs, Beverly, Mass.) and shrimp alkaline phosphatase (SAP, 1 U/reaction; Roche, Indianapolis, Ind.) to the PCR reactions and incubating for 1 hour at 37° C., followed by 15 minutes at 95° C. to inactivate the enzymes. The single base extension step is performed by directly adding SBE primer (100 nM; Invitrogen Corp., Carlsbad, Calif.), Thermosequenase (0.4 U/reaction; Amersham-Pharmacia, Piscataway, N.J.), and the appropriate mixture of R10-ddNTP, TAMRA-ddNTP (3uM; NEN, Boston, Mass.), and all four unlabeled ddNTPs (22 or 25 uM; Amersham-Pharmacia, Piscataway, N.J.) to the Exol/SAP treated PCR product. Acycloprime-FP SNP detection kits (G/A)(Perkin-Elmer, Boston, Mass.) may also be used for the SBE reaction. Incorporation of the SNP specific fluorescent ddNTP is achieved by subjecting samples to 35 cycles of 94° C. for 15 seconds and 55° C. for 30 seconds. The length of the SBE primers are designed to yield a melting temperature Tₘ of 62-64° C. Fluorescent ddNTP incorporation is detected using the Analyst™ AD System (Molecular Devices, Sunnyvale, Calif.) and measuring fluorescent polarization for R110 (excitation at 490 nm, emission at 520 nm) and TAMRA (excitation at 550 nm, emission at 580 nm). Genotypes are called manually or automatically using the manufacturer's software (‘Allelecaller vers. 1.0’, Molecular Devices, Sunnyvale, Calif.). In view of the polymorphic regions provided herein, SNP specific PCR primers (5′ to 3′ sequences), annealing temperature, product length, SBE primer sequence, SNP location and reference sequence position, can readily be determined by those of skill in the art using well-known methods.

b. Polymorphism-Specific Probe Hybridization

Another detection method is allele specific hybridization using probes overlapping the polymorphic site and having about 5, 10, 15, 20, 25, or 30 nucleotides around the polymorphic region. The probes can contain naturally occurring or modified nucleotides (see U.S. Pat. No. 6,156,501). For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163; Saiki et al. (1989) Proc. Natl Acad. Sci U.S.A. 86:6230; and Wallace et al. (1979) Nucl. Acids Res. 6:3543). Such allele specific oligonucleotide hybridization techniques may be used for the simultaneous detection of several nucleotide changes in different polymorphic regions. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. In a preferred embodiment, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix, Santa Clara, Calif.). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip includes all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

c. Nucleic Acid Amplification-Based Methods

In other detection methods, it is necessary to first amplify at least a portion of a gene prior to identifying the allelic variant. Amplification can be performed, e.g., by PCR and/or LCR, according to methods known in the art. In one embodiment, genomic DNA of a cell is exposed to two PCR primers and amplification is performed for a number of cycles sufficient to produce the required amount of amplified DNA. In another embodiment, the primers are located between 150 and 350 base pairs apart.

Alternative amplification methods include: self sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

Alternatively, allele specific amplification technology, which depends on selective PCR amplification may be used in conjunction with the alleles provided herein. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). In addition it may be desirable to introduce a restriction site in the region of the mutation to create cleavage-based detection (Gasparini et al. (1992) Mol. Cell Probes 6:1).

d. Nucleic Acid Sequencing-Based Methods

Any of a variety of sequencing reactions known in the art can be used to directly sequence at least a portion of a gene and to detect allelic variants, e.g., mutations, by comparing the sequence of the sample sequence with the corresponding wild-type (control) sequence. Exemplary sequencing reactions include those based on techniques developed by Maxam and Gilbert (1977) Proc. Natl. Acad. Sci. U.S.A. 74:560) or Sanger et al. (1977) Proc. Natl. Acad. Sci 74:5463. It is also contemplated that any of a variety of automated sequencing procedures may be used when performing the subject assays ((1995) Biotechniques 19:448), including sequencing by mass spectrometry (see, for example, U.S. Pat. Nos. 5,547,835, 5,691,141, and International PCT Application No. PCT/US94/00193 (WO 94/16101), entitled “DNA Sequencing by Mass Spectrometry” by H. Koster; U.S. Pat. Nos. 5,547,835, 5,622,824, 5,851,765, 5,872,003, 6,074,823, 6,140,053 and International PCT Application No. PCT/US94/02938 (WO 94/21822), entitled “DNA Sequencing by Mass Spectrometry Via Exonuclease Degradation” by H. Koster, and U.S. Pat. Nos. 5,605,798, 6,043,031, 6,197,498, and International Patent Application No. PCT/US96/03651 (WO 96/29431) entitled “DNA Diagnostics Based on Mass Spectrometry” by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are known (see, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”).

e. Restriction Enzyme Digest Analysis

In some cases, the presence of a specific allele in nucleic acid, particularly DNA, from a subject can be shown by restriction enzyme analysis. For example, a specific nucleotide polymorphism can result in a nucleotide sequence containing a restriction site which is absent from the nucleotide sequence of another allelic variant.

f. Mismatch Cleavage

Protection from cleavage agents, such as, but not limited to, a nuclease, hydroxylamine or osmium tetroxide and with piperidine, can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g., RNA or DNA, obtained from a tissue sample. The double-stranded duplexes are treated with an agent, which cleaves single-stranded regions of the duplex such as duplexes formed based on basepair mismatches between the control and sample strands. For instance, RNA/DNA duplexes can be treated with RNase and DNA/DNA hybrids treated with Si nuclease to enzymatically digest the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they differ (see, for example, Cotton et al. (1988) Proc. Natl Acad Sci U.S.A. 85:4397; Saleeba et al. (1992) Methods Enzymod. 217:286-295). The control or sample nucleic acid is labeled for detection.

g. Electrophoretic Mobility Alterations

In other embodiments, alteration in electrophoretic mobility is used to identify the type of allelic variant of a gene of interest. For example, single-strand conformation polymorphism (SSCP) may be used to detect differences in electrophoretic mobility between mutant and wild type nucleic acids (Orita et al. (1989) Proc. Natl. Acad. Sci. U.S.A. 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method uses heteroduplex analysis to separate double stranded heteroduplex molecules on the basis of changes in electrophoretic mobility (Keen et al. (1991) Trends Genet 7:5).

h. Polyacrylamide Gel Electrophoresis

In yet another embodiment, the identity of an allelic variant of a polymorphic region of an gene is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

i. Oligonucleotide Ligation Assay (OLA)

In another embodiment, identification of the allelic variant is carried out using an oligonucleotide ligation assay (OLA), as described, e.g., in U.S. Pat. No. 4,998,617 and in Landegren, U. et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. U.S.A. 87:8923-8927). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucl. Acids Res. 24:3728, OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

j. SNP Detection Methods

Several methods have been developed to facilitate the analysis of single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment, a solution-based method for determining the identity of the nucleotide of a polymorphic site is employed (Cohen, D. et al. (French Patent 2,650,840; PCT Application No. WO/91/02087)). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

k. Genetic Bit Analysis

An alternative method, known as Genetic Bit Analysis or GBAT is described by Goelet, et al. (U.S. Pat. No. 6,004,744, PCT Application No. 92/15712). The method of Goelet, et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO191/02087), the method of Goelet, et al., is preferably a heterogeneous phase assay, in which the primer or the target molecule is immobilized to a solid phase.

I. Other Primer-Guided Nucleotide Incorporation Procedures

Other primer-guided nucleotide incorporation procedures for assaying polymorphic sites in DNA have been described (Komher, J. S. et al. (1989) Nucl. Acids Res. 1-7:7779-7784; Sokolov, B. P. (1990) Nucl. Acids Res. 18:3671; Syvanen, A. C., et al. (1990) Genomics 8:684-692, Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1,992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al. (1993) Amer. J. Hum. Genet. 52:46-59).

For determining the identity of the allelic variant of a polymorphic region located in the coding region of a gene, yet other methods than those described above can be used. For example, identification of an allelic variant which encodes a mutated protein can be performed by using an antibody specifically recognizing the mutant protein in, e.g., immunohistochemistry or immunoprecipitation. Binding assays are known in the art and involve, e.g., obtaining cells from a subject, and performing binding experiments with a labeled lipid, to determine whether binding to the mutated form of the protein differs from binding to the wild-type protein.

m. Molecular Structure Determination

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and single-strand conformation polymorphism.

n. Mass Spectrometric Methods

Nucleic acids can also be analyzed by detection methods and protocols, particularly those that rely on mass spectrometry (see, e.g., U.S. Pat. Nos. 5,605,798, 6,043,031, 6,197,498, and International Patent Application No. WO/96/29431, International PCT Application No. WO/98/20019).

Multiplex methods allow for the simultaneous detection of more than one polymorphic region in a particular gene. This is the preferred method for carrying out haplotype analysis of allelic variants of a gene.

Multiplexing can be achieved by several different methodologies. For example, several mutations can be simultaneously detected on one target sequence by employing corresponding detector (probe) molecules (e.g., oligonucleotides or oligonucleotide mimetics). Variations in additions to those set forth herein will be apparent to the skilled artisan.

A different multiplex detection format is one in which differentiation isaccomplished by employing different specific capture sequences which are position-specifically immobilized on a flat surface (e.g., a ‘chip array’).

o. Other Methods

Additional methods of analyzing nucleic acids include amplification-based methods including polymerase chain reaction (PCR), ligase chain reaction (LCR), mini-PCR, rolling circle amplification, autocatalytic methods, such as those using QJ replicase, TAS, 3SR, and any other suitable method known to those of skill in the art.

Other methods for analysis and identification and detection of polymorphisms, include but are not limited to, allele specific probes, Southern analyses, and other such analyses.

2. Primers, Probes and Antisense Nucleic Acid Molecules

Provided herein are oligonucleotides, such as, for example, primers, probes and antisense nucleic acid molecules. The probes and primers and antisense molecules are oligonucleotides that specifically hybridize to either strand of an IDE, KNSL1, SNCG, LIPA, TNFRSF6 or PLAU gene, or portion thereof, or surrounding regions of chromosome 10, or a nucleic acid molecule comprising a sequence of nucleotides encoding all or one or more portions of a protein encoded by an IDE, KNSL1, SNCG, LIPA, TNFRSF6 or PLAU gene. The probes, primers, and antisense nucleic acids hybridize adjacent to or at a polymorphic region, typically under conditions of moderate or high stringency.

Primers refer to nucleic acids which are capable of specifically hybridizing to a nucleic acid sequence which is adjacent to a polymorphic region of interest, or at a polymorphic region, and are extended. A primer can be used alone in a detection method, or a primer can be used together with at least one other primer or probe in a detection method. Primers can also be used to amplify at least a portion of a nucleic acid. For amplifying at least a portion of a nucleic acid, a forward primer (i.e., 5′ primer) and a reverse primer (i.e., 3′ primer) will preferably be used. Forward and reverse primers hybridize to complementary stands of a double stranded nucleic acid, such that upon extension from each primer, a double stranded nucleic acid is amplified.

Probes refer to nucleic acids which hybridize to the region of interest and which are not further extended. For example, a probe is a nucleic acid which hybridizes adjacent to or at a polymorphic region of a gene of interest on chromosome 10 and which by hybridization or absence of hybridization to the DNA of a subject will be indicative of the identity of the allelic variant of the polymorphic region of the gene. Preferred probes have a number of nucleotides sufficient to allow specific hybridization to the target nucleotide sequence. Where the target nucleotide sequence is present in a large fragment of DNA, such as a genomic DNA fragment of several tens or hundreds of kilobases, the size of a probe may have to be longer to provide sufficiently specific hybridization, as compared to a probe which is used to detect a target sequence which is present in a shorter fragment of DNA. For example, in some diagnostic methods, a portion of a gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

Primers and probes (RNA, DNA (single-stranded or double-stranded), PNA and their analogs) described herein may be labeled with any detectable reporter or signal moiety including, but not limited to radioisotopes, enzymes, antigens, antibodies, spectrophotometric reagents, chemiluminescent reagents, fluorescent and any other light producing chemicals. Additionally, these probes may be modified without changing the substance of their purpose by terminal addition of nucleotides designed to incorporate restriction sites or other useful sequences, proteins, signal generating ligands such as acridinium esters, and/or paramagnetic particles.

These probes may also be modified by the addition of a capture moiety (including, but not limited to para-magnetic particles, biotin, fluorescein, digoxigenin, antigens, antibodies) or attached to the walls of microtiter trays to assist in the solid phase capture and purification of these probes and any DNA or RNA hybridized to these probes. Fluorescein may be used as a signal moiety as well as a capture moiety, the latter by interacting with an anti-fluorescein antibody.

Any probe, primer or antisense molecule can be prepared according to methods well known in the art and described, e.g., in Sambrook, J. Fritsch, E. F., and Maniatis, T. (1989) “Molecular Cloning: A Laboratory Manual,” 2d ed., Cold Spring Harbor Laboratory Press (Cold Spring Harbor, N.Y.) For example, discrete fragments of the DNA can be prepared and cloned using restriction enzymes. Alternatively, probes and primers can be prepared using the Polymerase Chain Reaction (PCR) using primers having an appropriate sequence.

Oligonucleotides may be synthesized by standard methods known in the art, e.g., by use of an automated DNA synthesizer (such as are commercially available from Biosearch (Novato, Calif.); Applied Biosystems (Foster City, Calif.) and other methods). As examples, phosphorothioate oligonucleotides may be synthesized by the method of Stein et al. (1988) Nucl. Acids Res. 16:3209, methylphosphonate oligonucleotides, for example, can be prepared by use of controlled pore glass polymer supports (Sarin et al. (1988) Proc. Natl. Acad. Sci. (U.S.A.) 85:7448-7451).

Suitable primers for the detection of a human polymorphism in these genes can be readily designed using currently available sequence information and standard techniques known in the art for the design and optimization of primers sequences. Optimal design of such primer sequences can be achieved, for example, by the use of commercially available primer selection programs such as Primer 2.1, Primer 3 (www.hgmp.mrc.ac.uk/GenomeWeb/nuc-primer.html) or GeneFisher (genefisher.de/).

Isolated nucleic acids provided herein, which can be used in methods, such as for example the generation of recombinant cells, transgenic animals and agent screening methods, provided herein are generally of a length that provides a sequence that is unique within a genome (e.g., a mammalian, such as human, genome). Antisense nucleic acids, probes and primers provided herein and used, for example, in the methods of detecting allelic variants of a gene of interest are of sufficient length to specifically hybridize to portions of the gene at polymorphic sites. Typically such lengths for the isolated nucleic acids, antisense nucleic acids, probes and primers depend upon the complexity of the source organism genome. For humans such lengths are at least 14, 15, 16, 17, 18 or 19 nucleotides, and typically may be at least 20, 30, 40, 50, 60, 70, 80, 90, 100, 200, 300, 400 or 500 or more nucleotides. In other embodiments, such lengths of the probes and primers provided are not more than 14, 15, 16, 17, 18 or 19 nucleotides, and further may be not more than 20, 30, 40, 50, 60, 70, 80, 90, 100, 200, 300, 400, 500, 600, 700, 800, 900 or 1000 nucleotides in length.

For the methods of detecting polymorphisms in the human SNCG gene provided herein, probes and primers include the following: a sequence of nucleotides that specifically hybridizes adjacent to or at a polymorphic region of a SNCG allele, or the complement thereof, spanning a nucleotide position of SEQ ID NO:73, selected from nucleotide positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200. In a particular embodiment, the nucleotide at position 560 is G or A, at position 590 is A or C, at position 617 is C or T, at position 645 is G or A, at position 915 is T or G, at position 987 is C or A, at position 1723 is A or G, at position 1943 is G or C, at position 1950 is G or A, at position 3151 is A or G, at position 3178 is T or C, at position 3189 is T or C, at position 3284 is G or A, at position 3779 is T or position 3779 is deleted, at position 4156 corresponds to a single nucleotide G that is either inserted or not inserted, at position 4276 is T or A, at position 4311 is C or T, at position 4552 is T or A, at position 4976 is C or position 4976 is deleted, at position 4995 is C or G, at position 5019 is C or T, at position 5025 is C or A, at position 5112 is T or A, at position 5136 is T or A, at position 5517 is T or C, at position 2533 is T or G, at position 3371 is A or C, at position 4627 is T or G, at position 4727 is A or G. at position 4813 is A or C, and at position 5200 is G or C.

For the methods of detecting polymorphisms in the human IDE gene provided herein, probes and primers include the following: a sequence of nucleotides that specifically hybridizes adjacent to or at a polymorphic region of an IDE allele, or the complement thereof, spanning a nucleotide position of SEQ ID NO:187 selected from nucleotide positions 2456, 3279, 3407, 42943, 62498, 69586, 107395, 112114, 116662, 17095, 17242, 33590, 38903, 43391, 45017, 68906, 68973, 73772, 74084, 83024, 83104, 89301, 105060, 108489, 111914, 113142, 113591, 114683, 117803 and 124565; or and IDE allele spanning a nucleotide position of SEQ ID NO:484, or the complement thereof, selected from the group consisting of nucleotide positions 820, 7066,11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444. In a particular embodiment, the nucleotide in SEQ ID NO:187 at position 2456 is T or G, at position 3279 is T or C, at position 3407 is C or T, at position 42943 is T or C, at position 62498 is T or C, at position 69586 is T or C, at position 107395 is G or A, at position 112114 is G or A, and at position 116662 is T or A and/or the complementary nucleotide(s) in SEQ ID NO:484: at position 820 is A or T, at position 7066 is A or G, at position 11758 is T or C, at position 21270 is T or G, at position 22225 is A or T, at position 29294 is C or T, at position 33452 is G or T, at position 33708 is G or A, at position 36982 is C or T, at position 54862 is A or G, at position 77786 is C or A, at position 80594 is G or A, at position 84792 is T or C, at position 84997 is G or T, at position 86682 is C or T, at position 86857 is T or A, at position 88511 is A or G, at position 90437 is G or T, at position 90593 is G or A, at position 91650 is T or C, at position 91870 is G or A, at position 91878 is G or A, at position 92011 is C or T, at position 93618 is T or C, at position 94344 is C or T, at position 94714 is A or G, at position 95671 is A or G, at position 96324 is A or G, at position 97302 is G or A, at position 97370 is G or A, at position 98253 is T or C, at position 98276 is C or T, at position 98385 is A or G, at position 98646 is T or A, at position 98814 is G or A, at position 99597 is C or T, at position 100378 is T or C, at position 101029 is G or A, at position 101265 is C or T, at position 102465 is C or G, at position 103289 is T or G, at position 103967 is C or T, at position 105793 is A or G, at position 106076 is G or T, at position 106453 is C or T, at position 106600 is A or G, at position 106995 is G or A, at position 107851 is C or T, at position 108434 is G or C, at position 109096 is C or T, at position 109399 is C or T, at position 109483 is T or G, at position 110870 is G or A, at position 1111 89 is A or G, at position 111972 is G or A, at position 112627 is A or T, at position 112629 is A or T, at position 112631 is T or A, at position 11 3407 is C or G, at position 114444 is C or G, at position 114482 is G or C, at position 115473 is C or position 115473 is deleted, at position 116681 is G or T, at position 117226 is A or T, at position 117600 is A or G, at position 1l 7802 is C or T, at position 11 8223 is G or C, at position 120011 is C or T, at position 122260 is A or G, at position 123165 is A or G, at position 123424 is G or A, at position 124352 is A or G, at position 124501 is C or T, at position 124692 is A or G, at position 125113 is T or A, at position 125159 is G or A, at position 126568 is G or C, at position 127166 is C or G, at position 127598 is T or C, at position 127600 is T or C, at position 127609 is T or C, at position 127614 is T or C, at position 127623 is T or C, at position 127662 is G or A, at position 128053 is G or A, at position 128261 is a repeat of -TAAA- occurring 6, 7, or 8 times beginning at position 128261, at position 128289 is A or T, at position 128291 is T or G, at position 128393 is T or G, at position 129444 is C or T.

For the methods of detecting polymorphisms in the human KNSL1 gene provided herein, probes and primers include the following: a sequence of nucleotides that specifically hybridizes adjacent to or at a polymorphic region of a KNSL1 allele, or the complement thereof, spanning a nucleotide position of SEQ ID NO:348 selected from nucleotide positions 300, 1152, 14235, 15104, 20815, 35719, 36738-36739, 41015, 42125, 45083, 45887, 56706, 56887, 58524, 62661 and 63802; or a KNSL1 allele spanning a nucleotide position of SEQ ID NO:484, or the complement thereof, selected from the group consisting of nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706. In a particular embodiment, the nucleotide(s) at position 300 corresponds to a dinucleotide -CA- that is either inserted or not inserted beginning at position 300, at position 1152 is G or T, at position 14235 corresponds to a single nucleotide T that is either inserted or not inserted, at position 15104 is A or G, at position 20815 is T or C, at position 35719 is T or C, at positions 36738-36739 is a dinucleotide corresponding to CA or AC, at position 41015 corresponds to the oligonucleotide -AATTT- that is either inserted or not inserted beginning at position 41015, at position 42125 is T or G, at position 45083 is C or T, at position 45887 is G or C, at position 56706 is C or T, at position 56887 is A or G, at position 58524 is C or T, at position 62661 is C or T, and at position 63802 is A or C; and/or the nucleotide(s) in SEQ ID NO:484: at position 130876 is T or C, at position 131378 is G or A, at position 131616 is G or A, at position 131620 is G or A, at position 131688 is T or G, at positions 131998-131203 are CTTTTC- or positions 131998-131203 are deleted, at position 132004 is either a 9, 16, 21, 26, or 29 base pair poly-T repeat beginning at nucleotide 1 32004, at position 1 32370 is A or G, at position 132697 is A or G, at position 132968 is C or T, at position 133355 is either a 6, 7 or 8 base pair poly-T repeat beginning at nucleotide 133355, at position 133806 is T or G, at position 134030 is G or A, at position 134291 is A or G, at position 134661 is G or A, at position 137087 is A or G, at position 137142 is G or A, at position 138396 is C or T, at position 140665 is T or G, at position 140736 is A or G, at position 141173 is A or G, at position 142056 is T or C, at position 142777 corresponds to a dinucleotide -AG- that is either inserted or not inserted beginning at position 142777, at position 143025 is G or T, at position 143729 is C or A, at position 144484 is T or A, at position 146181 is T or A, at position 147051 is G or A, at position 147322 is C or T, at position 147707 is G or T, at positions 147842-147845 are -AGTT- or positions 147842-147845 are deleted, at position 148080 is C or T, at position 149026 is either a 17, 18, 19 or 22 base pair -AC- repeat beginning at nucleotide 149026, at position 149044 is either a 22, 24, 28, 30, 32 or 36 base pair -GT- repeat beginning at nucleotide 149044, at position 149389 is A or G, at position 150003 is G or A, at position 150384 is G or T, at position 150454 is C or T, at position 150686 is G or T, at position 151343 is C or T, at position 151961 is C or T, at position 152119 is C or T, at position 153791 is C or G, at position 154328 is A or T, at position 154513 is C or A, at position 154639 is G or A, at position 155049 is T or C, at position 155114 is T or C, at position 158040 is C or A, at position 158895 is G or A, at position. 191284 is C or T, at position 192272 is C or T, at position 192698 is A or T, at position 193706 is T or A.

For the methods of detecting polymorphisms in the human TNFRSF6 genes provided herein, probes and primers include the following: a sequence of nucleotides that specifically hybridizes adjacent to or at a polymorphic region of a TNFRSF6 allele or the complement thereof spanning a position corresponding to a position of SEQ ID NO:403 selected from the group consisting of positions 1530, 1550, 14525, 14714, 18982, 19069, 20412, 20552, 23199, 23416, 24890, 26359, 199, 213, 843, 2967, 3103, 5335,,5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026. In a particular embodiment, the nucleotide at position 1530 is T or C, at position 1550 is A or G, at position 14525 is G or A, at position 14714 is C or T, at position 1 8982 is G or C, at position 19069 is A or G, at position 20412 is A or G, at position 20552 is A or G, at position 23199 is G or A, at position 23416 is T or C, at position 24890 is A or G, at position 26359 is A or T, at position 1926 is G or A, at position 2269 is G or A, at position 1 8934 is C or T, at position 19227 is C or T, and at position 22026 is C or G.

For the methods of detecting polymorphisms in the human LIPA genes provided herein, probes and primers include the following: a sequence of nucleotides that specifically hybridizes adjacent to or at a polymorphic region of a LIPA allele or the complement thereof spanning a position corresponding to a position of SEQ ID NO:468 selected from the group consisting of positions 1197, 1307-1309, 1841, 1852, 2075, 6063, 6173, 6194, 7820, 25283, 28453-28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969. In a particular embodiment, the nucleotide at position 1197 is C or G, at positions 1307-1309 are ATC or positions 1307-1309 are deleted, at position 1841 is A or C, at position 1852 is G or A, at position 2075 is G or A, at position 6063 is G or T, at position 6173 is A or C, at position 6194 is G or A, at position 7820 is C or G, at position 25283 is G or C, at positions 28453-28465 are -TCCGCGAGAGGGC- or positions 28453-28465 are deleted, at position 28543 is C or T, at position 28746 is A or C, at position 29904 is G or A, at position 37861 is C or T, at position 39834 is T or A, and at position 40018 is C or T.

For the methods of detecting polymorphisms in the human uPA (PLAU) gene provided herein, probes and primers include the following: a sequence of nucleotides that specifically hybridizes adjacent to or at a polymorphic region of a uPA allele or the complement thereof spanning a position corresponding to a position of SEQ ID NO:559 or 560 selected from the group consisting of positions 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848, 7908, and the complementary positions thereof; and positions of SEQ ID NO:563 consisting of 79, 93, 256, 385 and 714, and the complementary positions thereof. In particular embodiments, the nucleotide(s) in SEQ ID NO:559 or 560: at position 9 is A or C, at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, and at position 6532 is T or C, and the complements thereof; and the nucleotide in SEQ ID NO:563: at position 79 is T or C, at position 93 is a C or position 93 is deleted, at position 256 is G or T, at position 385 is C or T, at position 714-715 is the dinucleotide -GT- or the -GT- dinucleotide is deleted.

Antisense compounds may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Antisense compounds are typically 8 to 30 nucleotides in length complementary to and targeted to a nucleic acid molecule and modulates its expression. The targeted nucleic acid molecule represents the coding strand. For example, an antisense compound is an antisense oligonucleotide which comprises the complement of at least an 8 nucleotide segment of the SNCG gene (SEQ ID NO:73) or RNA (SEQ ID NO:469); an 8 nucleotide segment of the IDE gene (SEQ ID NO:187) or RNA (SEQ ID NO:470); an 8 nucleotide segment of the KNSL1 gene (SEQ ID NO:348) or RNA (SEQ ID NQ:471, SEQ ID NO:473 or SEQ ID NO:475); an 8 nucleotide segment of the TNFRSF6 gene (SEQ ID NO:403) or RNA (SEQ ID NO:477 through SEQ ID NO:481); an 8 nucleotide segment of the LIPA gene (SEQ ID NO:468) or RNA (SEQ ID NO:482).

In a particular embodiment, antisense compounds provided herein, comprise the complement of at least an 8 nucleotide segment of a cDNA encoding a polymorphic SNCG protein comprising the coding region or full-length of SEQ ID NO:469 having variant nucleotides corresponding to positions: 30, 57, 85, 243, 250, 377, 512, 531, 555, 561 and 672 of SEQ ID NQ:469. In a particular embodiment, an antisense compound provided herein comprises the complement of at least an 8 nucleotide segment of SEQ ID NO:469, wherein the nucleotide at position 672 of SEQ ID NO:469 is not T. In another embodiment, the nucleotide at position 672 of SEQ ID NO:469 is A.

Also provided herein are antisense compounds comprising the complement of at least an 8 nucleotide segment of cDNAs encoding IDE protein comprising the coding region or full-length of SEQ ID NO:470 having a variant nucleotide corresponding to position 7 of SEQ ID NO:470, wherein the nucleotide at position 7 is not C. In a particular embodiment, the nucleotide at position 7 of SEQ ID NO:470 is T.

Also provided herein are antisense compounds comprising the complement of at least an 8 nucleotide segment of cDNAs encoding a polymorphic KNSL1 protein comprising the coding region or full-length of: SEQ ID NO:471 having a variant nucleotide at position 2747 of SEQ ID NO:471; SEQ ID NO:473 having a variant nucleotide at position 2610 of SEQ ID NO:473; SEQ ID NO:475 having a variant nucleotide at position 2695 of SEQ ID NO:475, wherein the variant nucleotide at each of these positions is not C. In a particular embodiment, the nucleotide at position 2747 of SEQ ID NO:471, at position 2610 of SEQ ID NO:473, and at position 2695 of SEQ ID NO:475 is T.

Also provided herein are antisense compounds comprising the complement of at least an 8 nucleotide segment of cDNAs encoding a polymorphic TNFRSF6 protein comprising the coding region or full-length of SEQ ID NO:477 having variant nucleotides corresponding to positions 208 and 420 of SEQ ID NO:477. In a particular embodiment, an antisense compound provided herein comprises the complement of at least an 8 nucleotide segment of SEQ ID NO:477, wherein the nucleotide at position 208 is not G. In another embodiment, the nucleotide at position 208 of SEQ ID NO:477 is A.

Also provide herein are antisense compounds comprising the complement of at least an 8 nucleotide segment of cDNAs encoding TNFRSF6 protein comprising the coding region or full-length of SEQ ID NO:478 having variant nucleotides corresponding to positions 377, 416, 836 and 1766 of SEQ ID NQ:478. In a particular embodiment, an antisense compound provided herein comprises the complement of at least an 8 nucleotide segment of SEQ ID NO:478, wherein the nucleotide at position 377 is not G. In another embodiment, the nucleotide at position 377 of SEQ ID NO:478 is A.

Also provide herein are antisense compounds comprising the complement of at least an 8 nucleotide segment of cDNAs encoding TNFRSF6 protein comprising the coding region or full-length of SEQ ID NO:479 having variant nucleotides corresponding to positions 403, 442, 862 and 1792 of SEQ ID NO:479. In a particular embodiment, an antisense compound provided herein comprises the complement of at least an 8 nucleotide segment of SEQ ID NO:479, wherein the nucleotide at position 403 is not G. In another embodiment, the nucleotide at position 403 of SEQ ID NO:479 is A.

Also provide herein are antisense compounds comprising the complement of at least an 8 nucleotide segment of cDNAs encoding TNFRSF6 protein comprising the coding region or full-length of SEQ ID NO:480 having variant nucleotides corresponding to positions 208, 247 and 604 of SEQ ID NO:480. In a particular embodiment, an antisense compound provided herein comprises the complement of at least an 8 nucleotide segment of SEQ ID NO:480, wherein the nucleotide at position 208 is not G. In another embodiment, the nucleotide at position 208 of SEQ ID N0:480 is A.

Also provide herein are antisense compounds comprising the complement of at least an 8 nucleotide segment of cDNAs encoding TNFRSF6 protein comprising the coding region or full-length of SEQ ID NO:481 having variant nucleotides corresponding to positions 208 and 247 of SEQ ID N0:481. In a particular embodiment, an antisense compound provided herein comprises the complement of at least an 8 nucleotide segment of SEQ ID NO:481, wherein the nucleotide at position 208 is not G. In another embodiment, the nucleotide at position 208 of SEQ ID NO:481 is A.

Also provided herein are antisense compounds comprising the complement of at least an 8 nucleotide segment of cDNAs encoding a polymorphic LIPA protein comprising the coding region or full-length of SEQ ID NO:482 having variant nucleotides corresponding to positions: 86, 107, 2149, and 2333 of SEQ ID NO:482. In a particular embodiment, an antisense compound provided herein comprises the complement of at least an 8 nucleotide segment of SEQ ID NQ:482, wherein the nucleotide at position 2333 of SEQ ID NO:482 is not C. In another embodiment, the nucleotide at position 2333 of SEQ ID NO:482 is T.

An antisense compound can contain at least one modified nucleotide which can confer nuclease resistance or increase the binding of the antisense compound with the target nucleotide. The antisense compound can contain at least one internucleoside linkage wherein the modified internucleoside linkage of the antisense oligonucleotide can be a phosphorothioate linkage, a morpholino linkage or a peptide-nucleic acid linkage. Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

An antisense compound can contain at least one least one modified sugar moiety wherein the modified sugar moiety of the antisense oligonucleotide is a 2′-O-methoxyethyl sugar moiety or a 2′-dimethylaminooxyethoxy sugar moiety. Modified oligonucleotides may also contain one or more substituted sugar moieties. Representative United States patents that teach the reparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference.

An antisense compound can contain at least one modified nucleobase. Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine, deazaguanine and 7-deazaadenine and 3-deazaguanine and 3-deazaadenine.

Further nucleobases include those disclosed in U.S. Pat. No. 3,687,808, those disclosed in Kroschwitz, J.(1990) “The Concise Encyclopedia Of Polymer Science And Engineering,” John Wiley & Sons 1 ed. 858-859, those disclosed by Englisch et al. (1991) Angewandte Chemie, I ed. 30:613, and those disclosed by Sanghvi, Y. S., Crooke, S. T., and Lebleu, (1993) “Antisense Research and Applications,” CRC Press, B. eds. 289-302 (Boca Raton). Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu (1993) “Antisense Research and Applications,” CRC Press, B. eds. 276-278 (Boca Raton)) and are presently preferred base substitutions, even more particularly when combined with 2′-O-methoxyethyl sugar modifications.

The antisense compound can be a chimeric oligonucleotide. Chimeric antisense compounds may be formed as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

N. Transgenic Animals

Provided herein are transgenic animals, and in particular, non-human transgenic animals, containing, as at least one transgenic element, a uPA, SNCG, IDE, KNSL1, TNFRSF6 and LIPA gene or a portion or portions thereof, such as, for example, a transcriptional control region (including, for example, a promoter and 3′ untranslated (UTR) sequences) and/or a coding sequence of a uPA, SNCG, IDE, KNSL1, TNFRSF6 and LIPA gene. The uPA, SNCG, IDE, KNSL1, TNFRSF6 and LIPA gene or portion(s) thereof contains at least one polymorphic region and is thus referred to as a polymorphic uPA, SNCG, IDE, KNSL1, TNFRSF6 and LIPA gene or portion(s) thereof. A “uPA, SNCG, IDE, KNSL1, TNFRSF6 and LIPA gene or a portion or portions thereof” includes a uPA, SNCG, IDE, KNSL1, TNFRSF6 and LIPA cDNA or portion(s) thereof. In particular embodiments, the polymorphic uPA, SNCG, IDE, KNSL1, TNFRSF6 and LIPA gene is a human polymorphic uPA, SNCG, IDE, KNSL1, TNFRSF6 and LIPA gene. In further particular embodiments, the transgenic animal is a mammal, including, but not limited to rabbits, guinea pigs, cows, pigs, goats, sheep, horses, non-human primates (e.g., baboons, monkeys and chimpanzees) and particularly rodents, including rats and mice. In other embodiments, the animal is an insect, such as, for example, Drosophila. Transgenic animals provided herein may be used for numerous purposes. For example, the animals may be used in testing polymorphic uPA, SNCG, IDE, KNSL1, TNFRSF6 and LIPA genes or portion(s) thereof for characterization of phenotypic outcomes correlated with the particular polymorphisms. The transgenic animals may be used as models for disorders and diseases that involve altered uPA, SNCG, IDE, KNSL1, TNFRSF6 and LIPA gene and/or protein expression or function. Transgenic rodents, such as mice, are particularly well-suited for use as disease models. Transgenic animals containing polymorphic uPA, SNCG, IDE, KNSL1, TNFRSF6 and LIPA genes or portion(s) thereof may also be used in methods of identifying agents that modulate uPA, SNCG, IDE, KNSL1, TNFRSF6 and LIPA expression and/or activity or that modulate a biological event characteristic of a disease or disorder involving altered uPA, SNCG, IDE, KNSL1, TNFRSF6 and LIPA gene and/or protein expression or function which may be candidate treatments for the disease or disorder.

Also provided herein are methods of producing transgenic animals by introducing a polymorphic uPA, SNCG, IDE, KNSL1, TNFRSF6 and LIPA gene or portion(s) thereof into a cell and allowing the cell to develop into a transgenic animal. The cell may be any cell that may be used in the generation of a transgenic animal. Such cells are known to those of skill in the art of transgenic animal production. For example, the cell may be an embryo, zygote, oocyte, fertilized oocyte or embryonic stem cell, such as, for example, a mouse embryonic stem cell. Numerous techniques for introduction of exogenous nucleic acids into cells that will be allowed to develop into transgenic animals are also known to those of skill in the art. Such techniques include, but are not limited to, pronuclear microinjection (see, e.g., U.S. Pat. No. 4,873,191), retrovirus-mediated gene transfer into germ lines [see, e.g., Van der Putten et al. (1985) Proc. Natl. Acad. Sci. U.S.A. 82:6148-6152], gene targeting into embryonic stem cells [see, e.g., Thompson et al. (1989) Cell 56:313-3211, electroporation of embryos [see, e.g., Lo (1983) Mol. Cell. Biol. 3:1803-1814], and sperm-mediated gene transfer [see, e.g., Lavitrano et al. (1989) Cell 57:717-7231 [for a review of such techniques, see Gordon (1989) Int. Rev. Cytol. 115:171-229]. A cell into which exogenous nucleic acid has been transferred may be introduced into a recipient female animal for development into a transgenic animal containing the exogenous nucleic acid.

Methods for making transgenic animals using a variety of transgenes have been described [see, e.g., Wagner et al. (1981) Proc. Nat. Acad. Sc. U.S.A. 78:5016; Stewart et al. (1982) Science 217:1046; Constantini et al. (1981) Nature 294:92; Lacy et al. (1983) Cell 34:343; McKnight et al. (1983) Cell 34:335; Brinstar et al. (1983) Nature 306:332; Palmiter et al. (1982) Nature 300:611; Palmiter et al. (1982) Cell 29:701, and Palmiter et al. (1983) Science 222:809; Ono et al. (2001) Reproduction 122:731-736; Reggio et al. (2001) Biol. Reprod. 65:1528-1533; Park et al. (2001) Animal Reprod. Sci. 68:111-120; Zakhartchenko et al. (2001) Mol. Reprod. Dev. 60:362-369; Arat et al. (2001) Mol. Reprod. Dev. 60:20-26; Koo et al. (2001) Mol. Reprod. Dev. 58:15-20; Polejaeva and Campbell (2000) Theriogenology 53:117-126]. Such methods are also described in U.S. Pat. Nos. 6,175,057; 6,180,849 and 6,133,502, 6,271,436, 6,258,998, 6,103,523, 6,252,133. The term “transgene” is used herein to describe genetic material that has been or is about to be artificially inserted into a cell, particularly an animal cell. For example, the cell may be a mammalian cell, and may be a cell of a living animal. The transgene is used to transform a cell, meaning that a permanent or transient genetic change, is induced in a cell following incorporation of exogenous nucleic acid. A permanent genetic change may be achieved, for example, by introduction of the nucleic acid into the genome of the cell. Vectors for stable integration include, but are not limited to, plasmids, retroviruses and other animal viruses and YACS. Transgenic animals contain an exogenous nucleic acid sequence present as an extrachromosomal element or stably integrated in all or a portion of their cells, especially germ cells. In particular embodiments of the transgenic animals provided herein, the animal stably contains exogenous nucleic acid, e.g., a transgene, in its germ cells for transmission through the germline.

A transgenic animal that contains a transgene in only some, but not all, of its cells is generally referred to as “chimeric.” During the initial construction of a transgenic animal, “chimeras” or “chimeric animals” may be generated. Chimeras are primarily used for breeding purposes in order to generate the desired transgenic animal. Transgenic animals having a heterozygous alteration are generated by breeding of chimeras. Male and female heterozygotes are typically bred to generate homozygous animals.

In general methods of generating a transgenic animal, the exogenous nucleic acid, e.g., transgene, is usually either from a different species than the animal host, or is altered in its coding or non-coding sequence relative to a wild-type or reference nucleic acid. The introduced nucleic acid may be a wild-type gene or portion(s) thereof, a naturally occurring polymorphism or a genetically manipulated sequence, for example having deletions, substitutions or insertions in the coding or non-coding regions. When the introduced nucleic acid contains a coding sequence, it is usually operably linked to a promoter, which may be constitutive or inducible, and other regulatory sequences that may be required for expression in the host animal.

Nucleic acids for use in generating transgenic animals provided herein are nucleic acids containing one or more polymorphic regions of a uPA, SNCG, IDE, KNSL1, TNFRSF6 and LIPA gene and particular polymorphisms thereof, such as particular uPA, SNCG, IDE, KNSL1, TNFRSF6 and LIPA gene alleles. In particular embodiments, the nucleic acid used in generating a transgenic animal contains a human uPA, SNCG, IDE, KNSL1, TNFRSF6 and LIPA gene or portion(s) thereof containing at least one polymorphic region. Of particular interest are variants of a uPA, SNCG, IDE, KNSL1, TNFRSF6 and LIPA gene or portion(s) thereof that are associated, individually and/or in combination, with a disease or disorder, such as a uPA, SNCG, IDE, KNSL1, TNFRSF6 and LIPA-mediated disease or disorder. In particular embodiments of the transgenic animals provided herein, the animal contains a polymorphic uPA, SNCG, IDE, KNSL1, TNFRSF6 and LIPA gene or portion(s) thereof associated, individually and/or in combination, with thrombosis, thrombolytic diseases, stroke, atherosclerosis, coronary artery disease, cardiovascular disease, cardiac disorders, myocardial infarction, cardiomyopathies, proliferative diseases, cancer, tumor angiogenesis, tumor metastasis, arthritis, rheumatic diseases and inflammatory joint diseases. In further embodiments of the transgenic animals provided herein, the animal contains a polymorphic uPA, SNCG, IDE, KNSL1, TNFRSF6 and LIPA gene or portion(s) thereof associated, individually and/or in combination, with a neurodegenerative disease or disorder. In yet further embodiments of the transgenic animals provided herein, the animal contains a polymorphic uPA, SNCG, IDE, KNSL1, TNFRSF6 and LIPA gene or portion(s) thereof associated, individually and/or in combination, with Alzheimer's disease.

Exemplary polymorphic regions and particular allelic variants for use in the transgenic animals provided herein include those set forth in the Examples at Tables 2, 4 and 4-B, 6 and 6-B, 8, 10, 12 and 12-B, and A-F. In a particular embodiment, a transgenic animal provided herein contains heterologous transgenic element nucleic acid comprising a uPA, SNCG, IDE, KNSL1, TNFRSF6 or LIPA nucleic acid molecule described herein in the “Nucleic Acid Molecules”, “Primers, Probes and Antisense Nucleic Acids” and/or the “cDNAs” sections set forth herein. In another embodiment, a transgenic animal provided herein contains heterologous transgenic element nucleic acid containing a uPA, SNCG, IDE, KNSL1, TNFRSF6 or LIPA gene or portion(s) thereof that includes one or more polymorphisms associated individually and/or in combination with a uPA, SNCG, IDE, KNSL1, TNFRSF6 or LIPA-mediated disease or disorder. In a further embodiment, a transgenic animal provided herein contains heterologous transgenic element nucleic acid containing a uPA, SNCG, IDE, KNSL1, TNFRSF6 or LIPA gene or portion(s) thereof that includes one or more polymorphisms associated individually and/or in combination with a neurodegenerative disease or disorder. In particular embodiments, the neurodegenerative disease is Alzheimer's disease. In a yet further embodiment, the neurodegenerative disease is Alzheimer's disease with an age of onset that is greater than or equal to about 50 years, or greater than or equal to about 60 years, or greater than or equal to about 65 years.

In a particular exemplary embodiment, a transgenic animal provided herein contains as a heterologous transgenic element nucleic acid containing a uPA gene or portion(S) thereof that includes one or more polymorphisms that occur at nucleotide positions corresponding to the following nucleotide positions in SEQ ID NO: 559 or 560, or the complementary positions thereof: nucleotide 401 which is an A, T or C; nucleotide 515 which is a T, G or A; nucleotide 748 which is a T, C or A; and nucleotide 1752 which is a C, G or A. In a further particular embodiment, the heterologous transgenic element is nucleic acid containing a uPA gene or portion(s) thereof that includes one or more polymorphisms that occur at nucleotide positions corresponding to the following nucleotide positions in SEQ ID NO: 559 or 560, or the complementary positions thereof: nucleotide 401 which is an A; nucleotide 515 which is a T; nucleotide 748 which is a T; and nucleotide 1752 which is a C, or the complements thereof.

In another embodiment, a transgenic animal provided herein contains as a heterologous transgenic element nucleic acid containing a uPA gene or portion(s) thereof that includes one or more polymorphisms associated individually and/or in combination with a uPA-mediated disease or disorder and that occur at nucleotide positions corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 in SEQ ID NO:563, or the complementary positions thereof.

In a particular embodiment, the transgenic animal contains as a heterologous transgenic element nucleic acid containing a uPA gene or portion(s) thereof that includes one or more polymorphisms associated with a uPA-mediated disease or disorder in combination with one or more polymorphisms that occur at nucleotide positions corresponding to the following nucleotide positions in SEQ ID NO: 559 or 560, or the complementary positions thereof: nucleotide 401 which is an A, T or C, and in particular an A; nucleotide 515 which is a T, G or A, and in particular a T; nucleotide 748 which is a T, C or A, and in particular a T; and nucleotide 1752 which is a C, G or A, and in particular a C. In a further embodiment, the one or more polymorphisms associated with a uPA-mediated disease or disorder in combination with one or more of these polymorphisms at nucleotides corresponding to positions 401, 515, 748 and 1752 of SEQ ID NO: 559 or 560 occur at nucleotide positions corresponding to the following nucleotide positions: nucleotide 464, 1229, 1356, 1942, 2127, 2543, 3029,3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 in SEQ ID NO:563, or the complementary positions thereof.

In another embodiment, a transgenic animal provided herein contains as a heterologous transgenic element nucleic acid containing a uPA gene or portion(s) thereof that includes one or more polymorphisms that is associated individually and/or in combination with a neurodegenerative disease or disorder and that occur at nucleotide positions corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 in SEQ ID NO:563, or the complementary positions thereof. In a particular embodiment, the neurodegenerative disease is Alzheimer's disease. In a yet further embodiment, the neurodegenerative disease is Alzheimer's disease with an age of onset that is greater than or equal to about 50 years, or greater than or equal to about 60 years, or greater than or equal to about 65 years.

In a further embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 9, 401, 464, 515, 748, 1229, 1356, 1752, 1942, 2127, 2543, 3029, 3169, 3799, 3947, 4808, 5287, and 6532, or the complementary positions thereof. In a yet further embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 9, 401, 464, 515, 748, 1229, 1356, 1752, 1942, 2127, 2543, 3029 and 5287, or the complementary positions thereof. In another embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 3169, 3947 and 6532, or the complementary positions thereof. In a particular embodiment, the nucleotide in position 3169 is T, at position 3947 is C, and at position 6532 is T, or the complements thereof. In another embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions: 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotide positions 79, 93, 256, 385 and 714 of SEQ ID NO:563, or the complementary positions thereof. In yet another embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions: 178, 401, 464, 515 and 748 in SEQ ID NO: 559 or 560 and positions 79, 93, 256, 385 and 714 of SEQ ID NO:563, or the complementary positions thereof. In a further embodiment, a transgenic animal provided herein contains as a heterologous transgenic element nucleic acid containing a uPA gene or portion(s) thereof that includes one or mote polymorphisms associated individually and/or in combination with a neurodegenerative disease or disorder and that occur at nucleotide positions corresponding to the following nucleotide positions of SEQ ID NO: 559 or 560: 401, 515 and 748, or the complementary positions thereof. In a further embodiment, a transgenic animal provided herein contains as a heterologous transgenic element nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 6519, 6532, 6909 and 7235, or the complementary positions thereof. In particular embodiments, the neurodegenerative disease is Alzheimer's disease. In a yet further embodiment, the neurodegenerative disease is Alzheimer's disease with an age of onset that is greater than or equal to about 50 years, or greater than or equal to about 60 years, or greater than or equal to about 65 years.

In particular embodiments of any of the above embodiments of the transgenic animals provided herein, the nucleotide at position 9 is A or C, at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, at position 6532 is T or C, at position 178 is A or G, at position 1363 is C or A, at position 1423 is G or T, at position 1465 is C or A, at position 1540 is C or T, at position 2297 is C or T, at position 2445 is T or G, at position 2653 is G or A, at position 3080 is G or A, at position 3546 is C or G, at position 3664 is C or T, at position 3816 is A or C, at position 4320 is T or C, at position 4369 is G or A, at position 4399 is C or A, at position 4851 is G or A, at position 5186 is G or A, at position 5204 is G or A, at position 5787 is C or G, at position 6519 is C or G, at position 6909 is G or T, at position 7235 is G or position 7235 is deleted, at position 7848 is C or T, at position 7908 is A or C; and the nucleotide in SEQ ID NO:563: at position 79 is T or C, at position 93 is a C or position 93 is deleted, at position 256 is G or T, at position 385 is C or T, at position 714-715 is the dinulceotide -GT- or the -GT- dinucleotide is deleted, or the complements thereof.

Transgenic animals can be generated that carry the polymorphic uPA, SNCG, IDE, KNSL1, TNFRSF6 and LIPA gene or a portion(s) thereof in all their cells or in only some of their cells [Lasko et al. (1992) Proc. Natl. Acad. Sci. U.S.A. 89:6232-6236]. A polymorphic uPA, SNCG, IDE, KNSL1, TNFRSF6 and LIPA gene or portion(s) thereof may be obtained in a number of ways. Exemplary methods for obtaining nucleic acid containing a polymorphic uPA, SNCG, IDE, KNSL1, TNFRSF6 and LIPA gene or portion(s) thereof are described herein with respect to methods of generating recombinant cells containing such nucleic acids. For example, a polymorphic uPA, SNCG, IDE, KNSL1, TNFRSF6 and LIPA gene or portion(s) thereof may be obtained by alteration, e.g., site-directed or amplification-mediated mutagenesis, of a wild-type or reference uPA, SNCG, IDE, KNSL1, TNFRSF6 and LIPA gene or cDNA, production of a synthetic nucleic acid using standard techniques known in the art or cloning from a cell source. uPA, SNCG, IDE, KNSL1, TNFRSF6 and LIPA genes or cDNAs may be obtained by employing standard cloning procedures using nucleic acids isolated from cells that express uPA, SNCG, IDE, KNSL1, TNFRSF6 and LIPA protein. Such cells include migratory cells, endothelial cells, chondrocytes and cells of the central nervous system, e.g., neurons and microglia. Human uPA protein is expressed, for example, in human embryonic kidney cells (HEK cells; see, e.g., U.S. Pat. Nos. 4,370,417 and 4,558,010), Hep3 cells (see, e.g., U.S. Pat. No. 5,242,819), the A431 cell line [Fabricant et al. (1977) Proc. Natl. Acad. Sci. U.S.A. 74:565-569 and Stoppelli et al. (1986) Cell 45:675-684], the HT1080 cell line [Andreasen et al. (1986) J. Biol. Chem. 261:7644-7651], the human glioblastoma cell line SNB19 [see, e.g., Mohanam et al. (2001) Clin. Cancer Res. 7:2519-2526] and human glioma cell lines U251, U87 and T98G [see, e.g., Nakada et al. (1999) J. Neuropathol. Exp. Neurol. 58:329-334].

The exogenous nucleic acid containing a polymorphic uPA, SNCG, IDE, KNSL1, TNFRSF6 and LIPA gene or portion(s) thereof that is used in the generation of transgenic animals provided herein contains, in particular embodiments, a sequence of nucleotides that ultimately provides for a product upon transcription of the uPA, SNCG, IDE, KNSL1, TNFRSF6 and LIPA gene or portion(s) thereof. The product can be, for instance, RNA and/or a protein translated from a transcript. For example, the product can be uPA, SNCG, IDE, KNSL1, TNFRSF6 and LIPA mRNA and/or a uPA, SNCG, IDE, KNSL1, TNFRSF6 and LIPA protein or a reporter molecule such as a reporter protein. If the polymorphic uPA, SNCG, IDE, KNSL1, TNFRSF6 and LIPA gene or portion(s) thereof being used in the generation of transgenic animals provided herein does not contain sequences that provide for transcription of the uPA, SNCG, IDE, KNSL1, TNFRSF6 and LIPA gene or portion(s) thereof, any appropriate transcription control sequences, such as a promoter, from any appropriate source which will provide for transcription of the uPA, SNCG, IDE, KNSL1, TNFRSF6 and LIPA gene or portion(s) thereof in the animal can be used. The nucleic acid containing a polymorphic uPA, SNCG, IDE, KNSL1, TNFRSF6 and LIPA gene or portion(s) thereof may be selectively introduced into and/or expressed in particular cell types by utilizing regulatory sequences linked to the uPA, SNCG, IDE, KNSL1, TNFRSF6 and LIPA gene or portion(s) thereof that function in particular cell types. If the polymorphism(s) occur in a transcription control region of a uPA, SNCG, IDE, KNSL1, TNFRSF6 and LIPA gene, the polymorphic control region of the gene can be isolated or synthesized and operatively linked to nucleic acid encoding a reporter molecule,,e.g., β-galactosidase, a fluorescent protein such as green fluorescent protein, or some other readily detectable molecule, or nucleic acid encoding a uPA, SNCG, IDE, KNSL1, TNFRSF6 and LIPA protein. The resultant fusion gene can be used as the transgene that is introduced into an animal cell for use in development of a transgenic animal therefrom. The patterns and levels of expression of the reporter or other molecule in the transgenic animal can be analyzed and compared to those in a transgenic animal containing a fusion gene in which a wild-type or reference uPA, SNCG, IDE, KNSL1, TNFRSF6 and LIPA transcription control region sequence is operatively linked to nucleic acid encoding a reporter or other molecule.

In a particular embodiment, a transgenic animal provided herein contains as a heterologous transgenic element nucleic acid containing one or more uPA gene transcription control regions that include one or more polymorphisms that occur at nucleotide positions corresponding to the following nucleotide positions: 178, 401, 464, 515, 748, 6519, 6532, 6909 and 7235 in SEQ ID NO: 559 or 560 and positions 79, 93, 256, 385 and 714 of SEQ ID NO:563, or the complementary positions thereof. In further embodiments, the one or more uPA gene transcription control regions that include one or more polymorphisms that occur at nucleotide positions corresponding to the following nucleotide positions in SEQ ID NO: 559 or 560: 178, 401, 464, 515, 748, or the complementary positions thereof. In particular embodiments, the nucleotide at position 178 is A or G, at position 401 is G or A, and in particular an A, at position 464 is G or position 464 is deleted, at position 515 is C or T, and in particular a T, and at position 748 is G or T, and in particular a T, or the complements thereof. In yet further embodiments, the one or more uPA gene transcription control regions that include one or more polymorphisms that occur at nucleotide positions corresponding to the following nucleotide positions in SEQ ID NO: 559 or 560: 6519, 6532, 6909 and 7235, or the complementary positions thereof. In particular embodiments, the nucleotide at position 6519 is C or G, at position 6532 is T or C, at position 6909 is G or T, and at position 7235 is G, or the complements thereof, or position 7235 is deleted.

Expression of the exogenous polymorphic uPA, SNCG, IDE, KNSL1, TNFRSF6 and LIPA gene or portion(s) thereof in a transgenic animal can be assessed using standard techniques and compared to the expression of a wild-type or reference uPA, SNCG, IDE, KNSL1, TNFRSF6 and LIPA transgene or portion(s) thereof in a similar transgenic animal. For example, initial screening may be accomplished by Southern blot analysis or nucleic acid amplification techniques to analyze animal tissues to determine whether the exogenous polymorphic uPA, SNCG, IDE, KNSL1, TNFRSF6 and LIPA gene or portion(s) thereof integrated into the genome of the host animal or is present as an extrachromosomal element. The level of mRNA expression from an exogenous polymorphic uPA, SNCG, IDE, KNSL1, TNFRSF6 and LIPA transgene or portion(s) thereof in the tissues of a transgenic animal may be assessed using techniques that include, but are not limited to, Northern blot analysis of tissue samples, in situ hybridization analysis and RT-PCR (reverse transcriptase PCR). uPA, SNCG, IDE, KNSL1, TNFRSF6 and LIPA protein and activity may be detected and/or quantified using various techniques including immunoblot assays, zymography [see, e.g., Vasalli et al. (1984) J. Exp. Med. 159:1653-1668; Sappino et al. (1991) J. Clin. Invest. 88:1073-1079; and Zhou et al. (2000) EMBO J. 19:4817-4826], a plasminogen activation-based assay utilizing fluorogenic fibrin [Wu and Diamond (1995) Thromb. Haemost. 74:711-7171 and a uPA, SNCG, IDE, KNSL1, TNFRSF6 and LIPA activity assay using a two-chain uPA, SNCG, IDE, KNSL1, TNFRSF6 and LIPA-specific fluorogenic substrate: glutamyl-glycyl-arginine-7-amino-4-methyl coumarin [Wolf et al. (1993) J. Biol. Chem. 268:16327-163311. Reporter molecule levels may be determined using assays designed for detection of the particular molecule.

Transgenic animals can comprise other genetic alterations in addition to the presence of alleles of a uPA, SNCG, IDE, KNSL1, TNFRSF6 and LIPA gene or portion(s) thereof. For example, the genome can be altered to affect the function of the endogenous genes, contain marker genes, or contain other genetic alterations (e.g., alleles of other genes associated with disease). Thus, for example, a transgenic animal provided herein containing nucleic acid containing a polymorphic uPA, SNCG, IDE, KNSL1, TNFRSF6 and LIPA gene or portion(s) thereof may be one in which any endogenous uPA, SNCG, IDE, KNSL1, TNFRSF6 and LIPA gene has been deleted or changed such that the function and/or expression of the endogenous gene is altered. The alteration may be one which eliminates or significantly reduces endogenous uPA, SNCG, IDE, KNSL1, TNFRSF6 and LIPA protein and/or activity. The endogenous genome may also be altered to include, for example, a polymorphic gene or portion(s) thereof that is associated with a disease, such as a neurodegenerative disease or disorder. The neurodegenerative disease can be Alzheimer's disease. Thus, in a particular embodiment of the transgenic animals provided herein, the animal contains nucleic acid that contains an APOE4, APP, PS1, PS2 and/or Tau gene or portion thereof. In particular, the nucleic acid contains an APOE4, APP, PS1, PS2 and/or Tau gene or portion thereof that includes one or more polymorphisms that are associated with Alzheimer's disease. For example, transgenic mice that contain an APP transgene element have been developed (see, e.g., Hsiao et al. (1996) Science 274:177-178 (transgenic mice overexpressing the 695-amino acid isoform of human Alzheimer β amyloid (Aβ) precursor protein containing a Lys670→Asn, Met671→Leu mutation) and Hsiao (1998) Exp. Gerontol. 33:883-889).

A “knock-out” of a gene means an alteration in the sequence of the gene that results in a decrease of function of the target gene, preferably such that target gene expression is undetectable or insignificant. “Knock-out” transgenics can be transgenic animals having a heterozygous knock-out of a gene or a homozygous knock-out. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic)) of the target gene, e.g., by introduction of an additional copy of the target gene, or by operatively inserting a regulatory sequence that provides for enhanced expression of an endogenous copy of the target gene. “Knock-in” transgenics of interest can be transgenic animals having a knock-in of an allele associated with neurodegenerative disease including Alzheimer's disease. Such transgenics can be heterozygous or homozygous for the knock-in gene. “Knock-ins” also encompass conditional knock-ins.

Suitable constructs for use in the generation of transgenic animals include, for example, constructs that allow the desired level of expression of a transgene or portion(s) thereof. Methods of isolating and cloning a desired sequence, as well as suitable constructs for expression of a selected sequence in a host animal, are well known in the art and are described herein.

For the generation of transgenic animals, it is generally advantageous to use a nucleic acid construct for introduction of the heterologous nucleic acid into an animal cell wherein the gene; coding sequence or portion(s) thereof is ligated downstream of a promoter capable of and operably linked to the gene, coding sequence or portion(s) thereof for expression of the gene, coding sequence or portion(s) thereof in the subject animal cells. The promoter of the transgene of interest may be used if it will provide for gene expression in the animal.

For example, a transgenic mammal, in particular a non-human mammal, showing high expression of a desired transgene can be created by microinjecting into a fertilized egg of a non-human mammal (e.g., rat fertilized egg) a vector ligated with the gene or portion(s) thereof downstream of various promoters derived from various mammals (e.g., rabbits, dogs, cats, guinea pigs, hamsters, rats, mice etc., preferably rats etc.) capable of expressing a transcription product and/or a corresponding protein.

Vectors include Escherichia coli-derived plasmids, Bacillus subtilis-derived plasmids, yeast-derived plasmids, bacteriophages such as lambda, phage, retroviruses such as Moloney leukemia virus, and animal viruses such as vaccinia virus or baculovirus.

Promoters for gene expression regulation include, for example, promoters for genes derived from viruses (e.g., cytomegalovirus, Moloney leukemia virus, JC virus, breast cancer virus etc.), and promoters for genes derived from various mammals (e.g., humans, rabbits, dogs, cats, guinea pigs, hamsters, rats, mice etc.) and birds (e.g., chickens etc.) (e.g., genes for albumin, insulin II, erythropoietin, endothelin, osteocalcin, muscular creatine kinase, platelet-derived growth factor beta, keratins K1, K10 and K14, collagen types I and II, atrial natriuretic factor, dopamine beta-hydroxylase, endothelial receptor tyrosine kinase (generally abbreviated Tie2), sodium-potassium adenosine triphosphorylase (generally abbreviated Na,K-ATPase), neurofilament light chain, met allothioneins I and IIA, met alloproteinase I tissue inhibitor, MHC class I antigen (generally abbreviated H-2L), smooth muscle alpha actin, polypeptide chain elongation factor 1 alpha (EF-1 alpha), beta actin, alpha and beta myosin heavy chains, myosin light chains 1 and 2, myelin base protein, serum amyloid component, myoglobin, renin etc.).

The above-mentioned vectors can have a sequence for terminating the transcription of the desired messenger RNA in the transgenic animal (generally referred to as terminator); for example, gene expression can be manipulated using a sequence with such function contained in various genes derived from viruses, mammals and birds. The simian virus SV40 terminator etc. is commonly used. Additionally, for the purpose of increasing the expression of the desired gene, various other elements may be included: e.g., the splicing signal and enhancer region of each gene, a portion of the intron of a eukaryotic organism gene may be ligated 5′ upstream of the promoter region, or between the promoter region and the translational region, or 3′ downstream of the translational region as desired.

Nucleic acid containing a transgene, or portion thereof, can be obtained, for example, from genomic DNA of blood, kidney or fibroblast origin from various animals (e.g., humans, rabbits, dogs, cats, guinea pigs, hamsters, rats, mice etc.) or from various commercially available genomic DNA libraries, as a starting material, or using complementary DNA prepared by a known method from RNA ofvblood, kidney or fibroblast origin as a starting material. Also, an exogenous gene can be obtained using complementary DNA prepared by a known method from RNA of human fibroblast origin as a starting material.

The nucleic acid can then be incorporated into a vector to facilitate transfer into an animal cell. If desired, the nucleic acid can be ligated downstream of a promoter (preferably upstream of the translation termination site) as a gene construct capable of being expressed in the transgenic animal.

Nucleic acid constructs for random integration need not include regions of homology to mediate recombination. Where homologous recombination is desired, the constructs will comprise the heterologous transgene element and will include regions of homology to a target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

The transgenic animal can be created by introducing a nucleic acid construct into, for example, an unfertilized egg, a fertilized egg, a spermatozoon or a germinal cell containing a primordial germinal cell thereof, preferably in the embryogenic stage in the development of a non-human mammal (and in particular in the single-cell or fertilized cell stage and generally before the 8-cell phase), by standard means, such as the calcium phosphate method, the electric pulse method, the lipofection method, the agglutination method, the microinjection method, the particle gun method, the DEAE-dextran method and other such method. Also, it is possible to introduce a desired gene into a somatic cell, a living organ, a tissue cell or other cell, by gene transformation methods, and use it for cell culture, tissue culture and any other method of propagation. Furthermore, these cells may be fused with the above-described germinal cell by a commonly known cell fusion method to create a transgenic animal.

For embryonic stem (ES) cells, an ES cell line may be employed, or embryonic cells may be obtained freshly from a host, e.g., mouse, rat, guinea pig, etc. Such cells are grown on an appropriate fibroblast-feeder layer or grown in the presence of appropriate growth factors, such as leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts can be obtained from 4 to 6 week old superovulated females. The ES cells can be trypsinized, and the modified cells injected into the blastocoel of the blastocyst. After injection, the blastocysts are returned to recipient, e.g., pseudopregnant, females. Females are then allowed to go to term and the resulting offspring screened for cells having the construct. By providing for a different phenotype of the blastocyst and the ES cells, chimeric progeny can be readily detected. Chimeric animals may be screened for the presence of the modified gene. Males and females having the modification can be mated to produce homozygous progeny. If the gene alterations cause lethality at some point in development, tissues or organs can be maintained as allogeneic or congenic grafts or transplants, or in vitro culture.

Animals containing more than one transgene can be made by sequentially introducing individual alleles into an animal in order to produce the desired phenotype (e.g., a structural, molecular, or functional event associated with a disease or disorder). For example, a desired phenotype may be that of a neurodegenerative disease, such as AD, and may include amyloid deposition, neuropathological developments, learning and memory deficits and other possible neurodegenerative disease-associated characteristics.

For example, transgenic mouse models for Alzheimer's disease have been proposed which encode human or murine Alzheimer Related Membrane Protein (ARMP) homologues mutated to manifest an Alzheimer phenotype (see U.S. Pat. No. 6,210,919).

Numerous transgenic mice exhibiting various characteristics of AD and other neurodegenerative diseases are available. These have been made using APP, PS1, PS2, Tau; APOE and other genes, alone and in combinations (see www.alzforum.org/members/resources/transgenic/index.html).

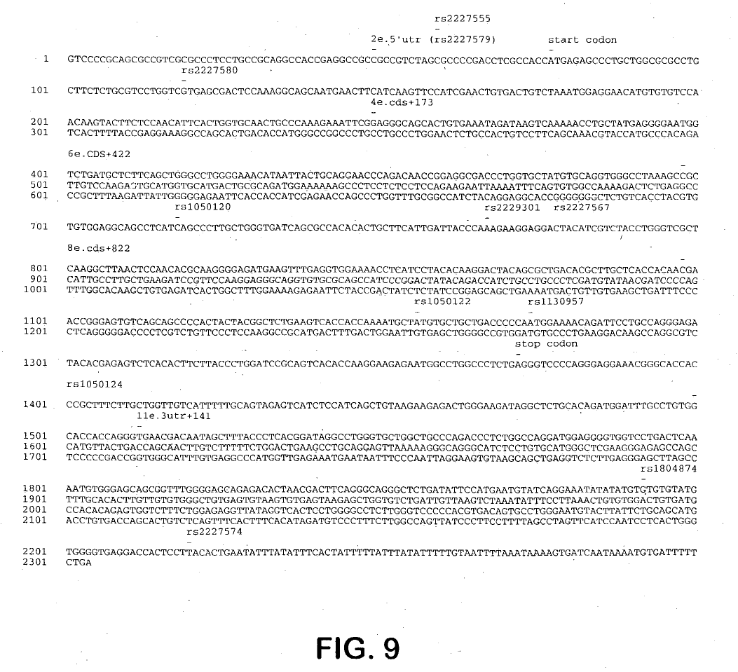

Transgenic animals can be made containing any alleles of a uPA, SNCG, IDE, KNSL1, TNFRSF6 and LIPA gene, either individually or in combinations. Those of ordinary skill would be able to determine appropriate sequences to be utilized based on the sequences of the uPA, SNCG, IDE, KNSL1, TNFRSF6 and LIPA gene and cDNA provided herein. An example of a useful uPA sequence is a sequence with one or more of the polymorphisms indicated in FIG. 9 and SEQ ID NO:563. FIG. 9 and SEQ ID NO:563 indicate the positions in a cDNA sequence that correspond to the following polymorphic regions described in Tables 12 and F for a uPA gene: 2e.5′utr (49), rs2227555 (59), rs2227580 (119), 4e.cds+173 (249), 6e.cds+422 (498), rs1050120 (718), rs2229301 (rs2227567 (767), 8e.cds+822 (898), rs1050122 (1156), rs1130957 (1174), rs1050124 (1500), 11e.utr+141 (1512), rs1804874 (1892) and rs2227574 (2220).

O. Allelic Variants

An allelic variant, depending on its location in the gene, can play various roles in the manifestation of a disease condition. An allelic variant can produce its effect at the level of RNA or protein. Effects on RNA include altered splicing, stability, editing and expression. Effects on the protein include altered protein function, folding, transport, localization, stability and expression. Polymorphisms located in the 5′ untranslated region of the gene may alter the activity of an element of the gene promoter and change the expression of the mRNA (e.g., level or timing of expression). Polymorphisms located in introns may alter RNA stability, editing, splicing, etc. SNPs located in the 3′ untranslated region may influence polyadenylation, or mRNA stability. Silent alteration in the coding region of an gene may affect codon usage or splicing. Changes in amino acids, deletions or insertions may affect protein function by increasing or decreasing a native function or bringing about an altered function. The effect of a polymorphism can be determined by producing transgenic animals in which the allelic variant has been introduced and in which the wild-type gene or predominant allele may have been knocked out. RNA and/or protein is compared in the transgenic mice harboring the allelic variant with transgenic mice harboring the predominant allele. For example, the variant may result in alterations of RNA levels or RNA stability or in increased or decreased synthesis of the associated protein and/or aberrant tissue distribution or intracellular localization of the associated protein, altered phosphorylation, glycosylation and/or altered activity of the protein. Furthermore, various molecular, cellular and organismal manifestations of AD can be monitored; such as APP gene products, neurite plaques, memory and learning and neurodegeneration of specific systems of cells. Such analysis could also be performed in cultured cells, in which the human variant allele gene is introduced and, e.g., replaces the endogenous gene in the cell. These effects can be determined according to methods known in the art and as described below. Allelic variants can be assayed individually or in combination.

1. RNA Analysis

a. Northern Blot Detection of RNA

The northern blot technique is used to identify a RNA fragment of a specific size from a complex population of RNA using gel electrophoresis and nucleic acid hybridization. Northern blotting is a well-known technique in the art. Northern blot analysis is commonly used to detect specific RNA transcripts expressed in a variety of biological samples and have been described in Sambrook, J. et al. (2000) “Molecular Cloning,” 3d ed. Cold Spring Harbor Press.

Briefly, total RNA is isolated from any biological sample by the method of Chomczynski and Sacchi (1987) Anal. Biochem. 162:156-159. Poly-adenylated mRNA is purified from total RNA using mini-oligo (dT) cellulose spin column kit with methods as outlined by the suppliers (Invitrogen, Carlsbad Calif.). Denatured RNA is electrophoresed through a denaturing 1.5% agarose gel and transferred onto a nitrocellulose or nylon based matrix. The mRNAs are detected by hybridization of a radiolabeled or biotinylated oligonucleotide probe specific to the polymorphic regions as disclosed herein.

b. Dot Blot/Slot Blot

Specific RNA transcripts can be detected using dot and slot blot assays to evaluate the presence of a specific nucleic acid sequence in a complex mix of nucleic acids. Specific RNA transcripts can be detected by adding the RNA mixture to a prepared nitrocellulose or nylon membrane. RNA is detected by the hybridization of a radiolabeled or biotinylated oligonucleotide probe complementary to the sequences as disclosed herein.

c. RT-PCR

The RT-PCR reaction may be performed, as described by K.-Q. Hu et al. (1991) Virology 181:721-726, as follows: the extracted mRNA is transcribed in a reaction mixture of 1 micromolar antisense primer, and 25 U AMV (avian myeloblastosis virus) or MMLV (Moloney murine leukemia virus) reverse transcriptase. Reverse transcription is performed and the cDNA is amplified in a PCR reaction volume with Taq polymerase. Optimal conditions for cDNA synthesis and thermal cycling can be readily determined by those skilled in the art.

2. Protein and Polypeptide Detection

a. Expression of Protein in a Cell Line

Using the disclosed nucleic acid sequences and others that can be obtained using methods described herein proteins of interest may be expressed in a recombinantly engineered cell such as a bacterial, yeast, insect, mammalian, or plant cell. Those of ordinary skill in the art are knowledgeable of the numerous expression systems available for expression of a nucleic acid encoding proteins, including polymorphic proteins.

b. Expression of Proteins

The isolated nucleic acid encoding a full-length polymorphic protein, or, a portion thereof, such as a fragment containing the site of the polymorphism, may be introduced into a vector for transfer into host cells. Fragments of the polymorphic proteins can be produced by those skilled in the art, without undue experimentation, by eliminating portions of the coding sequence from the isolated nucleic acids encoding the full-length proteins.

Expression vectors are used when expression of the protein in the host cell is desired. An expression vector includes vectors capable of expressing nucleic acids that are operatively linked with regulatory sequences, such as promoter regions, that are capable of affecting expression of such nucleic acids. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. Such plasmids for expression of polymorphic human uPA, SNCG, IDE, LIPA, TNFRSF6 or KNSL1 encoding nucleic acids in eukaryotic host cells, particularly mammalian cells, include cytomegalovirus (CMV) promoter-containing vectors, such as pCMV5, the pSV2dhfr expression vectors, which contain the SV40 early promoter, mouse dhfr gene, SV40 polyadenylation and splice sites and sequences necessary for maintaining the vector in bacteria, and MMTV promoter-based vectors.

The nucleic acids encoding polymorphic proteins, and vectors and cells containing the nucleic acids as provided herein permit production of the polymorphic proteins, as well as antibodies to the proteins. This provides a means to prepare synthetic or recombinant polymorphic proteins and fragments thereof that are substantially free of contamination from other proteins, the presence of which can interfere with analysis of the polymorphic proteins. In addition, the polymorphic proteins may be expressed in combination with selected other proteins that the protein of interest may associate with in cells. The ability to selectively express the polymorphic proteins alone or in combination with other selected proteins makes it possible to observe the functioning of the recombinant polymorphic proteins within the environment of a cell. The expression of isolated nucleic acids encoding a protein will typically be achieved by operably linking, for example, the DNA or cDNA to a promoter (which is either constitutive or regulatable), followed by incorporation into an expression vector.

The vectors can be suitable for replication and integration in either prokaryotes or eukaryotes. Typical expression vectors contain transcription and translation terminators, initiation sequences, and promoters useful for regulation of the expression of the DNA encoding a protein. To obtain high level expression of a cloned gene, it is desirable to construct expression vectors which contain, a strong promoter to direct transcription, a ribosome binding site for translational initiation, and a transcription/translation terminator. One of ordinary skill in the art would recognize that modifications can be made to a protein without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced. There are expression vectors that specifically allow the expression of functional proteins. One such vector, Plasmid 577, described in U.S. Pat. No. 6,020,122 and incorporated herein by reference, has been constructed for the expression of secreted antigens in a permanent cell line. This plasmid contains the following DNA segments: (a) a fragment of pBR322 containing bacterial beta-lactamase and origin of DNA replication; (b) a cassette directing expression of a neomycin resistance gene under control of HSV-1 thymidine kinase promoter and poly-A addition signals; (c) a cassette directing expression of a dihydrofolate reductase gene under the control of a SV-40 promoter and poly-A addition signals; (d) cassette directing expression of a rabbit immunoglobulin heavy chain signal sequence fused to a modified hepatitis C virus (HCV) E2 protein under the control of the Simian Virus 40 T-Ag promoter and transcription enhancer, the hepatitis B virus surface antigen (HBsAg) enhancer I followed by a fragment of Herpes Simplex Virus-1 (HSV-1) genome providing poly-A addition signals; and (e) a fragment of Simian Virus 40 genome late region of no function in this plasmid. All of the segments of the vector were assembled by standard methods known to those skilled in the art of molecular biology. Plasmids for the expression of uPA, SNCG, IDE, LIPA, TNFRSF6 or KNSL1 proteins can be constructed by replacing the hepatitis C virus E2 protein coding sequence in plasmid 577 with a uPA, SNCG, IDE, LIPA, TNFRSF6 or KNSL1 coding sequence or a fragment thereof (see above). The resulting plasmid is transfected into CHO/dhfr-cells (DXB-111) (Uriacio, et al. (1980) PNAS 77:4451-4466; these cells are available from the A.T.C.C., 12301 Parklawn Drive, Rockville, Md. 20852, under Accession No. CRL 9096), using the cationic liposome-mediated procedure (P. L. Feigner et al. (1987) PNAS 84:7413-7417). Proteins are secreted into the cell culture media.

Incorporation of cloned DNA into a suitable expression vector, transfection of cells with a plasmid vector or a combination of plasmid vectors, each encoding one or more distinct proteins or with linear DNA, and selection of transfected cells are well known in the art (see, e.g., Sambrook et al. (1989) “Molecular Cloning: A Laboratory Manual,” 2d ed. Cold Spring Harbor Laboratory Press). Heterologous nucleic acid may be introduced into host cells by any method known to those of skill in the art, such as transfection with a vector encoding the heterologous nucleic acid by CaPO₄ precipitation (see, e.g., Wigler et al. (1979) Proc. Natl. Acad. Sci. U.S.A. 76:1373-1376) or lipofectamine (Invitrogen, Carlsbad, Calif.). Recombinant cells can then be cultured under conditions whereby the polymorphic human uPA, SNCG, IDE, LIPA, TNFRSF6 or KNSL1 protein encoded by the nucleic acid is expressed. Suitable host cells include mammalian cells (e.g., HEK293, including but are not limited to, those described in U.S. Pat. No. 5,024,939 to Gorman (see, also, Stillman et al. (1985) Mol. Cell. Biol. 5:2051-2060); also, HEK293 cells available from ATCC under accession #CRL 1573), CHO, COS, BHKBI and Ltk⁻ cells, mouse monocyte macrophage P388D1 and J774A-1 cells (available from ATCC, Rockville, Md.) and others known to those of skill in this art), yeast cells, including, but are not limited to, Pichia pastoris, Saccharomyces cerevisiae, Candida tropicalis, Hansenula polymorpha, human cells and bacterial cells, including, but are not limited to, Escherichia coli. Xenopus oöcytes may also be used for expression of in vitro RNA transcripts of the DNA.

Heterologous nucleic acid may be stably incorporated into cells or may be transiently expressed using methods known in the art. Stably transfected mammalian cells may be prepared by transfecting cells with an expression vector having a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a reporter gene (such as the E. coli β-galactosidase gene) to monitor transfection efficiency. Selectable marker genes are not included in the transient transfections because the transfectants are typically not grown under selective conditions, and are usually analyzed within a few days after transfection.

Heterologous nucleic acid may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, the polymorphic human proteins or fragments thereof may be purified using protein purification methods known to those of skill in the art. For example, antibodies or other ligands that specifically bind to the proteins may be used for affinity purification and immunoprecipitation of the proteins.

c. Protein Purification

The proteins may be purified by standard techniques well known to those of skill in the art. Recombinantly produced polymorphic proteins can be directly expressed or expressed as a fusion protein. The recombinant protein is purified by a combination of cell lysis (e.g., sonication, French press) and affinity chromatography. The proteins, recombinant or synthetic, may be purified to substantial purity by standard techniques well known in the art, including detergent solubilization, selective precipitation with such substances as ammonium sulfate, column chromatography, immunopurification methods, and others. (See, for example, R. Scopes (1982) “Protein Purification: Principles and Practice,” Springer-Verlag (New York); Deutscher (1990) “Guide to Protein Purification,” Academic Press). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

Exemplary polymorphic proteins provided herein include an isolated KNSL1 polymorphic protein, comprising an amino acid sequence selected from the group consisting of SEQ ID NO:472, SEQ ID NO:474, and SEQ ID NO:476. In a particular embodiment, the amino acid at position 869 of SEQ ID NOs:472, 474 and 476 is a cysteine.

3. Immunodetection

Generally, the proteins, when presented as an immunogen, should elicit production of a specifically reactive antibody. Immunoassays for determining binding are well known to those of skill in the art, as are methods of making and assaying for antibody binding specificity/affinity. Exemplary immunoassay formats include ELISA, competitive immunoassays, radioimmunoassays, Western blots, indirect immunofluorescent assays, in vivo expression or immunization protocols with purified protein preparations. In general, the detection of immunocomplex formation is well known in the art and may be achieved by methods generally based upon the detection of a label or marker, such as any of the radioactive, fluorescent, biological or enzymatic tags. Labels are well known to those skilled in the art (see U.S. Pat. Nos. 3,817,837; 3,850,752; 3,939,350; 3,996,345; 4,277,437; 4,275,149 and 4,366,241, each incorporated herein by reference). Of course, one may find additional advantages through the use of a secondary binding ligand such as a second antibody or a biotin/avidin ligand binding arrangement, as is known in the art.

a. Production of Polyclonal Antisera

Antibodies can be raised to specific proteins, including individual, allelic, strain, or species variants, and fragments thereof, both in their naturally occurring (full-length) forms and in recombinant forms. Additionally, antibodies are raised to these proteins in either their native configurations or in non-native configurations. Anti-idiotypic antibodies can also be generated. A variety of analytic methods are available to generate a hydrophilicity profile of proteins. Such methods can be used to guide the artisan in the selection of peptides for use in the generation or selection of antibodies which are specifically reactive, under immunogenic conditions. See, e.g., J. Janin (1979) Nature 277:491-492; Wolfenden et al. (1981) Biochemistry 20:849-855; Kyte and Doolite (1982) J. Mol Biol. 157:105-132; Rose et al. (1985) Science 229:834-838.

A number of immunogens can be used to produce antibodies specifically reactive with specific proteins. An isolated recombinant, synthetic, or native polynucleotide are the preferred immunogens (antigen) for the production of monoclonal or polyclonal antibodies. Polypeptides are typically denatured, and optionally reduced, prior to formation of antibodies for screening expression libraries or other assays in which a putative protein is expressed or denatured in a non-native secondary, tertiary, or quaternary structure.

The protein or a portion thereof is injected into an animal capable of producing antibodies. Either monoclonal or polyclonal antibodies can be generated for subsequent use in immunoassays to measure the presence and quantity of the protein. Methods of producing polyclonal antibodies are known to those of skill in the art. In brief, an immunogen (antigen), preferably a purified protein, a protein coupled to an appropriate carrier (e.g., GST, keyhole limpet hemanocyanin, etc.), or a protein incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the protein of interest. When appropriately high titers of antibody to the immunogen are obtained, blood is collected from the animal and antisera are prepared. Further fractionation of the antisera to enrich for antibodies reactive to the protein is performed where desired (See, e.g., Coligan (1991) “Current Protocols in Immunology,” Wiley/Greene (New York); and Harlow and Lane (1989) “Antibodies: A Laboratory Manual,” Cold Spring Harbor Press (New York)).

Exemplary antibodies to polymorphic proteins provided herein include antibodies, either polymorphic or monoclonal, specific against an isolated KNSL1 polymorphic protein, comprising an amino acid sequence selected from the group consisting of SEQ ID NO:472, SEQ ID NO:474, and SEQ ID NO:476.

b. Western Blotting

Biological samples are homogenized in SDS-PAGE sample buffer (50 mM Tris-HCl, pH 6.8, 100 mM dithiothreitol, 2% SDS, 0.1% bromophenol blue, 10% glycerol), heated at 100° C. for 10 min and run on a 14% SDS-PAGE with a 25 mM Tris-HCl, pH 8.3, 250 mM Glycine, 0.1% SDS running buffer. The proteins are electrophoretically transferred to nitrocellulose in a transfer buffer containing 39 mM glycine, 48 mM Tris-HCl, pH 8.3, 0.037% SDS, 20% methanol. The nitrocellulose is dried at room temperature for 60 min and then blocked with a PBS solution containing either bovine serum albumin or 5% nonfat dried milk for 2 hours at 4° C.

The filter is placed in a heat-sealable plastic bag containing a solution of 5% nonfat dried milk in PBS with a 1:100 to 1:2000 dilution of affinity purified anti-uPA, SNCG, IDE, LIPA, TNFRSF6 or KNSL1 peptide antibodies, incubated at 4° C. for 2 hours, followed by three 10 min washes in PBS. An alkaline phosphatase conjugated secondary antibody (i.e., anti-mouse/rabbit IgG), is added at a 1:200 to 1:2000 dilution to the filter in a 150 mM NaCl, 50 mM Tris-HCl, pH 7.5 buffer and incubated for 1 h at room temperature.

The bands are visualized upon the addition and development of a chromogenic substrate such as 5-bromo-4-chloro-3-indolyl phosphate/nitro blue tetrazolium (BCIP/NBT). The filter is incubated in the solution at room temperature until the bands develop to the desired intensity. Molecular mass determination is made based upon the mobility of pre-stained molecular weight standards (Rainbow markers, Amersham, Arlington Heights, IlI.).

c. Microparticle Enzyme Immunoassay (MEIA)

Proteins and peptides are detected using a standard commercialized antigen competition EIA assay or polyclonal antibody sandwich EIA assay on the lMx.RTM Analyzer (Abbott Laboratories, Abbott Park, IlI.). Samples containing the specific protein are incubated in the presence of antibody coated microparticles. The microparticles are washed and secondary polyclonal antibodies conjugated with detectable entities (i.e., alkaline phosphatase) are added and incubated with the microparticles. The microparticles are washed and the bound antibody/antigenlantibody complexes are detected by adding a substrate (i.e. 4-methyl umbelliferyl phosphate) (MUP) that will react with the secondary conjugated antibody to generate a detectable signal.

d. Immunocytochemistry

Intracellular localization of a specific protein can be determined by a variety of in situ hybridization techniques. In one method cells are fixed with 4% paraformaldehyde in 0.1 M phosphate buffered saline (PBS; pH7.4) for 5 min., rinsed in PBS for 2 min., dilapidated and dehydrated in an ethanol series (50, 70 and 95%) (5 min. each and stored in 95% ethanol at 4° C).

The cells are stained with a primary antibody and a mixture of secondary antibodies used for detection. Laser-scanning confocal microscopy is performed to localize the protein.

P. Cells Containing Isolated Nucleic Acids

The disclosed nucleic acids and others that can be obtained using methods described herein may be transferred into a host cell such as a bacterial, yeast, insect, mammalian, or plant cell for recombinant expression therein. Thus, provided herein are recombinant cells containing a uPA, SNCG, IDE, KNSL1, TNFRSF6 or LIPA gene or a portion or portions thereof, such as, for example, a transcriptional control region (including, for example, a promoter and 3′ untranslated (UTR) sequences) and/or a coding sequence of a uPA, SNCG, IDE, KNSL1, TNFRSF6 or LIPA gene. The uPA, SNCG, IDE, KNSL1, TNFRSF6 or LIPA gene or portion(s) thereof contains at least one polymorphic region and is thus referred to as a polymorphic uPA, SNCG, IDE, KNSL1, TNFRSF6 or LIPA gene or portion(s) thereof. A “uPA, SNCG, IDE, KNSL1, TNFRSF6 or LIPA gene or a portion or portions thereof” includes a uPA, SNCG, IDE, KNSL1, TNFRSF6 or LIPA cDNA or portion(s) thereof. In particular embodiments, the polymorphic uPA, SNCG, IDE, KNSL1, TNFRSF6 or LIPA gene is a human polymorphic uPA, SNCG, IDE, KNSL1, TNFRSF6 or LIPA gene.

Cells containing nucleic acids encoding polymorphic uPA, SNCG, IDE, KNSL1, TNFRSF6 or LIPA proteins, and vectors and cells containing the nucleic acids as provided herein permit production of the polymorphic proteins, as well as antibodies to the proteins. This provides a means to prepare synthetic or recombinant polymorphic proteins and fragments thereof that are substantially free of contamination from other proteins, the presence of which can interfere with analysis of the polymorphic proteins. In addition, the polymorphic proteins may be expressed in combination with selected other proteins that the protein of interest may associate with in cells. The ability to selectively express the polymorphic proteins alone or in combination with other selected proteins makes it possible to observe the functioning of the recombinant polymorphic proteins within the environment of a cell.

Recombinant cells provided herein may be used for numerous purposes. For example, the cells may be used in testing polymorphic uPA, SNCG, IDE, KNSL1, TNFRSF6 or LIPA genes or portion(s) thereof for characterization of phenotypic outcomes correlated with the particular polymorphisms. The cells may also be used in the production of recombinant uPA, SNCG, IDE, KNSL1, TNFRSF6 or LIPA protein. Such protein may be used, for example, in assays for molecules that bind to, and in particular affect the activity of, uPA, SNCG, IDE, KNSL1, TNFRSF6 or LIPA. The proteins may also be used in the production of antibodies specific for the protein. Additionally, the recombinant uPA, SNCG, IDE, KNSL1, TNFRSF6 or LIPA protein may be used as a source of serine protease activity. For example, the recombinant uPA, SNCG, IDE, KNSL1, TNFRSF6 or LIPA may be used to activate plasminogen and generate plasmin used to degrade fibrin. Recombinant cells containing polymorphic uPA, SNCG, IDE, KNSL1, TNFRSF6 or LIPA genes or portion(s) thereof may also be used in methods of identifying agents that modulate uPA, SNCG, IDE, KNSL1, TNFRSF6 or LIPA gene and protein expression and/or activity or that modulate a biological event characteristic of a disease or disorder involving altered uPA, SNCG, IDE, KNSL1, TNFRSF6 or LIPA gene and/or protein expression or function which may be candidate treatments for a disease or disorder.

Also provided herein are methods of producing recombinant cells by introducing a polymorphic uPA, SNCG, IDE, KNSL1, TNFRSF6 or LIPA gene or portion(s) thereof into a cell. The cell may be any transfectable cell. Such cells, and methods of introducing heterologous nucleic acids into the cells, are known to those of skill in the art.

Nucleic acids for use in generating recombinant cells provided herein are nucleic acids containing one or more polymorphic regions of a uPA, SNCG, IDE, KNSL1, TNFRSF6 or LIPA gene and particular polymorphisms thereof, such as particular uPA, SNCG, IDE, KNSL1, TNFRSF6 or LIPA gene alleles. In particular embodiments, the nucleic acid used in generating a recombinant cell contains a human uPA, SNCG, IDE, KNSL1, TNFRSF6 or LIPA gene or portion(s) thereof containing at least one polymorphic region. Of particular interest are variants of a uPA, SNCG,. IDE, KNSL1, TNFRSF6 or LIPA gene or portion(s) thereof that are associated with a disease or disorder, such as a uPA, SNCG, IDE, KNSL1, TNFRSF6 or LIPA-mediated disease or disorder. In particular embodiments of the recombinant cells provided herein, the cell contains a polymorphic uPA, SNCG, IDE, KNSL1, TNFRSF6 or LIPA gene or portion(s) thereof associated with thrombosis, thrombolytic diseases, stroke, atherosclerosis, coronary artery disease, cardiovascular disease, cardiac disorders, myocardial infarction, cardiomyopathies, proliferative diseases, cancer, tumor angiogenesis, tumor metastasis, arthritis, rheumatic diseases and inflammatory diseases, including inflammatory joint disease. In further embodiments of the recombinant cells provided herein, the cell contains a polymorphic uPA, SNCG, IDE, KNSL1, TNFRSF6 or LIPA gene or portion(s) thereof associated With a neurodegenerative disease or disorder. In yet further embodiments of the recombinant cells provided herein, the cell contains a polymorphic uPA, SNCG, IDE, KNSL1, TNFRSF6 or LIPA gene or portion(s) thereof associated with Alzheimer's disease.

Exemplary polymorphic regions and particular allelic variants for use in the recombinant cells provided herein include those set forth in the Examples at Tables 2, 4 and 4-B, 6 and 6-B, 8, 10, 12 and 12-B, and A-F. In a particular embodiment, a recombinant cell provided herein contains heterologous nucleic acid comprising a uPA, SNCG, IDE, KNSL1, TNFRSF6 or LIPA nucleic acid molecule described herein in the “Nucleic Acid Molecules”, “Primers, Probes and Antisense Nucleic Acids” and/or the “cDNAs” sections set forth herein. In another embodiment, a recombinant cell provided herein contains heterologous nucleic acid containing a uPA, SNCG, IDE, KNSL1, TNFRSF6 or LIPA gene or portion(s) thereof that includes one or more polymorphisms associated individually and/or in combination with a uPA, SNCG, IDE, KNSL1, TNFRSF6 or LIPA-mediated disease or disorder. In a further embodiment, a recombinant cell provided herein contains heterologous nucleic acid containing a uPA, SNCG, IDE, KNSL1, TNFRSF6,or LIPA gene or portion(s) thereof that includes one or more polymorphisms associated individually and/or in combination with a neurodegenerative disease or disorder. In particular embodiments, the neurodegenerative disease is Alzheimer's disease. In a yet further embodiment, the neurodegenerative disease is Alzheimer's disease with an age of onset that is greater than or equal to about 50 years, or greater than or equal to about 60 years, or greater than or equal to about 65 years.

In an exemplary particular embodiment, a recombinant cell provided herein contains heterologous nucleic acid containing a uPA gene or portion(s) thereof that includes one or more polymorphisms that occur at nucleotide positions corresponding to the following nucleotide positions in SEQ ID NO: 559 or 560: nucleotide 401 which is an A, T or C; nucleotide 515 which is a T, G or A; nucleotide 748 which is a T, C or A; and nucleotide 1752 which is a C, G or A, or the complementary positions thereof. In a further particular embodiment, the heterologous nucleic acid contains a uPA gene or portion(s) thereof that includes one or more polymorphisms that occur at nucleotide positions corresponding to the following nucleotide positions in SEQ ID NO: 559 or 560, or the complementary positions thereof: nucleotide 401 which is an A; nucleotide 515 which is a T; nucleotide 748 which is a T; and nucleotide 1752 which is a C.

In another embodiment, a recombinant cell provided herein contains heterologous nucleic acid containing a uPA gene or portion(s) thereof that includes one or more polymorphisms associated individually and/or in combination with a uPA-mediated disease or disorder and that occur at nucleotide positions corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 of SEQ ID NO:563, or the complementary positions thereof.

In a particular embodiment, the recombinant cell contains heterologous nucleic acid containing a uPA gene or portion(s) thereof that includes one or more polymorphisms associated with a uPA-mediated disease or disorder in combination with one or more polymorphisms that occur at a nucleotide position corresponding to the following nucleotide positions in SEQ ID NO: 559 or 560, or the complementary positions thereof: nucleotide 401 which is an A, T or C, and in particular an A; nucleotide 515 which is a T, G or A, and in particular a T; nucleotide 748 which is a T, C or A, and in particular a T; and nucleotide 1752 which is a C, G or A, and in particular a C. In a further embodiment, the one or more polymorphisms associated with a uPA-mediated disease or disorder in combination with one or more of these polymorphisms at nucleotides corresponding to positions 401, 515, 748 and 1752 of SEQ ID NO: 559 or 560 occur at nucleotide positions corresponding to the following nucleotide positions: nucleotide 464, 1229, 1356, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 of SEQ ID NO:563, or the complementary positions thereof.

In another embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 of SEQ ID NO:563, or the complementary positions thereof. In a particular embodiment, the neurodegenerative disease is Alzheimer's disease. In a particular embodiment, the disease is Alzheimer's disease with an age of onset that is greater than or equal to about 50 years, or greater than or equal to about 60 years, or greater than or equal to about 65 years.

In a further embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 9, 401, 464, 515, 748, 1229, 1356, 1752, 1942, 2127, 2543, 3029, 3169, 3799, 3947, 4808, 5287, and 6532, or the complementary positions thereof. In a yet further embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 9, 401, 464, 515, 748, 1229, 1356, 1752, 1942, 2127, 2543, 3029 and 5287, or the complementary positions thereof. In another embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 3169, 3947 and 6532, or the complementary positions thereof. In a particular embodiment, the nucleotide at position 3169 is T, at position 3947 is C, and at position 6532 is T. In another embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions: 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotide positions 79, 93, 256, 385 and 714 of SEQ ID NO:563, or the complementary positions thereof. In yet another embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions: 178, 401, 464, 515 and 748 in SEQ ID NO: 559 or 560 and positions 79, 93, 256, 385 and 714 of SEQ ID NO:563, or the complementary positions thereof. In a further embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 401, 515 and 748, or the complementary positions thereof. In a further embodiment, a recombinant cell provided herein contains heterologous nucleic acid containing a uPA gene or portion(s) thereof that includes one or more polymorphisms associated individually and/or in combination with a neurodegenerative disease or disorder and that occur at nucleotide positions corresponding to the following nucleotide positions of SEQ ID NO: 559 or 560: 6519, 6532, 6909 and 7235, or the complementary positions thereof. In particular embodiments, the neurodegenerative disease is Alzheimer's disease. In a yet further embodiment, the neurodegenerative disease is Alzheimer's disease with an age of onset that is greater than or equal to about 50 years, or greater than or equal to about 60 years, or greater than or equal to about 65 years.

In particular embodiments of any of the above embodiments of the recombinant cells provided herein, the nucleotide at position 9 is A or C, at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, at position 6532 is T or C, at position 178 is A or G, at position 1363 is C or A, at position 1423 is G or T, at position 1465 is C or A, at position 1540 is C or T, at position 2297 is C or T, at position 2445 is T or G, at position 2653 is G or A, at position 3080 is G or A, at position 3546 is C or G, at position 3664 is C or T, at position 3816 is A or C, at position 4320 is T or C, at position 4369 is G or A, at position 4399 is C or A, at position 4851 is G or A, at position 5186 is G or A, at position 5204 is G or A, at position 5787 is C or G, at position 6519 is C or G, at position 6909 is G or T, at position 7235 is G or position 7235 is deleted, at position 7848 is C or T, at position 7908 is A or C; and the nucleotide in SEQ ID NO:563: at position 79 is T or C, at position 93 is a C or position 93 is deleted, at position 256 is G or T, at position 385 is C or T, at position 714-715 is the dinulceotide -GT- or the -GT- dinucleotide is deleted, or the complements thereof.

An isolated nucleic acid containing a uPA, SNCG, IDE, KNSL1, TNFRSF6 or LIPA gene variant sequence, or a portion or portions thereof, that includes the site(s) of one or more polymorphisms, may be introduced into a vector for transfer into host cells. A polymorphic uPA, SNCG, IDE, KNSL1, TNFRSF6 or LIPA gene or portion(s) thereof may be obtained in a number of ways. For example, a polymorphic uPA, SNCG, IDE, KNSL1, TNFRSF6 or LIPA gene or portion(s) thereof may be obtained by alteration, e.g., site-directed or amplification-mediated mutagenesis, of a wild-type or reference uPA, SNCG, IDE, KNSL1, TNFRSF6 or-LIPA gene or cDNA, production of a synthetic nucleic acid using standard techniques known in the art or by genomic or cDNA cloning from a cell source. uPA, SNCG, IDE, KNSL1, TNFRSF6 or LIPA genes or cDNAs may be obtained by employing standard cloning procedures using nucleic acids isolated from cells that express uPA, SNCG, IDE, KNSL1, TNFRSF6 or LIPA. protein (uPA, SNCG, IDE, KNSL1, TNFRSF6 or LIPA gene genomic clones may be obtained from any source of genomic DNA). Such cells include migratory cells, endothelial cells, chondrocytes and cells of the central nervous system, e.g., neurons and microglia. Human uPA protein is expressed, for example, in human embryonic kidney cells (HEK cells; see, e.g., U.S. Pat. Nos. 4,370,417 and 4,558,010), Hep3 cells (see, e.g., U.S. Pat. No. 5,242,819), the A431 cell line [Fabricant et al. (1977) Proc. Natl. Acad. Sci. U.S.A. 74:565-569 and Stoppelli et al. (1986) Cell 45:675-684], the HT1080 cell line [Andreasen et al. (1986) J. Biol. Chem. 261:7644-7651], the human glioblastoma cell line SNB19 [see, e.g., Mohanam et al. (2001) Clin. Cancer Res. 7:2519-25261 and human glioma cell lines U251, U87 and T98G [see, e.g., Nakada et al. (1999) J. Neuropathol. Exp. Neurol. 58:329-3341.

The exogenous nucleic acid containing a polymorphic uPA, SNCG, IDE, KNSL1, TNFRSF6 or LIPA gene or portion(s) thereof that is used in the generation of recombinant cells provided herein contains, in particular embodiments, a sequence of nucleotides that ultimately provides for a product upon transcription of the uPA, SNCG, IDE, KNSL1, TNFRSF6 or LIPA gene or portion(s) thereof. The product can be, for instance, RNA and/or a protein translated from a transcript. For example, the product can be uPA, SNCG, IDE, KNSL1, TNFRSF6 or LIPA mRNA and/or a uPA, SNCG, IDE, KNSL1, TNFRSF6 or LIPA protein or a reporter molecule such as a reporter protein. If the polymorphic uPA, SNCG, IDE, KNSL1, TNFRSF6 or LIPA gene or portion(s) thereof being used in the generation of recombinant cells provided herein does not contain sequences that provide for transcription of the uPA, SNCG, IDE, KNSL1, TNFRSF6 or LIPA gene or portion(s) thereof, any appropriate transcription control sequences, such as a promoter, from any appropriate source which will provide for transcription of the uPA, SNCG, IDE, KNSL1, TNFRSF6 or LIPA gene or portion(s) thereof in the cell can be used. If the polymorphism(s) occur in a transcription control region of a uPA, SNCG, IDE, KNSL1, TNFRSF6 or LIPA gene, the polymorphic control region of the gene can be isolated or synthesized and operatively linked to nucleic acid encoding a reporter molecule, e.g., β-galactosidase, a fluorescent protein such as green fluorescent protein, or some other readily detectable molecule, or nucleic acid encoding a uPA, SNCG, IDE, KNSL1, TNFRSF6 or LIPA protein. The resultant fusion gene can be used as the transgene that is introduced into a host cell for use in development of recombinant cells therefrom. The patterns and levels of expression of the reporter or other molecule in the recombinant cells can be analyzed and compared to those in cells containing a fusion gene in which a wild-type or reference uPA, SNCG, IDE, KNSL1, TNFRSF6 or LIPA transcription control region sequence is operatively linked to nucleic acid encoding a reporter or other molecule.

In a particular embodiment, a recombinant cell provided herein contains heterologous nucleic acid containing one or more uPA gene transcription control regions that include one or more polymorphisms that occur at nucleotide positions corresponding to the following nucleotide positions: 178, 401, 464, 515, 748, 6519, 6532, 6909 and 7235 in SEQ ID NO: 559 or 560 and positions 79, 93, 256, 385 and 714 of SEQ ID NO:563, or the complementary positions thereof. In further embodiments, the one or more uPA gene transcription control regions that include one or more polymorphisms occur at nucleotide positions corresponding to the following nucleotide positions in SEQ ID NO: 559 or 560: 178, 401, 464, 515, 748, or the complementary positions thereof. In particular embodiments, the nucleotide at position 178 is A or G, at position 401 is G or A, and in particular an A, at position 464 is G or position 464 is deleted, at position 515 is C or T, and in particular a T, and at position 748 is G or T, and in particular a T, or the complements thereof. In yet further embodiments, the one or more uPA gene transcription control regions that include one or more polymorphisms occur at nucleotide positions corresponding to the following nucleotide positions in SEQ ID NO: 559 or 560: 6519, 6532, 6909 and 7235, or the complementary positions thereof. In particular embodiments, the nucleotide at position 6519 is C or G, at position 6532 is T or C, at position 6909 is G or T, and at position 7235 is G, or the complementary positions thereof, or position 7235 is deleted.

The expression of isolated nucleic acids encoding a protein is typically achieved by incorporating a nucleic acid, e.g., DNA or cDNA, encoding the protein in operative linkage with a promoter (which can be constitutive and/or regulatable) into an expression vector. Expression vectors are used when expression of the protein in the host cell is desired. An expression vector includes vectors capable of expressing nucleic acids that are operatively linked with regulatory sequences, such as promoter regions, that are capable of affecting expression of such nucleic acids. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. Such plasmids for expression of polymorphic uPA, SNCG, IDE, KNSL1, TNFRSF6 or LIPA-encoding nucleic acids in eukaryotic host cells, particularly mammalian cells, include cytomegalovirus (CMV) promoter-containing vectors, such as pCMV5, the pSV2dhfr expression vectors, which contain the SV40 early promoter, mouse dhfr gene, SV40 polyadenylation and splice sites and sequences necessary for maintaining the vector in bacteria, and MMTV promoter-based vectors.

The vectors can be suitable for replication and integration in either prokaryotes or eukaryotes. Typical expression vectors contain transcription and translation terminators, initiation sequences, and promoters useful for regulation of the expression of the DNA encoding a protein. To obtain high level expression of a cloned gene, it is desirable to construct expression vectors which contain a strong promoter to direct transcription, a ribosome binding site for translational initiation, and a transcription/translation terminator. One of ordinary skill in the art would recognize that modifications can be made to a protein without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced. There are expression vectors that specifically allow the expression of functional proteins. One such vector, Plasmid 577, described in U.S. Pat. No. 6,020,122 and incorporated herein by reference, has been constructed for the expression of secreted antigens in a permanent cell line. This plasmid contains the following DNA segments: (a) a fragment of pBR322 containing bacterial beta-lactamase and origin of DNA replication; (b) a cassette directing expression of a neomycin resistance gene under control of HSV-1 thymidine kinase promoter and poly-A addition signals; (c) a cassette directing expression of a dihydrofolate reductase gene under the control of a SV-40 promoter and poly-A addition signals; (d) cassette directing expression of a rabbit immunoglobulin heavy chain signal sequence fused to a modified hepatitis C virus (HCV) E2 protein under the control of the Simian Virus 40 T-Ag promoter and transcription enhancer, the hepatitis B virus surface antigen (HBsAg) enhancer I followed by a fragment of Herpes Simplex Virus-1 (HSV-1) genome providing poly-A addition signals; and (e) a fragment of Simian Virus 40 genome late region of no function in this plasmid. All of the segments of the vector were assembled by standard methods known to those skilled in the art of molecular biology. Plasmids for the expression of uPA, SNCG, IDE, KNSL1, TNFRSF6 or LIPA proteins can be constructed by replacing the hepatitis C virus E2 protein coding sequence in plasmid 577 with a uPA, SNCG, IDE, KNSL1, TNFRSF6 or LIPA coding sequence or a fragment thereof (see above). The resulting plasmid can be transfected into CHO/dhfr-cells (DXB-111) (Uriacio, et al. (1980) PNAS 77:4451-4466; these cells are available from the A.T.C.C., 12301 Parklawn Drive, Rockville, Md. 20852, under Accession No. CRL 9096), using, for example, the cationic liposome-mediated procedure (P. L. Felgner et al. (1987) PNAS 84:7413-7417). Proteins can be secreted into the cell culture media.

Incorporation of cloned nucleic acids into a suitable vector, transfection of cells with a plasmid vector or a combination of plasmid vectors, each encoding one or more distinct proteins or with linear DNA, and selection of transfected cells are well known in the art (see, e.g., Sambrook et al. (1989) “Molecular Cloning: A Laboratory Manual,” 2d ed. Cold Spring Harbor Laboratory Press). Heterologous nucleic acid may be introduced into host cells by any method known to those of skill in the art, such as transfection with a vector containing the heterologous nucleic acid by CaPO₄ precipitation (see, e.g., Wigler et al. (1979) Proc. Natl. Acad. Sci. U.S.A. 76:1373-1376) or lipofectamine (Invitrogen, Carlsbad, Calif.). Recombinant cells can then be cultured under conditions whereby the polymorphic uPA, SNCG, IDE, KNSL1, TNFRSF6 or LIPA gene or portion(s) thereof is expressed. Suitable host cells include mammalian cells [e.g., HEK293, including but are not limited to, those described in U.S. Pat. No. 5,024,939 to Gorman (see, also, Stillman et al. (1985) Mol. Cell. Biol. 5:2051-2060); also, HEK293 cells available from ATCC under accession #CRL 15731, CHO, COS, BHKBI and Ltk⁻ cells, mouse monocyte macrophage P388D1 and J774A-1 cells (available from ATCC, Rockville, Md.) and others known to those of skill in this art, yeast cells, including, but not limited to, Pichia pastoris, Saccharomyces cerevisiae, Candida tropicalis, Hansenula polymorpha, human cells and bacterial cells, including, but not limited to, Escherichia coli. Xenopus oöcytes may also be used for expression of in vitro RNA transcripts of the DNA.

Heterologous nucleic acid may be stably incorporated into cells or may be transiently expressed using methods known in the art. Stably transfected mammalian cells may be prepared by transfecting cells with an expression vector having a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells may be transfected with a reporter gene (such as the E. coli β-galactosidase gene) to monitor transfection efficiency. Selectable marker genes may not be included in the transient transfections because the transfectants are typically not grown under selective conditions, and are usually analyzed within a few days after transfection.

Heterologous nucleic acid may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, polymorphic proteins or fragments thereof may be purified using protein purification methods known to those of skill in the art. For example, antibodies or other ligands that specifically bind to the proteins may be used for affinity purification and immunoprecipitation of the proteins.

Expression of the exogenous polymorphic uPA, SNCG, IDE, KNSL1, TNFRSF6 or LIPA gene or portion(s) thereof in a recombinant cell can be assessed using standard techniques known to those of skill in the art and described herein and compared to the expression of a wild-type or reference uPA, SNCG, IDE, KNSL1, TNFRSF6 or LIPA transgene or portion(s) thereof in a similar cell. For example, initial screening may be accomplished by Southern blot analysis or nucleic acid amplification techniques to analyze transfected cells to determine whether the exogenous polymorphic uPA, SNCG, IDE, KNSL1, TNFRSF6 or LIPA gene or portion(s) thereof integrated into the genome of the host cell or is present as an extrachromosomal element. The level of mRNA expression from an exogenous polymorphic uPA, SNCG, IDE, KNSL1, TNFRSF6 or LIPA transgene or portion(s) thereof in the recombinant cells may be assessed using techniques that include, but are not limited to, Northern blot analysis of tissue samples, in situ hybridization analysis and RT-PCR (reverse transcriptase PCR). uPA, SNCG, IDE, KNSL1, TNFRSF6 or LIPA protein and activity may be detected and/or quantified using various techniques including immunoblot assays, zymography [see, e.g., Vasalli et al. (1984) J. Exp. Med. 159:1653-1668; Sappino et al. (1991) J. Clin. invest. 88:1073-1079; and Zhou et al. (2000) EMBO J. 19:4817-4826], a plasminogen activation-based assay utilizing fluorogenic fibrin [Wu and Diamond (1995) Thromb. Haemost. 74:711-717] and a uPA, SNCG, IDE, KNSL1, TNFRSF6 or LIPA activity assay using a two-chain uPA, SNCG, IDE, KNSL1, TNFRSF6 or LIPA-specific fluorogenic substrate: glutamyl-glycyl-arginine-7-amino-4-methyl coumarin [Wolf et al. (1993) J. Biol. Chem. 268:16327-163311. Reporter molecule levels may be determined using assays designed for detection of the particular molecule.

Recombinant cells provided herein can comprise other genetic alterations or heterologous nucleic acids in addition to the presence of alleles of a uPA, SNCG, IDE, KNSL1, TNFRSF6 or LIPA gene or portion(s) thereof. For example, the genome can be altered to affect the function of the endogenous genes, contain marker genes, or contain other genetic alterations (e.g., alleles of other genes associated with disease). Thus, for example, a recombinant cell provided herein containing nucleic acid containing a polymorphic uPA, SNCG, IDE, KNSL1, TNFRSF6 or LIPA gene or portion(s) thereof may be one in which any endogenous uPA, SNCG, IDE, KNSL1, TNFRSF6 or LIPA gene has been deleted or changed such that the function and/or expression of the endogenous gene is altered. The alteration may be one which eliminates or significantly reduces endogenous uPA, SNCG, IDE, KNSL1, TNFRSF6 or LIPA protein and/or activity. The endogenous genome may also be altered to include, for example, a polymorphic gene or portion(s) thereof that is associated with a disease, such as a neurodegenerative disease or disorder. The neurodegenerative disease can be Alzheimer's disease. Thus, in a particular embodiment of the recombinant cells provided herein, the cell contains nucleic acid that contains an APOE4, APP, PS1, PS2 and/or Tau gene or portion(s) thereof. In particular, the nucleic acid contains an APOE4, APP, PS1, PS2 and/or Tau gene or portion(s) thereof that includes one or more polymorphisms that are associated with Alzheimer's disease.

Recombinant cells can be made containing any alleles of the uPA, SNCG, IDE, KNSL1, TNFRSF6 or LIPA gene, either individually or in combinations. Those of ordinary skill would be able to determine appropriate sequences to be utilized based on the sequences of the uPA, SNCG, IDE, KNSL1, TNFRSF6 or LIPA gene and cDNA provided herein. An example of a useful sequence is a sequence with one or more of the polymorphisms indicated in FIG. 9 and SEQ ID NO:561. FIG. 9 and SEQ ID NO:561 indicate the positions in a cDNA sequence that correspond to the following polyrnorphic regions described in Tables 12 and F for a uPA gene: 2e.5′utr (49), rs2227555 (59), rs2227580 (119), 4e.cds+173 (249), 6e.cds+422 (498), rs1050120 (718), rs2229301 (rs2227567 (767), 8e.cds+822 (898), rsl1050122 (1156), rsl1130957 (1174), rs1050124 (1500), 11e.utr+141 (1512), rs1804874 (1892) and rs2227574 (2220).

Q. Diagnostic and Prognostic Assays

Typically, an individual allelic variant of the uPA, SNCG, IDE, LIPA, TNFRSF6 and KNSL1 gene will not be used in isolation as an indicator or prognosticator of the disease or protection thereof, unless that allele represents a mutation in a disease gene which is involved in causing neurodegenerative disease, including Alzheimer's disease or a gene conferring protection from a neurodegenerative disease, including Alzheimer's disease. Polymorphisms that are not directly involved in the etiology of the disease may be in linkage-disequilibrium with a yet unidentified disease-causing or disease-protecting polymorphic locus and thus are useful for purposes of diagnostic and prognostic assays to indicate either a predisposition for, or protective effect from (“protection”), the disease.

As used herein, the term “protective” or “protection” with reference to an allele refers to an allele that is indicative of a decreased risk relative to the general population for a genetic disease, e.g., AD. The decreased risk associated with a protective allele may be identified as under-representation of the allele in cases relative to controls, and/or as a significant association between the allele and unaffected members of a family that contains affected members. A protective allele may be a variant of a DNA segment, such as a gene, that has a risk factor or disease allele.

An individual polymorphism or an allelic pattern or haplotype can be used to asses an individual's level of risk for disease, such as a neurodegenerative disease, e.g., AD. In a particular method, individual or haplotype combinations of polymorphisms will be used to indicate whether a subject is predisposed to the development or has a neurodegenerative disease, e.g., AD. Individual polymorphisms and haplotypes useful for diagnostic and prognostic assays are provided herein and can be determined as described herein. In addition, the presence of an individual polymorphism or a haplotype may only be one of a plurality of indicators that are used. The other indicators may be allelic variants or haplotypes in associated genes on other chromosomes, e.g., APOE4, and the manifestation of other risk factors of neurodegenerative disease and other evidence of neurodegenerative disease.

A subject is genotyped for one or more polymorphisms. Polymorphisms can be assayed individually or assayed simultaneously using multiplexing methods as described above or any other labelling method that allows different variants to be identified. In particular, variants of these genes may be assayed using kits (see below) or any of a variety microarrays known to those in the art. For example, oligonucleotide probes comprising the polymorphic regions surrounding any polymorphism in these genes may be designed and fabricated using methods such as those described in U.S. Pat. Nos. 5,492,806; 5,525,464; 5,695,940; 6,018,041; 6,025,136; WO 98/30883; WO 98/56954; WO99/09218; WO 00/58516; WO 00/58519, or references cited therein. A subject's genotype may reflect the presence of the relevant individual polymorphism or haplotype. However, if this is not the case, haplotype information can be derived from the subject's genotype by utilizing an appropriate algorithm such as TRANSMIT (see sections on association and haplotype analysis at pages 60-70). Comparison of the subject's genotype or haplotype with a predetermined reference genotype or haplotype exhibiting association with neurodegenerative disease, e.g., AD, will indicate whether the subject has a predisposition or the occurrence of neurodegerative disease. Haplotyping or genotyping can also be carried out, similarly, for association with protection.

An exemplary haplotype useful in the methods provided herein for assessing level of risk for neurodegenerative disease (e.g., AD) or determining a predisposition or occurrence of neurodegenerative disease, such as Alzheimer's disease, comprises multiple polymorphic regions of the IDE gene corresponding to nucleotides 2456, 3279, 3407 and 42943 of SEQ ID NO:187. In one embodiment, the nucleotide in IDE at position 2456 of SEQ ID NO:187 is G, at position 3279 of SEQ ID NO:187 is T, at position 3407 of SEQ ID NO:187 is T, and at position 42943 of SEQ ID NO:187 is T. In another embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO::187 is T. In still a further embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is C. In yet another embodiment, the nucleotide in IDE at position 2456 of SEQ ID NO:187 is T, at position 3279 of SEQ ID NO:187 is C, at position 3407 of SEQ ID NO:187 is C, and at position 42943 of SEQ ID NO:187 is C. Also for use in the methods provided herein are IDE gene haplotypes that are subsets of this haplotype which have fewer (i.e., three) polymorphisms in the haplotype. In one such haplotype of the IDE gene that associates with risk for AD, the SNPs correspond to nucleotide positions 2456, 3279 and 42943 of SEQ ID NO:186 (positions 121239, 120416 and 80752 of SEQ ID NO:484) and, in a particular embodiment, the nucleotide at each position with respect to the sequence shown in SEQ ID NO: 186 is a G, T, T (or the complements thereof in the complementary positions). In another such haplotype of the IDE gene that associates with risk for AD, the SNPs correspond to nucleotide positions 3279, 3407 and 42943 of SEQ ID NO:186 (positions 120416, 120288 and 80752 of SEQ ID NO:484) and, in a particular embodiment, the nucleotides at the positions with respect to the sequence shown in SEQ ID NO:186 are T, C and T (or the complements thereof in the complementary positions).

Another IDE gene haplotype useful in the methods provided herein for assessing level of risk for neurodegenerative disease (e.g., AD) includes 5 SNPs, combinations of which associate with risk for or protection against AD, corresponding to nucleotide positions 122260, 120416, 120288, 80752 and 54795 of SEQ ID NO:484, or the complementary positions thereof. In a particular embodiment, the nucleotides at the positions are G, A, G, A and G, respectively, or the complements thereof in the complementary positions, and the haplotype is associated with risk for disease. In another embodiment, the nucleotides at the positions are A, A, G, A and G, respectively, or the complements thereof in the complementary positions, and the haplotype is associated with protection against AD.

An exemplary haplotype useful in the methods provided herein for assessing level of risk for neurodegenerative disease (e.g., AD) or determining a predisposition or occurrence of neurodegenerative disease, such as Alzheimer's disease, comprises multiple polymorphic regions of the KNSL1 gene corresponding to nucleotides 132370, 133355, 147842 and 178981 of SEQ ID NO:484. In one embodiment, the nucleotide(s) in KNSL1: at position 132370 of SEQ ID NO:484 is A; between positions 133354-133355 of SEQ ID NO:484 is a 6, 7 or 8 base pair poly-T insertion corresponding to -TTTTTT(T)(T)-; at positions 147842-147845 of SEQ ID NO:484 is the 4 base pair insertion corresponding to -AGTT-; and between positions 178980-178981 of SEQ ID NO:484 is the 5 base pair insertion corresponding to -AATTT-. In particular embodiments, the poly-T insertion can be 6 base pairs corresponding to -TTTTTT-; the poly-T insertion can be 7 base pairs corresponding to -TTTTTTT-; or the poly-T insertion can be 8 base pairs corresponding to -TTTTTTTT-. In a particular embodiment, the poly-T insertion is a 7-bp sequence (-TTTTTTT-).

An exemplary haplotype useful in the methods provided herein for assessing level of risk for neurodegenerative disease (e.g., AD), and, in particular for determining protection from Alzheimer's disease, comprises multiple polymorphic regions of the LIPA gene corresponding to nucleotides 1852, 6063 and 7820 of SEQ ID N0:468. In this embodiment, the nucleotide in LIPA at position 1852 of SEQ ID NO:468 is A, at position 6063 of SEQ ID NO:468 is G, and at position 7820 of SEQ ID NO:468 is C.

Individual polymorphisms useful in the methods provided herein for assessing level of risk for neurodegenerative disease (e.g., AD) or determining a predisposition or occurrence of neurodegenerative disease, such as Alzheimer's disease, are also provided herein. Such individual polymorphisms include polymorphisms corresponding to nucleotide positions 122260, 133354/133355 and 132370 of SEQ ID NO:484 and 41014/41015 of SEQ ID N0:347. In particular embodiments of these polymorphisms, the nucleotides are associated with risk for AD and are a G at position 122260, a 7-bp polyT insertion between positions 133354/133355, an A at position 132370, and an insertion of AATTT between positions 41014/41015, or the complements thereof in the complementary positions.

R. Screening Assays for Modulators of uPA, SNCG, IDE, KNSL1, TNFRSF6 or LIPA and Biological Events Characteristic of Diseases and Disorders

Screening methods for identifying (1) molecules or agents that modulate the activity and/or expression of a uPA, SNCG, IDE, KNSL1, TNFRSF6 or LIPA protein, (2) molecules or agents that modulate Aβ protein levels, (3) candidate agents or molecules that modulate a biological event characteristic of neurodegenerative diseases or disorders and (4) candidate agents or molecules that modulate a biological event characteristic of Alzheimer's disease are provided herein. The methods utilize cells and/or animals (in particular, non-human animals) that contain a nucleic acid, either endogenous or heterologous, containing a uPA, SNCG, IDE, KNSL1, TNFRSF6 or LIPA gene or a portion or portions thereof, such as, for example, a transcriptional control region (including, for example, a promoter and 3′ untranslated (UTR) sequences), an intron, and/or a coding sequence, including a cDNA sequence, of a uPA, SNCG, IDE, KNSL1, TNFRSF6 or LIPA gene. The uPA, SNCG, IDE, KNSL1, TNFRSF6 or LIPA gene or portion(s) thereof contains at least one polymorphic region and is thus referred to as a polymorphic uPA, SNCG, IDE, KNSL1, TNFRSF6 or LIPA gene or portion(s) thereof. In particular embodiments, the polymorphic uPA, SNCG, IDE, KNSL1, TNFRSF6 or LIPA gene is a human polymorphic uPA, SNCG, IDE, KNSL1, TNFRSF6 or LIPA gene.

Cells and/or animals used in these methods of identifying modulators that contain endogenous nucleic acid that contains a uPA, SNCG, IDE, KNSL1, TNFRSF6 or LIPA gene or portion(s) thereof that includes one or more polymorphisms of a uPA, SNCG, IDE, KNSL1, TNFRSF6 or LIPA gene can be identified through analysis of nucleic acids, such as the genomic DNA, of the cells or the animal for the presence of the particular nucleotide(s). Methods for the detection of particular polymorphisms are known in the art and are described herein. Cells and/or animals that contain a uPA, SNCG, IDE, KNSL1, TNFRSF6 or LIPA gene or portion(s) thereof that includes one or more polymorphisms of a uPA, SNCG, IDE, KNSL1, TNFRSF6 or LIPA gene as described herein also contain nucleic acid that encodes an endogenous uPA, SNCG, IDE, KNSL1, TNFRSF6 or LIPA protein. Transgenic animals and/or recombinant cells described and provided herein may also be used in the methods of identifying modulators. The transgenic animals and/or recombinant cells contain heterologous nucleic acid that contains a uPA, SNCG, IDE, KNSL1, TNFRSF6 or LIPA gene or portion(s) thereof that includes one or more polymorphisms of a uPA, SNCG, IDE, KNSL1, TNFRSF6 or LIPA gene. Cells which may be used in these methods include recombinant cells described and provided herein as well as cells from the transgenic animals which contain as a heterologous transgenic element nucleic acid that contains a uPA, SNCG, IDE, KNSL1, TNFRSF6 or LIPA gene or portion(s) thereof that includes one or more polymorphisms of a uPA, SNCG, IDE, KNSL1, TNFRSF6 or LIPA gene. The transgenic animals and/or recombinant cells used in these embodiments of the methods may contain nucleic acid that contains the one or more polymorphisms in a sequence or sequences that are operatively linked to nucleic acid encoding a reporter molecule or a uPA, SNCG, IDE, KNSL1, TNFRSF6 or LIPA or other protein or peptide.

In the methods for identifying molecules or agents that modulate the activity and/or expression of a uPA, SNCG, IDE, KNSL1, TNFRSF6 or LIPA protein, molecules or agents that modulate Aβ protein levels, or candidate agents that modulate a biological event characteristic of a disease or disorder, a cell or animal containing a uPA, SNCG, IDE, KNSL1, TNFRSF6 or LIPA gene or portion thereof that includes at least one polymorphic region of a uPA, SNCG, IDE, KNSL1, TNFRSF6 or LIPA gene is combined with a candidate agent. Combining includes any form of contacting the candidate agent with a cell or animal such as, e.g., physical application, injection, oral or intravenous administration, perfusion, addition to surrounding medium and transfection. The affect of the molecule or agent on the expression and/or activity of uPA, SNCG, IDE, KNSL1, TNFRSF6.or LIPA protein expressed by the cell or animal, on the levels of Aβ protein present in the cell and/or surrounding medium or animal, or on a biological event characteristic of a disease or disorder is then assessed.

The agents may be administered to animals, for example, in a variety of ways, orally, topically, parenterally e.g., subcutaneously, intraperitoneally, by viral infection, intravascularly, etc. Oral treatments are of particular interest. Depending upon the manner of introduction, the agents may be formulated in a variety of ways. The concentration of agent in the formulation may vary from about 0.1-100% by weight.

The agents can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Candidate agents encompass numerous chemical classes, though typically they are organic molecules preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs.

1. Screening Methods for Identifying Molecules or Agents that Modulate the Activity and/or Expression of a uPA, SNCG, IDE, KNSL1, TNFRSF6 or LIPA Protein

Molecules or agents that modulate the activity and/or expression of a uPA, SNCG, IDE, KNSL1, TNFRSF6 or LIPA protein can be used for a variety of purposes. For example, such compositions can be used to regulate (a) the activity of the particular proteins, including the enzymatic activities, and/or the activity of serine proteases, (b) the expression of a protein-encoding nucleic acid operatively linked to a uPA, SNCG, IDE, KNSL1, TNFRSF6 or LIPA gene transcription control region, and/or (c) the generation of products resulting from activity of the particular proteins, such as, for example, products of the cleavage of insulin, glucagon, peptide hormones and plasmin, and thus the degradation of fibrin and other protein aggregates such as amyloid Aβ protein.

In a particular embodiment of the methods for identifying molecules or agents that modulate the activity and/or expression of a uPA, SNCG, IDE, KNSL1, TNFRSF6 or LIPA protein, the cells or animals (in particular non-human animals) used in the method contain nucleic acid, either endogenous or heterologous, that contains a uPA, SNCG, IDE, KNSL1, TNFRSF6 or LIPA gene or portion thereof that includes one or more polymorphisms provided herein. In an exemplary embodiment, the one or more polymorphisms occur at a nucleotide position corresponding to the following nucleotide positions in SEQ ID NO: 559 or 560: nucleotide 401 which is an A, T or C; nucleotide 515 which is a T, G or A; nucleotide 748 which is a T, C or A; and nucleotide 1752 which is a C, G or A; in SEQ ID NO: 563 wherein the C nucleotide at position 93 is deleted, and in SEQ ID NO: 563 wherein the nucleotides at positions 714-715 are deleted. In a further particular embodiment, the nucleic acid contains a uPA gene or portion thereof that includes one or more polymorphisms that occur at a nucleotide position corresponding to the following nucleotide positions in SEQ ID NO: 52: nucleotide 401 which is an A; nucleotide 515 which is a T; nucleotide 748 which is a T; and nucleotide 1752 which is a C.

Cells and/or animals used in these embodiments of the method of identifying molecules or agents that modulate the activity and/or expression of a uPA, SNCG, IDE,,KNSL1, TNFRSF6 or LIPA protein contain endogenous nucleic acid that contains a uPA, SNCG, IDE, KNSL1, TNFRSF6 or LIPA gene or portion thereof that includes one or more polymorphisms that occur at the nucleotide position corresponding to the above specified nucleotide positions in SEQ ID NOs: 559, 560 or 563 (uPA); 72 or 73 (SNCG); 186, 187 or 484 (IDE): 347, 348 or 484 (KNSL1); 402 or 403 (TNFRSF6); and 467 or 468 (LIPA)1 can be identified through analysis of the genomic DNA of the cells or the animal for the presence of the particular nucleotide(s). Methods for the detection of particular polymorphisms are known in the art and are described herein. Transgenic animals and/or recombinant cells described and provided herein may also be used in the methods of identifying molecules or agents that modulate the activity and/or expression of a uPA, SNCG, IDE, KNSL1, TNFRSF6 or LIPA protein. The transgenic animals and/or recombinant cells contain heterologous nucleic acid that contains a uPA, SNCG, IDE, KNSL1, TNFRSF6 or LIPA gene or portion thereof that includes one or more polymorphisms that occur at the nucleotide position corresponding to the above specified nucleotide positions in SEQ ID NOs: 559, 560 or 563 (uPA); 72 or 73 (SNCG); 186, 187 or 484 (IDE): 347, 348 or 484 (KNSL1); 402 or 403 (TNFRSF6); and 467 or 468 (LIPA). Cells which may be used in these methods include recombinant cells described and provided herein as well as cells from the transgenic animals which contain as a heterologous transgenic element nucleic acid that contains a uPA, SNCG, IDE, KNSL1, TNFRSF6 or LIPA gene or portion thereof that includes one or more polymorphisms that occur at the nucleotide position corresponding to the above-specified nucleotide positions in SEQ ID NOs: 559, 560 or 563 (uPA); 72 or 73 (SNCG); 186, 187 or 484 (IDE): 347, 348 or 484 (KNSL1); 402 or 403 (TNFRSF6); and 467 or 468 (LIPA). The transgenic animals and/or recombinant cells used in these embodiments of the methods may contain nucleic acid that contains the one or more polymorphisms in a sequence or sequences that are operatively linked to nucleic acid encoding a reporter molecule.

In methods of identifying molecules or agents that modulate the activity of a uPA, SNCG, IDE, KNSL1, TNFRSF6 or LIPA protein, the affect of the candidate molecule or agent on the expression and/or activity of uPA, SNCG, IDE, KNSL1, TNFRSF6 or LIPA protein expressed by the cell or animal can be assessed in a variety of ways. For example, after combining the candidate agent with the cell or animal, the timing and/or level of uPA, SNCG, IDE, KNSL1, TNFRSF6 or LIPA or reporter molecule mRNA expression or the mRNA stability may be evaluated using methods known in the art and described herein for detecting and quantifying mRNA levels in cells. The level of reporter or uPA, SNCG, IDE, KNSL1, TNFRSF6 or LIPA protein and of uPA, SNCG, IDE, KNSL1, TNFRSF6 or LIPA enzymatic and/or binding activity may also be evaluated using methods known in the art and described herein. These measurable aspects of uPA, SNCG, IDE, KNSL1, TNFRSF6 or LIPA or reporter molecule mRNA expression and protein expression and activity can be compared to the same aspects of the mRNA and protein of the same cells and animals in the absence of the candidate agent or to substantially similar cells that contain a different polymorphism (e.g., a wild-type genotype or reference genotype) at the polymorphic site(s) and that have been combined with the agent.

2. Methods of Identifying Molecules or Agents that Modulate Aβ Protein Levels

Also provided herein are methods of identifying agents or molecules that modulate Aβ protein levels. Molecules or agents that modulate Aβ protein levels in cells and the extracellular medium can be used, for example, as candidate agents for the treatment of neurodegenerative diseases that involve deposition of amyloid, such as Alzheimer's disease. In a particular embodiment of the methods for identifying molecules or agents that modulate Aβ protein levels, the cells or animals (in particular non-human animals) used in the method contain nucleic acid, either endogenous or heterologous, that contains a uPA, SNCG, IDE, KNSL1, TNFRSF6 or LIPA gene or portion thereof that includes one or more polymorphisms provided herein. In an exemplary embodiment, the one or more polymorphisms occur at a nucleotide position corresponding to the following nucleotide positions in SEQ ID NO: 559 or 560: nucleotide 401 which is an A, T or C; nucleotide 515 which is a T, G or A; nucleotide 748 which is a T, C or A; and nucleotide 1752 which is a C, G or A; in SEQ ID NO: 563 wherein the C nucleotide at position 93 is deleted, and in SEQ ID NO: 563 wherein the nucleotides at positions 714-715 are deleted. In a further particular embodiment, the nucleic acid contains a uPA gene or portion thereof that includes one or more polymorphisms that occur at a nucleotide position corresponding to the following nucleotide positions in SEQ ID NO: 52: nucleotide 401 which is an A; nucleotide 515 which is a T; nucleotide 748 which is a T; and nucleotide 1752 which is a C.

In another embodiment of the methods for identifying molecules or agents that modulate Aβ protein levels, the cells or animals used in the method contain nucleic acid, either endogenous or heterologous, that contains a uPA gene or portion thereof that includes one or more polymorphisms that occur at a nucleotide position corresponding to the following nucleotide positions in SEQ ID NO: 559 or 560: 3169, 3947 and 6532. In a particular embodiment of this embodiment, the nucleotide in position 3169 is T, at position 3947 is C, and at position 6532 is T.

In a further embodiment of the methods for identifying molecules or agents that modulate Aβ protein levels, the cells or animals used in the method contain nucleic acid, either endogenous or heterologous, that contains a uPA gene or portion thereof that includes one or more polymorphisms that occur at a nucleotide position corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 in SEQ ID NO:563. In yet a further embodiment of the methods of identifying molecules or agents that modulate Aβ protein levels, the cells or animals used in the method contain nucleic acid, either endogenous or heterologous, that contains a uPA gene or portion thereof that includes one or more polymorphisms that occur at a nucleotide position corresponding to the following nucleotide positions of SEQ ID NO: 559 or 560: 9, 401, 464, 515, 748, 1229, 1356, 1752, 1942, 2127, 2543, 3029, 3169, 3799, 3947, 4808, 5287, and 6532. In a another embodiment, a cell or animal used in the method contains nucleic acid that contains a uPA gene or portion thereof that includes one or more polymorphisms that occurs at a nucleotide position corresponding to the following nucleotide positions of SEQ ID NO: 559 or 560: 9, 401, 464, 515, 748, 1229, 1356, 1752, 1942, 2127, 2543, 3029 and 5287.

In another embodiment, a cell or animal used in the methods of identifying a molecule or agent that modulates Aβ protein levels provided herein contains nucleic acid containing a uPA gene or portion thereof that includes one or more polymorphisms that occurs at a nucleotide position corresponding to the following nucleotide positions: 178, 1363, 1423, 1465, 1540, 2297,.2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotide positions 79, 93, 256, 385 and 714 of SEQ ID NO:563. In yet another embodiment, a cell or animal used in the methods provided herein contains nucleic acid containing a uPA gene or portion thereof that includes one or more polymorphisms that occurs at a nucleotide position corresponding to the following nucleotide positions: 178, 401, 464, 515 and 748 in SEQ ID NO: 559 or 560 and positions 79, 93, 256, 385 and 714 of SEQ ID NO:563. In a further embodiment, a cell or animal used in the methods provided herein contains nucleic acid containing a uPA gene or portion thereof that includes one or more polymorphisms that occurs at a nucleotide position corresponding to the following nucleotide positions of SEQ ID NO: 559 or 560: 401, 515 and 748. In a further embodiment, a cell or animal used in the methods of identifying a molecule or agent that modulates Aβ protein levels provided herein contains nucleic acid containing a uPA gene or portion thereof that includes one or more polymorphisms that occurs at a nucleotide position corresponding to the following nucleotide positions of SEQ ID NO: 559 or 560: 6519, 6532, 6909 and 7235.

In particular embodiments of any of the above embodiments of the methods of identifying a molecule or agent that modulates Aβ protein levels provided herein, the nucleotide at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, at position 6532 is T or C, at position 178 is A or G., at position 1363 is C or A, at position 1423 is G or T, at position 1465 is C or A, at position 1540 is C or T, at position 2297 is C or T, at position 2445 is T or G, at position 2653 is G or A, at position 3080 is G or A, at position 3546 is C or G, at position 3664,is C or T, at position 3816 is A or C, at position 4320 is T or C, at position 4369 is G or A, at position 4399 is C or A, at position 4851 is G or A, at position 5186 is G or A, at position 5204 is G or A, at position 5787 is C or G, at position 6519 is C or G, at position 6909 is G or T, at position 7235 is G or position 7235 is deleted, at position 7848 is C or T, at position 7908 is A or C; and the nucleotide in SEQ ID NO:563: at position 79 is T or C, at position 93 is a C or position 93 is deleted, at position 256 is G or T, at position 385 is C or T, at position 714-715 is the dinulceotide -GT- or the -GT- dinucleotide is deleted.

In a further embodiment of the methods for identifying molecules or agents that modulate Aβ protein levels, the cells or animals used in the method contain nucleic acid, either endogenous or heterologous, that contains an IDE gene or portion thereof that includes one or more polymorphisms that occur at a nucleotide position corresponding to the following nucleotide positions: IDE nucleotide positions 2456, 3279, 3407,42943, 62498, 69586, 107395, 112114, 116662, 17095, 17242, 33590, 38903, 43391, 45017, 68906,68973, 73772, 74084, 83024, 83104, 89301, 105060, 108489, 111914, 113142, 113591, 114683, 117803 and 124565 of SEQ ID NO:187; the complement of IDE nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124 of SEQ ID NO:484.

In particular embodiments of any of the above embodiments of the methods of identifying a molecule or agent that modulates Aβ protein levels provided herein, the IDE nucleotide at position 2456 is T or G, at position 3279 is T or C, at position 3407 is C or T, at position 42943 is T or C, at position 62498 is T or C, at position 69586 is T or C, at position 107395 is G or A, at position 112114 is G or A, and at position 116662 is T or A; and wherein the complementary nucleotide in SEQ ID NO:484 at position 820 is A or T, at position 7066 is A or G, at position 11758 is T or C, at position 21270 is T or G, at position 22225 is A or T, at position 29294 is C or T, at position 33452 is G or T, at position 33708 is G or A, at position 36982 is C or T, at position 54862 is A or G, at position 77786 is C or A, at position 80594 is G or A, at position 84792 is T or C, at position 84997 is G or T, at position 86682 is C or T, at position 86857 is T or A, at position 88511 is A or G, at position 90437 is G or T, at position 90593 is G or A, at position 91650 is T or C, at position 91870 is G or A, at position 91878 is G or A, at position 92011 is C or T, at position 9361 8 is T or C, at position 94344 is C or T, at position 94714 is A or G, at position 95671 is A or G, at position 96324 is A or G, at position 97302 is G or A, at position 97370 is G or A, at position 98253 is T or C, at position 98276 is C or T, at position 98385 is A or G, at position 98646 is T or A, at position 98814 is G or A, at position 99597 is C or T, at position 100378 is T or C, at position 101029 is G or A, at position 101265 is C or T, at position 102465 is C or G, at position 103289 is T or G, at position 103967 is C or T, at position 105793 is A or G, at position 106076 is G or T, at position 106453 is C or T, at position 106600 is A or G, at position 106995 is G or A, at position 107851 is C or T, at position 108434 is G or C, at position 109096 is C or T, at position 109399 is C or T, at position 109483 is T or G, at position 110870 is G or A, at position 111189 is A or G, at position 111972 is G or A, at position 112627 is A or T, at position 112629 is A or T, at position 112631 is T or A, at position 113407 is C or G, at position 114444 is C or G, at position 114482 is G or C, at position 115473 is C or position 115473 is deleted, at position 116681 is G or T, at position 117226 is A or T, at position 117600 is A or G, at position 117802 is C or T, at position 11 8223 is G or C, at position 120011 is C or T, at position 122260 is A or G, at position 123165 is A or G, at position 123424 is G or A, at position 124352 is A or G, at position 124501 is C or T, at position 124692 is A or G, at position 125113 is T or A, at position 125159 is G or A, at position 126568 is G or C, at position 127166 is C or G, at position 127598 is T or C, at position 127600 is T or C, at position 127609 is T or C, at position 127614 is T or C, at position 127623 is T or C, at position 127662 is G or A, at position 128053 is G or A, at position 128261 is a repeat of -TAAA- occurring 6, 7, or 8 times beginning at position 128261, at position 128289 is A or T, at position 128291 is T or G, at position 128393 is T or G, at position 129444 is C or T.

In another embodiment of the methods for identifying molecules or agents that modulate Aβ protein levels, the cells or animals used in the method contain nucleic acid, either endogenous or heterologous, that contains an IDE gene or portion thereof that includes one or more polymorphisms that occur at a nucleotide position corresponding to the nucleotide positions 2456, 3279, 3407 and 42943 of SEQ ID NO:187. In a particular embodiment of this embodiment, the nucleotide the nucleotide in IDE at position 2456 of SEQ ID NO:187 is G, at position 3279 of SEQ ID NO:187 is T, at position 3407 of SEQ ID NO:187 is T, and at position 42943 of SEQ ID NO:187 is T. In another embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is T. In another embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is C. In yet another embodiment, the nucleotide in IDE at position 2456 of SEQ ID NO:187 is T, at position 3279 of SEQ ID NO:187 is C, at position 3407 of SEQ ID NO:187 is C, and at position 42943 of SEQ ID NO:187 is C.

In methods of identifying molecules or agents that modulate Aβ protein levels, the affect of the candidate molecule or agent on Aβ protein levels in the cell, extracellular medium or animal can be assessed using a number of assays known in the art for quantifying Aβ, for example, immunological assays. Thus, for example, Aβ protein levels measured in a cell, extracellular medium or animal in the presence and absence of the candidate agent can be compared in determining the affect of the agent on the protein levels. Aβ protein levels may also be compared in cells or animals that have been combined with the agent and that are substantially similar except that the control cell or animal to which the screening assay cell or animal is being compared either does not contain nucleic acid containing a uPA, SNCG, IDE, KNSL1, TNFRSF6 or LIPA gene or portion thereof or contains nucleic acid that contains a uPA, SNCG, IDE, KNSL1, TNFRSF6 or LIPA gene or portion thereof that does not possess the same particular polymorphisms that the assay cell or animal possesses.

In particular methods of identifying molecules or agents that modulate Aβ protein levels as provided herein, the cell or animal used in the method contains endogenous or heterologous nucleic acid that provides for increased expression of Aβ protein in the cell and/or extracellular medium or animal relative to a similar cell or animal that does not contain the nucleic acid. For example, such a nucleic acid may be one that encodes amyloid precursor protein.

Aβ levels can be assessed, for example, in cells, tissues, body fluids or extracellular medium using methods known in the art, such as ELISA assays (e.g. sandwich ELISA assays using two different Aβ antibodies), or immunoprecipitation followed by gel electrophoresis or mass spectroscopy.

3. Methods of Identifying Molecules or Agents that Modulate a Biological Event and/or Behavioral Phenomenon Characteristic of Neurodegenerative Diseases or Disorders

Also provided herein are methods of identifying agents or molecules that modulate a biological event and/or behavioral phenomenon characteristic of a neurodegenerative disease or disorder. The term “biological event” encompasses all physiological and behavioral phenomena characteristic of the disease or disorder. These methods provide a system for screening for ligands or substrates that modulate phenomena associated with a neurodegenerative disease or disorder. Molecules or agents that modulate a biological event characteristic of a neurodegenerative disease or disorder can be used, for example, as candidate agents for the treatment of neurodegenerative diseases and disorders. Of particular interest are screening assays for agents that have a low toxicity for human cells. For example, therapeutic peptides, peptidomimetics, or small molecules may be used to delay onset, lessen symptoms, or halt or delay progression of a neurodegenerative disease or disorder. Thus, the term “agent” as used herein with respect to methods of identifying agents that modulate a biological event and/or behavioral phenomenon characteristic of a disease or disorder is meant to include any molecule, e.g., protein or pharmaceutical, with the capability of affecting the molecular and clinical phenomena associated with a disease or disorder. Generally a plurality of assay mixtures are run in parallel with different agent concentrations to obtain a differential response to the various concentrations. Typically, one of these concentrations serves as a negative control, ire. at zero concentration or below the level of detection.

Characteristics of neurodegenerative disease which have been widely described and are known to those of skill in the art, are numerous and include morphological, structural, biological and biochemical occurrences which can be pathophysiological aspects of neurodegenerative diseases. Such phenomena include, but are not limited to, senile plaques, neuritic plaques, and components of each, neurofibrillary tangles, tau protein and abnormal phosphorylation of tau protein, amyloid precursor protein (APP) and processing thereof, Aβ42 protein, α-, β- and γ-secretases, presenilin proteins, amyloid deposition, Lewy bodies, prions, apoptosis (see, e.g., Behl (2000) J. Neural Transm. 107:1325-1344), caspases, inflammation (see, e.g., McGeer and McGeer (1998) Exp. Gerontol. 33:371-378), excitotoxicity and excitotoxins, excessive nitric oxide production, oxidative stress (see, e.g., Beal (1998) Biochim. Biophys. Acta Mol. Cell Res. 1366:211-223, and Wallace et al. (1998) Biofactors 7:187-190), proteases, protease inhibitors, neurotrophic factors, cytokines, calcium-dependent processes, signal transduction, altered ionic homeostasis, particularly calcium homeostasis, synaptic molecules, adhesion molecules, molecules involved in membrane turnover, cholesterol and lipid metabolism and transport, cytoskelet al molecules, neuronal and brain proteins, and cell necrosis. These characteristics may be assessed in the screening assays either singly or in any combination.

When animals are used in the methods of identifying a candidate molecule or agent that modulates a biological event and/or behavioral phenomenon characteristic of a neurodegenerative disease, the affect of the agent on behavioral phenomena associated with neurodegenerative disease, such as memory or learning deficits, may be assessed. For example, memory and learning can be tested in rodents by the Morris water maze (Stewart and Morris (1993) “Behavioral Neuroscience,” IRL Press, R. Saghal ed. 107) and the Y-maze (Brits et al. (1981) Brain Res. Bull. 6:71). In these methods, the agent or molecule is administered to animals. The response time in trials is measured and an improvement in memory and learning is demonstrated by a statistically significant decrease in the timed trials.

In a particular embodiment of the methods for identifying candidate molecules or agents that modulate a biological event or behavioral phenomenon characteristic of neurodegenerative disease, the cells or animals (in particular non-human animals) used in the method contain nucleic acid, either endogenous or heterologous, that contains a uPA, SNCG, IDE, KNSL1, TNFRSF6 or LIPA gene or portion thereof that includes one or more polymorphisms provided herein. In an exemplary embodiment, ,the one or more polymorphisms occur at a nucleotide position corresponding to the following nucleotide positions in SEQ ID NO: 559 or 560: nucleotide 401 which is an A, T or C; nucleotide 515 which is a T, G or A; nucleotide 748 which is a T, C or A; and nucleotide 1752 which is a C, G or A; in SEQ ID NO: 563 wherein the C nucleotide at position 93 is deleted, and in SEQ ID NO: 563 wherein the nucleotides at positions 714-715 are deleted. In a further particular embodiment, the nucleic acid contains a uPA gene or portion thereof that includes one or more polymorphisms that occur at a nucleotide position corresponding to the following nucleotide positions in SEQ ID NO: 52: nucleotide 401 which is an A; nucleotide 515 which is a T; nucleotide 748 which is a T; and nucleotide 1752 which is a C.

In another embodiment of the methods for identifying candidate molecules or agents that modulate a biological event or behavioral phenomenon characteristic of neurodegenerative disease, the cells or animals used in the method contain nucleic acid, either endogenous or heterologous, that contains a uPA gene or portion thereof that includes one or more polymorphisms that occur at a nucleotide position corresponding to the following nucleotide positions in SEQ ID NO: 559 or 560: 3169, 3947 and 6532. In a particular embodiment of this embodiment, the nucleotide in position 3169 is T, at position 3947 is C, and at position 6532 is T.

In a further embodiment of the methods for identifying candidate molecules or agents that modulate a biological event or behavioral phenomenon characteristic of neurodegenerative disease, the cells or animals used in the method contain nucleic acid, either endogenous or heterologous, that contains a uPA gene or portion thereof that includes one or more polymorphisms that occur at a nucleotide position corresponding to the following nucleotide positions: nucleotide 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotides 79, 93, 256, 385 and 714 in SEQ ID NO:563. In yet a further embodiment of the methods of identifying candidate molecules or agents that modulate a biological event or behavioral phenomenon characteristic of neurodegenerative disease, the cells or animals used in the method contain nucleic acid, either endogenous or heterologous, that contains a uPA gene or portion thereof that includes one or more polymorphisms that occur at a nucleotide position corresponding to the following nucleotide positions of SEQ ID NO: 559 or 560: 9, 401, 464, 515, 748, 1229, 1356, 1752, 1942, 2127, 2543, 3029, 3169, 3799, 3947, 4808, 5287, and 6532. In a another embodiment, a cell or animal used in the method contains nucleic acid that contains a uPA gene or portion thereof that includes one or more polymorphisms that occurs at a nucleotide position corresponding to the following nucleotide positions of SEQ ID NO: 559 or 560: 9, 401, 464, 515, 748, 1229, 1356, 1752, 1942, 2127, 2543, 3029 and 5287.

In another embodiment, a cell or animal used in the methods of identifying a candidate molecule or agent that modulates a biological event or behavioral phenomenon characteristic of neurodegenerative disease provided herein contains nucleic acid containing a uPA gene or portion thereof that includes one or more polymorphisms that occurs at a nucleotide position corresponding to the following nucleotide positions: 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848 and 7908 in SEQ ID NO: 559 or 560 and nucleotide positions 79, 93, 256, 385 and 714 of SEQ ID NO:563. In yet another embodiment, a cell or animal used in the methods provided herein contains nucleic acid containing a uPA gene or portion thereof that includes one or more polymorphisms that occurs at a nucleotide position corresponding to the following nucleotide position: 178, 401, 464, 515 and 748 in SEQ ID NO: 559 or 560 and positions 79, 93, 256, 385 and 714 of SEQ ID NO:563. In a further embodiment, a cell or animal used in the methods provided herein contains nucleic acid containing a uPA gene or portion thereof that includes one or more polymorphisms that occurs at a nucleotide position corresponding to the following nucleotide positions of SEQ ID NO: 559 or 560: 401, 515 and 748. In a further embodiment, a cell or animal used in the methods of identifying a candidate molecule or agent that modulates a biological event or behavioral phenomenon characteristic of neurodegenerative disease provided herein contains nucleic acid containing a uPA gene or portion thereof that includes one or more, polymorphisms that occurs at a nucleotide position corresponding to the following nucleotide positions of SEQ ID NO: 559 or 560: 6519, 6532, 6909 and 7235.

In particular embodiments of any of the above embodiments of the methods of identifying a candidate molecule or agent that modulates a biological event or behavioral phenomenon characteristic of neurodegenerative disease provided herein, the nucleotide at position 401 is G or A, at position 464 is G or position 464 is deleted, at position 515 is C or T, at position 748 is G or T, at position 1229 is T or G, at position 1356 is C or T, at position 1752 is T or C, at position 1942 is G or A, at position 2127 is G or A, at position 2543 is G or A, at position 3029 is G or A, at position 3169 is C or T, at position 3799 is T or C, at position 3947 is C or T, at position 4808 is C or T, at position 5287 is T or C, at position 6532 is T or C, at position 178 is A or G, at position 1363 is C or A, at position 1423 is G or T, at position 1465 is C or A, at position 1540 is C-or T, at position 2297 is C or T, at position 2445 is T or G, at position 2653 is G or A, at position 3080 is G or A, at position 3546 is C or G, at position 3664 is C or T, at position 3816 is A or C, at position 4320 is T or C, at position 4369 is G or A, at position 4399 is C or A, at position 4851 is G or A, at position 5186 is G or A, at position 5204 is G or A, at position 5787 is C or G, at position 6519 is C or G, at position 6909 is G or T, at position 7235 is G or position 7235 is deleted, at position 7848 is C or T, at position 7908 is A or C; and the nucleotide in SEQ ID NO:563: at position 79 is T or C, at position 93 is a C or position 93 is deleted, at position 256 is G or T, at position 385 is C or T, at position 714-715 is the dinulceotide -GT- or the -GT- dinucleotide is deleted.

Also provided are methods of identifying a candidate molecule or agent that modulates a biological event or behavioral phenomenon characteristic of neurodegenerative disease in which the cell or animal used in the methods contains nucleic acid containing a uPA, SNCG, IDE, KNSL1, TNFRSF6 or LIPA gene or portion thereof that includes one or more polymorphisms that are associated, individually and/or in combination, with a neurodegenerative disease or disorder. In particular embodiments of these methods, the cell or animal used in the method contains nucleic acid containing a uPA, SNCG, IDE, KNSL1, TNFRSF6 or LIPA gene or portion thereof that includes one or more polymorphisms that are associated, individually and/or in combination, with a neurodegenerative disease or disorder and that occurs at the above-specified nucleotide positions.

Further provided are any of the above methods for identifying a candidate molecule or agent that modulates a biological event or behavioral phenomenon characteristic of neurodegenerative disease wherein the neurodegenerative disease is Alzheimer's disease.

In particular methods of identifying candidate molecules or agents that modulate a biological event and/or behavioral phenomenon characteristic of a neurodegenerative disease, e.g., Alzheimer's disease, as provided herein, the cell or animal used in the method contains endogenous or heterologous nucleic acid that provides for increased expression of Aβ protein in the cell and/or extracellular medium or animal relative to a similar cell or animal that does not contain the nucleic acid. For example, such a nucleic acid may be one that encodes amyloid precursor protein.

In methods of identifying candidate molecules or agents that modulate a biological event or behavioral phenomenon characteristic of neurodegenerative disease, the affect of the candidate molecule or agent on a characteristic of a neurodegenerative disease may be assessed in a variety of ways, depending on the characteristic being evaluated. For example, the affect of a candidate agent on apoptosis, nitric oxide production, oxidative stress, proteases activity, calcium-dependent processes, signal transduction, ionic homeostasis, particularly calcium homeostasis, synaptic molecules, adhesion molecules, molecules involved in membrane turnover, cholesterol and lipid metabolism and transport, cytoskelet al molecules, neuronal and brain proteins, and/or cell necrosis may be assessed and compared in cells and/or animals in the presence and absence of the candidate agent. Numerous techniques for assessing such phenomena are known in the art. In addition, when animals are used in the methods, behavioral phenomena and tissue, in particular brain tissue, may be assessed and compared in the presence and absence of candidate agent. In methods of identifying candidate molecules or agents that modulate a biological event and/or behavioral phenomenon characteristic of Alzheimer's disease, amyloid deposition and clearance, as well as memory and learning capacity may particularly be evaluated in determining the affect of the agent on a characteristic of Alzheimer's disease. Depending on the particular assay, whole animals may be used, or cells derived therefrom. Cells may be freshly isolated from an animal, or may be immortalized in culture. Cells of particular interest are derived from neural tissue. For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

A number of assays are known in the art for determining the affect of a drug on animal behavior and other phenomena associated with a neurodegenerative disease, such as AD. Some examples are provided, although it will be understood by one of skill in the art that many other assays may also be used. The subject animals may be used by themselves, or in combination with control animals. Control animals may have, for example, a wild-type transgene that is not associated with AD.

The screen using transgenic animals can employ any phenomena associated with AD that can be readily assessed in an animal model. The screening for AD can include assessment of phenomena including, but not limited to: 1) analysis of molecular markers (e.g., levels of expression of APP gene products in brain tissue; presence/absence in brain tissue of various APP splice variants, isoforms, and mutants associated with AD; and formation of neurite plaques); 2) an enzyme, inhibitor or regulatory subunit in a pathway leading to the generation of a component of an amyloid plaque, e.g., Aβ, alpha-1-anti-chymotrypsin, cathepsin D, non-amyloid component protein, apolipoprotein E (APOE), apolipoprotein J, heat shock protein 70, complement components, alpha2-macroglobin, interleukin-6, proteoglycans and serum amyloid P; 3) assessment of behavioral symptoms associated with memory and learning; 4) detection of neurodegeneration characterized by progressive and irreversible deafferentation of the limbic system, association neocortex, and basal forebrain (neurodegeneration can be measured by, for example, detection of synaptophysin expression in brain tissue) (see, e.g., Games et al. (1995) Nature 373:523-7). These phenomena may be assessed in the screening assays either singly or in any combination.

Preferably, the screen will include control values (e.g., the level of amyloid production in the test animal in the absence of test compound(s)). Test substances which are considered positive, ice., likely to be beneficial in the treatment of AD, will be those which have a -substantial affect upon an AD-associated phenomenon.

Methods for assessing these phenomena, and the affects expected of a candidate agent for treatment of AD are well known in the art. For example, methods for using transgenic animals in various screening assays for, for example, testing compounds for an affect on AD, are found in WO 9640896, published Dec. 19, 1996; WO 9640895, published Dec. 19, 1996; WO 9511994, published May 4, 1995 (describing methods and compositions for in vivo monitoring of A-beta; each of which is incorporated herein by reference with respect to disclosure of methods and compositions for such screening assays and techniques). Examples of assessment of these phenomena are provided below, but are not meant to be limiting.

As set forth herein, through use of the subject transgenic animals, cells derived therefrom, or cells in which nucleic acids encoding allelic variants have been introduced, one can identify ligands or substrates that modulate phenomena associated with neurodegenerative diseases, including AD, e.g., amyloid deposition, neurodegeneration, and/or behavioral phenomena, etc. Of particular interest are screening assays for agents that have a low toxicity for human cells.

Therapeutic peptides, peptidomimetics, or small molecules may be used to delay onset of neurodegenerative disease, lessen symptoms, or halt or delay progression of the disease. Such therapeutics may be tested in a transgenic animal model that expresses mutant protein, wild-type and mutant protein, or in an in vitro assay system. In addition, transgenic animal models and in vitro assay systems can utilize polymorphisms that are not located in the protein coding region of uPA, SNCG, IDE, KNSL1, TNFRSF6 or LIPA genes but affect the expression of transcription, translation, RNA processing or RNA stability.

One such in vitro assay system measures the amount or activity of uPA, SNCG, IDE, KNSL1, TNFRSF6 or LIPA protein produced. Briefly, by way of illustration, a cell expressing mutant uPA, mutant SNCG, mutant IDE, mutant LIPA, mutant TNFRSF6, and/or mutant KNSL1 is cultured in the presence of a candidate therapeutic molecule. The protein expressed by the cell may be either wild-type or mutant protein. In either case, the amount of protein that is produced is measured from cells incubated with or without (control) the candidate therapeutic. Briefly, by way of example, cells are labeled in medium containing ³⁵S-methionine and incubated in the presence (or absence) of candidate therapeutic. Protein is detected in the culture supernatant by immunoprecipitation and SDS-PAGE electrophoresis or by ELISA. A statistically significant reduction of the amount or activity of the protein compared to the control signifies a therapeutic suitable for use in preventing or treating Alzheimer's disease.

Alternatively, transgenic animals expressing Alzheimer's disease protein may be used to test candidate therapeutics. uPA, SNCG, IDE, LIPA, TNFRSF6 or KNSL1 protein is measured or, if the animals exhibit other disease symptoms, such as memory or learning deprivation, an increase in memory or learning is measured. Memory and learning are tested in rodents by the Morris water maze (Stewart and Morris (1993) “Behavioral Neuroscience,” IRL Press, R. Saghal ed. 107) and the Y-maze (Brits et al. (1981) Brain Res. Bull. 6:71). Therapeutics are administered to animals prior to testing. The response time in trials are measured and an improvement in memory and learning is demonstrated by a statistically significant decrease in the timed trials.

A wide variety of assays may be used for this purpose, including behavioral studies, determination of the localization of relevant proteins after administration, immunoassays to detect amyloid deposition, and the like. Depending on the particular assay, whole animals may be used, or cells derived therefrom. Cells may be freshly isolated from an animal, or may be immortalized in culture. Cells of particular interest are derived from neural tissue.

The term “agent” as used herein describes any molecule, e.g., protein or pharmaceutical, with the capability of affecting the molecular and clinical phenomena associated with AD or neurodegenerative disease. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs.

For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

A number of assays are known in the art for determining the affect of a drug on animal behavior and other phenomena associated with AD. Some examples are provided, although it will be-understood by one of skill in the art that many other assays may also be used. The subject animals may be used by themselves, or in combination with control animals. Control animals may have, for example, a wild-type transgene that is not associated with AD.

Pathological Studies

After exposure to the candidate agent, the animals are sacrificed and analyzed by immunohistology for either: 1) neuritic plaques and neurofibrillary tangles (NFTs) in the brain (AD model) and/or 2) amyloid deposition on cerebrovascular walls (CAA). The brain tissue is fixed (e.g., in 4% paraformaldehyde) and sectioned; the sections are stained with antibodies reactive with the APP and/or the beta peptide. Secondary antibodies conjugated with fluorescein, rhodamine, horseradish peroxidase, or alkaline phosphatase are used to detect the primary antibody. These experiments permit identification of amyloid plaques and the regionalization of these plagues to specific areas of the brain.

Sections are also stained with other antibodies diagnostic of Alzheimer's plaques, recognizing antigens such as Alz-50, tau, A2B5, neurofilaments, neuron-specific enolase, and others that are characteristic of Alzheimer's plaques. Staining with thioflavins and congo red can also be carried out to analyze co-localization of A-beta deposits within the neuritic plaques and NFTs of AD.

APP and A-beta expression can also be analyzed by a variety of methods. Messenger RNA (mRNA) can be isolated by the acid guanidinium thiocyanate phenol:chloroform extraction method (Chomczynski et al. (1987) Anal Biochem 162:156-159) from cell lines and tissues of transgenic animals to determine expression levels by Northern blots.

Radioactive or enzymatically labeled probes can be used to detect mRNA in situ. The probes are degraded approximately to 100 nucleotides in length for better penetration of cells. The procedure of Chou et al. (1990) J Psychiatr Res 24:27-50 for fixed and paraffin embedded samples is briefly described below although similar procedures can be employed with samples sectioned as frozen material. Paraffin slides for in situ hybridization are dewaxed in xylene and rehydrated in a graded series of ethanols and finally rinsed in phosphate buffered saline (PBS). The sections are postfixed in fresh 4% paraformaldehyde. The slides are washed with PBS twice for 5 minutes to remove paraformaldehyde. Then the sections are permeabilized by treatment with a 20 μg/ml proteinase K solution. The sections are refixed in 4% paraformaldehyde, and basic molecules that could give rise to background probe binding are acetylated in a 0.1M triethanolamine, 0.3M acetic anhydride solution for 10 minutes. The slides are washed in PBS, then dehydrated in a graded series of ethanols and air dried. Sections are hybridized with antisense probe, using sense probe as a control. After appropriate washing, bound radioactive probes are detected by autoradiography or enzymatically labeled probes are detected through reaction with the appropriate chromogenic substrates.

Western Blot Analysis: Protein fractions can be isolated from tissue homogenates and cell lysates and subjected to Western blot analysis as described by Harlow et al. (1988) “Antibodies: A laboratory manual,” (Cold Spring Harbor, N.Y.); Brown et al. (1983) J. Neurochem 40:299-308; and Tate-Ostroff et al. (1989) Proc Natl Acad Sci 86:745-749. The protein fractions can be denatured in Laemmli sample buffer and electrophoresed on SDS-polyacrylamide gels. The proteins are then transferred to nitrocellulose filters by electroblotting. The filters are blocked, incubated with primary antibodies, and finally reacted with enzyme conjugated secondary antibodies. Subsequent incubation with the appropriate chromogenic substrate reveals the position of APP proteins.

Behavioral Studies of Transgenic Mice and Rats

Behavioral tests designed to assess learning and memory deficits can be employed in methods that involve assessing the effect of a test agent on a biological event characteristic of a neurodegenerative disease. An example of such as test is the Morris Water maze (Morris (1981) Learn Motivat 12:239-260). In this procedure, the animal is placed in a circular pool filled with water, with an escape platform submerged just below the surface of the water. A visible marker is placed on the platform so that the animal can find it by navigating toward a proximal visual cue. Alternatively, a more complex form of the test in which there are no formal cues to mark the platform's location will be given to the animals. In this form, the animal must learn the platform's location relative to distal visual cues.

Alternatively, or in addition, memory and learning deficits can be studied using a 3 runway panel for working memory impairment (attempts to pass through two incorrect panels of the three panel-gates at four choice points) (Ohno et al. (1997) Pharmacol Biochem Behav 57:257-261).

In addition to the use of transgenic animals and cells, modulators can be screened or identified using methods familiar to those skilled in the art, such as in silico drug discovery procedures and rational drug design methodology.

uPA, SNCG, IDE, KNSL, TNFRSF6 and LIPA Functions

In the screening methods described herein, the expression of uPA, SNCG, IDE, KNSL1, TNFRSF6 and/or LIPA coding sequence can be assessed by monitoring the level of protein activity based on one or more the functions described herein.

SNCG

Gamma-synuclein (SNCG) increases the susceptibility of neurofilament-H to calcium-dependent proteases, and may participate in the regulation of neurofilament network integrity (Buchman et al. (1998) Nat Neurosci 1(2): 101-103). Thus, assays directed toward measuring intact versus degraded neurofilament-H can be used to determine and monitor the function of a particular SNCG allelic variant.

IDE

Insulin-degrading enzyme (IDE) is a thiol metalloendopeptidase known to cleave amyloid-beta peptides, insulin, glucagon, atrial naturitic peptide, other peptide hormones (see, for example, Qui et al. (1998) J. Biol. Chem. 4:32730-32738). Thus, assays directed toward measuring the cleavage of insulin, glucagon, and other peptide hormones can be used to determine and monitor the function of a particular IDE allelic variant. IDE activity assays can involve the step of measuring the cleavage of an IDE substrate, or of a synthetic peptide containing amino acids flanking the cleavage site of an IDE substrate. For example, an IDE substrate sequence can be flanked by a donor fluorescent moiety and an acceptor quencher moiety. The uncleaved substrate will have low fluorescence due to quenching of the donor by the acceptor. Upon cleavage by IDE, the donor and acceptor moieties are no longer in proximity, and the donor fluoresces strongly because it is no longer quenched. The amount of donor fluorescence is directly proportional to the IDE activity. Other assays for determining peptidase activity are well known in the art and can be adapted for use in assessing IDE activity.

KNSL

KNSL1 (also referred to as human Eg5) has been shown to be required for proper assembly and dynamics of the mitotic spindle and for proper mitosis function. Thus, assays directed toward measuring mitotic spindle assembly and centrosome migration function can be used to determine and monitor the function of a particular KNSL1 allelic variant. See, e.g., Whitehead et al. (1998) J. Cell Sci. 111(17):2551-61; and Blangy etal. (1995) Cell 83(7):1159-69. KNSL1 is also an ATPase. Thus, KNSL1 activity assays can involve the step of measuring ATP hydrolysis. An exemplary ATP hydrolysis assay suitable for determining KNSL1 activity is described in Maliga et al. (2002) Chemistry & Biology 9:989-996, and involves an enzyme-coupled system that regenerates ADP to ATP, with NADH fluorescence serving as an indirect measure of ATP turnover. Another activity of KNSL1 is binding to microtubules. Thus, KNSL1 activity assays can involve the step of measuring binding of KNSL1 to microtubules (e.g. Woehlke et al. (1997) Cell 90:207-216).

TNFRSF6

TNFRSF6 mediates apoptosis (programmed cell death). Any assay for examining apoptosis or components of such a pathway can be utilized to determine and monitor the function of a particular TNFRSF6 allelic variant. Other useful assays include binding of Fas to the TNFRSF6 ligand binding domain, functioning of the TNFRSF6 death domain, e.g., binding of fadd, formation of the death-inducing signaling complex of TNFRSF6, fadd and caspase-8, caspase-8 proteolytic activation, and monitoring induction of peripheral tolerance or antigen stimulated suicide of mature T cells.

LIPA

LIPA is a triacylglycerol lipase and a cholesteryl esterase. Assays directed toward measuring acid lipase/cholesteryl ester hydrolase activity, intralysosomal lipid accumulations of cholesterol esters and triglycerides and cholesterol production (the affect of low density lipoprotein (LDL) uptake on the suppression of hydroxymethylglutaryl-CoA reductase and activation of endogenous cellular cholesteryl ester formation (Brown et al. (1976) J. Biol. Chem. 251:3277-3286)) can be used to determine and monitor the function of a particular LIPA allelic variant. For example, to measure enzyme activity, white blood cells from liver biopsies or cultured skin fibroblasts can be incubated in the presence of ¹⁴C-trioleylglycerol and the release of radioactivity from the compound is measured.

Therapeutic Agents Identified Using the Transgenic Animals

The therapeutic agents may be administered in a variety of ways, orally, topically, parenterally e.g., subcutaneously, intraperitoneally, by viral infection, intravascularly, etc. Oral treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Thus, depending on the affect of the alteration, a specific treatment can be administered to a subject having such a mutation. For example, if the mutation results in decreased production of a protein, the subject can be treated by administration of a compound which increases synthesis, such as by increasing gene expression. Alternatively, if the mutation results in increased protein, the subject can be treated by administration of a compound which reduces protein production, e.g., by reducing gene expression or a compound which inhibits or reduces the activity of a protein.

S. PHARMACOGENOMICS

It is likely that subjects having one or more different allelic variants of the uPA, SNCG, IDE, LIPA, TNFRSF6 or KNSL1 polymorphic regions will respond differently to drugs to treat neurodegenerative disease. Alleles of the uPA, SNCG, IDE, LIPA, TNFRSF6 or KNSL1 genes that associate with neurodegenerative disease will be useful alone or in conjunction with other genes associated with the development of neurodegenerative disease (e.g., APOE4) to predict a subject's response, either positive or negative, to a therapeutic drug. Multiplex primer extension assays or microarrays comprising probes for specific alleles are useful formats for determining drug response. A correlation between drug responses and specific alleles' or combinations of alleles (haplotypes) of the uPA, SNCG, IDE, LIPA, TNFRSF6 and KNSL1 genes and other genes that associate with neurodegenerative disease can be shown, for example, by clinical studies wherein the response, either positive or negative, to specific drugs of subjects having different allelic variants of polymorphic regions of the uPA, SNCG, IDE, LIPA, TNFRSF6 and/or KNSL1 genes alone or in combination with allelic variants of other genes are compared. Thus, provided herein are methods for predicting a response of a subject to an agent used to treat a neurodegenerative disease or disorder which include a step of detecting in nucleic acid obtained from the subject the presence or absence of one or more polymorphisms, individually and/or in combinations, wherein the presence of the one or more polymorphisms, individually and/or in combination, is indicative of an increased or decreased likelihood that the treatment will be effective.

Such studies can also be performed using animal models, such as mice having various alleles and in which, e.g., the endogenous uPA, SNCG, IDE, LIPA, TNFRSF6 or KNSL1 genes have been inactivated such as by a knock-out mutation. Test drugs are then administered to the mice having different alleles and the response of the different mice to a specific compound is compared. Accordingly, assays, microarrays and kits are provided for determining the drug which will be best suited for treating a specific disease or condition in a subject based on the individual's genotype. For example, it will be possible to select drugs which will be devoid of toxicity, or have the lowest level of toxicity possible for treating a subject having a disease or condition, e.g., neurodegenerative disease or Alzheimer's disease.

Therapeutic agents that can be genetically profiled include, but are not limited to, ALCAR, Alpha-tocopherol (Vitamin E), Ampalex, AN-1792 (AIP-001), Cerebrolysin, Daposone, Donepezil (Aricept), ENA-713 (Exelon), Estrogen replacement therapy, Galanthamine (Reminyl), Ginkgo Biloba extract, Huperzine A, Ibuprofen, Lipitor, Naproxen, Nefiracetam, Neotrofin, Memantine, Phenserine, Rofecoxib, Selegiline (Eldepryl), Tacrine (Cognex), Xanomeline (skin patch), Resperidone (Risperidol™), Neuroleptics, Benzodiazepenes, Valproate, Serotonin reuptake inhibitors (SRIs), Beta and Gamma Secretase Inhibitors, CX-516 (Ampalex), Statins and AF-102B (Evoxac).

Other therapeutic agents include those that are neuroprotective. Drugs with anti-oxidative properties, e.g., flupirtine, N-acetylcysteine, idebenone, melatonin, and also novel dopamine agonists (ropinirole and pramipexole) have been shown to protect neuronal cells from apoptosis and thus have been suggested for treating neurodegenerative disorders like AD or PD. Also, free radical scavengers, calcium channel blockers and modulators of certain signal transduction pathways that might protect neurons from downstream effects of the accumulation of A-Beta intracellularly and/or extracellularly. Also, other agents like non-steroidal anti-inflammatory drugs (NSAIDs) partly inhibit cyclooxygenase (COX) expression, as well as having a positive influence on the clinical expression of AD. Distinct cytokines, growth factors and related drug candidates, e.g., nerve growth factor (NGF), or members of the transforming growth factor-beta (TGF-beta) superfamily, like growth and differentiation factor 5 (GDF-5), are shown to protect tyrosine hydroxylase or dopaminergic neurones from apoptosis. CRIB (cellular replacement by immunoisolatory biocapsule) is a gene therapeutical approach for human NGF secretion, which has been shown to protect cholinergic neurons from cell death when implanted in the brain ((2000) Expert Opin Investig Drugs 9(4):747-64).

As set forth above, the prognostic methods described herein may also be used to determine whether a person will respond to a particular drug. This is useful, among other things, for matching particular drug treatments to particular patient populations to thereby exclude patients for whom a particular drug treatment may be less efficacious.

Provided herein is a computer assisted method of identifying a proposed treatment-for neurodegenerative diseases (in a human subject). The method involves the steps of (a) storing a database of biological data for a plurality of patients, the biological data that is being stored include for each of said plurality of patients (i) a treatment type, (ii) the presence or absence of an allelic variant of one or more polymorphic regions of one or more genes selected from the group consisting of uPA, SNCG, IDE, KNSL1, LIPA and TNFRSF6 associated with a neurodegenerative disease (e.g., Alzheimer's Disease), and (iii) at least one disease progression measure for the neurodegenerative disease from which treatment efficacy may be determined; and then (b) querying the database to determine the dependence on said genetic polymorphism of the effectiveness of a treatment type in treating the neurodegenerative disease, to thereby identify a proposed treatment as an effective treatment for a patient carrying a particular polymorphism for the neurodegenerative disease, such as Alzheimer's Disease. In a particular embodiment, at least one of the polymorphic regions, or complements thereof, is selected from the group consisting of:
uPA nucleotide positions 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 651 9, 6909, 7235, 7848, 7908 of SEQ ID NO:559 or 560; and uPA nucleotide positions of SEQ ID NO:563 consisting of 79, 93, 256, 385 and 714 of SEQ ID NO:563;
SNCG nucleotide positions 560, 590, 61 7, 645, 915, 987, 1723, 1943, 1950, 3151, 3178, 3189, 3284, 3779, 4156, 4276, 4311, 4552, 4976, 4995, 5019, 5025, 5112, 5136, 5517, 5421, 5648, 2533, 3371, 4627, 4727, 4813 and 5200 of SEQ ID NO:73;
IDE nucleotide positions 2456, 3279, 3407, 42943, 62498, 69586,107395,112114, 116662,17095, 17242,33590, 38903, 43391, 45017, 68906, 68973, 73772, 74084, 83024, 83104, 89301, 105060, 108489, 111914, 113142, 113591, 114683, 117803 and 124565 of SEQ ID NO:187; the complement of IDE nucleotide positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, 129444, 6078, 7106, 11758, 18267, 19581, 30078, 54862, 73841, 83448, 80304, 98276, 117802 and 129124 of SEQ ID NO:484;
KNSL1 nucleotide positions 300, 1152, 14235, 15104, 20815, 35719, 36738-36739, 41015, 42125, 45083, 45887, 56706, 56887, 58524, 62661 and 63802 of SEQ ID NO:348; KNSL1 nucleotide positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, 193706, 132370, 136968, 139284, 159167, 159403, 178748, 180149 and 180153 of SEQ ID NO:484;
LIPA nucleotide positions 1197, 1307 to 1309, 1841, 1852, 2075, 6063, 6173, 6194, 7820, 25283, 28453 to 28465, 28543, 28746, 29904, 37861, 39834, 40018, 7219, 8242, 10114, 10606, 10688, 10729, 11559, 12031, 14497, 14729, 21145, 21329, 21404, 21429, 22246, 22354, 22621, 23802 and 25969 of SEQ ID NO:468; and
TNFRSF6 nucleotide positions 1530, 1550, 14525, 14714, 18982, 19069, 20412, 20552, 23199, 23416, 24890, 26359, 199, 213, 843, 2967, 3103, 5335, 5345, 6074, 9374, 9907, 9936, 10937, 11200, 11279, 11359, 11503, 11511, 11587, 11694, 11905, 12193, 12208, 12238, 18511, 18567, 20640, 21585, 22439, 25081, 26878, 27670, 1926, 2269, 18934, 19227 and 22026 of SEQ ID NO:403; and
polymorphic regions within 2 centimorgans thereof.

In one embodiment, treatment information for a patient is entered into the database (through any suitable means such as a window or text interface), genetic polymorphism information for that patient is entered into the database, and disease progression information is entered into the database. These steps are then repeated until the desired number of patients have been entered into the database. The database can then queried to determine whether a particular treatment is effective for patients carrying a particular polymorphism, not effective for patient carrying a particular polymorphism, etc. Such querying may be carried out prospectively or retrospectively on the database by any suitable means, but is generally done by statistical analysis in accordance with known techniques, as discussed further below.

Any suitable disease progression measure can be used, including but not limited to measures of motor function, measures of cognitive function, measures of dementia, etc., as well as combinations thereof. The measures are preferably scored in accordance with standard techniques for entry into the database. Measures are preferably taken at the initiation of the study, and then during the course of the study (that is, treatment of the group of patients with the experimental and control treatments), and the database preferably incorporates a plurality of these measures taken over time so that the presence, absence, or rate of disease progression in particular individuals or groups of individuals may be assessed.

An advantage of the methods produced is the relatively large number of genetic polymorphisms for Alzheimer's Disease (as set forth herein) that may be utilized in the computer-based method. Polymorphisms as set forth in the prior art, including but not limited to those described in Tables A-F herein and U.S. Pat. No. 5,508,167 to Roses et al., may also be used in combination with the polymorphisms provided herein. Thus, for example, instead of entering a single polymorphism into the database for each patient, two, three, four, five, six, seven, ten up to fifteen or more polymorphisms may be entered for each particular patient. The polymorphisms entered can either include or exclude polymorphisms of the prior art, and will be derived from one, two, three, five, seven or even ten or more polymorphisms as set forth in Tables 2, 4 and 4-B, 6 and 6-B, 8, 10, 12 and 12-B and A-F herein, and those within 2, 5, 10 or 15 centimorgans thereof, and optionally including additional polymorphisms of the prior art such as ApoE. Note that, for these purposes, entry of a polymorphism includes entry of the absence of a particular polymorphism for a particular patient. Thus the database can be queried for the effectiveness of a particular treatment in patients carrying any of a variety of polymorphisms, or combinations of polymorphisms, or who lack particular polymorphisms.

In general, the treatment type may be a control treatment or an experimental treatment, and the database preferably includes a plurality of patients having control treatments and a plurality of patients having experimental treatments. With respect to control treatments, the control treatment may be a placebo treatment or treatment with a known treatment for a neurodegenerative disease, such as Alzheimer's Disease, and preferably the database includes both a plurality of patients having control treatment with a placebo and a plurality of patients having control treatment with a known treatment for neurodegenerative diseases, such as Alzheimer's Disease.

Experimental treatments are typically drug treatments, which are compounds or active agents that are parenterally administered to the patient (i.e., orally or by injection) in a suitable pharmaceutically acceptable carrier.

Control treatments include placebo treatments (for example, injection with physiological saline solution or administration of whatever carrier vehicle is used to administer the experimental treatment, but without the active agent), as well as treatments with known agents for the treatment of a neurodegenerative disease, such as Alzheimer's Disease.

Administration of the treatments is preferably carried out in a manner so that the subject does not know whether that subject is receiving an experimental or control treatment. In addition, administration is preferably carried out in a manner so that the individual or people administering the treatment to the subject do not know whether that subject is receiving an experimental or control treatment.

Computer systems used to carry out the present invention may be implemented as hardware, software, or both hardware and software. Computer hardware and software systems that may be used to implement the methods described herein are known and available to those skilled in the art. See, e.g., U.S. Pat. No. 6,108,635 to Herren et al. and the following references cited therein: Eas, M. A.: A program for the meta-analysis of clinical trials, Computer Methods and Programs in Biomedicine, vol 53, no. 3 (July 1997); D. Klinger and M. Jaffe, An Information Technology Architecture for Pharmaceutical Research and Development, 14th Annual Symposium on Computer Applications in Medical Care, November 4-7, pp. 256-260 (Washington, D.C. 1990), M. Rosenberg, “ClinAccess: An integrated client/server approach to clinical data management and regulatory approval”, Proceedings of the 21 st Annual SAS Users Group International Conference (Cary, N.C., Mar. 10-13 1996). Querying of the database may be carried out in accordance with known techniques such as regression analysis or other types of comparisons such as with simple normal or t-tests, or with non-parametric techniques.

Accordingly, provided herein are methods of treating a subject for a neurodegenerative disease, such as Alzheimer's Disease, particularly late-onset Alzheimer's Disease, which method comprises the steps of: determining the presence or absences of a preselected polymorphism for the neurodegenerative disease in said subject; and then administering to said subject a treatment effective for treating the neurodegenerative disease, e.g., Alzheimer's Disease, in a subject that carries said polymorphism. In particular embodiments, at least one preselected polymorphism is a polymorphism selected from Tables 2, 4 and 4-B, 6 and 6-B, 8, 10, 12 and 12-B, and to which a particular treatment has been matched. A treatment is preferably identified for that polymorphism by the computer-assisted method described above.

T. KITS

Kits are provided that contain at least one container means having disposed within at least one oligonucleotide, such as a probe, primer or antisense nucleic acid molecule, that includes a sequence of nucleotides that specifically hybridizes adjacent to or at a polymorphic region of a uPA, SNCG, IDE, KNSI1, TNFRSF6 and LIPA gene or cDNA. The kits have numerous uses. For example, the kits may be used to detect the presence or absence in nucleic acid obtained from a subject of a uPA, SNCG, IDE, KNSI1, TNFRSF6 and LIPA gene polymorphism. The kits can be used to indicate whether a subject has a predisposition to, and/or protection against, developing a neurodegenerative disease (i.e. an altered level of risk for a neurodegenerative disease) and in particular Alzheimer's disease. Kits can also be used to confirm the diagnosis of a particular neurodegenerative disease. The information could also be used to optimize treatment of such individuals, as a particular genotype may be associated with a positive drug response.

Further provided are kits containing at least one container means having disposed within two or more oligonucleotides, such as primers, probes or antisense nucleic acid molecules, each of which contains a sequence of nucleotides that specifically hybridizes adjacent to-or at a polymorphic region of a uPA, SNCG, IDE, KNSI1, TNFRSF6 and LIPA gene or cDNA that includes a polymorphism that is associated, individually or in combination with other polymorphism(s), with a neurodegenerative disease or disorder, and at least two of the oligonucleotides in the kits hybridize adjacent to or at different polymorphic regions. The kits can also contain at least one container means having disposed therein at least one oligonucleotide containing a sequence that specifically hybridizes adjacent to or at a polymorphic region of another gene associated with the disease. For example, in the case of a neurodegenerative disease, e.g., Alzheimer's disease, the other gene can be, but is not limited to, APOE4.

In further particular embodiments of the kits containing at least one container means having disposed therein two or more oligonucleotides, each of which contains a sequence of nucleotides that specifically hybridizes adjacent to or at a polymorphic region of a uPA, SNCG, IDE, KNSI1, TNFRSF6 and LIPA gene wherein the polymorphic region includes a polymorphism associated individually and/or in combination with other polymorphism(s) with a neurodegenerative disease or disorder, the neurodegenerative disease or disorder is Alzheimer's disease. In particular embodiments, the disease is Alzheimer's disease with an onset age greater than or equal to about 50 years, or greater than or equal to about 60 years, or greater than or equal to about 65 years. In yet further embodiments, the association between the polymorphism, individually and/or in combination with other polymorphism(s), and Alzheimer's disease yields a positive result in a family-based test for association. In particular embodiments, the positive result is a P value less than or equal to 0.05 or the positive result is a P value less than 0.05. In further embodiments, the P value is a value obtained after correction in which the probability value required to give significance is divided by the number of tests conducted. In still further embodiments, the association between the polymorphism, individually and/or in combination with other polymorphism(s), and Alzheimer's disease yields a result in a family-based test for association that is indicative of linkage disequilibrium between the one or more polymorphisms and an allele associated with Alzheimer's disease.

Further provided are kits containing at least one container means having disposed therein two or more oligonucleotides, each of which contains a sequence of nucleotides that specifically hybridizes adjacent to or at a polymorphic region of a uPA, SNCG, IDE, KNSI1, TNFRSF6 and LIPA gene wherein the polymorphic region contains a polymorphism associated individually and/or in combination with other polymorphism(s) with a disease or disorder as follows: thrombosis, thrombolytic diseases, stroke, atherosclerosis, coronary artery disease, cardiovascular disease, cardiac disorders, myocardial infarction, cardiomyopathies, proliferative diseases, cancer, tumor angiogenesis, tumor metastasis, arthritis, rheumatic diseases or inflammatory diseases, including inflammatory joint diseases.

In another embodiment, the kits comprise at least one container means having disposed therein at least one probe or primer which is capable of hybridizing adjacent to or at a polymorphic region of uPA, SNCG, IDE, LIPA, TNFRSF6 or KNSL1 and thereby identifying whether the gene contains an allelic variant which is associated with increased susceptibility to or protection against developing a neurodegenerative disease or the presence of a neurodegenerative disease. The kits can also comprise at least one container means having disposed therein at least one probe or primer which specifically hybridizes adjacent to or at a polymorphic region of another gene associated with neurodegenerative disease.

In another embodiment, the kits can further comprise instructions for use in carrying out assays and interpreting results concerning assessing an altered level of risk for a neurodegenerative disease. Kits can also comprise other containers comprising one or more of the following: DNA amplification reagents, DNA polymerase, restriction enzymes, buffers, wash reagents and reagents capable of detecting the presence of bound nucleic acid probes. Examples of detection reagents include, but are not limited to, radiolabelled probes, enzymatic labeled probes (horseradish peroxidase, alkaline phosphatase) and affinity labeled probes (biotin, avidin, or streptavidin).

In detail, a compartmentalized kit includes any kit in which reagents are contained in separate containers. Such containers include small glass containers, plastic containers or strips of plastic,or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain the reagents used to detect the hybridized probe, bound antibody, amplified product, or the like.

Types of detection reagents include labeled secondary probes, or in the alternative, if the primary probe is labeled, the enzymatic, or antibody binding reagents which are capable of reacting with the labeled probe. One skilled in the art will readily recognize that the disclosed probes and amplification primers can readily be incorporated into one of the established kit formats which are well known in the art.

Kits for amplifying a region of uPA, SNCG, IDE, LIPA, TNFRSF6 and/or KNSL1 genes, or other genes associated with neurodegenerative disorders comprise two primers which flank a polymorphic region of the gene of interest. For other assays, primers or probes hybridize to a polymorphic region or 5′ or 3′ to a polymorphic region depending on which strand of the target nucleic acid is used. For the SNCG gene the polymorphic regions include, but are not limited to, positions corresponding to positions 560, 590, 617, 645, 915, 987, 1723, 1943, 1950, 3151, 3178, 3189, 3284, 3779, 4156, 4276, 4311, 4552, 4976, 4995, 5019, 5025, 5112, 5136, 5517, 2533, 3371, 4627, 4727, 4813 and/or 5200 of SEQ ID NO:73 or the complement thereof. For the IDE gene the polymorphic regions include, but are not limited to, positions corresponding to positions 2456, 3279, 3407, 42943, 62498, 69586, 107395, 112114, and/or 116662 of SEQ ID NO: 187, or the complement thereof, or corresponding to SEQ ID NO:484 positions 820, 7066, 11758, 21270, 22225, 29294, 33452, 33708, 36982, 54862, 77786, 80594, 84792, 84997, 86682, 86857, 88511, 90437, 90593, 91650, 91870, 91878, 92011, 93618, 94344, 94714, 95671, 96324, 97302, 97370, 98253, 98276, 98385, 98646, 98814, 99597, 100378, 101029, 101265, 102465, 103289, 103967, 105793, 106076, 106453, 106600, 106995, 107851, 108434, 109096, 109399, 109483, 110870, 111189, 111972, 112627, 112629, 112631, 113407, 114444, 114482, 115473, 116681, 117226, 117600, 117802, 118223, 120011, 122260, 123165, 123424, 124352, 124501, 124692, 125113, 125159, 126568, 127166, 127598, 127600, 127609, 127614, 127623, 127662, 128053, 128261, 128289, 128291, 128393, and 129444, or the complement thereof. For the KNSL1 gene the polymorphic regions include, but are not limited to, positions corresponding to positions 300, 1152, 14235, 15104, 20815, 35719, 36738, 36739, 41015, 42125, 45083, 45887, 56706, 56887, 58524, 62661 and/or 63802 of SEQ ID NO:348, or to SEQ ID NO:484 positions 130876, 131378, 131616, 131620, 131688, 131998, 132004, 132370, 132697, 132968, 133355, 133806, 134030, 134291, 134661, 137087, 137142, 138396, 140665, 140736, 141173, 142056, 142777, 143025, 143729, 144484, 146181, 147051, 147322, 147707, 147842, 148080, 149026, 149044, 149389, 150003, 150384, 150454, 150686, 151343, 151961, 152119, 153791, 154328, 154513, 154639, 155049, 155114, 158040, 158895, 191284, 192272, 192698, and 193706, or the complement thereof. For the TNFRSF6 gene the polymorphic regions include, but are not limited to, positions corresponding to positions 1530, 1550, 14525, 14714, 18982, 19069, 20412, 20552, 23199, 23416, 24890, 26359, 1926, 2269, 18934, 19227 and/or 22026 of SEQ ID NO:403, or the complement thereof. For the LIPA gene the polymorphic regions include, but are not limited to, positions corresponding to positions 1197, 1307-1309, 1841, 1852, 2075, 6063, 6173, 6194, 7820, 25283, 28453-28465, 28543, 28746, 29904, 37861, 39834 and/or 40018 of SEQ ID NO:468, or the complement thereof. Those of skill in the art can synthesize primers and probes which hybridize adjacent to or at the polymorphic regions described herein and other polymorphisms in genes associated with neurodegenerative diseases.

In another embodiment a kit contains at least one container means having disposed within, at least one oligonucleotide, such as a probe, primer or antisense nucleic acid molecule, containing a sequence of nucleotides that specifically hybridizes adjacent to or at a polymorphic region of a uPA gene spanning a nucleotide position, or the complementary position thereof, corresponding to positions selected from the group consisting of nucleotide positions 9, 401, 464, 515, 748, 1229, 1356, 1752, 1942, 2127, 2543, 3029, 3169, 3799, 3947, 4808, 5287, 6532, 178, 1363, 1423, 1465, 1540, 2297, 2445, 2653, 3080, 3546, 3664, 3816, 4320, 4369, 4399, 4851, 5186, 5204, 5787, 6519, 6909, 7235, 7848, and 7908 of SEQ ID NO:559 or 560; and positions of SEQ ID NO:563 consisting of 79, 93, 256, 385 and 714; and the complementary positions thereof. In another embodiment, the kit further comprises at least one other container means having disposed within at least one oligonucleotide, e.g., probe or primer, which specifically hybridizes at or adjacent to at least one polymorphic region of another gene associated with neurodegenerative disease. In a particular embodiment, the other gene is APOE4.

Yet other kits comprise at least one reagent necessary to perform an assay. For example, the kit can comprise an enzyme, such as a nucleic acid polymerase. Alternatively the kit can comprise a buffer or any other necessary reagent.

Yet other kits comprise microarrays of probes to detect allelic variants of uPA, SNCG, IDE, LIPA, TNFRSF6 and/or KNSL1 with Alzheimer's disease. The kits further comprise instructions for their use and interpreting the results.

U. Combinations

Provided herein are combinations of reagents for a number of uses. For example, the combinations may be used to detect the presence or absence in nucleic acid obtained from a subject of a uPA, SNCG, IDE, KNSL1, TNFRSF6 and LIPA gene polymorphism. Other uses of the combinations include, but are not limited to, indicating a predisposition to, the occurrence of and/or a level of risk for a disease or disorder, such as, for example, a neurodegenerative disease, e.g., Alzheimer's disease. The combinations also can be used to confirm a diagnosis of a particular disease. The combinations can be provided as a kit, which optionally includes instructions for using the reagents for the above-noted purposes. The reagents are provided in appropriate packaging, such as containers, blister packs, linked to solid supports and any other suitable packaging. Included among the components of the combinations are those described below for the kits. Such components include, but are not limited to, oligonucleotides, primers, probes, antisense nucleic acid molecules, mixtures of primers, mixtures of probes, and reagents for use with the probes and primers. Suitable primers and probes are described throughout the disclosure herein (see section entitled Probes, Primers and Antisense Nucleic acids and other Oligonucleotides, and, see the section entitled Kits).

In a particular embodiment, the combination contains two or more, or three or more, oligonucleotides, such as, for example, primers, probes and antisense nucleic acid molecules, wherein each oligonucleotide contains a sequence of nucleotides that specifically hybridizes adjacent to or at either strand of a polymorphic region provided herein of a uPA, SNCG, IDE, KNSL1, TNFRSF6 and LIPA gene or cDNA wherein the polymorphic region contains a polymorphism associated, individually and/or in combination with other polymorphisms, with a neurodegenerative disease or disorder, and at least two of the oligonucleotides, such as primers, probes or antisense molecules, hybridize adjacent to or at different polymorphic regions.

In particular embodiments of any of the above combinations, each oligonucleotide contains at least 10, 14, 15, 16, 17, 20, 30, 40, 50, 60, 70, 80, 90 or 100 contiguous nucleotides of a uPA, SNCG, IDE, KNSL1, TNFRSF6 and LIPA gene or cDNA provided herein, such as in the Sequence Listing.

In a particular embodiment of the combinations of oligonucleotides containing a sequence of nucleotides that specifically hybridizes adjacent to or at either strand of a polymorphic region of a uPA, SNCG, IDE, KNSL1, TNFRSF6 and LIPA gene or cDNA wherein the polymorphic region contains a polymorphism associated, individually and/or in combination with other polymorphisms, with a neurodegenerative disease or disorder, the disease is Alzheimer's disease. In a further particular embodiment, the disease is Alzheimer's disease with an onset age greater than or equal to about 50 years, or greater than or equal to about 60 years, or greater than or equal to about 65 years. In yet further particular embodiments of the combinations, the association between the polymorphism, individually and/or in combination with other polymorphism(s), and Alzheimer's disease yields a positive result in a family-based test for association. In particular embodiments, the positive result is a P value less than or equal to 0.05 or the positive result is a P value less than 0.05. In yet further embodiments, the P value is a value obtained after correction in which the probability value required to give significance is divided by the number of tests conducted. In further embodiments, the association between the polymorphism, individually and/or in combination with other polymorphism(s), and Alzheimer's disease yields a result in a family-based test for association that is indicative of linkage disequilibrium between the polymorphism and an allele associated with Alzheimer's disease.

V. Computer Readable Medium

The nucleic acid sequences relating to and identifying uPA, SNCG, IDE, KNSL1, TNFRSF6 and LIPA gene polymorphisms represent a valuable information source with which to identify further sequences of similar identity and characterize individuals in terms of, for example, their identity, haplotype and other sub-groupings, such as susceptibility to treatment with particular drugs. These approaches are most easily facilitated by storing the sequence information in a computer readable medium and then using the information in standard macromolecular structure programs or to search sequence databases using state of the art searching tools such as GCG (Genetics Computer Group), BlastX, BlastP, BlastN, FASTA [Altschul et al. (1990) J. Mol. Biol. 215:403-4101. Thus, the nucleic acid sequences containing polymorphisms of a uPA, SNCG, IDE, KNSL1, TNFRSF6 and LIPA gene are particularly useful as components in databases useful for sequence identity, genome mapping, pharmacogenetics and other search analyses. Generally, the sequence information relating to the nucleic acid sequences and polymorphisms of a uPA, SNCG, IDE, KNSL1, TNFRSF6 and LIPA gene may be reduced to, converted into or stored in a tangible medium such as a computer disk, preferably in a computer readable form. For example, chromatographic scan data or peak data, photographic scan or peak data, mass spectrographic data, sequence gel (or other) data.

Provided herein is a computer readable medium having stored thereon one or more nucleic acid sequences provided herein. Nucleic acid sequences provided herein include, for example, each of the nucleic acid sequences set forth herein, e.g., the “Nucleic Acid Molecules” section of the summary.

For example, a computer readable medium is provided containing and having stored thereon a member selected from the group consisting of: a nucleic acid sequence provided herein, a nucleic acid sequence containing a nucleic acid sequence provided herein, a nucleic acid sequence containing part of a nucleic acid sequence provided herein, wherein the part includes at least one of the uPA, SNCG, IDE, KNSL1, TNFRSF6 and LIPA gene polymorphisms provided herein, a set of nucleic acid sequences wherein the set includes at least one nucleic acid sequence provided herein, a data set containing or consisting of a nucleic acid sequence provided herein or a part thereof containing at least one of the uPA, SNCG, IDE, KNSL1, TNFRSF6 and LIPA gene polymorphisms provided herein. The computer readable medium can be any composition of matter used to store information or data, including, for example, floppy disks, tapes, chips, compact disks, digital disks, video disks, punch cards and hard drives.

Provided herein is a computer readable medium having stored thereon a nucleic acid sequence containing at least 14, at least 15, at least 16, at least 17, or at least 20 consecutive bases of a uPA, SNCG, IDE, KNSL1, TNFRSF6 and LIPA gene sequence, which sequence includes at least one polymorphism at a position corresponding to a nucleotide position set-forth herein, or the complementary positions thereof.

A computer-based method is also provided for performing sequence identification, wherein the method includes the steps of providing nucleic acid sequence containing a polymorphism provided herein in a computer readable medium; and comparing said polymorphism-containing nucleic acid sequence to at least one other nucleic acid or polypeptide sequence to identify identity (homology), i.e., screen for the presence of a polymorphism. Such a method is particularly useful in pharmacogenetic studies and in genome mapping studies.

In another embodiment, there is provided a method for performing sequence identification, said method including the steps of providing a nucleic acid sequence containing at least 14, at least 15, at least 16, at least 17, or at least 20 consecutive bases of a uPA, SNCG, IDE, KNSL1, TNFRSF6 or LIPA gene sequence, which sequence includes at least one polymorphism provided herein, or the complementary positions thereof, in a computer readable medium; and comparing said nucleic acid sequence to at least one other nucleic acid sequence to identify identity. In a particular embodiment of this method, the nucleic acid sequence is one of the nucleic acid sequences provided herein.

The following examples are included for illustrative purposes only and are not intended to limit the scope of the invention. The practice of methods and development of the products provided herein employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook, Fritsch and Maniatis (1989) “Molecular Cloning A Laboratory Manual,” 2d ed. Cold Spring Harbor Laboratory Press; “DNA Cloning,” (1985) Vols I and II D. N. Glover ed.; “Oligonucleotide Synthesis,” (1984) M. J. Gaited.; Mullis etal. U.S. Pat. No. 4,683,195; “Nucleic Acid Hybridization,” (1984) B. D. Hames & S. J. Higgins eds.; “Transcription and Translation,” (1984) B. D. Hames & S. J. Higgins eds.; “Culture of Animal Cells,” (1987) R. I. Freshney, Alan R. Liss, Inc.; “Immobilized Cells and Enzymes,” (1986) IRL Press; B. Perbal (1984) “A Practical Guide To Molecular Cloning”; “The treatise, Methods In Enzymology,” Academic Press, Inc. (New York); “Gene Transfer Vectors For Mammalian Cells,” (1987) J. H. Miller and M. P. Calos eds. (Cold Spring Harbor Laboratory); “Methods In Enzymology,” Vols. 154 and 155 Wu et al. eds.; “Immunochemical Methods In Cell and Molecular Biology,” (1987) Mayer and Walker, eds. Academic Press (London); “Handbook of Experimental Immunology,” (1986) Vols l-IV D. M. Weir and C. C. Blackwell, eds.; “Manipulating the Mouse Embryo,” (1986) Cold Spring Harbor Laboratory Press (Cold Spring Harbor, N.Y.).

The following examples are included for illustrative purposes only and are not intended to limit the scope of the invention.

### EXAMPLE 1

Linkage to Chromosome 10

Microsatellite markers on human chromosome 10 were analyzed for genetic linkage to AD. The analysis was conducted by genotyping genomic DNA samples from AD family members with respect to seven microsatellite markers and performing parametric and non-parametric analyses of genotyping data. Genetic linkage analysis has identified highly polymorphic markers with significant linkage to Alzheimer's disease on human chromosome 10 (10q23-24).

The genomic DNA utilized in the linkage analyses was from the full National Institute of Mental Health (NIMH) Genetics Initiative sample of AD family DNA (Blacker et al. (1997) Neurology 48:1 39-147). Through the NIMH Genetics Initiative, a national resource of clinical data and biomaterials (DNA samples) collected from individuals with AD has been established. AD pedigrees have been ascertained by three extramural sites (Massachusetts General Hospital/Harvard Medical School, University of Alabama and Johns Hopkins University) and data collection has been coordinated among the three sites by using a common protocol that includes uniform assessments and medical, neurologic and psychiatric histories.

In generating the NIMH sample, subjects were collected following a standardized protocol applying NINCDS/ADRDA (National Institute of Neurological and Communicative Disorders and Stroke/Alzheimer's Disease and Related Disorders Association) criteria for the diagnosis of AD (Blacker et al. (1997) Neurology 48:139; McKhann et al. (1984) Neurology 34:939), The diagnostic process in the NIMH AD Genetics Initiative includes a systematic and comprehensive examination of all available information from autopsy records, family history, medical records, and patient and/or informant interviews. Definite AD according to age-adjusted Khachaturian criteria is diagnosed on autopsy. Operational criteria for the clinical diagnosis of probable or possible AD following NINCDS-ADRDA Work Group guidelines have been developed and are implemented by the three sites. Case summaries for all subjects with a clinical diagnosis of probable or possible AD are reviewed by the site principal investigators and a procedure has been implemented to establish a consensus diagnosis. Subjects are followed longitudinally to track changes in diagnoses and to compare diagnoses by autopsy.

Only families in which all sample affected had onset ages ≧50 years were included (n=435 families; n=1426 subjects, mean age of onset=72.5±7.7 years, range 50-97 years). The original sample included a total of 1500 subjects from 449 families with two or more affected subjects per family. Families in which any sampled individual had an onset age less than 50 years (n=14 families and 74 individuals) were excluded, yielding 1426 individuals from 435 families for this analysis, including 993 affected individuals, 429 unaffected, and 4 with phenotype unknown. Over the 10 years that the NIMH sample has been followed, a clinical diagnosis of AD has been confirmed at autopsy in 94% of the cases. All DNA samples are stored in a centralized cell repository at Rutgers University, New Brunswick, N.J.

The results of the parametric two-point analyses of seven microsatellite markers on chromosome 10 in 435 AD families using a dominant model revealed significant evidence of linkage of AD to chromosome 10 around marker D10S583 (Zₘₐₓ3.3) in a full sample and around marker D10S1671 in the late-onset sample (Zₘₐₓ=3.4). The results of the parametric two-point analyses using a recessive model were similar, with a maximum LOD score of 3.8 for marker D10S1671 in the late-onset sample.

The results of two-point non-parametric linkage analyses also revealed linkage of AD on chromosome 10 q with the highest linkage scores (Zₗᵣ=Z scores for the likelihood ratio) provided by markers D10S583, D10S1710 and D10S1671 (Z,r scores of 2.8, 2.8 and 3.8, respectively, for the late-onset dataset). Multipoint non-parametric analyses generated maximum Z,r scores of 1.9 (p=0.029, full sample), 2.1 (p=0.02, late onset) and 2.15 (p=0.016, APOE E4-negative) at marker D10S1710, which is located between the two markers (i.e., D10S583 and D10S1671) with the greatest linkage signals in the two-point analyses.

Marker D1QS583 was analyzed for association with AD using the Family-Based Association Test computer program (FBAT) (Rabinowitz and Laird (2000) Hum. Hered. 50:211-223) to determine if it is within linkage disequilibrium range of an underlying disease gene. The analyses were based on estimated empirical variances (to account for the presence of linkage) (Lake et al. (2000) Am. J. Hum. Genet. 67:1515-1525) as implemented in FBAT (Version 1.0, 1999). Although the multiallelic test on al 11 alleles for marker D10S583 was not significant (p=0.15), the diallelic test revealed significant association of the 211-bp allele with an allele that is protective against AD (nominal p=0.004, Bonferroni corrected p=0.04).

The results of the linkage and association analyses of markers on human chromosome 10 indicate the presence of multiple, e.g., two, loci underlying AD on chromosome 10; at least one DNA segment that causes AD or confers increased susceptibility to AD, as well at least one DNA segment that is protective against AD. A protective allele generally has a counterpart disease risk allele.

Sequencing of regions of chromosome 10 was carried out to identify mutations that may be responsible for the observed linkage peaks. Candidate genes were selected by proximity to markers D10S583 and D10S1671 on chromosome 10. Other factors considered in the selection of candidate genes included physiological relevance to the disease and expression in brain. Based on these criteria, six candidate genes were chosen for sequencing: uPA, SNCG, IDE, KNSL1, TNFRSF6 and LIPA. High throughput genomic DNA sequencing of candidate genes in DNA samples obtained from the NIMH led to the discovery of novel polymorphisms in these genes and surrounding regions in chromosome 10.

### EXAMPLE 2

Sequencing of SNCG Candidate Gene

The nucleotide sequence of the SNCG gene in two sets of human genomic DNA samples was determined. The “SNCG” set was derived from nine members of three families that showed Alzheimer's disease (AD) linkage to D10S583 and association with a silent mutation in SNCG exon 3 (NCBI reference SNP number rs760113; P=0.02 in SDT). The “D10-Top10” set was derived from 10 members of three families with the highest scores for AD linkage to genetic marker D1QS583. Each set contained individuals both affected and unaffected with AD. Seven of the SNCG and all of the D10-Top10 samples were sequenced in this study.

The 5 SNCG exons, 4 introns, and approximately 500 bp each of 5′ and 3′ flanking sequence were amplified by PCR from each sample in 5 overlapping amplicons. The nucleotide sequence on both strands was determined using nested sequencing primers spaced at approximately 250-300-bp intervals. The PCR and sequencing primers (shown in Table 1) were designed using OLIGO6.0 software (Molecular Biology Insights, Inc., Cascade, Colo.). The nucleotide sequence template for primer design consisted of a human SNCG genomic sequence (GenBank accession AF044311) plus an additional 909 nucleotides of 5′ flanking sequence obtained from a BLAST search of the NCBI human EST database. The complete primer design template sequence is set forth in SEQ ID NO:483. After sequencing, it was determined that the products obtained from the samples were more similar to sequence AF037207 (SEQ ID NO:72) than AF04431 1. The above genomic sequences (GenBank accession Nos. AF044311 and AF037207) and two cDNA sequences corresponding to GenBank accession Nos. AF010126 and AF017256, were used to identify polymorphic regions (e.g., SNPs, and the like) in the SNCG gene.

SNCG PCR1-PCR5 products were amplified by polymerase chain reaction (PCR) from the SNCG set (SNCG-1, -2, and -5 through -9) and 10 samples from the D10-Top10 set (D10-1 through D10-8a). A mixture of 20 ul HotStarTaq Master Mix (QIAGEN, Valencia, Calif.), 12 ul DNA (2 ng/ul), and 8 ul primer mix (100 ng/ul each of the appropriate forward and reverse primers) was subjected to the following thermocycle: 15 min 95° C., 35×(15 sec 94° C., 45 sec TA, 2 min 72° C.), 7 min 72° C., where T_{A}=62° C. (PCRs 1, 4 and 5) or 65° C. (PCRs 2-3). The PCR products were purified with the QIAquick PCR Purification Kit (QIAGEN, Valencia, Calif.) according to the manufacturer's protocol. Product concentrations were estimated using PicoGreen reagent (Molecular Probes, Inc., Eugene, Oreg.) according to the manufacturer's protocol. PCR1 products were diluted to 2.5 ng/ul; PCR2-5 products were diluted to 5.0 ng/ul. Sequencing reactions were performed with BigDye version 2 (Applied Biosystems, Foster City, Calif.) as follows: a mixture of 4 ul BigDye Reagent, 4 ul PCR product, and 2 ul sequencing primer (1.6. μM) was subjected to 30 cycles of (10 sec 96° C., 5 sec 50° C., 4 min 60° C.). Sequence products were detected and analyzed on an ABI 3700 automated sequencer (Applied Biosystems, Foster City, Calif.) according to the manufacturer's protocol. Sequence data were analyzed using Sequencher software (GeneCodes Corp., Ann Arbor, Mich.).

Primers used for PCR and sequencing are shown in Table 1.

TABLE 1

| | | | |
|---|---|---|---|
| | | SEQ | |
| | | ID | |
| NAME | SEQUENCE (5′ → 3′) | NO: | PURPOSE |

| | | | |
|---|---|---|---|
| SNCG01-1 | GGTTGGCTGCTCTCACCTAACAG | 1 | PCR primer |
| SNCG01-2 | GGGAGTAAGGTCTGGCGAAGGTA | 2 | PCR primer |
| SNCG01-3 | TAGGAAGCTGGGGACAAT | 3 | Sequencing |
| | | | primer |
| SNCG01-4 | TATGTGGCCCTGACCCCTAC | 4 | Sequencing |
| | | | primer |
| SNCG01-5 | CCAACCTGTCCCTCATTC | 5 | Sequencing |
| | | | primer |
| SNCG01-6 | CTGGTCTCTGCCTTCCTA | 6 | Sequencing |
| | | | primer |
| SNCG01-7 | CCGATGCCTCCTATTGAC | 7 | Sequencing |
| | | | primer |
| SNCG01-8 | TGGATGTCTTCAAGAAGG | 8 | Sequencing |
| | | | primer |
| SNCG01-9 | TCTGGCGAAGGTAACTGG | 9 | Sequencing |
| | | | primer |
| SNCG02-1 | CATCAATATTTCATCGGCGTCAAT | 10 | PCR primer |
| SNCG02-2 | GGGGTGGTTGTGGTGATTCC | 11 | PGR primer |
| SNCG02-3 | CATCGGCGTCAATAGGAG | 12 | Sequencing |
| | | | primer |
| SNCG02-4 | GGCGTCAATAGGAGGCATCG | 13 | Sequencing |
| | | | primer |
| SNCG02-5 | TTCGCCAGACCTTACTCC | 14 | Sequencing |
| | | | primer |
| SNCG02-6 | GGTGAGTGCCCAGTTACCTT | 15 | Sequencing |
| | | | primer |
| SNCG02-7 | CCGAGGAGGCCAAAGACA | 16 | Sequencing |
| | | | primer |
| SNCG02-8 | GGTAGCCAGCCCTGTCTGC | 17 | Sequencing |
| | | | primer |
| SNCG02-9 | TCCCTCCCCAGAGAGAAG | 18 | Sequencing |
| | | | primer |
| SNCG02-10 | ACACGGGAGCGGCTACAG | 19 | Sequencing |
| | | | primer |
| SNCG02-11 | GGCCAGAGCTGAGTCATT | 20 | Sequencing |
| | | | primer |
| SNCG02-12 | CCTCAGAAGCAGCAACAG | 21 | Sequencing |
| | | | primer |
| SNCG02-13 | CCTGCTCTCTCTTGTCCC | 22 | Sequencing |
| | | | primer |
| SNCG02-14 | CAAACACAACAGAACAGC | 23 | Sequencing |
| | | | primer |
| SNCG02-15 | GAGCTGTCAGCCCAGACCTC | 24 | Sequencing |
| | | | primer |
| SNCG02-16 | GGGCTCCTGCATCCTAGT | 25 | Sequencing |
| | | | primer |
| SNCG02-17 | CTCCAGGGCACCTCTGAT | 26 | Sequencing |
| | | | primer |
| SNCG02-18 | CCCATAGTGGCCGAGAAG | 27 | Sequencing |
| | | | primer |
| SNCG02-19 | GACCAAGGAGCAGGCCAAC | 28 | Sequencing |
| | | | primer |
| SNCG02-20 | GAGAGGACTGGGCAGGTT | 29 | Sequencing |
| | | | primer |
| SNCG02-21 | AGAGGACTGGGCAGGTCTGA | 30 | Sequencing |
| | | | primer |
| SNCG03-1 | TGTCCTCCCATAGTGGCCGAGAA | 31 | PCR primer |
| SNCG03-2 | CAGCCCAGGGAAGCCGACAC | 32 | PCR primer |
| SNCG03-3 | AGGCGGCATCACTCCACT | 33 | Sequencing |
| | | | primer |
| SNCG03-4 | GCCCATCTCATAGACAAGGA | 34 | Sequencing |
| | | | primer |
| SNCG03-5 | TTCCAGACCCGAATGCAG | 35 | Sequencing |
| | | | primer |
| SNCG03-6 | TTGTCTATGAGATGGGCTCC | 36 | Sequencing |
| | | | primer |
| SNCG03-7 | GACGGCTCTCAGCACTTA | 37 | Sequencing |
| | | | primer |
| SNCG03-8 | GCTTTCTTACCACTAGGG | 38 | Sequencing |
| | | | primer |
| SNCG03-9 | CGTGTGCGGCCCAATAGC | 39 | Sequencing |
| | | | primer |
| SNCG03-10 | ACAATGCGTAAAGCAGAG | 40 | Sequencing |
| | | | primer |
| SNCG03-11 | AGAGCCTCAGACTCCACC | 41 | Sequencing |
| | | | primer |
| SNCG03-12 | GGTGGCCTGGGCTAAGAT | 42 | Sequencing |
| | | | primer |
| SNCG03-13 | CAGGGAAGCCGACACTCT | 43 | Sequencing |
| | | | primer |
| SNCG04-1 | TAAGTGGTGGCCTGGGCTAAGAT | 44 | PCR primer |
| SNCG04-2 | CCTCGTGGCCTTGTTGTACTTG | 45 | PCR primer |
| SNCG04-3 | CTGGGCCTCCGTGTTCTC | 46 | Sequencing |
| | | | primer |
| SNCG04-4 | CCGCTCCCTCTCCTAGTTC | 47 | Sequencing |
| | | | primer |
| SNCG04-5 | TGGCTCCAAGGGCTCACT | 48 | Sequencing |
| | | | primer |
| SNCG04-6 | CTGGTGACACCCCAAAAC | 49 | Sequencing |
| | | | primer |
| SNCG04-7 | AGTGAGCCCTTGGAGCCAC | 50 | Sequencing |
| | | | primer |
| SNCG04-8 | GTGATTGCCCTGCACTCT | 51 | Sequencing |
| | | | primer |
| SNCG04-9 | TGCTGTGCTGGCGGGAGT | 52 | Sequencing |
| | | | primer |
| SNCG04-10 | CCAGAGTGCAGGGCAATC | 53 | Sequencing |
| | | | primer |
| SNCG04-11 | GCAGGTGCTGAGGCCAGAGT | 54 | Sequencing |
| | | | primer |
| SNCG04-12 | GCTCAGGGCCTTCCAACT | 55 | Sequencing |
| | | | primer |
| SNCG04-13 | TGGGGTTCACATAGGGACTC | 56 | Sequencing |
| | | | primer |
| SNCG04-14 | TGGCCTCAGAGTCCCTAT | 57 | Sequencing |
| | | | primer |
| SNCG04-15 | ACTCTAACCCCCACTCCAC | 58 | Sequencing |
| | | | primer |
| SNCG04-16 | GGCGTGCAATTCAGACAA | 59 | Sequencing |
| | | | primer |
| SNCG05-1 | CTCCCTGGCCTCAGAGTCCCTAT | 60 | PCR primer |
| SNCG05-2 | GCCCTGTTCCTCTTGCGACACT | 61 | PCR primer |
| SNCG05-3 | CGGGTATTGTCTGAATTG | 62 | Sequencing |
| | | | primer |
| SNCG05-4 | AAGTACAACAAGGGCACGAG | 63 | Sequencing |
| | | | primer |
| SNCG05-5 | GGGTCCCTACCAAGGAAC | 64 | Sequencing |
| | | | primer |
| SNCG05-6 | TCCAAAGAGAAAGAGGAA | 65 | Sequencing |
| | | | primer |
| SNCG05-7 | GGAAGTGGCAGAGGAGGTAG | 66 | Sequencing |
| | | | primer |
| SNCG05-8 | TGTAGCCCTCTAGTCTCC | 67 | Sequencing |
| | | | primer |
| SNCG05-9 | AGACTAGAGGGCTACAGG | 68 | Sequencing |
| | | | primer |
| SNCG05-10 | AGGAGTGGGCTCAAGTTT | 69 | Sequencing |
| | | | primer |
| SNCG05-11 | GAACGGGTTCAGCCTGTT | 70 | Sequencing |
| | | | primer |
| SNCG05-12 | GGAGCCCAGAACTCACAT | 71 | Sequencing |
| | | | primer |

SNCG PCR fragment SNCG-01 contains exon 1 and flanking sequence which includes the presumed promoter-containing region. SNCG PCR fragment SNCG-02 overlaps SNCG-01 and contains exons 1 through 3 and flanking sequence. SNCG PCR fragment SNCG-03 overlaps SNCG-02 and contains parts of exon 3 and intron 3. SNCG PCR fragment SNCG-04 overlaps SNCG-03 and contains additional intron 3 sequence. SNCG-05 overlaps SNCG-04 and contains the remainder of intron 3 plus exon 5 and flanking sequence. SNCG PCR fragment #1 was amplified from genomic DNA with primers SNCGO1-1 and SNCGO1-2 and sequenced with primers SNCGO1 -3 through -9 (see Table 1). SNCG PCR fragment #2 was amplified from genomic DNA with primers SNCGO2-1 and SNCGO2-2 and sequenced with primers SNCGO2-3 through −21. SNCG PCR fragment #3 was amplified from genomic DNA with primers SNCGO3-1 and SNCGO3-2 and sequenced with primers SNCGO3-3 through −13. SNCG PCR fragment #4 was amplified from genomic DNA with primers SNCGO4-1 and SNCGO4-2 and sequenced with primers SNCG04-3 through −17. SNCG PCR fragment #5 was amplified from genomic DNA with primers SNCGO5-1 and SNCGO5-2 and sequenced with primers SNCGO5-3 through −12.

Polymorphic regions were discovered as samples contained nucleotides that differed from the reference nucleotide sequence corresponding to GenBank accession No. AF037207 plus an additional 175 nucleotides of 3′ flanking sequence corresponding to the reverse complement of nucleotides 235901-236075 of GenBank accession No. AC025039.4 (SEQ ID NO:72) at specific nucleotide positions. Table 2 shows the polymorphic regions (e.g., SNPs, and the like) that were found along with the type of nucleotide polymorphic change detected relative to the SNCG reference sequence set forth as SEQ ID NO:72. Table 2 also includes five putative SNPs (1e.CDS+9, 1e.CDS+37, 5e.3′UTR+80, 5e.3′UTR+99, and 5e.3′UTR+129) that were identified as differences between sequences in GenBank, but not yet confirmed experimentally.

TABLE 2
(SNCG Polymorphic Regions)

| POLYMORPHISM | POSITION IN SEQ ID | PUBLIC | NUCLEOTIDE |
|---|---|---|---|
| NAME | NO: 72 | DATABASE? | CHANGE DETECTED |
| 1e.5′UTR-19 | 590 | YES | A → C |
| 1i.D + 186 | 915 | NO | T → G |
| 1i.D + 258 | 987 | NO | C → A |
| 2i.A-189 | 1723 | YES | A → G |
| 3e.CDS + 195 | 1943 | YES | G → C |
| 3i.D + 495 | 2533 | NO | T → G |
| 3i.D + 1112 | 3151 | NO | A → G |
| 3i.D + 1139 | 3178 | NO | T → C |
| 3i.D + 1150 | 3189 | NO | T → C |
| 3i.D + 1245 | 3284 | NO | G → A |
| 3i.A-1143 | 3371 | NO | A → C |
| 3i.A-736 | 3779 | NO | Deletion of T |
| 3i.A-359 | 4156 | NO | Insert G |
| 3i.A-239 | 4276 | NO | T → A |
| 3i.A-204 | 4311 | NO | C → T |
| 4i.D + 41 | 4627 | NO | T → G |
| 4i.D + 141 | 4727 | NO | A → G |
| 4i.A-63 | 4813 | NO | A → C |
| 5e.3′UTR + 123 | 5019 | YES | C → T |
| 5e.3′UTR + 240 | 5136 | NO | T → A |
| ds + 31 | 5200 | NO | G → C |
| ds + 348 | 5517 | NO | T → C |

As shown in Table 2, the results indicated the detection of 18 polymorphic regions corresponding to SNPs, and single-nucleotide insertions and deletions, in the two sample sets, as well as five polymorphic regions identified by differences in reported GenBank sequences. Twelve of these polymorphic regions corresponding to SEQ ID NO:73, positions: 915, 987, 2533, 3151, 3178, 3189, 3284, 3371, 3779, 4156, 4276, 4311, 4627, 4727, 4813, 5136, 5200 and 5517 have yet to be described in public databases or literature.

5′UTR Sequence

The SNP corresponding to 1e.5′UTR-19 is located in the 5′ untranslated sequence and may affect, among other processes, translation initiation and RNA stability.

Intervening Sequences

The SNPs corresponding to 1i.D+186 and 1i.D+258 of Table 2 are positioned in an intron between exon 1 and exon 2. The SNP corresponding to 2i.A-1 89 of Table 2 is positioned in an intron between exons 2 and 3. The SNPs corresponding to 3i.D+1112, 3i.D+1139, 3i.D+1150, 3i.D+1245, 3i.A-736, 3i.A-359, 3i.A-239 and 3i.A-204 of Table 2 are positioned in an intron between exons 3 and 4 of the SNCG gene. These intron-region SNPs may affect splicing (see, e.g., Dredge et al. (2001) Nature Reviews 2:43-50; D'Souza et al. (1999) PNAS, U.S.A. 96:5598-5603; Grover etal. (1999) J. Biol. Chem. 274:15134-15143), and the like.

Coding Sequence

The SNPs located at positions corresponding to 1e.5′UTR-19, 1e.CDS+9, 1e.CDS+37 are positioned in the first exon, where 1e.CDS+37 results in a change in amino acid residue 13 from E to K. The SNPs located at positions corresponding to 3e.CDS+195 and 3e.CDS+202 are positioned in the third exon of the coding region, where 3e.CDS+202 results a change in amino acid residue 68 from E to K. The SNP located at 4e.CDS+329 is in the fourth exon.

3′ UTR Sequence

The SNPs corresponding to 5e.3′UTR+80, 5e.3′UTR+99, 5e.3′UTR+123, 5e.3′UTR+129 and 5e.3′UTR+240 are located in the 3′ untranslated sequence and may affect, among other processes, stability, RNA processing, and polyadenylation of the SNCG transcript.

3′ Flanking Sequence

The SNP corresponding to ds+348 is located downstream from the SNCG gene, in a region that may contain cis-acting elements capable of modulating SNCG gene expression.

Other known SNPs in the SNCG gene contemplated for use in the various diagnostic and screening methods, as well as kits and solid supports, provided herein include the NCBI SNPs set forth in Table A, which are referenced by their respective locations in FIG. 1 and in SEQ ID NO:72.

TABLE A
SNCG NCBI Polymorphisms

| | | POSITION IN |
|---|---|---|
| NCBI SNP ID NO. | POLYMORPHISM | SEQ ID NO: 72 |
| rs1800714 | A/G | 560 |
| rs1802015 | A/T | 5112 |
| rs2131396 | G/T | 5421 |
| rs2131395 | A/G | 5648 |

### EXAMPLE 3

### SEQUENCING OF IDE, KNSL, TNFRSF6 AND LIPA CANDIDATE GENES

Genomic sequences were downloaded from the Human Genome Project public database. The exon-intron structure of each candidate gene was determined by querying the NCBI BLASTN search and alignment program with one or more cDNA sequences encoding the gene (Altschul et al. (1997) Nucleic Acids Res. 25:3389-3402). Based on this information, primers were designed to amplify regions of interest from genomic DNA and sequence them on both strands. These regions consisted of: (1) approximately 1 kb of 5′ flanking sequence 5′ to the beginning of exon 1, containing the putative promoter; (2) all exons plus 50-200 bp 5′ and 3′ flanking sequence for each one; and (3) ˜700 bp 3′ to the translation stop codon. When the final exon contained a 3′UTR>700 nt long, the region >700 nt 3′ to the stop codon was not amplified.

The genomic DNA samples were obtained from NIMH as described in Example 1. The desired regions were amplified by PCR using 30 ng each genomic DNA with the HotStarTaq Master Mix Kit (QIAGEN, Inc., Valencia, Calif.) and a final-concentration of 1 μm each of specific PCR primers (see Tables below) according to the manufacturer's protocol. The annealing temperature for different primers was varied as required. The reactions were purified using the QIAquick 96 PCR Purification Kit (QIAGEN, Inc., Valencia, Calif.) according to the manufacturer's protocol. PCR product yields were quantitated using the PicoGreen dsDNA Quantitation Kit (Molecular Probes, Inc., Eugene, Oreg.) according to the manufacturer's protocol. Sequencing reactions were performed with ABI PRISM BigDye” Terminators v3.0 Cycle Sequencing Kit (Applied Biosystems, Foster City, Calif.), using a modification of the manufacturer's protocol as follows. For each template and primer combination (see Tables, below), a mixture of 2 μl BigDye Mix, 4 μl 5×Sequencing Buffer, 8 μl H₂O, 2 μl primer (1.6 pmol/μl), and 4 μl PCR product (3 ng/μl) was subjected to 30 cycles of 10 s at 96° C., 5 s at 50° C. and 4 min at 60° C. The reactions were purified on a Centri-Sep 96 plate (Princeton Separations, Adelphia, N.J.) according to the manufacturer's protocol and analyzed in an ABI 3700 Automated DNA Sequencer (Applied Biosystems, Foster City, Calif.). Sequence data for each region in all samples were aligned using Sequencher software (GeneCodes Corp., Ann Arbor) and manually evaluated for the presence of polymorphisms.

IDE

The nucleotide sequence of the IDE gene in two sets of human genomic DNA samples was determined. The “D10-Top10” set was derived from 10 members of three families with the highest scores for AD linkage to genetic marker D10S583. The “D10-E16” set was derived from sixteen genomic DNA samples from four families showing the highest combined LOD scores for linkage with LOAD at 6 markers (D10S564, D10S583, D10S1710, D10S566, D10S1671 and D10S1741). Each set contained individuals both affected and unaffected with AD.

Twenty-five exons have been identified in the human IDE gene by comparison of cDNA clones containing the IDE coding sequence (CDS) with genomic sequence data. The cDNA corresponding to GenBank accession No. NM_{—}004969 (Affholter etal. (1988) Science 242:1415-1418) contains a 5′UTR having 57 nucleotides (nt) 5′ to the translation initiation codon, the complete 3060-nt coding sequence (CDS) and 162 nucleotides of 3′UTR. Genomic DNA sequence corresponding to GenBank accession No. AL356128.15 (corresponding to Chromosome 10 BAC clone RP11-366I13) was used for primer design.

Based on this information, primers were designed to amplify and sequence exons 1 through 25 and at least approximately 50-200 bp 5′ and 3′ to the exon boundaries indicated by the above CDNA sequences. Approximately 1.4 kb of the presumed promoter-containing region 5′ to exon 1 was also amplified and sequenced. Approximately 400 bp 3′ to the second stop codon in exon 25 were amplified and sequenced (FIG. 2). The primers used for amplifying IDE genomic fragments corresponding to the 5′ regulatory region and the 25 exons and their corresponding sequencing primers are shown in Table 3.

TABLE 3

| | | | |
|---|---|---|---|
| | | SEQ | |
| | | ID | |
| NAME | SEQUENCE | NO | PURPOSE |

| | | | |
|---|---|---|---|
| IDE.Pro.p1 | CTCCATTCCGCTTACCCAA | 74 | PCR |
| IDE.Pro.p2 | AAGAGGTCCCCCAAATTGTA | 75 | PCR |
| IDE.pro.s1 | GAGGACTGAGCGGAAGGT | 76 | sequencing |
| IDE.pro.s2 | GGACTAAGGACACCGTTT | 77 | sequencing |
| IDE.pro.s3 | TGGCGACGTGTGTCTGAC | 78 | sequencing |
| IDE.pro.s4 | ACCACTGAAGCCGACTGA | 79 | sequencing |
| IDE.pro.s5 | GGCGAGGACGGTGAAGA | 80 | sequencing |
| IDE.pro.s6 | GTCCTCGCCTGCGTCCT | 81 | sequencing |
| IDE.pro.s7 | AGGCACCCCCACTGACT | 82 | sequencing |
| IDE.exon01.p1 | TGCAGGCCGGGTGACTCT | 83 | PCR |
| IDE.exon01.p2 | CGCCCCCTCACAGTCAGACA | 84 | PCR |
| IDE.exon01.s1 | CGGTGGCGGCTTTAAGT | 85 | sequencing |
| IDE.exon01.s2 | CGTCGCCACCCTCACAA | 86 | sequencing |
| IDE.exon02.p1 | TTGCTGCTAGGTCACCTTA | 87 | PCR |
| IDE.exon02.p2 | CTATCTCAAATCCCTATCCA | 88 | PCR |
| IDE.exon02.s1 | CTTTACGAGGGTTCTGGTA | 89 | sequencing |
| IDE.exon02.s2 | GGAGGAAAGGAATGACTG | 90 | sequencing |
| IDE.exon03.p1 | AGTCCTCATCACATGCTTTATA | 91 | PCR |
| IDE.exon03.p2 | GGGAAACTAATGTTCAGAGA | 92 | PCR |
| IDE.exon03.s1 | ATGGAAAGGGCTGCTGAA | 93 | sequencing |
| IDE.exon03.s2 | CAAGAGCAGGGACTAGAA | 94 | sequencing |
| IDE.exon04.p1 | TAACTGCTCTGCTCCTTAAACG | 95 | PCR |
| IDE.exon04.p2 | TCATCTCTGGGGTAGATCAAC | 96 | PCR |
| IDE.exon04.s1 | CTGAATTGTAAAATATGCCA | 97 | sequencing |
| IDE.exon04.s2 | GCATTTACCTCTGTTCAAC | 98 | sequencing |
| IDE.exon05.p1 | AATCCCCACCTATGTATTCT | 99 | PCR |
| IDE.exon05.p2 | AAAGACACTTTTAATTCCGTAG | 100 | PCR |
| IDE.exon05.s1 | TCCCCACCTATGTATTCTC | 101 | sequencing |
| IDE.exon05.s2 | TTAAAATGTTGCTGGTTTG | 102 | sequencing |
| IDE.exon06.p1 | AGGTGGGAGGATCAATTCA3′ | 103 | PCR |
| IDE.exon06.p2 | GAAGGCAATTCTCTGTCTATGT | 104 | PCR |
| IDE.exon06.s1 | CACAACATAAATAATGAAACC | 105 | sequencing |
| IDE.exon06.s2 | TTTTCACATTTTCCTTTCAG | 106 | sequencing |
| IDE.exon07.p1 | CGAAACCCCATCTCTATCT | 107 | PCR |
| IDE.exon07.p2 | ATCATTTTCTGTGGCAATTGA | 108 | PCR |
| IDE.exon07.s1 | GTGCCAACAACCACCATC | 109 | sequencing |
| IDE.exon07.s2 | GGATTCTCCCATGAAAATC | 110 | sequencing |
| IDE.exon08.p1 | ACACTTAAAAACCATTCAGTCC | 111 | PCR |
| IDE.exon08.p2 | GCCCAGCCAGTAGTCTACTC | 112 | PCR |
| IDE.exon08.s1 | GGGGGAAGAATCATCCAT | 113 | sequencing |
| IDE.exon08.s2 | CCCACTTCTGCACCATC | 114 | sequencing |
| IDE.exon09.p1 | GCCTCTTCCCATACATACAAT | 115 | PCR |
| IDE.exon09.p2 | AACACCCTTTCTGTCACATATC | 116 | PCR |
| IDE.exon09.s1 | GCCCCAAGTAGCAAATCA | 117 | sequencing |
| IDE.exon09.s2 | CCATCTTCCACTTATCTGA | 118 | sequencing |
| IDE.exon10.p1 | ATCGCAGGTAAAAGACTATC | 119 | PCR |
| IDE.exon10.p2 | AGTTTCAACAACTTAGGCTATC | 120 | PCR |
| IDE.exon10.s1 | GCCCTGTTGCTACAAAGT | 121 | sequencing |
| IDE.exon10.s2 | TTCTAAATCAAGCATAGCC | 122 | sequencing |
| IDE.exon11.p1 | AAGAGGGATGGGTATAGATTA | 123 | PCR |
| IDE.exon11.p2 | TTTTAACCAGAAACATTCGTG | 124 | PCR |
| IDE.exon11.s1 | TGGTGTTGCTTCATTATAC | 125 | sequencing |
| IDE.exon11.s2 | GGTAGGAAAGAGACAACGA | 126 | sequencing |
| IDE.exon12.p1 | TATGGCATGAGGCACAACC | 127 | PCR |
| IDE.exon12.p2 | GAATCTCTCCTGCGAGCACT | 128 | PCR |
| IDE.exon12.s1 | GCTCCCTATCTACTTGTTGA | 129 | sequencing |
| IDE.exon12.s2 | AACTAAAGGATAACAGGGAC | 130 | sequencing |
| IDE.exon13.p1 | CAAAGAAAGTGATTCCCTA | 131 | PCR |
| IDE.exon13.p2 | TGATCTGCATATGGACCAGT | 132 | PCR |
| IDE.exon13.s1 | TGTGAGCCTCCCCAAATC | 133 | sequencing |
| IDE.exon13.s2 | ATGGAAAACAATGCTGTG | 134 | sequencing |
| IDE.exon14.p1 | TTAGCCAGGATGGTCTCAC | 135 | PCR |
| IDE.exon14.p2 | CCTAGTGTCCAGAAAGACGA | 136 | PCR |
| IDE.exon14.s1 | GCCTCTCCTTTTCTAGGTG | 137 | sequencing |
| IDE.exon14.s2 | CAGCAACTCCAGAACAAA | 138 | sequencing |
| IDE.exon15.p1 | AACCTAAATCAGGGTCCAAT | 139 | PCR |
| IDE.exon15.p2 | TCTTCACTACTACCAGTGCG | 140 | PCR |
| IDE.exon15.s1 | ATGAGAAAATATGATTAGCC | 141 | sequencing |
| IDE.exon15.s2 | AGCCTCAAGAATTCAACC | 142 | sequencing |
| IDE.exon16.p1 | AGGTGTCCACCAGAGTCTACTA | 143 | PCR |
| IDE.exon16.p2 | TGAGGTTGCAGTGAACTGAA | 144 | PCR |
| IDE.exon16.s1 | AATGCCACAGGGTTATGA | 145 | sequencing |
| IDE.exon16.s2 | TTACATGTCTGTTTTGGGAT | 146 | sequencing |
| IDE.exon17.p1 | TATCATTTGTTTGGGTTGGG | 147 | PCR |
| IDE.exon17.p2 | GGGGATGGCTTTATAGCGT | 148 | PCR |
| IDE.exon17.s1 | ACAGCCAGCTTTAATAGA | 149 | sequencing |
| IDE.exon17.s2 | CAGAAAGGGGAAAGAAGG | 150 | sequencing |
| IDE.exon18.p1 | TTGAATCCAGGCTGTCTAAT | 151 | PCR |
| IDE.exon18.p2 | AATGGGAAAAATCCAAATA | 152 | PCR |
| IDE.exon18.s1 | TCCACAGAGCAAGAGGGT | 153 | sequencing |
| IDE.exon18.s2 | GGGAAAACTAGGTGAAGTG | 154 | sequencing |
| IDE.exon19.p1 | TCAAGTACCCGTCAATTC | 155 | PCR |
| IDE.exon19.p2 | TTTTCCAGCATGAGCATCAG | 156 | PCR |
| IDE.exon19.s1 | GAGAGGTTCCCAAGTAAA | 157 | sequencing |
| IDE.exon19.s2 | TTTCTTTCACCACTACCATA | 158 | sequencing |
| IDE.exon20.p1 | GGCTTGGGATATAAAGACGA | 159 | PCR |
| IDE.exon20.p2 | TGATGAGAGGGAACTTAGTGCT | 160 | PCR |
| IDE.exon20.s1 | AAGGTTTTCTCAAGTCTCTC | 161 | sequencing |
| IDE.exon20.s2 | AACTCCAGGCTTCTCTAC | 162 | sequencing |
| IDE.exon21.p1 | GGGTAGATCCAGGTCTAGGT | 163 | PCR |
| IDE.exon21.p2 | TGACAAGATGGGAGTTTGAA | 164 | PCR |
| IDE.exon21.s1 | ACCCCAACTCCCCAAATA | 165 | sequencing |
| IDE.exon21.s2 | TTTATTTGCTCAGACAGGTT | 166 | sequencing |
| IDE.exon22.p1 | AAAGTCATTAAGCGTTTGTG | 167 | PCR |
| IDE.exon22.p2 | TATAAAAATTACCCAGACGTGT | 168 | PCR |
| IDE.exon22.s1 | CTGACCTCAAGTGATCCTT | 169 | sequencing |
| IDE.exon22.s2 | CCACTGCACTCCAGCGT | 170 | sequencing |
| IDE.exon23.p1 | TGTGATGCTGTGGCTAGCTC | 171 | PCR |
| IDE.exon23.p2 | CAAACTTGCTTCCTCGTGT | 172 | PCR |
| IDE.exon23.s1 | TTATCTCCAGTTTCCGTTC | 173 | sequencing |
| IDE.exon23.s2 | GGGAAGCATCTTTTCTATA | 174 | sequencing |
| IDE.exon24.p1 | TTTTAGTTCCCAATGAACAAGT | 175 | PCR |
| IDE.exon24.p2 | GGTAGCAGGCAACCTTTAG | 176 | PCR |
| IDE.exon24.s1 | CCCAAGAAGTGGAGGTTGTA | 177 | sequencing |
| IDE.exon24.s2 | CAGGCAACCTTTAGAGGAT | 178 | sequencing |
| IDE.exon25.p1 | TAAAAAGATTAAAACCATGCC | 179 | PCR |
| IDE.exon25.p2 | GTACAGACCAATTCACGACCC | 180 | PCR |
| IDE.exon25.s1 | ATGTAATGTTTTCCACTGAT | 181 | sequencing |
| IDE.exon25.s2 | CAGAAGAAAGGTCAGCAGA | 182 | sequencing |
| IDE.exon25.s3 | CATTCATTTTAAGCATTGT | 183 | sequencing |
| IDE.exon25.s4 | GTATGGTCCCAGTGTCCG | 184 | sequencing |
| IDE.exon25.s6 | CAAAACTCTGAAGATTCCC | 185 | sequencing |

Polymorphic regions (such as SNPs, and the like) were discovered by comparing the sequenced samples to reference IDE genomic sequences (SEQ ID NO:186 and the reverse complement of nucleotides 1-130,000 of SEQ ID NO:484) and identifying nucleotides that varied from the reference nucleotide sequence at specific positions (see Tables 4 and 4-B). SEQ ID NO:186 represents the reverse complement of a 128,034 nucleotide sequence corresponding to NCBI Accession# AL356128.15. Table 4 shows the polymorphic regions that were identified, including previously identified polymorphic regions (set forth as a yes in the public database column), and the type of nucleotide polymorphic change detected relative to the IDE reference genomic sequence set forth as SEQ ID NO:186.

TABLE 4
(IDE Polymorphic Regions)

| | | COMPLEMENT | | |
|---|---|---|---|---|
| | | STRAND | | |
| | POSITION | POSITION IN | | NUCLEOTIDE |
| | IN SEQ ID | SEQ ID | PUBLIC | CHANGE |
| POLYMORPHISM | NO: 186 | NO: 484 | DATABASE? | DETECTED |
| US-945 | 2456 | 121239 | NO | T → G |
| US-122 | 3279 | 120416 | NO | T → C |
| 1.e5′UTR-51 | 3407 | 120288 | NO | C → T |
| 3i.D + 42 | 42943 | 80752 | NO | T → C |
| 4i.A-7 | 62498 | 61270 | NO | T → C |
| 8i.D + 149 | 69586 | 54182 | NO | T → C |
| 18i.D + 98 | 107395 | 16373 | NO | G → A |
| 20i.D + 249 | 112114 | 11654 | NO | G → A |
| 22i.D + 302 | 116662 | 7106 | YES | T → A |

Upstream Sequence

Two polymorphic regions (i.e., SNPs) were discovered upstream of the transcription start site of the IDE gene, US-945 and US-122, which are located 945 and 122 nucleotides, respectively, before exon 1 of the IDE gene (positions 2456 and 3279 of SEQ ID NO:186). These SNPS are located in putative promoter or enhancer regions of the IDE gene and as such a nucleotide change may affect the expression of the IDE gene, e.g., level, response to stimulatory or inhibitory molecules, and the like.

5′ UTR Sequence

The polymorphic region corresponding to the SNP labeled 1.e5′UTR-51 is located 51 nucleotides upstream of the translation start codon in the IDE cDNA. This site may affect the level of expression of the IDE transcript.

Intervening Sequences

The polymorphic regions corresponding to SNPs labeled as 3i.D+42, 4i.A-7, 8i.D+149, 18i.D+98, 20i.D+249, and 221.D+302 1iD+41 are contained in introns. For example, 3i.D+42 is located 42 nucleotides after exon 3, 4i.A-7 is located 7 nucleotides before exon 5, 8i.D+149 is located 149 nucleotides after exon 8, 18i.D+98 is located 98 nucleotides after exon 18, 20i.D+249, is located 249 nucleotides after exon 20 and 22i.D+302 is located 302 nucleotides after exon 22. These SNPS may affect splicing of the IDE RNA transcript, or the like.

Table 4-B shows additional IDE polymorphic regions that were identified, including previously identified polymorphic regions (set forth as a yes in the public database column), and the type of nucleotide polymorphic change detected relative to the IDE reference genomic sequence that corresponds to the reverse complement of nucleotides 1 through approximately 130,000 set forth in SEQ ID NO:484. Thus, the strand set forth in SEQ ID NO:484 contains the complementary nucleotides to the actual polymorphic nucleo tide regions on the coding strand set forth in Table 4-B. For example, the polymorphism located in Table 4-B at position 21270 corresponding to T-G (e.g., T/G) is referred to in SEQ ID NO:484 as either A or C.

TABLE 4-B

| | COMPLEMENT | | NUCLEOTIDE |
|---|---|---|---|
| POLYMORPHISM | STRAND POSITION IN SEQ | PUBLIC | CHANGE |
| NAME | ID NO: 484 | DATABASE? | DETECTED |
| IDE_25i.1 | 820 | NO | A → T |
| IDE_23i.2 | 7066 | NO | A → G |
| IDE_21i.1 | 11758 | YES | T → C |
| IDE_17i.1 | 21270 | NO | T → G |
| IDE_16i.1 | 22225 | NO | A → T |
| IDE_15i.1 | 29294 | NO | C → T |
| IDE_13e.1 | 33452 | NO | T → G |
| IDE_12i.1 | 33708 | NO | G → A |
| IDE_11i.1 | 36982 | NO | C → T |
| IDE_7i.1 | 54862 | YES | A → G |
| IDE_4i.1 | 77786 | NO | C → A |
| IDE_3i.2 | 80594 | NO | G → A |
| IDE_1i.D + 35348 | 84792 | YES | C → T |
| IDE_1i.D + 35143 | 84997 | NO | T → G |
| IDE_1i.D + 33458 | 86682 | NO | C → T |
| IDE_1i.D + 33283 | 86857 | NO | T → A |
| IDE_1i.D + 31631 | 88511 | NO | A → G |
| IDE_1i.D + 29704 | 90437 | NO | G → T |
| IDE_1i.D + 29548 | 90593 | NO | G → A |
| IDE_1i.D + 28490 | 91650 | NO | T → C |
| IDE_1i.D + 28270 | 91870 | NO | A → G |
| IDE_1i.D + 28262 | 91878 | NO | G → A |
| IDE_1i.D + 28129 | 92011 | NO | C → T |
| IDE_1i.D + 26522 | 93618 | NO | T → C |
| IDE_1i.D + 25796 | 94344 | NO | T → C |
| IDE_1i.D + 25426 | 94714 | NO | A → G |
| IDE_1i.D + 24469 | 95671 | NO | T → G |
| IDE_1i.D + 23816 | 96324 | NO | G → A |
| IDE_1i.D + 22838 | 97302 | NO | G → A |
| IDE_1i.D + 22770 | 97370 | NO | G → A |
| IDE_1i.D + 21887 | 98253 | NO | T → C |
| IDE_1i.D + 21864 | 98276 | YES | C → T |
| IDE_1i.D + 21755 | 98385 | NO | A → G |
| IDE_1i.D + 21494 | 98646 | NO | A → T |
| IDE_1i.D + 21326 | 98814 | NO | G → A |
| IDE_1i.D + 20543 | 99597 | NO | T → C |
| IDE_1i.D + 19762 | 100378 | NO | T → C |
| IDE_1i.D + 19111 | 101029 | NO | A → G |
| IDE_1i.D + 18875 | 101265 | NO | T → C |
| IDE_1i.D + 17675 | 102465 | NO | C → G |
| IDE_1i.D + 16851 | 103289 | NO | G → T |
| IDE_1i.D + 16173 | 103967 | NO | C → T |
| IDE_1i.D + 14347 | 105793 | NO | A → G |
| IDE_1i.D + 14064 | 106076 | NO | G → T |
| IDE_1i.D + 13687 | 106453 | YES | C → T |
| IDE_1i.D + 13540 | 106600 | YES | A → G |
| IDE_1i.D + 13145 | 106995 | NO | G → A |
| IDE_1i.D + 12289 | 107851 | NO | C → T |
| IDE_1i.D + 11706 | 108434 | NO | G → C |
| IDE_1i.D + 11044 | 109096 | NO | C → T |
| IDE_1i.D + 10741 | 109399 | NO | C → T |
| IDE_1i.D + 10657 | 109483 | NO | G → T |
| IDE_1i.D + 9270 | 110870 | NO | G → A |
| IDE_1i.D + 8951 | 111189 | NO | A → G |
| IDE_1i.D + 8168 | 111972 | NO | G → A |
| IDE_1i.D + 7513 | 112627 | NO | A → T |
| IDE_1i.D + 7511 | 112629 | YES | A → T |
| IDE_1i.D + 7509 | 112631 | YES | A → T |
| IDE_1i.D + 6733 | 113407 | NO | G → C |
| IDE_1i.D + 5696 | 114444 | YES | C → G |
| IDE_1i.D + 5658 | 114482 | NO | G → C |
| IDE_1i.D + 4667 | 115473 | NO | Deletion of C |
| IDE_1i.D + 3459 | 116681 | NO | T → G |
| IDE_1i.D + 2914 | 117226 | NO | A → T |
| IDE_1i.D + 2540 | 117600 | NO | A → G |
| IDE_1i.D + 2338 | 117802 | YES | C → T |
| IDE_1i.D + 1917 | 118223 | NO | C → G |
| IDE_1i.1 | 120011 | NO | C → T |
| IDE_us-1966 | 122260 | NO | T → C |
| IDE_us-2871 | 123165 | NO | T → C |
| IDE_us-3130 | 123424 | NO | C → T |
| IDE_us-4058 | 124352 | NO | T → C |
| IDE_us-4207 | 124501 | NO | G → A |
| IDE_us-4398 | 124692 | NO | T → C |
| IDE_us-4819 | 125113 | NO | A → T |
| IDE_us-4865 | 125159 | NO | C → T |
| IDE_us-6274 | 126568 | NO | C → G |
| IDE_us-6872 | 127166 | NO | G → C |
| IDE_us-7304 | 127598 | NO | A → G |
| IDE_us-7306 | 127600 | NO | A → G |
| IDE_us-7315 | 127609 | NO | A → G |
| IDE_us-7320 | 127614 | NO | A → G |
| IDE_us-7329 | 127623 | NO | A → G |
| IDE_us-7368 | 127662 | NO | T → C |
| IDE_us-7759 | 128053 | NO | G → A |
| IDE_us-7970 | 128261 | NO | -TAAA- Repeated |
| | | | 6, 7 or 8 times |
| IDE_us-7995 | 128289 | NO | T → A |
| IDE_us-7997 | 128291 | NO | A → C |
| IDE_us-8099 | 128393 | NO | A → C |
| IDE_us-9150 | 129444 | NO | A → G |

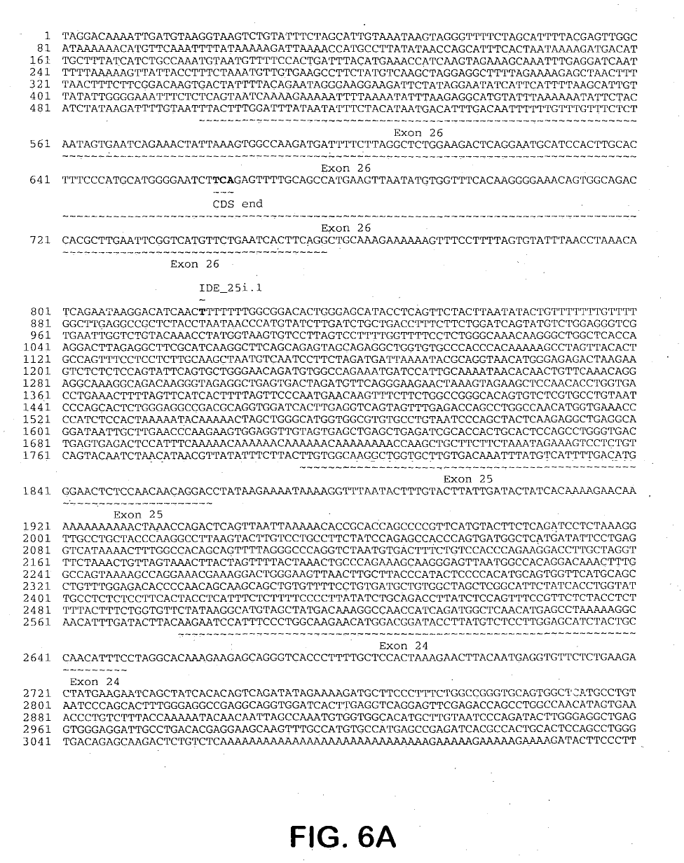

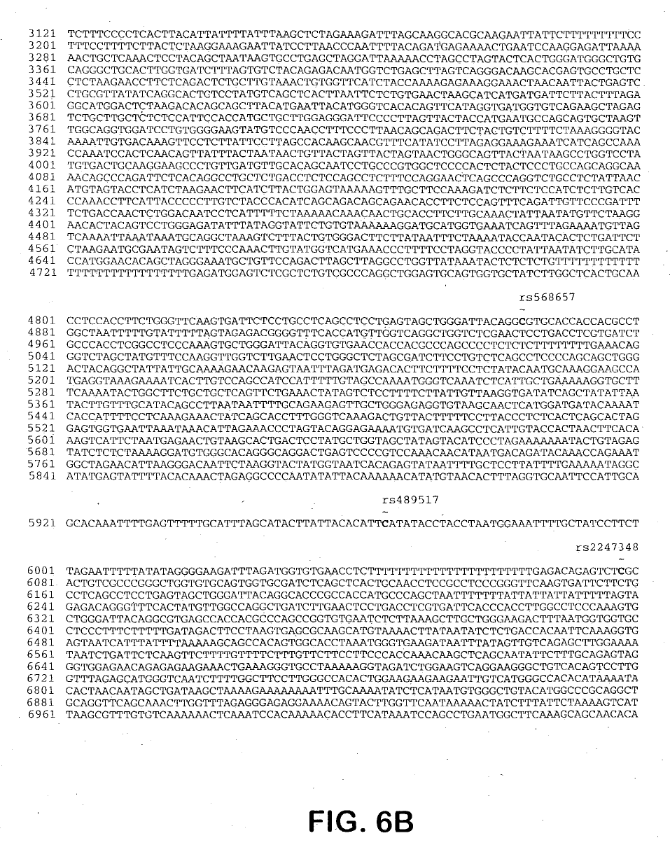

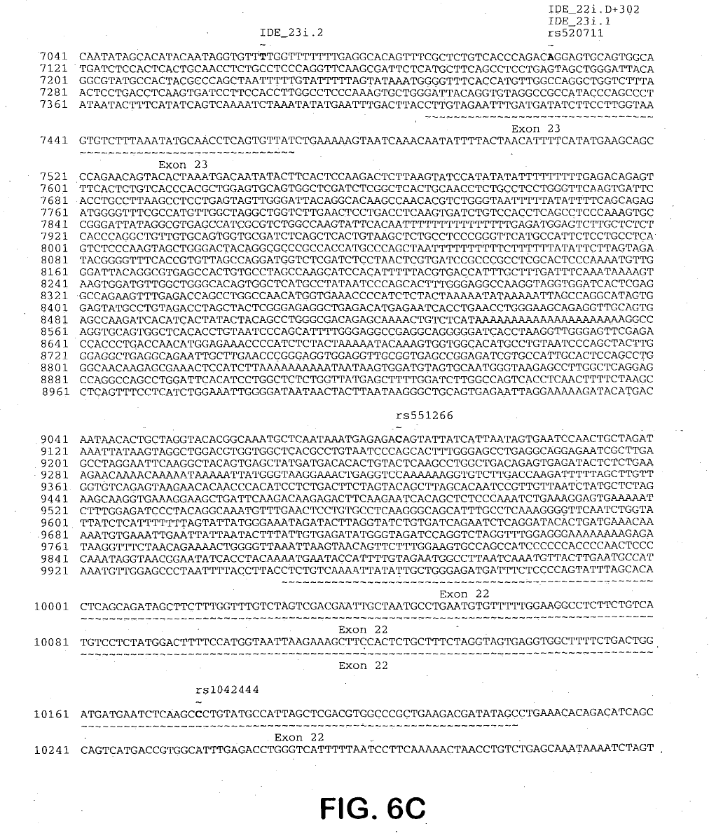

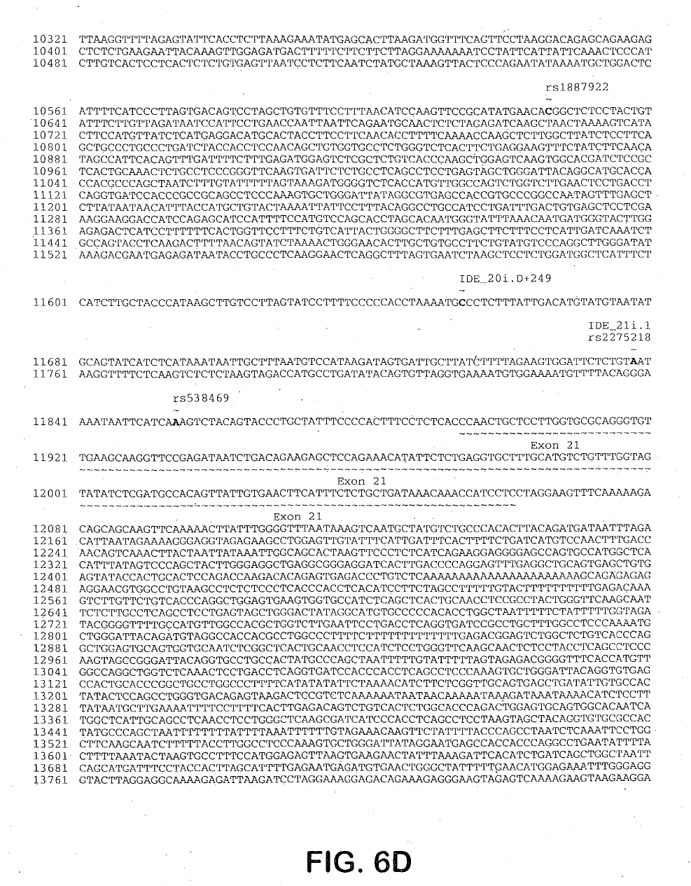

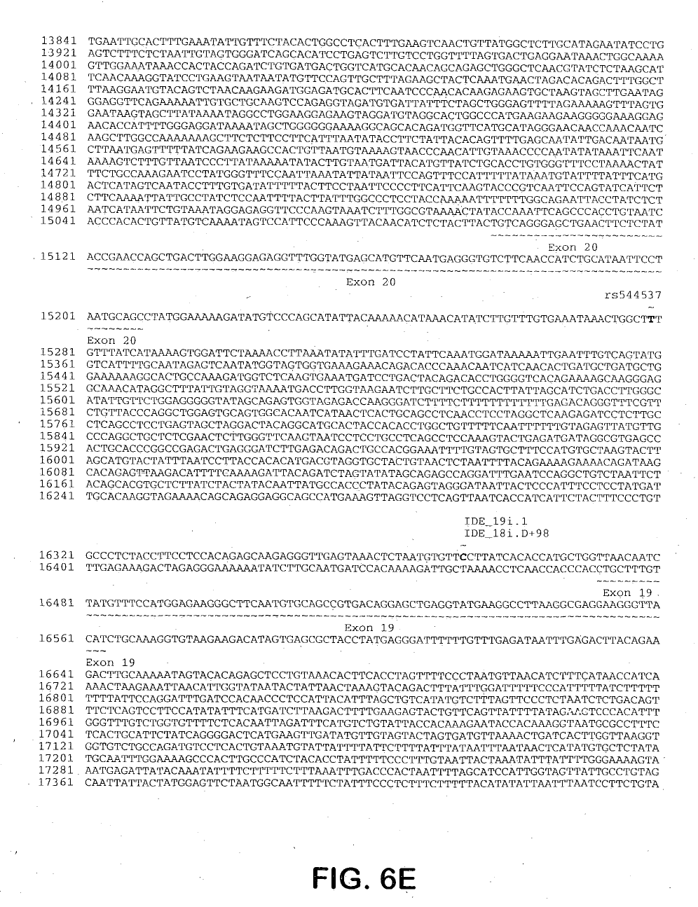

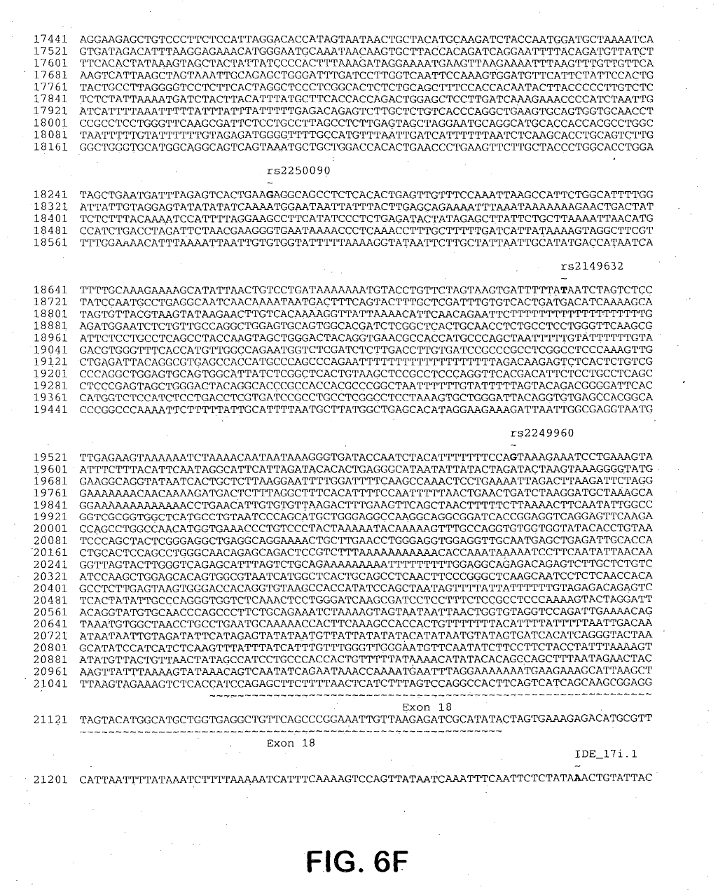

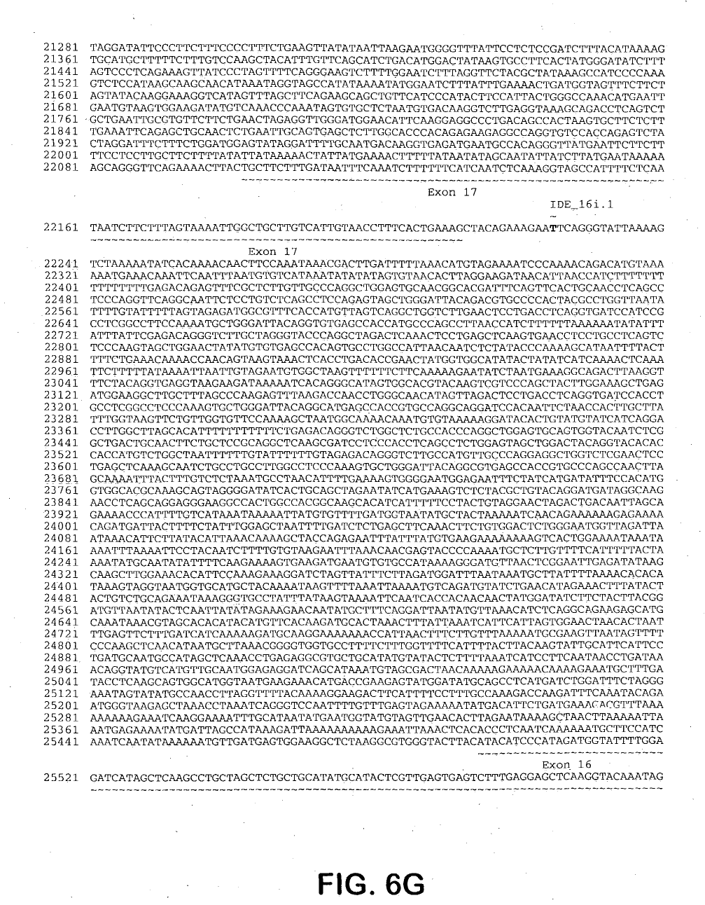

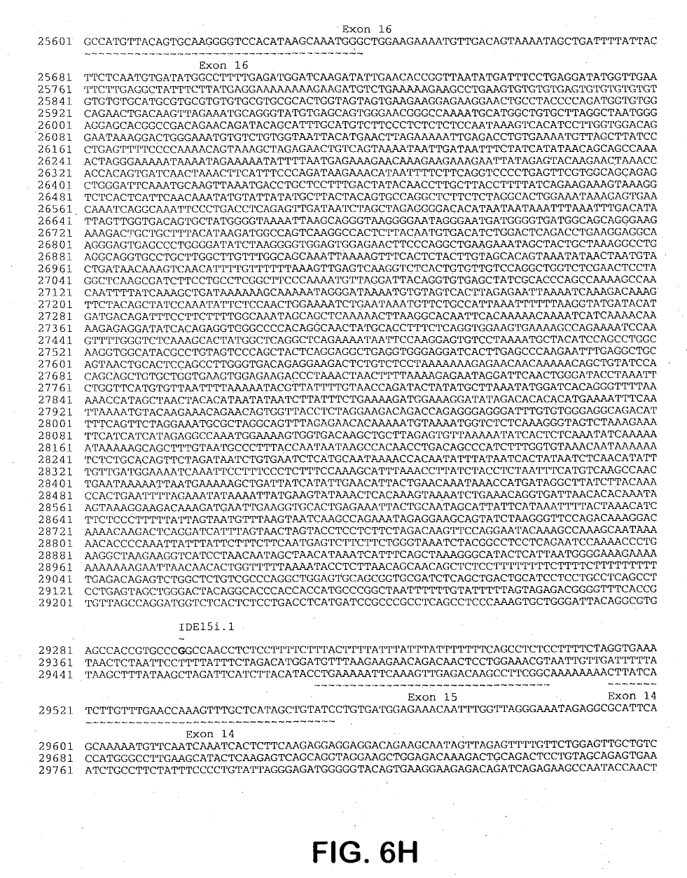

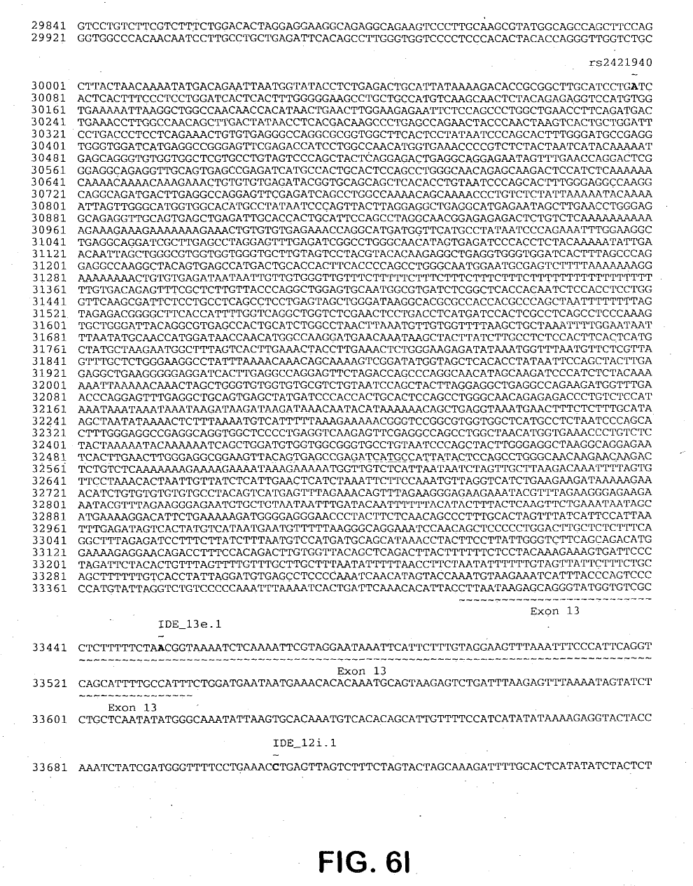

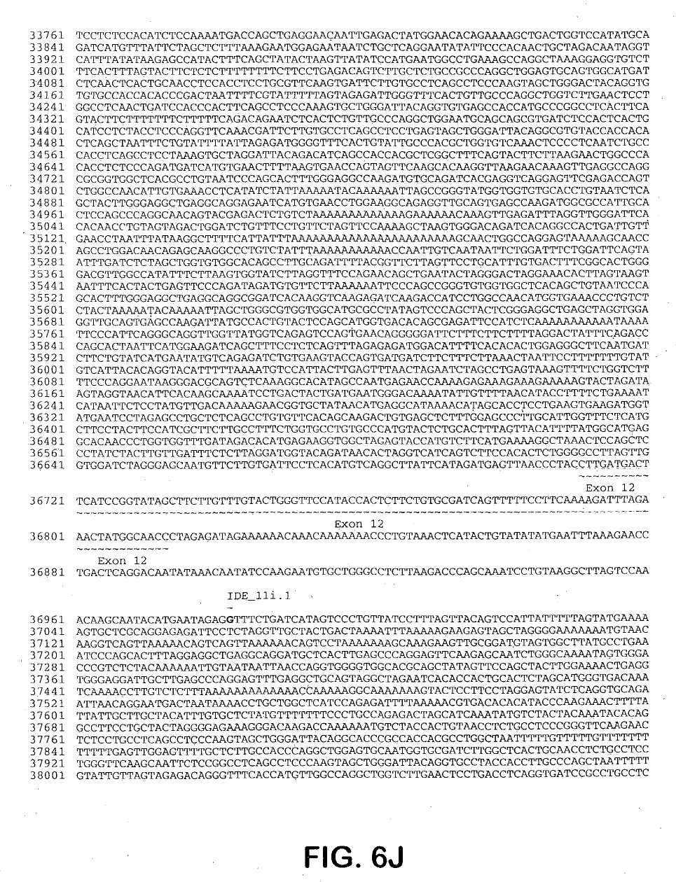

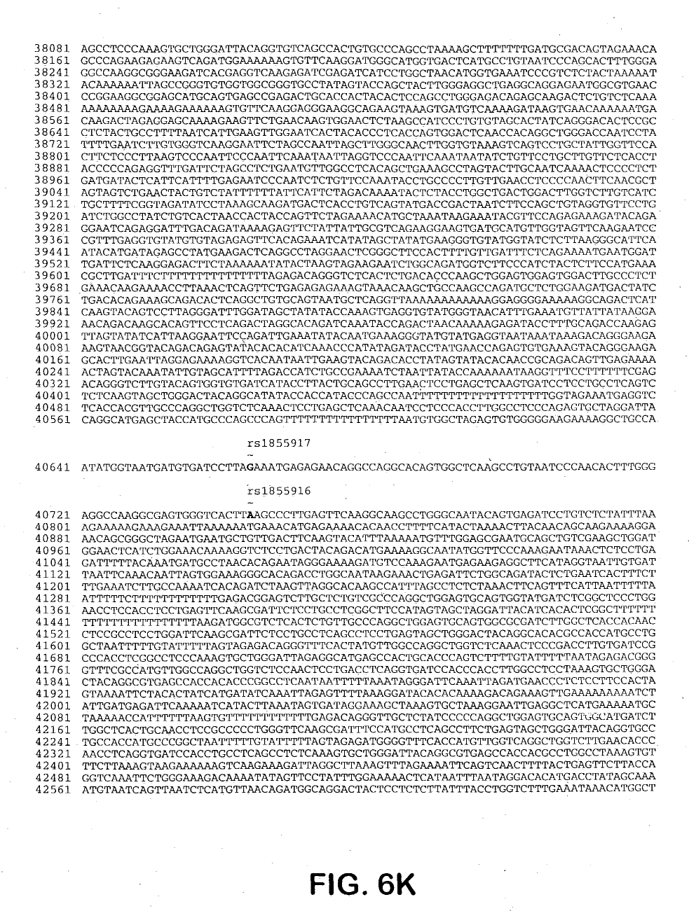

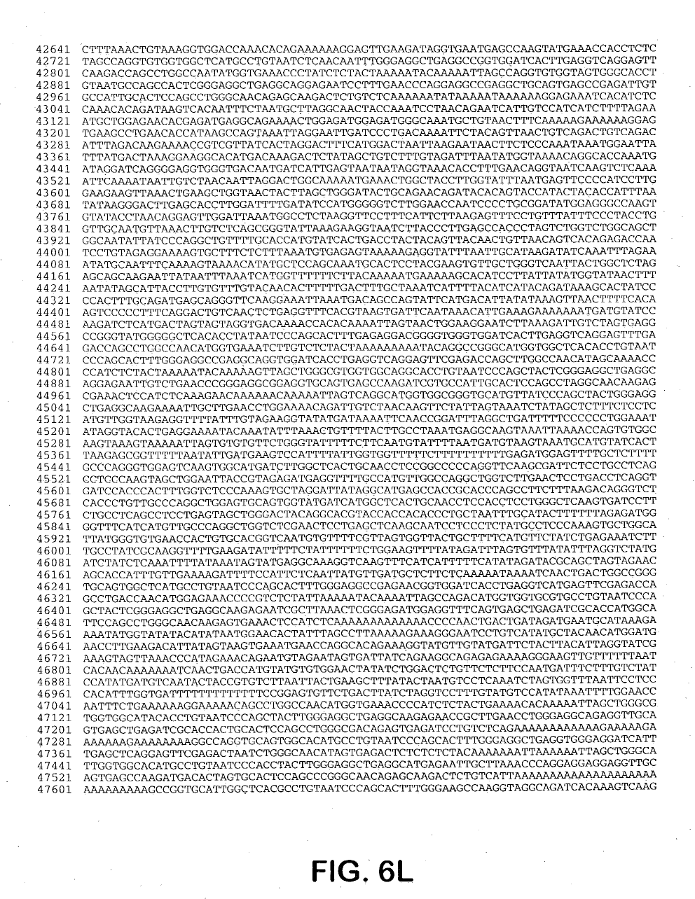

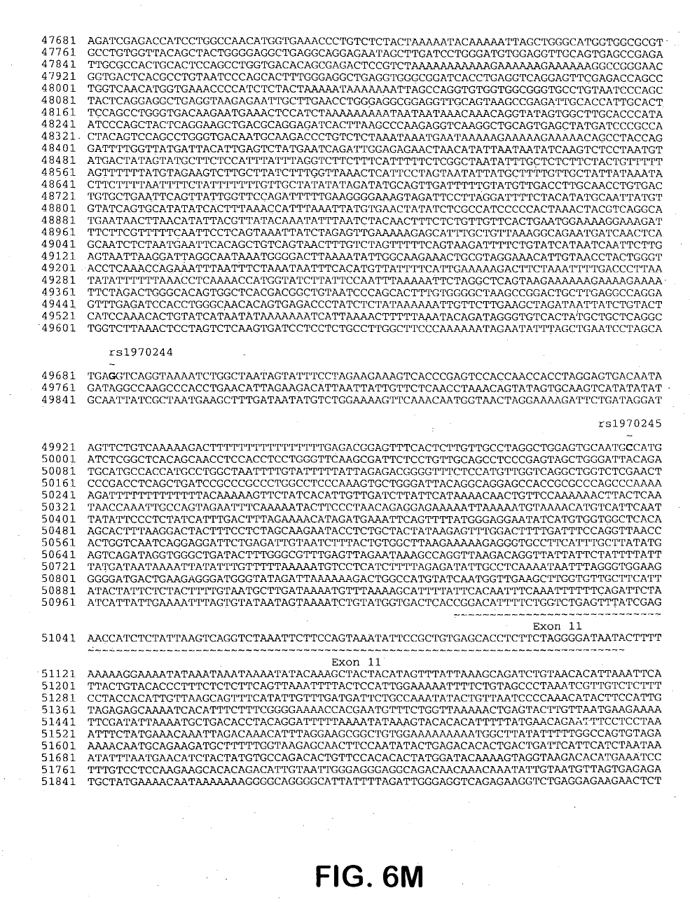

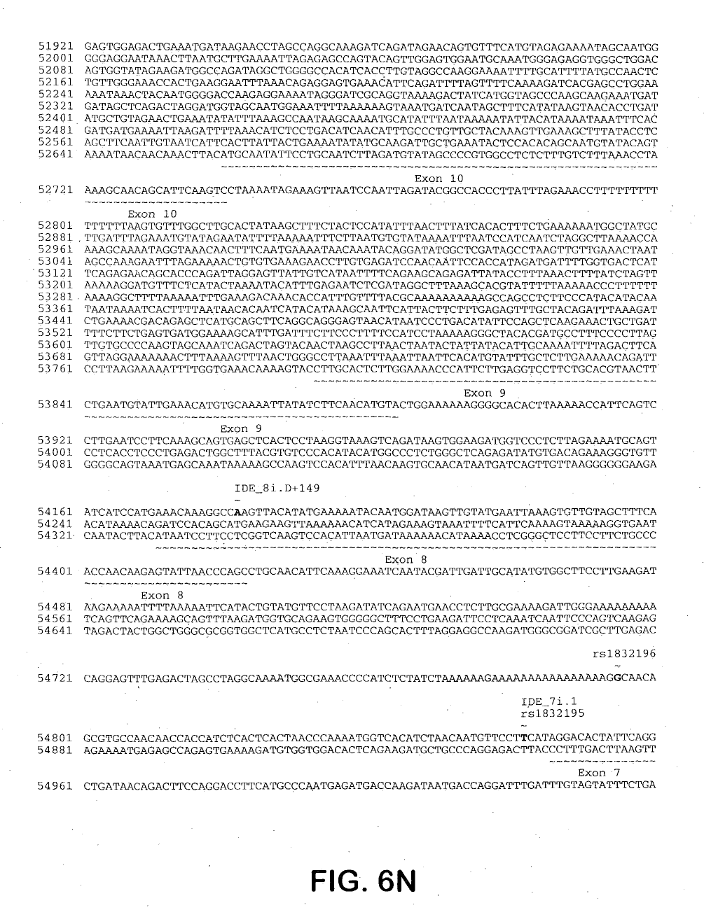

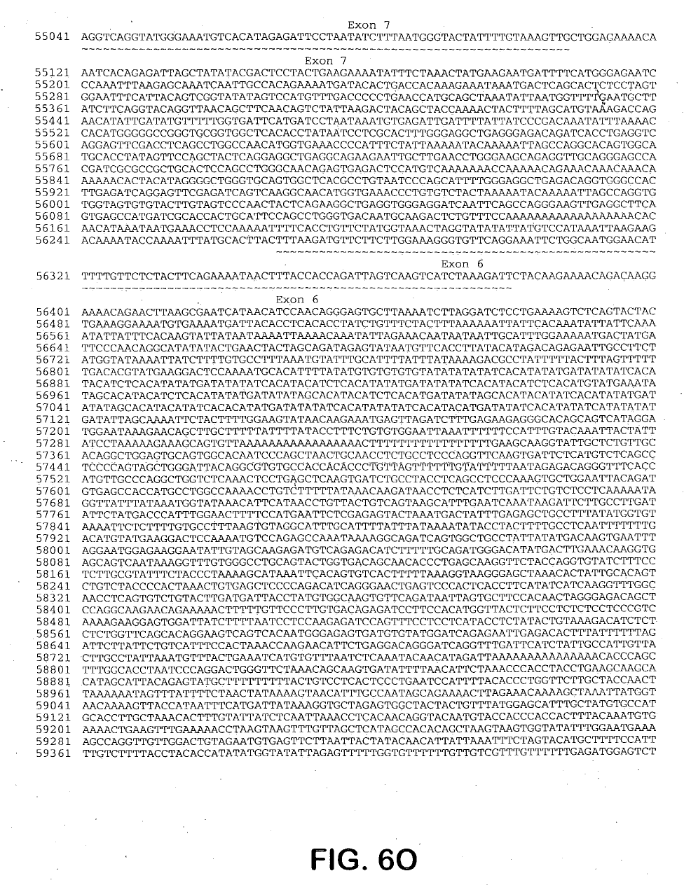

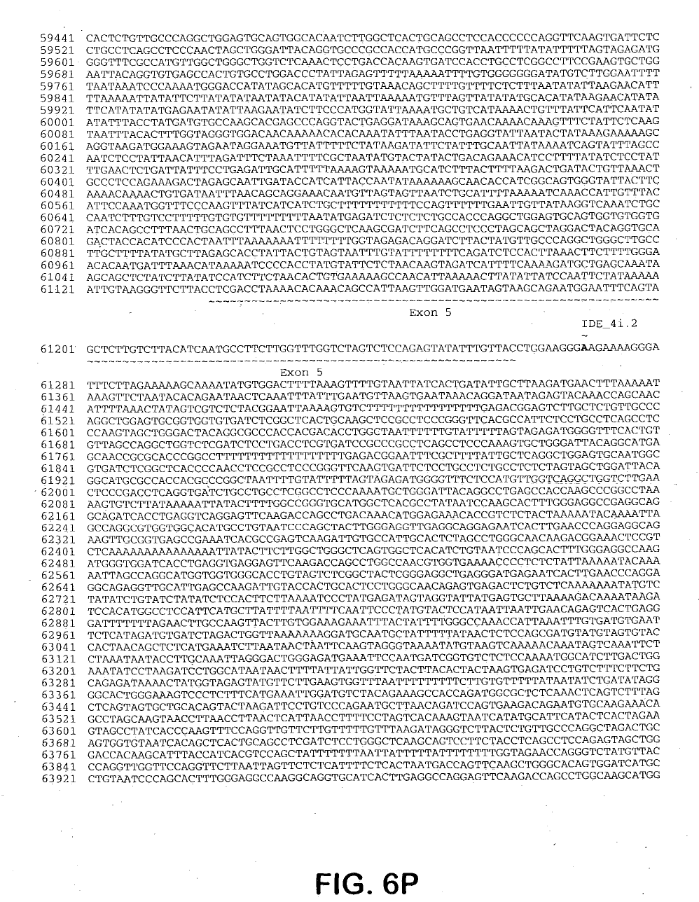

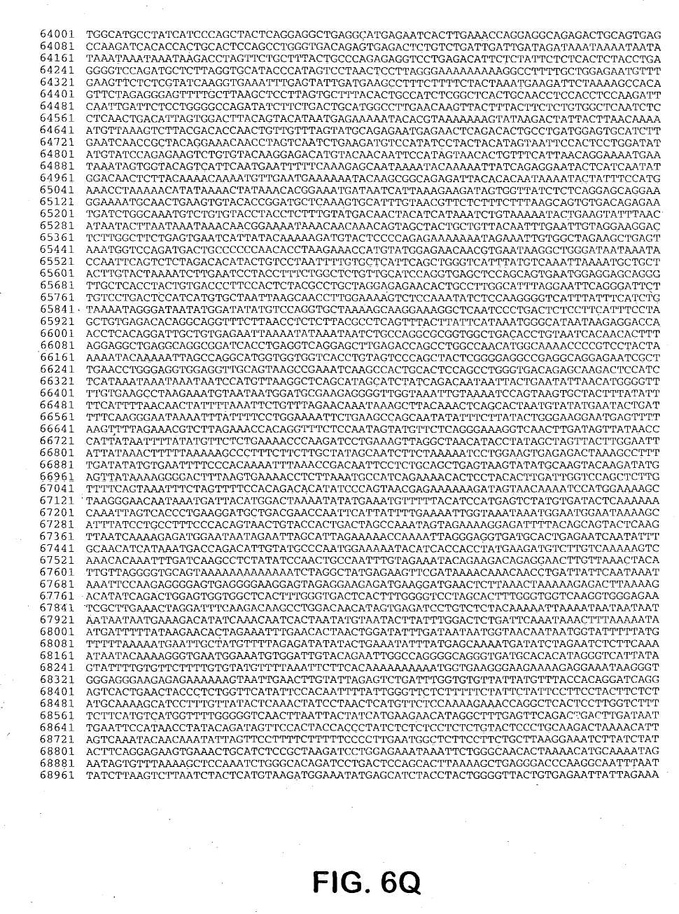

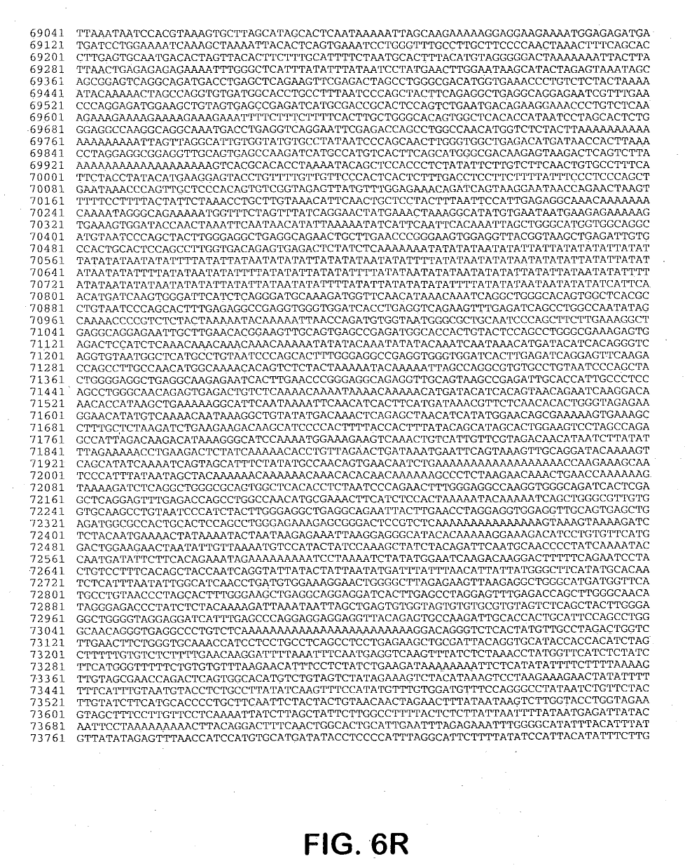

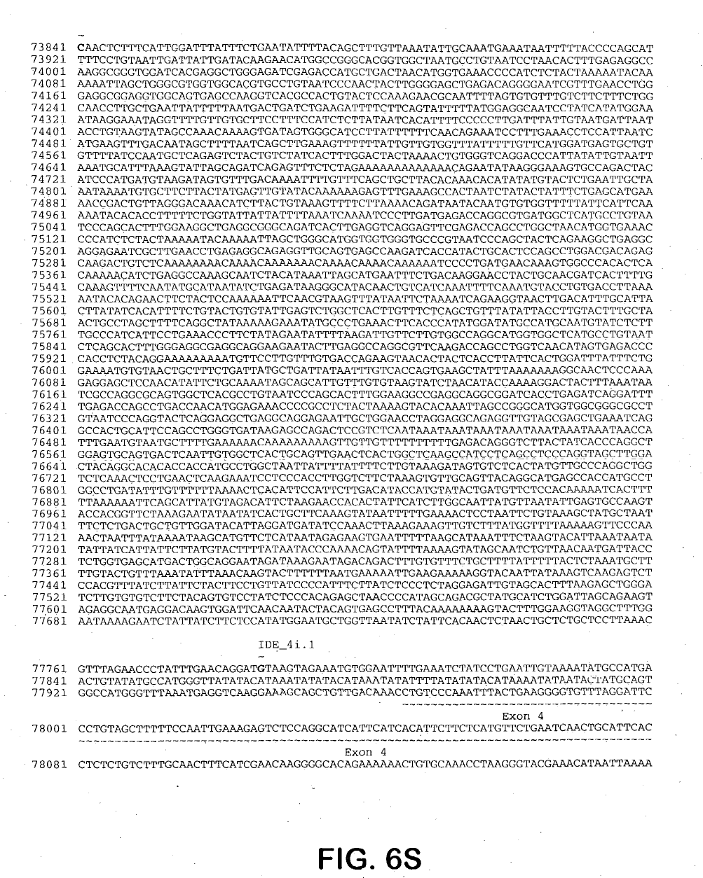

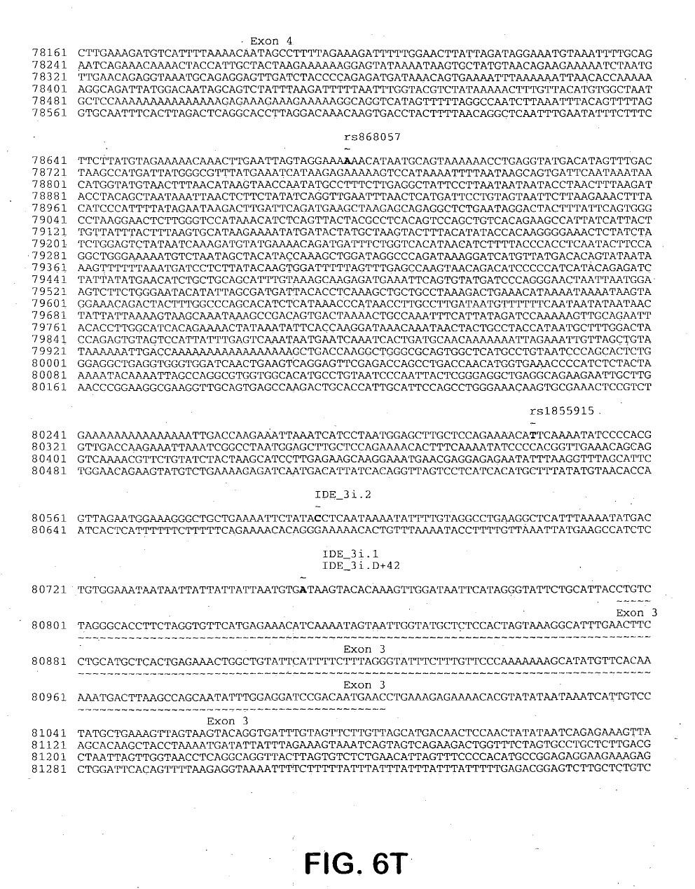

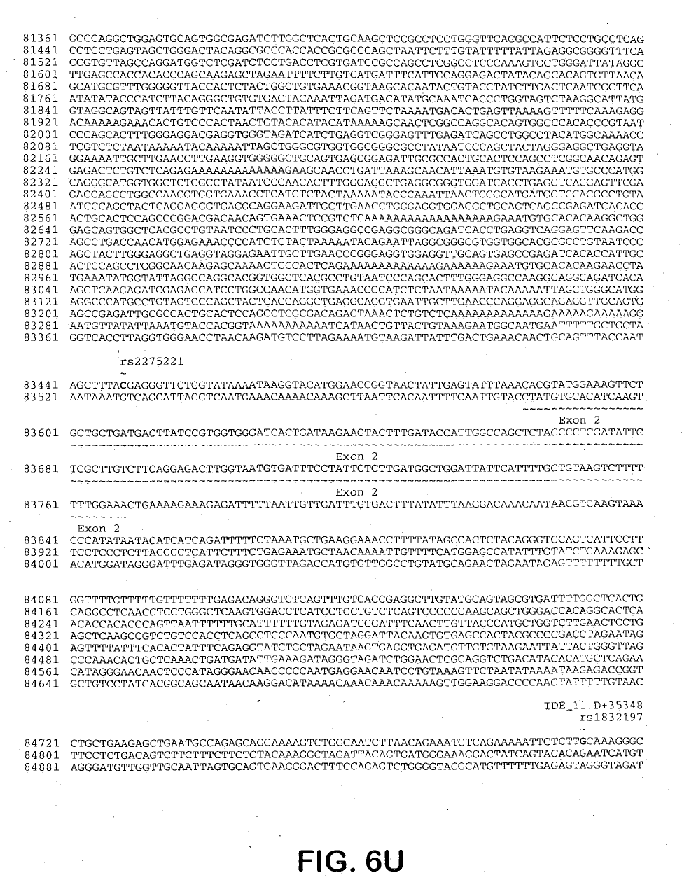

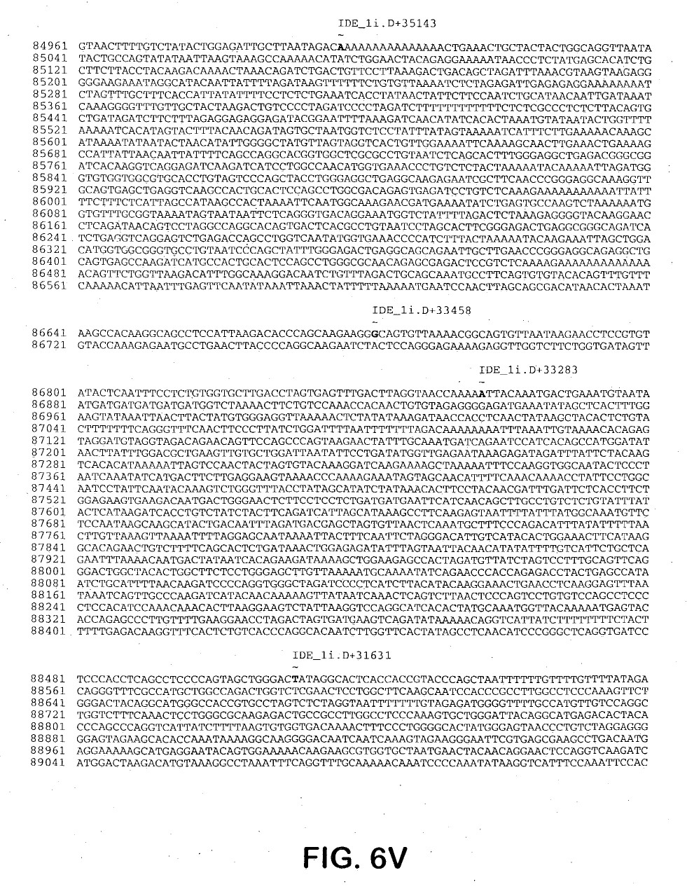

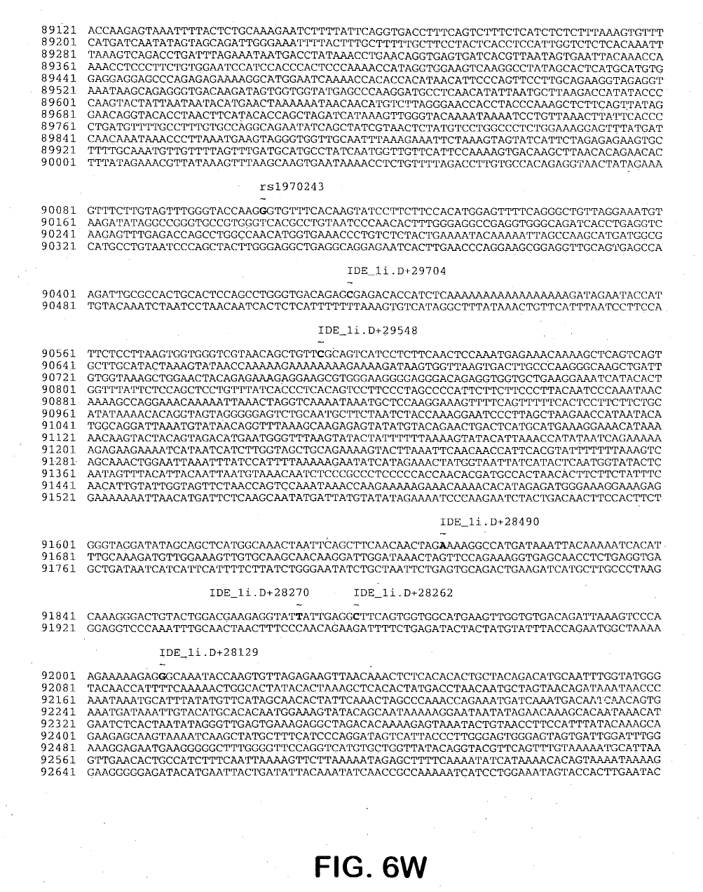

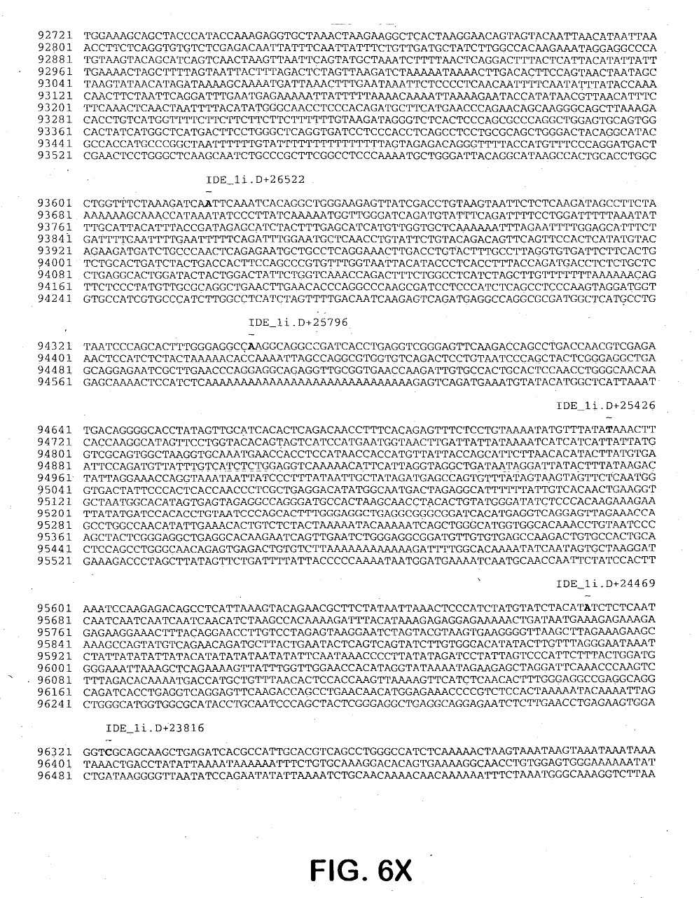

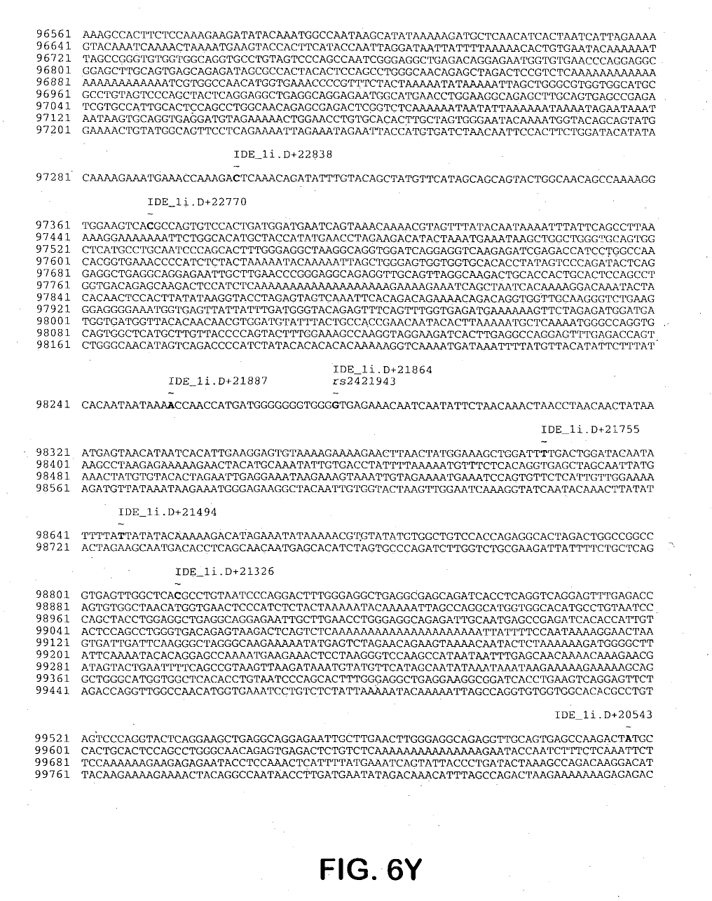

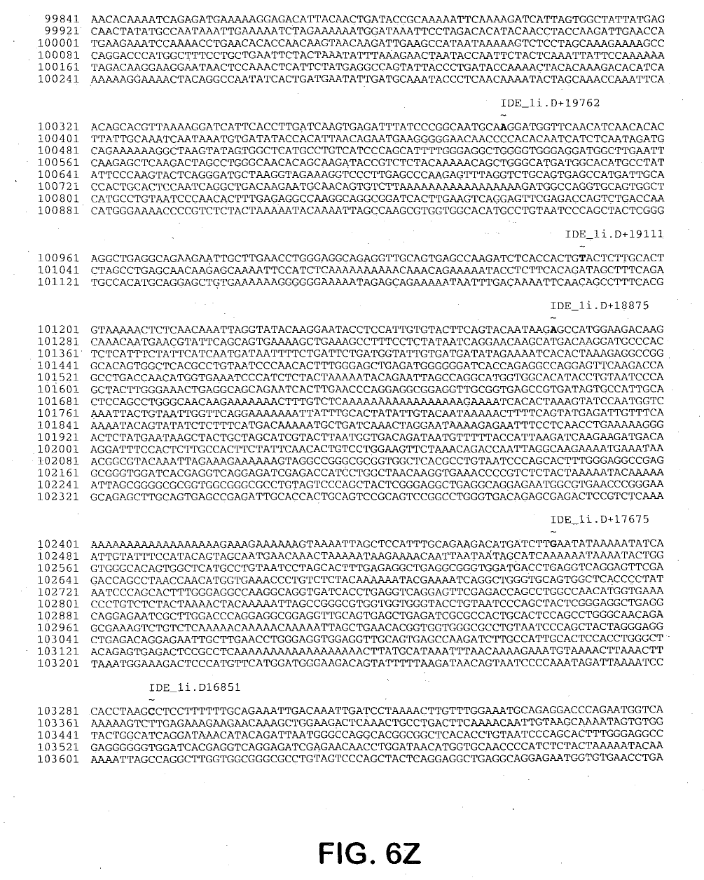

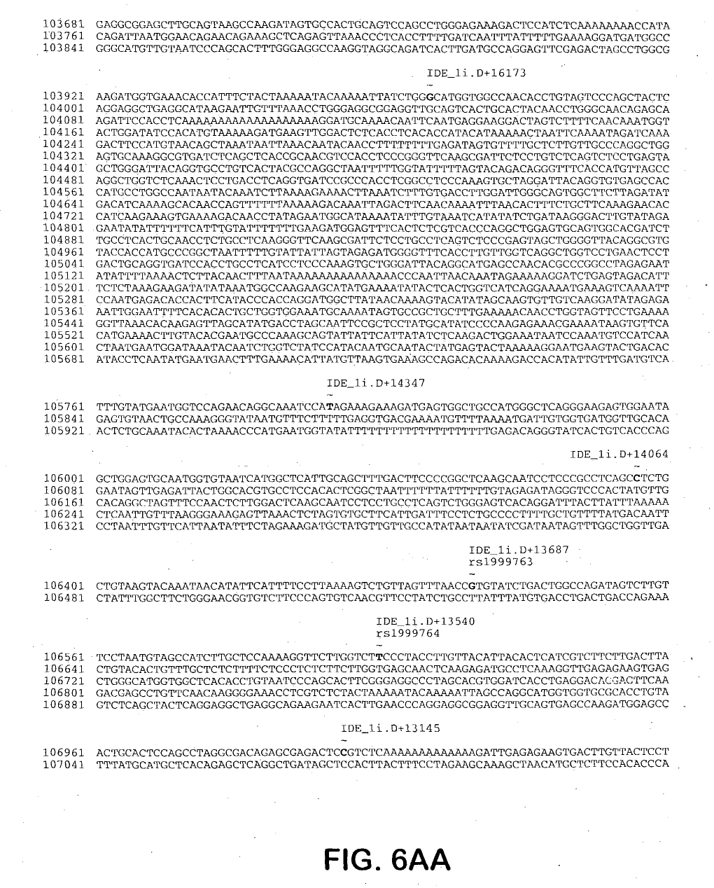

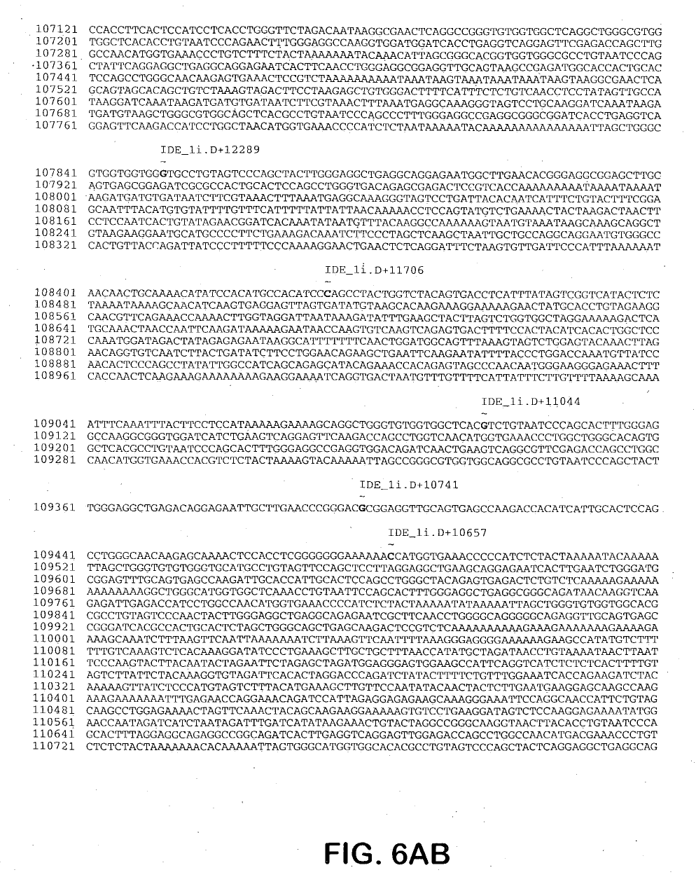

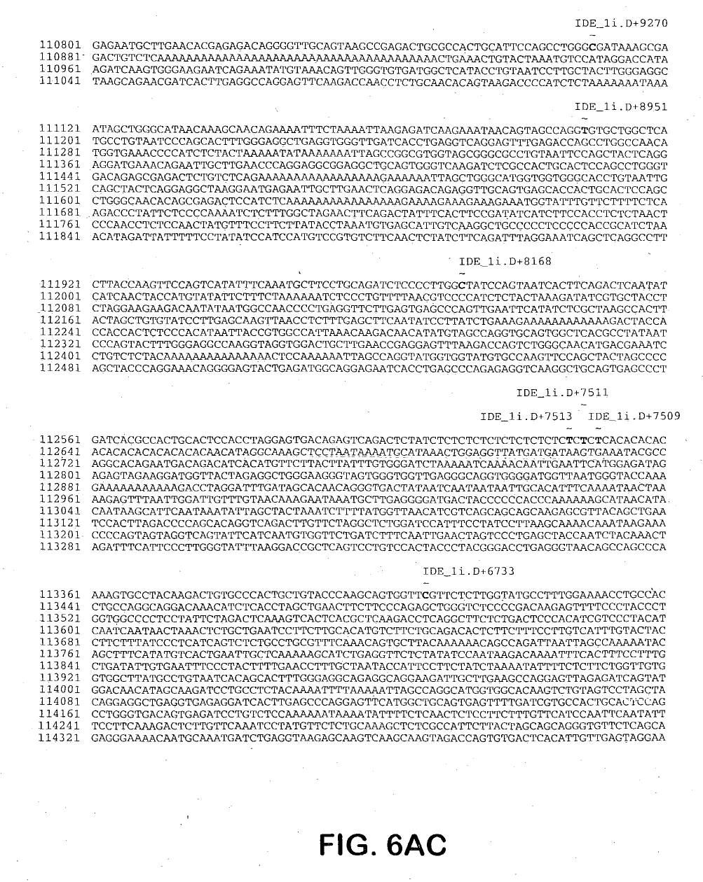

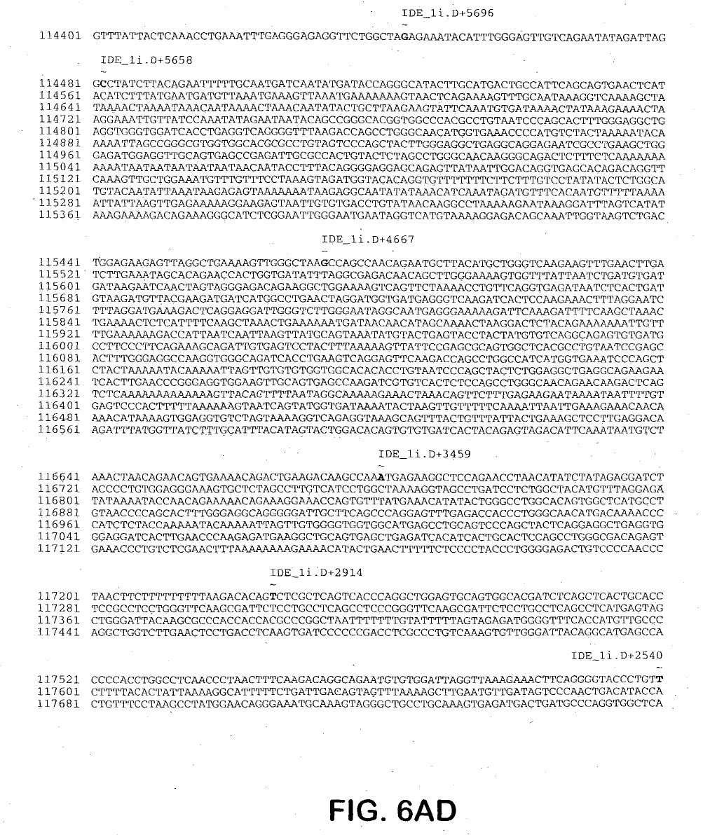

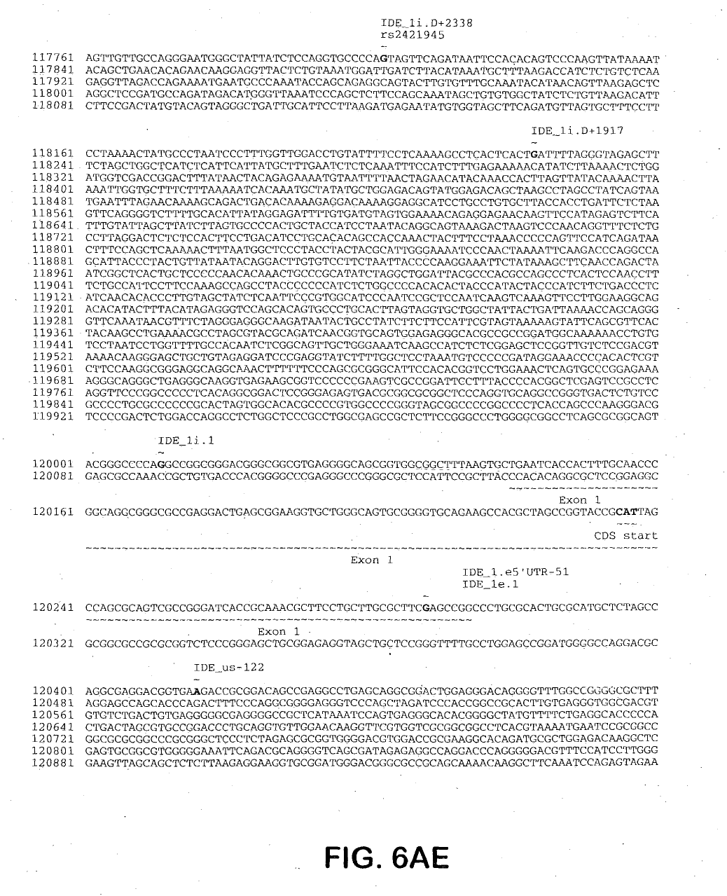

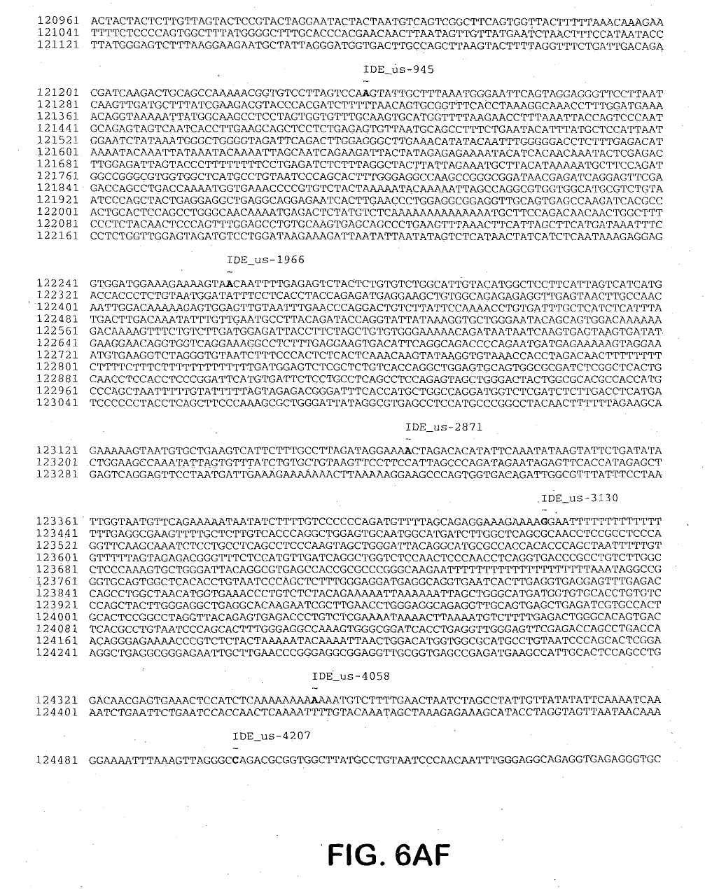

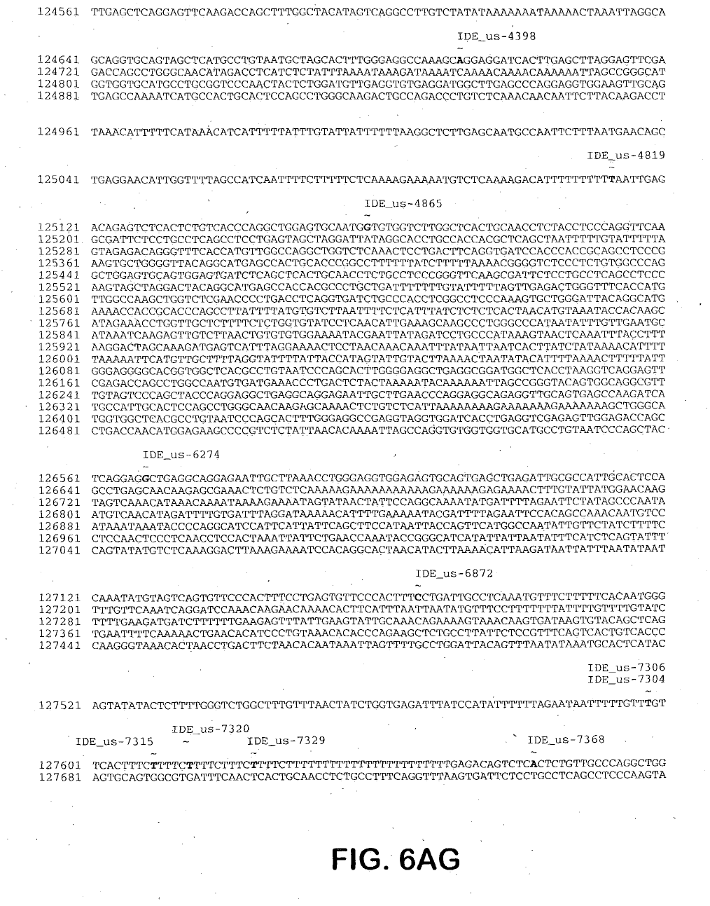

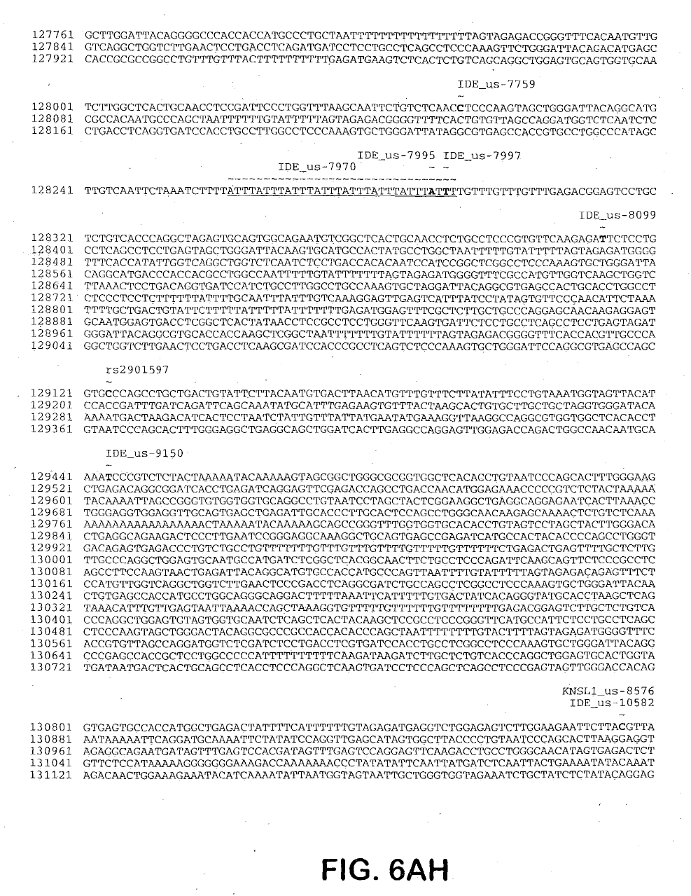

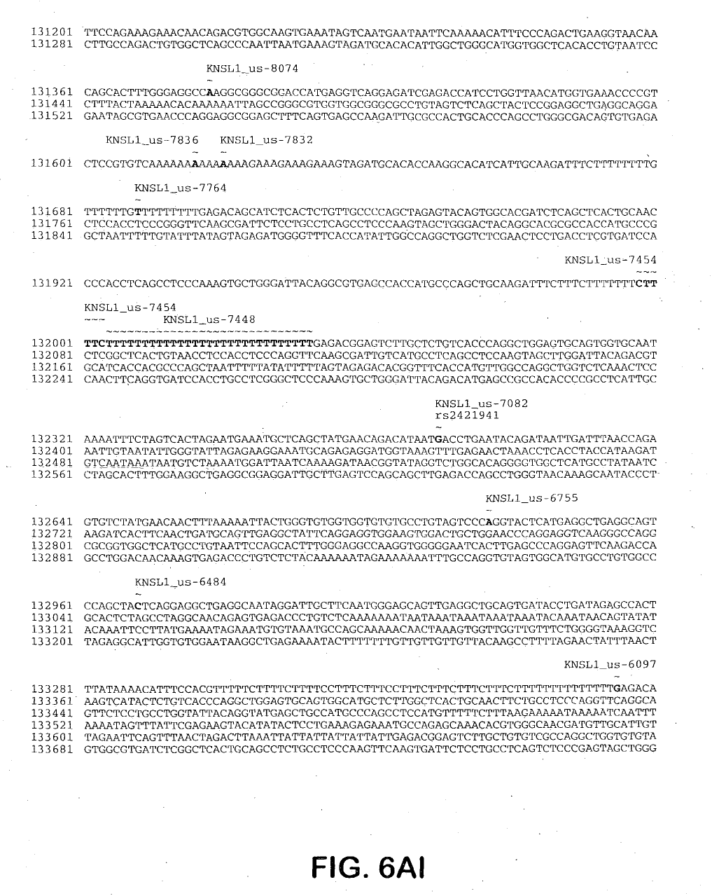

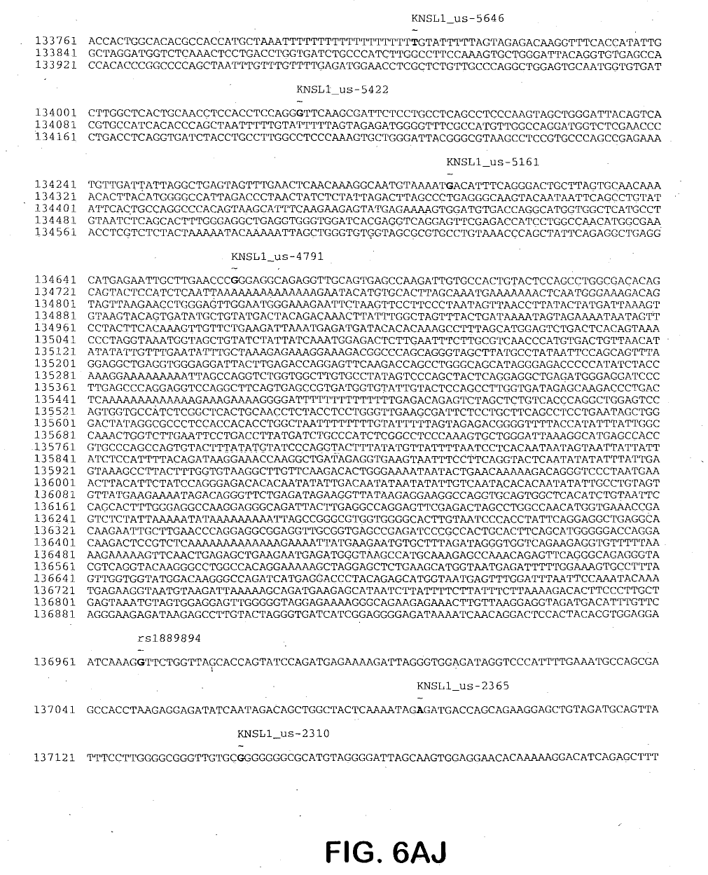

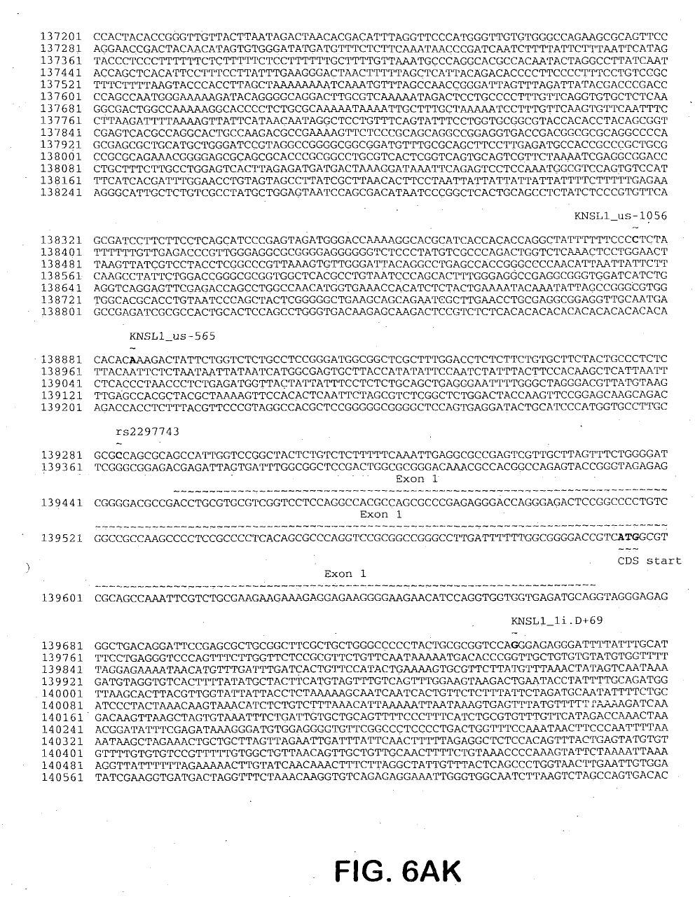

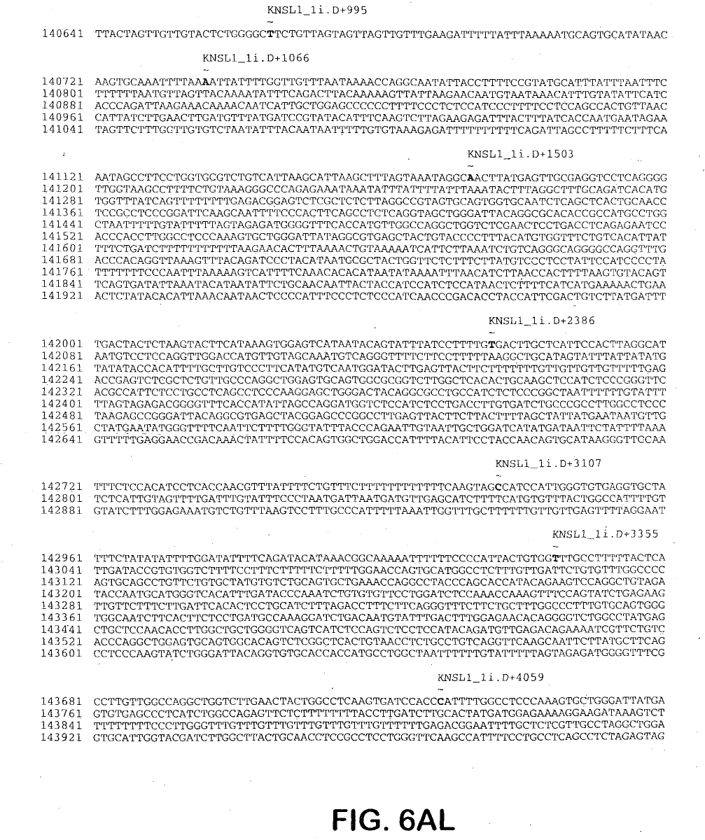

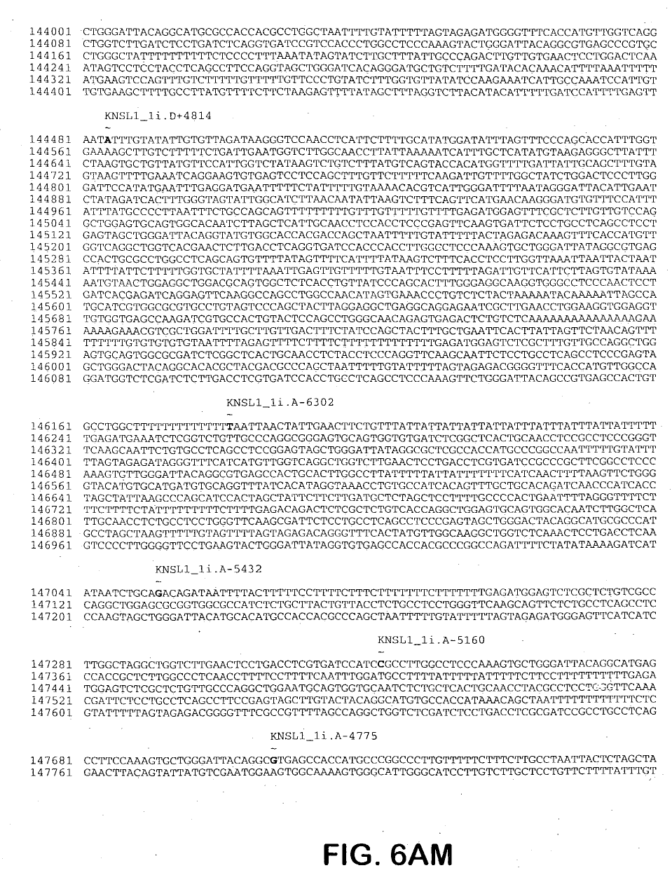

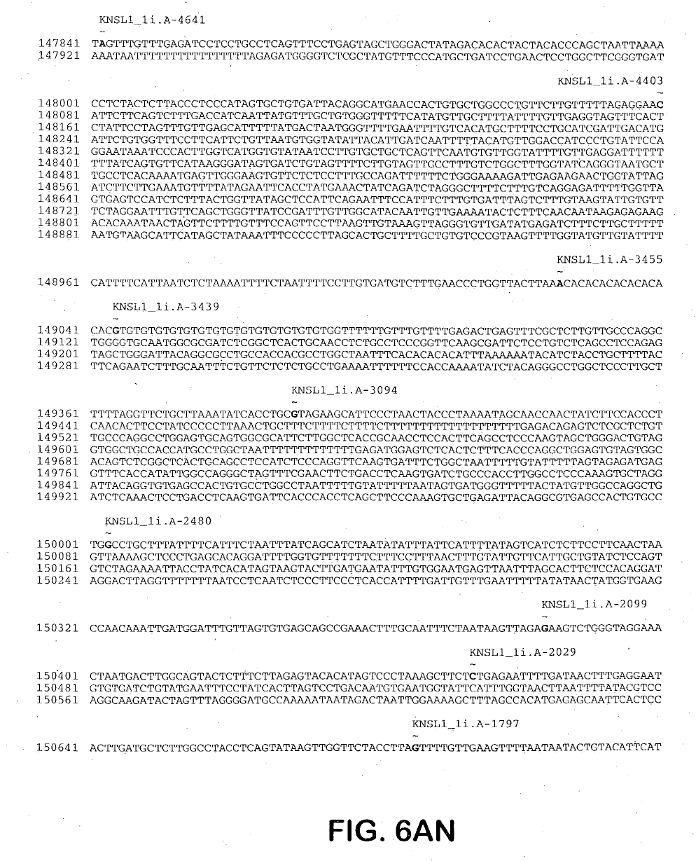

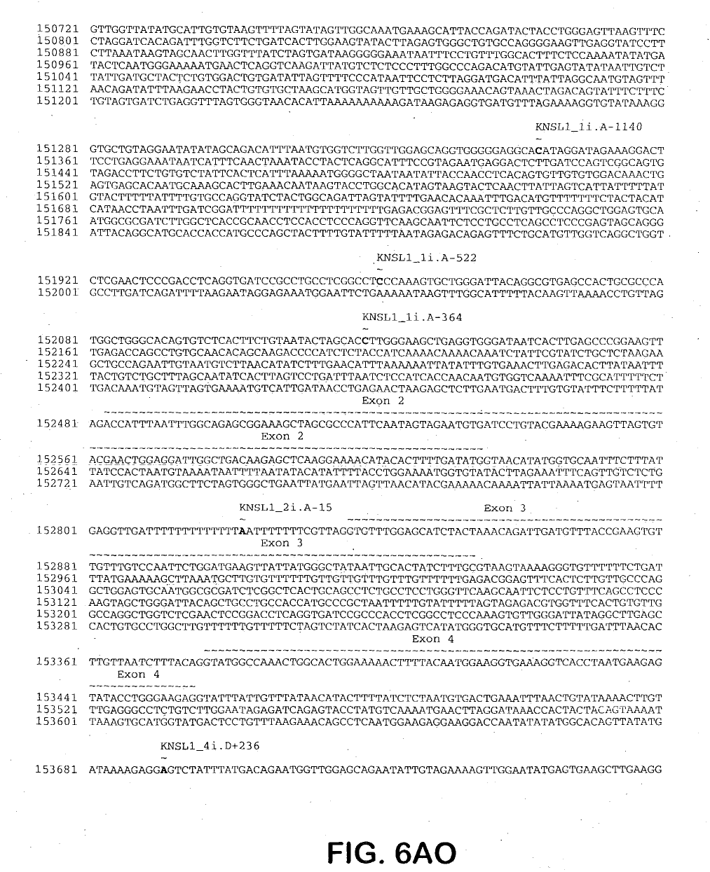

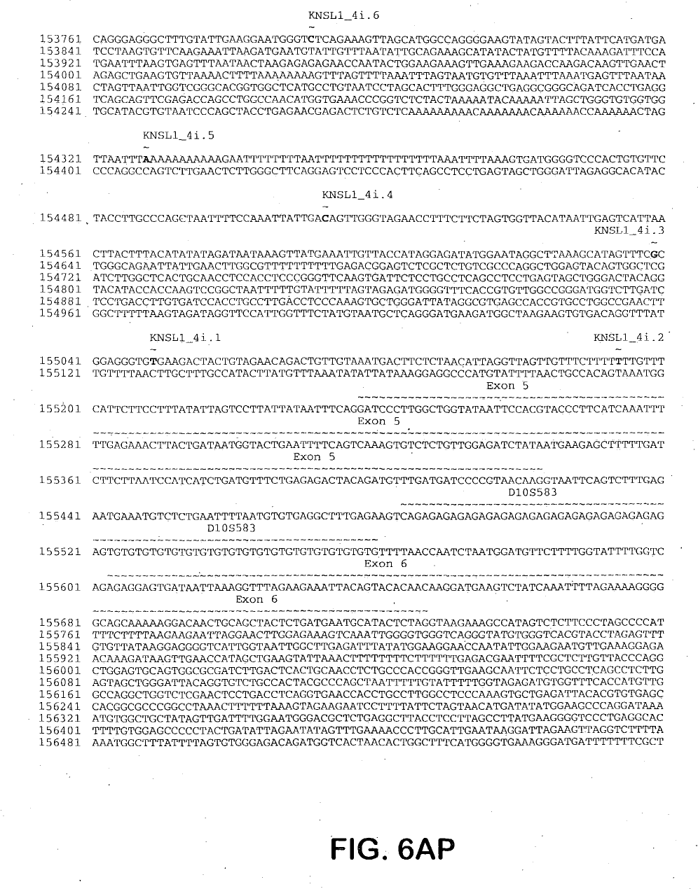

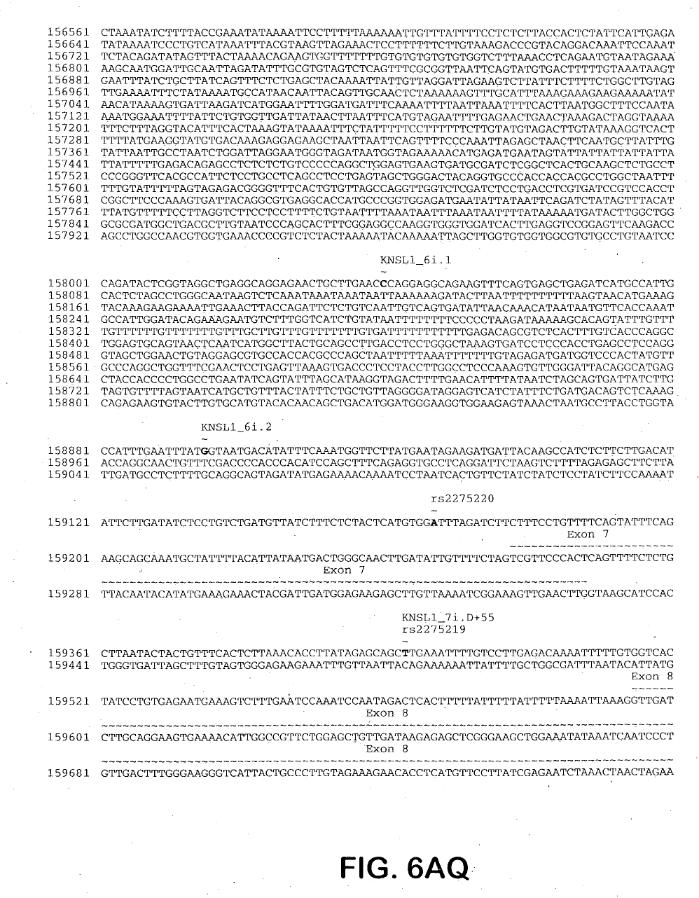

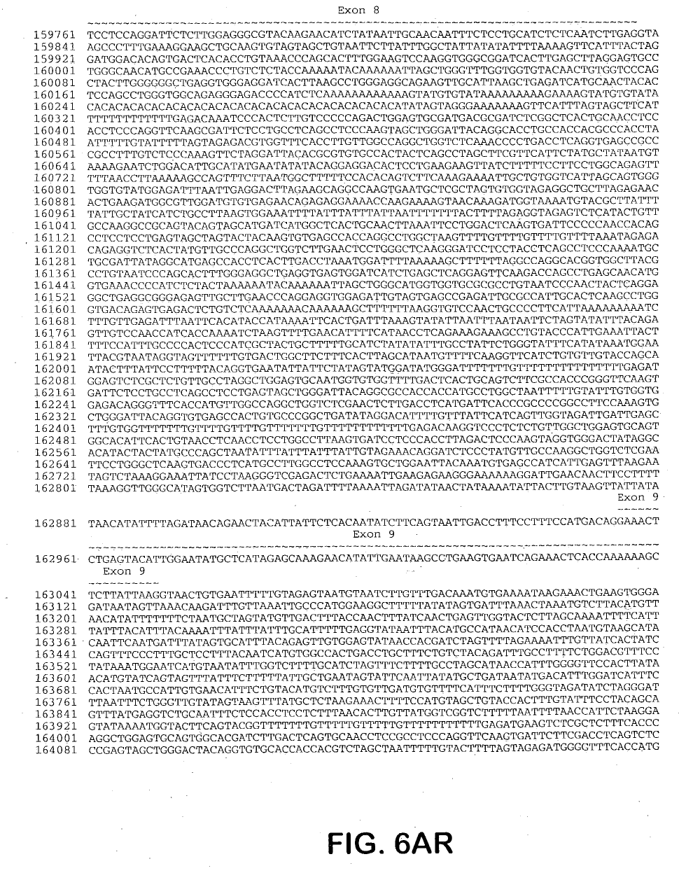

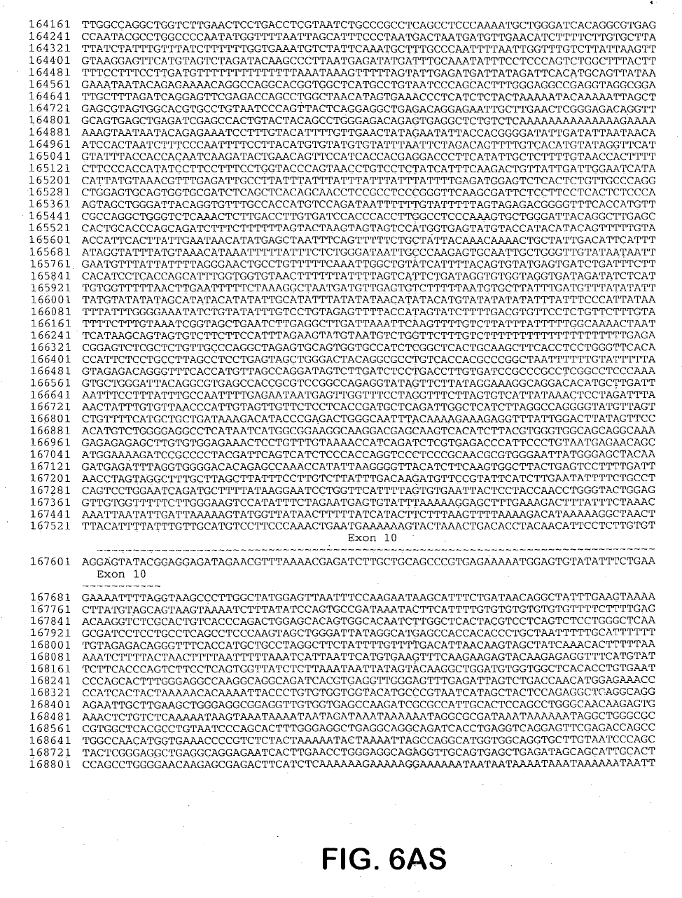

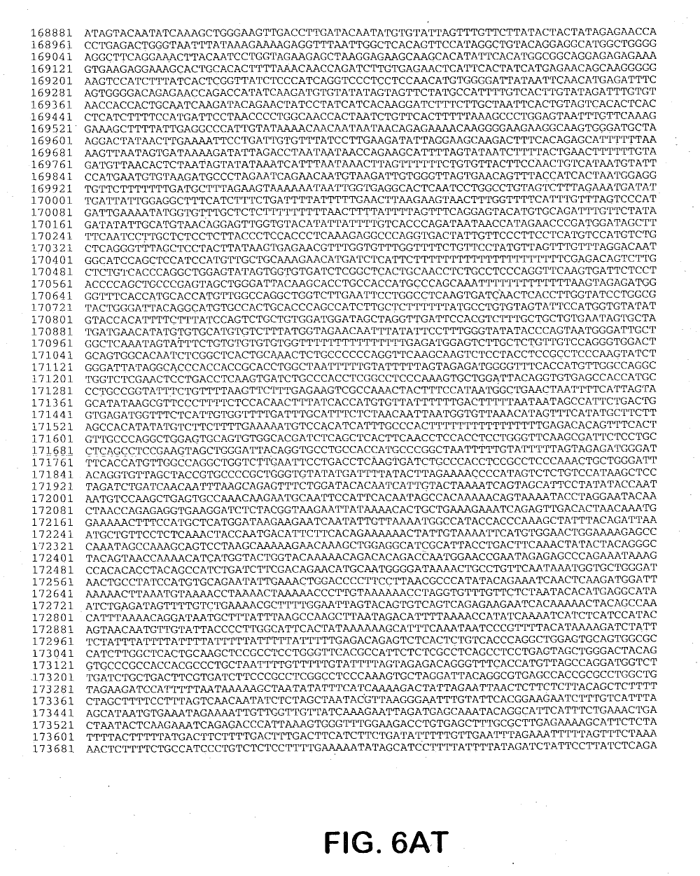

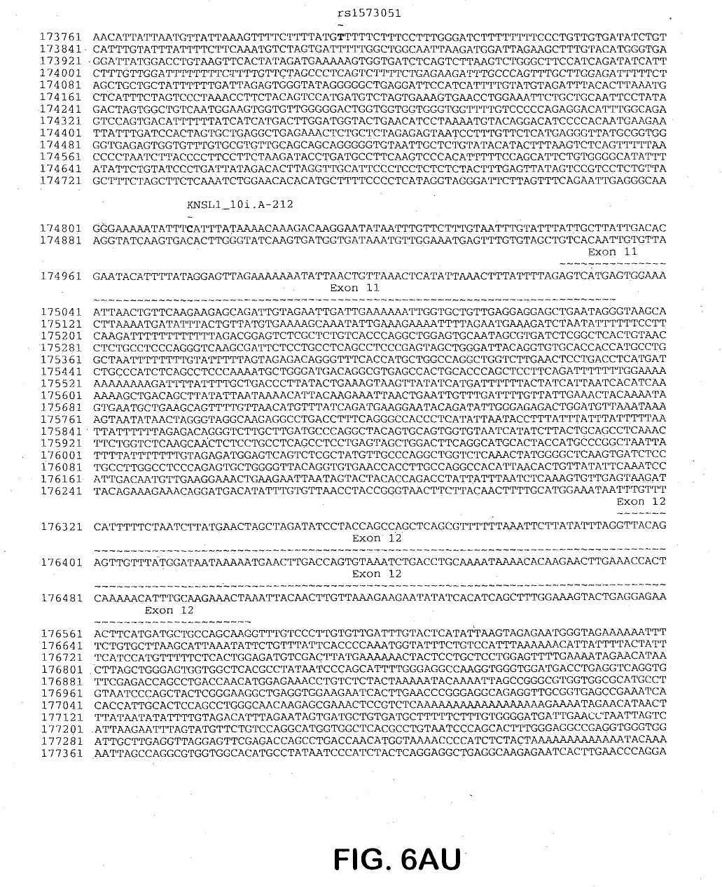

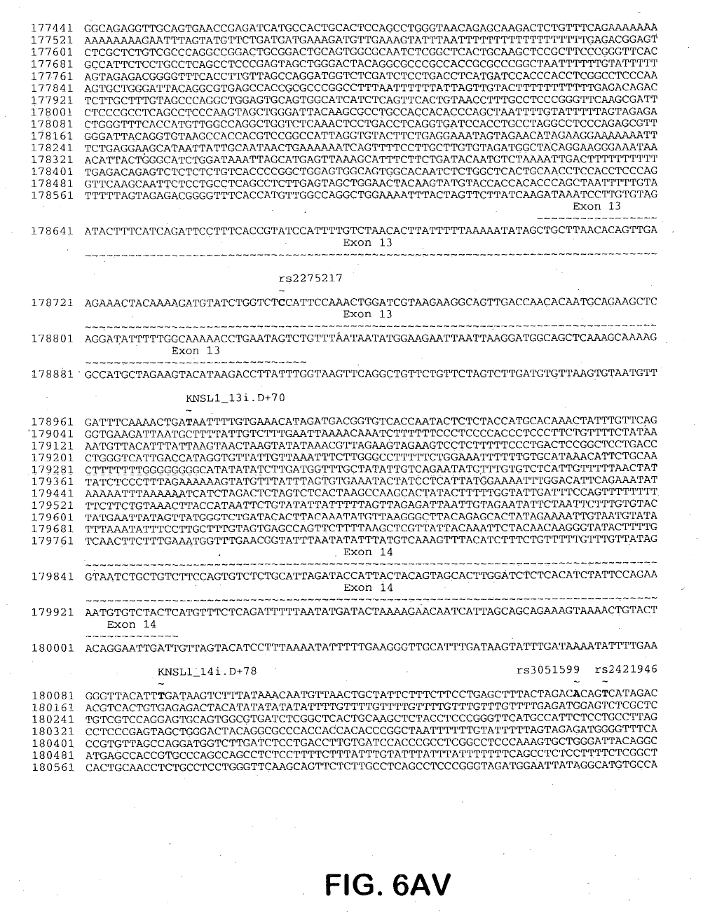

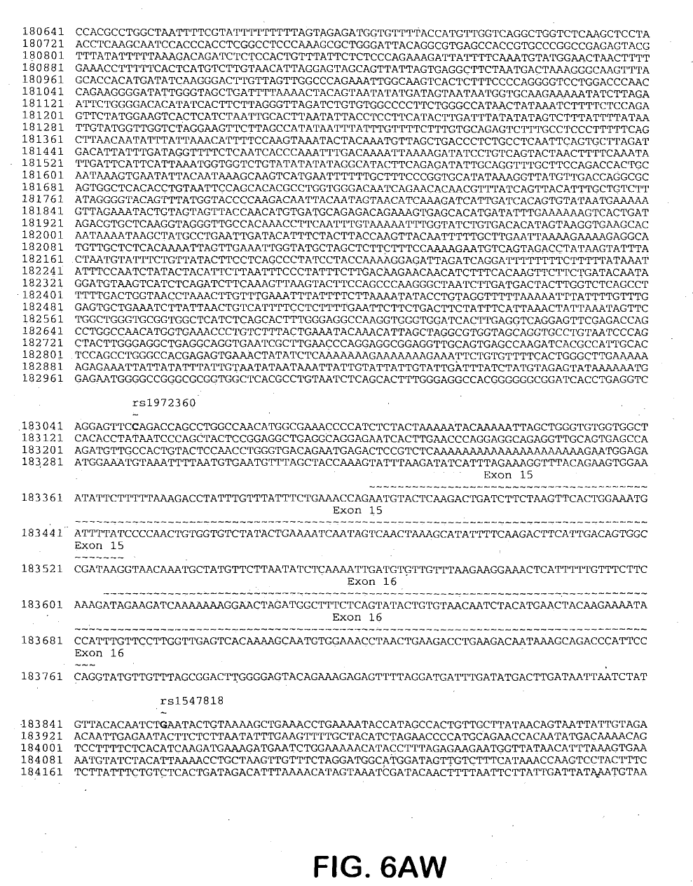

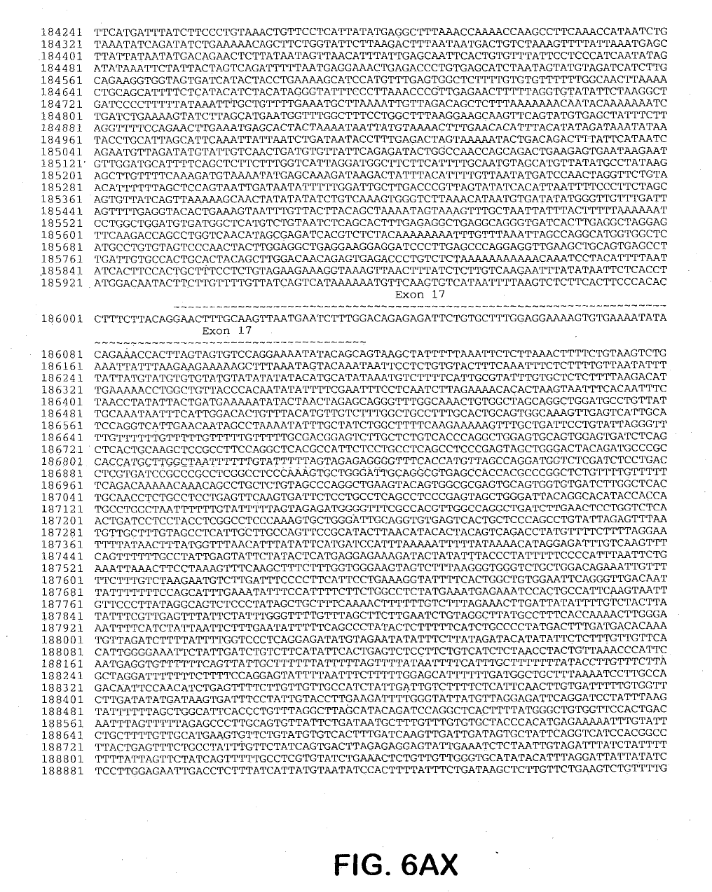

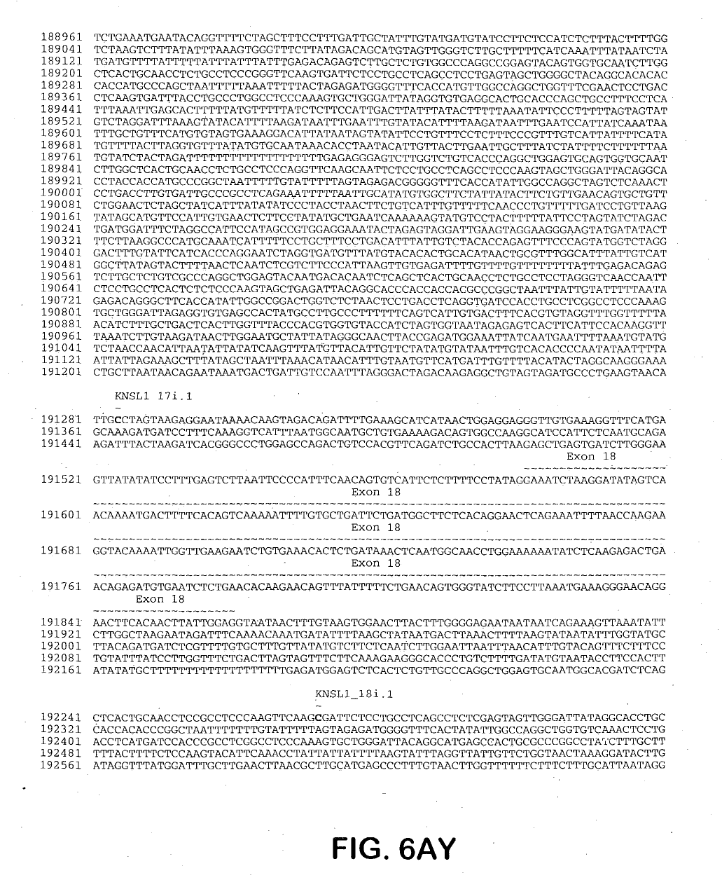

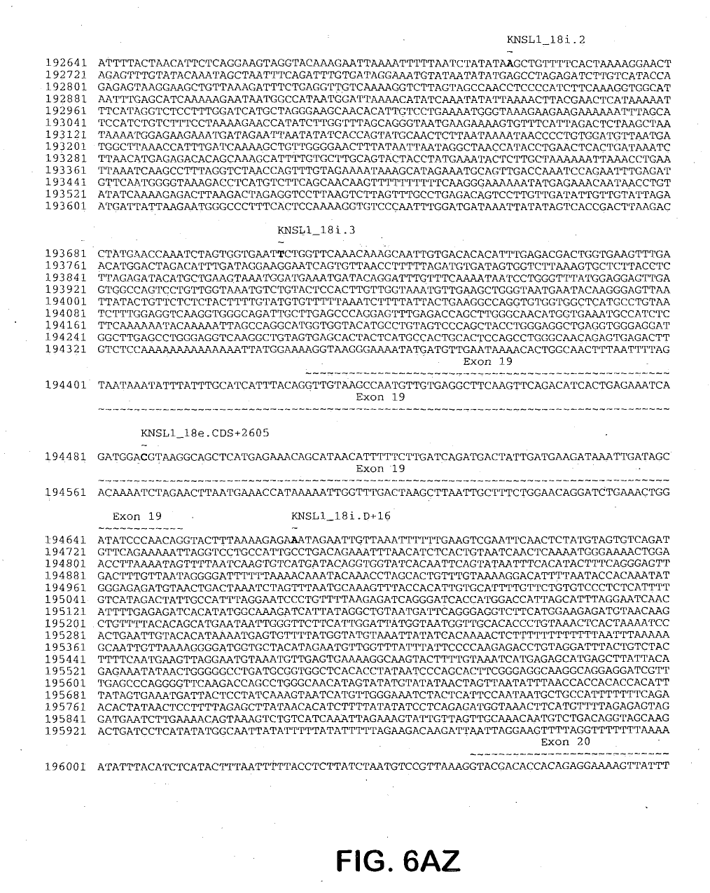

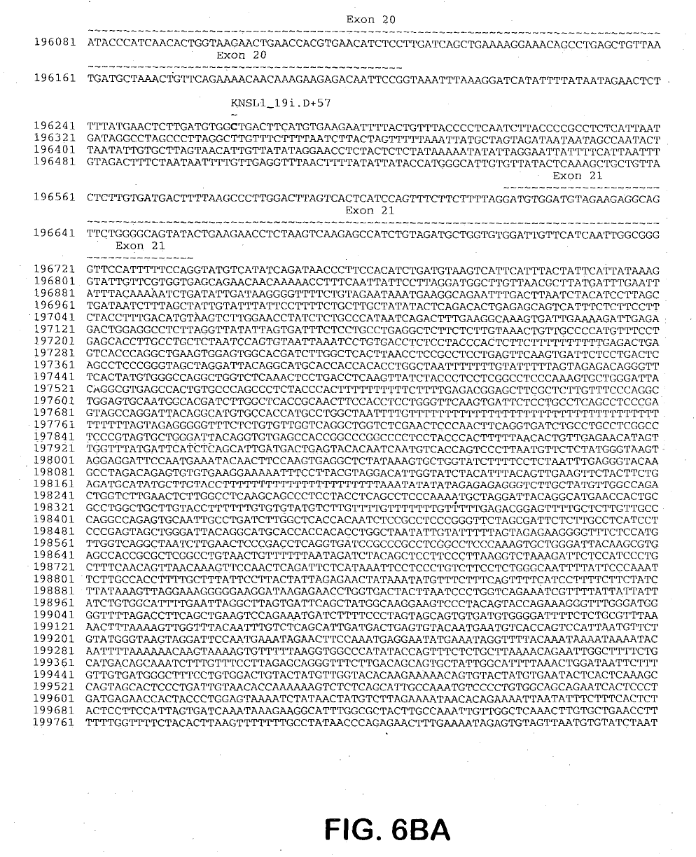

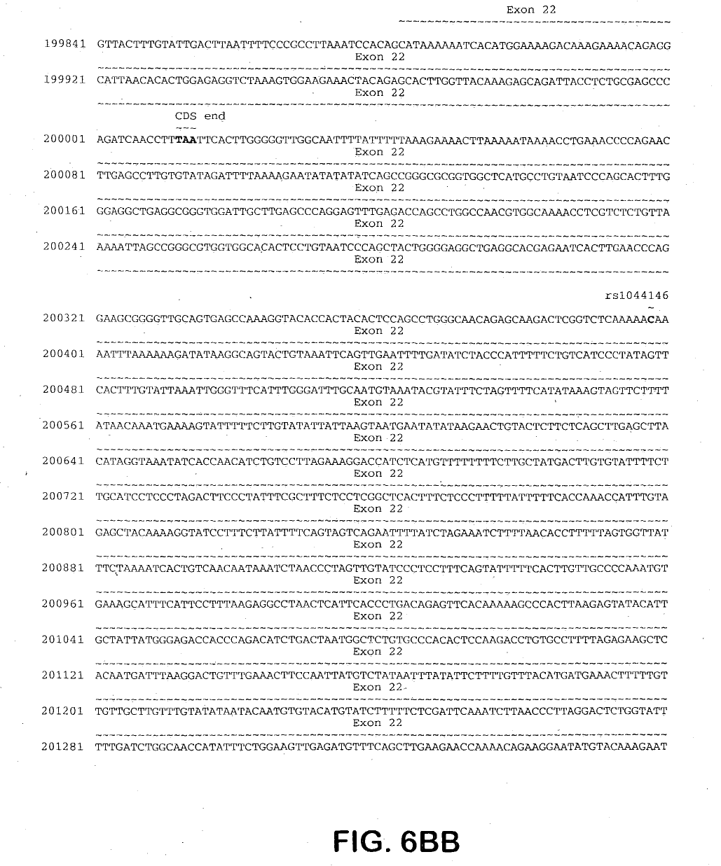

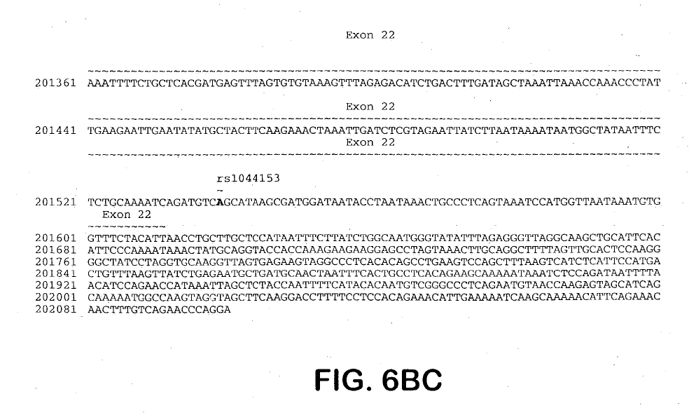

Other known SNPs in the IDE gene contemplated for use in the various diagnostic and screening methods, as well as kits and solid supports, provided herein include the NCBI SNPs set forth in Table B and Table B-2, which are referenced by their respective locations in FIG. 2 and SEQ ID NO:186; and in FIG. 6 and SEQ ID NO:484, respectively.

TABLE B
IDE NCBI Polymorphisms

| | | POSITION IN |
|---|---|---|
| NCBI SNP ID No. | POLYMORPHISM | SEQ ID NO: 186 |
| rs1999764 | A/G | 17095 |
| rs1999763 | C/T | 17242 |
| rs1970243 | C/G | 33590 |
| rs1832197 | C/T | 38903 |
| rs1855915 | A/G | 43391 |
| rs868057 | A/T | 45017 |
| rs1832195 | A/G | 68906 |
| rs1832196 | C/T | 68973 |
| rs1970245 | C/G | 73772 |
| rs1970244 | C/T | 74084 |
| rs1855916 | C/T | 83024 |
| rs1855917 | C/T | 83104 |
| rs2149632 | A/G | 105060 |
| rs544537 | A/T | 108489 |
| rs538469 | A/G | 111914 |
| rs1887922 | A/G | 113142 |
| rs1042444 | A/G | 113591 |
| rs551266 | A/G | 114683 |
| rs489517 | G/T | 117803 |
| rs568657 | A/G | 89301 |
| rs913648 | C/T | 124565 |

TABLE B-2
IDE NCBI Polymorphisms

| | POLYMORPHISM | POLYMORPHISM | |
|---|---|---|---|
| | RELATIVE TO SEQ | RELATIVE TO IDE | POSITION IN SEQ ID |
| NCBI SNP ID NO. | ID NO: 484 | SENSE STRAND | NO: 484 |
| rs568657 | C/T | G/A | 4864 |
| rs489517 | C/A | G/T | 5965 |
| rs2247348 | C/T | G/A | 6078 |
| rs520711 | A/T | T/A | 7106 |
| rs551266 | G/A | C/T | 9085 |
| rs1042444 | C/T | G/A | 10177 |
| rs1887922 | C/T | G/A | 10626 |
| rs2275218 | A/G | T/C | 11758 |
| rs538469 | A/G | T/C | 11854 |
| rs544537 | A/T | T/A | 15279 |
| rs2250090 | G/A | C/T | 18267 |
| rs2149632 | T/C | A/G | 18708 |
| rs2249960 | A/G | T/A | 19581 |
| rs2421940 | A/G | T/C | 30078 |
| rs1855917 | G/A | C/T | 40664 |
| rs1855916 | G/A | C/T | 40744 |
| rs1970244 | G/A | C/T | 49684 |
| rs1970245 | C/G | G/C | 49996 |
| rs1832196 | G/A | C/T | 54795 |
| rs1832195 | T/C | A/G | 54862 |
| rs2421942 | C/G | G/C | 73841 |
| rs868057 | A/T | T/A | 78678 |
| rs1855915 | C/T | G/A | 80304 |
| rs2275221 | C/T | G/A | 83448 |
| rs1970243 | C/G | G/C | 90105 |
| rs2421943 | A/G | T/C | 98276 |
| rs1999763 | A/G | T/C | 106453 |
| rs1999764 | C/T | G/A | 106600 |
| rs2421945 | A/G | T/C | 117802 |
| rs2901597 | T/C | A/G | 129124 |

Amplification and Genotyping IDE Haplotype Polymorphisms:

As set forth herein, an exemplary haplotype useful in the methods provided herein for determining a predisposition or occurrence of neurodegenerative disease, such as Alzheimer's disease, comprises multiple polymorphic regions of the IDE gene corresponding to nucleotides 2456, 3279, 3407 and 42943 of SEQ ID NO:187. In one embodiment, the nucleotide in IDE at position 2456 of SEQ ID NO 187 is G, at position 3279 of SEQ ID NO: 187 is T, at position 3407 of SEQ ID NO: 187 is T, and at position 42943 of SEQ ID NO:187 is T. In another embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is T. In still a further embodiment, the nucleotide in IDE at position 2456 of SEQ ID NO:187 is T, at position 3279 of SEQ ID NO:187 is T, at position 3407 of SEQ ID NO:187 is C, and at position 42943 of SEQ ID NO:187 is C. In yet another embodiment, the nucleotide in IDE at position 2456 of SEQ ID NO:187 is T, at position 3279 of SEQ ID NO:187 is C, at position 3407 of SEQ ID NO:187 is C, and at position 42943 of SEQ ID NO:187 is C.

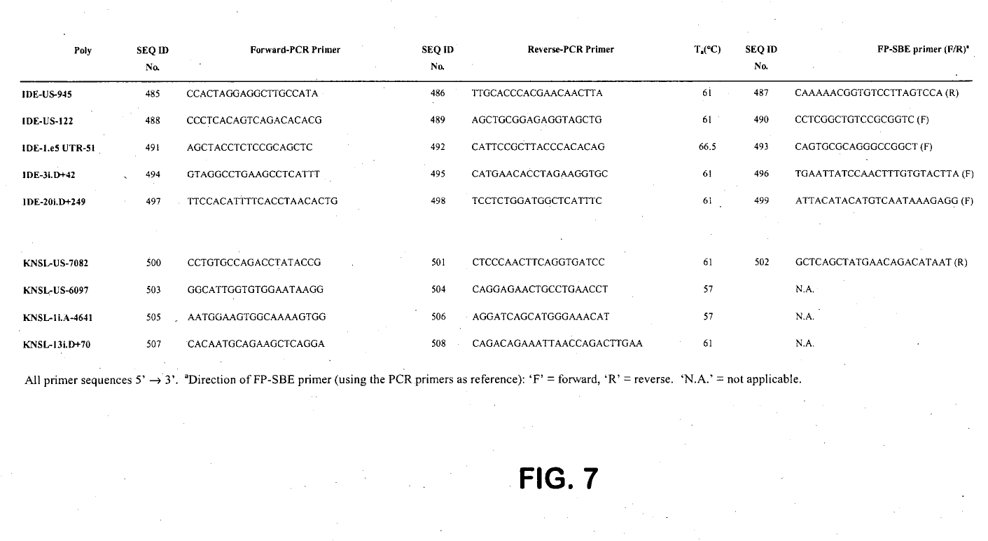

The polymorphic regions of the IDE gene corresponding to nucleotides 2456, 3279, 3407 and 42943 of SEQ ID NO:187 can be genotyped using well known methods and the PCR amplification and FP-SBE primers and conditions set forth in FIG. 7. For example, all genotypes were generated either using fluorescent polarization detected single base extension (FP-SBE, “Criterion Analyst AD”, Molecular Devices, Inc.); single base extension using capillary electrophoresis (using the “SNuPe” software on a “MegaBACE-1000” genotyping/sequencing system, Amersham-Pharmacia); or by capillary electrophoresis of PCR products using fluorescently labeled primers (using the “Genetic Profiler” software on the “MegaBACE 1000”). Generally, PCR primers were designed to yield products between 200-400 bp in length and added to ˜10 ng of genomic DNA using individually optimized PCR conditions. PCR primers and unincorporated dNTPs were degraded by the direct addition of exonuclease I (0.1-0.15 U/rxn) and shrimp alkaline phosphatase (1 U/rxn). The single base extension step was carried out using Thermosequenase (0.4 U/rxn) and the appropriate mix of R110-ddNTP, TAMRA-ddNTP (3 mM), and all four unlabeled ddNTPs (22 or 25 μM) to the Exol/SAP treated PCR product. To assess genotyping quality, 10% of the samples were randomly duplicated and called twice. Primer sequences, PCR and SBE cycling conditions are set forth in FIG. 7.

KNSL1

The nucleotide sequence of the KNSL1 gene in two sets of human genomic DNA samples was determined. The “D10-Top10” set was derived from 10 members of three families with the highest scores for AD linkage to genetic marker D10S583. The “D10-E16” set was derived from sixteen genomic DNA samples from four families showing the highest combined LOD scores for linkage with LOAD at 6 markers (D10S564, D10S583, D10S1710, D10S566, D10S1671 and D10S1741). Each set contained individuals both affected and unaffected with AD.

Twenty-one exons have been identified in the human KNSL1 gene by comparison of cDNA clones containing the KNSL1 coding sequence (CDS) with genomic sequence data. Exon-intron structure was determined, as previously described, from cDNA sequences from GenBank-Accession numbers XM_{—}005889, XM_{—}051151 and XM_{—}051152. For example, the cDNA corresponding to GenBank accession No. XM_{—}005889 contains a 5′UTR having 142 nucleotides (nt) 5′ to the translation initiation codon, the complete 3171-nucleotide coding sequence (CDS) and 1597 nucleotides of 3′UTR. Genomic DNA sequence corresponding to GenBank accession No. NT_{—}008769.1 was used for primer design.

Based on this information, primers were designed to amplify and sequence exons 1 through 21 and at least approximately 50-200 bp 5′ and 3′ to the exon boundaries indicated by the above cDNA sequences. Approximately 0.9 kb of the presumed promoter-containing region 5′ to exon 1 was also amplified and sequenced. Approximately 0.7 kb 3′ to the stop codon in exon 21 were amplified and sequenced. The primers used for amplifying KNSL1 genomic fragments corresponding to the 5′ regulatory region and the 21 exons and their corresponding sequencing primers are shown in Table 5.

TABLE 5

(Primers for KNSL1 Genomic PCR and Sequencing)

| | | SEQ | |
|---|---|---|---|
| | | ID | |
| NAME | SEQUENCE | NO: | PURPOSE |
| KNSL.Pm-p01 | ATGGTGAAACCACATCTCTACTG | 188 | PCR |
| KNSL.Pm-p02 | CCAGAAACTAAGCAACGACTC | 189 | PCR |
| KNSL.Pm-3s | ATGGTGAAACCACATCTCTACTG | 190 | se- |
| | | | quencing |
| KNSL.Pm-4s | CCAGAAACTAAGCAACGACTC | 191 | se- |
| | | | quencing |
| KNSL.Pm-5s | CAAGACTCCGTCTCTCACACAC | 192 | se- |
| | | | quencing |
| KNSL.Pm-6s | GCCGAGACGCTAGAATTGAGTG | 193 | se- |
| | | | quencing |
| KNSL.1-p01 | CGGGGCTCCAGTGAGGATAC | 194 | PCR |
| KNSL.1-p02 | ACACAGCAACCGGGTGTCAT | 195 | PCR |
| KNSL.01-3s | TTGGTCCGGCTACTCTGTCT | 196 | se- |
| | | | quencing |
| KNSL.01-4s | TGAACAGAACGCGGAGAACC | 197 | se- |
| | | | quencing |
| KNSL.01-5s | AAGCCCCTCCGCCCCTCACA | 198 | se- |
| | | | quencing |
| KNSL.01-6s | GCTGCGACGCCATGACGGTC | 199 | se- |
| | | | quencing |
| KNSL.2-p01 | AAGCTGCCAGAATTGTAATG | 200 | PCR |
| KNSL.2-p02 | GGACAAACAACACTTCGGTA | 201 | PCR |
| KNSL.02-3s | TTTCGCATTTTTCTTGACAA | 202 | se- |
| | | | quencing |
| KNSL.02-4s | ATTCATAATTCAGCCCACTA | 203 | se- |
| | | | quencing |
| KNSL.02-4sb | GCCCACTAGAAGCCATCTGA | 204 | se- |
| | | | quencing |
| KNSL.02-6s | ATTCAGAGACAACTGAAATT | 205 | se- |
| | | | quencing |
| KNSL.3-p01 | TTGTCAGATGGCTTCTAGTG | 206 | PCR |
| KNSL.3-p02 | CATGCACCCATATGACTCTT | 207 | PCR |
| KNSL.03-3s | GCTTCTAGTGGGCTGAATTA | 208 | se- |
| | | | quencing |
| KNSL.03-3sb | TGGCTTCTAGTGGGCTGAAT | 209 | se- |
| | | | quencing |
| KNSL.03-3sc | TAGTGGGCTGAATTATGAAT | 210 | se- |
| | | | quencing |
| KNSL.03-4s | TAAAAATACAAAAATTAGCG | 211 | se- |
| | | | quencing |
| KNSL.03-5s | TTATGGGCTATAATTGCACT | 212 | se- |
| | | | quencing |
| KNSL.03-6s | CGGGCATGGTGGCAGGCAGC | 213 | se- |
| | | | quencing |
| KNSL.03-8s | TAGTGCAATTATAGCCCATA | 214 | se- |
| | | | quencing |
| KNSL.4-p01 | AGTGTTGGGATTATAGGCTT | 215 | PCR |
| KNSL.4-p02 | TGGCCATGCTAACTTTC | 216 | PCR |
| KNSL.04-3s | GTTTTTTGTTTTTCTAGTCT | 217 | se- |
| | | | quencing |
| KNSL.04-4s | TTCAATACAAAGCCCTCCCT | 218 | se- |
| | | | quencing |
| KNSL.04-5s | TTTGAGGGCCTCTGTGTTGG | 219 | se- |
| | | | quencing |
| KNSL.04-6s | GAGGCCCTCAAACAAGTTTT | 220 | se- |
| | | | quencing |
| KNSL.5-p01 | GGGATGAAGATGGCTAAGAA | 221 | PCR |
| KNSL.5-p02 | TGACCCCTCCTTATAACA | 222 | PCR |
| KNSL.05-3s | TTGCTTTGCCATACTTATGT | 223 | se- |
| | | | quencing |
| KNSL.05-4s | ACCCCTCCTTATAACACAAA | 224 | se- |
| | | | quencing |
| KNSL.05-4sb | ATACCCTGACCCACCCCAAT | 225 | se- |
| | | | quencing |
| KNSL.05-5s | ATGATCCCCGTAACAAGGTA | 226 | se- |
| | | | quencing |
| KNSL.05-5sb | GCTTTGAGAAGTCAGAGAGA | 227 | se- |
| | | | quencing |
| KNSL.05-6s | TTTTGCTGCCCCCTTTTCTA | 228 | se- |
| | | | quencing |
| KNSL.05-7s | ATTTTAGAAAAGGGGGCAGC | 229 | se- |
| | | | quencing |
| KNSL.05-8s | CGGGGATCATCAAACATCTG | 230 | se- |
| | | | quencing |
| KNSL.6-p01 | TTGACATACCAGGCAACTGT | 231 | PCR |
| KNSL.6-p02 | GCCCACCTTGGACTTC | 232 | PCR |
| KNSL.06-3s | GTTTCGACCCCACCCACATC | 233 | se- |
| | | | quencing |
| KNSL.06-4s | ATCCGCCCACCTTGGACTTC | 234 | se- |
| | | | quencing |
| KNSL.06-4sb | CTGGGTTTACAGGTGTGAGT | 235 | se- |
| | | | quencing |
| KNSL.06-5s | TAATGACTGGGCAACTTGAT | 236 | se- |
| | | | quencing |
| KNSL.06-6s | CTACAAGGGCAGTAATGACC | 237 | se- |
| | | | quencing |
| KNSL.06-7s | TGCTGGCGATTTAATACATT | 238 | se- |
| | | | quencing |
| KNSL.06-8s | TTTCCGATTTTAACAAGCTC | 239 | se- |
| | | | quencing |
| KNSL.7-p01 | ATTATCCTAAGGGTCGAGAC | 240 | PCR |
| KNSL.7-p02 | TGGATGTTATGGCATGTAAA | 241 | PCR |
| KNSL.07-3s | AAAGGTTGGGCATAGTGGTC | 242 | se- |
| | | | quencing |
| KNSL.07-4s | ATGGCATGTAAATTATACCT | 243 | se- |
| | | | quencing |
| KNSL.07-5s | TGAGTACATTGGAATATGCT | 244 | se- |
| | | | quencing |
| KNSL.07-6s | CCTTCCATGGGCAATTTAAC | 245 | se- |
| | | | quencing |
| KNSL.8-p01 | ACAAGATGTTCCGTATTCAT | 246 | PCR |
| KNSL.8-p02 | GGTGACAGTGCGAGACCT | 247 | PCR |
| KNSL.08-3s | GTGGTTTTTCTTGGGAAGTC | 248 | se- |
| | | | quencing |
| KNSL.08-4s | TGGGCACTGGATATAAAGAT | 249 | se- |
| | | | quencing |
| KNSL.08-5s | TGTCCTTCCCAAACTGAATG | 250 | se- |
| | | | quencing |
| KNSL.9-p01 | CCGTCCTCTGTTAGCTTTC | 251 | PCR |
| KNSL.9-p02 | CTCTTGCCTACCCTAGTTAT | 252 | PCR |
| KNSL.09-3s | CACACATGCTTTTCCCCTCA | 253 | se- |
| | | | quencing |
| KNSL.09-4s | CCCAATATCTGTATTCCTTC | 254 | se- |
| | | | quencing |
| KNSL.09-5s | AATGTTGGAAATGAGTTTGT | 255 | se- |
| | | | quencing |
| KNSL.09-6s | GCTGAGATGGGCAGATCATG | 256 | se- |
| | | | quencing |
| KNSL.09-6sb | GAAGGAAAAAATATTAGATC | 257 | se- |
| | | | quencing |
| KNSL.09-7s | ATTTTTTCCTTCAAGATTTT | 258 | se- |
| | | | quencing |
| KNSL.09-8s | CTTACCCTATTCAGCTCCTC | 259 | se- |
| | | | quencing |
| KNSL.10-p01 | CCAGGCCACATTAACAC | 260 | PCR |
| KNSL.10-p02 | CCAGGAGCAGGAGTAGTTTT | 261 | PCR |
| KNSL.10-3s | GTTAACCTACCGGGTAACTT | 262 | se- |
| | | | quencing |
| KNSL.10-4s | GCTGGCAGCATCATGAAGTT | 263 | se- |
| | | | quencing |
| KNSL.10-4sb | AGTGAGAAAAACATGGATGA | 264 | se- |
| | | | quencing |
| KNSL.10-5s | TGTAAATCTGACCTGCAAAA | 265 | se- |
| | | | quencing |
| KNSL.10-6s | AAGAAGTTACCCGGTAGGTT | 266 | se- |
| | | | quencing |
| KNSL.11-p01 | GTGGCAGTGGCACAATCTCT | 267 | PCR |
| KNSL.11-p02 | GCCCAAGAAATTTAACAAT | 268 | PCR |
| KNSL.11-3s | TGGGCAGGCTGGAAAATTTA | 269 | se- |
| | | | quencing |
| KNSL.11-4s | AGGGTCAGGAGCCGGAGTCA | 270 | se- |
| | | | quencing |
| KNSL.11-4sb | TTGTGCATGGTAGAGAGTAT | 271 | se- |
| | | | quencing |
| KNSL.11-5s | AGGCCATGCTAGAAGTACAT | 272 | se- |
| | | | quencing |
| KNSL.11-6s | GGCCTTTTGCTTTGAGCTGC | 273 | se- |
| | | | quencing |
| KNSL.12-p01 | CACTAAGCCAAGCACTATAC | 274 | PCR |
| KNSL.12-p02 | CTGCACTCCTGGACGA | 275 | PCR |
| KNSL.12-3s | AATATGTTAAGGGCTTACAG | 276 | se- |
| | | | quencing |
| KNSL.12-4s | TGTAGTCTCTCACAGTGACG | 277 | se- |
| | | | quencing |
| KNSL.12-5s | AGCACTTGGATCTCTCACAT | 278 | se- |
| | | | quencing |
| KNSL.12-6s | GTAGACACATTTTCTGGAAT | 279 | se- |
| | | | quencing |
| KNSL.13-p01 | ATGTTGCCACTGTACTCCAA | 280 | PCR |
| KNSL.13-p02 | TATCCATGCCATCCTAGAA | 281 | PCR |
| KNSL.13-3s | ATGTTGCCACTGTACTCCAA | 282 | se- |
| | | | quencing |
| KNSL.13-3sb | AAAGAATGGAGAATGGAAAT | 283 | se- |
| | | | quencing |
| KNSL.13-3sc | TGTGAATGTTTAGCTACCAA | 284 | se- |
| | | | quencing |
| KNSL.13-4s | TGTGCATGGGGTTCTAGATG | 285 | se- |
| | | | quencing |
| KNSL.13-5s | TATCCCCAACTGTGGTGTCT | 286 | se- |
| | | | quencing |
| KNSL.13-6s | CCCAAGTCCGCTAAACAACA | 287 | PCR |
| KNSL.13-7s | AAGAAAATACCATTTGTTCC | 288 | PCR |
| KNSL.13-8s | CGGCCACTGTCAATGAAGTC | 289 | se- |
| | | | quencing |
| KNSL.14-p01 | CATAGCGAGATCACGTCTCT | 290 | se- |
| | | | quencing |
| KNSL.14-p02 | GCCAAACCCTGCTCTAGTT | 291 | se- |
| | | | quencing |
| KNSL.14-3s | AGCCCAGGAGGTTGAAGCTG | 292 | se- |
| | | | quencing |
| KNSL.14-3sb | TTCTCACCTATGGACAATAC | 293 | se- |
| | | | quencing |
| KNSL.14-4s | AGAGCACAAATACGCAATGA | 294 | se- |
| | | | quencing |
| KNSL.14-4sb | TACACAGAGGAATTATTTGT | 295 | se- |
| | | | quencing |
| KNSL.14-5s | AGTCTCTTCACTTCCCACAC | 296 | se- |
| | | | quencing |
| KNSL.14-6s | TTCCTCCAAAGCACAGAATC | 297 | se- |
| | | | quencing |
| KNSL.f15 p01n | GAAGATTTACTAAGATCACGG | 298 | PCR |
| KNSL.f15 p02n | TTTTGAAATCTATTCTTAGCCA | 299 | PCR |
| KNSL.15-3s | TAACTCCTTTTAGAGCTTAT | 300 | se- |
| | | | quencing |
| KNSL.15-3sb | GAAGATTTACTAAGATCACGG | 301 | se- |
| | | | quencing |
| KNSL.15-4s | AATGAGAGGCGGGGTAAGAT | 302 | se- |
| | | | quencing |
| KNSL.15-4sb | TTTTGAAATCTATTCTTAGCCA | 303 | se- |
| | | | quencing |
| KNSL.15-5s | CTCTTATCTAATGTCCGTTA | 304 | se- |
| | | | quencing |
| KNSL.15-6s | AACGGACATTAGATAAGAGG | 305 | se- |
| | | | quencing |
| KNSL.16-p01 | TGTAGTCCCAGCTACCTG | 306 | PCR |
| KNSL.16-p02 | ATGGCAATAGTCTATGACAA | 307 | PCR |
| KNSL.16-3s | GGGCAACAGAGTGAGACTTG | 308 | se- |
| | | | quencing |
| KNSL.16-3sb | TTATGGAAAAGGTAAGGGAAAA | 309 | se- |
| | | | quencing |
| KNSL.16-4s | GCAATGGCAGGACCTAATTT | 310 | se- |
| | | | quencing |
| KNSL.16-5s | TCATAAATTCCTCCCTGTCT | 311 | se- |
| | | | quencing |
| KNSL.16-6s | AAAACAGTTACAGGCCGAGC | 312 | se- |
| | | | quencing |
| KNSL.17-p01 | CAGCCTGGGCAACATAGTAT | 313 | PCR |
| KNSL.17-p02 | CTAAGTCCAAGGGCTTAAA | 314 | PCR |
| KNSL.17-3s | GCCTGGGCAACATAGTATAT | 315 | se- |
| | | | quencing |
| KNSL.17-4s | ATTAATGAGAGGCGGGGTAA | 316 | se- |
| | | | quencing |
| KNSL.17-5s | TTAGTTGCAAACAATGTCTG | 317 | se- |
| | | | quencing |
| KNSL.17-6s | TACCTTTAACGGACATTAGA | 318 | se- |
| | | | quencing |
| KNSL.17-6sb | AATGAGAGGCGGGGTAAGATTG | 319 | se- |
| | | | quencing |
| KNSL.17-7s | GAAAAGGAAACAGCCTGAGC | 320 | se- |
| | | | quencing |
| KNSL.17-8s | TTACCATCTCTGAGGATATA | 321 | se- |
| | | | quencing |
| KNSL.18-p01 | AGAGACAATTCCGGTAAAT | 322 | PCR |
| KNSL.18-p02 | TTATGGGCAGAGATAGGTTC | 323 | PCR |
| KNSL.18-3s | TCTTACCCCGCCTCTCATTA | 324 | se- |
| | | | quencing |
| KNSL.18-4s | CCCCTTATCAATATCAGATT | 325 | se- |
| | | | quencing |
| KNSL.18-5s | TGGGCATTGGGTTATACTCA | 326 | se- |
| | | | quencing |
| KNSL.18-6s | AGTATACTGCCCCAGAACTG | 327 | se- |
| | | | quencing |
| KNSL.19-p01n | TGATGAGAACCACTACCCTG | 328 | PCR |
| KNSL.19-p02n | TTTAACAGAGACGAGGTTTT | 329 | PCR |
| KNSL.19-3s | AAGAAGGCATTTGGCGCTAC | 330 | se- |
| | | | quencing |
| KNSL.19-3sb | TTTGCCTATAACCCAGAGAACT | 331 | se- |
| | | | quencing |
| KNSL.19-4s | AAGTGAGCCGAGGAGAAAGC | 332 | se- |
| | | | quencing |
| KNSL.19-5s | ACTTGGTTACAAAGAGCAGA | 333 | se- |
| | | | quencing |
| KNSL.19-5sb | CTGCGAGCCCAGATCAACCTTT | 334 | se- |
| | | | quencing |
| KNSL.19-6s | ATGAAACCCAATTTAATACA | 335 | se- |
| | | | quencing |
| KNSL.19-6sb | AATCTATACACAAGGCTCAAGT | 336 | se- |
| | | | quencing |
| KNSL.19-7s | GTGGCAAAACCTCGTCTCTG | 337 | se- |
| | | | quencing |
| KNSL.19-8s | GCCCGGCTAATTTTTAACAG | 338 | se- |
| | | | quencing |
| KNSL.19-9s | GGCAACAGAGCAAGACTCGG | 339 | se- |
| | | | quencing |
| KNSL.19-10s | CTGGGCTCGCAGAGGTAATC | 340 | se- |
| | | | quencing |
| KNSL.20-p01n | CGAGCCCAGATCAACCTT | 341 | PCR |
| KNSL.20-p02n | CCGAGGAGAAAGCGAAATAG | 342 | PCR |
| KNSL.20-3s | GGAGCCCAGATCAACCTT | 343 | se- |
| | | | quencing |
| KNSL.20-4s | CCGAGGAGAAAGCGAAATAG | 344 | se- |
| | | | quencing |
| KNSL.20-5s | CGTGGCAAAACCTCGTCTCTGT | 345 | se- |
| | | | quencing |
| KNSL.20-6s | AAACATGAGATGGTCCTTTCTA | 346 | se- |
| | | | quencing |

Polymorphic regions (such as SNPs, and the like) were discovered by comparing the sequenced samples to the reference KNSL1 sequence (SEQ ID NO:347) and identifying nucleotides that varied from the reference nucleotide sequence at specific positions. SEQ ID NO:347 represents the reverse complement of a 63,824 nucleotide portion of NCBI Accession# NT_{—}008769.1 starting at nucleotide 1,669,312 and ending at nucleotide 1,733,136. Table 6 shows the polymorphic regions that were identified, including previously identified polymorphic regions identified (set forth as a yes in the public data column), and the type of nucleotide polymorphic change detected relative to the KNSL1 reference sequence set forth as SEQ ID NO:347.

TABLE 6
(KNSL1 Polymorphic Regions)

| | POSITION IN | POSITION IN | | NUCLEOTIDE |
|---|---|---|---|---|
| POLYMORPHISM | SEQ | SEQ ID | PUBLIC | CHANGE |
| NAME | ID NO: 347 | NO: 484 | DATABASE | DETECTED |
| US-565 | 300 | 138886 | NO | Insertion of -CA- |
| | | | | or of a 4-, 8-, |
| | | | | 10- or 14-bp -CA- |
| | | | | repeat or |
| | | | | Deletion of -CA- |
| | | | | between 13884 |
| | | | | and 13887 |
| 1i.D + 69 | 1152 | 139739 | NO | G → T |
| 2i.A-16 | 14235 | 152822 | NO | Insertion of T |
| 4i.D + 236 | 15104 | 153691 | NO | A → G |
| 7i.D + 55 | 20815 | 159403 | NO | T → C |
| 10i.A-212 | 36738 | 174814 | NO | CA → AC |
| 13i.D + 70 | 41015 | 178981 | NO | 5nt Insertion |
| | | | | between |
| | | | | positions 41014 |
| | | | | and 41015 |
| 14i.D + 78 | 42125 | 180091 | NO | T → G |
| 18e.CDS + 2605 | 56706 | 194487 | NO | C → T |
| 18i.D + 16 | 56887 | 194668 | NO | A → G |
| 19i.D + 57 | 58524 | 196261 | NO | C → T |

Upstream Sequence

Several polymorphisms were discovered upstream of the transcription start site of the KNSL1 gene. The polymorphism designated US-7082 occurs 7082 nucleotides upstream of the transcription start site (see nucleotide 132,370 of SEQ ID NO:484). The polymorphic region designated US-6097 corresponds to the presence or absence of an insertion of an additional 6, 7 or 8 poly-T nucleotide between nucleotides 133354-133355 of SEQ ID NO:484, which corresponds to the insertion at a position 6097 nucleotides 5′ to the transcription start site. This particular poly-T insertion follows the poly-T nucleotide sequence corresponding to nucleotides 133341-133354 of SEQ ID NO:484. The polymorphic region designated US-565 corresponds to an insertion of an additional dinucleotide -CA- between any of the -CA- dinucleotides at nucleotides 270-299 of SEQ ID NO:347, such as between nucleotides 299 and 300 of SEQ ID NO:347. In one embodiment, the -CA- nucleotide insertion occurs at a position 565 nucleotides 5′ to the transcription start site. Thus, the allele corresponding to SEQ ID NO:347 has a 15 dinucleotide (-CA-) repeat corresponding to nucleotides 270 to 299, while the US-565 allele identified herein has a 16 dinucleotide (-CA-) by the addition of a dinucleotide -CA- between any one of the 15 dinucleotide repeats, such as nucleotides 299 and 300 of SEQ ID NO:347. These polymorphisms are located in putative promoter or enhancer regions of the KNSL1 gene and may affect the expression of the KNSL1 gene.

Intervening Sequences

Several polymorphic regions corresponding to SNPs, single-nucleotide insertions, dinucleotide inversions, multi-nucleotide insertions, were identified herein in introns when compared to SEQ ID NO:347. For example, polymorphic region 1i.D+69 (at position 1152 of SEQ ID NO:347) corresponds to a SNP located in the intron between exon 1 and exon 2, 69 nucleotides 3′ of the splice donor site. The polymorphic region labeled 1i.A-4641 corresponds to a 4-nucleotide insertion occurring between exons 1 and 2, 4641 nucleotides 5′ of the splice acceptor site of exon 2, where the oligonucleotide AGTT is inserted at a position corresponding to nucleotides 147842-147845 of SEQ ID NO:484. Polymorphic region 2iA.-16 corresponds to an insertion of a T nucleotide after any one of the poly-T nucleotides at positions 14222 through 14234 of SEQ ID NO:347. Nucleotide 14234 is 16 nucleotides 5′ of the splice acceptor site of exon 3. Polymorphic region corresponding to the SNP labeled 4i.D+236 (nucleotide 15104 of SEQ ID NO:347) is located between exons 4 and 5, 236 nucleotides 3′ of the splice donor site. Polymorphic region corresponding to the SNP labeled 7i.D+55 (nucleotide 20815 of SEQ ID NO:347) is located between exons 7 and 8, 55 nucleotides 3′ of the splice donor site. The polymorphic region labeled 10i.A-212 corresponds to a dinucleotide inversion occurring between exons 10 and 11, where the dinucleotide CA- at positions 36738-36739 of SEQ ID NO:347 is replaced with the inverted -AC- dinucleotide at the same nucleotide positions. The polymorphic region labeled 13i.D+70 corresponds to a 5-nucleotide insertion occurring between exons 13 and 14, 70 nucleotides 3′ of the splice donor site, where the oligonucleotide -AATTT- is inserted between nucleotides 41014 and 41015 of SEQ ID NO:347, which also corresponds to between nucleotides 17890-178981 of SEQ ID NO:484. Polymorphic region corresponding to the SNP labeled 14i.D+78 (nucleotide 42125 of SEQ ID N0:347) is located between exons 14 and 15, 78 nucleotides 3′ of the splice donor site. Polymorphic region corresponding to the SNP labeled 18i.D+16 (nucleotide 56887 of SEQ ID NO:347) is located between exons 18 and 19, 16 nucleotides 3′ of the splice donor site. Polymorphic region corresponding to the SNP labeled 19i.D+57 (nucleotide 58524 of SEQ ID NO:347) is located between exons 18 and 19, 16 nucleotides 3′ of the splice donor site. These polymorphisms are contemplated herein as potentially affecting the splicing of the KNSL1 RNA.

Coding Sequence

Polymorphic region corresponding to the SNP labeled 18e.CDS+2605 (nucleotide 56706 of SEQ ID NO:347) is located in exon 19 and results in an amino acid change from R to C at position amino acid 869 of KNSL1 (see SEQ ID NOs:471-476 provided herein). GenBank Accession numbers XM_{—}005889, XM_{—}051151 and XM_{—}051152 denote well-known sequences for a KNSL1 cDNA.

Table 6-B shows additional KNSL1 polymorphic regions that were identified, including previously identified polymorphic regions (set forth as a yes in the public database column), and the type of nucleotide polymorphic change detected relative to the KNSL reference genomic sequence that corresponds to approximately nucleotides 130,000 to the end of SEQ ID NO:484.

TABLE 6-B

| | | | NUCLEOTIDE |
|---|---|---|---|
| POLYMORPHISM | POSITION IN SEQ ID | PUBLIC | CHANGES |
| NAME | NO: 484 | DATABASE? | DETECTED |
| KNSL1_us-8576 | 130876 | NO | C→T |
| KNSL1_us-8074 | 131378 | NO | A→G |
| KNSL1_us-7836 | 131616 | NO | A→G |
| KNSL1_us-7832 | 131620 | NO | A→G |
| KNSL1_us-7764 | 131688 | NO | T→G |
| KNSL1_us-7454 | 131998-132003 | NO | Deletion of -CTTTTC- |
| KNSL1_us-7448 | 132004 | NO | 29 bp poly-T |
| | | | Repea→9, 16, 21 or |
| | | | 26 bp poly-T Repeat |
| KNSL1_us-7082 | 132370 | YES | G→A |
| KNSL1_us-6755 | 132697 | NO | A→G |
| KNSL1_us-6484 | 132968 | NO | C→T |
| KNSL1_us-6097 | 133355 | NO | 6, 7 or 8 base pair |
| | | | poly-T Repeat |
| | | | Insertion between |
| | | | positions 133354 |
| | | | and 133355 |
| KNSL1_us-5646 | 133806 | NO | T→G |
| KNSL1_us-5422 | 134030 | NO | G→A |
| KNSL1_us-5161 | 134291 | NO | G→A |
| KNSL1_us-4791 | 134661 | NO | G→A |
| KNSL1_us-2365 | 137087 | NO | A→G |
| KNSL1_us-2310 | 137142 | NO | G→A |
| KNSL1_us-1056 | 138396 | NO | C→T |
| KNSL1_1i.D + 995 | 140665 | NO | T→G |
| KNSL1_1i.D + 1066 | 140736 | NO | A→G |
| KNSL1_1i.D + 1503 | 141173 | NO | A→G |
| KNSL1_1i.D + 2386 | 142056 | NO | T→C |
| KNSL1_1i.D + 3107 | 142777 | NO | -AG- Insertion |
| KNSL1_1i.D + 3355 | 143025 | NO | T→G |
| KNSL1_1i.D + 4059 | 143729 | NO | C→A |
| KNSL1_1i.D + 4814 | 144484 | NO | A→T |
| KNSL1_1i.A-6302 | 146181 | NO | T→A |
| KNSL1_1i.A-5432 | 147051 | NO | G→A |
| KNSL1_1i.A-5160 | 147322 | NO | C→T |
| KNSL1_1i.A-4775 | 147707 | NO | G→T |
| KNSL1_1i.A-4641 | 147842 | NO | Deletion of -AGTT- |
| KNSL1_1i.A-4403 | 148080 | NO | C→T |
| KNSL1_1i.A-3455 | 149026 | NO | 18 bp -AC- |
| | | | Repeat→17, 19 or |
| | | | 22 bp -AC- Repeat |
| KNSL1_1i.A-3439 | 149044 | NO | 30 bp -GT- |
| | | | Repeat→22, 24, 28, |
| | | | 32 or 36 bp -GT- |
| | | | Repeat |
| KNSL1_1i.A-3094 | 149389 | NO | G→A |
| KNSL1_1i.A-2480 | 150003 | NO | G→A |
| KNSL1_1i.A-2099 | 150384 | NO | G→T |
| KNSL1_1i.A-2029 | 150454 | NO | C→T |
| KNSL1_1i.A-1797 | 150686 | NO | G→T |
| KNSL1_1i.A-1140 | 151343 | NO | C→T |
| KNSL1_1i.A-522 | 151961 | NO | C→T |
| KNSL1_1i.A-364 | 152119 | NO | C→T |
| KNSL1_4i.6 | 153791 | NO | C→G |
| KNSL1_4i.5 | 154328 | NO | A→T |
| KNSL1_4i.4 | 154513 | NO | C→A |
| KNSL1_4i.3 | 154639 | NO | G→A |
| KNSL1_4i.1 | 155049 | NO | T→C |
| KNSL1_4i.2 | 155114 | NO | T→C |
| KNSL1_6i.1 | 158040 | NO | C→A |
| KNSL1_6i.2 | 158895 | NO | G→A |
| KNSL1_17i.1 | 191284 | NO | C→T |
| KNSL1_18i.1 | 192272 | NO | C→T |
| KNSL1_18i.2 | 192698 | NO | A→T |
| KNSL1_18i.3 | 193706 | NO | T→A |

Other known SNPs in the KNSL1 gene contemplated for use in the various diagnostic and screening methods, as well as kits and solid supports, provided herein include the NCBI SNPs set forth in Table C and Table C-2, which are referenced by their respective locations in FIG. 3 and SEQ ID NO:347; and in FIG. 6 and SEQ ID NO:484, respectively.

TABLE C
KNSL1 NCBI Polymorphisms

| | | POSITION IN |
|---|---|---|
| NCBI SNP ID NO. | POLYMORPHISM | SEQ ID NO: 347 |
| rs1573051 | C/T | 35719 |
| rs1972360 | C/T | 45083 |
| rs1547818 | C/G | 45887 |
| rs1044146 | C/T | 62661 |
| rs1044153 | A/C | 63802 |

TABLE C-2
KNSL1 NCBI Polymorphisms

| NCBI SNP | | |
|---|---|---|
| ID NO. | POLYMORPHISM | POSITION IN SEQ ID NO: 484 |
| rs2421941 | G/A | 132370 |
| rs1889894 | A/G | 136968 |
| rs2297743 | G/C | 139284 |
| rs2275220 | A/G | 159167 |
| rs2275219 | T/C | 159403 |
| rs1573051 | C/T | 173795 |
| rs2275217 | C/T | 178748 |
| rs3051599 | ACAG/— | 180149 |
| rs1972360 | C/T | 183049 |
| rs2421946 | A/T | 180153 |
| rs1547818 | C/G | 183853 |
| rs1044146 | C/T | 200398 |
| rs1044153 | A/C | 201539 |

Amplification and Genotyping KNSL1 Haplotype Polymorphism:

As set forth herein, an exemplary haplotype useful in the methods provided herein for determining a predisposition or occurrence of neurodegenerative disease, such as Alzheimer's disease, comprises multiple polymorphic regions of the KNSL1 gene corresponding to nucleotides 132370, 133355, 147842 and 178981 of SEQ ID NO:484. In a particular embodiment, the nucleotide(s) detected in this particular KNSL1 haplotype: at position 132370 of SEQ ID NO:484 is A (also referred to herein as KNSL US-7082); between the nucleotides at positions 133354-133355 of SEQ ID NO:484 is the presence of a 6, 7 or 8 base pair poly-T insertion corresponding to -TTTTTT(T)(T)- that follow the poly-T sequence at nucleotides 133341-133354 of SEQ ID NO:484 (also referred to herein as KNSL US-6097); beginning at position 147842-147845 of SEQ ID NO:484 is the presence of a 4 base pair insertion corresponding to -AGTT- (also referred to herein as KNSL 1i.A-4641), and between the nucleotides at positions 178980-178981 of SEQ ID NO:484 is the presence of a 5 base pair insertion corresponding to -AATTT- (also referred to herein as KNSL 13i.D+70). This -AATTT- insertion immediately follows the -AATTT- sequence corresponding to nucleotides 178976-178980 in SEQ ID NO:484.

The polymorphic regions of the KNSL1 gene corresponding to nucleotides 132370, 133355, 147842 and 178981 of SEQ ID NO:484 can be genotyped using well known methods and the PCR amplification and FP-SBE primers set forth in FIG. 7. For example, all genotypes were generated either using fluorescent polarization detected single base extension (FP-SBE, “Criterion Analyst AD”, Molecular Devices, Inc.), single base extension using capillary electrophoresis (using the “SNuPe” software on a “MegaBACE-1000” genotyping/sequencing system, Amersham-Pharmacia) or by capillary electrophoresis of PCR products using fluorescently labeled primers (using the “Genetic Profiler” software on the “MegaBACE 1000”). Generally, PCR primers were designed to yield products between 200-400 bp in length and added to ˜10 ng of genomic DNA using individually optimized PCR conditions. PCR primers and unincorporated dNTPs were degraded by the direct addition of exonuclease I (0.1-0.15 U/rxn) and shrimp alkaline phosphatase (1 U/rxn). The single base extension step was carried out using Thermosequenase (0.4 U/rxn) and the appropriate mix of R110-ddNTP, TAMRA-ddNTP (3 mM), and all four unlabeled ddNTPs (22 or 25 μM) to the Exol/SAP treated PCR product. To assess genotyping quality, 10% of the samples were randomly duplicated and called twice. Primer sequences, PCR and SBE cycling conditions are set forth in FIG. 7.

TNFRSF6

The nucleotide sequence of the TNFRSF6 gene in the “D10-E16” set was determined. The “D10-E16” set was derived from sixteen genomic DNA samples from four families showing the highest LOD scores for linkage with LOAD at 6 markers (D10S564, D10S583, D10S1710, D10S566, D10S1671 and D10S1741). The set contained individuals affected with AD and individuals who were unaffected.

Genomic sequence was from AL157394 (GenBank Accession number). Exon-intron structure was determined, as previously described, from cDNA sequences from GenBank Accession numbers XM 048187, XM_{—}048189, XM_{—}048190, XM_{—}048193 and XM_{—}048194.

Based on this information, primers were designed to amplify regions of interest from genomic DNA and to sequence these regions on both strands. Amplification and sequencing primers are shown in Table 7.

TABLE 7

Primers for TNFRSF6 Genomic PCR and Sequencing

| | | | SEQ |
|---|---|---|---|
| | | | ID |
| PRIMER NAME | SEQUENCE | PURPOSE | NO: |
| TNFRSF6.pro.p1 | CTT CCT CAT GGC ACT AAC AGT C | PCR | 349 |
| TNFRSF6.pro.p2 | AAG TCA CTC GTA AAC CGC TTC C | PCR | 350 |
| TNFRSF6.pro.s1 | AAC AGA GAC AAG CCT ATC AA | Sequencing | 351 |
| TNFRSF6.pro.s2 | TAT TGC TTT GGA ACG GTA GA | Sequencing | 352 |
| TNFRSF6.pro.s3 | CCA AAC ATA CCT TCT GTA AA | Sequencing | 353 |
| TNFRSF6.pro.s4 | ACA AAT GGG CAT TCC TGT C | Sequencing | 354 |
| TNFRSF6.pro.s5 | GGA CAG CCC AGT CAA ATG | Sequencing | 355 |
| TNFRSF6.pro.s6 | GGG AGG GCT CCA TTG ATT | Sequencing | 356 |
| TNFRSF6.exon01.p1 | GGA GGC CGG CTC TCG AGG TC | PCR | 357 |
| TNFRSF6.exon01.p2 | GGG TCA GCA CTT CGC ATC AA | PCR | 358 |
| TNFRSF6.exon01.s1 | GAC AGG AAT GCC CAT TTG | Sequencing | 359 |
| TNFRSF6.exon01.s2 | CAA GTC CTC CAG CGT TCT | Sequencing | 360 |
| TNFRSF6.exon02.p1 | CCA CAA ACA CAG GGC AGT AAG T | PCR | 361 |
| TNFRSF6.exon02.p2 | TCT CAT TTC AGA GGT GCA TGT C | PCR | 362 |
| TNFRSF6.exon02.s1 | AGT GGA GCC CTC ACA TTG TC | Sequencing | 363 |
| TNFRSF6.exon02.s2 | ATA AAT AAA ACT CAT CTT TGG A | Sequencing | 364 |
| TNFRSF6.exon03.p1 | TGT ATG CTC CTG TTG CTA ATC A | PCR | 365 |
| TNFRSF6.exon03.p2 | GCA CCA TTA TAT AAG CGT AAG T | PCR | 366 |
| TNFRSF6.exon03.s1 | TAC CTG CCC GTG TCC TGT | Sequencing | 367 |
| TNFRSF6.exon03.s2 | TGG AAG AAT TGC CTA GAC T | Sequencing | 368 |
| TNFRSF6.exon04.p1 | GTA TTG GCC TGA TGG AGT AAG T | PCR | 369 |
| TNFRSF6.exon04.p2 | TAA CAC CTG GAA CAT AAG TCT C | PCR | 370 |
| TNFRSF6.exon04.s1 | TAG AGG AAC AGG GGA GAC G | Sequencing | 371 |
| TNFRSF6.exon04.s2 | TGC TTC TGG ACT GAT ACC TT | Sequencing | 372 |
| TNFRSF6.exon05.p1 | CAG AGT CAT TCC TTA TGA TAT T | PCR | 373 |
| TNFRSF6.exon05.p2 | GGC AAA TCT TTG TGA ACT ACT T | PCR | 374 |
| TNFRSF6.exon05.s1 | TTG TGC CAG CTT TAG ATA C | Sequencing | 375 |
| TNFRSF6.exon05.s2 | AAA GCC ACC CCA AGT TAG AT | Sequencing | 376 |
| TNFRSF6.exon06.p1 | CTA ATT TAC AAA GTG CCA TTG A | PCR | 377 |
| TNFRSF6.exon06.p2 | CAT AGG AAG GAT GAG AAT ACC A | PCR | 378 |
| TNFRSF6.exon06.s1 | GTA ATG GGC AGA GGC TGT G | Sequencing | 379 |
| TNFRSF6.exon06.s2 | CAG GGG CAA GAA ATT AAC A | Sequencing | 380 |
| TNFRSF6.exon07.p1 | ATG TCC TTT CAC TAG AAC ACT T | PCR | 381 |
| TNFRSF6.exon07.p2 | TAG TCC CAG GGT CAC AGT TGA G | PCR | 382 |
| TNFRSF6.exon07.s1 | AGC GGT CTC CTG CGA TGT | Sequencing | 383 |
| TNFRSF6.exon07.s2 | CTT GAG CCC AGG ACT TTG | Sequencing | 384 |
| TNFRSF6.exon08.p1 | CTT GCC TTT AAA AAC TAA GAC A | PCR | 385 |
| TNFRSF6.exon08.p2 | GCA GGA GTT CTG TAA TAC ATC T | PCR | 386 |
| TNFRSF6.exon08.s1 | CCT AAT TTT ATA CAT CAA GCA | Sequencing | 387 |
| TNFRSF6.exon08.s2 | TAA ACA AAC TGC CTG ATA AA | Sequencing | 388 |
| TNFRSF6.exon09.p1 | ACA GAA ATG TTA TGT ATT GTG G | PCR | 389 |
| TNFRSF6.exon09.p2 | ACA CTC CTA TTT GGG TAC TTA G | PCR | 390 |
| TNFRSF6.exon09.s1 | ATG GTT TTC ACT AAT GGG A | Sequencing | 391 |
| TNFRSF6.exon09.s2 | CTA CAA ATA TGT TGG CTC TTC | Sequencing | 392 |
| TNFRSF6.3′UTR.p1 | GAA AGA AAG AAG CGT ATG ACA C | PCR | 393 |
| TNFRSF6.3′UTR.p2 | CCA AAA AGC AAG CCC CTA CCA A | PCR | 394 |
| TNFRSF6.3′UTR.s1 | GAA GCG TAT GAC ACA TTG AT | Sequencing | 395 |
| TNFRSF6.3′UTR.s2 | TCT ACA AAT ATG TTG GCT CT | Sequencing | 396 |
| TNFRSF6.3′UTR.s3 | ACC CCA CTC TAT GAA TCA A | Sequencing | 397 |
| TNFRSF6.3′UTR.s4 | CAA GGC AAA AAT GGA GAG | Sequencing | 398 |
| TNFRSF6.3′UTR.s5 | AAA TAA GGC TCT ACC TCA AA | Sequencing | 399 |
| TNFRSF6.3′UTR.s6 | AAT GGA AGT TCT TTA GGT GG | Sequencing | 400 |
| TNFRSF6.3′UTR.s7 | CCC CGA AAA TGT TCA ATA | Sequencing | 401 |

Several polymorphic regions were identified in the human TNFRSF6 gene. These are listed in Table 8. These were discovered by comparing the sequenced samples to the reference TNFRSF6 sequence (SEQ ID NO:402). SEQ. ID NO:402 represents the reverse complement of a 2818 nucleotide portion of AL157394.11 starting at nucleotide 17,215 and ending at nucleotide 45,332. Table 8 shows the polymorphic regions that were identified, including those previously identified (set forth as yes in public data column), the position in SEQ ID NO:402 and the nucleotide change detected relative to the reference sequence.

TABLE 8
TNFRSF6 Polymorphic Regions

| | NUCLEOTIDE | POSITION | |
|---|---|---|---|
| POLYMORPHISM | CHANGE | IN SEQ ID | PUBLIC |
| NAME | DETECTED | NO: 402 | DATABASE? |
| US-470 | T → C | 1530 | Yes |
| US-450 | A → G | 1550 | Yes |
| us.3 | G → A | 1926 | No |
| 1i.1 | G → A | 2269 | No |
| 2e.CDS + 183 | G → A | 14525 | No |
| 2i.D + 176 | C → T | 14714 | No |
| 2i.2 | C → T | 18934 | Yes |
| 2i.A-62 | G → C | 18982 | Yes |
| 3e.CDS + 222 | A → G | 19069 | Yes |
| 3i.1 | C → T | 19227 | Yes |
| 4i.D + 71 | A → G | 20412 | No |
| 4i.D + 211 | A → G | 20552 | No |
| 5i.1 | C → G | 22026 | Yes |
| 6i.A-144 | G → A | 23199 | No |
| 7e.CDS + 642 | T → C | 23416 | Yes |
| 8i.D + 179 | A → G | 24890 | Yes |
| 9e.3′UTR + 564 | A → T | 26359 | Yes |

Upstream Sequence

Several polymorphisms were discovered upstream of the transcription start site of the TNFRSF6 gene (nucleotide 2001 of SEQ ID NO:402); US-470 (470 nucleotides upstream) and US-450 (450 nucleotides upstream). These polymorphisms are located in putative promoter or enhancer regions of the TNFRSF6 gene and as such a nucleotide change may affect the expression of the TNFRSF6 gene.

Intervening Sequences

2i.D+176 and 2i.A-62 are located in the intron between exon 2 and exon 3, 176 nucleotides 3′ of the splice donor site and 62 nucleotides 5′ of the splice acceptor site, respectively; 4i.D+71 and 4i.D+211 are located in the intron between exon 4 and exon 5, 71 nucleotides and 211 nucleotides 3′ of the splice donor site, respectively; 6i.A-144 is located in the intron between exon 6 and exon 7, 144 nucleotides 5′ of the splice acceptor site; 8iD+179 is located in the intron between exon 8 and exon 9, 179 nucleotides 3′ to the splice donor site. These polymorphisms may affect splicing of the TNFRSF6 RNA.

Coding Sequence

2e.CDS+183 is located in the second exon of the coding region and does not result in an amino acid change; 3e.CDS+222 is positioned in the third exon of the coding region and does not result in an amino acid change; 7e.CDS +642 is located in the seventh exon of the coding region and does not result in an amino acid change. However, these changes may affect splicing, codon usage or mRNA stability. SEQ ID NO:479 denotes sequence for a TNFRSF6 cDNA, based on GenBank Accession number XM_{—}048190. 2e.CDS+183, 3e.CDS+222 and 7e.CDS+642 are located at nucleotide positions 403, 442 and 862 of SEQ ID NO:479, respectively. SEQ ID NOs: 477, 478, 480 and 481 denote also sequence for other TNFRSF6 cDNAs, based on Genbank Accession numbers XM_{—}048187, XM_{—}048189, XM_{—}048193 and XM_{—}048194, respectively. 2e.CDS+183 and 7e.CDS+642 are located at nucleotide positions 208 and 420 of SEQ ID NO:477, respectively. 2e.CDS+183, 3e.CDS+222 and 7e.CDS+642 are located at nucleotide positions 377, 416 and 836 of SEQ ID NO:478, respectively. 2e.CDS+183, 3e.CDS+222 and 7e.CDS+642 are located at nucleotide positions 208, 247 and 604 of SEQ ID NO:480, respectively. 2e.CDS+183 and 3e.CDS+222 are located at nucleotide positions 208 and 247 of SEQ ID NO:481, respectively.

3′ UTR Sequence

9e.3′UTR+564 is located in the 3′ untranslated sequence, 564 nucleotides 3′ of the stop codon and may affect RNA stability or processing, polyadenylation of the TNFRSF6 transcript, etc. 9e.3′UTR+564 is located at nucleotide position 1766 of SEQ ID NO:478 and nucleotide position 1792 of SEQ ID NO:479. Other known SNPs in the TNFRSF6 gene contemplated for use in the various diagnostic and screening methods, as well as kits and solid supports, provided herein include the NCBI SNPs set forth in Table D, which are referenced by their respective locations in FIG. 4 and in SEQ ID NO:402.

TABLE D
TNFRSF6 NCBI Polymorphisms

| | | POSITION IN |
|---|---|---|
| NCBI SNP ID NO. | POLYMORPHISM | SEQ ID NO: 402 |
| rs1926198 | A/G | 199 |
| rs1926197 | A/G | 213 |
| rs2234767 | A/G | 843 |
| rs4064 | G/C | 2967 |
| rs1324551 | C/T | 3103 |
| rs1926196 | C/T | 5335 |
| rs1926195 | A/G | 5345 |
| rs2031610 | C/T | 6074 |
| rs1571011 | A/C | 9374 |
| rs1571012 | C/T | 9907 |
| rs1571013 | A/G | 9936 |
| rs2147421 | C/T | 10937 |
| rs2147420 | A/G | 11200 |
| rs1159120 | C/T | 11279 |
| rs2148287 | C/T | 11359 |
| rs2147419 | G/T | 11503 |
| rs2148286 | A/G | 11511 |
| rs2417418 | C/T | 11587 |
| rs2182408 | C/T | 11694 |
| rs1926194 | A/G | 11905 |
| rs1926193 | C/T | 12193 |
| rs1926192 | A/G | 12208 |
| rs1926191 | A/G | 12238 |
| rs2031613 | C/T | 18511 |
| rs2031612 | C/T | 18567 |
| rs1926190 | A/G | 20640 |
| rs982764 | C/T | 21585 |
| rs1571020 | C/T | 22439 |
| rs1977389 | G/T | 25081 |
| rs1468063 | C/T | 26878 |
| rs1800623 | A/G | 27670 |

LIPA

The nucleotide sequence of the LIPA gene in the “D10-E16” set was determined. The “D10-E16” set was derived from sixteen genomic DNA samples from four families showing the highest combined LOD scores for linkage with LOAD at 6 markers (D10S564, D10S583, D10S1710, D10S566, D10S1671 and D10S1741). The set contained individuals affected with AD and unaffected individuals. Genomic sequence from AL513533.8 and AL353751.11 (GenBank accession numbers) was used to identify exons and design primers. Exon-intron structure was determined, as previously described, from cDNA sequence from NM_{—}000235.

Based on this information, primers were designed to amplify regions of interest from genomic DNA and to sequence those regions on both strands. Amplification and sequencing primers are shown in Table 9.

TABLE 9

Primers for LIPA Genomic PCR and Sequencing

| | | | SEQ |
|---|---|---|---|
| | | | ID |
| NAME | SEQUENCE (5′ → 3′) | PURPOSE | NO: |
| LIPA01C-F1 | CATATCATCCTCCTTATCCCT | PCR | 404 |
| LIPA01C-F2 | TGGCATGATCTCCGCTCACT | Se- | 405 |
| | | quencing | |
| LIPA01C-R1 | TCTATAAATGAAAGTGAATCATGG | PCR | 406 |
| LIPA01C-R2 | GAAAGTGAATCATGGGCTCC | Se- | 407 |
| | | quencing | |
| LIPA01B-F1 | GGTTTCTATACCTCCACTGTCTTAC | PCR | 408 |
| LIPA01B-F2 | TGCCTGGCATTTAGTAGGTG | Se- | 409 |
| | | quencing | |
| LIPA01B-R1 | GACTATTTGTTCAGCCAATCAG | PCR | 410 |
| LIPA01B-R2 | CGGGAAGTGCCTCGGAGACT | Se- | 411 |
| | | quencing | |
| LIPA01-F1 | CGCCTAAAACAGCCTTTGCTAAGA | PCR | 412 |
| LIPA01-F2 | GACGCGCTGGTAGAGCTGT | Se- | 413 |
| | | quencing | |
| LIPA01-R1 | CTGAAGTTGCCCTGGTTGATTG | PCR | 414 |
| LIPA01-R2 | TGAGCCAACGCCCGCAGAAA | Se- | 415 |
| | | quencing | |
| LIPA02-F1 | GCAAGACTCCATCTCAACA | PCR | 416 |
| LIPA02-F2 | GACTCCATCTCAACATAAAT | Se- | 417 |
| | | quencing | |
| LIPA02-F3 | ACTTGCCCTAAATCTGGTTC | Se- | 418 |
| | | quencing | |
| LIPA02-R1 | GGTGAAACCCTGTATCTACTAAAT | PCR | 419 |
| LIPA02-R2 | CGCAATGAGCCGAGATCACA | Se- | 420 |
| | | quencing | |
| LIPA02-R3 | TAACTGGATCGGGGAAATAG | Se- | 421 |
| | | quencing | |
| LIPA03-F1 | GCCCAAGATGTGGTAAAGT | PCR | 422 |
| LIPA03-F2 | ACCGGTAGGTGGAACACAAG | Se- | 423 |
| | | quencing | |
| LIPA03-R1 | TAGCATATCCTCAAATGAATCTG | PCR | 424 |
| LIPA03-R2 | AATGAATCTGGCCTTTGAAC | Se- | 425 |
| | | quencing | |
| LIPA04-F1 | TTTTGCTGCTTACATAAGTGTG | PCR | 426 |
| LIPA04-F2 | TGCTACCTTGCCAGTGCTGT | Se- | 427 |
| | | quencing | |
| LIPA04-R1 | CCAGAAATGCCGAAGTAAC | PCR | 428 |
| LIPA04-R2 | GCCGAAGTAACTTAAGAATT | Se- | 429 |
| | | quencing | |
| LIPA05-F1 | ACTACCCAAATGCATGTCA | PCR | 430 |
| LIPA05-F2 | CTTTTGGGAGACACAACTGG | Se- | 431 |
| | | quencing | |
| LIPA05-R1 | TTCTACTCTCTCACATCCCTATCT | PCR | 432 |
| LIPA05-R2 | CATCCCTATCTTGCTTCATC | Se- | 433 |
| | | quencing | |
| LIPA06-F1 | GAGATTTGGAGCAAGCATTAAC | PCR | 434 |
| LIPA06-F2 | TGTTAGGGCACACGGAAGTT | Se- | 435 |
| | | quencing | |
| LIPA06-R1 | AGGTGGGCAGAGCTGTACT | PCR | 436 |
| LIPA06-R2 | ATGAGTACACGTGGCACCAG | Se- | 437 |
| | | quencing | |
| LIPA06-R3 | CATAGGGCTAGTACAGAAGG | Se- | 438 |
| | | quencing | |
| LIPA07-F1 | TCCCACTGTAGAAGTCCG | PCR | 439 |
| LIPA07-F2 | GTAGAAGTCCGCTGAAAACT | Se- | 440 |
| | | quencing | |
| LIPA07-R1 | GACAGATCTCCTCATTCAATAAT | PCR | 441 |
| LIPA07-R2 | ACCACAGTCAGCCTGAGGAT | Se- | 442 |
| | | quencing | |
| LIPA07-R3 | AATCAAATCTTACTATAAACATGC | Se- | 443 |
| | | quencing | |
| LIPA08-F1 | CCCACACAGTCCCGTGCTTA | PCR | 444 |
| LIPA08-F2 | TCCCGTGCTTACCATGTTGT | Se- | 445 |
| | | quencing | |
| LIPA08-R1 | CCCAGTAGCTGTTTGTGAACCC | PCR | 446 |
| LIPA08-R2 | TAACACAGTGTGCCCCACCA | Se- | 447 |
| | | quencing | |
| LIPA09-F1 | CAGGAGCAACTGTTGATCTAGTAT | PCR | 448 |
| LIPA09-F2 | GCTGCTTTCTTGTGTCAGGT | Se- | 449 |
| | | quencing | |
| LIPA09-R1 | CAGGCTATGTTCGTGATTACTCT | PCR | 450 |
| LIPA09-R2 | CAAAGAGAGGAGGCCCAGTC | Se- | 451 |
| | | quencing | |
| LIPA10A-F1 | GAAGTAGGAGGGCATGACC | PCR | 452 |
| LIPA10A-F2 | CACAGCTAGTGGCGATTAT | Se- | 453 |
| | | quencing | |
| LIPA10A-F3 | AGGACATGCTTGTGCCGACT | Se- | 454 |
| | | quencing | |
| LIPA10A-R1 | ACTGGGCATCTTCAAAGTTATC | PCR | 455 |
| LIPA10A-R2 | AAACAAAAGACCTGGGAAAG | Se- | 456 |
| | | quencing | |
| LIPA10B-F1 | ACCACCAAGTCAATGATTATGTCA | PCR | 457 |
| LIPA10B-F2 | ACTTGTTTTTCTTTCCCAGG | Se- | 458 |
| | | quencing | |
| LIPA10B-R1 | GTATAGTCTCCACAGGGATTTGC | PCR | 459 |
| LIPA10B-R2 | TTTGAAGACGCCGGAAAACT | Se- | 460 |
| | | quencing | |
| LIPA10C-F1 | TGCCAGTAATAAGGATGCTAACAA | PCR | 461 |
| LIPA10C-F2 | TCAAACCTAACTGTGACAGC | Se- | 462 |
| | | quencing | |
| LIPA10C-F3 | TGCCCATGAGAAGTGTCCTT | Se- | 463 |
| | | quencing | |
| LIPA10C-R1 | AAAGGGAAGAACCGGATGA | PCR | 464 |
| LIPA10C-R2 | GAAGAACCGGATGACTGTTA | Se- | 465 |
| | | quencing | |
| LIPA10C-R3 | CCGGACAGTATTGTAAGGAA | Se- | 466 |
| | | quencing | |

Several polymorphic regions have been identified in the human LIPA gene. These are listed in Table 10. These were discovered by comparing the sequenced samples to the reference LIPA sequence corresponding to nucleotides 6,017,146 through 6,057,323 of NT_{—}008679.5 (SEQ ID NO:467). Table 10 shows the polymorphic regioris that were identified, including those previously identified (set forth as yes in public data column), the position in SEQ ID NO:467 and the nucleotide change detected relative to the reference sequence.

TABLE 10
LIPA Polymorphic Regions

| POLYMORPHISM | NUCLEOTIDE | POSITION IN SEQ | PUBLIC |
|---|---|---|---|
| NAME | CHANGE DETECTED | ID NO: 467 | DATABASE? |
| US-703 | C → G | 1197 | No |
| US-593 | ATC → Δ3 | 1307-1309 | Yes |
| US-59 | A → C | 1841 | Yes |
| US-48 | G → A | 1852 | Yes |
| 1i.D + 36 | G → A | 2075 | Yes |
| 1i.A-64 | G → T | 6063 | Yes |
| 2e.CDS + 46 | A → C | 6173 | Yes |
| 2e.CDS + 67 | G → A | 6194 | Yes |
| 2i.A-163 | C → G | 7820 | No |
| 3i.A-95 | G → C | 25283 | Yes |
| 5i.A-95 | TCCGCGAGAGGGC → Δ13 | 28453-28465 | Yes |
| 5i.A-5 | C → T | 28543 | No |
| 6i.D + 62 | A → C | 28746 | No |
| 6i.A-42 | G → A | 29904 | Yes |
| 9i.D + 46 | C → T | 37861 | Yes |
| 10e.3′UTR + 909 | T → A | 39834 | Yes |
| 10e.3′UTR + 1093 | C → T | 40018 | Yes |

Upstream Sequence

Several polymorphisms were discovered upstream of the transcription start site of the LIPA gene (nucleotide 1895 of SEQ ID NO:467); US-703 (703 nucleotides upstream), US-593 (593 nucleotides upstream), US-59 (59 -nucleotides upstream) and US-48 (48 nucleotides upstream). These polymorphisms are located in putative promoter or enhancer regions of the LIPA gene and as such a nucleotide change may affect the expression of the LIPA gene.

Intervening Sequences

1i.D+36 and 1i.A-64 are located in the intron between exon 1 and exon 2, 36 nucleotides 3′ of the splice donor site and 64 nucleotides 5′ of the splice acceptor site, respectively; 2i.A-163 is located in the intron between exon 2 and exon 3, 163 nucleotides 5′ of the splice acceptor site; 3i.A-95 is located in the intron between exon 3 and exon 4, 95 nucleotides 5′ of the splice acceptor site; 5i.A-95 and 5i.A-5 are located in the intron between exon 5 and exon 6, 95 nucleotides 5′ and 5 nucleotides 5′ of the splice acceptor site, respectively; 6i.D+62 and 6i.A-42 are located in the intron between exon 6 and exon 7, 62 nucleotides 3′ of the splice donor site and 42 nucleotides 5′ of the splice acceptor site, respectively; 9i.D+46 is located in the intron between exon 9 and exon 10, 46 nucleotides 3′ of the splice donor site. The polymorphic region labeled 5i.A-95 corresponds to a deletion of nucleotides TCCGCGAGAGGGC at positions 28453-28465 in SEQ ID NO:467. These polymorphisms may affect splicing of the LIPA RNA.

Coding Sequence

2e.CDS+46 and 2e.CDS+67 are located in the second exon of the coding region of the LIPA gene. 2e.CDS+46 results in an amino acid change at residue 16 from T to P. 2e.CDS+67 results in an amino acid change at residue 23 from G to R. These are both missense mutations in a putative 27-residue leader sequence. GenBank Accession number NM_{—}000235 denotes the sequence for a LIPA cDNA (SEQ ID NO:482). 2e.CDS+46 and 2e.CDS+67 are located in SEQ ID NO:482 at nucleotides 86 and 107, respectively.

3′ UTR Sequence

10e.3′UTR+909 and 10e.3′UTR+1093 are located in the 3′ untranslated sequence, 909 and 1093 nucleotides 3′ of the stop codon, respectively and may affect, among other processes, stability, RNA processing, polyadenylation of the LIPA transcript. 10e.3UTR+909 and 10e.3UTR+1093 are located at nucleotides 2149 and 2333, respectively, of SEQ ID NO:482.

Other known SNPs in the LIPA gene contemplated for use in the various diagnostic and screening methods, as well as kits and solid supports, provided herein include the NCBI SNPs set forth in Table E, which are referenced by their respective locations in FIG. 5 and in SEQ ID NO:467.

TABLE E
LIPA NCBI Polymorphisms

| | | POSITION IN |
|---|---|---|
| NCBI SNP ID NO. | POLYMORPHISM | SEQ ID NO: 467 |
| rs928415 | C/T | 7219 |
| rs1930052 | A/G | 8242 |
| rs1332329 | G/T | 10114 |
| rs1412444 | A/G | 10606 |
| rs1320496 | A/G | 10688 |
| rs1412445 | A/G | 10729 |
| rs869820 | C/T | 11559 |
| rs1106211 | A/G | 12031 |
| rs1029074 | C/T | 14497 |
| rs1029073 | A/G | 14729 |
| rs1930053 | C/T | 21145 |
| rs951647 | C/G | 21329 |
| rs951648 | A/G | 21404 |
| rs885561 | A/G | 21429 |
| rs1041388 | C/T | 22246 |
| rs1041389 | A/G | 22354 |
| rs1041390 | C/T | 22621 |
| rs914606 | C/T | 23802 |
| rs2071510 | A/C | 25969 |

Based on the methods disclosed herein, along with other used in the art, one of ordinary skill would be able to determine polymorphisms that are useful for diagnostic and/or therapeutic discovery purposes for neurodegenerative diseases and to determine the affect of the polymorphisms on the uPA, SNCG, IDE, KNSL1, TNFRSF6 or LIPA gene and/or protein using the methods disclosed herein. Any identified polymorphisms, including SNPS, that are not herein denoted, are considered potentially useful in the described methods and kits. Also, one of ordinary skill in the art would be able to identify additional polymorphic regions of the uPA, SNCG, IDE, KNSL1, TNFRSF6 or LIPA gene by sequencing-additional samples and comparing the sequences.

### EXAMPLE 4

Sequencing of the uPA Gene

Genomic sequences were downloaded from the Human Genome Project public database. The exon-intron structure of each candidate gene was determined by querying the NCBI BLASTN search and alignment program with one or more cDNA sequences encoding the gene (Altschul et al. (1997) Nucleic Acids Res. 25:3389-3402). Based on this information, primers were designed to amplify regions of interest from genomic DNA and sequence them on both strands. These regions consisted of: (1) approximately 1 kb of 5′ flanking sequence 5′ to the beginning of exon 1, containing the putative promoter; (2) all exons plus 50-200 bp 5′ and 3′ flanking sequence for each one; and (3) -700 bp 3′ to the translation stop codon. When the final exon contained a 3′UTR >700 nt long, the region >700 nt 3′ to the stop codon was not amplified.

The genomic DNA samples were obtained from NIMH as described in Example 1. The desired regions were amplified by PCR using 30 ng each genomic DNA with the HotStarTaq Master Mix kit (QIAGEN, Inc., Valencia, Calif.) and a final concentration of 1 μm each of specific PCR primers (see Tables below) according to the manufacturer's protocol. The annealing temperature for different primers was varied as required. The reactions were purified using the QIAquick 96 PCR Purification Kit (QIAGEN, Inc., Valencia, Calif.) according to the manufacturer's protocol. PCR product yields were quantitated using the PicoGreen dsDNA Quantitation Kit (Molecular Probes, Inc., Eugene, Oreg.) according to the manufacturer's protocol. Sequencing reactions were performed with ABI PRISM BigDye” Terminators v3.0 Cycle Sequencing Kit (Applied Biosystems, Foster City, Calif.), using a modification of the manufacturer's protocol as follows. For each template and primer combination (see Tables, below), a mixture of 2 μl BigDye™ Mix, 4 μl 5× Sequencing Buffer, 8 μl H₂O, 2 μl primer (1.6 pmol/μl), and 4μl PCR product (3 ng/μl) was subjected to 30 cycles of 10 s at 96° C., 5 s at 50° C. and 4 min at 60° C. The reactions were purified on a Centri-Sep 96 plate (Princeton Separations, Adelphia, N.J.) according to the manufacturer's protocol and analyzed in an ABI 3700 Automated DNA Sequencer (Applied Biosystems, Foster City, Calif.). Sequence data for each region in all samples were aligned using Sequencher software (GeneCodes Corp., Ann Arbor) and manually evaluated for the presence of polymorphisms.

The nucleotide sequence of the uPA gene in the Top10 set+E16 was determined. The Top10 set+E16 was derived from 26 genomic DNA samples from families showing the highest combined LOD scores for linkage with LOAD at 6 markers (D10S564, D10S583, D10S1710, D10S566, D10S1671 and D10S1741). A second set of 24 genomic DNA samples was also sequenced. This set contains 10 families with the best combined LOD scores to marker D10S1432 (92 cM), D10S1218 (5.2 cM), D10S1225 (80.8 cM), D10S1221 (75.6 cM), and D10S1208 (63.0 cM). Both sets contained individuals affected with AD and unaffected individuals.

Primers were designed to amplify regions of interest from genomic DNA and to sequence those regions on both strands. Amplification and sequencing primers are shown in Table 11.

TABLE 11
Primers for uPA gene Genomic PCR and Sequencing

| | | | | SEQ ID |
|---|---|---|---|---|
| REGION | PRIMER NAME | SEQUENCE (5′ → 3′) | PURPOSE | NO: |
| Promoter | B-13 Pma p01 | CTCCAGAACCTCCACAGTTAGAAC | PCR, sequencing | 509 |
| Promoter | B-13 Pma p02 | CGAGAGGCTTGTAAATTCTCCGTG | PCR, sequencing | 510 |
| Promoter | B-13 Pma 5s | CGGCTTTGTTCCATCCACTG | sequencing | 511 |
| Promoter | B-13 Pma 6s | AGAAAAACCCCTGCCTGTAG | sequencing | 512 |
| Promoter | B-13 Pmb p01 | CAAGGGGGTCTGAGGCAGTCTTAG | PCR, sequencing | 513 |
| Promoter | B-13 Pmb p02 | GGAGCTGGGGCCGCCGCGCATCCG | PCR, sequencing | 514 |
| Promoter | B-13 Pmb 5s | TCCTCAGTCCAGACGCTGTT | sequencing | 515 |
| Promoter | B-13 Pmb 6s | CAGCGGAGGGGACCCAACAG | sequencing | 516 |
| Exon 01 | B-13 f01 p01 | GATTCCTCAGTCCAGACGCTGTTG | PCR, sequencing | 517 |
| Exon 01 | B-13 f01 p02 | GGGCTCTCATGGTGGCGAGGTCGG | PCR, sequencing | 518 |
| Exon 02 | B-13 f02 p01 | GTGAGTGCCGCGGTCCTGAGATCC | PCR, sequencing | 519 |
| Exon 02 | B-13 f02 p02 | CCCAAGCAAGCTTCATCTACCAGA | PCR, sequencing | 520 |
| Exon 02 | B-13 f02 5s | TTGCAGAGCCGCCGTCTAGC | sequencing | 521 |
| Exon 03 | B-13 f03 p01 | GGAGGAAGAGGCCGCCGGGACT | PCR, sequencing | 522 |
| Exon 03 | B-13 f03 p02 | TTTGTGTGGCCACTGTTTCGTG | PCR, sequencing | 523 |
| Exon 03 | B-13 f03 5s | CATCCCCTCCCTGCCCTCTG | sequencing | 524 |
| Exon 03 | B-13 f03 6s | GAGGGCAGGGAGGGGATGTC | sequencing | 525 |
| Exon 04 | B-13 f04 p01 | CCAGCCTGGACAACATGGTGTA | PCR, sequencing | 526 |
| Exon 04 | B-13 f04 p02 | ACTCTTGGACAAGCGGCTTTAG | PCR, sequencing | 527 |
| Exon 04 | B-13 f04 5s | GCCCTGCCTGCCCTGGAACT | sequencing | 528 |
| Exon 04 | B-13 f04 6s | GGTGTCAGTGCTGGCCTTTC | sequencing | 529 |
| Exon 05 | B-13 f05 p01 | TCTGCCACTGTCCTTCAGCAAACG | PCR, sequencing | 530 |
| Exon 05 | B-13 f05 p02 | AATTCTCCCCCAATAATCTTAAAG | PCR, sequencing | 531 |
| Exon 05 | B-13 f05 5s | CTAAAGCCGCTTGTCCAAGA | sequencing | 532 |
| Exon 05 | B-13 f05 6s | CTCTTGGACAAGCGGCTTTA | sequencing | 533 |
| Exon 06 | B-13 f06 p01 | GAGAAGTGCGGCCTCTGGTTGAGT | PCR, sequencing | 534 |
| Exon 06 | B-13 f06 p02 | TAGTCCTCCTTCTTTGGGTAATCA | PCR, sequencing | 535 |
| Exon 06 | B-13 f06 5s | CTCCTACCTGCCTCCCTAAG | sequencing | 536 |
| Exon 06 | B-13 f06 6s | AGGGCTGGTTCTCGATGGTG | sequencing | 537 |
| Exon 07 | B-13 f07 p01 | GGCCCTGGGTTTCTCCTCTTCGAC | PCR, sequencing | 538 |
| Exon 07 | B-13 f07 p02 | AGGCTCCATTCTCCAAGTTCTATA | PCR, sequencing | 539 |
| Exon 07 | B-13 f07 5s | CTGACACGCTTGCTCACCAC | sequencing | 540 |
| Exon 07 | B-13 f07 6s | CACTCTCCCCAAGCCATTAT | sequencing | 541 |
| Exon 08 | B-13 f08 p01 | ATGGATTCCAGCCTAACTACCTCA | PCR, sequencing | 542 |
| Exon 08 | B-13 f08 p02 | GTCTGCCAGAGAGGGGAACCATAA | PCR, sequencing | 543 |
| Exon 08 | B-13 f08 5s | CCTGCCCTCGATGTATAACG | sequencing | 544 |
| Exon 08 | B-13 f08 6s | AAACTGGGGATCGTTATACA | sequencing | 545 |
| Exon 09 | B-13 f09 p01 | GAAGAGTGTTGTTTAGGGAGCGAT | PCR, sequencing | 546 |
| Exon 09 | B-13 f09 p02 | ACACCCTGGGCACTGAGTTACATT | PCR, sequencing | 547 |
| Exon 09 | B-13 f09 5s | TTCCTGCCAGGTGAGTGTTC | sequencing | 548 |
| Exon 09 | B-13 f09 6s | TCTGGGGAGATATGGAAGAG | sequencing | 549 |
| Exon 10 | B-13 f10 p01 | CTAATCAGGAGGCTGAGACATGGG | PCR, sequencing | 550 |
| Exon 10 | B-13 f10 p02 | CTTCAGTCCAGAAAAAGACAAGTT | PCR, sequencing | 551 |
| Exon 10 | B-13 f10 5s | GCCGTGGATGTGCCCTGAAG | sequencing | 552 |
| Exon 10 | B-13 f10 6s | TCGTGTAGACGCCTGGCTTG | sequencing | 553 |
| Exon 11 | B-13 f11 p01 | CCACCGGCTTTCTTGCTGGTTGTC | PCR, sequencing | 554 |
| Exon 11 | B-13 f11 p02 | TACCTCCCAAAGCTCCATTAAGTC | PCR, sequencing | 555 |
| Exon 11 | B-13 f11 5s | CGACGGTGGGCATTTGTGAG | sequencing | 556 |
| Exon 11 | B-13 f11 6s | GACCCAAGAGGCCCCAGGAG | sequencing | 557 |
| Exon 11 | B-13 f11 8s | CCTCACAAATGCCCACCGTC | sequencing | 558 |

Several polymorphic regions have been identified in the human uPA gene. Table 12 shows the polymorphic regions that were identified, including those previously identified (set forth as yes in public data column), the position in SEQ ID NO:559 and the nucleotide change detected relative to the reference sequence.

TABLE 12
Urokinase Plasminogen Activator Gene Polymorphic Regions

| | NUCLEOTIDE | | |
|---|---|---|---|
| POLYMORPHISM | CHANGE | POSITION IN | PUBLIC |
| NAME | DETECTED | SEQ ID NO: 559 | DATABASE |
| us-992 | A → C | 9 | Yes |
| | | | rs2227553 |
| US-600 | G → A | 401 | No |
| US-538 | Deletion | 464 | Yes |
| | of G | | |
| US-487 | C → T | 515 | No |
| US-254 | G → T | 748 | No |
| 1i.D + 186 | T → G | 1229 | Yes |
| (US-152) | | | rs1916341 |
| 2e.5′utr-25 | C → T | 1356 | Yes |
| | | | rs2227579 |
| 2i.A-114 | T → C | 1752 | No |
| 3i.D + 49 | G → A | 1942 | Yes |
| | | | rs222758 |
| 4e.CDS + 173 | G → A | 2127 | Yes |
| 4i.D + 396 | G → A | 2543 | Yes |
| | | | rs2227560 |
| 5i.D + 105 | G → A | 3029 | Yes |
| | | | rs2227562 |
| 6e.CDS + 422 | C → T | 3169 | Yes |
| | | | rs2228228, |
| | | | rs2227564 |
| 7i.A-7 | T → C | 3799 | Yes |
| | | | rs2227566 |
| 8e.CDS + 822 | C → T | 3947 | Yes |
| | | | rs2228227, |
| | | | rs2227568 |
| 9i.D + 66 | C → T | 4808 | Yes |
| | | | rs2227571 |
| 10i.D + 62 | T → C | 5287 | Yes |
| | | | rs2227583 |
| 11e.3utr + 141 | T → C | 6532 | Yes |
| | | | rs4065 |

TABLE 12-B
Urokinase Plasminogen Activator Gene Polymorphic Regions

| | NUCLEOTIDE | | |
|---|---|---|---|
| POLYMORPHISM | CHANGE | POSITION IN | PUBLIC |
| NAME | DETECTED | SEQ ID NO: 563 | DATABASE |
| us-1920 | T → C | 79 | Yes |
| us-1907 | C deletion | 93 | No |
| us-1745 | G → T | 256 | Yes |
| | | | rs2227552 |
| us-1616 | C → T | 385 | Yes |
| | | | rs2227552 |
| us-1287 | GT deletion | 714-715 | No |

The polymorphism at position 256 in SEQ ID NO:563 corresponds to the polymorphism at position 82 of SEQ ID NO:564.

Upstream Sequence

Several polymorphisms were discovered upstream of the transcription start site of the uPA gene; US-600 (600 nucleotides upstream), US-538 (538 nucleotides upstream), US-487 (487 nucleotides upstream) and US-254 (254 nucleotides upstream). These polymorphisms are located in putative promoter or enhancer regions of the uPA gene and as such a nucleotide change may affect the expression of the uPA gene.

Intervening Sequences

1i.D+186 is located in the intron between exon 1 and exon 2, 186 nucleotides 3′ of the splice donor site; 2i.A-114 is located in the intron between exon 2 and exon 3, 114 nucleotides 5′ of the splice acceptor site; 3i.D+49 is located in the intron between exon 3 and exon 4, 49 nucleotides 3′ of the splice donor site; 4i.D+396 is located in the intron between exon 4 and exon 5, 396 nucleotides 3′ of the splice donor site; 5i.D+105 is located in the intron between exon 5 and exon 6, 105 nucleotides 3′ of the splice donor site; 7i.A-7 is located in the intron between exon 7 and exon 8, 7 nucleotides 5′ of the splice acceptor site; 9i.D+66 is located in the intron between exon 9 and exon 10, 66 nucleotides 3′ of the splice donor site and 10i.D+62 is located in the intron between exon 10 and exon 11, 62 nucleotides 3′ of the splice donor site. These polymorphisms may affect splicing of the uPA RNA.

Coding Sequence

4e.cds+173 is located in the fourth exon of the uPA gene in the coding region and results in an amino acid change at residue 58 from glycine to glutamine. 6e.cds+422 is located in the sixth exon of the uPA gene in the coding region and results in an amino acid change at residue 140 from proline to leucine. 8e.cds+822 is located in the eighth exon of the uPA gene in the coding region and results in no change of the amino acid residue at position 274 of the protein. Genbank Accession number NM_{—}002658.1 denotes the sequence for a uPA gene cDNA (SEQ ID NO:561). 4e.cds+173, 6e.cds+422 and 8e.cds+822 are represented in SEQ ID NO:561 at nucleotide positions corresponding to positions 249, 498 and 898, respectively.

5′ UTR Sequence

2e.5′utr-25 is located in the 5′ untranslated sequence of the uPA gene, 25 nucleotides 5′ of the start codon and may affect, among other processes translation initiation and RNA stability. 2e.5′utr-25 is represented in SEQ ID NO: 561 at a nucleotide position corresponding to position 49.

3′ UTR Sequence

11e.3utr+141 is located in the 3′ untranslated sequence of the uPA gene, 141 nucleotides 3′ of the stop codon, and may affect, among other processes, stability, RNA processing and/or polyadenylation of the uPA gene transcript. 11e.3utr+141 is represented in SEQ ID NO:561 at a nucleotide position corresponding to position 1512.

Other known polymorphisms in the uPA gene contemplated for use in the various diagnostic and screening methods, as well as kits and solid supports, provided herein include the NCBI polymorphisms set forth in Table F, which are referenced by their respective locations in FIG. 8 and in SEQ ID NO:559.

TABLE F
uPA gene NCBI Polymorphisms

| | | Position |
|---|---|---|
| NCBI SNP ID No. | Polymorphism | in SEQ ID NO: 559 |
| rs2227554 | A/G | 178 |
| rs2227555 | C/A | 1363 |
| rs2227580 | G/T | 1423 |
| rs1916340 | C/A | 1465 |
| rs2227556 | C/T | 1540 |
| rs2227557 | C/T | 2297 |
| rs2227558 | T/G | 2445 |
| rs2227561 | G/A | 2653 |
| rs2227563 | G/A | 3080 |
| rs1050120 | C/G | 3546 |
| rs2227565 | C/T | 3664 |
| rs2229301, rs2227567 | A/C | 3816 |
| rs2227582 | T/C | 4320 |
| rs2227569 | G/A | 4369 |
| rs2227570 | C/A | 4399 |
| rs2227572 | G/A | 4851 |
| rs1050122 | G/A | 5186 |
| rs1130957 | G/A | 5204 |
| rs2227573 | C/G | 5787 |
| rs1050124 | C/G | 6519 |
| rs1804874 | G/T | 6909 |
| rs2227574 | Deletion of G | 7235 |
| rs2227584 | C/T | 7848 |
| rs2227575 | A/C | 7908 |

Based on the methods disclosed herein, along with other used in the art, one of ordinary skill would be able to determine polymorphisms that are useful for diagnostic and/or therapeutic discovery purposes for neurodegenerative diseases and to determine the affect of the polymorphisms on the uPA gene and/or protein using the methods disclosed herein. Any identified polymorphisms, including SNPS, that are not herein denoted, are considered potentially useful in the described methods and kits. Also, one of ordinary skill in the art would be able to identify additional polymorphic regions of the uPA gene by sequencing additional samples and comparing the sequences.

### EXAMPLE 5

Association Analyses

Polymorphisms of human chromosome 10q genes and surrounding regions were analyzed individually and in combinations (haplotypes) for genetic association with Alzheimer's disease (AD). The studies were conducted by genotyping genomic DNA samples from members of AD families that are part of the National Institute of Mental Health (NIMH) Genetics Initiative sample set and conducting family-based association statistical analysis of the genotyping data.

Genotyping was performed using methods described herein, including fluorescence polarization single-base extension detection, single base extension using capillary electrophoresis and capillary electrophoresis of PCR products using fluorescently labeled primers. Genotype data were tested for association with AD in the sample as a whole and in two strata based on onset age using two different statistical programs for family-based tests of association: FBAT (Rabinowitz and Laird (2000) Hum. Hered. 50:211-223) for single-locus analyses and TRANSMIT (Clayton (1999) Am. J. Hum. Genet. 65:1170-1177) for multilocus haplotype analyses. These tests are similar to the transmission/disequilibrium test (TDT) and thus essentially are not susceptible to errors due to population admixture. In addition, these tests allow for missing parental genotypes and are valid in the presence of linkage in families of arbitrary size.

In some studies, association analyses were first conducted on a “screening sample” of discordant sibships (e.g., 189 sibships and 789 individuals) before analysis of the full sample of NIMH families (1439 individuals from 437 pedigrees). The results of the initial analysis of the screening sample were used in determining which polymorphisms would be genotyped in the full sample based on evidence of association.

The results of these analyses revealed genetic association between haplotypes, as well as individual polymorphisms, on chromosome 10q and AD as provided in Table 13.

With respect to the results shown in Table 13, the IDE 4-locus haplotype (IDE Haplotype 1) provided the strongest haplotypic evidence for association. This global association is mainly driven by the third most frequent haplotype (IDE Haplotype 1a; frequency=8.4% as compared to frequencies of 6.9%, 17.1% and 66% for IDE Haplotypes 1b, 1c and 1d, respectively), which is associated with risk for AD. Two of the haplotypes (Haplotypes 1b and 1c) showed a trend or slight trend toward association with protection against AD, whereas Haplotype 2d showed a slight trend toward association with risk for AD.

The IDE polymorphism located at a position corresponding to position 122260 of SEQ ID NO:484 was found to be significantly associated with risk for AD as an individual SNP. The significant association of this polymorphism with AD was confirmed in an association analysis of a 5-locus haplotype that includes this polymorphism as well as four additional polymorphisms located at positions corresponding to the following positions in SEQ ID NO:484: 120416, 120288, 80752 and 54795. One haplotype of these 5 polymorphisms, in which the nucleotides at positions corresponding to positions 122260, 120416, 120288, 80752 and 54795 of SEQ ID NO:484 are G, A, G, A and G, respectively, revealed an association with risk for AD. Another haplotype of these 5 polymorphisms revealed an association with protection against AD. The nucleotides in this second haplotype of 5 polymorphisms are the same as stated for the first haplotype of these 5 polymorphisms, except for the nucleotide at position 122260 of SEQ ID NO:484, which is an A in the second haplotype. The fact that the only difference in the alleles of the these two haplotypes of 5 polymorphisms is the allele at position 122260 of SEQ ID NO:484 clearly demonstrates that the polymorphism at this position drives the association of the haplotypes with AD. Thus, in essence, the haplotypes of these 5 polymorphisms can be viewed as a genotypic association embedded within a haplotype. Therefore, the association of these 5-SNP haplotypes, which completely depends on the single nucleotide polymorphism located at a position corresponding to position 122260 of SEQ ID NO:484, confirms the significant association of this SNP with AD. The location of this polymorphism at a position upstream of a transcription initiation site(s) (1,966 nucleotides upstream of IDE exon 1 as well as upstream of the KNSL1 gene transcription initiation site) indicates that it is in a region that may be involved in regulation of gene transcription, for example, regulation of transcription of one or both of the IDE and KNSL1 genes.

The global association of KNSL1 Haplotype 1 was mainly attributed to the least frequent haplotype (KNSL1 Haplotype 1a; frequency=17.9% as compared to frequencies of 35.6% and 43% for KNSL1 Haplotypes 1b and 1c, respectively). The strongest association with risk for AD of the individual KNSL1 polymorphisms was the insertion polymorphism located between positions corresponding to positions 178980 and 178981 of SEQ ID NO:484. Similar to the polymorphisms in Table 14 herein that are in linkage disequilibrium with SNP 122260 of SEQ ID NO:484, the polymorphisms that are in linkage disequilibrium with position 178980/178981 of SEQ ID NO:484 (a position associated with AD) are also associated with AD, and may therefore be assessed in place of assessing the polymorphism at position 178980/178981 of SEQ ID NO:484 for each of the methods and applications provided herein that assess the polymorphism at position 178980/178981 of SEQ ID NO:484. This polymorphism was found to be in tight linkage disequilibrium with the two other individually-associating polymorphisms of KNSL1 listed in Table 13 (i.e., 7-bp polyT insertion between positions corresponding to positions 133354 and 133355 of SEQ ID NO:484 and the “A” allele at a position corresponding to position 132370 of SEQ ID NO:484). Similar to the polymorphisms in Table 14 herein that are in linkage disequilibrium with SNP 122260 of SEQ ID NO:484, the polymorphisms that are in linkage disequilibrium with position 133354/133355 of SEQ ID NO:484 (a position associated with AD) are also associated with AD, and may therefore be assessed in place of assessing the polymorphism at position 133354/133355 of SEQ ID NO:484 for each of the methods and applications provided herein that assess the polymorphism at position 133354/133355 of SEQ ID NO:484. Likewise, the polymorphisms that are in linkage disequilibrium with position 132370 of SEQ ID NO:484 (a position associated with AD) are also associated with AD, and may be therefore be assessed in place of assessing the polymorphism at position 132370 of SEQ ID NO:484 for each of the methods and applications provided herein that assess the polymorphism at position 132370 of SEQ ID NO:484.

To investigate whether the underlying association signal in the IDE and KNSL1 genes (neighboring genes on human chromosome 10) originates from the IDE gene, the KNSL1 gene or the ˜20 kb interval in between the 5′ ends of the two genes, a systematic screening for association of 3- and 4-locus haplotypes across the IDE and KNSL1 gene regions was conducted. The results of these analyses confirmed the strong evidence for association and indicated the presence of an AD predisposing/modifying variant in the area of the IDE gene region with polymorphisms in the KNSL1 region being in linkage disequilibrium with such a variant. This is supported by strong linkage disequilibrium observed in the sample DNA across the entire ˜100 kb region on human chromosome 10q23-24 between the IDE polymorphism located at a position corresponding to position 80752 of SEQ ID NO:484 and the insertion polymorphism located between positions corresponding to positions 178980 and 178981 of SEQ ID NO:484.

TABLE 13
ASSOCIATION ANALYSES

| Gene Designation | Polymorphism Position Number (nucleotide) | Association^{a} |
|---|---|---|
| IDE Haplotype 1a | 1. 121239 of SEQ ID NO: 484 (C) | Significant association |
| | 2. 120416 of SEQ ID NO: 484 (A) | with risk for AD |
| | 3. 120288 of SEQ ID NO: 484 (A) | |
| | 4. 80752 of SEQ ID NO: 484 (A) | |
| IDE Haplotype 1b | 1. 121239 of SEQ ID NO: 484 (A) | Trend toward |
| | 2. 120416 of SEQ ID NO: 484 (A) | association with |
| | 3. 120288 of SEQ ID NO: 484 (G) | protection against for |
| | 4. 80752 of SEQ ID NO: 484 (G) | AD |
| IDE Haplotype 1c | 1. 121239 of SEQ ID NO: 484 (A) | Slight trend toward |
| | 2. 120416 of SEQ ID NO: 484 (G) | association with |
| | 3. 120288 of SEQ ID NO: 484 (G) | protection against AD |
| | 4. 80752 of SEQ ID NO: 484 (G) | |
| IDE Haplotype 1d | 1. 121239 of SEQ ID NO: 484 (A) | Slight trend toward |
| | 2. 120416 of SEQ ID NO: 484 (A) | association with risk |
| | 3. 120288 of SEQ ID NO: 484 (G) | for AD |
| | 4. 80752 of SEQ ID NO: 484 (A) | |
| IDE Haplotype 2a | 1. 121239 of SEQ ID NO: 484 (C) | Significant association |
| | 2. 120416 of SEQ ID NO: 484 (A) | with risk for AD |
| | 3. 80752 of SEQ ID NO: 484 (A) | |
| IDE Haplotype 2b | 1. 120416 of SEQ ID NO: 484 (A) | Significant association |
| | 2. 120288 of SEQ ID NO: 484 (A) | with risk for AD |
| | 3. 80752 of SEQ ID NO: 484 (A) | |
| IDE | 122260 of SEQ ID NO: 484 (G) | Significant association |
| | | with risk for AD |
| KNSL1 Haplotype | 1. 132370 of SEQ ID NO: 484 (A) | Trend toward |
| 1a | 2. 133354/133355 of SEQ ID | association with risk |
| | NO: 484 (insertion of TTTTTTT) | for AD |
| | 3. 147842-147845 of SEQ ID | |
| | NO: 484 (presence of AGTT) | |
| | 4. 178980/178981 of SEQ ID | |
| | NO: 484 (insertion of AATTT) | |
| KNSL1 Haplotype | 1. 132370 of SEQ ID NO: 484 (G) | Slight trend toward |
| 1b | 2. 133354/133355 of SEQ ID | association with |
| | NO: 484 (no polyT insertion) | protection against for |
| | 3. 147842-147845 of SEQ ID | AD |
| | NO: 484 (presence of AGTT) | |
| | 4. 178980/178981 of SEQ ID | |
| | NO: 484 (no insertion of | |
| | AATTT) | |
| KNSL1 Haplotype | 1. 132370 of SEQ ID NO: 484 (A) | Slight trend toward |
| 1c | 2. 133354/133355 of SEQ ID | association with risk |
| | NO: 484 (insertion of TTTTTTT) | for AD |
| | 3. 147842-147845 of SEQ ID | |
| | NO: 484 (AGTT deleted at these | |
| | positions) | |
| | 4. 178980/178981 of SEQ ID | |
| | NO: 484 (insertion of AATTT) | |
| KNSL1 | 178980/178981 of SEQ ID NO: 484 | Significant association |
| | (insertion of AATTT) | with risk for AD |
| KNSL1 | 133354/133355 of SEQ ID NO: 484 | Significant association |
| | (insertion of TTTTTTT) | with risk for AD |
| KNSL1 | 132370 of SEQ ID NO: 484 (A) | Significant association |
| | | with risk for AD |
| LIPA | 1. 1852 (A) | Significant association |
| | 2. 6063 (G) | with protection |
| | 3. 7820 (C) | against AD |

^{a}Association is reported as “significant” if analysis yielded p = .05 in the full NIMH sample set; as a “trend” if analysis yielded .05 < p = .10 in the full NIMH sample set; and as a “slight trend” if analysis yielded .10 < p = .35 in the full NIMH sample set.

### EXAMPLE 6

Linkage Disequilibrium Analysis

As described herein, the single nucleotide polymorphism located at the position corresponding to position 122260 of SEQ ID NO:484 is significantly associated with risk for AD. A subset (approximately 24 individuals with the best evidence of linkage to markers in the chromosome 10 region of interest) of the NIMH Genetics Initiative sample set was analyzed to assess whether there was linkage disequilibrium between position 122260 and any other polymorphic positions. There are a number of methods known in the art for making a direct determination of linkage disequilibrium between two or more polymorphic regions. In one method, two or more genetic positions are compared within a population to assess whether the occurrence of one specific allele at one locus consistently correlates with the occurrence of a specific allele at a second locus. This can be done, for example, by direct sequence analysis of genomic DNA and comparison of the nucleotide(s) identified at the same pair of polymorphic sites in each person in order to detect a pattern of non-random association. The extent of linkage disequilibrium can be assessed, for example, by calculation of the standardized multiallelic disequilibrium coefficient D′ [Hedrick (1987) Genetics 117:331-341; Devlin and Risch (1995) Genomics 29:311-3221.

Genomic DNA from the subset of 24 individuals of the NIMH sample was sequenced, and the identities of the nucleotides at two polymorphic sites (a position corresponding to position 122260 of SEQ ID NO:424 and a series of second sites) were compared between the individuals to detect any correlations between the individuals. From this analysis it was observed that there was perfect linkage disequilibrium between the allele at the position corresponding to position 122260 of SEQ ID NO:484 and each of several positions corresponding to positions within SEQ ID NO:484 set forth in Table 14.

Specifically, if the allele present in SEQ ID NO:484 is designated the “reference allele” and the allele that corresponds to a change relative to SEQ ID NO:484 is designated the “non-reference allele,” for each sample observed to be homozygous for the reference allele at position 122260 (i.e. A), that sample was also homozygous for the reference allele at each of the positions set forth in Table 14. Likewise, each sample observed to be homozygous for the non-reference allele at position 122260 (i.e. G) was also homozygous for the non-reference allele at each of the positions set forth in Table 14. Similarly, each sample observed to be heterozygous at position 122260 (i.e. one copy of the A allele and one copy of the G allele) was also heterozygous at each of the positions set forth in Table 14.

Because position 122260 of SEQ ID NO:484 is in linkage disequilibrium (LD) with each of the positions set forth in Table 14, the polymorphisms at each of these positions may be assessed in place of assessing the polymorphism at position 122260 for all applications described herein for the polymorphism at position 122260. Each of the polymorphisms listed in Table 14 (as well as any polymorphism in linkage disequilibrium with the polymorphism at position 122260 of SEQ ID NO:484, or in linkage disequilibrium with any polymorphism described herein as being associated with either risk for or protection against diseases, such as AD) is associated with neurodegenerative disease, in particular, AD. The identity of the particular allele or polymorphism at each of the positions listed in Table 14 that associates with neurodegenerative disease can be determined using methods described herein and/or known in the art (e.g., family-based tests for association) for assessing genetic association of a polymorphism with a disease trait.

Thus, each of the polymorphisms listed in Table 14 may be assessed in methods of assessing an individual's level of risk for developing a neurodegenerative disease or for determining the occurrence of a neurodegenerative disease in an individual, as described herein, for example, with respect to methods in which the polymorphism at position 122260 is assessed.

TABLE 14

| | | NUCLEOTIDE |
|---|---|---|
| | | CHANGE RELATIVE |
| NAME OF | POSITION IN | TO SEQ ID NO: 484^{a} |
| POLYMORPHISM IN | SEQ ID | (reference→non- |
| LD WITH IDE_us_1966 | NO: 484 | reference) |
| (IDE_us_1966) | (122260) | (A→G) |
| IDE_1i.D + 11706 | 108434 | C→G |
| IDE_1i.D + 13145 | 106995 | C→T |
| IDE_1i.D + 21864 | 98276 | G→A |
| IDE_1i.D + 22770 | 97370 | C→T |
| IDE_1i.D + 8168 | 111972 | C→T |
| IDE_1i.D + 9270 | 110870 | C→T |
| IDE_us-3130 | 123424 | G→A |
| IDE_us-4398 | 124692 | A→G |
| KNSL1_us-8576 | 130876 | C→T |
| KNSL1_us-7764 | 131688 | T→G |
| KNSL1_us-5422 | 134030 | G→A |

^{a}For IDE, this is antisense strand; for KNSL1, this is sense strand

Since modifications will be apparent to those of skill in the art, it is intended this invention be limited only by the scope of the appended claims.

### SUMMARY OF SEQUENCE LISTING

SEQ ID NOs:1-71 correspond to both amplification and sequencing primers for the SNCG gene-as set forth in Table 1.

SEQ ID NO:72 is human genomic DNA corresponding to GenBank Accession No. AF037207.1 having the SNCG gene (a.k.a. human persyn) therein plus an additional 175 nucleotides of 3′ flanking sequence corresponding to the reverse complement of nucleotides 235901-236075 of GenBank Accession No. AC025039.4.

SEQ ID NO:73 is identical to SEQ ID NO:72, except that polymorphic regions are indicated throughout.

SEQ ID NOs:74-185 correspond to both amplification and sequencing primers for the IDE gene as set forth in Table 3.

SEQ ID NO:186 is human genomic DNA corresponding to the reverse complement of NCBI Accession No. AL356128.15 having the IDE gene therein.

SEQ ID NO:187 is identical to SEQ ID NO:186, except that polymorphic regions are indicated throughout.

SEQ ID NOs:188-346 correspond to both amplification and sequencing primers for the KNSL1 gene as set forth in Table 5.

SEQ ID NO:347 is human genomic DNA corresponding to the reverse complement of a 63,834 nucleotide portion of NCBI Accession No. NT_{—}008769.1 starting at nucleotide 1,669,312 and ending at nucleotide 1,733,136, having the KNSL1 gene therein.

SEQ ID NO:348 is identical to SEQ ID NO:347, except that polymorphic regions are indicated throughout.

SEQ ID NOs:349-401 correspond to both amplification and sequencing primers for the TNFRSF6 gene as set forth in Table 7.

SEQ ID NO:402 is human genomic DNA corresponding to the reverse complement of a 28,118 nucleotide portion of NCBI Accession No. AL157394.11 starting at nucleotide 17,215 and ending at nucleotide 45,332, having the TNFRSF6 gene therein.

SEQ ID NO:403 is identical to SEQ ID NO:402, except that polymorphic regions are indicated throughout.

SEQ ID NOs:404-466 correspond to both amplification and sequencing primers for the LIPA gene as set forth in Table 9.

SEQ ID NO:467 is human genomic DNA corresponding to the 40,178 nucleotide portion of NCBI Accession No. NT_{—}008679.5 starting at nucleotide 6,017,146 and ending at 6,057,323, having the LIPA gene therein.

SEQ ID NO:468 is identical to SEQ ID NO:467, except that polymorphic regions are indicated throughout.

SEQ ID NO:469 corresponds to a cDNA provided herein encoding a human SNCG protein.

SEQ ID NO:470 corresponds to a cDNA provided herein encoding a human IDE protein.

SEQ ID NOs:471, 473 and 475 correspond to cDNAs provided herein encoding human KNSL1 proteins.

SEQ ID NOs:472, 474 and 476 correspond to the human KNSL1 proteins provided herein.

SEQ ID NOs:477-481 correspond to cDNAs provided herein encoding a human TNFRSF6 protein.

SEQ ID NO:482 corresponds to a cDNA provided herein encoding a human LIPA protein.

SEQ ID NO:483 corresponds to the human SNCG genomic sequence (GenBank accession AF044311) including an additional 909 nucleotides of 5′ flanking sequence obtained from a BLAST search of the NCBI human EST database.

SEQ ID NO:484 corresponds to the genomic DNA sequence corresponding to the IDE/KNSL1 genes taken from hg12 chromosome build 10:93094801 to 93296900 (see also FIG. 6) available from “www.genome.ucsc.edu”.

SEQ ID NOs:485-508 correspond to primers used for testing the particular IDE and KNSL1 polymorphic regions as set forth in Example 3 and FIG. 7.

SEQ ID NOs:509-558 correspond to amplification and sequencing primers for the uPA gene as set forth in Table 11.

SEQ ID NO:559 is human genomic DNA sequence corresponding to nucleotides 827 to 9141 of Genbank Accession No. AF377330 having the uPA gene therein. The sequence shown is that of the sense strand of the genomic DNA (see FIG. 8).

SEQ ID NO:560 is SEQ ID N0:559, except that polymorphic regions are indicated throughout.

SEQ ID NO:561 is a cDNA sequence corresponding to Genbank Accession No. NM_{—}002658 encoding a human uPA protein. Polymorphic regions are indicated throughout (see FIG. 9).

SEQ ID NO:562 is an amino acid sequence for a human uPA protein.

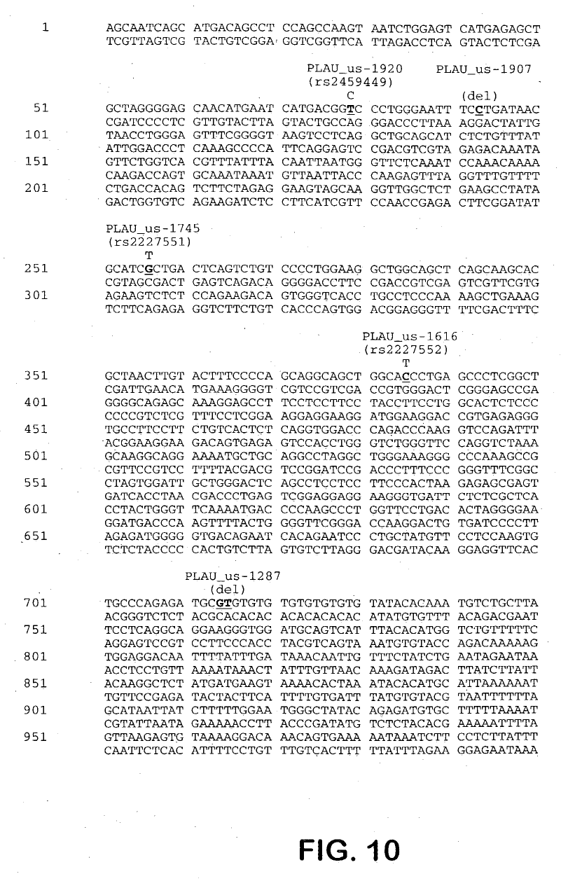

SEQ ID NO:563 is the reverse complement of the nucleotides 74623356-74624256 on Chromosome 10 from the Human Genome Draft build hg12 (see FIG. 10), which is available at “www.genome.ucsc.edu”.

SEQ ID NO:564 is a human genomic DNA sequence corresponding to Genbank Accession No. AF377330 having the uPA gene therein. The polymorphism as position 82 in SEQ ID NO:564 corresponds to the polymorphism at position 256 in SEQ ID NO:563 and Table 12-B.

Since modifications will be apparent to those of skill in the art, it is intended this invention be limited only by the scope of the appended claims.

1. An isolated nucleic acid molecule, comprising at least 14 contiguous nucleotides of an IDE gene allele corresponding to a sequence of 14 contiguous nucleotides that includes nucleotide position 122260 of SEQ ID NO:484, or the complement thereof, except that the nucleotide at position 122260 is replaced with a G, T or C, or is replaced with a C, A or G in a complementary sequence thereof.

2. The nucleic acid of claim 1, wherein the 14 contiguous nucleotides comprise a sequence of 5 contiguous nucleotides of SEQ ID NO:484, or the complement thereof, within the sequence of nucleotides from position 122256 to 122264, except that the nucleotide at position 122260 is replaced with a G, T or C, or is replaced with a C, A or G in a complementary sequence thereof.

3. The nucleic acid of claim 2, wherein the sequence of at least 14 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in a complementary sequence thereof.

4. The nucleic acid of claim 1, wherein the nucleotide at position 122260 is replaced with a G, or is replaced with a C in a complementary position thereof.

5. The nucleic acid molecule of claim 1, further comprising a coding nucleotide sequence operatively linked to a promoter.

6. An isolated nucleic acid molecule, comprising a coding nucleotide sequence operatively linked to a promoter, wherein the coding nucleotide sequence is contained within a nucleotide sequence comprising a portion of an IDE gene allele that comprises a sequence of at least 14 contiguous nucleotides of an IDE gene allele but does not contain a contiguous sequence of a complete IDE gene allele,
wherein the sequence of at least 14 contiguous nucleotides corresponds to a sequence of 14 contiguous nucleotides that includes nucleotide position 122260 of SEQ ID NO:484, or the complement thereof, wherein the nucleotide at position 122260 is A, T, C or G; and
wherein the isolated nucleic acid includes sequence that is heterologous to the IDE gene allele.

7. The isolated nucleic acid molecule of claim 6, wherein the sequence of at least 14 contiguous nucleotides comprises a sequence of 5 contiguous nucleotides of SEQ ID NO:484, or the complement thereof, within the sequence of nucleotides from position 122256 to 122264, wherein the nucleotide at position 122260 is A, T, C or G.

8. The nucleic acid molecule of claim 7, wherein the sequence of at least 14 contiguous nucleotides comprises a sequence of at least 14 contiguous nucleotides of SEQ ID NO:484, or the complement thereof, that comprises said nucleotide at position 122260.

9. The nucleic acid molecule of claim 6, wherein the coding nucleotide sequence encodes a reporter molecule that is not an IDE protein.

10. The nucleic acid molecule of claim 6, wherein the coding nucleotide sequence encodes an IDE protein.

11. The nucleic acid molecule of claim 6, wherein the promoter comprises a promoter that is heterologous to the IDE gene.

12. The nucleic acid molecule of claim 6, wherein the promoter comprises an IDE gene promoter.

13. A vector comprising the nucleic acid molecule of claim 1.

14. A cell comprising the nucleic acid molecule of claim 1, wherein the nucleic acid molecule is heterologous to the cell.

15. A non-human transgenic animal, comprising the nucleic acid molecule of claim 1, wherein the nucleic acid molecule is a transgenic element of the animal.

16. An isolated nucleic acid molecule, comprising at least 14 contiguous nucleotides of a KNSL1 gene allele corresponding to a sequence of 14 contiguous nucleotides that includes nucleotide position 41014 and/or 41015 of SEQ ID NO:347, or the complement thereof, wherein the sequence of 14 contiguous nucleotides comprises one or more nucleotides inserted between positions 41014 and 41015, or the complementary positions thereof.

17. The nucleic acid molecule of claim 16, wherein the 14 contiguous nucleotides comprise the nucleotide sequence AATTT, or the complement thereof, inserted between positions 41014 and 41015, or the complementary positions thereof.

18. The isolated nucleic acid molecule of claim 17, wherein the 14 contiguous nucleotides comprise a sequence of 5 contiguous nucleotides of SEQ ID NO: 347, or the complement thereof, within the sequence of nucleotides from position 41011 to 41018, except that between the nucleotides at positions 41014 and 41015 the sequence AATTT is inserted, or the complement thereof is inserted in the complementary sequence.

19. The nucleic acid molecule of claim 18, wherein the sequence of at least 14 contiguous nucleotides comprises a sequence of at least 14 contiguous nucleotides of SEQ ID NO:347, or the complement thereof, that comprises said nucleotide position 41014 and/or 41015 and the nucleotide sequence AATTT inserted between nucleotide positions 41014 and 41015.

20. The nucleic acid molecule of claim 17, further comprising a coding nucleotide sequence operatively linked to a promoter.

21. A vector comprising the nucleic acid molecule of claim 17.

22. A cell comprising the nucleic acid molecule of claim 17, wherein the nucleic acid molecule is heterologous to the cell.

23. A non-human transgenic animal, comprising the nucleic acid molecule of claim 17, wherein the nucleic acid molecule is a transgenic element of the animal.

24. An isolated nucleic acid molecule, comprising a coding nucleotide sequence operatively linked to a promoter, wherein the coding nucleotide sequence is contained within a nucleotide sequence comprising a portion of a KNSLL gene allele that comprises a sequence of at least 14 contiguous nucleotides of a KNSL1 gene allele but does not contain a contiguous sequence of a complete KNSL1 gene allele,
wherein the sequence of at least 14 contiguous nucleotides corresponds to a sequence of 14 contiguous nucleotides that includes nucleotide position 41014 and/or 41015 of SEQ ID NO:347, or the complement thereof, wherein the sequence does or does not contain one or more nucleotides inserted between nucleotide positions 41014 and 41015, or the complementary positions thereof.

25. The nucleic acid molecule of claim 24, wherein the sequence of at least 14 contiguous nucleotides that includes nucleotide position 41014 and/or 41015 of SEQ ID NO:347, or the complement thereof, does or does not contain the nucleotide sequence AATTT, or the complement thereof, inserted between nucleotide positions 41014 and 41015, or the complementary positions thereof.

26. The nucleic acid molecule of claim 24, wherein the sequence of at least 14 contiguous nucleotides comprises a sequence of 5 contiguous nucleotides of SEQ ID NO:347, or the complement thereof, within the sequence of nucleotides from position 41010 to 41019, wherein the sequence does or does not contain one or more nucleotides inserted between nucleotide positions 41014 and 41015, or the complementary positions thereof.

27. The nucleic acid molecule of claim 26, wherein the sequence of at least 14 contiguous nucleotides comprises a sequence of at least 14 contiguous nucleotides of SEQ ID NQ:347, or the complement thereof, wherein the sequence does or does not contain one or more nucleotides inserted between nucleotide positions 41014 and 41015, or the complementary positions thereof.

28. The nucleic acid molecule of claim 24, wherein the coding nucleotide sequence encodes a reporter molecule that is not a KNSL1 protein.

29. The nucleic acid molecule of claim 24, wherein the coding nucleotide sequence encodes a KNSL1 protein.

30. The nucleic acid molecule of claim 24, wherein the promoter comprises a promoter that is heterologous to a KNSL1 gene.

31. The nucleic acid molecule of claim 24, wherein the promoter comprises a KNSL1 gene promoter.

32. An isolated nucleic acid molecule, comprising at least 50 contiguous nucleotides of a KNSL1 gene allele corresponding to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof.

33. The nucleic acid of claim 32, wherein the sequence of at least 50 contiguous nucleotides comprises a 6-, 7- or 8-bp poly-T sequence, or the complement thereof, inserted between nucleotides at positions 133354 and 133355, or the complementary positions thereof.

34. The isolated nucleic acid molecule of claim 33, wherein the 50 contiguous nucleotides comprise a sequence of 5 contiguous nucleotides of SEQ ID NO: 484, or the complement thereof, within the ,sequence of nucleotides from position 133351 to 133358, except that between the nucleotides at positions 133354 and 133355 a 6-, 7-, or 8- bp polyT sequence is inserted, or the complement thereof is inserted in the complementary sequence.

35. The nucleic acid molecule of claim 34, wherein the sequence of at least 50 contiguous nucleotides comprises a sequence of at least 50 contiguous nucleotides of SEQ ID NO:484, or the complement thereof, that comprises said nucleotide position 133354 and/or 133355 and a 6-, 7-, or 8-bp polyT nucleotide sequence inserted between nucleotide positions 133354 and 133355.

36. The nucleic acid molecule of claim 33, wherein a 7-bp polyT nucleotide sequence, or the complement thereof, is inserted between nucleotide positions 133354 and 133355, or the complementary positions thereof.

37. The nucleic acid molecule of claim 33, further comprising a coding nucleotide sequence operatively linked to a promoter.

38. An isolated nucleic acid molecule, comprising a coding nucleotide sequence operatively linked to a promoter, wherein the coding nucleotide sequence is contained within a nucleotide sequence comprising a portion of a KNSL1 gene allele that comprises a sequence of at least 50 contiguous nucleotides of a KNSL1 gene allele but does not contain a contiguous sequence of a complete KNSL1 gene allele,
wherein the sequence of at least 50 contiguous nucleotides corresponds to a sequence of 50 contiguous nucleotides that includes nucleotide position 133354 and/or 133355 of SEQ ID NO:484, or the complement thereof, wherein the sequence does or does not contain one or more nucleotides inserted between nucleotide positions 133354 and 133355; and
wherein the isolated nucleic acid includes sequence that is heterologous to the KNSL1 gene allele.

39. The nucleic acid molecule of claim 38, wherein the sequence of 50 contiguous nucleotides that includes nucleotide position 133354 and/or 133355 of SEQ ID NO:484, or the complement thereof, does or does not contain a 6-, 7- or 8-bp polyT sequence inserted between nucleotide positions 133354 and 133355.

40. The isolated nucleic acid molecule of claim 38, wherein the sequence of at least 50 contiguous nucleotides comprises a sequence of 5 contiguous nucleotides of SEQ ID NO:484, or the complement thereof, within the sequence of nucleotides from position 133350 to 133359, wherein the sequence does or does not contain one or more nucleotides inserted between nucleotide positions 133354 and 133355.

41. The nucleic acid molecule of claim 40, Wherein the sequence of at least 50 contiguous nucleotides comprises a sequence of at least 50 contiguous nucleotides of SEQ ID NO:484, or the complement thereof, that comprises said nucleotide at position 133354 and/or 133355 and does or does not contain one or more nucleotides inserted between nucleotide positions 133354 and 133355.

42. The nucleic acid molecule of claim 38, wherein the coding nucleotide sequence encodes a reporter molecule that is not a KNSL1 protein.

43. The nucleic acid molecule of claim 38, wherein the coding nucleotide sequence encodes a KNSL1 protein.

44. The nucleic acid molecule of claim 38, wherein the promoter comprises a promoter that is heterologous to a KNSL1 gene.

45. The nucleic acid molecule of claim 38, wherein the promoter comprises a KNSL1 gene promoter.

46. A vector comprising the nucleic acid molecule of claim 33.

47. A cell comprising the nucleic acid molecule of claim 33, wherein the nucleic acid molecule is heterologous to the cell.

48. A non-human transgenic animal, comprising the nucleic acid molecule of claim 33, wherein the nucleic acid molecule is a transgenic element of the animal.

49. An isolated nucleic acid molecule, comprising at least 50 contiguous nucleotides of a KNSL1 gene allele corresponding to a sequence of at least 50 contiguous nucleotides that includes nucleotide position 133354 of SEQ ID NO:484, or the complement thereof, except that the nucleotide at position 133354, or the complementary position thereof, is deleted.

50. The nucleic acid molecule of claim 49, wherein the 50 contiguous nucleotides comprise a sequence of 5 contiguous nucleotides of SEQ ID NO:484, or the complement thereof, within the sequence of nucleotides from position 133351 to 133357, except that the nucleotide at position 133354, or the complementary position thereof, is deleted.

51. The nucleic acid molecule of claim 50, wherein the sequence of at least 50 contiguous nucleotides comprises a sequence of at least 50 contiguous nucleotides of SEQ ID NO: 484, or the complement thereof, except that the nucleotide at position 133354, or the complementary position thereof, is deleted.

52. An isolated nucleic acid molecule, comprising a coding nucleotide sequence operatively linked to a promoter, wherein the coding nucleotide sequence is contained within a nucleotide sequence comprising a portion of a KNS-1 gene allele that comprises a sequence of at least 14 contiguous nucleotides of a KNSL1 gene allele but does not contain a contiguous sequence of a complete KNSL1 gene allele,
wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, T, C or G; and
wherein the isolated nucleic acid includes sequence that is heterologous to the KNSL1 gene allele.

53. The isolated nucleic acid molecule of claim 52, wherein the sequence of at least 14 contiguous nucleotides comprises a sequence of 5 contiguous nucleotides of SEQ ID NO:484, or the complement thereof, within the sequence of nucleotides from position 132366 to 132374, wherein the nucleotide at position 132370 is A, T, C or G.

54. The nucleic acid molecule of claim 53, wherein the sequence of at least 14 contiguous nucleotides comprises a sequence of at least 14 contiguous nucleotides of SEQ ID NO:484, or the complement thereof, that comprises said nucleotide at position 132370.

55. The nucleic acid molecule of claim 52, wherein the nucleotide at position 132370 is A, or is T in the complementary position thereof.

56. The nucleic acid molecule of claim 52, wherein the coding nucleotide sequence encodes a reporter molecule that is not a KNSL1 protein.

57. The nucleic acid molecule of claim 52, wherein the coding nucleotide sequence encodes a KNSL1 protein.

58. The nucleic acid molecule of claim 52, wherein the promoter comprises a promoter that is heterologous to the KNSL1 gene.

59. The nucleic acid molecule of claim 52, wherein the promoter comprises a KNSL1 gene promoter.

60. A vector comprising the nucleic acid molecule of claim 52.

61. A cell comprising the nucleic acid molecule of claim 52, wherein the nucleic acid molecule is heterologous to the cell.

62. A non-human transgenic animal, comprising the nucleic acid molecule of claim 52, wherein the nucleic acid molecule is a transgenic element of the animal.

63. A primer, probe or antisense nucleic acid molecule, comprising a sequence of nucleotides that specifically hybridizes adjacent to, or at, a polymorphic region of a KNSL1 or an IDE gene allele corresponding to:
(a) a region that includes position 41014 and/or 41015 of SEQ ID NO:347, or the complementary positions thereof, of a KNSL1 gene allele, or
(b) a region that includes position 133354 and/or 133355 of SEQ ID NO:484, or the complementary positions thereof, of a KNSL1 gene allele, or
(c) a region that includes position 122260 of SEQ ID NO:484, or the complementary position thereof, of an IDE gene allele.

64. The primer, probe or antisense nucleic acid molecule of claim 63, wherein the sequence of nucleotides specifically hybridizes adjacent to, or at, a sequence of nucleotides, or the complement thereof, of SEQ ID NO:347.

65. The primer, probe or antisense nucleic acid molecule of claim 64, wherein the sequence of nucleotides specifically hybridizes adjacent to, or at, a sequence of nucleotides, or the complement thereof, that includes the nucleotide at position 41014 and/or 41015 of SEQ ID NO:347 and contains the nucleotide sequence AATTT, or the complement thereof, inserted between positions 41014 and 41015, or the complementary positions thereof.

66. The primer, probe or antisense nucleic acid molecule of claim 63, wherein the sequence of nucleotides specifically hybridizes adjacent to, or at, a sequence of nucleotides, or the complement thereof, comprising a sequence of 5 contiguous nucleotides of SEQ ID NO:347, within the sequence of nucleotides from position 41011 to 41018, wherein the sequence contains the sequence AATTT, or the complement thereof, inserted between the nucleotides at positions 41014 and 41015, or the complementary positions thereof.

67. The primer, probe or antisense nucleic acid molecule of claim 66, wherein the sequence of nucleotides contains at least 14 nucleotides but less than 1000 nucleotides.

68. The primer, probe or antisense nucleic acid molecule of claim 63, wherein the sequence of nucleotides specifically hybridizes adjacent to, or at, a sequence of nucleotides, or the complement thereof, of SEQ ID NO:484.

69. The primer, probe or antisense nucleic acid molecule of claim 68, wherein the sequence of nucleotides specifically hybridizes adjacent to, or at, a sequence of nucleotides, or the complement thereof, that includes the nucleotide at position 133354 and/or 133355 of SEQ ID NO:484 and does or does not contain a 6-, 7- or 8-bp polyT sequence, or the complement thereof, inserted between positions 133354 and 133355, or the complementary positions thereof.

70. The primer, probe or antisense nucleic acid molecule of claim 63, wherein the sequence of nucleotides specifically hybridizes adjacent to, or at, a sequence of nucleotides, or the complement thereof, comprising a sequence of 5 contiguous nucleotides of SEQ ID NO:484, within the sequence of nucleotides from position 133351 to 133359, wherein the sequence does or does not contain a 6-, 7- or 8-bp polyT sequence, or the complement thereof, inserted between the nucleotides at positions 133354 and 133355, or the complementary positions thereof.

71. The primer, probe or antisense nucleic acid molecule of claim 70, wherein the sequence of nucleotides contains at least 14 nucleotides but less than 1000 nucleotides.

72. The primer, probe or antisense nucleic acid molecule of claim 63, wherein the sequence of nucleotides specifically hybridizes adjacent to, or at, a sequence of nucleotides, or the complement thereof, of SEQ ID NO:484.

73. The primer, probe or antisense nucleic acid molecule of claim 72, wherein the sequence of nucleotides specifically hybridizes adjacent to, or at, a sequence of nucleotides, or the complement thereof, that includes a nucleotide at position 122260 of SEQ ID NO:484 wherein the nucleotide is an A or G, or is a T or C in the complementary sequence thereof.

74. The primer, probe or antisense nucleic acid molecule of claim 63, wherein the sequence of nucleotides specifically hybridizes adjacent to, or at, a sequence of nucleotides, or the complement thereof, comprising a sequence of 5 contiguous nucleotides of SEQ ID NO:484, within the sequence of nucleotides from position 122256 to 122264, wherein the nucleotide at position 122260 is an A or a G, or is a T or a C in the complementary sequence thereof.

75. The primer, probe or antisense nucleic acid molecule of claim 63, wherein the sequence of nucleotides contains at least 14 nucleotides but less than 1000 nucleotides.

76. A method of detecting the presence or absence of a polymorphism of a KNSL1 gene, comprising determining the presence or absence of:
(a) a nucleotide insertion between nucleotides corresponding to nucleotide positions 41014 and 41015 of SEQ ID NO:347, or the complementary positions thereof, or
(b) a nucleotide insertion between nucleotides corresponding to nucleotide positions 133354 and: 133355 of SEQ ID NO:484, or the complementary positions thereof, or
(c) a deletion of the nucleotide at a position corresponding to nucleotide position 133354 of SEQ ID NO:484, or at the complementary position thereof.

77. The method of claim 76, comprising determining the presence or absence of the nucleotide sequence AATTT, or the complement thereof, inserted between nucleotides corresponding to nucleotide positions 41014 and 41015 of SEQ ID NO:347, or the complementary positions thereof.

78. The method of claim 76, comprising determining the presence or absence of a polyT nucleotide sequence, or the complement thereof, inserted between nucleotides corresponding to nucleotide positions 133354 and 133355 of SEQ ID NO:484, or the complementary positions thereof.

79. The method of claim 78, comprising determining the presence or absence of a 6-, 7-, or 8-bp polyT nucleotide sequence, or the complement thereof, inserted between nucleotides corresponding to nucleotide positions 133354 and 133355 of SEQ ID NO:484, or the complementary positions thereof.

80. A method of detecting the presence or absence of a polymorphism of a gene, comprising determining the identity of a nucleotide at a position corresponding to nucleotide position 132370 of SEQ ID NO:484, or the complement thereof.

81. A method of detecting the presence or absence of a polymorphism of a gene, comprising determining the identity of one or more nucleotides at one or more positions corresponding to nucleotide positions 122260, 121239, 120416, 120288, 80752 and 54795 of SEQ ID NO: 484, or the complementary positions thereof.

82. The method of claim 81, wherein the identity of a nucleotide at a position corresponding to nucleotide position 122260 of SEQ ID NO:484, or the complement thereof, is determined.

83. The method of claim 81, wherein the identity of one or more nucleotides at one or more positions corresponding to nucleotide positions 120416, 120288 and 80752 of SEQ ID NO: 484, or the complementary positions, thereof is (are) determined.

84. The method of claim 83, wherein the identities of the nucleotides at each of the positions corresponding to nucleotide positions 120416, 120288 and 80752 of SEQ ID NO: 484, or the complementary positions, thereof are determined.

85. The method of claim 81, wherein the identity of one or more nucleotides at one or more positions corresponding to nucleotide positions 121239, 120416 and 80752 of SEQ ID NO: 484, or the complementary positions, thereof is (are) determined.

86. The method of claim 85, wherein the identities of the nucleotides at each of the positions corresponding to nucleotide positions 121239, 120416 and 80752 of SEQ ID NO: 484, or the complementary positions, thereof are determined.

87. The method of claim 81, wherein the identity of one or more nucleotides at one or more positions corresponding to nucleotide positions 122260, 120416, 120288, 80752 and 54795 of SEQ ID NO: 484, or the complementary positions thereof, is (are) determined.

88. The method of claim 87, wherein the identities of the nucleotides at each of the positions corresponding to nucleotide positions 122260, 120416, 120288, 80752 and 54795 of SEQ ID NO: 484, or the complementary positions thereof, are determined.

89. The method of claim 80 or claim 81, wherein the identity of the nucleotide(s) is determined in nucleic acid obtained from an individual who has or exhibits a characteristic of a neurodegenerative disease or who has a family member who has a neurodegenerative disease.

90. A method for assessing an individual's level of risk for developing a neurodegenerative disease or for determining the occurrence of a neurodegenerative disease in an individual, comprising:
assessing in a nucleic acid sample obtained from an individual the presence of one or more polymorphisms of chromosome 10 selected from the group consisting of:
(a) a nucleotide insertion between nucleotides corresponding to nucleotide positions 41014 and 41015 of SEQ ID NO:347, or the complementary positions thereof;
(b) a nucleotide insertion between nucleotides corresponding to nucleotide positions 133354 and 133355 of SEQ ID NO:484, or the complementary positions thereof; and
(c) a nucleotide at one or more positions corresponding to nucleotide positions 132370, 122260, 121239, 120416, 120288, 80752 and 54795 of SEQ ID NO: 484, or the complementary positions thereof;
wherein the presence of the polymorphism is indicative of risk for or protection against a neurodegenerative disease.

91. The method of claim 90, comprising detecting the presence or absence of a nucleotide insertion between nucleotides corresponding to nucleotide positions 41014 and 41015 of SEQ ID NO:347, or the complementary positions thereof.

92. The method of claim 91, wherein the nucleotide insertion comprises the nucleotide sequence AATTT, or the complement thereof.

93. The method of claim 90, comprising detecting the presence or absence of a nucleotide insertion between nucleotides corresponding to nucleotide positions 133354 and 133355 of SEQ ID NQ:484, or the complementary positions thereof.

94. The method of claim 93, wherein the nucleotide insertion comprises a polyT nucleotide sequence, or the complement thereof.

95. The method of claim 94, wherein the nucleotide insertion is the nucleotide sequence TTTTTTT, or the complement thereof.

96. The method of claim 90, comprising assessing the presence of a polymorphism at one or more positions corresponding to nucleotide positions 132370, 122260, 121239, 120416, 120288, 80752 and 54795 of SEQ ID NO: 484, or the complementary positions thereof.

97. The method of claim 96, comprising assessing the presence of a polymorphism at a position corresponding to nucleotide position 122260 of SEQ ID NO:484, or the complementary position thereof.

98. The method of claim 97, comprising assessing the presence of a G at a position corresponding to nucleotide position 122260 of SEQ ID NO:484, or a C at a position corresponding to the complementary position thereof.

99. The method of claim 97, comprising assessing the presence of an A at a position corresponding to nucleotide position 122260 of SEQ ID NO:484, or a T at a position corresponding to the complementary position thereof.

100. The method of claim 96, comprising assessing the presence of a polymorphism at a position corresponding to nucleotide position 132370 of SEQ ID NO:484, or the complementary position thereof.

101. The method of claim 100, comprising assessing the presence of an A at a position corresponding to nucleotide position 132370 of SEQ ID NO:484, or a T at a position corresponding to the complementary position thereof.

102. The method of claim 96, comprising assessing the presence of one or more of the following nucleotides:
(a) an A at a position corresponding to nucleotide position 121239 of SEQ ID NO:484, or a T at a position corresponding to the complementary position thereof;
(b) an A at a position corresponding to nucleotide position 120416 of SEQ ID NO:484, or a T at a position corresponding to the complementary position thereof;
(c) a G at a position corresponding to nucleotide position 120288 of SEQ ID NO:484, or a C at a position corresponding to the complementary position thereof;
(d) an A at a position corresponding to nucleotide position 80752 of SEQ ID NO:484, or a T at a position corresponding to the complementary position thereof; and
(e) a G at a position corresponding to nucleotide position 54795 of SEQ ID NO:484, or a C at a position corresponding to the complementary position thereof.

103. The method of claim 96, comprising assessing the presence of a polymorphism at each of the positions corresponding to nucleotide positions 120416, 120288 and 80752 of SEQ ID NO: 484, or the complementary positions thereof.

104. The method of claim 103, comprising assessing the presence of an A at a position corresponding to nucleotide position 120416 of SEQ ID NO:484, or a T at a position corresponding to the complementary position thereof; an A at a position corresponding to nucleotide position 120288 of SEQ ID NO:484, or a T at a position corresponding to the complementary position thereof; and an A at a position corresponding to nucleotide position 80752 of SEQ ID NO:484, or a T at a position corresponding to the complementary position thereof.

105. The method of claim 96, comprising assessing the presence of a polymorphism at each of the positions corresponding to nucleotide positions 121239, 120416 and 80752 of SEQ ID NO: 484, or the complementary positions thereof.

106. The method of claim 105, comprising assessing the presence of a C at a position corresponding to nucleotide position 121239 of SEQ ID NO:484, or a G at a position corresponding to the complementary position thereof; an A at a position corresponding to nucleotide position 120416 of SEQ ID NO:484, or a T at a position corresponding to the complementary position thereof; and an A at a position corresponding to nucleotide position 80752 of SEQ ID NO:484, or a T at a position corresponding to the complementary position thereof.

107. The method of claim 96, comprising assessing the presence of a polymorphism at each of the corresponding to nucleotide positions 122260, 120416, 120288, 80752 and 54795 of SEQ ID NO: 484, or the complementary positions thereof.

108. The method of claim 107, comprising assessing the presence of a G at a position corresponding to nucleotide position 122260 of SEQ ID NO:484, or a C at a position corresponding to the complementary position thereof; an A at a position corresponding to nucleotide position 120416 of SEQ ID NO:484, or a T at a position corresponding to the complementary position thereof; a G at a position corresponding to nucleotide position 120288 of SEQ ID NO:484, or a C at a position corresponding to the complementary position thereof; and an A at a position corresponding to nucleotide position 80752 of SEQ ID NO:484, or a T at a position corresponding to the complementary position thereof; and a G at a position corresponding to nucleotide position 54795 of SEQ ID NO:484, or a C at a position corresponding to the complementary position thereof.

109. The method of claim 107, comprising assessing the presence of an A at a position corresponding to nucleotide position 122260 of SEQ ID NO:484, or a T at a position corresponding to the complementary position thereof; an A at a position corresponding to nucleotide position 120416 of SEQ ID NO:484, or a T at a position corresponding to the complementary position thereof; a G at a position corresponding to nucleotide position 120288 of SEQ ID NO:484, or a C at a position corresponding to the complementary position thereof; and an A at a 15 position corresponding to nucleotide position 80752 of SEQ ID NO:484, or a T at a position corresponding to the complementary position thereof; and a G at a position corresponding to nucleotide position 54795 of SEQ ID NO:484, or a C at a position corresponding to the complementary position thereof.

110. The method of any of claims 92, 95, 98, 101, 104, 106 and 108 wherein the presence of the polymorphism is indicative of risk for a neurodegenerative disease.

111. The method of claim 99 or claim 109, wherein the presence of the polymorphism is indicative of protection against a neurodegenerative disease.

112. The method claim 90, wherein the neurodegenerative disease is Alzheimer's disease.

113. The method of claim 90, further comprising determining if the individual is homozygous for the polymorphism.

114. A method of screening for an agent that modulates the expression and/or activity of IDE, comprising:
assessing the effect of a test agent on the expression of a coding nucleotide sequence operatively linked to a promoter, wherein the coding nucleotide sequence is contained within a nucleotide sequence comprising at least a portion of the IDE gene that comprises a sequence of at least 14 contiguous nucleotides of an IDE gene allele, wherein the sequence of at least 14 contiguous nucleotides corresponds to a sequence of at least 14 contiguous nucleotides that includes the nucleotide at position 122260 of SEQ ID NO:484, or the complement thereof, wherein the nucleotide at position 122260 is replaced with a G, T or C, or is replaced with a C, A or G in a complementary sequence thereof; and
identifying a test agent as an agent that modulates the expression and/or activity of IDE if it has an effect on expression of the coding nucleotide sequence.

115. The method of claim 114, wherein the sequence of 14 contiguous nucleotides comprises a sequence of 5 contiguous nucleotides of SEQ ID NO:484, or the complement thereof, within the sequence of nucleotides from position 122256 to 122264, except that the nucleotide at position 122260 is replaced with a G, T or C, or is replaced with a C, A or G in the complement thereof.

116. The method of claim 11 5, wherein the sequence of 14 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in the complement thereof.

117. The method of claim 114, wherein the nucleotide at position 122260 is replaced with a G, or is replaced with a C in a complementary sequence thereof.

118. A method of screening for an agent that modulates the expression and/or activity of IDE, comprising:
assessing the effect of a test agent on the expression of a coding nucleotide sequence operatively linked to a promoter, wherein the coding nucleotide sequence is contained within a nucleotide sequence comprising a portion of the IDE gene that comprises a sequence of at least 14 contiguous nucleotides of an IDE gene allele but that is not a contiguous sequence of a complete IDE allele;
wherein, the sequence of at least 14 contiguous nucleotides corresponds to a sequence of at least 14 contiguous nucleotides that includes the nucleotide at position 122260 of SEQ ID NO:484, or the complement thereof; and
identifying a test agent as an agent that modulates the expression and/or activity of IDE if it has an effect on expression of the coding nucleotide sequence.

119. The method of claim 118, wherein the sequence of at least 14 contiguous nucleotides comprises a sequence of 5 contiguous nucleotides of SEQ ID NO: 484, or the complement thereof, within the sequence of nucleotides from position 122256 to 122264.

120. The method of claim 119, wherein the sequence of 14 contiguous nucleotides comprises a sequence of at least 14 contiguous nucleotides of SEQ ID NO:484, or the complement thereof.

121. The method of claim 118, wherein the coding nucleotide sequence, promoter and portion of the IDE gene are contained in a nucleotide sequence that includes sequence that is heterologous to the IDE gene.

122. The method of claim 114, wherein the coding sequence of nucleotides encodes an IDE protein or a reporter molecule.

123. The method of claim 114, wherein assessing comprises assessing the effect on expression of the coding sequence of nucleotides in a cell that comprises the coding sequence of nucleotides operatively linked to a promoter and contained within a nucleotide sequence comprising the portion of the IDE gene.

124. The method of claim 114, wherein assessing comprises assessing the effect on expression of the coding sequence of nucleotides in a non-human animal that comprises the coding sequence of nucleotides operatively linked to a promoter and contained within a nucleotide sequence comprising the portion of the IDE gene.

125. The method of claim 123, wherein the cell is a recombinant cell.

126. The method of claim 124, wherein the animal is a non-human transgenic animal.

127. The method of claim 123, wherein the coding sequence operatively linked to a promoter and contained within a nucleotide sequence comprising the portion of the IDE gene is heterologous to the cell.

128. The method of claim 124, wherein the coding sequence operatively linked to a promoter and contained within a nucleotide sequence comprising the portion of the IDE gene is a transgenic element in the transgenic animal.

129. The method of claim 114, wherein the promoter comprises an IDE gene promoter.

130. The method of claim 114, wherein a test agent is identified as an agent that modulates the expression and/or activity of IDE if it increases or decreases the level of expression of the coding sequence of nucleotides.

131. The method of claim 114, wherein a test agent is identified as an agent that modulates the expression and/or activity of IDE if it alters the level of expression of the coding sequence of nucleotides such that it is substantially similar to the level of expression of a coding sequence of nucleotides operatively linked to a promoter and contained within a nucleotide sequence comprising the portion of the IDE gene wherein the nucleotide at position 122260 is an A, or wherein the nucleotide at position 122260 is a T in the complementary sequence thereof.

132. The method of claim 114, wherein assessing comprises determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

133. A method of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 50 contiguous nucleotides of a KNSL1 allele,
wherein the sequence of at least 50 contiguous nucleotides corresponds to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof; and
identifying a test agent as an agent that modulates the expression and/or activity of KNSL1 if it has an effect on expression of the coding nucleotide sequence.

134. The method of claim 133, wherein the sequence of at least 50 contiguous nucleotides comprises a 6-, 7- or 8-bp polyT sequence, or the complement thereof, inserted between nucleotides at positions 133354 and 133355, or the complementary positions thereof.

135. The method of claim 133, wherein the sequence of at least 50 contiguous nucleotides comprises a sequence of 5 contiguous nucleotides of SEQ ID NO:484, or the complement thereof, within the sequence of nucleotides from position 133351 to 133358, and one or more nucleotides inserted between positions 133354 and 133355, or the complementary positions thereof.

136. The method of claim 135, wherein the sequence of at least 50 contiguous nucleotides comprises a sequence of at least 50 contiguous nucleotides of SEQ ID NO:484, or the complement thereof, and one or more nucleotides inserted between positions 133354 and 133355, or the complementary positions thereof.

137. A method of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 50 contiguous nucleotides of a KNSL1 gene allele but that is not a contiguous sequence of a complete KNSL1 allele;
wherein the sequence of at least 50 contiguous nucleotides corresponds to a sequence of at least 50 contiguous nucleotides that includes nucleotide position 133354 and/or 133355, or the complement thereof, and contains or does not contain one or more nucleotides inserted between nucleotides 133354 and 133355; and
identifying a test agent as an agent that modulates the expression and/or activity of KNSL1 if it has an effect on expression of the coding nucleotide sequence.

138. The method of claim 1 37, wherein the sequence of at least 50 contiguous nucleotides comprises a sequence of 5 contiguous nucleotides of SEQ ID NO:484, or the complement thereof, within the sequence of nucleotides from position 133350 to 133359, and contains or does not contain one or more nucleotides inserted between nucleotides 133354 and 133355.

139. The method of claim 137, wherein the sequence of at least 50 contiguous nucleotides comprises a sequence of at least 50 contiguous nucleotides of SEQ ID NO:484, or the complement thereof, and contains or does not contain one or more nucleotides inserted between nucleotides 133354 and 133355.

140. The method of claim 137, wherein the coding nucleotide sequence, promoter and portion of the KNSL1 gene are contained in a nucleotide sequence that includes sequence that is heterologous to the KNSL1 gene.

141. The method of claim 133, wherein the coding sequence of nucleotides encodes a KNSL1 protein or a reporter molecule.

142. The method of claim 133, wherein assessing comprises assessing the effect on expression of the coding sequence of nucleotides in a cell that comprises the coding sequence of nucleotides operatively linked to a promoter and contained within a nucleotide sequence comprising the said portion of the KNSL1 gene.

143. The method of claim 133, wherein assessing comprises assessing the effect on expression of the coding sequence of nucleotides in a non-human animal that comprises the coding sequence of nucleotides operatively linked to a promoter and contained within a nucleotide sequence comprising the said portion of the KNSL1 gene.

144. The method of claim 142, wherein the cell is a recombinant cell.

145. The method of claim 143, wherein the animal is a non-human transgenic animal.

146. The method of claim 142, wherein the coding sequence operatively linked to a promoter and contained within a nucleotide sequence comprising the said portion of the KNSL1 gene is heterologous to the cell.

147. The method of claim 143, wherein the coding sequence operatively linked to a promoter and contained within a nucleotide sequence comprising the said portion of the KNSL1 gene is a transgenic element in the transgenic animal.

148. The method of claim 133, wherein the promoter comprises a KNSL1 gene promoter.

149. The method of claim 133, wherein a test agent is identified as an agent that modulates the expression and/or activity of KNSL1 if it increases or decreases the level of expression of the coding sequence of nucleotides.

150. The method of claim 133, wherein a test agent is identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the sequence does not contain one or more nucleotides inserted between nucleotide positions 133354 and 133355.

151. The method of claim 150, wherein the one or more nucleotides inserted between nucleotide positions 133354 and 133355 is a 7-bp polyT sequence, or the complement thereof inserted between the complementary positions thereof.

152. The method of claim 133, wherein assessing comprises determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

153. A method of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 14 contiguous nucleotides of a KNSL1 gene allele,
wherein the sequence of at least 14 contiguous nucleotides corresponds to a sequence of at least 14 contiguous nucleotides that includes nucleotide position 41014 and/or 41015 of SEQ ID NO:347, or the complement thereof, and one or more nucleotides inserted between nucleotides at positions 41014 and 41015, or the complementary positions thereof; and
identifying a test agent as an agent that modulates the expression and/or activity of KNSL1 if it has an effect on expression of the coding nucleotide sequence.

154. The method of claim 153, wherein the sequence of at least 14 contiguous nucleotides comprises the nucleotide sequence AATTT, or the complement thereof, inserted between nucleotides at positions 41014 and 41015, or the complementary positions thereof.

155. The method of claim 153, wherein the sequence of at least 14 contiguous nucleotides comprises a sequence of 5 contiguous nucleotides of SEQ ID NO:347, or the complement thereof, within the sequence of nucleotides from position 41011 to 41018, and one or more nucleotides inserted between positions 41011 and 41018, or the complementary positions thereof.

156. The method of claim 155, wherein the sequence of at least 14 contiguous nucleotides comprises a sequence of at least 14 contiguous nucleotides of SEQ ID NO:347, or the complement thereof, and one or more nucleotides inserted between positions 41011 and 41018, or the complementary positions thereof.

157. A method of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 14 contiguous nucleotides of a KNSL1 gene allele but that is not a contiguous sequence of a complete KNSL1 allele;
wherein the sequence of at least 14 contiguous nucleotides corresponds to a sequence of at least 14 contiguous nucleotides that includes nucleotide position 41014 and/or 41015, or the complement thereof, and contains or does not contain one or more nucleotides inserted between nucleotides 41014 and 41015; and
identifying a test agent as an agent that modulates the expression and/or activity of KNSL1 if it has an effect on expression of the coding nucleotide sequence.

158. The method of claim 157, wherein the sequence of at least 14 contiguous nucleotides comprises a sequence of 5 contiguous nucleotides of SEQ ID NO:347, or the complement thereof, within the sequence of nucleotides from position 41010 to 41019, and contains or does not contain one or more nucleotides inserted between nucleotides 41014 and 41015, or the complementary positions thereof.

159. The method of claim 158, wherein the sequence of at least 14 contiguous nucleotides comprises a sequence of at least 14 contiguous nucleotides of SEQ ID NO:347, or the complement thereof, and contains or does not contain one or more nucleotides inserted between nucleotides 41014 and 41015, or the complementary positions thereof.

160. The method of claim 158, wherein the coding nucleotide sequence, promoter and portion of the KNSL1 gene are contained in a nucleotide sequence that includes sequence that is heterologous to the KNSL1 gene.

161. The method of claim 153, wherein the coding sequence of nucleotides encodes a KNSL1 protein or a reporter molecule.

162. The method of claim 153, wherein assessing comprises assessing the effect on expression of the coding sequence of nucleotides in a cell that comprises the coding sequence of nucleotides operatively linked to a promoter and contained within a nucleotide sequence comprising the portion of the KNSL1 gene.

163. The method of claim 153, wherein assessing comprises assessing the effect on expression of the coding sequence of nucleotides in a non-human animal that comprises the coding sequence of nucleotides operatively linked to a promoter and contained within a nucleotide sequence comprising the portion of the KNSL1 gene.

164. The method of claim 162, wherein the cell is a recombinant cell.

165. The method of claim 163, wherein the animal is a non-human transgenic animal.

166. The method of claim 164, wherein the coding sequence operatively linked to a promoter and contained within a nucleotide sequence comprising the portion of the KNSL1 gene is heterologous to the cell.

167. The method of claim 165, wherein the coding sequence operatively linked to a promoter and contained within a nucleotide sequence comprising the said portion of the KNSL1 gene is a transgenic element in the transgenic animal.

168. The method of claim 153, wherein the promoter comprises a KNSL1 gene promoter.

169. The method of claim 153, wherein a test agent is identified as an agent that modulates the expression and/or activity of KNSL1 if increases or decreases the level of expression of the coding sequence of nucleotides.

170. The method of claim 153, wherein a test agent is identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the sequence does not contain one or more nucleotides inserted between nucleotide positions 41014 and 41015.

171. The method of claim 170, wherein the one or more nucleotides inserted between nucleotide positions 41014 and 41015 comprises the sequence AATTT, or the complement thereof inserted between the complementary positions thereof.

172. The method of claim 153, wherein assessing comprises determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

173. A method of screening for an agent that modulates the expression and/or activity of KNSL1, comprising:
assessing the effect of a test agent on the expression of a coding nucleotide sequence operatively linked to a promoter, wherein the coding nucleotide sequence is contained within a nucleotide sequence comprising at least a portion of the KNSL1 gene that comprises a sequence of at least 14 contiguous nucleotides of a KNSL1 gene allele, wherein, the sequence of at least 14 contiguous nucleotides corresponds to a sequence of 14 contiguous nucleotides that includes nucleotide position 132370 of SEQ ID NO:484, or the complement thereof, except that the nucleotide at position 132370 is replaced with an A or is replaced with a T in the complement thereof; and
identifying a test agent as an agent that modulates the expression and/or activity of KNSL1 if it has an effect on expression of the coding nucleotide sequence.

174. A method of screening for an agent that modulates the expression and/or-activity of KNSL1, comprising:
assessing the effect of a test agent on the expression of a coding nucleotide sequence operatively linked to a promoter, wherein the coding nucleotide sequence is contained within a nucleotide sequence comprising a portion of the KNSL1 gene that comprises a sequence of at least 14 contiguous nucleotides of a KNSL1 gene allele but that is not a contiguous sequence of a complete KNSL1 allele;
wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, G, T or C; and
identifying a test agent as an agent that modulates the expression and/or activity of KNSL1 if it has an effect on expression of the coding nucleotide sequence.

175. The method of claim 173, wherein the sequence of at least 14 contiguous nucleotides comprises a sequence of 5 contiguous nucleotides of SEQ ID NO: 484, or the complement thereof, within the sequence of nucleotides from position 132366 to132374, that comprises said nucleotide at position 132370.

176. The method of claim 173, wherein the sequence of at least 14 contiguous nucleotides is a sequence of at least 14 contiguous nucleotides of SEQ ID NO: 484, or the complement thereof, that comprises said nucleotide position at 132370.

177. The method of claim 174, wherein the coding nucleotide sequence, promoter and portion of the KNSL1 gene are contained in a nucleotide sequence that includes sequence that is heterologous to the KNSL1 gene.

178. The method of claim 173, wherein the coding sequence of nucleotides encodes a KNSL1 protein or a reporter molecule.

179. The method of claim 173, wherein assessing comprises assessing the effect on expression of the coding sequence of nucleotides in a cell that comprises the coding sequence of nucleotides operatively linked to a promoter and contained within a nucleotide sequence comprising the portion of the KNSL1 gene.

180. The method of claim 173, wherein assessing comprises assessing the effect on expression of the coding sequence of nucleotides in a non-human animal that comprises the coding sequence of nucleotides operatively linked to a promoter and contained within a nucleotide sequence comprising the said portion of the KNSL1 gene.

181. The method of claim 179, wherein the cell is a recombinant cell.

182. The method of claim 180, wherein the animal is a non-human transgenic animal.

183. The method of claim 179, wherein the coding sequence operatively linked to a promoter and contained within a nucleotide sequence comprising the said portion of the KNSL1 gene is heterologous to the cell.

184. The method of claim 180, wherein the coding sequence operatively linked to a promoter and contained within a nucleotide sequence comprising the said portion of the KNSL1 gene is a transgenic element in the transgenic animal.

185. The method of claim 173, wherein the promoter comprises a KNSL1 gene promoter.

186. The method of claim 173, wherein a test agent is identified as an agent that modulates the expression and/or activity of KNSL1 if increases or decreases the level of expression of the coding sequence of nucleotides.

187. The method of claim 173, wherein a test agent is identified as an agent that modulates the expression and/or activity of KNSL1 if it alters the level of expression of the coding sequence of nucleotides such that it is substantially similar to the level of expression of a coding sequence of nucleotides operatively linked to a promoter and contained within a nucleotide sequence comprising the said at least a portion of the KNSL1 gene wherein the nucleotide at position 132370 is a G or wherein the nucleotide at position 132370 is a C in the complementary sequence thereof.

188. The method of claim 173, wherein assessing comprises determining the effect on the level of mRNA encoding a protein encoded by the coding sequence of nucleotides or determining the effect on the level of protein or reporter molecule encoded by the coding sequence of nucleotides or determining the effect on the activity of a protein or reporter molecule encoded by the coding sequence of nucleotides.

189. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding IDE operatively linked to a promoter,
wherein the coding nucleotide sequence is contained within a nucleotide sequence comprising at least a portion of the IDE gene that comprises a sequence of at least 14 contiguous nucleotides of an IDE gene allele,
wherein the sequence of at least 14 contiguous nucleotides corresponds to a sequence of at least 14 contiguous nucleotides that includes the nucleotide at position 122260 of SEQ ID NO:484, or the complement thereof, wherein the nucleotide at position 1 22260 is replaced with a G, T or C, or is replaced with a C, A or G in a complementary sequence thereof; and
identifying a test agent as an agent that modulates a biological event characteristic of a neurodegenerative disease if it has an effect on the biological event characteristic of a neurodegenerative disease.

190. The method of claim 189, wherein the sequence of 14 contiguous nucleotides comprises a sequence of 5 contiguous nucleotides of SEQ ID NO:484, or the complement thereof, within the sequence of nucleotides from position 122256 to 122264, except that the nucleotide at position 122260 is replaced with a G, T or C, or is replaced with a C, A or G in the complement thereof.

191. The method of claim 190, wherein the sequence of 14 contiguous nucleotides comprises a sequence of at least 14 contiguous nucleotides of SEQ ID NO:484, or the complement thereof, except that the nucleotide at position 122260 is replaced with a G, T or C, or is replaced with a C, A or G in the complement thereof.

192. The method of claim 1 89, wherein the nucleotide at position 122260 is replaced with a G, or is replaced with a C in a complementary sequence thereof.

193. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding IDE operatively linked to a promoter,
wherein the coding nucleotide sequence is contained within a nucleotide sequence comprising a portion of the IDE gene that comprises a sequence of at least 14 contiguous nucleotides of an IDE gene allele but that is not a contiguous sequence of a complete IDE allele, and
wherein, the sequence of at least 14 contiguous nucleotides corresponds to a sequence of at least 14 contiguous nucleotides that includes the nucleotide at position 122260 of SEQ ID NO:484, or the complement thereof; and
identifying a test agent as an agent that modulates a biological event characteristic of a neurodegenerative disease if it has an effect on the biological event characteristic of a neurodegenerative disease.

194. The method of claim 193, wherein the sequence of 14 contiguous nucleotides comprises a sequence of 5 contiguous nucleotides of SEQ ID NQ:484, or the complement thereof, within the sequence of nucleotides from position 122256 to 122264.

195. The method of claim 194, wherein the sequence of 14 contiguous nucleotides comprises a sequence of at least 14 contiguous nucleotides of SEQ ID NO:484, or the complement thereof.

196. The method of claim 189, wherein the coding nucleotide sequence, promoter and portion of the IDE gene are contained in a nucleotide sequence that includes sequence that is heterologous to the IDE gene.

197. The method of claim 189, wherein the nucleotide sequence comprising a portion of the IDE gene is heterologous to the cell or animal.

198. The method of claim 189, wherein the cell is a recombinant cell.

199. The method of claim 189, wherein the animal is a non-human transgenic animal.

200. The method of claim 189, wherein the promoter comprises an IDE gene promoter.

201. The method of claim 189, wherein the neurodegenerative disease is Alzheimer's disease.

202. The method of claim 189, wherein the biological event is the level of an Aβ peptide in the cell, extracellular medium or animal.

203. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 50 contiguous nucleotides of a KNSL1 gene allele, and
wherein the sequence of at least 50 contiguous nucleotides corresponds to a sequence of at least 50 contiguous nucleotides that includes nucleotide position 133354 and/or 133355 of SEQ ID NO:484, or the complement thereof, and one or more nucleotides inserted between nucleotides at positions 133354 and 133355, or the complementary positions thereof; and
identifying a test agent as an agent that modulates a biological event characteristic of a neurodegenerative disease if it has an effect on the biological event characteristic of a neurodegenerative disease.

204. The method of claim 203, wherein the sequence of at least 50 contiguous nucleotides comprises a 6-, 7- or 8-bp polyT sequence, or the complement thereof, inserted between nucleotides at positions 133354 and 133355, or the complementary positions thereof.

205. The method of claim 203, wherein the sequence of at least 50 contiguous nucleotides comprises a sequence of 5 contiguous nucleotides of SEQ ID NO:484, or the complement thereof, within the sequence of nucleotides from position 133351 to 133358, and one or more nucleotides inserted between positions 133354 and 133355, or the complementary positions thereof.

206. The method of claim 205, wherein the sequence of at least 50 contiguous nucleotides comprises a sequence of at least 50 contiguous nucleotides of SEQ ID NO:484, or the complement thereof, and one or more nucleotides inserted between positions 133354 and 133355, or the complementary positions thereof.

207. The method of claim 203, wherein the sequence of at least 50 contiguous nucleotides comprises a 7-bp polyT sequence, or the complement thereof, inserted between nucleotides at positions 133354 and 133355, or the complementary positions thereof.

208. A method of screening for an agent that modulates a biological event characteristic of a neurodegenerative disease, comprising:
assessing the effect of a test agent on a biological event characteristic-of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 50 contiguous nucleotides of a KNSL1 gene allele but that is not a contiguous sequence of a complete KNSL1 allele, and
wherein the sequence of at least 50 contiguous nucleotides corresponds to a sequence of at least 50 contiguous nucleotides that includes nucleotide position 133354 and/or 133355, or the complement thereof, and contains or does not contain one or more nucleotides inserted between nucleotides 133354 and 133355; and
identifying a test agent as an agent that modulates a biological event characteristic of a neurodegenerative disease if it has an effect on the biological event characteristic of a neurodegenerative disease.

209. The method of claim 208, wherein the sequence of at least contiguous nucleotides comprises a sequence of 5 contiguous nucleotides of SEQ ID NO:484, or the complement thereof, within the sequence of nucleotides from position 133350 to 133359, and contains or does not contain one or more nucleotides inserted between nucleotides 133354 and 133355.

210. The method of claim 209, wherein the sequence of at least 50 contiguous nucleotides comprises a sequence of at least 50 contiguous nucleotides of SEQ ID NO:484, or the complement thereof, and contains or does not contain one or more nucleotides inserted between nucleotides 133354 and 133355.

211. The method of claim 203, wherein the coding nucleotide sequence, promoter and portion of the KNSL1 gene are contained in a nucleotide sequence that includes sequence that is heterologous to the KNSL1 gene.

212. The method of claim 203, wherein the nucleotide sequence comprising a portion of the KNSL1 gene is heterologous to the cell or animal.

213. The method of claim 203, wherein the cell is a recombinant cell.

214. The method of claim 203, wherein the animal is a non-human transgenic animal.

215. The method of claim 203, wherein the promoter comprises a KNSL1 gene promoter.

216. The method of claim 203, wherein the neurodegenerative disease is Alzheimer's disease.

217. The method of claim 203, wherein the biological event is the level of an Aβ peptide in the cell, extracellular medium or animal.

218. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained withina nucleotide sequence comprising at least a portion of the KNSL1 gene that comprises a sequence of at least 14 contiguous nucleotides of a KNSL1 gene allele, and
wherein the sequence of at least 14 contiguous nucleotides corresponds to a sequence of at least 14 contiguous nucleotides that includes nucleotide position 41014 and/or 41015 of SEQ ID NO:347, or the complement thereof, and one or more nucleotides inserted between nucleotides at positions 41014 and 41015, or the complementary positions thereof; and
identifying a test agent as an agent that modulates a biological event characteristic of a neurodegenerative disease if it has an effect on the biological event characteristic of a neurodegenerative disease.

219. The method of claim 218, wherein the sequence of at least 14 contiguous nucleotides comprises the nucleotide sequence AATTT, or the complement thereof, inserted between nucleotides at positions 41014 and 41015, or the complementary positions thereof.

220. The method of claim 218, wherein the sequence of at least 14 contiguous nucleotides comprises a sequence of 5 contiguous nucleotides of SEQ ID NO:347, or the complement thereof, within the sequence of nucleotides from position 41011 to 41018, and one or more nucleotides inserted between positions 41011 and 41018, or the complementary positions thereof.

221. The method of claim 220, wherein the sequence of at least 14 contiguous nucleotides comprises a sequence of at least 14 contiguous nucleotides of SEQ ID NO:347, or the complement thereof, and one or more nucleotides inserted between positions 41011 and 41018, or the complementary positions thereof.

222. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 14 contiguous nucleotides of a KNSL1 gene allele but that is not a contiguous sequence of a complete KNSL1 allele, and
wherein the sequence of at least 14 contiguous nucleotides corresponds to a sequence of at least 14 contiguous nucleotides that includes nucleotide position 41014 and/or 41015, or the complement thereof, and contains or does not contain one or more nucleotides inserted between nucleotides 41014 and 41015; and
identifying a test agent as an agent that modulates a biological event characteristic of a neurodegenerative disease if it has-an effect on the biological event characteristic of a neurodegenerative disease.

223. The method of claim 222, wherein the sequence of at least 14 contiguous nucleotides comprises a sequence of 5 contiguous nucleotides of SEQ ID NO:347, or the complement thereof, within the sequence of nucleotides from position 41010 to 41019, and contains or does not contain one or more nucleotides inserted between nucleotides 41014 and 41015, or the complementary positions thereof.

224. The method of claim 223, wherein the sequence of at least 14 contiguous nucleotides comprises a sequence of at least 14 contiguous nucleotides of SEQ ID NO:347, or the complement thereof, and contains or does not contain one or more nucleotides inserted between nucleotides 41014 and 41015, or the complementary positions thereof.

225. The method of claim 218, wherein the coding nucleotide sequence, promoter and portion of the KNSL1 gene are contained in a nucleotide sequence that includes sequence that is heterologous to the KNSL1 gene.

226. The method of claim 218, wherein the nucleotide sequence comprising a portion of the KNSL1 gene is heterologous to the cell or animal.

227. The method of claim 218, wherein the cell is a recombinant cell.

228. The method of claim 218, wherein the animal is a non-human transgenic animal.

229. The method of 218, wherein the promoter comprises a KNSL1 gene promoter.

230. The method of claim 218, wherein the neurodegenerative disease is Alzheimer's disease.

231. The method of claim 218, wherein the biological event is the level of an Aβ peptide in the cell, extracellular medium or animal.

232. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising at least a portion of the KNSL1 gene that comprises a sequence of at least 14 contiguous nucleotides of a KNSL1 gene allele, and
wherein the sequence of at least 14 contiguous nucleotides corresponds to a sequence of at least 14 contiguous nucleotides that includes nucleotide position 132370 of SEQ ID NO:484, or the complement thereof, except that the nucleotide at position 132370 is replaced with an A or is replaced with a T in the complement thereof; and
identifying a test agent as an agent that modulates a biological event characteristic of a neurodegenerative disease if it has an effect on the biological event characteristic of a neurodegenerative disease.

233. A method of screening for an agent that modulates a biological event characteristic of a neurodegenerative disease, comprising:
assessing the effect of a test agent on a biological event characteristic of a neurodegenerative disease exhibited by a cell or animal that comprises a sequence of nucleotides encoding KNSL1 operatively linked to a promoter,
wherein the coding nucleotide sequence is contained within a nucleotide sequence comprising a portion of the KNSL1 gene that comprises a sequence of at least 14 contiguous nucleotides of a KNSL1 gene allele but that is not a contiguous sequence of a complete KNSL1 allele;
wherein the sequence of at least 14 contiguous nucleotides corresponds to a sequence of 14 contiguous nucleotides that includes nucleotide position 132370 of SEQ ID NO:484, or the complement thereof, wherein the nucleotide at position 132370 is A, G, T or C; and
identifying a test agent as an agent that modulates a biological event characteristic of a neurodegenerative disease if it has an effect on the biological event characteristic of a neurodegenerative disease.

234. The method of claim 232, wherein the sequence of at least 14 contiguous nucleotides comprises a sequence of 5 contiguous nucleotides of SEQ ID NO: 484, or the complement thereof, within the sequence of nucleotides from position 132366 to132374, that comprises said nucleotide at position 132370.

235. The method of claim 232, wherein the sequence of at least 14 contiguous nucleotides is a sequence of at least 14 contiguous nucleotides of SEQ ID NO: 484, or the complement thereof, that comprises said nucleotide position at 132370.

236. The method of claim 233, wherein the coding nucleotide sequence, promoter and portion of the KNSL1 gene are contained in a nucleotide sequence that includes sequence that is heterologous to the KNSL1 gene.

237. The method of claim 232, wherein the nucleotide sequence comprising a portion of the KNSL1 gene is heterologous to the cell or animal.

238. The method of claim 232, wherein the cell is a recombinant 30 cell.

239. The method of claim 232, wherein the animal is a non-human transgenic animal.

240. The method of claim 232, wherein the promoter comprises a KNSL1 gene promoter.

241. The method of claim 232, wherein the neurodegenerative disease is Alzheimer's disease.

242. The method of claim 232, wherein the biological event is the level of an Aβ peptide in the cell, extracellular medium or animal.
